(19) United States
(12) Patent Application Publication
(10) Pub. No.: US 2016/0340740 A1
(43) Pub. Date: Nov. 24, 2016

(54) METHYLATION HAPLOTYPING FOR NON-INVASIVE DIAGNOSIS (MONOD)

(71) Applicant: THE REGENTS OF THE UNIVERSITY OF CALIFORNIA, San Diego, CA (US)

(72) Inventor: Kun Zhang, San Diego, CA (US)

(21) Appl. No.: 15/114,803

(22) PCT Filed: Jan. 29, 2015
(86) PCT No.: PCT/US2015/013562

§ 371 Date: Jul. 27, 2016

Related U.S. Application Data
(60) Provisional application No. 61/933,750, filed on Jan. 30, 2014.

(51) Int. Cl. C12Q 1/68

(52) U.S. Cl. CPC C12Q 1/6886; C12Q 1/6883; C12Q 2600/172; C12Q 2600/154

(57) ABSTRACT

Embodiments disclosed herein provides methods for detecting the presence of a target nucleic acid in a mixture of nucleic acids comprising: performing methylation haplotype analysis on a sample comprising a plurality of nucleic acids; and determining whether said sample includes a methylation haplotype indicative of the presence said target nucleic acid. Embodiments disclosed herein provide methods for detecting tumor in a subject and prenatal detection of fetal chromosomal abnormality using the methods for detecting the presence of a target nucleic acid disclosed herein. Further disclosed are probes and kits for methylation haplotype analysis.

### INCORPORATION BY REFERENCE TO ANY PRIORITY APPLICATIONS

This application claims the benefit of priority to U.S. Provisional Application Ser. No. 61/933,750, entitled “METHYLATION HAPLOTYPING FOR NON-INVASIVE DIAGNOSIS (MONOD),” filed Jan. 30, 2014, the disclosure of which is hereby incorporated by reference herein in its entirety.

### STATEMENT REGARDING FEDERALLY SPONSORED R&D

This invention was made with government support under grant number GM097253 awarded by the National Institute of Health. The government has certain rights in the invention.

### REFERENCE TO SEQUENCE LISTING, TABLE, OR COMPUTER PROGRAM LISTING

The present application is being filed along with a Sequence Listing in electronic format. The Sequence Listing is provided as a file entitled UCSD087-001WO_Sequence_Listing.TXT, created Jan. 28, 2015, which is 2,883 Kb in size. The information in the electronic format of the Sequence Listing is incorporated herein by reference in its entirety.

### BACKGROUND

1. Field

Embodiments described herein relate to compositions and methods for detecting the low-abundance nucleic acids at high sensitivity.

2. Description of the Related Art

Methods and compositions for detecting low-abundance nucleic acids with high sensitivity are disclosed herein. Such methods and compositions may be used, for example, to detect trisomy 21 or the presence of cancer.

For non-invasive trisomy 21 detection, existing methods are based on either fetal specific proteins, RNA transcripts, fetal specific methylation signatures on single CpG sites at multiple genomic positions (Poon et al. 2002; Chim et al, 2008; Lun et al, 2013), or subtle elevation of chromosome 21 DNA content in maternal blood (Chiu et al. 2008; Fan et al. 2008). The latter strategy has been commercialized by multiple companies in the US, and the tests are covered by a number of insurance plans.

For non-invasive cancer detection from peripheral blood, there are no mature commercial assays in the market. Assays at various stages of development include enrichment of rare circulating cancer stem cells followed by subsequent analyses, detecting of circulating microRNAs or DNA.

### SUMMARY

Embodiments disclosed herein provides methods for detecting the presence of a target nucleic acid in a mixture of nucleic acids comprising: performing methylation haplotype analysis on a sample comprising a plurality of nucleic acids; and determining whether said sample includes a methylation haplotype indicative of the presence said target nucleic acid. In some embodiments, said methylation haplotype analysis comprises determining the combinatorial methylation status of a plurality of methylation sites in a nucleic acid molecule. In some embodiments, said plurality of methylation sites comprises at least 2, at least 3, at least 4, at least 5, at least 10, at least 20, at least 40, at least 50, at least 100, at least 200, at least 300, at least 400, at least 500 or more than 500 methylation sites indicative of the presence of the target nucleic acid in the sample. In some embodiments, said plurality of methylation sites comprises at least 2 methylation sites. In some embodiments, said plurality of methylation sites comprises at least 3 methylation sites. In some embodiments, said plurality of methylation sites comprises at least 4 methylation sites. In some embodiments, said plurality of methylation sites comprises at least 5 methylation sites. In some embodiments, said plurality of methylation sites comprises at least 10 methylation sites. In some embodiments, said plurality of methylation sites are located within a region on a single nucleic acid molecule. In some embodiments, said region is about 5 bp to about 500 bp. In some embodiments, said region is about 10 bp to about 200 bp. In some embodiments, said region is about 20 bp to about 100 bp. In some embodiments, said plurality of methylation sites are located within a region selected from the chromosomal regions listed in Table 1, Table 2 and Table 4. In some embodiments, said target nucleic acid represents a percentage of the plurality of nucleic acids contained in the sample that is about 0.1% to about 10%. In some embodiments, said target nucleic acid represents a percentage of the plurality of nucleic acids contained in the sample that is less than about 5%. In some embodiments, said target nucleic acid represents a percentage of the plurality of nucleic acids contained in the sample that is less than about 3%. In some embodiments, said target nucleic acid represents a percentage of the plurality of nucleic acids contained in the sample that is less than about 2%. In some embodiments, said target nucleic acid represents a percentage of the plurality of nucleic acids contained in the sample that is less than about 1%. In some embodiments, the methods comprise determining whether said sample includes at least 2 methylation haplotypes indicative of the presence said target nucleic acid. In some embodiments, the methods comprise determining whether said sample includes at least 3 methylation haplotypes indicative of the presence said target nucleic acid. In some embodiments, the methods comprise determining whether said sample includes at least 5 methylation haplotypes indicative of the presence said target nucleic acid. In some embodiments, the methods comprise determining whether said sample includes at least 10 methylation haplotypes indicative of the presence said target nucleic acid. In some embodiments, the methods comprise determining whether said sample includes at least 20 methylation haplotypes indicative of the presence said target nucleic acid. In some embodiments, the methods comprise enriching the plurality of nucleic acids using selector probes. In some embodiments, the methods comprise enriching the plurality of nucleic acids using MeDiP. In some embodiments, the sample comprises genomic DNA. In some embodiments, said genomic DNA is bisulfite converted. In some embodiments, said bisulfite converted genomic DNA is amplified using microdroplet PCR. In some embodiments, determining the methylation status of a plurality of methylation sites comprises contacting said plurality of nucleic acids with a plurality of probes to form hybridization complexes. In some embodiments, the methods comprise modifying said plurality of probes of said hybridization complexes to form a plurality of modified probes. In some embodiments, modifying said plurality of probes comprises extending and/or ligating said plurality of probes. In some embodiments, each of said plurality of probes comprises a target-specific domain. In some embodiments, the methods comprise amplifying said plurality of modified probes. In some embodiments, each of said plurality of modified probes comprises a unique molecule identifier (UMI). In some embodiments, each of said plurality of modified probes comprises a primer complementary domain. In some embodiments, each of said plurality of modified probes comprises two primer complementary domains. In some embodiments, said plurality of probes are padlock probes. In some embodiments, said padlock probes comprise two target-specific domains and a linker sequence. In some embodiments, said padlock probes comprise sequences selected from the group consisting of SEQ ID NOs: 1-7,109. In some embodiments, the methods comprise sequencing said plurality of probes of said hybridization complexes. In some embodiments, the methods comprise size-selection of said plurality of nucleic acids. In some embodiments, the selected plurality of nucleic acids have a length of about 50 bp to about 200 bp. In some embodiments, the selected plurality of nucleic acids have a length of at least about 200 bp. In some embodiments, the methods comprise determining the methylation haplotype of the selected plurality of nucleic acids having different sizes. In some embodiments, the methods comprise comparing the methylation haplotype of the selected plurality of nucleic acids having different sizes. In some embodiments, the sample is a plasma sample. In some embodiments, the target nucleic acid is a nucleic acid associated with cancer. In some embodiments, the target nucleic acid is indicative of chromosomal abnormality. In some embodiments, the target nucleic acid is indicative of trisomy 21. In some embodiments, said methylation haplotype analysis comprises determining the methylation status of a plurality of methylation sites which are in linkage disequilibrium with one another.

Embodiments disclosed herein provide methods for detecting tumor in a subject, comprising: detecting the presence of one or more target nucleic acids in a sample comprising a plurality of nucleic acids from said subject using the methods disclosed herein, wherein the presence of said one or more target nucleic acids is indicative of the presence of tumor in said subject. In some embodiments, said sample is a plasma sample isolated from peripheral blood of said subject. In some embodiments, the methods comprise detecting the presence of at least 2 target nucleic acids in a sample comprising a plurality of nucleic acids. In some embodiments, the methods comprise detecting the presence of at least 5 target nucleic acids in a sample comprising a plurality of nucleic acids. In some embodiments, the methods comprise detecting the presence of at least 10 target nucleic acids in a sample comprising a plurality of nucleic acids. In some embodiments, the methods comprise detecting the presence of at least 100 target nucleic acids in a sample comprising a plurality of nucleic acids. In some embodiments, each of said one or more target nucleic acids comprises one or more methylation haplotypes that are tumor cell-indicative. In some embodiments, said one or more target nucleic acids are selected from the group consisting of the chromosomal regions listed in Table 1 and Table 4.

Embodiments disclosed herein provide methods for prenatal detection of fetal chromosomal abnormality, comprising: detecting the presence of two or more target nucleic acids in a sample comprising a plurality of nucleic acids from said subject using the methods disclosed herein; calculating the amount of said two or more target nucleic acids; and determining the presence or absence of fetal chromosomal abnormality by comparing the amount of said two or more target nucleic acids. In some embodiments, said sample is a plasma sample isolated from peripheral blood of a pregnant woman. In some embodiments, the methods comprise detecting the presence of at least 5 target nucleic acids in a sample comprising a plurality of nucleic acids. In some embodiments, the methods comprise detecting the presence of at least 10 target nucleic acids in a sample comprising a plurality of nucleic acids. In some embodiments, the methods comprise detecting the presence of at least 100 target nucleic acids in a sample comprising a plurality of nucleic acids. In some embodiments, the methods comprise detecting the presence of at least 1,000 target nucleic acids in a sample comprising a plurality of nucleic acids. In some embodiments, the methods comprise detecting the presence of at least 10,000 target nucleic acids in a sample comprising a plurality of nucleic acids. In some embodiments, each of said two or more target nucleic acids comprises one or more methylation haplotypes that are fetal cell-indicative. In some embodiments, said fetal chromosomal abnormality is trisomy 21 (Down syndrome), trisomy 18 (Edwards syndrome), trisomy 22 (cat eye syndrome), trisomy 13 (Patau syndrome), XO, XXX, XYY, XXY, or a combination thereof. In some embodiments, the methods comprise calculating the copy number of a chromosome. In some embodiments, the methods comprise comparing the copy number of a chromosome with a reference copy number. In some embodiments, said one or more target nucleic acids are selected from the group consisting of the chromosomal regions listed in Table 2.

Embodiments disclosed herein provide probes for methylation haplotype analysis, comprising: a first target-specific domain that hybridizes to a first location on a target nucleic acid; a primer-complementary domain; a linker sequence; a unique molecule identifier (UMI); and a second target-specific domain that hybridizes to a second location on said target nucleic acid, wherein said first target-specific domain and said second target-specific domain hybridize to adjacent sequences on a target nucleic acid. In some embodiments, said target nucleic acid is selected from the group consisting of the chromosomal locations listed in Tables 1-4. In some embodiments, said probe comprises a sequence selected from the group consisting of SEQ ID NOs: 1-7,109.

Embodiments disclosed herein provide kits for methylation haplotype analysis comprising a plurality of probes for methylation haplotype analysis, comprising: a first target-specific domain that hybridizes to a first location on a target nucleic acid; a primer-complementary domain; a linker sequence; a unique molecule identifier (UMI); and a second target-specific domain that hybridizes to a second location on said target nucleic acid, wherein said first target-specific domain and said second target-specific domain hybridize to adjacent sequences on a target nucleic acid. In some embodiments, said plurality of probes hybridizes to at least 5 nucleic acids. In some embodiments, said plurality of probes hybridizes to at least 10 nucleic acids. In some embodiments, said plurality of probes hybridizes to at least 50 nucleic acids. In some embodiments, said plurality of probes hybridizes to at least 100 nucleic acids. In some embodiments, said plurality of probes hybridizes to at least 500 nucleic acids.

Embodiments disclosed herein provide methods for identifying a methylation haplotype that is indicative of a tissue type, comprising: determining the methylation status of a plurality of methylation sites in a sample from said tissue type comprising a plurality of nucleic acids; comparing said methylation status of said plurality of methylation sites to the methylation status of said plurality of methylation sites on a reference sample; and identifying a methylation haplotype that is indicative of said tissue type.

Embodiments disclosed herein further provide methods of performing methylation haplotype analysis on a nucleic acid, comprising: determining the methylation status of a plurality of methylation sites on said nucleic acid, wherein said plurality of methylation sites are located on a chromosomal region.

### BRIEF DESCRIPTION OF THE DRAWINGS

FIG. 1 illustrates sensitive detection and quantification of circulating placenta or tumor DNA in blood plasma using DNA methylation haplotypes. Each line represents a single DNA molecule, and each circle is a CpG site. Open circle is unmethylated CpG and solid circle is methylated CpG.

FIG. 2 illustrates differential haplotype analysis by fragment size. Targeted capture and sequencing of long and short fragments improve sensitivity by subtraction.

FIGS. 3A and 3B illustrate multiplex capture of MONOD marker panel with umi-BSPP. FIG. 3A shows: each umi-BSPP probe contains a randomized unique molecule identifier (UMI) for uniquely labelling the capture product from each template DNA molecule. FIG. 3B shows: bisulfite converted DNA from clinical specimens is converted into sequencing libraries with 5 steps in single-tube reactions.

FIGS. 4A and 4B illustrate deriving methylation haplotypes from sequencing data. FIG. 4A shows: the bam2hapInfo program takes mapped raw sequencing data in the bam format, and reports haplotypes and their counts in hapInfo files. FIG. 4B shows: an example on deriving 4-locus methylation haplotypes from raw sequencing reads.

FIG. 5 shows quantitative detection of cancer DNA in whole blood. Top panel: level of cancer DNA detected in mixture of cancer and blood DNA at various ratios. Bottom panel: statistical significance of detection.

FIG. 6 shows detection of cancer specific aneuploidy on chromosome 21 (top panel) and chromosome 18 (bottom panel).

FIG. 7 shows tumor-specific haplotypes detected in the plasma of four cancer patients. Each row represents a methylation haplotype derived from a unique DNA molecule in the sample. Each column is the genomic position of a CpG site. The methylation status of a CpG site in each haplotype is represented as a square. Black is methylated, white is unmethylated and light blue is missing value.

### DETAILED DESCRIPTION OF THE PREFERRED EMBODIMENT

### Definitions

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

As used herein, the singular forms “a”, “an”, and “the” include plural references unless indicated otherwise, expressly or by context. For example, “a” dimer includes one or more dimers, unless indicated otherwise, expressly or by context.

The term “methylation haplotype” refers to the combination of methylation status at two or more methylation sites of a single nucleic acid molecule. As used herein, a methylation haplotype is indicative of a tissue sample, such as a tumor cell, a fetal cell, etc. In some embodiments, the methylation status at all the methylation sties of a methylation haplotype shows linkage disequilibrium. Therefore, detecting the methylation status of one methylation site may be sufficient to detect the methylation haplotype. A “methylation site,” also called a “site” or a “locus,” as used herein refers to a CpG site, but could also refer to other methylation sites, such as CHG, CHH, etc. A methylation haplotype may include the methylation status of at least 2, at least 3, at least 4, at least 5, at least 6, at least 10, at least 20, at least 40, at least 50, at least 100, at least 200, at least 300, at least 400, at least 500 or more than 500, or a range between two of any of the above values, methylation sites. The spacing between two adjacent methylation sites can be anywhere between one to hundreds of bases. The chromosomal region that covers all the methylation sites of a methylation haplotype is referred to as a “marker region,” which may vary in length. For example, a marker region may have a length that is, is about, is less than, or is more than, 4 nucleotides (nt), 5 nt, 6 nt, 7 nt, 8 nt, 9 nt, 10 nt, 20 nt, 30 nt, 40 nt, 50 nt, 60 nt, 70 nt, 80 nt, 90 nt, 100 nt, 150 nt, 200 nt, 300 nt, 400 nt, 500 nt, 1,000 nt, etc., or a range between two of any of the above values, e.g., about 5 nt to about 20 nt, about 50 nt to about 100 nt, etc.

The term “chromosomal abnormality” refers to a deviation between the structure of the subject chromosome and a normal homologous chromosome. The term “normal” refers to the predominate karyotype or banding pattern found in healthy individuals of a particular species. A chromosomal abnormality can be numerical or structural, and includes but is not limited to aneuploidy, polyploidy, inversion, a trisomy, a monosomy, duplication, deletion, deletion of a part of a chromosome, addition, addition of a part of chromosome, insertion, a fragment of a chromosome, a region of a chromosome, chromosomal rearrangement, and translocation. A chromosomal abnormality can be correlated with presence of a pathological condition or with a predisposition to develop a pathological condition.

Monosomy X (XO, absence of an entire X chromosome) is the most common type of Turner syndrome, occurring in 1 in 2500 to 1 in 3000 live-born girls (Sybert and McCauley (2004) N Engl J Med 351:1227-1238). XXY syndrome is a condition in which human males have an extra X chromosome, existing in roughly 1 out of every 1000 males (Bock, Understanding Klinefelter Syndrome: A Guide for XXY Males and Their Families. NIH Pub. No. 93-3202 (1993)). XYY syndrome is an aneuploidy of the sex chromosomes in which a human male receives an extra Y chromosome, giving a total of 47 chromosomes instead of the more usual 46, affecting 1 in 1000 male births while potentially leading to male infertility (Aksglaede et al., (2008) J Clin Endocrinol Metab 93:169-176).

Turner syndrome encompasses several conditions, of which monosomy X (XO, absence of an entire sex chromosome, the Barr body) is most common. Typical females have two X chromosomes, but in Turner syndrome, one of those sex chromosomes is missing. Occurring in 1 in 2000 to 1 in 5000 phenotypic females, the syndrome manifests itself in a number of ways. Klinefelter's syndrome is a condition in which human males have an extra X chromosome. In humans, Klinefelter's syndrome is the most common sex chromosome disorder and the second most common condition caused by the presence of extra chromosomes. The condition exists in roughly 1 out of every 1,000 males. XYY syndrome is an aneuploidy of the sex chromosomes in which a human male receives an extra Y chromosome, giving a total of 47 chromosomes instead of the more usual 46. This produces a 47, XYY karyotype. This condition is usually asymptomatic and affects 1 in 1000 male births while potentially leading to male infertility.

Trisomy 13 (Patau syndrome), trisomy 18 (Edward syndrome) and trisomy 21 (Down syndrome) are the most clinically important autosomal trisomies and how to detect them has always been the hot topic. Detection of above fetal chromosomal aberration has great significance in prenatal diagnosis (Ostler, Diseases of the eye and skin: a color atlas. Lippincott Williams & Wilkins. pp. 72. ISBN 9780781749992 (2004); Driscoll and Gross (2009) N Engl J Med 360: 2556-2562; Kagan et al., (2008) Human Reproduction 23:1968-1975).

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### Methods for Detecting a Target Nucleic Acid

In this disclosure, methods and compositions for digital quantification of methylation haplotypes, and the uses thereof to detect low abundance nucleic acids, are provided. To detect and quantify low abundance nucleic acids, such as tumor DNA or fetal DNA, marker regions in the genome in which there are major differences in methylation status between normal cells, such as normal cells in peripheral blood or other samples, and other cells of interest (such as cancer or fetal cells) are analyzed. These are the marker regions used for methylation status analysis. For example, a marker region containing 6 CpG sites might be completely unmethylated in whole blood, and fully methylated in placenta. If a maternal whole blood sample contains 3% of fetal DNA, then a 3% methylation level can be detected with an ideal assay. However, all methylation assays have technical errors, such as incomplete bisulfate conversion, incomplete enzyme digestion, and/or sequencing errors. Typically all the technical errors combined can contribute to ˜1-2%. With the presence of these errors, a 3% methylation level cannot be confidently detected using an individual methylation site. Such technical errors greatly compromise the sensitivity and confidence in detecting and quantifying fetal DNA molecules. Methylation haplotyping analysis can dramatically improve the discriminating power by minimizing the effect of the technical errors. In this case, for example, the 3% fetal DNA molecules are fully (or almost fully) methylated at all CpG sites in a marker region, whereas the maternal DNA molecules are not methylated in this marker region. In other words, the methylation status at multiple CpG sites in a marker region on the same nucleic acid are all linked. Using a methylation haplotype comprising four or more CpG sites, one can confidently identify rare DNA molecules that are present at a percentage of 0.01% or even lower, at least two orders of magnitude below the technical errors (1-2% per site) for a single site. An analytical framework for methylation haplotype and linkage disequilibrium analysis has been developed (Shoemaker et al, 2009). In addition, combining information from multiple marker regions will further improve the sensitivity and robustness in the presence of biological variability. Without being bound by a theory, cell-free DNA typically comes from apoptotic cells, and is in short fragments (Chan et al. 2004). In contrast, whole blood DNA typically has larger sizes (at least kilobases) even after DNA extraction. Analysis of methylation haplotypes from DNA molecules of different sizes adds another level of stringency in detecting rare target nucleic acid molecules.

In some embodiments, methylation haplotypes (as opposed to individual CpG sites) are used to improve detection sensitivity and reduce technical noise for detecting low abundance species in mixed DNA samples. A methylation haplotype is the combinatorial (or linked) methylation status of multiple methylation sites (also called “sites” or “loci”) in a single DNA molecule. Multi-locus haplotypes are robust with the presence of random technical errors on individual loci, and hence provide a much greater power in distinguishing true signatures at a low abundance from technical errors. As used herein, a methylation haplotype is indicative of a tissue sample, such as a tumor cell, a fetal cell, etc.

The methods and compositions described herein allow the deconvolution of mixed nucleic acid samples, such as DNA samples, based on the detection of low-abundance nucleic acids at extremely high sensitivity. In some embodiments, the low-abundance nucleic acids may be from a particular tissue or cell type, such as a fetal cell or cancer cell. In some embodiments, methylation haplotypes in a set of marker regions in the genome are used. For example, the methylation haplotype may include at least 5, at least 10, at least 20, at least 40, at least 50, at least 100, at least 200, at least 300, at least 400, at least 500 or more than 500 methylation sites indicative of the presence of the target nucleic acid in the sample. In some embodiments, the methods and compositions disclosed herein may be used to separate and detect rare nucleic acids, for example low abundance DNA in a mixture of DNA, based on methylation haplotypes or epigenetic modifications (cytosine methylation) on multiple CpG sites of single DNA molecules. In some embodiments, high detection sensitivity is provided, which translates to detection at earlier time point. In some embodiments, standard short-read sequencing platforms may be used. In some embodiments, the disclosed methods and compositions provide lower assay cost compared to the other existing methods.

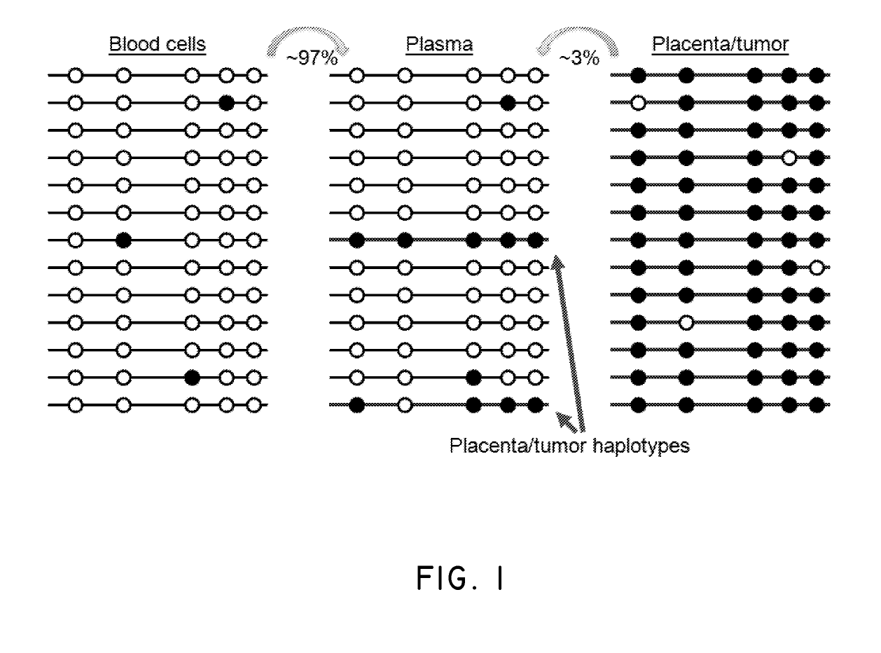

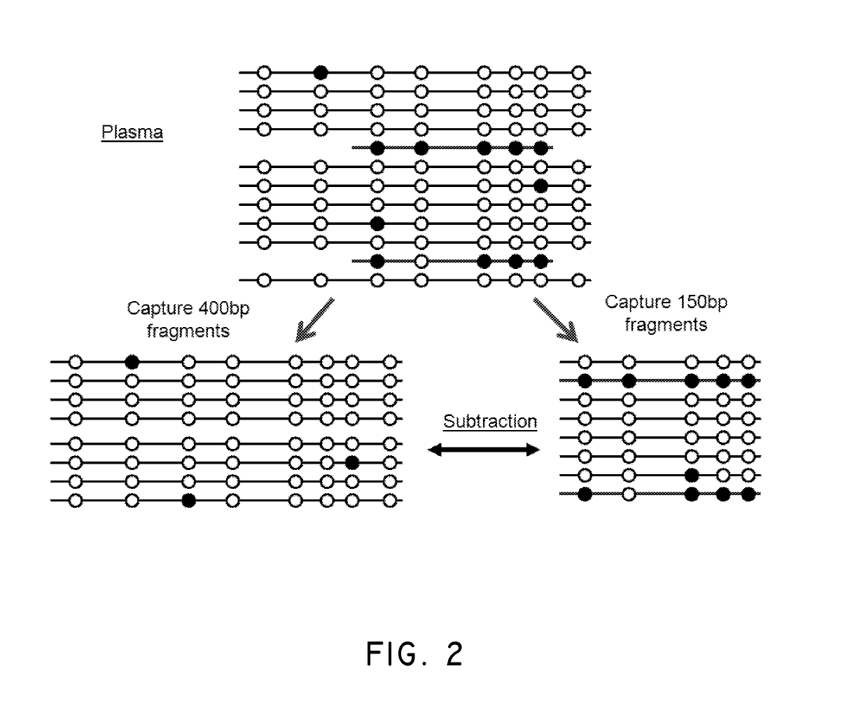

In some embodiments, the methods and compositions disclosed herein provide ultra-high sensitivity for detecting a rare target nucleic acid species from a sample comprising a plurality of nucleic acids (such as peripheral blood DNA) using some combinations of the three concepts: (i) multi-locus methylation haplotype analysis (FIG. 1); (ii) integrative analysis of multiple marker regions; and (iii) differential haplotype analysis of DNA fragments with different sizes (FIG. 2). Embodiments disclosed herein are capable of detecting the presence of a target nucleic acid that represents a small fraction of a plurality of nucleic acids contained in a sample. For example, in some embodiments, the target nucleic acid may represent a percentage of the plurality of nucleic acids that is, is about, is less than, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a range between two of any of the above values, e.g., about 0.1% to about 2%, about 1% to about 3%, etc. In some embodiments, the target nucleic acid may represent a percentage of the plurality of nucleic acids that is less than about 5%. In some embodiments, the target nucleic acid may represent a percentage of the plurality of nucleic acids that is less than about 3%. In some embodiments, the target nucleic acid may represent a percentage of the plurality of nucleic acids that is less than about 2%. In some embodiments, the target nucleic acid may represent a percentage of the plurality of nucleic acids that is less than about 1%. In some embodiments, the methods and compositions disclosed herein can be implemented as genetic screening or diagnostic tests for non-invasive prenatal diagnosis, non-invasive monitoring of tumor loads in cancer patients after treatments, or early-stage cancer detection.

In some embodiments, a nucleic acid sample includes a double-stranded nucleic acid. In some embodiments, a nucleic acid sample includes genomic DNA, or cDNA. In some embodiments, mitochondrial or chloroplast DNA is used. In some embodiments, a nucleic acid sample includes RNA or derivatives thereof such as mRNA or cDNA. Some embodiments described herein can utilize a plurality of different nucleic acid species (e.g., nucleic acid molecules having different nucleotide sequences being present in the plurality).

In some embodiments, two or more methylation haplotypes may be determined that are indicative of the presence of a target nucleic acid, or a plurality of target nucleic acids. For example, at least 2, at least 3, at least 5, at least 10, at least 20, or a range between two of any of the above values, methylation haplotypes may be determined that are indicative of the presence of a target nucleic acid. The chromosomal location of the two or more methylation haplotypes may vary. For example, the two or more methylation haplotypes may be located on the same or different chromosomes. In some embodiments, wherein the plurality of target nucleic acids are located on the same chromosome, two or more methylation haplotypes located on the same chromosome may be determined. In some embodiments, the plurality of nucleic acids from the sample may be subject to size-selection before methylation status analysis. For example, in some embodiments, the plurality of nucleic acids may have a size that is, is about, is at least, is at most, 20 bp, 30 bp, 40 bp, 50 bp, 60 bp, 70 bp, 80 bp, 90 bp, 100 bp, 150 bp, 200 bp, 300 bp, 400 bp, 500 bp, or a range between two of any of the above values, e.g., about 50 bp to about 200 bp, about 100 bp to about 250 bp, etc. In some embodiments, the methylation haplotype of the selected plurality of nucleic acids having different sizes are determined. In some embodiments, the methylation haplotype of the selected plurality of nucleic acids having different sizes are compared. Integrating methylation haplotypes from multiple chromosomal locations, and differential methylation haplotype analysis of DNA fragments with different sizes further increases the sensitivity of the methods of detection. A higher sensitivity translates to robust detection at earlier time points and/or lower assay costs.

### Analysis of Methylation Status

In some embodiments of the methods and compositions described herein, methylation haplotypes, or epigenetic modifications (cytosine methylation) on multiple methylation sites of single nucleic acid molecules are analyzed by determining the methylation status of the methylation sites covered by the methylation haplotype. As used herein, “methylation status” refers to methylation or unmethylation of a cytosine residue, for example, in a CpG dinucleotide, or in other contexts, e.g., CHG, CHH, etc. Methylated cytosine may be a variety of forms, for example, 5-methylcytosine (5mC), 5-hydroxymethylcytosine (5hmC), 5-formylcytosine (5fC) and 5-carboxycytosine (5caC), etc. A variety of protocols are available for the analysis of methylation status, with or without target enrichment (reviewed in Plongthongkum et al. 2014). For example, reduced representation bisulphite sequencing (RRBS), methylation restriction enzyme sequencing (MRE-seq), methylation DNA immunoprecipitation sequencing (MeDIP-seq, Papageorgiou et al. 2010), methyl-CpG-binding domain protein sequencing (MBD-seq), methylation DNA capture sequencing (MethylCap-seq), etc. In some embodiments, methylation status can be obtained by the standard short-read sequencing platforms, such as Illumina's HiSeq/MiSeq or LifeTech's Ion Proton, as part of the methylation assay without extra efforts and cost. In some embodiments, the methylation status may be analyzed using a target methylation sequencing technology (e.g., Bisulfite Padlock Probes, or BSPP, Deng et al, 2008; Diep et al. 2012).

In some embodiments, the target nucleic acids may be enriched before the methylation status analysis. For example, micro-droplet PCR (Komori et al. 2011) or Selector probes (Johansson et al. 2011) can be used with some differences in the requirement of input materials and/or cost. Accordingly, each of the foregoing approaches may be utilized in some embodiments of the methods and compositions described herein.

In preferred embodiments, the methylation status analysis methods disclosed herein take the bisulfite sequencing reads (single-ends or paired-ends) as the input.

Further provided are methods of performing methylation haplotype analysis on a nucleic acid, comprising: determining the methylation status of a plurality of methylation sites on the nucleic acid, wherein said plurality of methylation sites are located on a marker region. Using the results from methylation status analysis, methylation haplotypes and their abundance from the raw sequencing reads are derived. For sample preparation methods that allow identifying multiple clonal sequencing reads originated from the same template DNA molecules (such as umi-RRBS, or hybridization-based target capture), the consensus methylation haplotypes may be derived from the clonal reads to improve the accuracy and avoid over-dispersion of methylation haplotype counts. The probability that a methylation haplotype is present in a sample (or a pool of samples) is determined by the frequency the exact methylation haplotype is observed, or estimated from the methylation levels of individual CpG sites and the technical error rates (sequencing errors, bisulfite conversion errors) assuming no linkage between adjacent sites (or P(M1M2)=P(M1)*P(M2) where M1M2 is the probability of two-locus methylated haplotype, P(M1) and P(M2) are the mean methylation level at the two loci in the cell population or tissue).

Therefore, further provided are methods for identifying a methylation haplotype that is indicative of a tissue type, comprising: determining the methylation status of a plurality of methylation sites on a sample from said tissue type comprising a plurality of nucleic acids; comparing said methylation status of said plurality of methylation sites to the methylation status of said plurality of methylation sites on a reference sample; and identifying a methylation haplotype that is indicative of said tissue type. Preferably, the plurality of methylation sites are located in the same chromosomal location, or a marker region. Methylation haplotypes that are indicative of a tissue type, such as tumor cell or fetal cell, may be identified where a plurality of methylation sites show linkage disequilibrium between the tissue type and a reference tissue type, such as normal blood cells. For example, for each methylation haplotype from a plasma sample of a subject having tumor, the likelihoods of it originating from the pooled tumor primary biopsies data and from the pooled normal plasma data are determined, and calculated the negative log likelihood ratio. In some embodiments, a methylation haplotype is classified as indicative of a tissue type when the negative log likelihood ratio is, is about, is at least 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, or a range between two of any of the above values, e.g., about 2.0 to about 4.0, about 3.0 to about 5.0, etc. A methylation haplotype may include the methylation status of at least 2, at least 3, at least 4, at least 5, at least 6, at least 10, at least 20, at least 40, at least 50, at least 100, at least 200, at least 300, at least 400, at least 500 or more than 500, or a range between two of any of the above values, methylation sites. In some embodiments, methylation haplotypes that contain four or more methylation sites are preferred.

### Methods of Clinical Diagnosis

The disclosed methods and compositions can be used in a number of contexts, such as clinical diagnostic tests. For example, the disclosed methods and compositions may be used in non-invasive methods for detection of fetal chromosomal abnormality, e.g., trisomy 21, by analyzing fetal DNA in maternal whole blood at a very early stage of pregnancy, or non-invasive detection of circulating tumor cell-indicative DNA in patient's whole blood. Human peripheral blood contains low levels of DNA molecules from other tissues or cell types, such as circulating cancer stem cells or cell-free DNA from apoptotic cancer cells in cancer patients, or fetal DNA in pregnant women.

Therefore, some embodiments disclosed herein provide methods for detecting tumor in a subject, comprising: detecting the presence of one or more target nucleic acids in a sample comprising a plurality of nucleic acids from said subject using the methods and compositions disclosed herein, wherein the presence of said one or more target nucleic acids is indicative of the presence of tumor in said subject. The number of target nucleic acids detected may vary according to the specific needs of a particular tumor and/or subject. For example, in some embodiments, the number of target nucleic acids detected is, is about, is less than, is more than, 2, 3, 4, 5, 6, 7, 8, 9, 10, 20, 50, 100, 1,000, 10,000, or a range between any two of the above values, e.g., about 2 to about 10, about 5 to about 100, etc. In some embodiments, the number of target nucleic acids detected is at least 2. In some embodiments, the number of target nucleic acids detected is at least 5. In some embodiments, the number of target nucleic acids detected is at least 10. In some embodiments, the number of target nucleic acids detected is at least 100. In preferred embodiments, each of the target nucleic acids comprises one or more methylation haplotypes that are tumor cell-indicative.

In some embodiments, the methods and compositions disclosed herein provide high-sensitivity detection and are simple to utilize.

Haplotypes have been used for fetal genome sequencing from maternal blood (Kitzman et al. 2012; Fan et al., 2012). However, these methods focus on genetic variants and long-range haplotypes of genetic variants. Such haplotypes have to be determined by additional methods, such as clone-based haplotyping, or single chromosome isolation and sequencing.

Therefore, some embodiments disclosed herein provide methods for prenatal detection of fetal chromosomal abnormality, comprising: detecting the presence of one or more target nucleic acids in a sample comprising a plurality of nucleic acids from said subject using the methods and compositions disclosed herein; and determining the presence or absence of fetal chromosomal abnormality by comparing the level of said one or more target nucleic acids to a reference level. The number of target nucleic acids detected may vary according to the specific needs of a particular tumor and/or subject. For example, in some embodiments, the number of target nucleic acids detected is, is about, is less than, is more than, 2, 3, 4, 5, 6, 7, 8, 9, 10, 20, 50, 100, 1,000, 10,000, or a range between any two of the above values, e.g., about 10 to about 10, about 100 to about 1,000, etc. In some embodiments, the number of target nucleic acids detected is at least 2. In some embodiments, the number of target nucleic acids detected is at least 5. In some embodiments, the number of target nucleic acids detected is at least 10. In some embodiments, the number of target nucleic acids detected is at least 100. In some embodiments, the number of target nucleic acids detected is at least 1,000. In some embodiments, the number of target nucleic acids detected is at least 10,000. In preferred embodiments, each of the target nucleic acids comprises one or more methylation haplotypes that are fetal cell-indicative.

In some embodiments, the methods and compositions described herein allow separation of fetal DNA from maternal DNA in a sample, such as peripheral blood sample, from a pregnant woman, based on methylation haplotypes for copy number estimation. This may lead to a higher detection sensitivity and a lower assay cost.

In one embodiment, the sample contains both maternal DNA and fetal DNA. In another embodiment, the sample is obtained from peripheral blood of a pregnant female. Blood may be collected using any standard technique for blood drawing including but not limited to venipuncture. For example, blood can be drawn from a vein from the inside of the elbow or the back of the hand. Blood samples can be collected from a pregnant female at any time during fetal gestation. For example, blood samples can be collected from human females at about or before 4, 5, 6, 7, 8, 9, 10, 11, 12, 16, 20, 24, 28, 32, 36, 40, weeks of fetal gestation, or a time between two of any of the above time points, and preferably between 8-28 weeks of fetal gestation.

### Probes for Methylation Haplotype Analysis

Embodiments disclosed herein further provide probes for methylation haplotype analysis, comprising: a first target-specific domain that hybridizes to a first location on a target nucleic acid; a primer-commentary domain; a linker sequence; a unique molecule identifier (UMI); and a second target-specific domain that hybridizes to a second location on said target nucleic acid, wherein said first target-specific domain and said second target-specific domain hybridize to adjacent sequences on a target nucleic acid. In some embodiments, the target nucleic acid is selected from the group consisting of the chromosomal locations listed in Table 1 and Table 2. In some embodiments, the probe comprises a sequence selected from the group consisting of SEQ ID NOs: 1-7,109.

In some embodiments, provided herein are methods for constructing a sequencing library using a plurality of the probes disclosed herein. Nucleic acid molecules, preferably genomic DNA, may be purified from a sample using a protocol that preserves the methylation status of the nucleic acids. Subsequently, the purified nucleic acid, e.g., genomic DNA, is bisulfite converted. In some embodiments, sodium bisulfite treatment may be used to convert unmethylated cytosine into uracil, which is replaced by thymine after amplification (e.g., PCR), while 5-methylcytosine remains unchanged. As outlined herein, there are a variety of bisulfite conversion kits available. For example, use of the Zymo EZ DNA Methylation-Lightning Kit (Zymo Research, Irvine, Calif.) bisulfite conversion protocol is sufficient for conversion of >98% of the non-methylated cytosines. In some embodiments, the purified genomic DNA may be subject to fragmentation, for example, by sonication, endonuclease digestion, etc.

The library construction protocol may also include a fragment size selection step. For example, the fragment size selection step may be a bead-based protocol that is used to select fragments within a desired base pair range. In some embodiments, SPRIselect beads (Beckman Coulter) may be used to select fragments in a certain base pair range. In some embodiments, Agencourt AmPure® XP system (Beckman Coulter) may be used to select fragments in a certain base pair range.

In some embodiments, the methods comprise contacting a plurality of nucleic acids with a plurality of probes to form hybridization complexes. The first target specific domain and the second target-specific domain of the probe may hybridize to a marker region that covers a methylation haplotype. In preferred embodiments, the target-specific domains hybridize to sequences of the marker region that are not modified by methylation, but are adjacent to all or some of the methylation sties of the methylation haplotype.

The hybridized probes may be modified, for example, by extension and/or ligation, to form a circularized probe. After digestion of the single-stranded nucleic acids, the circularized probes may be amplified with a primer that anneals to the primer-complementary domain. In some embodiments, the amplification enzyme, e.g., DNA polymerase, may be capable of strand displacement. In some embodiments, a DNA polymerase without strand displacement activity may be used to generate a linearized nucleic acid molecule. The linearized nucleic acid molecule may be further amplified by PCR. Accordingly, in some embodiments, the probe may comprise a second primer-complementary domain. The primer may include a variety of tags and/or adaptors, such as sequencing adaptor, sample tag or barcode, etc.

### Kits

Embodiments disclosed herein further provide kits for methylation haplotype analysis comprising a plurality of probes for methylation haplotype analysis, comprising: a first target-specific domain that hybridizes to a first location on a target nucleic acid; a primer-commentary domain; a linker sequence; a unique molecule identifier (UMI); and a second target-specific domain that hybridizes to a second location on said target nucleic acid, wherein said first target-specific domain and said second target-specific domain hybridize to adjacent sequences on a target nucleic acid.

### EXAMPLES

The following examples are offered to illustrate but not to limit the invention.

In order to facilitate understanding, the specific embodiments are provided to help interpret the technical proposal, that is, these embodiments are only for illustrative purposes, but not in any way to limit the scope of the invention. Unless otherwise specified, embodiments do not indicate the specific conditions, are in accordance with the conventional conditions or the manufacturer's recommended conditions.

### Example 1

### Bisulfite Conversion

For tumor genomic DNA, bisulfite conversion was carried out on 500 ng input genomic DNA using EZ-96 DNA Methylation Lightening MagPrep (Zymo Research) following the protocol from manufacturer. Bisulfite converted DNA was measured with Qubit ssDNA assay, and recovery rate was calculated.

For plasma DNA(10-50 ng), WGBS libraries were made by first performing end-repair and T-tailing using KAPA Hyper Prep kit, and the resulted DNA fragments were ligated with Illumina methylated TruSeq adaptors, followed with bisulfite conversion using the Zymo EZ DNA Methylation Lightning kit. The bisulfite converted DNA were PCR amplified with 11-12 cycles using the PfuTurbo Cx polymerase, and purified with AMPure beads (Beckman Coulter).

### Example 2

### BSPP Capture

Approximately 150 ng of bisulfite treated DNA or WGBS library was mixed with pooled probes at 1:200 molar ratio in 1× AmpLigase buffer (Epicentre) in total volume 20 ul in 0.2 mL 8-tube strip. The reaction mixture was overlaid with 40 ul mineral oil to prevent evaporation during padlock probe capture. The reaction mixture was incubated on thermocycler with the following thermocyling program; 95 C for 30 s, cool down to 54 C at 0.02 C/s, hold at 55 C for 20 hr. 2 ul of SLN solution mix was added to the capture reaction (KLN: 2 U/ul NEB Hemo KlenTaq, 0.5 U/ul AmpLigase (Epicentre); 100 uM dNTP, 1× AmpLigase buffer) and continued to incubate at 55 C for 20 hr to fill the gap between the capturing arms and to ligate resulting in circularized DNA. The enzyme was inactivated in the previous step by heating at 94 C for 2 min. Bisulfite converted DNA and free BSPP in the mixture was removed by adding 1 ul each of Exonuclease I (Epicentre) and Exonuclease III (Epicentre) and incubated at 37 C for 2 hr. Exonuclease enzymes was heat inactivated at 94 C for 5 min and stored captured DNA at 4 C or performed amplification immediately.

### Example 3

### Amplification and Sequencing Library Construction

Captured DNA was amplified in small volume to monitor the number of cycle to amplify and verify if the capture work. 2.5 ul of circularized DNA was added to 1× KAPA SYBR Fast qPCR Master Mix with 200 nM each of AmpF6.4.Sol and AmpR6.3.Index primers in total volume 25 ul and incubated the reaction at 98° C. for 30 s, 8 cycles of 98° C. for 10 s, 58° C. for 20, 72° C. for 20 s, 15 cycles of 98° C. for 10 s, 72° C. for 20, and 72° C. for 3 min. PCR product was purified with 0.8 volume of AMPure beads (Beckman Coulter) following the protocol provided by manufacturer and eluted with 30 ul TE buffer. 3 ul of amplified amplicons was verified in 6% TBE gel. The optimal cycle number and the right expected amplicon size were obtained, and used to perform PCR in larger volume. The rest of captured DNA was amplified by adding 10 ul of circularized DNA in total 50 ul reaction with 200 nM each of AmpF6.4.Sol and AmpR6.3.Index primer, 1×KAPA SYBR Fast qPCR Master Mix in duplicates, and incubated on thermocycler as the thermocycling program above. 100 ul of PCR product was pooled, purified with 0.8 volume of AMPure bead, eluted with 60 ul EB buffer, and verified 3 ul in 6% TBE gel. The concentration of each library was determined by PAGE quantification. Each library in the same pool was combined with equimolar ratio and PAGE-size selected by cutting the smear between 475 bp-500 bp. Sequencing libraries were resuspended with approximately 20 ul H₂O and performed qPCR to quantified concentration of the pooled libraries. The libraries were run in Illumina MiSeq run (PE, 250 bp+6 bp+250 bp) with SolSeq6.3.3 (Read1), SolSeqV6.3.2r (Read2), and AmpR6.3IndSeq(IndexRead) primers.

| Primer name | Primer sequences |
|---|---|
| pAP1V61U | 5′-G*G*G*TCATATCGGTCACTGTU-3′ |
| AP2V6 | 5′-/5Phos/CACGGGTAGTGTGTATCCTG-3′ |
| pAP1V41U | 5′-G*T*AGACTGGAAGAGCACTGTU-3′ |
| AP2V4 | 5′-/Phos/TAGCCTCATGCGTATCCGAT-3′ |
| RE-DpnII-V6 | 5′-GTGTATCCTGATC-3′ |
| RE-DpnII-V4 | 5′-ATGCGTATCCGATC-3′ |
| AmpF6.4Sol | 5′-AATGATACGGCGACCACCGAGATCTACACCACTCTCAGATGTTA |
| | TCGAGGTCCGAC-3′ |
| AmpR6.3.Index | 5′-CAAGCAGAAGACGGCATACGAGATXXXXXXGCTAGGAACGATGA |
| | GCCTCCAAC-3′ |
| SolSeq6.3.3 (Read1) | 5′-TACACCACTCTCAGATGTTATCGAGGTCCGAC-3′ |
| SolSeqV6.3.2r(Read2) | 5′-GCTAGGAACGATGAGCCTCCAAC-3′ |
| AmpR6.3IndSeq(IndexRead) | 5′-GTTGGAGGCTCATCGTTCCTAGC-3′ |

### Example 4

### Identification of Candidate Marker Regions

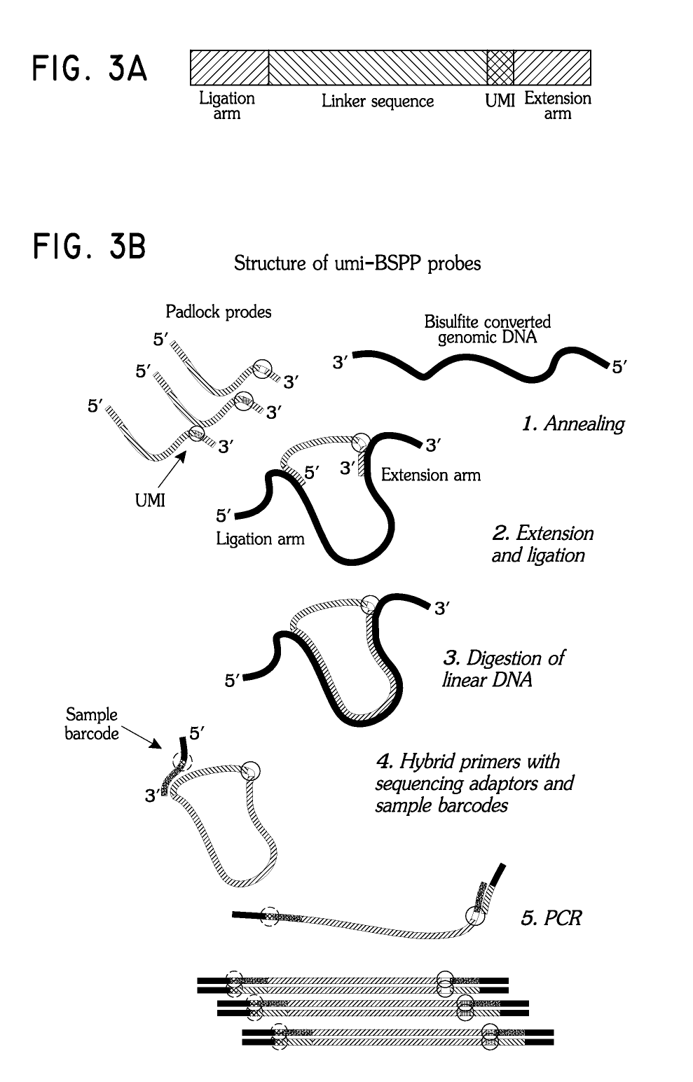

As described herein, marker regions which may be utilized in the present methods and compositions have been identified by extensive analyses of published and unpublished DNA methylation data on whole blood, cancers, and placenta. 1,484 candidate marker regions (listed in Table 1 provided below; the information in the rows and columns of this table has been provided as continuous text from which the rows and columns can be generated; in particular, rows of the table are separated by “;” and columns of each row are separated by “/”; Column 1 is Cluster ID, Column 2 is Chromosome #, Column 3 is Start Position, and Column 4 is End Position) that exhibit large methylation difference between whole blood and two types of cancer cell lines (pancreatic cancer, glioblastoma multiform, lung cancer) were identified, as well as 1,600 candidate marker regions that exhibit large methylation difference between whole blood and placenta (listed in Table 2 provided below; the information in the rows and columns of this table has been provided as continuous text from which the rows and columns can be generated; in particular, rows of the table are separated by “;” and columns of each row are separated by “/”; Column 1 is Chromosome #, Column 2 is Start Position, Column 3 is End Position, and Column 4 is Cluster ID). Umi-BSPP (FIG. 3), which is an improved version of Bisulfite Padlock Probes (BSPP, Deng et al, 2009; Diep et al. 2012) was designed for targeted methylation sequencing of these candidate marker regions of whole blood and a panel of five cancer (pancreatic cancer and glioblastoma) cell lines. These probes have built-in Unique Molecule Identifier (Kivioja et al. 2011), so that deep sequencing and true single-molecule counting can be performed to avoid quantification artefacts due to DNA amplification.

### Example 5

### Deriving Methylation Haplotypes from Sequencing Data

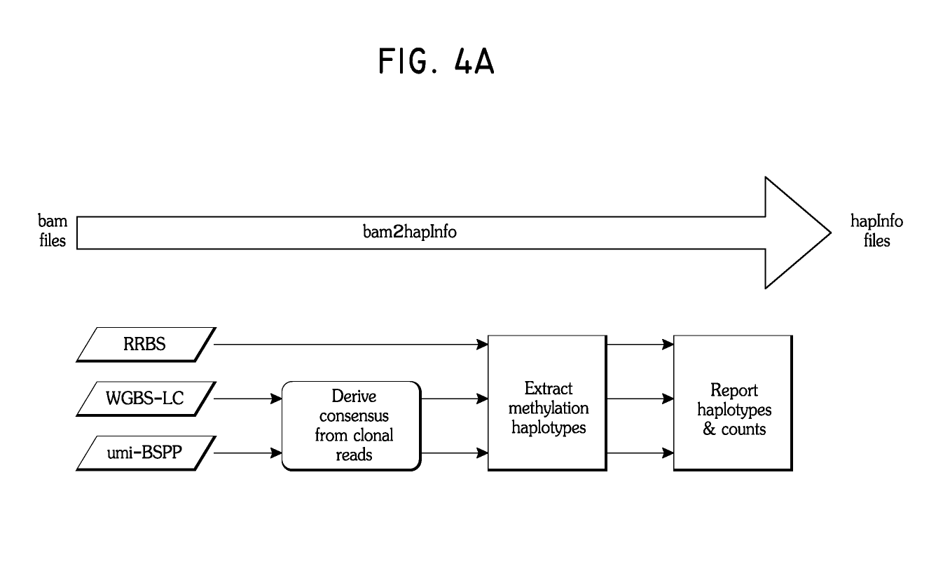

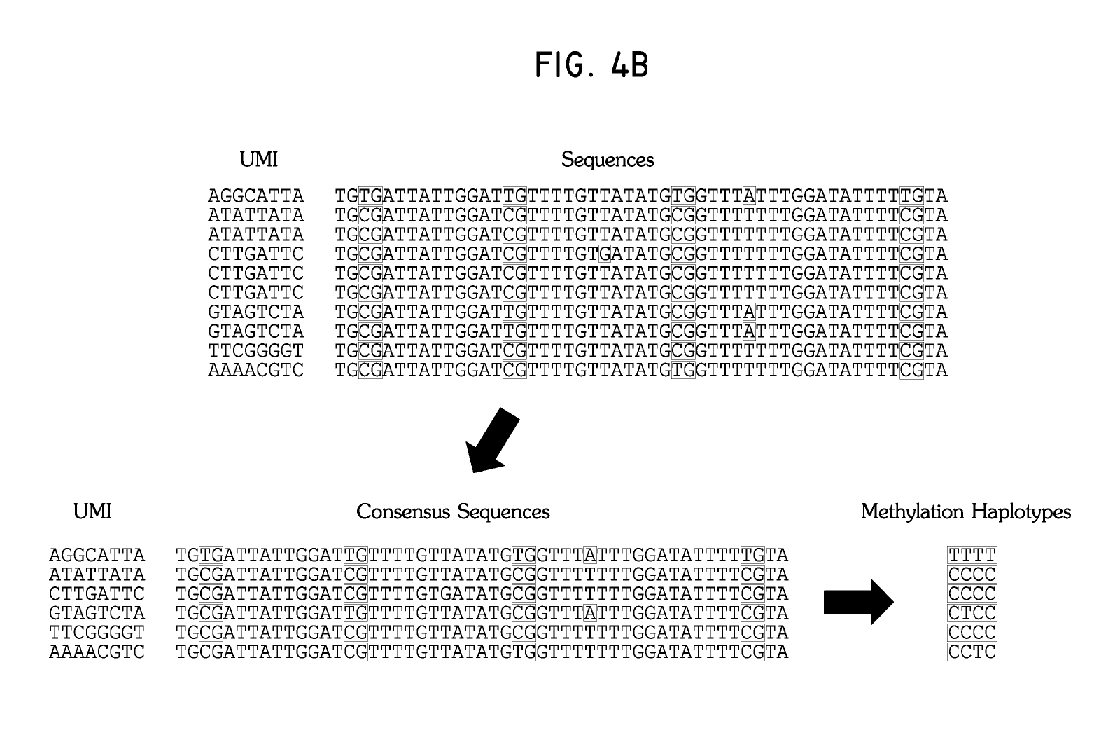

A total of 7,109 long oligonucleotides were designed and synthesized for the candidate targets, and were converted into functional bisulfite padlock probes (see Table 3 provided below and the Sequence Listing submitted herewith; the information in the rows and columns of Table 3 has been provided as continuous text from which the rows and columns can be generated; in particular, rows of Table 3 are separated by “;” and columns of each row are separated by “/”; Column 1 is SEQ ID NO, and Column 2 is Binding Site). These probes were used for targeted methylation sequencing of whole blood samples and cancer cell lines. Computational analysis of methylation haplotypes started with bisulfite sequencing reads mapped to the reference genome (using common bisulfite read mapping algorithms, such as bisReadMapper, bisMark) in the format of barn files. Consensus sequencing reads based on UMI (if available) were derived, then the methylation haplotypes on multiple CpG sites in single consensus sequencing reads (FIG. 4) were determined. The haplotypes and their counts in all genomic regions assayed were reported. For selection of the marker region set that can classify plasma samples, a methylated haplotype load (MHL) was defined for each candidate marker region, which is the normalized fraction of methylated haplotypes at different length:

$$MHL = \frac{{\sum }_{i = 1}^{i} {w}_{i} P ( {MH}_{i} )}{{\sum }_{i = 1}^{i} {w}_{i}}$$

where i is the length of haplotypes, P(MHᵢ) is the fraction of fully methylated haplotype with i loci. For a haplotype (a string) of length L, all the sub-strings with length from 1 to L were considered in this calculation. wᵢ is the weight for i-locus haplotype. wᵢ=i or wᵢ=i² were typically used to favor the contribution of longer sub-strings. After calculating MHL for all candidate regions for all samples, a MHL matrix was built for feature selection, using standard machine learning approaches such as SVM, random forest or MRMR.

### Example 6

### Detection Sensitivity and Confidence for Cancer-Specific DNA

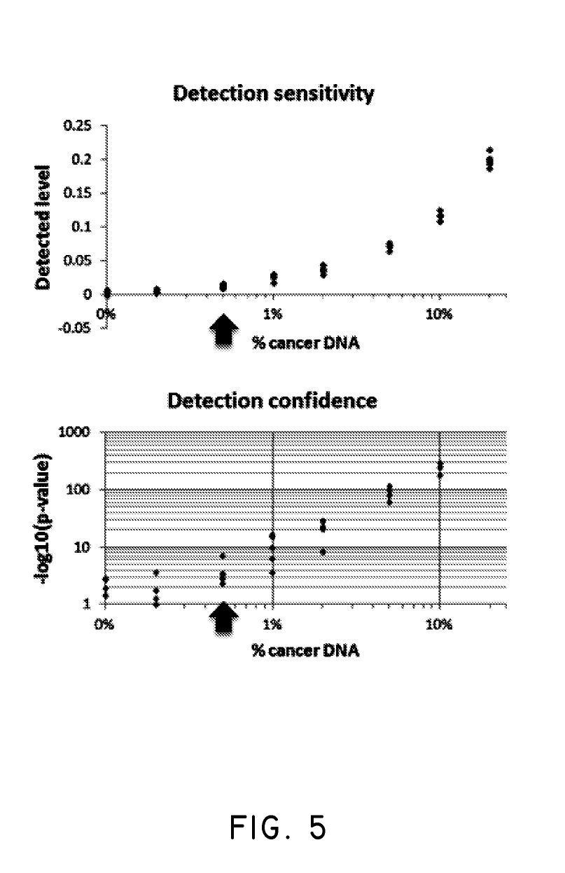

Multiple rounds of screening were performed for a subset of probes that are sensitive, robust and informative. A set of 2,489 cancer probes were selected for detecting various levels of cancer and whole blood DNA mixtures (0%, 0.2%, 0.5%, 1%, 2%, 5%, 10%, 20%) (FIG. 5). This first proof-of-concept experiment clearly indicated that multi-CpG methylation haplotypes are very effective in separating cancer DNA from whole blood for sensitive and quantitative detection.

### Example 7

### Quantifying Fetal Chromosome Dosage Using Methylation Haplotyping

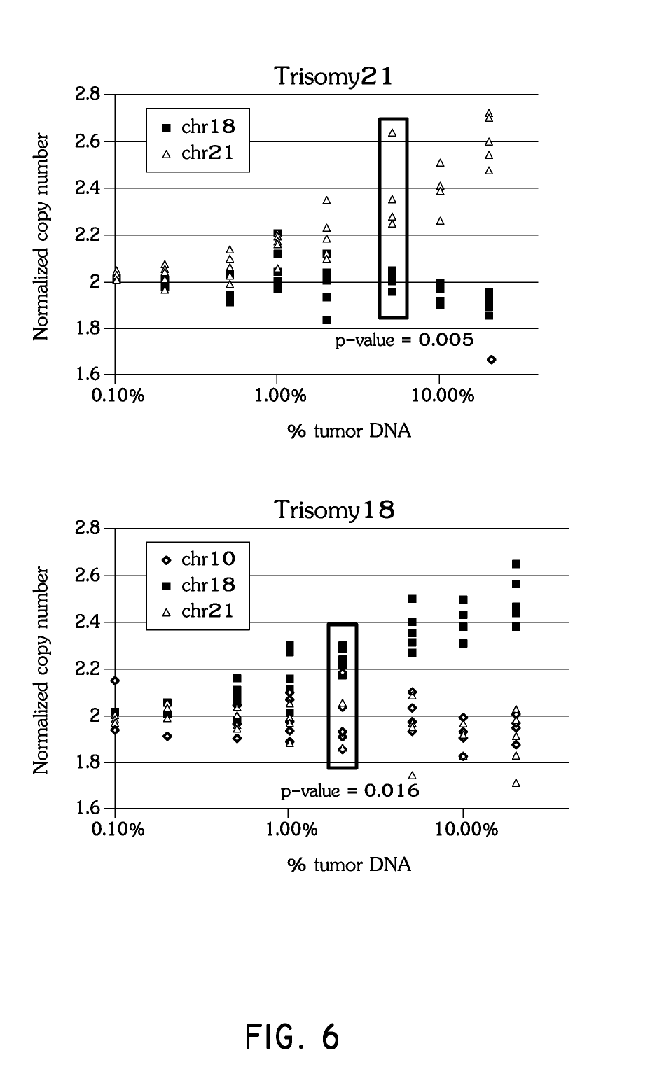

Next, the possibilities of identifying fetal indicative haplotypes and using the sequencing reads mapped to these haplotypes for quantifying fetal chromosome dosage for non-invasive prenatal diagnosis of chromosomal abnormality such as trisomy 21 in blood were tested. By subsampling the captured sequencing reads that mapped to individual chromosomes and generating synthetic mixture of cancer trisomy 18 and 21 with diploid whole blood DNA, significant gains of a single copy of chromosome 18 at 2% or chromosome 21 at 5% levels (FIG. 6) were detected. These results demonstrate that quantitative detection of chromosomal abnormality in DNA/cells that carry methylation signatures distinct from whole blood is possible with methylation haplotypes. The detection sensitivity can be further improved by additional optimization of the marker region selection and refining the statistical model when larger data sets are available.

### Example 8

### Detecting Tumor in Clinical Samples Using Methylation Haplotyping

The presently disclosed methods and compositions encompass modifications which provide further increases in sensitivity, reliability and efficiency. The power of the methylation haplotyping method increases greatly as more informative regions are included. The ˜500 informative targets in a first study came from a screen of ˜2% of the genome of which methylation data are public available. Only 42 genomic loci were used to quantify chromosome dosage. By performing whole genome bisulfite sequencing of a larger number of cancer samples and placenta, which provided methylation information in >90% of the genome, the targets were increased by 40-50 fold. It has been previously demonstrated that methylation capture and sequencing with at least 330,000 probes can be performed in one reaction at the cost of ˜$200. With additional rounds of optimization, highly informative and specific probe sets at the size of ˜10,000 and the assay cost below $50 can be developed. This should improve the limit of detection well below 0.1% for both cancer DNA detection and fetal trisomy detection.

The feasibility of detecting tumor signatures in clinical samples based on the methylation haplotypes was tested. 30 patient plasma samples were obtained, 10 from each of the three cancer types: colon cancer, lung cancer and pancreatic cancer. 15 fresh frozen primary cancer tissues were collected, 5 per cancer type from the same patients that plasma samples were collected. In addition, 30 plasma samples from normal individuals with no detectible cancer were included as the negative controls.

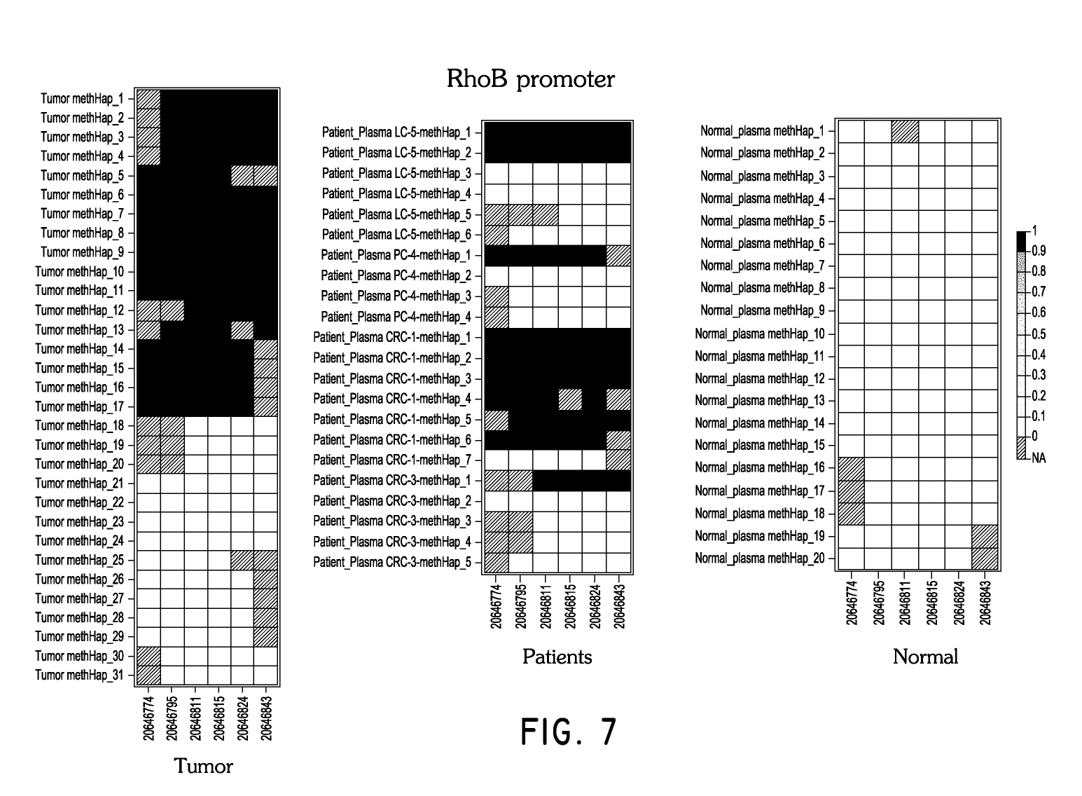

An unbiased approach to systematically screen for candidate markers appropriate for MONOD was taken. In the first strategy, the published whole genome bisulfate sequencing data from human whole blood was analyzed, and blood unmethylation regions (UMRs) were searched for. These UMRs represent the “windows” in the human genome in which tumor methylated haplotypes are not masked by blood methylation signatures if they are present in peripheral blood. A customized NimbleGen probe set was designed to capture a total of 29 Mb blood UMRs for the entire panel of 75 samples for bisulfite sequencing. In addition, a second strategy was used to scan the CpG dense regions (including most CpG islands) for marker region identification. Reduced representation bisulfite sequencing (RRBS) was performed on the same panel of 75 samples. From the sequencing data generated by the two experimental approaches, regions where methylated haplotypes are detectible in primary tumor samples but present at a low level or non-detectible in plasma samples from normal individuals were searched for. This genome-wide screening identified a set of 5,301 candidate regions within blood UMRs, and 9,935 candidates from the RRBS data sets. Merging these two candidate lists yielded 11,901 candidate marker regions (listed in Table 4 provided below; the information in the rows and columns of this table has been provided as continuous text from which the rows and columns can be generated; in particular, rows of the table are separated by “;” and columns of each row are separated by “/”; Column 1 is Marker Genome Position, and Column 2 is Associated Genes) where tumor indicative haplotypes can be screened for in plasma samples. From the 30 cancer patient plasma, 5,871 regions were identified within which various levels of tumor indicative methylation haplotypes were detectible from patient's plasma. FIG. 7 shows an example at the promoter region of RhoB, which has been previously identified as a marker for cancer progression. Targeted methylation sequencing probe sets focusing on these 11,901 marker regions are being designed and optimized for examining larger sets of plasma samples from cancer patients and normal controls, in order to finalizing a specific set of markers with high sensitivity and specificity.

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

All publications, including patent documents and scientific articles, referred to in this application and the bibliography and attachments are incorporated by reference for the referenced materials and in their entireties for all purposes to the same extent as if each individual publication were individually incorporated by reference.

Citation of the above publications or documents is not intended as an admission that any of the foregoing is pertinent prior art, nor does it constitute any admission as to the contents or date of these publications or documents.

Although the present invention has been fully described in connection with embodiments thereof with reference to the accompanying drawings, it is to be noted that various changes and modifications will become apparent to those skilled in the art. Such changes and modifications are to be understood as being included within the scope of the present invention. The various embodiments of the invention should be understood that they have been presented by way of example only, and not by way of limitation. Likewise, the various diagrams may depict an example architectural or other configuration for the invention, which is done to aid in understanding the features and functionality that can be included in the invention. The invention is not restricted to the illustrated example architectures or configurations, but can be implemented using a variety of alternative architectures and configurations. Additionally, although the invention is described above in terms of various exemplary embodiments and implementations, it should be understood that the various features and functionality described in one or more of the individual embodiments are not limited in their applicability to the particular embodiment with which they are described. They instead can, be applied, alone or in some combination, to one or more of the other embodiments of the invention, whether or not such embodiments are described, and whether or not such features are presented as being a part of a described embodiment. Thus the breadth and scope of the invention should not be limited by any of the above-described exemplary embodiments.

Terms and phrases used in this document, and embodiments thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; and adjectives such as “conventional,” “traditional,” “normal,” “standard,” “known”, and terms of similar meaning, should not be construed as limiting the item described to a given time period, or to an item available as of a given time. But instead these terms should be read to encompass conventional, traditional, normal, or standard technologies that may be available, known now, or at any time in the future. Likewise, a group of items linked with the conjunction “and” should not be read as requiring that each and every one of those items be present in the grouping, but rather should be read as “and/or” unless apparent from the context or expressly stated otherwise. Similarly, a group of items linked with the conjunction “or” should not be read as requiring mutual exclusivity among that group, but rather should also be read as “and/or” unless it is apparent from the context or expressly stated otherwise. Furthermore, although items, elements or components of the invention may be described or claimed in the singular, the plural is contemplated to be within the scope thereof unless limitation to the singular is explicitly stated. For example, “at least one” may refer to a single or plural and is not limited to either. The presence of broadening words and phrases such as “one or more,” “at least,” “but not limited to”, or other like phrases in some instances shall not be read to mean that the narrower case is intended or required in instances where such broadening phrases may be absent.

### REFERENCES

The disclosures of each of the following references are incorporated herein by reference in their entireties:
U.S. Pat. No. 8,450,061 Quantification of a minority nucleic acid species
U.S. Pat. No. 8,586,313 DNA methylation markers based on epigenetic stem cell signatures in cancer
U.S. Pat. No. 8,563,242 Method for detecting chromosomal aneuploidy
U.S. Pat. No. 8,293,470 Non-invasive fetal genetic screening by digital analysis
U.S. Pat. No. 8,288,100 Methods for detecting fetal DNA in a plasma or serum sample from a pregnant woman
U.S. Pat. No. 8,026,067 Marker for prenatal diagnosis and monitoring
U.S. Pat. No. 8,110,361 DNA methylation markers associated with the CpG island methylator phenotype (CIMP) in human colorectal cancer
Chan et al. Size distributions of maternal and fetal DNA in maternal plasma. Clin Chem. (2004) 50:88-92
Chim et al. Systematic search for placental DNA-methylation markers on chromosome 21: toward a maternal plasma-based epigenetic test for fetal trisomy 21. Clin Chem (2008) 54:500-11
Chiu et al. Noninvasive prenatal diagnosis of fetal chromosomal aneuploidy by massively parallel genomic sequencing of DNA in maternal plasma. Proc Natl Acad Sci USA. (2008) 105:20458-63
Deng et al Targeted bisulfite sequencing reveals changes in DNA methylation associated with nuclear reprogramming. Nature Biotechnology (2009) 27:353-60
Diep et al., Library-free methylation sequencing with bisulfite padlock probes. Nature Methods, (2012) 9:270-272
Fan et al. Noninvasive diagnosis of fetal aneuploidy by shotgun sequencing DNA from maternal blood. Proc Natl Acad Sci USA. (2008) 105:16266-71
Fan et al. Non-invasive prenatal measurement of the fetal genome. Nature. (2012) 487:320-4
Kitzman et al. Noninvasive whole-genome sequencing of a human fetus. Sci Transl Med. (2012) 4:137 ra76
Kivioja et al. Counting absolute numbers of molecules using unique molecular identifiers. Nat Methods (2011) 9: 72-74
Johansson et al. Targeted resequencing of candidate genes using selector probes. Nucleic Acids Res. (2011) 39:e8. doi: 10.1093/nar/gkq1005
Komori et al. Application of microdroplet PCR for large-scale targeted bisulfite sequencing. Genome Research, (2011) 21: 1738-1745
Lun et al. Noninvasive prenatal methylomic analysis by genomewide bisulfite sequencing of maternal plasma DNA. Clin Chem. (2013) 59:1583-94
Papageorgiou et al. Fetal-specific DNA methylation ratio permits noninvasive prenatal diagnosis of trisomy 21. Nature Medicine (2010) 17:510-3
Papageorgiou & Patsalis. Non-invasive prenatal diagnosis of aneuploidies: new technologies and clinical applications. Genome Med. (2012) 4:46. doi: 10.1186/gm345
Plongthongkum et al. Advances in the profiling of DNA modifications: cytosine methylation and beyond. Nature Review Genetics (2014) 15:647-661
Poon et al. Differential DNA methylation between fetus and mother as a strategy for detecting fetal DNA in maternal plasma. Clin Chem. (2002) 48:35-41
Shoemaker et al. Allele-specific methylation is prevalent and is contributed by CpG-SNPs in the human genome. Genome Research (2010) 20:883-9

TABLE 1
chr1:58/chr1/968332/968434;chr1:59/chr1/968837/969693;chr1:68/chr1/1007436/1007622;chr1:135/chr1/11183
18/1118571;chr1:161/chr1/1182106/1182226;chr1:195/chr1/1290419/1290561;chr1:222/chr1/1475063/1475298;
chr1:224/chr1/1475644/1475960;chr1:225/chr1/1476094/1476254;chr1:250/chr1/1564730/1564962;chr1:252/c
hr1/1565418/1565749;chr1:253/chr1/1565856/1566233;chr1:254/chr1/1566540/1566945;chr1:255/chr1/156735
0/1567781;chr1:359/chr1/2005180/2005560;chr1:438/chr1/2137139/2137290;chr1:604/chr1/2461529/2461950;
chr1:624/chr1/2537228/2537597;chr1:645/chr1/2706335/2706746;chr1:691/chr1/2777169/2777476;chr1:831/ch
r1/2986787/2987055;chr1:971/chr1/3155183/3155453;chr1:1062/chr1/3310545/3310671;chr1:1191/chr1/34600
70/3460368;chr1:1196/chr1/3464790/3464968;chr1:1254/chr1/3567440/3567737;chr1:1360/chr1/4221485/4221
616;chr1:1408/chr1/4714362/4715427;chr1:1594/chr1/6126114/6126470;chr1:1690/chr1/6507256/6507490;chr
1:1739/chr1/7022040/7022295;chr1:1852/chr1/8277143/8277775;chr1:1974/chr1/10698623/10698890;chr1:220
5/chr1/12123554/12123787;chr1:2257/chr1/13839872/13840257;chr1:2371/chr1/16085288/16085860;chr1:256
6/chr1/18434320/18434751;chr1:3001/chr1/25255611/25255920;chr1:3004/chr1/25256824/25257156;chr1:315
0/chr1/27687074/27687463;chr1:3166/chr1/27895125/27895276;chr1:3196/chr1/29101687/29102039;chr1:320
9/chr1/29447872/29448068;chr1:3370/chr1/32827302/32827549;chr1:3371/chr1/32827824/32828016;chr1:340
0/chr1/33772282/33772445;chr1:3435/chr1/34642501/34642862;chr1:3496/chr1/36772176/36772239;chr1:353
9/chr1/37499683/37500013;chr1:3659/chr1/39957336/39957828;chr1:3709/chr1/41327514/41327843;chr1:376
7/chr1/43250819/43251073;chr1:3774/chr1/43533912/43534019;chr1:3805/chr1/44031559/44031834;chr1:384
5/chr1/44703923/44704146;chr1:3849/chr1/44872723/44872923;chr1:3851/chr1/44873173/44873509;chr1:385
6/chr1/44883215/44883459;chr1:3867/chr1/45082839/45083207;chr1:3916/chr1/46951318/46951628;chr1:395
3/chr1/47899597/47899892;chr1:3956/chr1/47904465/47904617;chr1:3957/chr1/47904733/47905407;chr1:405
1/chr1/50887085/50887175;chr1:4054/chr1/50888796/50889123;chr1:4108/chr1/53308608/53308688;chr1:417
7/chr1/54203422/54203828;chr1:4300/chr1/59041865/59042065;chr1:4313/chr1/60280395/60280812;chr1:436
3/chr1/63785293/63785618;chr1:4364/chr1/63785767/63786078;chr1:4370/chr1/63788767/63788919;chr1:437
1/chr1/63789497/63789728;chr1:4372/chr1/63789913/63790252;chr1:4393/chr1/65731506/65731781;chr1:448
6/chr1/77333924/77334021;chr1:4541/chr1/85358593/85358751;chr1:4547/chr1/85513805/85514226;chr1:456
6/chr1/86622552/86622812;chr1:4624/chr1/91183196/91183250;chr1:4640/chr1/91301627/91301840;chr1:482
4/chr1/110611514/110611653;chr1:4827/chr1/110612790/110613458;chr1:4838/chr1/110754580/110754871;c
hr1:4861/chr1/111216973/111217194;chr1:4908/chr1/113051685/113052056;chr1:4913/chr1/113286816/11328
7033;chr1:4957/chr1/115632038/115632104;chr1:5019/chr1/119526932/119527162;chr1:5025/chr1/119531997/
119532195;chr1:5027/chr1/119532784/119533069;chr1:5084/chr1/145562808/145562950;chr1:5137/chr1/150
522024/150522173;chr1:5180/chr1/151810485/151811028;chr1:5181/chr1/151811135/151811370;chr1:5284/c
hr1/154474585/154474743;chr1:5286/chr1/154475068/154475371;chr1:5370/chr1/156046537/156046781;chr1:
5568/chr1/161008704/161008977;chr1:5686/chr1/166916950/166917124;chr1:5882/chr1/180202395/18020263
7;chr1:6194/chr1/203044677/203045044;chr1:6195/chr1/203045172/203045278;chr1:6544/chr1/214153342/21
4153549;chr1:6573/chr1/217313043/217313068;chr1:6808/chr1/228135751/228135899;chr1:6929/chr1/228603
924/228604123;chr1:7109/chr1/234040481/234040885;chr1:7175/chr1/235813797/235814009;chr1:7209/chr1/
237205302/237205999;chr1:7210/chr1/237206266/237206511;chr1:7243/chr1/240161381/240161546;chr1:724
9/chr1/240256159/240256624;chr1:7360/chr1/246952274/246952510;chr1:7412/chr1/248020631/248020956;c
hr10:386/chr10/5734709/5734827;chr10:467/chr10/7450190/7450265;chr10:503/chr10/8076177/8076340;chr10:
519/chr10/8093964/8094141;chr10:524/chr10/8097588/8098078;chr10:583/chr10/11059664/11059932;chr10:5
84/chr10/11060158/11060314;chr10:585/chr10/11060435/11060512;chr10:586/chr10/11060629/11060741;chr1
0:629/chr10/11784414/11784802;chr10:784/chr10/16562043/16562368;chr10:801/chr10/17496096/17496544;c
hr10:808/chr10/18429329/18429517;chr10:846/chr10/21789240/21789471;chr10:848/chr10/21799129/2179941
9;chr10:859/chr10/22541851/22542024;chr10:861/chr10/22542250/22542567;chr10:872/chr10/22634175/2263
4324;chr10:905/chr10/23480697/23480903;chr10:906/chr10/23481050/23481303;chr10:950/chr10/26223206/2
6223425;chr10:958/chr10/26680735/26680960;chr10:983/chr10/28034556/28034873;chr10:1049/chr10/300240
81/30024742;chr10:1053/chr10/30025553/30026076;chr10:1066/chr10/30347775/30348116;chr10:1133/chr10/
35928850/35929333;chr10:1246/chr10/43600357/43600550;chr10:1271/chr10/43762277/43762357;chr10:1587/
chr10/52177823/52178170;chr10:1637/chr10/60936730/60937041;chr10:1956/chr10/73846982/73847427;chr1
0:1958/chr10/73847792/73848208;chr10:2022/chr10/77155863/77156184;chr10:2024/chr10/77156697/771568
85;chr10:2027/chr10/77158979/77159413;chr10:2138/chr10/81001956/81002247;chr10:2140/chr10/81002800/
81003671;chr10:2308/chr10/88122942/88123336;chr10:2309/chr10/88123453/88123631;chr10:2315/chr10/881
27133/88127346;chr10:2350/chr10/88728360/88728545;chr10:2358/chr10/90342871/90343138;chr10:2401/chr
10/94449496/94449797;chr10:2403/chr10/94450162/94450377;chr10:2407/chr10/94451603/94451827;chr10:2
424/chr10/94822606/94822908;chr10:2605/chr10/101281858/101282079;chr10:2667/chr10/102475409/102475
555;chr10:2670/chr10/102483796/102483870;chr10:2676/chr10/102507536/102507767;chr10:2698/chr10/1025
86406/102587188;chr10:2699/chr10/102587336/102587491;chr10:2700/chr10/102587613/102587830;chr10:27
05/chr10/102589101/102589250;chr10:2740/chr10/102899856/102900262;chr10:2761/chr10/102986953/10298
7206;chr10:2794/chr10/103538941/103538980;chr10:2795/chr10/103539125/103539367;chr10:2874/chr10/105
344239/105344746;chr10:2898/chr10/106028444/106028780;chr10:2911/chr10/106400918/106401322;chr10:2
949/chr10/108924401/108924769;chr10:2950/chr10/109674495/109674628;chr10:3131/chr10/118030732/1180
31118;chr10:3171/chr10/118893826/118893966;chr10:3172/chr10/118894072/118894477;chr10:3180/chr10/11
8899603/118899892;chr10:3225/chr10/119304986/119305061;chr10:3240/chr10/119494601/119494781;chr10:
3406/chr10/123922683/123923343;chr10:3443/chr10/124894607/124894870;chr10:3445/chr10/124896533/124
896937;chr10:3456/chr10/124909115/124909252;chr10:3457/chr10/124909538/124909673;chr10:3506/chr10/1
25651163/125651372;chr10:3516/chr10/125751136/125751180;chr10:3652/chr10/126840732/126840965;chr1
0:3710/chr10/128077241/128077431;chr10:3723/chr10/128995029/128995165;chr10:3746/chr10/129537031/1
29537354;chr10:4051/chr10/131757051/131757336;chr10:4054/chr10/131758037/131758094;chr10:4059/chr1
0/131763337/131763632;chr10:4068/chr10/131771168/131771281;chr10:4269/chr10/133109261/133110064;c
hr10:4270/chr10/133110185/133110486;chr10:4357/chr10/133796059/133796301;chr10:4449/chr10/13399912
7/133999350;chr10:4581/chr10/134598091/134598367;chr10:4582/chr10/134598534/134598860;chr10:4583/c
hr10/134599022/134599431;chr10:4584/chr10/134599546/134599708;chr10:4591/chr10/134601716/13460212
7;chr10:4732/chr10/134844677/134844889;chr10:4752/chr10/134862350/134862692;chr10:4868/chr10/135015
670/135016076;chr10:4898/chr10/135051810/135052097;chr10:4942/chr10/135192999/135193244;chr11:5/chr
11/280219/280472;chr11:69/chr11/504414/504539;chr11:75/chr11/536877/537026;chr11:98/chr11/626878/627
485;chr11:99/chr11/636907/637038;chr11:104/chr11/639571/639682;chr11:105/chr11/640059/640327;chr11:11
7/chr11/725713/725819;chr11:141/chr11/849371/850150;chr11:296/chr11/1358387/1358616;chr11:320/chr11/1
411785/1411841;chr11:389/chr11/1578093/1578391;chr11:461/chr11/1863865/1864192;chr11:550/chr11/1995
325/1995715;chr11:692/chr11/2236841/2237047;chr11:716/chr11/2291494/2291800;chr11:937/chr11/2871682/
2871856;chr11:1079/chr11/6651439/6651789;chr11:1094/chr11/7694886/7695108;chr11:1212/chr11/13030919/
13031244;chr11:1214/chr11/13032015/13032093;chr11:1427/chr11/20385280/20385481;chr11:1511/chr11/31
826831/31827113;chr11:1916/chr11/47416373/47416553;chr11:2133/chr11/61277120/61277327;chr11:2224/c
hr11/62212000/62212194;chr11:2279/chr11/63530253/63530615;chr11:2301/chr11/63767722/63768254;chr11:
2420/chr11/64428251/64428503;chr11:2460/chr11/64815091/64815281;chr11:2488/chr11/65246358/65246590
;chr11:2492/chr11/65313893/65314556;chr11:2506/chr11/65408370/65408651;chr11:2512/chr11/65553813/65
554042;chr11:2620/chr11/66885240/66886029;chr11:2667/chr11/67290667/67291004;chr11:2774/chr11/68145
282/68145588;chr11:3337/chr11/72295437/72295752;chr11:3411/chr11/74952729/74952943;chr11:3507/chr11/
76750930/76751074;chr11:3663/chr11/94134464/94134576;chr11:3664/chr11/94277706/94277933;chr11:371
0/chr11/100998745/100999022;chr11:3716/chr11/101454054/101454510;chr11:3841/chr11/112834094/112834
186;chr11:4166/chr11/119211245/119211650;chr11:4451/chr11/124790815/124791232;chr11:4458/chr11/1250
36487/125036720;chr11:4527/chr11/126286452/126286736;chr11:4678/chr11/130029438/130029860;chr11:46
86/chr11/130067674/130067921;chr11:4765/chr11/131780484/131780876;chr11:4766/chr11/131781151/13178
1293;chr12:51/chr12/679668/679979;chr12:156/chr12/2800446/2800603;chr12:157/chr12/2800841/2801019;ch
r12:307/chr12/4918889/4919144;chr12:417/chr12/6472994/6473162;chr12:428/chr12/6664523/6665446;chr12:
438/chr12/6882868/6883074;chr12:464/chr12/7592800/7592993;chr12:692/chr12/29302773/29302926;chr12:7
87/chr12/33592848/33592932;chr12:1084/chr12/50297555/50297973;chr12:1090/chr12/50354840/50355598;c
hr12:1112/chr12/51717749/51718204;chr12:1118/chr12/52115423/52115659;chr12:1125/chr12/52215177/5221
5837;chr12:1140/chr12/52301306/52301505;chr12:1169/chr12/52627047/52627272;chr12:1171/chr12/5265222
0/52652599;chr12:1258/chr12/54071000/54071194;chr12:1277/chr12/54338818/54338978;chr12:1282/chr12/5
4345659/54345965;chr12:1296/chr12/54355087/54355570;chr12:1313/chr12/54423458/54423615;chr12:1318/
chr12/54427107/54427293;chr12:1319/chr12/54427636/54427969;chr12:1321/chr12/54428361/54428699;chr1
2:1322/chr12/54441153/54441308;chr12:1427/chr12/57618711/57619110;chr12:1447/chr12/58013486/580137
13;chr12:1452/chr12/58021569/58021713;chr12:1457/chr12/58120237/58120457;chr12:1460/chr12/58129854/
58130183;chr12:1462/chr12/58131299/58131642;chr12:1463/chr12/58131768/58132114;chr12:1523/chr12/652
18551/65218900;chr12:1542/chr12/66123101/66123380;chr12:1613/chr12/72666032/72666342;chr12:1614/chr
12/72666808/72666908;chr12:1757/chr12/95941919/95942964;chr12:1797/chr12/99288962/99289161;chr12:1
858/chr12/104609797/104610080;chr12:1860/chr12/104697389/104697667;chr12:2428/chr12/117798950/1177
99086;chr12:2587/chr12/122016450/122016683;chr12:2890/chr12/127210935/127211316;chr12:2900/chr12/12
7630550/127630737;chr12:2942/chr12/128751878/128752114;chr12:2984/chr12/129337910/129338587;chr12:
3051/chr12/130388069/130388254;chr12:3052/chr12/130388435/130389012;chr12:3094/chr12/130646712/130
646945;chr12:3095/chr12/130647116/130647573;chr12:3097/chr12/130648070/130648407;chr12:3126/chr12/1
30909071/130909208;chr12:3619/chr12/133484853/133485161;chr13:45/chr13/20176135/20176478;chr13:85/
chr13/20875755/20876106;chr13:100/chr13/21295721/21296329;chr13:236/chr13/25320109/25320387;chr13:2
60/chr13/25744803/25744912;chr13:289/chr13/26625657/26626239;chr13:341/chr13/28498275/28498512;chr1
3:356/chr13/28549892/28550173;chr13:368/chr13/28674227/28674720;chr13:386/chr13/29107064/29107252;c
hr13:401/chr13/29394029/29394329;chr13:402/chr13/29394448/29394546;chr13:452/chr13/31480521/3148083
2;chr13:498/chr13/36045150/36045456;chr13:518/chr13/37005595/37005899;chr13:519/chr13/37006063/3700
6402;chr13:521/chr13/37248161/37248294;chr13:561/chr13/43148277/43148436;chr13:657/chr13/49794926/4
9795434;chr13:699/chr13/53174405/53174673;chr13:708/chr13/53420021/53420241;chr13:714/chr13/5342141
0/53421693;chr13:865/chr13/79170114/79170347;chr13:869/chr13/79176126/79176276;chr13:870/chr13/7917
6421/79176608;chr13:874/chr13/79177624/79177885;chr13:877/chr13/79183424/79183745;chr13:917/chr13/8
8324570/88324920;chr13:982/chr13/96294229/96294551;chr13:1053/chr13/100548190/100548513;chr13:1060/
chr13/100608036/100608461;chr13:1076/chr13/100641409/100642119;chr13:1082/chr13/100649576/1006497
99;chr13:1108/chr13/102568945/102569312;chr13:1173/chr13/109147863/109148154;chr13:1184/chr13/10979
2880/109793576;chr13:1196/chr13/110437010/110437493;chr13:1456/chr13/112721131/112721481;chr13:145
8/chr13/112721913/112722128;chr13:1461/chr13/112722982/112723292;chr13:1467/chr13/112727176/112727
256;chr13:1468/chr13/112727371/112727837;chr13:1471/chr13/112758691/112759038;chr13:1687/chr13/1136
99430/113699799;chr13:1700/chr13/113763898/113764152;chr13:1702/chr13/113764478/113764786;chr13:17
82/chr13/114523897/114524158;chr14:92/chr14/23834919/23834990;chr14:125/chr14/24045418/24045614;chr
14:157/chr14/24780119/24780742;chr14:164/chr14/24804123/24804352;chr14:212/chr14/27067157/27067372;
chr14:226/chr14/29235639/29235780;chr14:229/chr14/29236335/29236413;chr14:288/chr14/34419738/344198
20;chr14:312/chr14/36973222/36973454;chr14:322/chr14/36986664/36986872;chr14:323/chr14/36987031/369
87301;chr14:341/chr14/37053226/37053429;chr14:343/chr14/37116390/37116652;chr14:354/chr14/37127873/
37128139;chr14:363/chr14/37136018/37136294;chr14:369/chr14/37667110/37667286;chr14:379/chr14/380573
91/38057503;chr14:382/chr14/38061172/38061326;chr14:387/chr14/38064307/38064382;chr14:393/chr14/380
67496/38067697;chr14:397/chr14/38091399/38091606;chr14:411/chr14/38724544/38724978;chr14:454/chr14/
48144065/48144255;chr14:530/chr14/54421721/54421784;chr14:578/chr14/57264807/57265147;chr14:586/chr
14/57275874/57276073;chr14:625/chr14/59931122/59931193;chr14:626/chr14/59931439/59931719;chr14:627/
chr14/59931831/59932273;chr14:630/chr14/60097247/60097406;chr14:660/chr14/60981813/60981863;chr14:6
88/chr14/61746977/61747388;chr14:704/chr14/62217805/62217917;chr14:761/chr14/65209968/65210187;chr1
4:847/chr14/69095025/69095335;chr14:881/chr14/70038418/70038884;chr14:931/chr14/72399233/72399451;c
hr14:986/chr14/73958141/73958379;chr14:987/chr14/73958528/73958597;chr14:1032/chr14/75078243/750786
51;chr14:1137/chr14/77492338/77492788;chr14:1314/chr14/90528807/90528869;chr14:1402/chr14/92789822/
92789959;chr14:1433/chr14/93153549/93153846;chr14:1434/chr14/93154014/93154180;chr14:1435/chr14/931
54317/93154577;chr14:1600/chr14/95826752/95826905;chr14:1633/chr14/96343435/96343667;chr14:1682/chr
14/97499627/97499846;chr14:1768/chr14/99712739/99712922;chr14:1863/chr14/100626174/100626295;chr14:
1947/chr14/101193213/101193397;chr14:2038/chr14/101923687/101924030;chr14:2041/chr14/101925072/10
1925655;chr14:2078/chr14/102027172/102027654;chr14:2086/chr14/102057107/102057319;chr14:2099/chr14/
102172095/102172424;chr14:2140/chr14/103011141/103011360;chr14:2165/chr14/103395458/103395685;chr
14:2166/chr14/103395836/103396042;chr14:2167/chr14/103396311/103396470;chr14:2206/chr14/103674080/
103674215;chr14:2219/chr14/103739763/103739884;chr14:2285/chr14/104552067/104552238;chr14:2327/chr
14/104621931/104622185;chr14:2662/chr14/105553234/105553359;chr14:2727/chr14/105714589/105715025;
chr14:2758/chr14/105944309/105944557;chr14:2764/chr14/105953462/105953699;chr14:2833/chr14/1063205
09/106320797;chr15:201/chr15/27017335/27017654;chr15:202/chr15/27017905/27018210;chr15:203/chr15/27
018329/27018746;chr15:207/chr15/27112167/27112889;chr15:219/chr15/27216244/27216828;chr15:260/chr15/
28342020/28342440;chr15:264/chr15/28352543/28352930;chr15:275/chr15/29131048/29131532;chr15:428/ch
r15/31775783/31776145;chr15:523/chr15/37172508/37172792;chr15:578/chr15/40583657/40583807;chr15:628/
chr15/41787541/41788033;chr15:634/chr15/41794910/41795272;chr15:753/chr15/45479517/45479774;chr15:
839/chr15/52404573/52405032;chr15:867/chr15/53097569/53097777;chr15:935/chr15/58357220/58357437;chr
15:937/chr15/58357820/58358473;chr15:982/chr15/60296618/60296814;chr15:983/chr15/60296924/60297151;
chr15:1086/chr15/65116058/65116317;chr15:1162/chr15/66947282/66947434;chr15:1175/chr15/67325908/673
26118;chr15:1198/chr15/68119175/68119578;chr15:1200/chr15/68120353/68120577;chr15:1205/chr15/681221
34/68122469;chr15:1417/chr15/74425693/74426099;chr15:1477/chr15/75248484/75248822;chr15:1535/chr15/
76629026/76629162;chr15:1538/chr15/76630315/76630519;chr15:1553/chr15/76635110/76635575;chr15:1556/
chr15/76638497/76638855;chr15:1558/chr15/76639475/76639732;chr15:1830/chr15/83952113/83952430;chr1
5:1832/chr15/83953049/83953883;chr15:1841/chr15/84322323/84322476;chr15:1842/chr15/84322597/843229
93;chr15:1843/chr15/84323213/84323434;chr15:1864/chr15/85360568/85360738;chr15:2003/chr15/89922038/
89922499;chr15:2011/chr15/89950737/89951081;chr15:2015/chr15/89952453/89952699;chr15:2019/chr15/899
54842/89955001;chr15:2063/chr15/90543309/90543675;chr15:2091/chr15/91427469/91427788;chr15:2252/chr
15/95388002/95388253;chr15:2253/chr15/95388355/95388665;chr15:2393/chr15/99645065/99645300;chr15:2
467/chr15/100881844/100881966;chr15:2482/chr15/101267567/101267903;chr15:2490/chr15/101389973/1013
90107;chr15:2501/chr15/101513398/101513805;chr15:2502/chr15/101514012/101514212;chr15:2554/chr15/10
2029494/102030030;chr16:15/chr16/230316/230490;chr16:74/chr16/610501/610726;chr16:110/chr16/744110/7
44483;chr16:111/chr16/744849/745019;chr16:112/chr16/745122/745316;chr16:224/chr16/1024491/1024739;ch
r16:234/chr16/1034974/1035437;chr16:241/chr16/1040680/1041333;chr16:375/chr16/1203029/1203265;chr16:
393/chr16/1210829/1211001;chr16:549/chr16/1583820/1583986;chr16:660/chr16/2228968/2229216;chr16:679/
chr16/2286786/2286970;chr16:693/chr16/2392062/2392145;chr16:726/chr16/2835384/2835705;chr16:756/chr1
6/3016850/3017152;chr16:757/chr16/3017431/3017640;chr16:825/chr16/3550065/3550205;chr16:909/chr16/44
26241/4426409;chr16:1124/chr16/9107070/9107282;chr16:1299/chr16/12995804/12996073;chr16:1569/chr16/
22825470/22825885;chr16:1604/chr16/23847107/23847179;chr16:1606/chr16/23847512/23847788;chr16:1666/
chr16/25704123/25704271;chr16:1850/chr16/29888023/29888359;chr16:1968/chr16/31488749/31488924;chr1
6:2161/chr16/49316316/49316508;chr16:2379/chr16/51168338/51168672;chr16:2386/chr16/51185410/511855
42;chr16:2389/chr16/51186329/51186595;chr16:2445/chr16/54324187/54324470;chr16:2474/chr16/54970258/
54970523;chr16:2476/chr16/54971007/54971225;chr16:2477/chr16/54971586/54971790;chr16:2511/chr16/553
58351/55358566;chr16:2526/chr16/55543053/55543209;chr16:2542/chr16/56228417/56228577;chr16:2582/chr
16/56669402/56669552;chr16:2668/chr16/58058589/58058926;chr16:2827/chr16/66613096/66613334;chr16:2
882/chr16/67428252/67428716;chr16:2907/chr16/67687484/67687638;chr16:2915/chr16/67918680/67918901;
chr16:2924/chr16/68118640/68118759;chr16:3092/chr16/73096889/73097141;chr16:3098/chr16/73100299/731
00575;chr16:3215/chr16/77468458/77469028;chr16:3585/chr16/85320512/85320774;chr16:3821/chr16/865420
53/86542112;chr16:3825/chr16/86544652/86544928;chr16:3863/chr16/86962690/86962924;chr16:3925/chr16/
87636491/87636626;chr16:4071/chr16/88442893/88443159;chr16:4109/chr16/88496740/88497351;chr16:4179/
chr16/88600726/88601095;chr16:4191/chr16/88700818/88701421;chr17:176/chr17/1173782/1174273;chr17:2
70/chr17/1958883/1959132;chr17:271/chr17/1959264/1959436;chr17:273/chr17/1959751/1960008;chr17:274/c
hr17/1960198/1960286;chr17:275/chr17/1960474/1960778;chr17:276/chr17/1960883/1961088;chr17:279/chr17/
1962230/1962612;chr17:309/chr17/2627584/2627798;chr17:534/chr17/4802300/4802981;chr17:537/chr17/480
4078/4804454;chr17:550/chr17/4981549/4981823;chr17:551/chr17/5000936/5001149;chr17:658/chr17/689940
3/6899645;chr17:715/chr17/7287208/7287338;chr17:859/chr17/8926559/8926812;chr17:965/chr17/10101448/1
0102264;chr17:1003/chr17/11143844/11144748;chr17:1004/chr17/11144852/11144922;chr17:1078/chr17/1287
7459/12877650;chr17:1098/chr17/13504770/13504982;chr17:1113/chr17/14212473/14212588;chr17:1218/chr1
7/17399137/17399419;chr17:1243/chr17/17625795/17626075;chr17:1256/chr17/17713331/17713585;chr17:13
16/chr17/19627925/19628208;chr17:1522/chr17/27038686/27038906;chr17:1552/chr17/27940338/27940509;c
hr17:1558/chr17/28088300/28088606;chr17:1606/chr17/30244207/30244357;chr17:1676/chr17/32484214/3248
4265;chr17:1813/chr17/35299444/35299600;chr17:1814/chr17/35299966/35300260;chr17:1855/chr17/3666664
1/36667136;chr17:1867/chr17/36718155/36718367;chr17:1933/chr17/38334152/38334396;chr17:1937/chr17/3
8473031/38473221;chr17:2060/chr17/40822042/40822411;chr17:2173/chr17/43037309/43037504;chr17:2175/
chr17/43044869/43045038;chr17:2176/chr17/43045266/43045371;chr17:2190/chr17/43198566/43198759;chr1
7:2281/chr17/44896870/44897012;chr17:2301/chr17/45500826/45500865;chr17:2307/chr17/45786312/457865
80;chr17:2349/chr17/46604392/46604639;chr17:2375/chr17/46710990/46711510;chr17:2385/chr17/46796374/
46796498;chr17:2417/chr17/47073328/47073388;chr17:2514/chr17/48194835/48195141;chr17:2535/chr17/485
46120/48546321;chr17:2537/chr17/48546623/48546755;chr17:2653/chr17/54755991/54756198;chr17:2750/chr
17/56564884/56565077;chr17:2778/chr17/58227398/58227787;chr17:2797/chr17/59473504/59473707;chr17:2
799/chr17/59474247/59474741;chr17:2810/chr17/59482595/59482700;chr17:2816/chr17/59488424/59488634;
chr17:2823/chr17/59529265/59529626;chr17:2827/chr17/59532018/59532148;chr17:2890/chr17/61778085/617
78234;chr17:2925/chr17/62775015/62775245;chr17:3065/chr17/70112034/70112136;chr17:3421/chr17/730737
00/73073967;chr17:3473/chr17/73584040/73584287;chr17:3474/chr17/73584485/73584937;chr17:3502/chr17/
73749654/73750176;chr17:3547/chr17/74136170/74136296;chr17:3789/chr17/76127483/76127750;chr17:4130/
chr17/77776566/77776679;chr17:4139/chr17/77788633/77789351;chr17:4140/chr17/77789528/77789833;chr1
7:4148/chr17/77814190/77814493;chr17:4170/chr17/77916767/77916996;chr17:4420/chr17/79314980/793151
31;chr17:4422/chr17/79315509/79315667;chr17:4511/chr17/79615136/79615382;chr17:4566/chr17/79919577/
79919914;chr17:4657/chr17/80332838/80332948;chr18:75/chr18/4454691/4454899;chr18:77/chr18/4455272/4
455561;chr18:91/chr18/5543158/5543430;chr18:92/chr18/5543547/5543801;chr18:97/chr18/5629826/5630124;
chr18:105/chr18/5890352/5890848;chr18:106/chr18/5891003/5891336;chr18:280/chr18/11751632/11752127;c
hr18:430/chr18/19751266/19751424;chr18:431/chr18/19751535/19751774;chr18:465/chr18/21269952/2127012
6;chr18:487/chr18/22929719/22929926;chr18:503/chr18/24130414/24130644;chr18:566/chr18/31020511/3102
0915;chr18:577/chr18/32073831/32073907;chr18:585/chr18/32847334/32847597;chr18:608/chr18/34823894/3
4823936;chr18:610/chr18/34833555/34833958;chr18:637/chr18/35065146/35065516;chr18:707/chr18/4341770
7/43417871;chr18:723/chr18/43913755/43914155;chr18:763/chr18/44336450/44337012;chr18:765/chr18/4433
7842/44338098;chr18:768/chr18/44526700/44527026;chr18:776/chr18/44773967/44774201;chr18:891/chr18/4
7794717/47795022;chr18:966/chr18/53447475/53447777;chr18:989/chr18/55019776/55020040;chr18:994/chr1
8/55094849/55095353;chr18:1044/chr18/56940171/56940565;chr18:1046/chr18/56941245/56941557;chr18:10
54/chr18/57364050/57364448;chr18:1125/chr18/63417896/63418336;chr18:1140/chr18/67068114/67068592;c
hr18:1150/chr18/70209386/70209493;chr18:1153/chr18/70534316/70534426;chr18:1155/chr18/70534732/7053
5220;chr18:1223/chr18/72916657/72916830;chr18:1256/chr18/73628238/73628494;chr18:1360/chr18/7496235
8/74962692;chr18:1609/chr18/76739081/76739420;chr18:1610/chr18/76739575/76739634;chr18:1611/chr18/7
6739745/76740101;chr18:1613/chr18/76740404/76741072;chr18:1633/chr18/76828918/76829011;chr18:1804/
chr18/77389255/77389513;chr18:1835/chr18/77548048/77548351;chr19:56/chr19/518850/519279;chr19:69/chr
19/591646/591765;chr19:158/chr19/863044/863220;chr19:211/chr19/1041941/1042228;chr19:267/chr19/11688
50/1169089;chr19:298/chr19/1256998/1257181;chr19:322/chr19/1295636/1295874;chr19:325/chr19/1302544/1
302808;chr19:362/chr19/1457047/1457373;chr19:367/chr19/1467273/1468805;chr19:368/chr19/1468943/1469
945;chr19:369/chr19/1470071/1470383;chr19:372/chr19/1470953/1471094;chr19:392/chr19/1524150/1524433;
chr19:452/chr19/1762506/1762627;chr19:550/chr19/2250706/2251061;chr19:551/chr19/2251235/2251610;chr1
9:553/chr19/2251950/2252313;chr19:562/chr19/2282567/2282729;chr19:566/chr19/2290272/2290630;chr19:56
7/chr19/2290868/2291315;chr19:759/chr19/3435252/3435460;chr19:830/chr19/3786221/3786583;chr19:918/ch
r19/4328745/4328968;chr19:1180/chr19/6744856/6745014;chr19:1417/chr19/9473598/9473868;chr19:1577/chr
19/11689685/11689880;chr19:1690/chr19/13207431/13207610;chr19:1696/chr19/13209705/13210566;chr19:1
699/chr19/13215258/13215542;chr19:1717/chr19/13409455/13409643;chr19:1722/chr19/13616957/13617158;
chr19:1748/chr19/14089652/14089812;chr19:1749/chr19/14089930/14090019;chr19:1807/chr19/14583062/145
83153;chr19:1881/chr19/15580445/15580775;chr19:1946/chr19/16436338/16436566;chr19:1986/chr19/170085
19/17008585;chr19:1987/chr19/17008699/17008820;chr19:2029/chr19/17434486/17434656;chr19:2031/chr19/
17439019/17439359;chr19:2032/chr19/17439537/17439938;chr19:2104/chr19/17983666/17983839;chr19:2193/
chr19/18771109/18771428;chr19:2231/chr19/18980718/18980911;chr19:2257/chr19/19335831/19335974;chr1
9:2488/chr19/29284576/29284929;chr19:2540/chr19/30016929/30017451;chr19:2639/chr19/30866025/308663
28;chr19:2725/chr19/31839566/31839837;chr19:2730/chr19/31842481/31842673;chr19:2895/chr19/34112730/
34113145;chr19:3094/chr19/36246642/36246780;chr19:3157/chr19/37096038/37096487;chr19:3205/chr19/380
42198/38042356;chr19:3278/chr19/38886200/38886855;chr19:3321/chr19/39086879/39087135;chr19:3462/chr
19/40729228/40729385;chr19:3497/chr19/41106002/41106351;chr19:3503/chr19/41119409/41119669;chr19:3
504/chr19/41119802/41119956;chr19:3621/chr19/42703468/42703974;chr19:3765/chr19/45900030/45900160;
chr19:3827/chr19/46387865/46387953;chr19:3833/chr19/46443412/46443636;chr19:3840/chr19/46518378/465
18642;chr19:3873/chr19/46996839/46997167;chr19:3890/chr19/47152991/47153218;chr19:3923/chr19/479103
79/47910558;chr19:3997/chr19/48946166/48946452;chr19:3998/chr19/48946629/48946707;chr19:3999/chr19/
48946849/48947361;chr19:4004/chr19/48983719/48983942;chr19:4075/chr19/49575475/49576106;chr19:4173/
chr19/50393577/50393796;chr19:4204/chr19/50789807/50789973;chr19:4216/chr19/50861847/50861922;chr1
9:4257/chr19/51162254/51162427;chr19:4357/chr19/52222924/52223142;chr19:4369/chr19/52552120/525522
33;chr19:4653/chr19/55598064/55598375;chr19:4654/chr19/55598495/55598810;chr19:4708/chr19/55997744/
55998019;chr19:4796/chr19/56988664/56988963;chr19:4808/chr19/57050288/57050428;chr19:4862/chr19/580
38585/58038923;chr19:4874/chr19/58238739/58238987;chr19:4895/chr19/58545590/58545651;chr19:4904/chr
19/58570589/58570809;chr19:4915/chr19/58609526/58609712;chr19:4937/chr19/58867803/58867983;chr19:4
939/chr19/58868255/58868372;chr19:4944/chr19/58907637/58908323;chr2:38/chr2/468234/468423;chr2:117/c
hr2/946224/946289;chr2:118/chr2/946567/946916;chr2:257/chr2/1747033/1747305;chr2:259/chr2/1748008/174
8396;chr2:525/chr2/3751257/3751367;chr2:597/chr2/5832048/5832283;chr2:598/chr2/5832526/5832655;chr2:5
99/chr2/5832847/5833282;chr2:605/chr2/5836992/5837352;chr2:667/chr2/7571477/7571576;chr2:741/chr2/881
6718/8817071;chr2:1181/chr2/24232789/24233349;chr2:1220/chr2/25438872/25439138;chr2:1230/chr2/25499
767/25500398;chr2:1263/chr2/26407561/26407743;chr2:1264/chr2/26408043/26408198;chr2:1346/chr2/27530
528/27530844;chr2:1376/chr2/29033698/29033897;chr2:1519/chr2/39187238/39187543;chr2:1550/chr2/42328
679/42328778;chr2:1560/chr2/42720447/42720653;chr2:1706/chr2/47596849/47596941;chr2:1768/chr2/54086
982/54087153;chr2:1902/chr2/63274615/63275188;chr2:1903/chr2/63275509/63275877;chr2:1921/chr2/63282
867/63283013;chr2:1925/chr2/63286164/63286358;chr2:1926/chr2/63286748/63287082;chr2:1990/chr2/66672
431/66672704;chr2:1992/chr2/66673076/66673326;chr2:2063/chr2/71115961/71116352;chr2:2164/chr2/73147
789/73147852;chr2:2215/chr2/74725139/74725244;chr2:2224/chr2/74742151/74742324;chr2:2227/chr2/74743
145/74743455;chr2:2233/chr2/74875255/74875477;chr2:2240/chr2/75147103/75147307;chr2:2322/chr2/85361
529/85361628;chr2:2426/chr2/88751816/88751942;chr2:2432/chr2/89064997/89065076;chr2:2631/chr2/99438
893/99439368;chr2:2749/chr2/105459152/105459378;chr2:2750/chr2/105459575/105459857;chr2:2758/chr2/1
05472597/105472712;chr2:2852/chr2/107502908/107503421;chr2:3000/chr2/110372422/110372665;chr2:3031/
chr2/111876685/111877171;chr2:3049/chr2/112939243/112939539;chr2:3074/chr2/114034675/114034908;chr
2:3075/chr2/114035058/114035443;chr2:3107/chr2/115918938/115919206;chr2:3108/chr2/115919425/115919
830;chr2:3447/chr2/127413996/127414108;chr2:3708/chr2/131721099/131721347;chr2:3710/chr2/131721665/
131721866;chr2:3718/chr2/131797507/131797859;chr2:3847/chr2/145281979/145282041;chr2:3916/chr2/1543
34914/154335184;chr2:3925/chr2/154728977/154729082;chr2:3996/chr2/162279888/162280152;chr2:3997/chr
2/162280416/162280604;chr2:3999/chr2/162283603/162283782;chr2:4079/chr2/171570190/171570470;chr2:4
122/chr2/172947914/172948183;chr2:4124/chr2/172952084/172952153;chr2:4127/chr2/172954739/172954982;
chr2:4131/chr2/172964889/172965295;chr2:4132/chr2/172965492/172965647;chr2:4172/chr2/174877642/174
877777;chr2:4182/chr2/175199555/175199921;chr2:4183/chr2/175200441/175200709;chr2:4186/chr2/1752012
95/175201359;chr2:4187/chr2/175201542/175201775;chr2:4189/chr2/175202246/175202568;chr2:4191/chr2/1
75204947/175205526;chr2:4214/chr2/176944847/176945137;chr2:4215/chr2/176945269/176945581;chr2:4217/
chr2/176946952/176947284;chr2:4218/chr2/176948022/176948199;chr2:4224/chr2/176957629/176957914;chr
2:4233/chr2/176972376/176972742;chr2:4245/chr2/176981212/176981376;chr2:4251/chr2/176987225/176987
465;chr2:4252/chr2/176987662/176987970;chr2:4262/chr2/176994622/176995070;chr2:4263/chr2/176995270/
176995331;chr2:4268/chr2/177016417/177016730;chr2:4269/chr2/177017078/177017331;chr2:4284/chr2/1770
36685/177036813;chr2:4287/chr2/177042999/177043266;chr2:4288/chr2/177053703/177053906;chr2:4449/chr
2/200327259/200327423;chr2:4451/chr2/200328685/200329029;chr2:4572/chr2/209271383/209271602;chr2:4
739/chr2/219866132/219866437;chr2:4772/chr2/220312976/220313971;chr2:4784/chr2/220361467/220361644;
chr2:4945/chr2/231693108/231693406;chr2:5038/chr2/233285369/233285625;chr2:5206/chr2/236579469/236
579780;chr2:5207/chr2/236579970/236580117;chr2:5231/chr2/237072391/237072610;chr2:5240/chr2/2370785
15/237078746;chr2:5244/chr2/237081427/237081536;chr2:5245/chr2/237081686/237082281;chr2:5386/chr2/2
39072474/239073018;chr2:5420/chr2/239755654/239756024;chr2:5862/chr2/242498266/242498764;chr20:16/
chr20/590749/590967;chr20:54/chr20/825407/825542;chr20:55/chr20/825845/826228;chr20:62/chr20/982799/9
82891;chr20:90/chr20/1784369/1784547;chr20:134/chr20/2673000/2673201;chr20:136/chr20/2674155/267469
6;chr20:144/chr20/2732645/2732726;chr20:476/chr20/20345958/20346166;chr20:516/chr20/21081845/210820
94;chr20:524/chr20/21376337/21376702;chr20:525/chr20/21377129/21377450;chr20:534/chr20/21491346/214
91616;chr20:555/chr20/21503174/21503454;chr20:620/chr20/23029232/23029549;chr20:732/chr20/25128964/
25129184;chr20:752/chr20/25565669/25565825;chr20:764/chr20/26189103/26189330;chr20:765/chr20/261896
86/26189956;chr20:924/chr20/32254422/32254923;chr20:925/chr20/32255610/32255936;chr20:946/chr20/328
56719/32857151;chr20:1038/chr20/36012804/36012923;chr20:1322/chr20/43727095/43727357;chr20:1381/chr
20/44686066/44686303;chr20:1728/chr20/50721671/50721988;chr20:1810/chr20/55201357/55201450;chr20:1
812/chr20/55202160/55202358;chr20:1816/chr20/55205780/55206229;chr20:1833/chr20/55500302/55500440;
chr20:1878/chr20/55965169/55965524;chr20:1991/chr20/57224597/57224798;chr20:2023/chr20/57464249/574
64384;chr20:2056/chr20/57766396/57766794;chr20:2210/chr20/59826978/59828340;chr20:2212/chr20/598286
48/59828962;chr20:2677/chr20/61638107/61638180;chr20:2719/chr20/61703466/61703709;chr20:2928/chr20/
62119619/62119922;chr20:2966/chr20/62272683/62272984;chr20:2983/chr20/62369273/62369503;chr20:3023/
chr20/62463400/62463622;chr21:56/chr21/19191198/19191363;chr21:59/chr21/19617176/19617394;chr21:10
0/chr21/27945234/27945553;chr21:102/chr21/28337870/28338039;chr21:158/chr21/32931204/32931770;chr21:
214/chr21/34442257/34442594;chr21:324/chr21/38076709/38077206;chr21:331/chr21/38080387/38080550;ch
r21:333/chr21/38081193/38081665;chr21:384/chr21/40032525/40032679;chr21:448/chr21/42218760/42218851;
chr21:666/chr21/44037202/44037470;chr21:861/chr21/45577552/45577873;chr21:948/chr21/45875391/45875
681;chr21:1054/chr21/46294322/46294496;chr21:1182/chr21/46847897/46848190;chr21:1253/chr21/47010153/
47010408;chr21:1265/chr21/47062088/47062752;chr22:14/chr22/17082524/17082786;chr22:15/chr22/170830
62/17083207;chr22:34/chr22/17566540/17566737;chr22:141/chr22/19511876/19512252;chr22:161/chr22/1971
0880/19711327;chr22:162/chr22/19711431/19711681;chr22:163/chr22/19711794/19712089;chr22:294/chr22/2
0779588/20779897;chr22:295/chr22/20780367/20780437;chr22:299/chr22/20784958/20785119;chr22:369/chr2
2/22862863/22863219;chr22:531/chr22/26565386/26565502;chr22:720/chr22/30661932/30662102;chr22:826/c
hr22/33454259/33454345;chr22:1094/chr22/38478672/38478861;chr22:1246/chr22/40045631/40045721;chr22:
1248/chr22/40057992/40058286;chr22:1288/chr22/40391230/40391446;chr22:1308/chr22/41048383/41048593;
chr22:1367/chr22/42679480/42679916;chr22:1515/chr22/43807507/43807746;chr22:1790/chr22/46263114/46
263402;chr22:1823/chr22/46368124/46368554;chr22:1842/chr22/46460277/46460482;chr22:1851/chr22/46474
655/46474921;chr22:1852/chr22/46475686/46476597;chr22:1858/chr22/46480891/46481121;chr22:1865/chr22/
46484646/46484897;chr22:1873/chr22/46500069/46500450;chr22:1894/chr22/46658743/46659224;chr22:192
1/chr22/46932357/46932650;chr22:2146/chr22/48884960/48885529;chr22:2148/chr22/48885838/48885907;chr
22:2149/chr22/48886022/48886204;chr22:2205/chr22/48970924/48971532;chr22:2206/chr22/48971680/48971
758;chr22:2208/chr22/48972136/48972219;chr22:2210/chr22/48972471/48972766;chr22:2526/chr22/49969183/
49969655;chr22:2676/chr22/50496583/50496971;chr22:2703/chr22/50623290/50623612;chr22:2705/chr22/50
629027/50629294;chr22:2722/chr22/50706290/50706502;chr22:2727/chr22/50737741/50738598;chr22:2759/c
hr22/50982723/50982902;chr22:2764/chr22/50987096/50987423;chr22:2774/chr22/51042646/51042974;chr22:
2791/chr22/51135620/51136021;chr22:2792/chr22/51136180/51136408;chr3:5/chr3/238716/239006;chr3:9/chr
3/239869/240109;chr3:20/chr3/2140435/2140827;chr3:138/chr3/8809715/8810151;chr3:160/chr3/9595199/959
5395;chr3:161/chr3/9595503/9595598;chr3:220/chr3/10857721/10858054;chr3:333/chr3/13114797/13115104;c
hr3:392/chr3/13324029/13324234;chr3:393/chr3/13324348/13324419;chr3:394/chr3/13324674/13324740;chr3:
431/chr3/13590641/13590973;chr3:649/chr3/22414078/22414264;chr3:761/chr3/32858959/32859171;chr3:762/
chr3/32859285/32859417;chr3:823/chr3/37494130/37494424;chr3:1010/chr3/45187328/45188139;chr3:1095/c
hr3/47050867/47051151;chr3:1154/chr3/48693701/48693851;chr3:1158/chr3/48699011/48699376;chr3:1321/c
hr3/51990188/51990359;chr3:1502/chr3/58572710/58572807;chr3:1611/chr3/65342126/65342389;chr3:1613/c
hr3/65534763/65534926;chr3:1614/chr3/65535125/65535274;chr3:1771/chr3/73673729/73673951;chr3:2134/c
hr3/123167020/123167300;chr3:2172/chr3/124860671/124860898;chr3:2181/chr3/125677058/125677226;chr3:
2382/chr3/128210649/128210888;chr3:2383/chr3/128211117/128211451;chr3:2384/chr3/128211603/12821176
9;chr3:2385/chr3/128211992/128212336;chr3:2394/chr3/128274052/128274308;chr3:2434/chr3/128764607/12
8764836;chr3:2471/chr3/129693499/129693954;chr3:2518/chr3/133393269/133393622;chr3:2544/chr3/134031
856/134031958;chr3:2629/chr3/138658297/138658579;chr3:2635/chr3/138663977/138664141;chr3:2636/chr3/
138664383/138664701;chr3:2641/chr3/138679293/138679565;chr3:2753/chr3/147111300/147111702;chr3:276
8/chr3/147127565/147127676;chr3:2770/chr3/147128083/147128529;chr3:2880/chr3/156544017/156544403;c
hr3:2897/chr3/157821233/157821536;chr3:2978/chr3/170137345/170137581;chr3:3063/chr3/179754873/17975
5086;chr3:3077/chr3/181413201/181413421;chr3:3168/chr3/184301671/184301892;chr3:3248/chr3/186490643/
186490790;chr3:3332/chr3/192232962/192233094;chr3:3517/chr3/196255632/196256011;chr4:5/chr4/107278/
107896;chr4:15/chr4/330292/330715;chr4:24/chr4/568681/569047;chr4:26/chr4/569866/570063;chr4:56/chr4/6
75809/676045;chr4:62/chr4/682750/682943;chr4:109/chr4/967617/967716;chr4:122/chr4/996175/996400;chr4:
123/chr4/996539/997292;chr4:158/chr4/1164982/1165095;chr4:198/chr4/1396773/1396923;chr4:202/chr4/1398
715/1398990;chr4:205/chr4/1400015/1400112;chr4:207/chr4/1400340/1400870;chr4:241/chr4/1537691/153814
8;chr4:391/chr4/2042259/2042655;chr4:470/chr4/2820092/2820468;chr4:616/chr4/3734136/3734361;chr4:637/
chr4/3769190/3769411;chr4:759/chr4/5709985/5710450;chr4:773/chr4/5894116/5894463;chr4:774/chr4/58946
33/5894812;chr4:1265/chr4/8859938/8860397;chr4:1270/chr4/8862812/8863143;chr4:1272/chr4/8863527/8863
856;chr4:1277/chr4/8873073/8873773;chr4:1280/chr4/8874535/8874713;chr4:1288/chr4/8894958/8895231;chr
4:1290/chr4/8895776/8895964;chr4:1403/chr4/13543753/13544277;chr4:1405/chr4/13545523/13545940;chr4:1
475/chr4/20254057/20254239;chr4:1479/chr4/20255368/20255524;chr4:1481/chr4/20256577/20256841;chr4:1
487/chr4/21950296/21950626;chr4:1509/chr4/24801526/24801848;chr4:1578/chr4/30723513/30723855;chr4:1
602/chr4/37245582/37245836;chr4:1604/chr4/37246700/37247015;chr4:1664/chr4/40858965/40859293;chr4:1
685/chr4/41869200/41869413;chr4:1701/chr4/42153412/42153532;chr4:1702/chr4/42153633/42153881;chr4:1
726/chr4/44449743/44449971;chr4:1727/chr4/44450177/44450741;chr4:1747/chr4/48485289/48485589;chr4:1
750/chr4/48492930/48493306;chr4:2032/chr4/81123904/81124121;chr4:2076/chr4/85403941/85404111;chr4:2
081/chr4/85414337/85414457;chr4:2084/chr4/85418583/85419049;chr4:2130/chr4/91048946/91049201;chr4:2
250/chr4/111542093/111542473;chr4:2332/chr4/122301713/122301962;chr4:2398/chr4/135122689/135122873;
chr4:2434/chr4/140656667/140656782;chr4:2508/chr4/147576223/147576329;chr4:2602/chr4/155411494/155
411850;chr4:2610/chr4/155665036/155665292;chr4:2621/chr4/158141657/158141754;chr4:2647/chr4/1642648
53/164265192;chr4:2703/chr4/172734392/172734591;chr4:2712/chr4/174427891/174428278;chr4:2716/chr4/1
74430554/174430793;chr4:2973/chr4/188916495/188916865;chr4:2981/chr4/189203770/189204020;chr5:91/c
hr5/528469/528888;chr5:221/chr5/1059174/1059425;chr5:501/chr5/1876921/1877089;chr5:504/chr5/1878577/1
878830;chr5:506/chr5/1882136/1882419;chr5:511/chr5/1886077/1886379;chr5:514/chr5/1887179/1887546;chr
5:535/chr5/1931287/1931444;chr5:703/chr5/2750517/2750654;chr5:705/chr5/2750949/2751854;chr5:706/chr5/
2751982/2752120;chr5:827/chr5/3594520/3594777;chr5:829/chr5/3595432/3595849;chr5:830/chr5/3596102/35
96347;chr5:838/chr5/3600753/3601025;chr5:839/chr5/3602351/3602713;chr5:1055/chr5/7849946/7850250;chr
5:1172/chr5/15500117/15500570;chr5:1190/chr5/16179857/16180165;chr5:1380/chr5/37840363/37840529;chr
5:1410/chr5/40681840/40682069;chr5:1434/chr5/42952112/42952433;chr5:1453/chr5/43397230/43397305;chr
5:1488/chr5/50695241/50695332;chr5:1602/chr5/59189058/59189188;chr5:1614/chr5/60922159/60922296;chr
5:1664/chr5/66462080/66462646;chr5:1679/chr5/68710972/68711443;chr5:1710/chr5/72594127/72594501;chr
5:1718/chr5/72677195/72677774;chr5:1719/chr5/72678029/72678365;chr5:1725/chr5/72732454/72732774;chr
5:1726/chr5/72732910/72733012;chr5:1789/chr5/76373091/76373686;chr5:1812/chr5/77147650/77147872;chr
5:1826/chr5/77806046/77806301;chr5:1850/chr5/79331135/79331229;chr5:1861/chr5/79865834/79866099;chr
5:1864/chr5/80256075/80256312;chr5:1926/chr5/92906273/92906757;chr5:1949/chr5/92939837/92940177;chr
5:2048/chr5/112538998/112539281;chr5:2080/chr5/115152485/115152616;chr5:2195/chr5/127873297/127873
552;chr5:2196/chr5/127873806/127873980;chr5:2198/chr5/127874478/127874705;chr5:2246/chr5/132083377/
132083472;chr5:2252/chr5/132149891/132150239;chr5:2253/chr5/132158700/132159057;chr5:2254/chr5/1321
61114/132161807;chr5:2324/chr5/134363537/134364144;chr5:2325/chr5/134364290/134364693;chr5:2354/chr
5/134825791/134825912;chr5:2364/chr5/134871102/134871337;chr5:2392/chr5/135266129/135266577;chr5:2
417/chr5/136834051/136834286;chr5:2450/chr5/138729925/138730147;chr5:2489/chr5/139228001/139228242;
chr5:2522/chr5/140306172/140306444;chr5:2559/chr5/140871113/140871639;chr5:2560/chr5/140871959/140
872219;chr5:2729/chr5/149546649/149546969;chr5:2932/chr5/158737656/158737744;chr5:2942/chr5/1593991
00/159399509;chr5:3113/chr5/170289023/170289638;chr5:3116/chr5/170735469/170735730;chr5:3119/chr5/1
70736868/170737281;chr5:3128/chr5/170847581/170847717;chr5:3129/chr5/170847951/170848081;chr5:3133/
chr5/170878017/170878251;chr5:3195/chr5/172111150/172111287;chr5:3298/chr5/174158852/174159299;chr
5:3347/chr5/175223950/175224441;chr5:3395/chr5/176023882/176024005;chr5:3481/chr5/176544032/176544
187;chr5:3484/chr5/176559854/176560001;chr5:3532/chr5/177540239/177540402;chr5:3534/chr5/177540722/
177540787;chr5:3644/chr5/178003699/178004204;chr5:3649/chr5/178016984/178017177;chr5:3695/chr5/1784
21519/178422143;chr5:3837/chr5/178957437/178957597;chr5:3858/chr5/179243878/179244320;chr5:3916/chr
5/180018132/180018484;chr5:3993/chr5/180486204/180486792;chr6:21/chr6/391900/392409;chr6:22/chr6/392
959/393124;chr6:86/chr6/1312356/1312594;chr6:92/chr6/1313904/1314000;chr6:93/chr6/1314101/1314219;chr
6:95/chr6/1378476/1379106;chr6:98/chr6/1383709/1383859;chr6:99/chr6/1384180/1384444;chr6:105/chr6/139
3049/1393301;chr6:107/chr6/1393757/1394089;chr6:138/chr6/1614406/1615083;chr6:144/chr6/1620404/16205
12;chr6:245/chr6/4775997/4776187;chr6:256/chr6/5086329/5086642;chr6:279/chr6/6003683/6004349;chr6:317/
chr6/7051662/7051765;chr6:380/chr6/10381969/10382321;chr6:384/chr6/10384975/10385279;chr6:458/chr6/1
2749941/12750364;chr6:596/chr6/19837421/19837743;chr6:789/chr6/27725175/27725416;chr6:852/chr6/2894
5154/28945523;chr6:890/chr6/29521420/29521624;chr6:920/chr6/30039130/30039476;chr6:1002/chr6/314658
39/31466177;chr6:1147/chr6/33245328/33245537;chr6:1289/chr6/35109073/35109224;chr6:1322/chr6/359922
08/35992409;chr6:1534/chr6/41515897/41516162;chr6:1567/chr6/42072071/42072647;chr6:1630/chr6/432528
33/43252896;chr6:1651/chr6/43970475/43970721;chr6:1665/chr6/44243850/44244010;chr6:1746/chr6/508180
73/50818360;chr6:1788/chr6/54711388/54711661;chr6:1872/chr6/72892268/72892444;chr6:1938/chr6/796203
42/79620474;chr6:1972/chr6/84418789/84419071;chr6:1985/chr6/85483932/85484557;chr6:2032/chr6/888764
77/88876630;chr6:2135/chr6/100051109/100051202;chr6:2142/chr6/100061697/100061988;chr6:2170/chr6/10
0913135/100913285;chr6:2182/chr6/101850539/101850938;chr6:2190/chr6/105389150/105389509;chr6:2232/
chr6/107955829/107956157;chr6:2249/chr6/108438877/108439066;chr6:2263/chr6/108489809/108490066;chr
6:2311/chr6/110797815/110797976;chr6:2384/chr6/117086640/117086902;chr6:2386/chr6/117198221/117198
339;chr6:2396/chr6/117868961/117869524;chr6:2436/chr6/125475590/125475739;chr6:2499/chr6/133562437/
133562674;chr6:2544/chr6/137244729/137245027;chr6:2558/chr6/137809917/137810026;chr6:2842/chr6/1575
57572/157558003;chr6:3358/chr6/168502019/168502200;chr6:3360/chr6/168502504/168502654;chr7:282/chr7/
751765/751986;chr7:405/chr7/1276049/1276436;chr7:547/chr7/1576917/1577229;chr7:562/chr7/1625817/162
6380;chr7:727/chr7/2539832/2540075;chr7:735/chr7/2558425/2558675;chr7:811/chr7/2968281/2968707;chr7:8
73/chr7/3340815/3341286;chr7:874/chr7/3341409/3341561;chr7:1084/chr7/4922707/4922839;chr7:1085/chr7/4
922974/4923327;chr7:1305/chr7/19156133/19156303;chr7:1307/chr7/19156746/19157055;chr7:1376/chr7/232
45561/23245864;chr7:1377/chr7/23287352/23287532;chr7:1422/chr7/25892431/25892608;chr7:1481/chr7/271
27643/27127803;chr7:1486/chr7/27141753/27141936;chr7:1491/chr7/27153135/27153399;chr7:1497/chr7/271
55002/27155372;chr7:1514/chr7/27187502/27187670;chr7:1519/chr7/27195602/27195742;chr7:1520/chr7/271
95893/27196049;chr7:1533/chr7/27204894/27205230;chr7:1536/chr7/27205790/27206082;chr7:1543/chr7/272
12984/27213188;chr7:1552/chr7/27225058/27225249;chr7:1568/chr7/27260167/27260417;chr7:1570/chr7/272
79100/27279237;chr7:1581/chr7/27291201/27291314;chr7:1788/chr7/35293478/35294003;chr7:1858/chr7/374
87454/37487755;chr7:1887/chr7/38670664/38670895;chr7:1908/chr7/39649254/39649443;chr7:1932/chr7/422
67712/42267821;chr7:2143/chr7/49813018/49813809;chr7:2146/chr7/49814751/49815020;chr7:2148/chr7/498
15250/49815543;chr7:2162/chr7/50343490/50343974;chr7:2589/chr7/70596711/70597367;chr7:2591/chr7/705
97872/70597945;chr7:2651/chr7/71801131/71801481;chr7:2696/chr7/72848129/72848426;chr7:3079/chr7/989
71677/98971810;chr7:3100/chr7/99769405/99769557;chr7:3112/chr7/100091181/100091621;chr7:3136/chr7/1
00318111/100318466;chr7:3315/chr7/103630083/103630363;chr7:3399/chr7/112430366/112430590;chr7:3412/
chr7/113727626/113727753;chr7:3421/chr7/115850578/115850693;chr7:3489/chr7/126987840/126988120;chr
7:3500/chr7/127671170/127671450;chr7:3501/chr7/127671658/127672235;chr7:3502/chr7/127672383/127672
785;chr7:3563/chr7/128809071/128809165;chr7:3586/chr7/129421709/129422115;chr7:3587/chr7/129422969/
129423125;chr7:3616/chr7/130418738/130419173;chr7:3763/chr7/139167827/139168114;chr7:3975/chr7/1491
19597/149119681;chr7:3978/chr7/149120132/149120275;chr7:4109/chr7/150655146/150655637;chr7:4146/chr
7/150778541/150778926;chr7:4153/chr7/150811800/150812813;chr7:4303/chr7/152622854/152623254;chr7:4
539/chr7/155579946/155580181;chr7:4544/chr7/155595858/155596136;chr7:4565/chr7/155744234/155744666;
chr7:4691/chr7/156796740/156796978;chr7:4724/chr7/157071962/157072392;chr7:4786/chr7/157406031/157
406394;chr7:4816/chr7/157477489/157477710;chr7:4817/chr7/157477820/157478407;chr7:4818/chr7/1574785
65/157478830;chr7:4820/chr7/157481550/157481759;chr7:5168/chr7/158937479/158937576;chr8:13/chr8/494
181/494828;chr8:33/chr8/638208/638418;chr8:37/chr8/686885/687162;chr8:38/chr8/687290/687513;chr8:39/ch
r8/687642/687837;chr8:40/chr8/688020/688402;chr8:42/chr8/688740/689295;chr8:96/chr8/876824/877094;chr8:
263/chr8/1443908/1444164;chr8:354/chr8/1950230/1950518;chr8:565/chr8/8559652/8559956;chr8:594/chr8/9
764050/9764195;chr8:650/chr8/10587603/10587893;chr8:651/chr8/10588008/10588184;chr8:652/chr8/105882
93/10588608;chr8:653/chr8/10588950/10589092;chr8:656/chr8/10590020/10590289;chr8:728/chr8/11537698/1
1538007;chr8:738/chr8/11559795/11560067;chr8:766/chr8/12809061/12809462;chr8:791/chr8/15397660/1539
7991;chr8:809/chr8/17270826/17271090;chr8:940/chr8/21647110/21647266;chr8:941/chr8/21647456/2164774
6;chr8:1006/chr8/22408759/22409076;chr8:1018/chr8/22446721/22446902;chr8:1033/chr8/22548249/2254840
2;chr8:1101/chr8/23584401/23584581;chr8:1107/chr8/24771125/24771349;chr8:1276/chr8/31497961/3149829
7;chr8:1309/chr8/35092703/35093036;chr8:1349/chr8/37555619/37555753;chr8:1350/chr8/37555912/3755610
4;chr8:1403/chr8/38757652/38757878;chr8:1405/chr8/38831868/38832210;chr8:1408/chr8/38964944/3896532
1;chr8:1695/chr8/53852184/53852565;chr8:1696/chr8/53852914/53853051;chr8:1701/chr8/54163350/5416367
3;chr8:1716/chr8/54789806/54790022;chr8:1720/chr8/54791946/54792184;chr8:1738/chr8/55372068/5537223
6;chr8:1742/chr8/55379457/55379837;chr8:1744/chr8/55382803/55383182;chr8:1752/chr8/56014744/5601503
4;chr8:1788/chr8/57358147/57358464;chr8:1789/chr8/57358662/57358806;chr8:1876/chr8/65282005/6528218
7;chr8:1878/chr8/65282524/65282712;chr8:1893/chr8/65493579/65493806;chr8:1921/chr8/67873422/6787365
5;chr8:1959/chr8/70946915/70947090;chr8:1962/chr8/70981880/70982154;chr8:1971/chr8/72459613/7246029
9;chr8:2051/chr8/80804108/80804323;chr8:2113/chr8/91803720/91804064;chr8:2179/chr8/97157210/9715741
2;chr8:2181/chr8/97157924/97158021;chr8:2236/chr8/99952304/99952532;chr8:2241/chr8/99960498/9996092
1;chr8:2251/chr8/99986044/99986225;chr8:2284/chr8/102505986/102506365;chr8:2423/chr8/120220606/1202
20895;chr8:2717/chr8/139509128/139509450;chr8:2786/chr8/140716022/140716328;chr8:2963/chr8/14242807
4/142428340;chr8:3309/chr8/143545510/143545847;chr8:3322/chr8/143572961/143573153;chr8:3527/chr8/14
4358144/144358360;chr8:3611/chr8/144810477/144810542;chr8:3689/chr8/145052074/145052253;chr8:3701/
chr8/145103842/145104436;chr8:3702/chr8/145105416/145105684;chr8:3703/chr8/145105890/145106676;chr
8:3705/chr8/145106972/145107072;chr8:3759/chr8/145697983/145698667;chr8:3776/chr8/145806272/145806
619;chr9:4/chr9/113513/113549;chr9:47/chr9/976690/976911;chr9:55/chr9/1045628/1045794;chr9:58/chr9/105
0173/1050390;chr9:60/chr9/1051639/1051904;chr9:135/chr9/8857486/8857685;chr9:187/chr9/17134846/17134
993;chr9:193/chr9/17579292/17579402;chr9:207/chr9/19127361/19127549;chr9:239/chr9/23821734/23822017;
chr9:343/chr9/34809764/34810089;chr9:376/chr9/35675648/35675837;chr9:402/chr9/36739634/36739811;chr9:
431/chr9/37002518/37002723;chr9:629/chr9/79635449/79635745;chr9:631/chr9/79637889/79638136;chr9:948/
chr9/94712080/94712294;chr9:1070/chr9/96714034/96714479;chr9:1073/chr9/96715373/96715687;chr9:1080/
chr9/96720886/96721102;chr9:1081/chr9/96721468/96721688;chr9:1083/chr9/96722663/96722862;chr9:1173/
chr9/98981562/98981682;chr9:1188/chr9/99482242/99482542;chr9:1218/chr9/100610906/100611124;chr9:122
5/chr9/100616098/100616270;chr9:1226/chr9/100616469/100617285;chr9:1287/chr9/102590760/102590938;c
hr9:1343/chr9/110250215/110250497;chr9:1374/chr9/112081334/112081563;chr9:1388/chr9/113341848/11334
1926;chr9:1427/chr9/116225742/116226119;chr9:1535/chr9/120507295/120507466;chr9:1682/chr9/126135610/
126135979;chr9:1702/chr9/126773780/126773939;chr9:1705/chr9/126774792/126775213;chr9:1708/chr9/126
777747/126777928;chr9:1715/chr9/126780044/126780284;chr9:1770/chr9/127265610/127265875;chr9:1878/c
hr9/129377352/129377592;chr9:1941/chr9/130461571/130461686;chr9:1956/chr9/130516521/130516802;chr9:
1980/chr9/130689450/130689630;chr9:2117/chr9/132382355/132382634;chr9:2143/chr9/132650763/13265102
5;chr9:2223/chr9/133540232/133540453;chr9:2352/chr9/134430193/134430276;chr9:2443/chr9/135462208/13
5462395;chr9:2530/chr9/136293629/136293841;chr9:2786/chr9/137347895/137348054;chr9:2923/chr9/137967
364/137967472;chr9:3136/chr9/138606249/138606523;chr9:3185/chr9/138674794/138675137;chr9:3255/chr9/
139024521/139024749;chr9:3295/chr9/139093450/139093772;chr9:3297/chr9/139094733/139095014;chr9:349
7/chr9/139715867/139716404;chr9:3504/chr9/139739197/139739297;chr9:3508/chr9/139740650/139740877;c
hr9:3509/chr9/139741288/139741540;chr9:3596/chr9/140033627/140033762;chr9:3751/chr9/140680995/14068
1409;chrX:27/chrX/334496/334693;chrX:132/chrX/933732/933992;chrX:396/chrX/12156923/12156990;chrX:50
3/chrX/21676168/21676438;chrX:504/chrX/21676563/21676656;chrX:537/chrX/25020597/25020805;chrX:666/c
hrX/39864556/39865040;chrX:678/chrX/39949248/39949745;chrX:693/chrX/40004992/40005424;chrX:784/chr
X/47433504/47433757;chrX:810/chrX/48684899/48685724;chrX:1246/chrX/99663657/99663802;chrX:1339/chr
X/108868335/108868474;chrX:1560/chrX/133684807/133685051;chrX:1756/chrX/150863411/150863573;chrX:
1761/chrX/151142882/151143101;chrY:18/chrY/7141863/7142122

TABLE 2
chr1/1362151/1362490/chr1:910;chr1/1369570/1370068/chr1:925;chr1/1475163/1475494/chr1:1018;chr1/1564
455/1565143/chr1:1116;chr1/1564455/1565143/chr1:1116;chr1/1564455/1565143/chr1:1116;chr1/1830655/183
0795/chr1:1274;chr1/2080122/2080394/chr1:1594;chr1/2231795/2232310/chr1:1731;chr1/2705890/2705935/ch
r1:2426;chr1/3454818/3454991/chr1:4330;chr1/3567271/3567590/chr1:4499;chr1/3567271/3567590/chr1:4499;
chr1/3595087/3595205/chr1:4556;chr1/6569441/6569670/chr1:8311;chr1/10948524/10948737/chr1:11907;chr1/
13910785/13910996/chr1:14264;chr1/17446428/17446468/chr1:16499;chr1/18958207/18958318/chr1:18173;c
hr1/18958207/18958318/chr1:18173;chr1/19600483/19600729/chr1:18701;chr1/21619976/21620270/chr1:1997
2;chr1/21754735/21754862/chr1:20054;chr1/22150069/22150464/chr1:20290;chr1/23279738/23280152/chr1:2
0982;chr1/24229453/24229473/chr1:21457;chr1/24514556/24514763/chr1:21620;chr1/26097990/26098102/chr
1:22540;chr1/27159834/27160220/chr1:23197;chr1/27687297/27687463/chr1:23491;chr1/27687297/27687463/
chr1:23491;chr1/32237536/32238659/chr1:26783;chr1/32716772/32716888/chr1:27028;chr1/34642382/346425
55/chr1:28244;chr1/36028848/36029038/chr1:29452;chr1/36787355/36787413/chr1:29780;chr1/40236970/402
37021/chr1:33168;chr1/40426119/40426244/chr1:33307;chr1/43533791/43534323/chr1:35016;chr1/44031248/
44031805/chr1:35215;chr1/45275896/45275995/chr1:35807;chr1/46632274/46632996/chr1:36254;chr1/540408
43/54041029/chr1:40129;chr1/54941110/54941446/chr1:40507;chr1/63787249/63787461/chr1:44946;chr1/657
31468/65731915/chr1:45729;chr1/67773922/67774123/chr1:46521;chr1/87597973/87598463/chr1:57617;chr1/
91192577/91192791/chr1:59252;chr1/91192577/91192791/chr1:59252;chr1/91306962/91307009/chr1:59355;c
hr1/110612669/110612826/chr1:70099;chr1/110612669/110612826/chr1:70099;chr1/113286995/113287154/ch
r1:71764;chr1/119526993/119527544/chr1:74779;chr1/119526993/119527544/chr1:74779;chr1/119527718/119
527825/chr1:74780;chr1/119530143/119530243/chr1:74786;chr1/119532021/119532194/chr1:74790;chr1/1195
32773/119533069/chr1:74792;chr1/119550936/119551175/chr1:74822;chr1/120906033/120906339/chr1:75227;
chr1/151810460/151810772/chr1:78216;chr1/151810460/151810772/chr1:78216;chr1/151810886/151811540/
chr1:78217;chr1/151810886/151811540/chr1:78217;chr1/154475268/154475330/chr1:79838;chr1/156862708/1
56863308/chr1:81300;chr1/156863415/156863709/chr1:81301;chr1/156863415/156863709/chr1:81301;chr1/16
1038973/161039694/chr1:84750;chr1/180202382/180203142/chr1:94606;chr1/180202382/180203142/chr1:946
06;chr1/180204498/180204795/chr1:94610;chr1/180204498/180204795/chr1:94610;chr1/182025825/18202595
9/chr1:95677;chr1/182921856/182921912/chr1:96009;chr1/182922062/182922174/chr1:96010;chr1/197887676/
197887784/chr1:104446;chr1/200842542/200843206/chr1:105831;chr1/201708887/201709532/chr1:106285;c
hr1/202130663/202130966/chr1:106485;chr1/202168501/202168757/chr1:106517;chr1/203044923/203045258/
chr1:106992;chr1/203044923/203045258/chr1:106992;chr1/203044923/203045258/chr1:106992;chr1/2046534
56/204653670/chr1:107956;chr1/209404922/209405233/chr1:110626;chr1/212688031/212688332/chr1:112342;
chr1/212688031/212688332/chr1:112342;chr1/217310297/217310421/chr1:114639;chr1/221049969/22105011
5/chr1:116503;chr1/225654936/225655183/chr1:118864;chr1/225655594/225655776/chr1:118866;chr1/226613
283/226613688/chr1:119294;chr1/229542753/229543124/chr1:120842;chr1/229542753/229543124/chr1:12084
2;chr1/231296446/231296828/chr1:122207;chr1/231296446/231296828/chr1:122207;chr1/235586791/2355869
01/chr1:124682;chr10/1778903/1779261/chr10:1756;chr10/7449577/7449896/chr10:7545;chr10/8075813/8075
974/chr10:7871;chr10/8076091/8076417/chr10:7872;chr10/8076091/8076417/chr10:7872;chr10/8090398/8090
524/chr10:7905;chr10/11207431/11207689/chr10:10698;chr10/11207431/11207689/chr10:10698;chr10/112074
31/11207689/chr10:10698;chr10/22542297/22542380/chr10:18840;chr10/23482254/23482428/chr10:19329;chr
10/26500995/26501046/chr10:21693;chr10/35928211/35928712/chr10:26395;chr10/35928896/35928953/chr10:
26396;chr10/43429116/43429442/chr10:29126;chr10/49892740/49892954/chr10:32627;chr10/77156080/7715
6191/chr10:49397;chr10/81742979/81743380/chr10:52751;chr10/88296200/88296314/chr10:58339;chr10/8829
6422/88296712/chr10:58340;chr10/88296422/88296712/chr10:58340;chr10/88438958/88439043/chr10:58403;
chr10/94450625/94451827/chr10:61217;chr10/94450625/94451827/chr10:61217;chr10/94451928/94451992/ch
r10:61218;chr10/94825896/94825954/chr10:61360;chr10/94835205/94835386/chr10:61364;chr10/102892565/1
02892633/chr10:65984;chr10/103534322/103534488/chr10:66305;chr10/103535661/103536004/chr10:66310;c
hr10/105344355/105344542/chr10:67145;chr10/110225994/110226376/chr10:72252;chr10/111216604/111216
677/chr10:73103;chr10/118891689/118891759/chr10:78180;chr10/118900240/118900277/chr10:78195;chr10/1
21300969/121301307/chr10:80075;chr10/121300969/121301307/chr10:80075;chr10/123922703/123922731/ch
r10:81707;chr10/123923178/123923629/chr10:81709;chr10/123923178/123923629/chr10:81709;chr10/123923
771/123924254/chr10:81710;chr10/125651285/125651525/chr10:83024;chr10/126135809/126136050/chr10:83
283;chr10/126223218/126223262/chr10:83347;chr10/126840822/126841011/chr10:83631;chr10/130508374/13
0508569/chr10:86629;chr10/134000464/134001032/chr10:92085;chr10/134000464/134001032/chr10:92085;ch
r10/134000464/134001032/chr10:92085;chr10/134000464/134001032/chr10:92085;chr10/134357813/1343581
74/chr10:92595;chr11/415261/415621/chr11:259;chr11/626576/627510/chr11:428;chr11/626576/627510/chr11:
428;chr11/626576/627510/chr11:428;chr11/636988/637191/chr11:435;chr11/637329/637874/chr11:436;chr11/6
37329/637874/chr11:436;chr11/637329/637874/chr11:436;chr11/639470/639835/chr11:437;chr11/639954/6402
72/chr11:438;chr11/720490/720942/chr11:482;chr11/720490/720942/chr11:482;chr11/720490/720942/chr11:48
2;chr11/725615/725807/chr11:489;chr11/725615/725807/chr11:489;chr11/1779712/1779972/chr11:2023;chr11/
1873925/1874469/chr11:2166;chr11/1910395/1910519/chr11:2210;chr11/1910627/1911057/chr11:2211;chr11/
2883553/2884171/chr11:3216;chr11/2891153/2891178/chr11:3224;chr11/3150308/3150439/chr11:3466;chr11/
9112508/9112702/chr11:7541;chr11/14926953/14926984/chr11:9705;chr11/14927196/14927368/chr11:9707;c
hr11/16632058/16632416/chr11:10457;chr11/19264030/19264095/chr11:11957;chr11/20618888/20619024/chr
11:12594;chr11/22454316/22454382/chr11:14158;chr11/31839928/31840018/chr11:20769;chr11/31840660/31
841216/chr11:20771;chr11/47416141/47416634/chr11:32772;chr11/60718476/60718969/chr11:40818;chr11/61
322897/61322944/chr11:41211;chr11/61723329/61723400/chr11:41477;chr11/62693938/62694183/chr11:4222
7;chr11/63974368/63974719/chr11:43046;chr11/64108022/64108251/chr11:43147;chr11/64108022/64108251/
chr11:43147;chr11/65313877/65314032/chr11:44026;chr11/65313877/65314032/chr11:44026;chr11/65408358/
65408898/chr11:44094;chr11/65661460/65661570/chr11:44327;chr11/66624890/66624939/chr11:44938;chr11/
67164679/67164799/chr11:45215;chr11/67171014/67171199/chr11:45223;chr11/67176901/67177120/chr11:45
235;chr11/67176901/67177120/chr11:45235;chr11/67176901/67177120/chr11:45235;chr11/67414130/6741447
9/chr11:45465;chr11/68847931/68848056/chr11:46495;chr11/69919937/69920113/chr11:47246;chr11/7025698
6/70257176/chr11:47406;chr11/70278205/70278278/chr11:47424;chr11/70508152/70508658/chr11:47557;chr1
1/76032790/76033382/chr11:50007;chr11/94473595/94473987/chr11:59990;chr11/94770846/94771101/chr11:
60094;chr11/111093651/111093897/chr11:69530;chr11/111783442/111783600/chr11:69878;chr11/116371471/
116371540/chr11:73717;chr11/124629401/124629621/chr11:80450;chr11/124735322/124735411/chr11:80531;
chr11/125036324/125036457/chr11:80676;chr11/129242392/129242675/chr11:85119;chr11/131780380/13178
0533/chr11:87139;chr11/131781045/131781370/chr11:87141;chr11/132953069/132953147/chr11:88839;chr11/
133994774/133994914/chr11:90512;chr12/312736/313096/chr12:270;chr12/662788/662960/chr12:455;chr12/1
756220/1756344/chr12:896;chr12/2393217/2393554/chr12:1364;chr12/2800420/2800664/chr12:1587;chr12/28
00420/2800664/chr12:1587;chr12/2903453/2903531/chr12:1688;chr12/3309931/3310301/chr12:1891;chr12/61
84504/6184741/chr12:3862;chr12/6427770/6427847/chr12:4070;chr12/7060367/7061427/chr12:4519;chr12/70
61550/7061715/chr12:4520;chr12/7067956/7068210/chr12:4531;chr12/8025364/8025803/chr12:5141;chr12/80
25364/8025803/chr12:5141;chr12/8025364/8025803/chr12:5141;chr12/22487006/22487306/chr12:13815;chr12/
25055380/25055872/chr12:15769;chr12/25055985/25056160/chr12:15770;chr12/30322960/30323020/chr12:1
8141;chr12/34260676/34261176/chr12:20077;chr12/51717537/51717922/chr12:27049;chr12/52644089/526443
80/chr12:27804;chr12/52652267/52652341/chr12:27808;chr12/52652454/52652537/chr12:27809;chr12/540885
35/54088968/chr12:28935;chr12/54338742/54338951/chr12:29164;chr12/54409211/54409267/chr12:29251;chr
12/54423548/54423710/chr12:29267;chr12/54424627/54424963/chr12:29270;chr12/54424627/54424963/chr12:
29270;chr12/54428282/54428663/chr12:29273;chr12/54428282/54428663/chr12:29273;chr12/54811864/5481
2113/chr12:29501;chr12/57618538/57618942/chr12:31789;chr12/57618538/57618942/chr12:31789;chr12/8110
1788/81101900/chr12:46014;chr12/81102034/81102200/chr12:46015;chr12/85672277/85672402/chr12:49063;
chr12/111847251/111847387/chr12:64298;chr12/114834134/114834256/chr12:66791;chr12/114846822/11484
6948/chr12:66816;chr12/114847527/114847629/chr12:66817;chr12/115102537/115102918/chr12:67067;chr12/
115103155/115104295/chr12:67069;chr12/115103155/115104295/chr12:67069;chr12/115103155/115104295/c
hr12:67069;chr12/115104848/115105484/chr12:67071;chr12/115104848/115105484/chr12:67071;chr12/11510
9640/115110037/chr12:67076;chr12/115109640/115110037/chr12:67076;chr12/115123149/115123483/chr12:6
7087;chr12/115123149/115123483/chr12:67087;chr12/115124686/115125224/chr12:67091;chr12/115173215/1
15173938/chr12:67149;chr12/120032389/120032522/chr12:70924;chr12/121530597/121530897/chr12:71725;c
hr12/122016572/122016668/chr12:71954;chr12/122227752/122228315/chr12:72119;chr12/122230877/122231
185/chr12:72126;chr12/122442162/122442462/chr12:72253;chr12/122669142/122669439/chr12:72395;chr12/1
23518598/123518703/chr12:72774;chr12/124781191/124781369/chr12:73584;chr12/124941722/124941910/ch
r12:73651;chr12/125039218/125039478/chr12:73704;chr12/130388702/130388773/chr12:81010;chr12/130388
702/130388773/chr12:81010;chr12/130647935/130648847/chr12:81367;chr12/130647935/130648847/chr12:81
367;chr12/130647935/130648847/chr12:81367;chr12/130647935/130648847/chr12:81367;chr12/130647935/13
0648847/chr12:81367;chr12/130647935/130648847/chr12:81367;chr12/130647935/130648847/chr12:81367;ch
r12/130648983/130649264/chr12:81368;chr12/132337575/132337658/chr12:83914;chr12/132381543/1323817
82/chr12:83961;chr12/132906202/132906398/chr12:84438;chr12/133021467/133021706/chr12:84684;chr13/21
520154/21520551/chr13:1827;chr13/23500738/23501465/chr13:2889;chr13/24839234/24839454/chr13:3848;c
hr13/25506171/25506339/chr13:4241;chr13/26586648/26586932/chr13:4985;chr13/28370464/28370525/chr13:
6364;chr13/28493315/28493426/chr13:6530;chr13/28501983/28502043/chr13:6545;chr13/47012284/47012436/
chr13:17668;chr13/50701411/50701867/chr13:19654;chr13/50706829/50707124/chr13:19660;chr13/50707538/
50707743/chr13:19662;chr13/50707538/50707743/chr13:19662;chr13/95354896/95355135/chr13:57624;chr13/
95354896/95355135/chr13:57624;chr13/96294429/96294637/chr13:58509;chr13/100310204/100311032/chr13:
60894;chr13/100547539/100548456/chr13:61035;chr13/100547539/100548456/chr13:61035;chr13/100547539/
100548456/chr13:61035;chr13/100620241/100620554/chr13:61109;chr13/100622079/100622093/chr13:61112;
chr13/100641680/100641899/chr13:61131;chr13/100643761/100643791/chr13:61137;chr13/100649622/10064
9799/chr13:61140;chr13/100649911/100650222/chr13:61141;chr13/102568810/102568923/chr13:62023;chr13/
109792746/109793137/chr13:69578;chr13/109792746/109793137/chr13:69578;chr13/109793269/109793379/c
hr13:69579;chr13/110950743/110950812/chr13:70252;chr13/111719002/111719171/chr13:70691;chr13/11254
8012/112548105/chr13:71605;chr13/112710293/112710427/chr13:71891;chr13/112710562/112710853/chr13:7
1892;chr13/112711361/112711613/chr13:71894;chr13/112717195/112717377/chr13:71911;chr13/112721711/1
12721794/chr13:71921;chr13/112721942/112722094/chr13:71922;chr13/112726293/112726465/chr13:71929;c
hr13/112758598/112759112/chr13:71963;chr13/112758598/112759112/chr13:71963;chr13/112759905/112760
087/chr13:71966;chr13/114932211/114932339/chr13:74756;chr14/21091033/21091491/chr14:1037;chr14/2109
2115/21092183/chr14:1038;chr14/24601611/24601869/chr14:4094;chr14/24641229/24641404/chr14:4110;chr1
4/24779828/24780851/chr14:4166;chr14/29254416/29254621/chr14:8618;chr14/29254416/29254621/chr14:86
18;chr14/34269830/34269915/chr14:11353;chr14/37053189/37053429/chr14:12842;chr14/37053189/37053429/
chr14:12842;chr14/37131997/37132554/chr14:12902;chr14/37135513/37135987/chr14:12907;chr14/37136098/
37136346/chr14:12908;chr14/37136098/37136346/chr14:12908;chr14/38060856/38061016/chr14:13797;chr14/
38067835/38067988/chr14:13810;chr14/38725360/38725529/chr14:14475;chr14/45381268/45381711/chr14:2
0066;chr14/52734955/52735069/chr14:25597;chr14/52735420/52735567/chr14:25599;chr14/54686034/546867
07/chr14:26279;chr14/54686034/54686707/chr14:26279;chr14/57261408/57261631/chr14:27756;chr14/572614
08/57261631/chr14:27756;chr14/57261756/57262025/chr14:27757;chr14/57264609/57264918/chr14:27762;chr
14/57264609/57264918/chr14:27762;chr14/57265460/57265539/chr14:27765;chr14/57270914/57271214/chr14:
27770;chr14/57270914/57271214/chr14:27770;chr14/60982558/60982691/chr14:30223;chr14/61103912/6110
4315/chr14:30325;chr14/61104451/61104661/chr14:30326;chr14/73180974/73181293/chr14:35585;chr14/7371
2213/73712414/chr14:35748;chr14/74101076/74101141/chr14:35888;chr14/74981396/74981616/chr14:36307;
chr14/77736867/77737161/chr14:38034;chr14/91287359/91287583/chr14:48657;chr14/93022169/93022311/ch
r14:49730;chr14/95235401/95235539/chr14:51665;chr14/95237662/95237983/chr14:51668;chr14/95826716/95
826939/chr14:52052;chr14/97093479/97093525/chr14:53073;chr14/101924168/101924293/chr14:58757;chr14/
101924419/101924564/chr14:58758;chr14/103389888/103390183/chr14:59703;chr14/103568956/103568982/c
hr14:59807;chr14/103740306/103740714/chr14:59940;chr14/103745833/103746148/chr14:59942;chr14/10374
5833/103746148/chr14:59942;chr14/105527220/105527664/chr14:62194;chr14/105527220/105527664/chr14:6
2194;chr14/105888382/105888765/chr14:62461;chr14/105888382/105888765/chr14:62461;chr14/105944677/1
05944743/chr14:62497;chr15/20499795/20500710/chr15:242;chr15/26107480/26107782/chr15:3677;chr15/271
13169/27113239/chr15:4764;chr15/29033895/29033947/chr15:6658;chr15/29034556/29034805/chr15:6660;chr
15/30517493/30517588/chr15:7682;chr15/31727074/31727382/chr15:8312;chr15/32605407/32605952/chr15:9
254;chr15/37172556/37172678/chr15:11520;chr15/37389383/37389685/chr15:11604;chr15/37390188/3739028
8/chr15:11607;chr15/41217728/41218050/chr15:13757;chr15/41794708/41795295/chr15:13969;chr15/4179470
8/41795295/chr15:13969;chr15/41794708/41795295/chr15:13969;chr15/41803519/41804754/chr15:13976;chr1
5/41803519/41804754/chr15:13976;chr15/53093094/53093493/chr15:18461;chr15/53093094/53093493/chr15:
18461;chr15/53097869/53097985/chr15:18470;chr15/53098107/53098456/chr15:18471;chr15/60297884/60297
932/chr15:22825;chr15/65116033/65116193/chr15:25762;chr15/65688922/65689360/chr15:26026;chr15/68851
465/68851534/chr15:27484;chr15/74429663/74429805/chr15:31086;chr15/76630511/76630659/chr15:32290;c
hr15/76634720/76635202/chr15:32302;chr15/76634720/76635202/chr15:32302;chr15/77288814/77289046/chr
15:32444;chr15/79123038/79123313/chr15:33594;chr15/79381804/79381850/chr15:33715;chr15/88800985/88
801136/chr15:40067;chr15/89920854/89921235/chr15:41166;chr15/89920854/89921235/chr15:41166;chr15/89
920854/89921235/chr15:41166;chr15/89922271/89922327/chr15:41167;chr15/89949311/89949671/chr15:4118
4;chr15/89949311/89949671/chr15:41184;chr15/89951464/89951622/chr15:41192;chr15/89951965/89952527/
chr15:41194;chr15/90774299/90774778/chr15:41596;chr15/93592143/93592359/chr15:44598;chr15/93616825/
93617134/chr15:44610;chr15/96884272/96885023/chr15:45832;chr15/96902737/96902846/chr15:45874;chr15/
96908794/96910753/chr15:45890;chr15/96911457/96912424/chr15:45892;chr15/96912891/96912960/chr15:45
894;chr15/96951691/96952169/chr15:45910;chr15/101513277/101513644/chr15:49270;chr15/101513277/1015
13644/chr15:49270;chr15/101513277/101513644/chr15:49270;chr15/101991827/101991942/chr15:49533;chr1
6/375242/375429/chr16:220;chr16/1203996/1204053/chr16:1429;chr16/2557412/2557497/chr16:2818;chr16/11
326816/11327384/chr16:12066;chr16/14397377/14397655/chr16:14618;chr16/14397778/14397915/chr16:1461
9;chr16/23706241/23706536/chr16:21325;chr16/29160128/29160605/chr16:27230;chr16/29675696/29676499/
chr16:27687;chr16/29675696/29676499/chr16:27687;chr16/30195448/30195758/chr16:28073;chr16/30484943/
30485169/chr16:28192;chr16/31075642/31075996/chr16:28562;chr16/33852977/33853622/chr16:30088;chr16/
49314761/49314896/chr16:32667;chr16/49872457/49872979/chr16:33144;chr16/49872457/49872979/chr16:33
144;chr16/50307937/50308652/chr16:33364;chr16/50715134/50715410/chr16:33521;chr16/51168302/5116843
3/chr16:33899;chr16/54611546/54611830/chr16:36658;chr16/54966651/54966709/chr16:36806;chr16/5766348
0/57663912/chr16:38765;chr16/65156845/65156992/chr16:46615;chr16/66462124/66462423/chr16:47822;chr1
6/66612664/66613368/chr16:47903;chr16/66612664/66613368/chr16:47903;chr16/67199629/67199743/chr16:
48158;chr16/67199629/67199743/chr16:48158;chr16/67199853/67199986/chr16:48159;chr16/67199853/67199
986/chr16:48159;chr16/67428474/67428777/chr16:48271;chr16/67428883/67429159/chr16:48272;chr16/68390
077/68390872/chr16:48693;chr16/70771490/70771858/chr16:49783;chr16/72821080/72821436/chr16:51214;c
hr16/73099971/73100184/chr16:51363;chr16/73099971/73100184/chr16:51363;chr16/85436723/85436872/chr
16:60934;chr16/85482199/85482903/chr16:60969;chr16/86544675/86544950/chr16:62320;chr16/86544675/86
544950/chr16:62320;chr16/86544675/86544950/chr16:62320;chr16/86612933/86613031/chr16:62392;chr16/88
758409/88758569/chr16:65381;chr16/88766739/88767518/chr16:65392;chr16/88769383/88770591/chr16:6539
7;chr16/88812231/88812447/chr16:65431;chr16/89044989/89045022/chr16:65834;chr16/89789006/89789812/
chr16:66818;chr16/90075489/90075761/chr16:67015;chr17/943291/943384/chr17:999;chr17/1160026/1160310/
chr17:1303;chr17/1173973/1174384/chr17:1314;chr17/1173973/1174384/chr17:1314;chr17/1959355/1960091/
chr17:1996;chr17/1960540/1961482/chr17:1999;chr17/1960540/1961482/chr17:1999;chr17/1960540/1961482/
chr17:1999;chr17/2839062/2839216/chr17:2615;chr17/3438921/3439100/chr17:3270;chr17/3814690/3814963/
chr17:3589;chr17/3819295/3819476/chr17:3594;chr17/4803470/4803791/chr17:4290;chr17/4804169/4804321/
chr17:4291;chr17/5487112/5487267/chr17:4715;chr17/5783170/5783321/chr17:4912;chr17/5800829/5801072/
chr17:4923;chr17/6945866/6946175/chr17:6279;chr17/7077418/7077906/chr17:6388;chr17/7240012/7240105/
chr17:6501;chr17/7341478/7341615/chr17:6617;chr17/7350281/7350422/chr17:6628;chr17/7942502/7942893/
chr17:7021;chr17/7975744/7975931/chr17:7053;chr17/12876973/12877815/chr17:11258;chr17/12876973/1287
7815/chr17:11258;chr17/12876973/12877815/chr17:11258;chr17/15820655/15821098/chr17:12841;chr17/1729
5317/17295718/chr17:13762;chr17/27943684/27944078/chr17:18153;chr17/32964499/32965031/chr17:21612;
chr17/33672620/33672971/chr17:22341;chr17/36104338/36105581/chr17:23923;chr17/36104338/36105581/ch
r17:23923;chr17/36717904/36717999/chr17:24289;chr17/37307985/37308082/chr17:24659;chr17/38334264/38
334751/chr17:25207;chr17/40440835/40441569/chr17:26391;chr17/40440835/40441569/chr17:26391;chr17/40
821935/40822927/chr17:26581;chr17/40821935/40822927/chr17:26581;chr17/40825733/40825840/chr17:2658
6;chr17/40825733/40825840/chr17:26586;chr17/43037172/43037499/chr17:27980;chr17/43198257/43198771/
chr17:28085;chr17/43198257/43198771/chr17:28085;chr17/43198257/43198771/chr17:28085;chr17/43221565/
43221593/chr17:28100;chr17/44846903/44847450/chr17:29242;chr17/46646010/46646043/chr17:30464;chr17/
46647721/46647907/chr17:30465;chr17/46663647/46663942/chr17:30478;chr17/46669643/46669915/chr17:30
489;chr17/46670024/46670107/chr17:30490;chr17/46915331/46915460/chr17:30721;chr17/47302293/4730235
0/chr17:30992;chr17/48042078/48043118/chr17:31431;chr17/48042078/48043118/chr17:31431;chr17/4854611
1/48546512/chr17:31818;chr17/48546111/48546512/chr17:31818;chr17/48546619/48547082/chr17:31819;chr1
7/55123208/55123987/chr17:37192;chr17/55123208/55123987/chr17:37192;chr17/55951927/55952247/chr17:
37573;chr17/56410216/56410519/chr17:37988;chr17/56564865/56565663/chr17:38121;chr17/56564865/56565
663/chr17:38121;chr17/56564865/56565663/chr17:38121;chr17/56564865/56565663/chr17:38121;chr17/56564
865/56565663/chr17:38121;chr17/59481673/59482955/chr17:39251;chr17/59481673/59482955/chr17:39251;c
hr17/59481673/59482955/chr17:39251;chr17/59485485/59485688/chr17:39254;chr17/59529124/59529366/chr
17:39274;chr17/59532563/59532880/chr17:39282;chr17/61512340/61512792/chr17:40085;chr17/61778275/61
778957/chr17:40321;chr17/61778275/61778957/chr17:40321;chr17/62097845/62098021/chr17:40630;chr17/73
073809/73073887/chr17:48787;chr17/73749586/73750133/chr17:49272;chr17/73749586/73750133/chr17:4927
2;chr17/73749586/73750133/chr17:49272;chr17/73749586/73750133/chr17:49272;chr17/74070524/74070599/
chr17:49518;chr17/74117922/74118119/chr17:49546;chr17/74118230/74118311/chr17:49547;chr17/74136412/
74136500/chr17:49564;chr17/74994406/74994570/chr17:50255;chr17/75370153/75370205/chr17:50533;chr17/
75372356/75372767/chr17:50539;chr17/76127034/76127922/chr17:51707;chr17/76127034/76127922/chr17:51
707;chr17/77751046/77751088/chr17:53744;chr17/77789377/77789840/chr17:53782;chr17/77789377/7778984
0/chr17:53782;chr17/77789377/77789840/chr17:53782;chr17/77814197/77814304/chr17:53802;chr17/7791673
2/77916954/chr17:53874;chr17/78992444/78992563/chr17:54585;chr17/79315186/79315908/chr17:54860;chr1
7/79315186/79315908/chr17:54860;chr17/79315186/79315908/chr17:54860;chr17/79316375/79316989/chr17:
54862;chr17/79450367/79450424/chr17:55083;chr17/79620489/79620665/chr17:55198;chr17/80057302/80057
545/chr17:55526;chr17/80943940/80943999/chr17:56277;chr17/81147353/81147508/chr17:56508;chr18/10032
400/10032969/chr18:6343;chr18/10032400/10032969/chr18:6343;chr18/12254751/12254869/chr18:8797;chr18/
12911287/12911613/chr18:9184;chr18/12911287/12911613/chr18:9184;chr18/12911287/12911613/chr18:918
4;chr18/12911287/12911613/chr18:9184;chr18/13136145/13136425/chr18:9249;chr18/14748156/14748450/chr
18:10861;chr18/14748156/14748450/chr18:10861;chr18/14748156/14748450/chr18:10861;chr18/14999432/14
999523/chr18:10974;chr18/31803614/31803759/chr18:19568;chr18/45058208/45058295/chr18:31262;chr18/46
361057/46361418/chr18:31851;chr18/55019905/55019959/chr18:37805;chr18/55094803/55095319/chr18:3790
7;chr18/55095482/55096174/chr18:37908;chr18/55095482/55096174/chr18:37908;chr18/55095482/55096174/
chr18:37908;chr18/55103169/55103433/chr18:37928;chr18/55103602/55103738/chr18:37929;chr18/55107865/
55107886/chr18:37942;chr18/56935445/56935577/chr18:38909;chr18/70536114/70536532/chr18:51967;chr18/
70536114/70536532/chr18:51967;chr18/76733786/76733857/chr18:60480;chr18/77159349/77159419/chr18:60
720;chr18/77397574/77397817/chr18:61213;chr18/77397574/77397817/chr18:61213;chr18/77547953/7754815
5/chr18:61351;chr18/77547953/77548155/chr18:61351;chr19/681322/681892/chr19:695;chr19/681322/681892/
chr19:695;chr19/695454/695563/chr19:714;chr19/695761/695878/chr19:715;chr19/709772/709937/chr19:737;c
hr19/863768/863877/chr19:973;chr19/947650/947711/chr19:1062;chr19/1068360/1068762/chr19:1215;chr19/1
068360/1068762/chr19:1215;chr19/1088234/1088633/chr19:1250;chr19/1132860/1133000/chr19:1294;chr19/1
214133/1214309/chr19:1372;chr19/1363421/1363619/chr19:1563;chr19/1467329/1467398/chr19:1652;chr19/1
855611/1855711/chr19:2248;chr19/1966993/1967352/chr19:2356;chr19/2239836/2240268/chr19:2551;chr19/3
136339/3136709/chr19:3582;chr19/3136339/3136709/chr19:3582;chr19/3178296/3180211/chr19:3636;chr19/3
178296/3180211/chr19:3636;chr19/3178296/3180211/chr19:3636;chr19/3178296/3180211/chr19:3636;chr19/3
178296/3180211/chr19:3636;chr19/3178296/3180211/chr19:3636;chr19/3180319/3180411/chr19:3637;chr19/3
605082/3605239/chr19:4230;chr19/3670917/3671007/chr19:4276;chr19/3671185/3671681/chr19:4277;chr19/3
671783/3672096/chr19:4278;chr19/3950718/3950935/chr19:4734;chr19/4108747/4109297/chr19:4859;chr19/4
543388/4543561/chr19:5308;chr19/6234224/6234309/chr19:7120;chr19/6753333/6753626/chr19:7766;chr19/6
772369/6773097/chr19:7783;chr19/8657676/8657781/chr19:9795;chr19/9473616/9473855/chr19:10645;chr19/
10655643/10655756/chr19:11795;chr19/11253451/11253533/chr19:12176;chr19/11532066/11532221/chr19:12
408;chr19/11785145/11785188/chr19:12612;chr19/12624211/12624564/chr19:12974;chr19/12624211/1262456
4/chr19:12974;chr19/13123020/13123217/chr19:13419;chr19/13215040/13215386/chr19:13579;chr19/1361690
6/13617266/chr19:14187;chr19/14097426/14097579/chr19:14773;chr19/16197073/16197590/chr19:17606;chr1
9/17439581/17439630/chr19:18517;chr19/17889291/17889570/chr19:19186;chr19/18113363/18113567/chr19:
19504;chr19/18402223/18402286/chr19:19820;chr19/33794745/33794972/chr19:29777;chr19/34990900/34991
365/chr19:31007;chr19/34991487/34991990/chr19:31008;chr19/38877229/38877456/chr19:33911;chr19/39228
070/39228317/chr19:34292;chr19/39738337/39738638/chr19:34704;chr19/39738337/39738638/chr19:34704;c
hr19/39738337/39738638/chr19:34704;chr19/39756002/39756142/chr19:34725;chr19/40732229/40732400/chr
19:35463;chr19/40780515/40780792/chr19:35478;chr19/41116456/41116523/chr19:35758;chr19/42828083/42
828110/chr19:37792;chr19/45608025/45608140/chr19:39884;chr19/45662231/45662413/chr19:39923;chr19/45
888797/45889400/chr19:40109;chr19/45899562/45900313/chr19:40118;chr19/45899562/45900313/chr19:4011
8;chr19/46318345/46318671/chr19:40434;chr19/46319243/46319378/chr19:40437;chr19/46997389/46997650/
chr19:41138;chr19/46997774/46998109/chr19:41139;chr19/47152539/47152632/chr19:41309;chr19/47152539/
47152632/chr19:41309;chr19/48048788/48049038/chr19:42058;chr19/48919173/48919323/chr19:42870;chr19/
48919173/48919323/chr19:42870;chr19/48945908/48947360/chr19:42883;chr19/48945908/48947360/chr19:42
883;chr19/48945908/48947360/chr19:42883;chr19/48945908/48947360/chr19:42883;chr19/48945908/4894736
0/chr19:42883;chr19/48945908/48947360/chr19:42883;chr19/48945908/48947360/chr19:42883;chr19/4894590
8/48947360/chr19:42883;chr19/48945908/48947360/chr19:42883;chr19/49263884/49263985/chr19:43144;chr1
9/50058079/50058328/chr19:43919;chr19/51169659/51169792/chr19:45121;chr19/51171368/51171435/chr19:
45124;chr19/52104430/52104969/chr19:46656;chr19/52206690/52206801/chr19:46722;chr19/53966544/53966
573/chr19:47703;chr19/54896046/54896273/chr19:48913;chr19/54976482/54976518/chr19:49026;chr19/55593
207/55593962/chr19:50024;chr19/55593207/55593962/chr19:50024;chr19/55593207/55593962/chr19:50024;c
hr19/55597822/55597916/chr19:50027;chr19/55598140/55598186/chr19:50028;chr19/55765849/55766155/chr
19:50263;chr19/55766832/55767093/chr19:50266;chr19/55896036/55896379/chr19:50417;chr19/58220099/58
220571/chr19:52471;chr19/58867686/58868291/chr19:52906;chr19/58867686/58868291/chr19:52906;chr19/58
867686/58868291/chr19:52906;chr19/58867686/58868291/chr19:52906;chr19/58867686/58868291/chr19:5290
6;chr19/58867686/58868291/chr19:52906;chr19/58908002/58908213/chr19:52949;chr19/58908002/58908213/
chr19:52949;chr2/636653/636940/chr2:745;chr2/636653/636940/chr2:745;chr2/3751088/3751409/chr2:5343;ch
r2/3751088/3751409/chr2:5343;chr2/3751088/3751409/chr2:5343;chr2/8715543/8715853/chr2:10052;chr2/881
6842/8817368/chr2:10123;chr2/10231398/10231780/chr2:10797;chr2/19560698/19561183/chr2:15824;chr2/20
869770/20869861/chr2:16535;chr2/25433406/25433482/chr2:20009;chr2/25499503/25500398/chr2:20054;chr2/
27529715/27529935/chr2:21196;chr2/31805816/31805890/chr2:23778;chr2/31805816/31805890/chr2:23778;c
hr2/38302146/38302381/chr2:28002;chr2/43397997/43398270/chr2:30608;chr2/43397997/43398270/chr2:3060
8;chr2/43451232/43452663/chr2:30651;chr2/45168079/45168250/chr2:31540;chr2/45170446/45170708/chr2:3
1546;chr2/45170446/45170708/chr2:31546;chr2/45227644/45227824/chr2:31626;chr2/45228580/45228639/chr
2:31629;chr2/47206751/47206964/chr2:32641;chr2/47799119/47799129/chr2:32907;chr2/63274639/63275033/
chr2:41614;chr2/63274639/63275033/chr2:41614;chr2/63274639/63275033/chr2:41614;chr2/63278830/632794
22/chr2:41621;chr2/63279839/63280239/chr2:41623;chr2/63279839/63280239/chr2:41623;chr2/63285949/632
86163/chr2:41641;chr2/63286304/63286376/chr2:41642;chr2/63286681/63286768/chr2:41644;chr2/66653857/
66654048/chr2:42793;chr2/66653857/66654048/chr2:42793;chr2/66666351/66666754/chr2:42805;chr2/666663
51/66666754/chr2:42805;chr2/66666351/66666754/chr2:42805;chr2/66672408/66672922/chr2:42814;chr2/666
73028/66673388/chr2:42815;chr2/66673028/66673388/chr2:42815;chr2/66673566/66673729/chr2:42816;chr2/
69001814/69001933/chr2:43990;chr2/71123628/71124082/chr2:45169;chr2/71131120/71132341/chr2:45180;c
hr2/71131120/71132341/chr2:45180;chr2/71134507/71134776/chr2:45187;chr2/72371181/72371262/chr2:4572
9;chr2/74642004/74642321/chr2:46548;chr2/74642004/74642321/chr2:46548;chr2/74642571/74642732/chr2:4
6549;chr2/74726584/74726959/chr2:46585;chr2/74726584/74726959/chr2:46585;chr2/74742186/74742361/chr
2:46600;chr2/85811403/85811635/chr2:55658;chr2/89064603/89064923/chr2:57119;chr2/97193044/97193288/
chr2:59984;chr2/97193044/97193288/chr2:59984;chr2/98698225/98698488/chr2:60664;chr2/102804257/10280
4518/chr2:62517;chr2/105461091/105461164/chr2:65581;chr2/105472285/105472476/chr2:65591;chr2/111180
659/111180875/chr2:69784;chr2/112238344/112238562/chr2:70175;chr2/118982055/118982431/chr2:74873;c
hr2/119599436/119599993/chr2:75438;chr2/119599436/119599993/chr2:75438;chr2/119599436/119599993/ch
r2:75438;chr2/119602556/119602749/chr2:75443;chr2/127413759/127414118/chr2:82885;chr2/131674028/131
674174/chr2:86273;chr2/131721230/131721412/chr2:86315;chr2/131721230/131721412/chr2:86315;chr2/1317
92696/131792920/chr2:86384;chr2/131797547/131797851/chr2:86386;chr2/131797547/131797851/chr2:86386;
chr2/137522460/137522886/chr2:90583;chr2/137523350/137523663/chr2:90586;chr2/137523350/137523663/
chr2:90586;chr2/157185658/157185890/chr2:104090;chr2/162273294/162273350/chr2:106478;chr2/16228342
0/162284088/chr2:106503;chr2/162284279/162284774/chr2:106504;chr2/162284279/162284774/chr2:106504;
chr2/170625080/170625211/chr2:110300;chr2/171569569/171570071/chr2:110660;chr2/171572958/17157356
1/chr2:110663;chr2/171572958/171573561/chr2:110663;chr2/171572958/171573561/chr2:110663;chr2/172965
944/172966155/chr2:111138;chr2/172972755/172973021/chr2:111141;chr2/175191910/175192084/chr2:11200
6;chr2/175193753/175193890/chr2:112011;chr2/175195757/175196120/chr2:112016;chr2/175198572/1751990
64/chr2:112021;chr2/175198572/175199064/chr2:112021;chr2/175208701/175208893/chr2:112040;chr2/17693
1542/176931823/chr2:112960;chr2/176931542/176931823/chr2:112960;chr2/176946935/176947014/chr2:1129
81;chr2/176946935/176947014/chr2:112981;chr2/176956625/176956956/chr2:112993;chr2/176964833/176964
945/chr2:113005;chr2/176965226/176965318/chr2:113007;chr2/176973451/176973569/chr2:113020;chr2/1769
82334/176982522/chr2:113036;chr2/177016477/177016749/chr2:113072;chr2/177016477/177016749/chr2:113
072;chr2/177017049/177017266/chr2:113074;chr2/177024768/177024991/chr2:113083;chr2/177036408/17703
6551/chr2:113096;chr2/177036806/177037159/chr2:113097;chr2/177054348/177054557/chr2:113117;chr2/177
873532/177873742/chr2:113554;chr2/182546412/182546709/chr2:116545;chr2/200327235/200327462/chr2:12
4591;chr2/200327235/200327462/chr2:124591;chr2/200333691/200333800/chr2:124599;chr2/208989159/2089
89543/chr2:127877;chr2/219738085/219738741/chr2:132890;chr2/219857695/219857761/chr2:132992;chr2/22
0299712/220299794/chr2:133332;chr2/220313306/220313447/chr2:133352;chr2/220361577/220361845/chr2:1
33406;chr2/220493804/220494007/chr2:133483;chr2/233925072/233925317/chr2:141676;chr2/236579854/236
580385/chr2:143448;chr2/236962012/236962127/chr2:143566;chr2/237067694/237068149/chr2:143602;chr2/2
37068816/237069041/chr2:143605;chr2/237071845/237072124/chr2:143612;chr2/237072390/237072589/chr2:
143614;chr2/237081673/237081834/chr2:143623;chr2/238864628/238865041/chr2:145217;chr2/239072322/23
9072437/chr2:145320;chr2/240302575/240302644/chr2:146577;chr20/592495/592583/chr20:366;chr20/825633/
826365/chr20:567;chr20/825633/826365/chr20:567;chr20/3073947/3074286/chr20:2072;chr20/3073947/30742
86/chr20:2072;chr20/3204041/3204138/chr20:2128;chr20/17840247/17840428/chr20:11743;chr20/20433026/2
0433366/chr20:13300;chr20/21082149/21082215/chr20:13769;chr20/21687581/21687936/chr20:14217;chr20/2
1687581/21687936/chr20:14217;chr20/23066774/23067283/chr20:15385;chr20/23066774/23067283/chr20:153
85;chr20/23636683/23636783/chr20:15653;chr20/31116249/31116419/chr20:19124;chr20/32856719/32857305/
chr20:20375;chr20/34188455/34188891/chr20:20861;chr20/35490145/35491178/chr20:21574;chr20/36012595/
36012923/chr20:21782;chr20/39311379/39311636/chr20:24463;chr20/42744689/42744736/chr20:27929;chr20/
42744873/42744943/chr20:27930;chr20/43279673/43279869/chr20:28494;chr20/44686589/44686643/chr20:2
9750;chr20/44880014/44880139/chr20:30033;chr20/49307683/49308050/chr20:33326;chr20/49307683/493080
50/chr20:33326;chr20/52789941/52790002/chr20:35507;chr20/55200052/55200698/chr20:37915;chr20/552000
52/55200698/chr20:37915;chr20/55200052/55200698/chr20:37915;chr20/55202028/55202768/chr20:37919;chr
20/55202028/55202768/chr20:37919;chr20/55532842/55532860/chr20:38128;chr20/55965227/55965866/chr20:
38359;chr20/55965227/55965866/chr20:38359;chr20/56196092/56196287/chr20:38599;chr20/57224517/5722
5315/chr20:40041;chr20/57224517/57225315/chr20:40041;chr20/57224517/57225315/chr20:40041;chr20/5863
0953/58631215/chr20:41275;chr20/59828727/59828805/chr20:42845;chr20/61585426/61586109/chr20:45350;
chr20/61992028/61992488/chr20:46132;chr21/32546304/32546471/chr21:16832;chr21/38070378/38070671/ch
r21:21460;chr21/38076979/38077067/chr21:21468;chr21/38378456/38378600/chr21:21622;chr21/42218718/42
219076/chr21:24058;chr21/43145582/43146564/chr21:24587;chr21/44819059/44819973/chr21:26123;chr21/44
819059/44819973/chr21:26123;chr21/45246251/45246468/chr21:26382;chr21/45577321/45577994/chr21:2658
8;chr21/45577321/45577994/chr21:26588;chr21/45592714/45592967/chr21:26625;chr21/45592714/45592967/
chr21:26625;chr21/45627357/45627417/chr21:26687;chr21/45770297/45770337/chr21:26879;chr21/45770637/
45770704/chr21:26880;chr21/46126288/46126559/chr21:27407;chr21/46126288/46126559/chr21:27407;chr21/
46331605/46331714/chr21:27573;chr21/46334321/46334574/chr21:27580;chr21/47971598/47971831/chr21:30
067;chr22/19760482/19760564/chr22:2520;chr22/20780347/20780473/chr22:3197;chr22/23523932/23524438/
chr22:5588;chr22/23523932/23524438/chr22:5588;chr22/30661877/30662289/chr22:11999;chr22/30662646/30
663433/chr22:12000;chr22/33271212/33271346/chr22:13703;chr22/35748604/35748744/chr22:16424;chr22/35
876979/35877087/chr22:16512;chr22/37962038/37962114/chr22:18791;chr22/38572642/38572698/chr22:1919
5;chr22/40058029/40058457/chr22:20645;chr22/40058029/40058457/chr22:20645;chr22/42316277/42316352/
chr22:22245;chr22/42828172/42828446/chr22:22503;chr22/43524334/43524374/chr22:22910;chr22/45236689/
45236699/chr22:25804;chr22/46307842/46307986/chr22:26661;chr22/46362417/46362750/chr22:26698;chr22/
46367863/46368544/chr22:26710;chr22/46367863/46368544/chr22:26710;chr22/46367863/46368544/chr22:26
710;chr22/47070140/47070284/chr22:27244;chr22/47070407/47070938/chr22:27245;chr22/47170472/4717063
0/chr22:27297;chr22/50064671/50065042/chr22:32285;chr22/50201718/50201897/chr22:32524;chr22/5049719
0/50497359/chr22:32806;chr22/50623245/50623586/chr22:33035;chr22/50623245/50623586/chr22:33035;chr2
2/50986812/50986985/chr22:33343;chr22/50987102/50987681/chr22:33344;chr22/50987102/50987681/chr22:
33344;chr22/51042167/51042182/chr22:33382;chr22/51042483/51042635/chr22:33384;chr3/12910074/129105
05/chr3:11147;chr3/13456076/13456222/chr3:11629;chr3/32501521/32501675/chr3:21388;chr3/40657309/406
57378/chr3:25372;chr3/42947455/42947990/chr3:26304;chr3/48520598/48520813/chr3:29151;chr3/50313752/
50313821/chr3:29988;chr3/50378590/50378763/chr3:30022;chr3/52529185/52529620/chr3:31545;chr3/525291
85/52529620/chr3:31545;chr3/55517100/55517495/chr3:32653;chr3/56950279/56950401/chr3:33283;chr3/623
57136/62357293/chr3:35276;chr3/62358179/62358236/chr3:35278;chr3/63264002/63264099/chr3:36189;chr3/
65535076/65535366/chr3:37424;chr3/66848640/66848880/chr3:37910;chr3/67706043/67706567/chr3:38421;c
hr3/71275877/71276181/chr3:40964;chr3/87138182/87138575/chr3:53879;chr3/87138182/87138575/chr3:5387
9;chr3/109055832/109056025/chr3:66444;chr3/127795108/127796134/chr3:77376;chr3/127795108/127796134/
chr3:77376;chr3/127795108/127796134/chr3:77376;chr3/128274652/128274738/chr3:77592;chr3/129296978/
129297230/chr3:78142;chr3/129312876/129313022/chr3:78152;chr3/129693906/129694016/chr3:78292;chr3/1
33393092/133393676/chr3:80380;chr3/133393092/133393676/chr3:80380;chr3/133393092/133393676/chr3:80
380;chr3/138658264/138658703/chr3:82947;chr3/138658264/138658703/chr3:82947;chr3/138658812/1386591
05/chr3:82948;chr3/138658812/138659105/chr3:82948;chr3/147108531/147108856/chr3:88574;chr3/14711152
9/147111637/chr3:88580;chr3/147114049/147114194/chr3:88585;chr3/147127937/147127985/chr3:88606;chr3/
147128489/147128669/chr3:88609;chr3/147139245/147139324/chr3:88629;chr3/157812151/157812304/chr3:
93922;chr3/157821232/157821290/chr3:93935;chr3/171175712/171176140/chr3:101703;chr3/184319330/1843
19689/chr3:108851;chr3/184405514/184405563/chr3:108896;chr3/187676428/187676667/chr3:111184;chr3/19
2125798/192125964/chr3:113516;chr3/192127390/192127461/chr3:113520;chr3/194117547/194118422/chr3:1
14718;chr3/194117547/194118422/chr3:114718;chr3/194117547/194118422/chr3:114718;chr3/195583592/195
583698/chr3:115500;chr3/195886887/195887205/chr3:115743;chr3/196367454/196367940/chr3:116035;chr4/7
79344/779699/chr4:501;chr4/780353/780533/chr4:503;chr4/780353/780533/chr4:503;chr4/1049831/1049922/c
hr4:802;chr4/1210537/1211061/chr4:1058;chr4/1210537/1211061/chr4:1058;chr4/1398849/1399019/chr4:1184;
chr4/1773190/1773264/chr4:1786;chr4/1773616/1773799/chr4:1788;chr4/2292691/2292975/chr4:2184;chr4/48
59771/4860060/chr4:4199;chr4/4863382/4863400/chr4:4207;chr4/4869018/4869038/chr4:4217;chr4/5710267/5
710387/chr4:5397;chr4/6666045/6666167/chr4:6989;chr4/7195168/7195465/chr4:7409;chr4/8859124/8859261/
chr4:9784;chr4/8859124/8859261/chr4:9784;chr4/8864739/8864795/chr4:9796;chr4/8866180/8866258/chr4:97
99;chr4/8869267/8869307/chr4:9801;chr4/9783347/9783507/chr4:11142;chr4/10589431/10589606/chr4:11493;
chr4/13545251/13545542/chr4:14438;chr4/20256325/20256382/chr4:18872;chr4/21305940/21306025/chr4:198
92;chr4/24801473/24801623/chr4:22965;chr4/24801473/24801623/chr4:22965;chr4/24801727/24802065/chr4:
22966;chr4/24801727/24802065/chr4:22966;chr4/26275411/26275517/chr4:23558;chr4/41747656/41747785/c
hr4:36276;chr4/48493130/48493313/chr4:42704;chr4/48987795/48987835/chr4:42836;chr4/56659626/5665998
4/chr4:44965;chr4/85417611/85418301/chr4:61218;chr4/85418447/85418583/chr4:61219;chr4/85418687/8541
8746/chr4:61220;chr4/93226624/93226785/chr4:64606;chr4/102712009/102712321/chr4:69608;chr4/10271200
9/102712321/chr4:69608;chr4/111531751/111533022/chr4:73577;chr4/111531751/111533022/chr4:73577;chr4/
111531751/111533022/chr4:73577;chr4/111533322/111533386/chr4:73579;chr4/111536462/111536678/chr4:
73586;chr4/111536860/111537229/chr4:73587;chr4/111539436/111539906/chr4:73593;chr4/111540024/11154
0345/chr4:73594;chr4/111540024/111540345/chr4:73594;chr4/111540589/111540656/chr4:73596;chr4/111541
822/111541877/chr4:73600;chr4/111542062/111542380/chr4:73601;chr4/111554905/111554987/chr4:73618;c
hr4/111555101/111555502/chr4:73619;chr4/113432194/113432240/chr4:74602;chr4/113432450/113432550/ch
r4:74603;chr4/140656581/140657036/chr4:88873;chr4/144621470/144621591/chr4:90504;chr4/147560126/147
560227/chr4:91488;chr4/147560922/147561454/chr4:91490;chr4/147560922/147561454/chr4:91490;chr4/1547
10713/154710960/chr4:94568;chr4/155663868/155663983/chr4:95583;chr4/155664152/155664237/chr4:95584;
chr4/155666318/155666451/chr4:95586;chr4/169799143/169799621/chr4:103089;chr4/169799143/169799621/
chr4:103089;chr4/174422105/174422220/chr4:105773;chr4/174427714/174428328/chr4:105779;chr4/1744277
14/174428328/chr4:105779;chr4/174429805/174430859/chr4:105786;chr4/174429805/174430859/chr4:105786;
chr4/174437816/174437949/chr4:105796;chr4/174438994/174439178/chr4:105801;chr4/174445205/17444534
9/chr4:105812;chr4/174446409/174446459/chr4:105816;chr4/174452450/174452494/chr4:105826;chr4/183061
804/183062036/chr4:113059;chr4/183061804/183062036/chr4:113059;chr4/184828402/184828628/chr4:11385
4;chr4/186559232/186560504/chr4:114704;chr4/188918071/188918353/chr4:116452;chr4/188953026/1889532
83/chr4:116484;chr5/501955/502073/chr5:462;chr5/1851493/1851667/chr5:2487;chr5/1878105/1879524/chr5:2
522;chr5/1881584/1882309/chr5:2530;chr5/1881584/1882309/chr5:2530;chr5/1881584/1882309/chr5:2530;chr
5/2738572/2739312/chr5:3972;chr5/2740803/2741046/chr5:3973;chr5/2740803/2741046/chr5:3973;chr5/27408
03/2741046/chr5:3973;chr5/2743537/2743740/chr5:3982;chr5/2748314/2748924/chr5:3990;chr5/2748314/2748
924/chr5:3990;chr5/2749090/2749714/chr5:3991;chr5/2749090/2749714/chr5:3991;chr5/3591539/3591793/chr
5:5340;chr5/3596430/3596580/chr5:5353;chr5/3596430/3596580/chr5:5353;chr5/31194155/31194326/chr5:298
22;chr5/40680880/40681286/chr5:34395;chr5/40681858/40682027/chr5:34397;chr5/42951163/42951584/chr5:
35253;chr5/42952279/42952433/chr5:35255;chr5/42952279/42952433/chr5:35255;chr5/42952583/42952755/c
hr5:35256;chr5/50685285/50685600/chr5:38137;chr5/50685285/50685600/chr5:38137;chr5/54518882/5451943
2/chr5:40420;chr5/55117709/55117762/chr5:40593;chr5/60922160/60922380/chr5:42941;chr5/63801781/6380
1823/chr5:44959;chr5/63802221/63802341/chr5:44960;chr5/72526411/72527038/chr5:47662;chr5/72528040/7
2528709/chr5:47664;chr5/72594128/72594196/chr5:47695;chr5/72677124/72677354/chr5:47751;chr5/7267762
8/72677905/chr5:47752;chr5/72677628/72677905/chr5:47752;chr5/72678016/72678419/chr5:47753;chr5/7273
1821/72732044/chr5:47799;chr5/72732779/72732854/chr5:47802;chr5/76924024/76924300/chr5:49906;chr5/7
6933446/76933493/chr5:49916;chr5/77140756/77140889/chr5:50032;chr5/77253377/77253652/chr5:50094;chr
5/77253832/77254047/chr5:50095;chr5/77253832/77254047/chr5:50095;chr5/87955564/87955812/chr5:55500;
chr5/87968708/87968844/chr5:55512;chr5/92906093/92906850/chr5:59422;chr5/92906093/92906850/chr5:594
22;chr5/92908237/92908299/chr5:59425;chr5/92923512/92924029/chr5:59436;chr5/92923512/92924029/chr5:
59436;chr5/92923512/92924029/chr5:59436;chr5/92930529/92930781/chr5:59446;chr5/100239964/100240164/
chr5:63724;chr5/122433878/122433967/chr5:74156;chr5/122434104/122434128/chr5:74157;chr5/122434243/
122434303/chr5:74158;chr5/132147985/132148073/chr5:78096;chr5/132149854/132150238/chr5:78097;chr5/1
32149854/132150238/chr5:78097;chr5/132161113/132161911/chr5:78104;chr5/132161113/132161911/chr5:78
104;chr5/132161113/132161911/chr5:78104;chr5/132161113/132161911/chr5:78104;chr5/132161113/1321619
11/chr5:78104;chr5/133449110/133449541/chr5:78662;chr5/134363102/134364716/chr5:78998;chr5/13436310
2/134364716/chr5:78998;chr5/134363102/134364716/chr5:78998;chr5/134363102/134364716/chr5:78998;chr5/
134363102/134364716/chr5:78998;chr5/134363102/134364716/chr5:78998;chr5/134363102/134364716/chr5:
78998;chr5/134363102/134364716/chr5:78998;chr5/134364837/134365262/chr5:78999;chr5/134366914/13436
7436/chr5:79006;chr5/134376713/134376745/chr5:79018;chr5/134385819/134386368/chr5:79020;chr5/137795
098/137795305/chr5:81306;chr5/138850474/138850701/chr5:81647;chr5/139089542/139090010/chr5:81772;c
hr5/139144057/139144077/chr5:81802;chr5/139525719/139526053/chr5:82012;chr5/140306412/140306894/ch
r5:82369;chr5/140870906/140872485/chr5:82848;chr5/140870906/140872485/chr5:82848;chr5/140870906/140
872485/chr5:82848;chr5/140870906/140872485/chr5:82848;chr5/140870906/140872485/chr5:82848;chr5/1408
70906/140872485/chr5:82848;chr5/145717244/145717266/chr5:85426;chr5/145722488/145722602/chr5:85433;
chr5/148620215/148620253/chr5:87233;chr5/149546507/149546945/chr5:87652;chr5/149588968/149589167/
chr5:87673;chr5/150226201/150226446/chr5:87968;chr5/150226201/150226446/chr5:87968;chr5/153856015/1
53856075/chr5:90240;chr5/153858894/153858919/chr5:90246;chr5/153872975/153873090/chr5:90265;chr5/17
0737535/170738058/chr5:104900;chr5/170738774/170738886/chr5:104902;chr5/170741270/170741684/chr5:1
04910;chr5/170742528/170743369/chr5:104913;chr5/170743480/170744113/chr5:104914;chr5/171605252/171
605555/chr5:105373;chr5/172661014/172661117/chr5:106047;chr5/172663078/172663257/chr5:106053;chr5/1
74035906/174036064/chr5:107010;chr5/175560217/175560364/chr5:108580;chr5/176784998/176785181/chr5:
109677;chr5/176935213/176935464/chr5:109846;chr5/177434365/177434476/chr5:110299;chr5/177667820/17
7667909/chr5:110516;chr5/177988973/177989166/chr5:110736;chr5/178004175/178004617/chr5:110758;chr5/
178004175/178004617/chr5:110758;chr5/178420360/178420637/chr5:110987;chr5/180230432/180231193/chr
5:112498;chr6/392448/392484/chr6:258;chr6/1383826/1383865/chr6:1183;chr6/1601838/1601972/chr6:1422;c
hr6/1624759/1624977/chr6:1443;chr6/4079052/4079410/chr6:2565;chr6/6002471/6002605/chr6:3507;chr6/939
9170/9399321/chr6:5424;chr6/10113475/10113737/chr6:5604;chr6/10382027/10382451/chr6:5705;chr6/10382
027/10382451/chr6:5705;chr6/10398366/10398636/chr6:5743;chr6/10421512/10421572/chr6:5756;chr6/10425
307/10425542/chr6:5767;chr6/10720335/10720454/chr6:5918;chr6/10883079/10883209/chr6:5972;chr6/10883
702/10883894/chr6:5973;chr6/17016236/17016470/chr6:9094;chr6/20320017/20320581/chr6:11075;chr6/2725
1406/27251585/chr6:15015;chr6/27251740/27252023/chr6:15016;chr6/28741414/28741592/chr6:15820;chr6/2
8743522/28743843/chr6:15826;chr6/28743522/28743843/chr6:15826;chr6/28747803/28747995/chr6:15835;chr
6/29760089/29760181/chr6:17122;chr6/29894371/29894467/chr6:17315;chr6/30420999/30421116/chr6:17801;
chr6/32116930/32117018/chr6:19468;chr6/32813440/32813590/chr6:20282;chr6/35108801/35109129/chr6:220
71;chr6/35480055/35480141/chr6:22274;chr6/36737488/36737623/chr6:22829;chr6/41515716/41516104/chr6:
25691;chr6/41515716/41516104/chr6:25691;chr6/41515716/41516104/chr6:25691;chr6/41516270/41516539/c
hr6:25692;chr6/42335111/42335253/chr6:26100;chr6/44529077/44529148/chr6:27193;chr6/50787947/5078808
2/chr6:29961;chr6/50791110/50791617/chr6:29965;chr6/56716314/56716535/chr6:32402;chr6/84417571/8441
7763/chr6:49979;chr6/100903446/100903662/chr6:61889;chr6/100905984/100906245/chr6:61896;chr6/106429
435/106429672/chr6:64464;chr6/106434053/106434088/chr6:64468;chr6/107955163/107955625/chr6:65044;c
hr6/108437998/108438266/chr6:65256;chr6/108439985/108441283/chr6:65261;chr6/108439985/108441283/ch
r6:65261;chr6/108488593/108489118/chr6:65301;chr6/108488593/108489118/chr6:65301;chr6/108489236/108
489404/chr6:65302;chr6/108489620/108489668/chr6:65304;chr6/137809770/137810125/chr6:78388;chr6/1502
85936/150286136/chr6:83626;chr6/152002855/152003072/chr6:84423;chr6/163670578/163670807/chr6:93227;
chr6/163756788/163756921/chr6:93257;chr6/164051683/164051908/chr6:93382;chr6/166073897/166074010/
chr6:94398;chr6/168107103/168107373/chr6:97051;chr6/169572592/169572700/chr6:98184;chr6/170338436/1
70338749/chr6:98738;chr6/170338436/170338749/chr6:98738;chr6/170403023/170403277/chr6:98835;chr6/17
0580898/170581131/chr6:99039;chr7/150008/150194/chr7:188;chr7/331078/331128/chr7:465;chr7/641336/641
390/chr7:998;chr7/644124/644459/chr7:1002;chr7/644124/644459/chr7:1002;chr7/751335/752464/chr7:1111;c
hr7/1550454/1550554/chr7:2231;chr7/1552571/1552941/chr7:2235;chr7/1959733/1960285/chr7:2971;chr7/205
9280/2059501/chr7:3037;chr7/2059710/2060006/chr7:3038;chr7/2289078/2289150/chr7:3159;chr7/2548120/25
48211/chr7:3399;chr7/2550517/2550795/chr7:3404;chr7/3134486/3134688/chr7:4224;chr7/4304772/4305060/c
hr7:5076;chr7/4831750/4832626/chr7:5827;chr7/4848636/4849016/chr7:5865;chr7/12151500/12151663/chr7:1
1942;chr7/12610688/12610816/chr7:12112;chr7/19184554/19184738/chr7:16024;chr7/20824268/20824384/chr
7:17442;chr7/20824268/20824384/chr7:17442;chr7/23245947/23246055/chr7:18981;chr7/24323291/24323395/
chr7:19618;chr7/24325010/24325141/chr7:19622;chr7/25892451/25892715/chr7:20791;chr7/25898244/258992
81/chr7:20799;chr7/25898244/25899281/chr7:20799;chr7/25898244/25899281/chr7:20799;chr7/27199643/272
00193/chr7:21616;chr7/27204937/27204950/chr7:21623;chr7/27205783/27206164/chr7:21625;chr7/27205783/
27206164/chr7:21625;chr7/27214295/27214382/chr7:21636;chr7/27252771/27253381/chr7:21670;chr7/272602
29/27260528/chr7:21676;chr7/27260229/27260528/chr7:21676;chr7/27279058/27279119/chr7:21687;chr7/272
84639/27284889/chr7:21698;chr7/27284639/27284889/chr7:21698;chr7/27284639/27284889/chr7:21698;chr7/
27288961/27289408/chr7:21705;chr7/27290903/27291485/chr7:21709;chr7/27290903/27291485/chr7:21709;c
hr7/29606046/29606319/chr7:22817;chr7/35294861/35295431/chr7:26623;chr7/35296875/35297026/chr7:2662
7;chr7/35297250/35297364/chr7:26628;chr7/35297698/35298216/chr7:26630;chr7/35298542/35298706/chr7:2
6632;chr7/35494396/35494506/chr7:26792;chr7/37392844/37393221/chr7:28387;chr7/45690753/45690819/chr
7:33273;chr7/47608222/47608452/chr7:34502;chr7/56297481/56297660/chr7:43272;chr7/64349675/64350015/
chr7:47203;chr7/65878615/65878773/chr7:48104;chr7/71800646/71800933/chr7:55088;chr7/71801344/718015
41/chr7:55090;chr7/71801344/71801541/chr7:55090;chr7/71801799/71802093/chr7:55092;chr7/71801799/718
02093/chr7:55092;chr7/73121630/73121821/chr7:56155;chr7/73183158/73184204/chr7:56212;chr7/73508288/
73508573/chr7:56510;chr7/73508677/73509186/chr7:56511;chr7/86274132/86274274/chr7:65151;chr7/862741
32/86274274/chr7:65151;chr7/94023566/94023795/chr7:70740;chr7/96650061/96650101/chr7:71790;chr7/966
50428/96650566/chr7:71792;chr7/100028259/100028424/chr7:74817;chr7/100844956/100845521/chr7:75501;
chr7/100844956/100845521/chr7:75501;chr7/116964411/116964725/chr7:83336;chr7/121946918/121947079/c
hr7:86344;chr7/121957163/121957214/chr7:86360;chr7/127671158/127671217/chr7:89761;chr7/127671158/12
7671217/chr7:89761;chr7/127671581/127672237/chr7:89763;chr7/127671581/127672237/chr7:89763;chr7/127
671581/127672237/chr7:89763;chr7/127672383/127672851/chr7:89764;chr7/127672383/127672851/chr7:8976
4;chr7/128555660/128555728/chr7:90426;chr7/129781009/129781205/chr7:90966;chr7/132261368/132261547/
chr7:92851;chr7/142495145/142495246/chr7:98662;chr7/149119904/149120359/chr7:104170;chr7/149119904/
149120359/chr7:104170;chr7/149745077/149745159/chr7:104739;chr7/153445194/153445394/chr7:108035;c
hr7/153583530/153583710/chr7:108224;chr7/153748796/153748893/chr7:108489;chr7/153749894/153750278/
chr7:108492;chr7/154002021/154002196/chr7:108884;chr7/154705814/154705867/chr7:109868;chr7/1551670
37/155167117/chr7:110332;chr7/155250298/155250363/chr7:110491;chr7/155258829/155258985/chr7:110504;
chr7/155556480/155556793/chr7:110841;chr7/155595704/155595803/chr7:110879;chr7/155598690/15559974
6/chr7:110882;chr7/155598690/155599746/chr7:110882;chr7/156796810/156796867/chr7:112365;chr7/156813
643/156813832/chr7:112396;chr7/157485363/157485649/chr7:113322;chr7/157486426/157486717/chr7:11332
6;chr7/157486426/157486717/chr7:113326;chr7/158110722/158110752/chr7:114517;chr7/158362621/1583627
44/chr7:114937;chr7/158362621/158362744/chr7:114937;chr7/158505175/158505367/chr7:114992;chr8/19504
76/1950657/chr8:3057;chr8/8559885/8559991/chr8:12234;chr8/8654846/8654935/chr8:12313;chr8/10586613/1
0587079/chr8:14495;chr8/10588583/10589358/chr8:14496;chr8/10588583/10589358/chr8:14496;chr8/1750911
1/17509337/chr8:22638;chr8/19797537/19797821/chr8:23816;chr8/20092455/20092607/chr8:24179;chr8/2190
5524/21905755/chr8:26910;chr8/21905524/21905755/chr8:26910;chr8/25898101/25898219/chr8:30839;chr8/2
5900639/25900766/chr8:30847;chr8/25907047/25907075/chr8:30858;chr8/27221306/27221473/chr8:32212;chr
8/37557235/37557369/chr8:43546;chr8/38412086/38412230/chr8:44019;chr8/41424341/41425161/chr8:46141;
chr8/41424341/41425161/chr8:46141;chr8/41624189/41624427/chr8:46264;chr8/54789694/54789895/chr8:556
76;chr8/54791824/54791953/chr8:55682;chr8/65282149/65282304/chr8:65459;chr8/65282149/65282304/chr8:
65459;chr8/65282839/65282980/chr8:65462;chr8/65286067/65286267/chr8:65468;chr8/65286067/65286267/c
hr8:65468;chr8/70982068/70982262/chr8:70720;chr8/80804060/80804359/chr8:80237;chr8/80804060/8080435
9/chr8:80237;chr8/96193567/96193686/chr8:92031;chr8/97156629/97157376/chr8:92621;chr8/97156629/9715
7376/chr8:92621;chr8/97170306/97170403/chr8:92641;chr8/99954431/99954678/chr8:94384;chr8/99959773/9
9959954/chr8:94393;chr8/99960243/99962202/chr8:94395;chr8/99960243/99962202/chr8:94395;chr8/9996024
3/99962202/chr8:94395;chr8/99960243/99962202/chr8:94395;chr8/99985631/99986981/chr8:94413;chr8/9998
5631/99986981/chr8:94413;chr8/99985631/99986981/chr8:94413;chr8/99985631/99986981/chr8:94413;chr8/1
01118020/101118645/chr8:94672;chr8/101118020/101118645/chr8:94672;chr8/102236112/102236533/chr8:95
187;chr8/103750882/103751038/chr8:95871;chr8/105478815/105478973/chr8:97368;chr8/116679656/1166798
60/chr8:109001;chr8/142045998/142046591/chr8:131588;chr8/142099939/142100219/chr8:131632;chr8/14210
5772/142105871/chr8:131639;chr8/142160199/142160378/chr8:131679;chr8/142216446/142216722/chr8:1317
19;chr8/142216835/142216922/chr8:131720;chr8/142276263/142276522/chr8:131773;chr8/142276263/142276
522/chr8:131773;chr8/143545856/143546249/chr8:134688;chr8/143545856/143546249/chr8:134688;chr8/1435
45856/143546249/chr8:134688;chr8/143545856/143546249/chr8:134688;chr8/144105813/144105864/chr8:135
782;chr8/144598804/144599034/chr8:136515;chr8/144965156/144965351/chr8:136809;chr8/145106040/14510
6315/chr8:136951;chr8/145106953/145107056/chr8:136954;chr8/145725668/145725940/chr8:137231;chr8/145
900685/145901387/chr8:137341;chr8/145900685/145901387/chr8:137341;chr9/971339/971502/chr9:559;chr9/
971339/971502/chr9:559;chr9/1046136/1046387/chr9:629;chr9/1046136/1046387/chr9:629;chr9/14345455/143
46298/chr9:9646;chr9/17906443/17906570/chr9:11721;chr9/17907412/17907457/chr9:11723;chr9/21970692/2
1971184/chr9:14497;chr9/22006179/22006227/chr9:14533;chr9/23820722/23820875/chr9:16719;chr9/3564967
2/35649818/chr9:27462;chr9/36458376/36458935/chr9:27909;chr9/40332435/40332647/chr9:30081;chr9/7097
1904/70972207/chr9:33824;chr9/89517790/89517820/chr9:45940;chr9/90692017/90692063/chr9:46536;chr9/9
2098876/92099282/chr9:47340;chr9/93601605/93601852/chr9:48090;chr9/93605351/93605452/chr9:48093;chr
9/93925345/93925540/chr9:48226;chr9/95821836/95821957/chr9:49201;chr9/96030882/96031112/chr9:49324;
chr9/96108466/96109062/chr9:49358;chr9/98315341/98315575/chr9:50909;chr9/99482110/99482542/chr9:517
30;chr9/110249659/110249822/chr9:60417;chr9/124981875/124982017/chr9:71533;chr9/126135749/12613603
4/chr9:72291;chr9/126182928/126183193/chr9:72320;chr9/126775129/126775228/chr9:72476;chr9/126776099/
126776220/chr9:72480;chr9/126776099/126776220/chr9:72480;chr9/126777283/126777853/chr9:72484;chr9/
126778239/126778747/chr9:72485;chr9/126779162/126779369/chr9:72487;chr9/128651477/128651756/chr9:7
3447;chr9/129377596/129377653/chr9:74268;chr9/129386285/129386431/chr9:74290;chr9/130545182/130545
693/chr9:75047;chr9/131465499/131465770/chr9:75689;chr9/132099165/132099614/chr9:76050;chr9/1320991
65/132099614/chr9:76050;chr9/132650647/132651025/chr9:76569;chr9/132650647/132651025/chr9:76569;chr
9/134128101/134128615/chr9:77644;chr9/134148417/134149187/chr9:77669;chr9/134150975/134151518/chr9:
77677;chr9/134429947/134430489/chr9:77880;chr9/134429947/134430489/chr9:77880;chr9/134429947/1344
30489/chr9:77880;chr9/134465858/134466099/chr9:77900;chr9/136150213/136150486/chr9:79817;chr9/13671
0220/136710361/chr9:80713;chr9/137185428/137185962/chr9:81257;chr9/137288189/137288710/chr9:81367;
chr9/138136050/138136084/chr9:83477;chr9/138999163/138999618/chr9:85122;chr9/139002578/139002859/c
hr9:85129;chr9/139002578/139002859/chr9:85129;chr9/139428429/139428610/chr9:85664;chr9/139514100/13
9514472/chr9:85767;chr9/139522722/139522800/chr9:85784;chr9/139715663/139716114/chr9:86060;chr9/139
880320/139880622/chr9:86173;chr9/139880320/139880622/chr9:86173;chr9/139892507/139892724/chr9:8619
4;chrX/9733543/9734032/chrX:7325;chrX/9733543/9734032/chrX:7325;chrX/11445215/11445879/chrX:8252;ch
rX/17673590/17674072/chrX:13316;chrX/17674183/17674448/chrX:13317;chrX/17674183/17674448/chrX:1331
7;chrX/25029910/25030303/chrX:18683;chrX/39865028/39865263/chrX:32853;chrX/39865028/39865263/chrX:
32853;chrX/39865028/39865263/chrX:32853;chrX/39868827/39868988/chrX:32862;chrX/39943287/39943396/
chrX:32911;chrX/39949200/39949896/chrX:32923;chrX/39949200/39949896/chrX:32923;chrX/40027402/40027
583/chrX:32995;chrX/40027402/40027583/chrX:32995;chrX/51486750/51486844/chrX:41006;chrX/82763606/8
2763847/chrX:63554;chrX/102862629/102863551/chrX:78770;chrX/139846861/139847316/chrX:106698;chrX/1
44903082/144903254/chrX:112617

TABLE 3
1/chr2:2233:chr2:74875400-74875253:2;2/chr17:3789:chr17:76127734-76127596:2;3/chr17:3789:chr17:761277
34-76127596:1;4/chr13:1108:chr13:102569075-102568913:2;5/chr15:1558:chr15:76639811-76639638:2;6/chr3:
2471:chr3:129693768-129693603:2;7/chr3:2471:chr3:129693768-129693603:1;8/chr20:476:chr20:20346167-20
345995:2;9/chr20:476:chr20:20346167-20345995:1;10/chrX:666:chrX:39865012-39864870:2;11/chrX:666:chrX:
39865012-39864870:1;12/chr6:107:chr6:1394171-1393998:2;13/chr6:107:chr6:1394171-1393998:1;14/chr10:36
52:chr10:126841036-126840898:2;15/chr5:511:chr5:1886280-1886110:2;16/chr5:511:chr5:1886280-1886110:1;
17/chr10:2358:chr10:90343146-90342980:2;18/chr10:2358:chr10:90343146-90342980:1;19/chr7:1085:chr7:492
3213-4923087:2;20/chr7:1085:chr7:4923213-4923087:1;21/chr22:369:chr22:22863239-22863073:2;22/chr22:36
9:chr22:22863239-22863073:1;23/chr2:4945:chr2:231693435-231693284:2;24/chr1:6929:chr1:228604223-2286
04052:2;25/chr1:6929:chr1:228604223-228604052:1;26/chr1:135:chr1:1118365-1118237:2;27/chr2:4945:chr2:2
31693435-231693284:1;28/chr6:1534:chr6:41516262-41516105:2;29/chr6:1534:chr6:41516262-41516105:1;30/
chr5:1172:chr5:15500318-15500170:2;31/chr5:1172:chr5:15500318-15500170:1;32/chr21:100:chr21:27945341-
27945166:2;33/chr21:100:chr21:27945341-27945166:1;34/chr17:4139:chr17:77789328-77789178:2;35/chr17:4
139:chr17:77789328-77789178:1;36/chr2:3049:chr2:112939620-112939458:2;37/chr2:3049:chr2:112939620-11
2939458:1;38/chr2:3000:chr2:110372627-110372460:2;39/chr2:3000:chr2:110372627-110372460:1;40/chr15:2
02:chr15:27018021-27017886:2;41/chr21:214:chr21:34442616-34442458:2;42/chr22:2208:chr22:48972303-489
72136:2;43/chr1:255:chr1:1567854-1567688:2;44/chr1:255:chr1:1567854-1567688:1;45/chr5:2253:chr5:132159
079-132158908:2;46/chr5:2253:chr5:132159079-132158908:1;47/chr4:2621:chr4:158141798-158141647:2;48/c
hr4:2621:chr4:158141798-158141647:1;49/chr15:2502:chr15:101514172-101513998:2;50/chr15:2502:chr15:10
1514172-101513998:1;51/chr16:3215:chr16:77468577-77468407:2;52/chr10:524:chr10:8097895-8097770:2;53/
chr10:524:chr10:8097895-8097770:1;54/chr14:2140:chr14:103011201-103011069:2;55/chr14:2140:chr14:1030
11201-103011069:1;56/chr16:2161:chr16:49316592-49316446:2;57/chr16:2161:chr16:49316592-49316446:1;5
8/chr16:660:chr16:2229286-2229134:2;59/chr16:660:chr16:2229286-2229134:1;60/chr21:158:chr21:32931455-
32931302:2;61/chr21:158:chr21:32931455-32931302:1;62/chr1:1408:chr1:4715062-4714912:2;63/chr1:1408:ch
r1:4715062-4714912:1;64/chr7:2651:chr7:71801536-71801361:2;65/chr18:585:chr18:32847656-32847529:2;66/
chr18:585:chr18:32847656-32847529:1;67/chrX:693:chrX:40005296-40005124:2;68/chr6:384:chr6:10385378-1
0385211:2;69/chr6:384:chr6:10385378-10385211:1;70/chr10:4059:chr10:131763456-131763291:1;71/chr10:23
15:chr10:88127376-88127203:2;72/chr10:4752:chr10:134862727-134862555:2;73/chr10:4752:chr10:13486272
7-134862555:1;74/chr4:1664:chr4:40859206-40859060:2;75/chr20:2210:chr20:59827285-59827128:2;76/chr20:
2210:chr20:59827285-59827128:1;77/chr1:2257:chr1:13840264-13840092:2;78/cg14015706:chr9:132382533-1
32382363:2;79/chr1:3856:chr1:44883461-44883295:2;80/chr1:3856:chr1:44883461-44883295:1;81/chr5:1926:c
hr5:92906491-92906331:2;82/chr5:1926:chr5:92906491-92906331:1;83/chr4:1277:chr4:8873618-8873451:2;84/
chr4:1277:chr4:8873618-8873451:1;85/chr2:1903:chr2:63275748-63275577:2;86/chr2:1903:chr2:63275748-63
275577:1;87/chr13:498:chr13:36045378-36045214:2;88/chr13:498:chr13:36045378-36045214:1;89/chr20:16:ch
r20:590990-590826:2;90/chr20:16:chr20:590990-590826:1;91/chr9:631:chr9:79637979-79637807:2;92/chr9:63
1:chr9:79637979-79637807:1;93/chr12:1125:chr12:52215440-52215276:2;94/chr12:1125:chr12:52215440-5221
5276:1;95/chr5:3695:chr5:178421865-178421739:2;96/chr5:3695:chr5:178421865-178421739:1;97/chr2:5245:c
hr2:237082355-237082229:2;98/chr2:5245:chr2:237082355-237082229:1;99/chr2:2749:chr2:105459384-10545
9226:2;100/chr19:3278:chr19:38886459-38886315:2;101/chr19:3278:chr19:38886459-38886315:1;102/chr5:18
61:chr5:79865947-79865773:2;103/chr5:1861:chr5:79865947-79865773:1;104/chr12:1613:chr12:72666288-726
66160:2;105/chr20:55:chr20:826311-826169:1;106/chr7:4565:chr7:155744739-155744570:2;107/chr7:4565:chr
7:155744739-155744570:1;108/chr11:2492:chr11:65314238-65314112:2;109/chr11:2492:chr11:65314238-6531
4112:1;110/chr22:1852:chr22:46476380-46476222:2;111/chr19:3278:chr19:38886953-38886810:2;112/chr19:3
278:chr19:38886953-38886810:1;113/chr1:3004:chr1:25256952-25256777:2;114/chr1:3004:chr1:25256952-252
56777:1;115/chr2:3107:chr2:115919136-115918980:2;116/chr2:3107:chr2:115919136-115918980:1;117/chr14:
1768:chr14:99712874-99712699:2;118/chr14:1768:chr14:99712874-99712699:1;119/chr1:4393:chr1:65731677-
65731502:2;120/chr1:4393:chr1:65731677-65731502:1;121/chr6:1002:chr6:31466039-31465910:2;122/chr6:10
02:chr6:31466039-31465910:1;123/chr4:1602:chr4:37245666-37245506:2;124/chr4:1602:chr4:37245666-37245
506:1;125/cg20150591:chr7:3341836-3341666:1;126/chr1:3001:chr1:25255666-25255516:2;127/chr1:3001:chr
1:25255666-25255516:1;128/chr3:2471:chr3:129693987-129693820:2;129/chr3:2471:chr3:129693987-1296938
20:1;130/chrX:810:chrX:48685760-48685622:2;131/chrX:810:chrX:48685760-48685622:1;132/chr10:4581:chr1
0:134598205-134598030:2;133/chr10:4581:chr10:134598205-134598030:1;134/chr12:1613:chr12:72666288-72
666160:1;135/chr4:1726:chr4:44449915-44449740:2;136/chr4:1726:chr4:44449915-44449740:1;137/chr12:146
3:chr12:58132085-58131910:2;138/chr12:1463:chr12:58132085-58131910:1;139/chr1:250:chr1:1564969-15647
94:2;140/chr1:250:chr1:1564969-1564794:1;141/chr22:1852:chr22:46476380-46476222:1;142/chr10:1053:chr1
0:30026167-30026042:2;143/chr10:1053:chr10:30026167-30026042:1;144/chr22:1851:chr22:46474973-464748
01:2;145/chr22:1851:chr22:46474973-46474801:1;146/chr1:1191:chr1:3460237-3460068:2;147/chr1:1191:chr1:
3460237-3460068:1;148/chr22:2792:chr22:51136423-51136250:1;149/chr22:1851:chr22:46474900-46474773:2;
150/chr22:1851:chr22:46474900-46474773:1;151/chr1:7109:chr1:234040655-234040480:2;152/chr1:7109:chr1:
234040655-234040480:1;153/chr4:1664:chr4:40859206-40859060:1;154/chr14:2764:chr14:105953705-105953
539:2;155/chr14:2764:chr14:105953705-105953539:1;156/chr1:2257:chr1:13840264-13840092:1;157/cg14015
706:chr9:132382533-132382363:1;158/chr1:253:chr1:1566007-1565832:2;159/chr8:1701:chr8:54163712-54163
569:2;160/chr8:1701:chr8:54163712-54163569:1;161/chr1:253:chr1:1566007-1565832:1;162/chr6:2499:chr6:13
3562528-133562367:2;163/chr6:2499:chr6:133562528-133562367:1;164/chr13:289:chr13:26626335-26626201:
2;165/chr13:289:chr13:26626335-26626201:1;166/chr18:106:chr18:5891051-5890907:2;167/chr18:106:chr18:5
891051-5890907:1;168/chr15:1830:chr15:83952319-83952147:2;169/chr15:1830:chr15:83952319-83952147:1;
170/chr19:4915:chr19:58609688-58609514:2;171/chr19:4915:chr19:58609688-58609514:1;172/chr18:106:chr1
8:5891311-5891142:2;173/chr18:106:chr18:5891311-5891142:1;174/chr2:2749:chr2:105459384-105459226:1;1
75/chr2:1992:chr2:66673393-66673219:2;176/chr2:1992:chr2:66673393-66673219:1;177/chr7:4817:chr7:15747
8260-157478134:2;178/chr7:4817:chr7:157478260-157478134:1;179/chr2:4288:chr2:177053792-177053619:2;
180/chr2:4288:chr2:177053792-177053619:1;181/chr12:1757:chr12:95942574-95942447:2;182/chr12:1757:chr
12:95942574-95942447:1;183/chr13:100:chr13:21296290-21296119:2;184/chr13:100:chr13:21296290-2129611
9:1;185/chr1:3957:chr1:47905180-47905039:2;186/chr1:3957:chr1:47905180-47905039:1;187/chr15:202:chr15:
27018080-27017922:2;188/chr15:202:chr15:27018080-27017922:1;189/chr7:3563:chr7:128809175-128809031:
2;190/chr7:3563:chr7:128809175-128809031:1;191/chr21:948:chr21:45875503-45875340:2;192/chr21:948:chr2
1:45875503-45875340:1;193/chr2:2233:chr2:74875400-74875253:1;194/chr3:1010:chr3:45187734-45187580:2;
195/chr3:1010:chr3:45187734-45187580:1;196/chr10:2022:chr10:77156070-77155895:2;197/chr10:2022:chr10:
77156070-77155895:1;198/chr5:3649:chr5:178017127-178016990:2;199/chr5:3649:chr5:178017127-17801699
0:1;200/chr12:464:chr12:7592967-7592800:2;201/chr12:464:chr12:7592967-7592800:1;202/chr10:784:chr10:16
562465-16562317:2;203/chr10:784:chr10:16562465-16562317:1;204/chr13:1108:chr13:102569075-102568913:
1;205/chr7:3079:chr7:98971820-98971650:2;206/chr15:1558:chr15:76639811-76639638:1;207/chr3:431:chr3:1
3591027-13590877:2;208/chr3:431:chr3:13591027-13590877:1;209/chr13:1184:chr13:109793440-109793267:2;
210/chr13:1184:chr13:109793440-109793267:1;211/chr7:3079:chr7:98971820-98971650:1;212/chrX:1761:chr
X:151142962-151142793:2;213/chrX:1761:chrX:151142962-151142793:1;214/chr6:852:chr6:28945366-289451
95:2;215/chr6:852:chr6:28945366-28945195:1;216/chr10:3652:chr10:126841036-126840898:1;217/chr11:105:c
hr11:640156-639983:2;218/chr11:105:chr11:640156-639983:1;219/chr6:890:chr6:29521568-29521403:2;220/ch
r6:890:chr6:29521568-29521403:1;221/chr18:994:chr18:55095196-55095066:2;222/chr18:994:chr18:55095196-
55095066:1;223/chr10:1053:chr10:30025825-30025652:2;224/chr10:1053:chr10:30025825-30025652:1;225/ch
r12:1757:chr12:95942874-95942730:2;226/chr12:1757:chr12:95942874-95942730:1;227/chr2:4233:chr2:17697
2445-176972287:2;228/chr2:4233:chr2:176972445-176972287:1;229/chr1:135:chr1:1118365-1118237:1;230/ch
r7:3586:chr7:129422136-129421964:2;231/chr7:3586:chr7:129422136-129421964:1;232/chr8:1893:chr8:65493
890-65493722:2;233/chr8:1893:chr8:65493890-65493722:1;234/chr20:555:chr20:21503414-21503264:2;235/ch
r20:555:chr20:21503414-21503264:1;236/chr22:2727:chr22:50738247-50738077:2;237/chr22:2727:chr22:5073
8247-50738077:1;238/chr22:2146:chr22:48885161-48884988:2;239/chr22:2146:chr22:48885161-48884988:1;2
40/chr17:2060:chr17:40822203-40822036:2;241/chr17:2060:chr17:40822203-40822036:1;242/chr7:4817:chr7:1
57478079-157477908:2;243/chr7:4817:chr7:157478079-157477908:1;244/chr22:294:chr22:20779881-2077971
0:2;245/chr22:294:chr22:20779881-20779710:1;246/chr10:2949:chr10:108924827-108924697:2;247/chr10:294
9:chr10:108924827-108924697:1;248/chr13:657:chr13:49795267-49795096:2;249/chr13:657:chr13:49795267-4
9795096:1;250/chr18:280:chr18:11752099-11751930:2;251/chr18:280:chr18:11752099-11751930:1;252/chr18:
1155:chr18:70534901-70534728:2;253/chr18:1155:chr18:70534901-70534728:1;254/chr6:458:chr6:12750199-1
2750024:2;255/chr5:1190:chr5:16179981-16179816:2;256/chr5:1190:chr5:16179981-16179816:1;257/chr6:458:
chr6:12750199-12750024:1;258/chr4:122:chr4:996466-996316:2;259/chr4:122:chr4:996466-996316:1;260/chr1
5:202:chr15:27018021-27017886:1;261/chr3:2134:chr3:123167367-123167219:2;262/chr3:2134:chr3:12316736
7-123167219:1;263/chr21:214:chr21:34442616-34442458:1;264/chr2:2631:chr2:99439224-99439049:2;265/chr
2:2631:chr2:99439224-99439049:1;266/chr18:92:chr18:5543725-5543553:2;267/chr18:92:chr18:5543725-5543
553:1;268/chr22:2208:chr22:48972303-48972136:1;269/chr8:2963:chr8:142428420-142428248:2;270/chr16:32
15:chr16:77468577-77468407:1;271/chr14:363:chr14:37136055-37135925:2;272/chr14:363:chr14:37136055-37
135925:1;273/chr4:759:chr4:5710116-5709949:2;274/chr4:759:chr4:5710116-5709949:1;275/chr14:343:chr14:3
7116474-37116302:2;276/chr14:343:chr14:37116474-37116302:1;277/chr2:1220:chr2:25438940-25438782:2;2
78/chr2:1220:chr2:25438940-25438782:1;279/chr6:138:chr6:1614837-1614673:2;280/chr6:138:chr6:1614837-1
614673:1;281/chr13:1053:chr13:100548262-100548120:2;282/chr13:1053:chr13:100548262-100548120:1;283/
chr10:4357:chr10:133796158-133795994:2;284/chr10:4357:chr10:133796158-133795994:1;285/chr8:2963:chr8:
142428420-142428248:1;286/chr10:2874:chr10:105344506-105344334:2;287/chr10:2874:chr10:105344506-10
5344334:1;288/chr4:470:chr4:2820416-2820245:2;289/chr4:470:chr4:2820416-2820245:1;290/chr1:224:chr1:14
75999-1475831:2;291/chr1:224:chr1:1475999-1475831:1;292/chr7:2651:chr7:71801536-71801361:1;293/chr2:1
346:chr2:27530576-27530436:2;294/chr2:1346:chr2:27530576-27530436:1;295/chrX:693:chrX:40005296-4000
5124:1;296/chr2:5206:chr2:236579734-236579578:2;297/chr2:5206:chr2:236579734-236579578:1;298/chr9:43
1:chr9:37002679-37002504:2;299/chr9:431:chr9:37002679-37002504:1;300/chr5:3644:chr5:178004124-178003
949:2;301/chr5:3644:chr5:178004124-178003949:1;302/chr10:2315:chr10:88127376-88127203:1;303/chr1:454
7:chr1:85513897-85513764:2;304/chr1:4547:chr1:85513897-85513764:1;305/chr7:1543:chr7:27213065-272129
00:2;306/chr7:1543:chr7:27213065-27212900:1;307/chrX:678:chrX:39949826-39949682:2;308/chrX:678:chrX:3
9949826-39949682:1;309/chr10:2761:chr10:102987102-102986950:2;310/chr10:2761:chr10:102987102-10298
6950:1;311/chr21:333:chr21:38081761-38081621:2;312/chr21:333:chr21:38081761-38081621:1;313/chr3:3332:
chr3:192233099-192232925:2;314/chr11:550:chr11:1995413-1995241:1;315/chr3:3332:chr3:192233099-19223
2925:1;316/chr14:2167:chr14:103396473-103396302:2;317/chr14:2167:chr14:103396473-103396302:1;318/chr
3:9:chr3:240109-239939:2;319/chr3:9:chr3:240109-239939:1;320/chr19:3833:chr19:46443576-46443407:2;321/
chr14:2758:chr14:105944625-105944473:2;322/chr14:2758:chr14:105944625-105944473:1;323/chr19:3833:chr
19:46443576-46443407:1;324/chr11:4166:chr11:119211505-119211357:2;325/chr11:4166:chr11:119211505-11
9211357:1;326/cg09639725:chr10:134901305-134901161:1;327/cg15825786:chr10:134901305-134901161:1;3
28/chr22:1894:chr22:46658866-46658705:2;329/chr22:1894:chr22:46658866-46658705:1;330/chr9:3185:chr9:1
38675144-138674988:2;331/chr9:3185:chr9:138675144-138674988:1;332/chr5:3298:chr5:174159305-1741591
30:2;333/chr5:3298:chr5:174159305-174159130:1;334/chr17:1937:chr17:38473317-38473192:1;335/chr9:1956:
Chr9:130516703-130516534:2;336/chr9:1956:chr9:130516703-130516534:1;337/chr11:1212:chr11:13031307-1
3031134:2;338/chr11:1212:chr11:13031307-13031134:1;339/chr17:270:chr17:1959038-1958866:2;340/chr17:2
70:chr17:1959038-1958866:1;341/chr5:3113:chr5:170289623-170289457:2;342/chr5:3113:chr5:170289623-170
289457:1;343/chr18:989:chr18:55019939-55019814:2;344/chr18:989:chr18:55019939-55019814:1;345/chr4:17
27:chr4:44450787-44450634:2;346/chr4:1727:chr4:44450787-44450634:1;347/chr3:2770:chr3:147128423-1471
28260:2;348/chr3:2770:chr3:147128423-147128260:1;349/chr9:1073:chr9:96715765-96715640:2;350/chr5:703:
chr5:2750656-2750482:2;351/chr5:703:chr5:2750656-2750482:1;352/chr4:2332:chr4:122302024-122301861:2;
353/chr9:2117:chr9:132382389-132382264:2;354/chr9:2117:chr9:132382389-132382264:1;355/chr2:605:chr2:5
837209-5837047:2;356/chr13:1196:chr13:110437232-110437061:2;357/chr13:1196:chr13:110437232-1104370
61:1;358/chr1:4393:chr1:65731623-65731457:2;359/chr1:4393:chr1:65731623-65731457:1;360/chr2:605:chr2:5
837209-5837047:1;361/chr19:1722:chr19:13617165-13616996:2;362/chr19:1722:chr19:13617165-13616996:1;
363/chr5:1725:chr5:72732819-72732644:2;364/chr5:1725:chr5:72732819-72732644:1;365/chr15:1477:chr15:75
248526-75248387:2;366/chr15:1477:chr15:75248526-75248387:1;367/chr13:518:chr13:37005646-37005519:2;
368/chr13:518:chr13:37005646-37005519:1;369/chr21:324:chr21:38077231-38077087:2;370/chr21:324:chr21:3
8077231-38077087:1;371/chr7:4817:chr7:157478460-157478320:2;372/chr7:4817:chr7:157478460-157478320:
1;373/chr1:1852:chr1:8277355-8277184:2;374/chr1:1852:chr1:8277355-8277184:1;375/chr8:3705:chr8:145107
103-145106943:2;376/chr8:3705:chr8:145107103-145106943:1;377/chr9:1073:chr9:96715765-96715640:1;378/
chr4:2332:chr4:122302024-122301861:1;379/chr13:1076:chr13:100641750-100641625:2;380/chr13:1076:chr13:
100641750-100641625:1;381/chr5:1664:chr5:66462501-66462352:1;382/chr10:1246:chr10:43600476-4360032
9:2;383/chr10:1246:chr10:43600476-43600329:1;384/chr1:5286:chr1:154475241-154475091:2;385/chr1:5286:c
hr1:154475241-154475091:1;386/chr6:2249:chr6:108439079-108438904:1;387/chr22:2148:chr22:48885967-48
885792:2;388/chr22:2148:chr22:48885967-48885792:1;389/chr12:1542:chr12:66123228-66123066:2;390/chr12:
1542:chr12:66123228-66123066:1;391/chr12:428:chr12:6665239-6665090:2;392/chr12:428:chr12:6665239-66
65090:1;393/chr15:1832:chr15:83953929-83953794:2;394/chr15:1832:chr15:83953929-83953794:1;395/chr11:
2301:chr11:63768349-63768184:2;396/chr11:2301:chr11:63768349-63768184:1;397/chr16:2445:chr16:543243
19-54324153:2;398/chr16:2445:chr16:54324319-54324153:1;399/chr17:2514:chr17:48195117-48194949:2;400/
chr17:2514:chr17:48195117-48194949:1;401/chr7:4816:chr7:157477728-157477553:2;402/chr7:4816:chr7:157
477728-157477553:1;403/chr22:1858:chr22:46480950-46480793:2;404/chr22:1858:chr22:46480950-46480793:
1;405/chr16:2477:chr16:54971887-54971737:2;406/chr16:2477:chr16:54971887-54971737:1;407/chr15:867:chr
15:53097714-53097557:2;408/chr15:867:chr15:53097714-53097557:1;409/chr5:2559:chr5:140871443-1408712
74:2;410/chr5:2559:chr5:140871443-140871274:1;411/chr1:4300:chr1:59042013-59041841:2;412/chr1:4300:ch
r1:59042013-59041841:1;413/chr12:1125:chr12:52215875-52215707:2;414/chr12:1125:chr12:52215875-52215
707:1;415/chr13:1184:chr13:109792924-109792792:2;416/chr13:1184:chr13:109792924-109792792:1;417/chr1
9:4004:chr19:48983973-48983798:2;418/chr19:4004:chr19:48983973-48983798:1;419/chr10:1246:chr10:43600
436-43600276:2;420/chr10:1246:chr10:43600436-43600276:1;421/chr1:4108:chr1:53308724-53308568:2;422/c
hr1:4108:chr1:53308724-53308568:1;423/chr3:1010:chr3:45187585-45187418:2;424/chr3:1010:chr3:45187585-
45187418:1;425/chr19:1717:chr19:13409497-13409361:2;426/chr19:1717:chr19:13409497-13409361:1;427/ch
r6:138:chr6:1614970-1614818:2;428/chr6:138:chr6:1614970-1614818:1;429/chr9:3751:chr9:140681330-14068
1159:1;430/chr17:2823:chr17:59529371-59529196:2;431/chr17:2823:chr17:59529371-59529196:1;432/chr15:2
091:chr15:91427666-91427519:2;433/chr15:2091:chr15:91427666-91427519:1;434/chr7:405:chr7:1276323-127
6152:2;435/chr7:405:chr7:1276323-1276152:1;436/chr19:4075:chr19:49575723-49575570:2;437/chr19:4075:ch
r19:49575723-49575570:1;438/chr8:42:chr8:688810-688660:2;439/chr8:42:chr8:688810-688660:1;440/chr3:10
10:chr3:45188219-45188047:1;441/chr12:3097:chr12:130648449-130648282:2;442/chr12:3097:chr12:1306484
49-130648282:1;443/chr19:2639:chr19:30866221-30866047:2;444/chr19:2639:chr19:30866221-30866047:1;44
5/chr3:2172:chr3:124860818-124860646:2;446/chr3:2172:chr3:124860818-124860646:1;447/chr5:1864:chr5:80
256326-80256157:2;448/chr5:1864:chr5:80256326-80256157:1;449/chr19:1696:chr19:13210149-13209979:2;4
50/chr19:1696:chr19:13210149-13209979:1;451/chr8:791:chr8:15397803-15397628:2;452/chr8:791:chr8:15397
803-15397628:1;453/chr10:2698:chr10:102587280-102587146:2;454/chr10:2698:chr10:102587280-102587146:
1;455/chr16:679:chr16:2287070-2286919:2;456/chr7:1497:chr7:27155280-27155120:2;457/chr7:1536:chr7:272
05908-27205733:2;458/chr7:1536:chr7:27205908-27205733:1;459/chr16:679:chr16:2287070-2286919:1;460/ch
r7:1497:chr7:27155280-27155120:1;461/chr1:604:chr1:2461567-2461430:2;462/chr1:604:chr1:2461567-24614
30:1;463/chr18:1153:chr18:70534486-70534312:2;464/chr18:1153:chr18:70534486-70534312:1;465/chr3:1611:
chr3:65342234-65342081:2;466/chr3:1611:chr3:65342234-65342081:1;467/chr11:1079:chr11:6651650-665148
4:2;468/chr11:1079:chr11:6651650-6651484:1;469/chr1:5882:chr1:180202481-180202309:2;470/chr1:5882:chr
1:180202481-180202309:1;471/chr1:254:chr1:1567020-1566850:2;472/chr1:254:chr1:1567020-1566850:1;473/
cg14455998:chr7:133811940-133811768:1;474/chr14:847:chr14:69095416-69095241:2;475/chr7:3500:chr7:12
7671519-127671351:2;476/chr7:3500:chr7:127671519-127671351:1;477/chr14:847:chr14:69095416-69095241:
1;478/chr20:2719:chr20:61703616-61703454:2;479/chr20:2719:chr20:61703616-61703454:1;480/chr8:1701:chr
8:54163459-54163292:2;481/chr8:1701:chr8:54163459-54163292:1;482/chr17:2281:chr17:44897033-44896861:
2;483/chr5:1664:chr5:66462741-66462568:2;484/chr5:1664:chr5:66462741-66462568:1;485/cg09360501:chr1
9:22019096-22018924:1;486/chr5:1434:chr5:42952324-42952151:2;487/chr17:2799:chr17:59474326-59474160:
2;488/chr17:2799:chr17:59474326-59474160:1;489/chr4:1664:chr4:40859350-40859177:2;490/chr4:1664:chr4:
40859350-40859177:1;491/chr12:307:chr12:4919217-4919078:2;492/chr12:307:chr12:4919217-4919078:1;493/
chr9:1226:chr9:100616810-100616659:2;494/chr9:1226:chr9:100616810-100616659:1;495/chr5:1434:chr5:429
52324-42952151:1;496/chr2:525:chr2:3751410-3751244:2;497/chr2:525:chr2:3751410-3751244:1;498/chr4:127
2:chr4:8863888-8863725:2;499/chr4:1272:chr4:8863888-8863725:1;500/chr17:4139:chr17:77788965-77788791:
2;501/chr17:4139:chr17:77788965-77788791:1;502/chr15:1553:chr15:76635583-76635420:2;503/chr15:1553:c
hr15:76635583-76635420:1;504/chr5:1172:chr5:15500436-15500269:2;505/chr5:1172:chr5:15500436-1550026
9:1;506/chr17:2281:chr17:44897033-44896861:1;507/chr1:59:chr1:969452-969286:2;508/chr1:59:chr1:969452-
969286:1;509/chr2:2750:chr2:105459768-105459599:2;510/chr2:2750:chr2:105459768-105459599:1;511/chr7:
1858:chr7:37487561-37487390:2;512/chr7:1858:chr7:37487561-37487390:1;513/chr4:2610:chr4:155665124-15
5664949:2;514/chr4:2610:chr4:155665124-155664949:1;515/chr19:551:chr19:2251307-2251141:2;516/chr19:5
51:chr19:2251307-2251141:1;517/chr5:3119:chr5:170736998-170736824:2;518/chr5:3119:chr5:170736998-170
736824:1;519/chr8:2236:chr8:99952450-99952290:2;520/chr8:2236:chr8:99952450-99952290:1;521/chr2:2631:
chr2:99439453-99439305:2;522/chr2:2631:chr2:99439453-99439305:1;523/chr1:4838:chr1:110754955-110754
788:2;524/chr1:4838:chr1:110754955-110754788:1;525/chr12:1258:chr12:54071277-54071152:2;526/chr12:12
58:chr12:54071277-54071152:1;527/chr16:2511:chr16:55358398-55358271:2;528/chr16:2511:chr16:55358398-
55358271:1;529/chr21:1265:chr21:47062845-47062720:2;530/chr21:1265:chr21:47062845-47062720:1;531/chr
6:2182:chr6:101851019-101850850:2;532/chr6:2182:chr6:101851019-101850850:1;533/chr15:523:chr15:37172
741-37172571:2;534/chr15:523:chr15:37172741-37172571:1;535/chr6:1985:chr6:85484520-85484393:2;536/ch
r6:1985:chr6:85484520-85484393:1;537/chr20:1322:chr20:43727361-43727195:2;538/chr20:1322:chr20:43727
361-43727195:1;539/chr8:941:chr8:21647516-21647385:2;540/chr8:941:chr8:21647516-21647385:1;541/chr18:
765:chr18:44337902-44337746:2;542/chr18:765:chr18:44337902-44337746:1;543/chr7:562:chr7:1626134-1625
961:2;544/chr7:562:chr7:1626134-1625961:1;545/chr13:1468:chr13:112727908-112727782:2;546/chr13:1468:c
hr13:112727908-112727782:1;547/chr11:2460:chr11:64815221-64815088:2;548/chr11:2460:chr11:64815221-6
4815088:1;549/chr12:1282:chr12:54346064-54345892:2;550/chr12:1282:chr12:54346064-54345892:1;551/cg1
0471437:chr16:28074607-28074451:2;552/cg10471437:chr16:28074607-28074451:1;553/chr5:2392:chr5:1352
66327-135266164:2;554/chr5:2392:chr5:135266327-135266164:1;555/cg03257172:chr1:200010306-20001013
7:1;556/chr7:1552:chr7:27225329-27225188:2;557/chr8:1403:chr8:38757738-38757583:1;558/cg13432708:chr
4:4388612-4388440:2;559/cg13432708:chr4:43886124388440:1;560/chr6:789:chr6:27725301-27725157:2;561/
chr12:1318:chr12:54427265-54427095:2;562/chr2:597:chr2:5832275-5832109:2;563/chr15:1198:chr15:681194
62-68119300:2;564/chr15:1198:chr15:68119462-68119300:1;565/chr12:1318:chr12:54427265-54427095:1;566/
chr2:597:chr2:5832275-5832109:1;567/chr9:1081:chr9:96721556-96721405:2;568/chr9:1081:chr9:96721556-96
721405:1;569/chr1:3851:chr1:44873440-44873315:2;570/chr1:3851:chr1:44873440-44873315:1;571/chr1:5180:
chr1:151810817-151810653:2;572/chr1:5180:chr1:151810817-151810653:1;573/chr2:1181:chr2:24232980-242
32828:2;574/chr2:1181:chr2:24232980-24232828:1;575/chr2:4268:chr2:177016548-177016375:2;576/chr2:426
8:chr2:177016548-177016375:1;577/chr4:5:chr4:107599-107466:2;578/chr4:5:chr4:107599-107466:1;579/chr17:
275:chr17:1960855-1960723:2;580/chr17:275:chr17:1960855-1960723:1;581/chr15:219:chr15:27216564-2721
6427:2;582/chr15:219:chr15:27216564-27216427:1;583/chr13:1687:chr13:113699673-113699501:2;584/chr19:
830:chr19:3786359-3786184:2;585/chr15:260:chr15:28342343-28342201:2;586/chr15:260:chr15:28342343-283
42201:1;587/chr7:3315:chr7:103630408-103630249:2;588/chr7:3315:chr7:103630408-103630249:1;589/chr5:3
993:chr5:180486753-180486590:2;590/chr5:3993:chr5:180486753-180486590:1;591/chr22:2727:chr22:507385
60-50738390:2;592/chr22:2727:chr22:50738560-50738390:1;593/chr8:1876:chr8:65282188-65282052:2;594/ch
r8:1876:chr8:65282188-65282052:1;595/chr5:1434:chr5:42952230-42952059:2;596/chr5:1434:chr5:42952230-
42952059:1;597/chr11:389:chr11:1578414-1578271:2;598/chr17:537:chr17:4804204-4804043:2;599/chr9:402:c
hr9:36739888-36739756:2;600/chr9:402:chr9:36739888-36739756:1;601/chr3:823:chr3:37494343-37494176:2;
602/chr3:823:chr3:37494343-37494176:1;603/chr10:3172:chr10:118894210-118894054:2;604/chr10:3172:chr1
0:118894210-118894054:1;605/chr12:1084:chr12:50297975-50297804:2;606/chr12:1084:chr12:50297975-5029
7804:1;607/chr17:2535:chr17:48546150-48546021:2;608/chr17:2535:chr17:48546150-48546021:1;609/chr18:1
06:chr18:5891142-5891001:2;610/chr18:106:chr18:5891142-5891001:1;611/chr16:224:chr16:1024589-1024418:
2;612/chr19:3997:chr19:48946382-48946227:2;613/chr19:3997:chr19:48946382-48946227:1;614/chr17:1003:c
hr17:11144079-11143912:2;615/chr17:1003:chr17:11144079-11143912:1;616/chr9:1226:chr9:100617150-1006
17003:2;617/chr9:1226:chr9:100617150-100617003:1;618/chr22:2764:chr22:50987475-50987303:2;619/chr10:
3240:chr10:119494662-119494502:2;620/chr10:3240:chr10:119494662-119494502:1;621/chr7:1376:chr7:2324
5923-23245776:2;622/chr15:2015:chr15:89952790-89952620:1;623/chr6:2182:chr6:101850697-101850525:1;6
24/chr17:1855:chr17:36666963-36666805:2;625/chr5:3993:chr5:180486893-180486726:2;626/chr5:3993:chr5:1
80486893-180486726:1;627/chr8:1408:chr8:38965351-38965177:2;628/chr8:1408:chr8:38965351-38965177:1;
629/chr10:4270:chr10:133110301-133110163:2;630/chr10:4270:chr10:133110301-133110163:1;631/chr1:6544:
chr1:214153480-214153323:2;632/chr1:6544:chr1:214153480-214153323:1;633/chr4:2508:chr4:147576330-14
7576168:2;634/chr4:2508:chr4:147576330-147576168:1;635/chr7:4724:chr7:157072115-157071948:2;636/chr7:
4724:chr7:157072115-157071948:1;637/chr12:3094:chr12:130646863-130646696:2;638/chr12:3094:chr12:130
646863-130646696:1;639/chr17:1855:chr17:36666963-36666805:1;640/chr19:1717:chr19:13409607-13409433:
2;641/chr19:1717:chr19:13409607-13409433:1;642/chr6:1972:chr6:84419152-84418977:2;643/chr6:1972:chr6:
84419152-84418977:1;644/chr4:1747:chr4:48485548-48485417:2;645/chr4:1747:chr4:48485548-48485417:1;6
46/chr12:1462:chr12:58131732-58131563:2;647/chr12:1462:chr12:58131732-58131563:1;648/chr16:224:chr16:
1024589-1024418:1;649/chr19:368:chr19:1469199-1469031:2;650/chr19:368:chr19:1469199-1469031:1;651/ch
r2:257:chr2:1747345-1747170:2;652/chr2:257:chr2:1747345-1747170:1;653/chr22:2764:chr22:50987475-5098
7303:1;654/chr7:4109:chr7:150655660-150655489:2;655/chr7:4109:chr7:150655660-150655489:1;656/chr7:13
76:chr7:23245923-23245776:1;657/chr4:637:chr4:3769495-3769348:2;658/chr4:637:chr4:3769495-3769348:1;6
59/chr1:4827:chr1:110613313-110613165:2;660/chr1:4827:chr1:110613313-110613165:1;661/chr13:714:chr13:
53421515-53421349:2;662/chr13:714:chr13:53421515-53421349:1;663/chr1:4913:chr1:113287108-113286975:
2;664/chr1:4913:chr1:113287108-113286975:1;665/chr12:1757:chr12:95942707-95942574:2;666/chr12:1757:c
hr12:95942707-95942574:1;667/chr16:4191:chr16:88701237-88701108:2;668/chr16:4191:chr16:88701237-887
01108:1;669/chr12:2984:chr12:129338120-129337948:2;670/chr12:2984:chr12:129338120-129337948:1;671/c
hr2:1560:chr2:42720600-42720440:2;672/chr18:1046:chr18:56941329-56941156:2;673/chr18:1046:chr18:5694
1329-56941156:1;674/chr2:1560:chr2:4272060042720440:1;675/chr2:259:chr2:1748325-1748159:2;676/chr17:
2349:chr17:46604643-46604494:2;677/chr17:2349:chr17:46604643-46604494:1;678/chr17:1004:chr17:111449
43-11144805:2;679/chr17:1004:chr17:11144943-11144805:1;680/chr2:259:chr2:1748325-1748159:1;681/chr15:
2253:chr15:95388449-95388283:2;682/chr15:2253:chr15:95388449-95388283:1;683/chr7:1552:chr7:27225329-
27225188:1;684/chr13:1458:chr13:112722066-112721911:2;685/chr13:1458:chr13:112722066-112721911:1;68
6/chr18:91:chr18:5543242-5543067:2;687/chr18:91:chr18:5543242-5543067:1;688/chr16:74:chr16:610817-610
648:2;689/chr16:74:chr16:610817-610648:1;690/chr6:86:chr6:1312460-1312285:2;691/chr6:86:chr6:1312460-1
312285:1;692/chr6:789:chr6:27725301-27725157:1;693/chr10:4942:chr10:135193098-135192933:2;694/chr10:
4942:chr10:135193098-135192933:1;695/chr11:2620:chr11:66885578-66885411:2;696/chr11:2620:chr11:6688
5578-66885411:1;697/chr16:4191:chr16:88700886-88700720:2;698/chr16:4191:chr16:88700886-88700720:1;6
99/chr1:59:chr1:969070-968901:2;700/chr1:59:chr1:969070-968901:1;701/chr1:4547:chr1:85514129-85513954:
2;702/chr1:4547:chr1:85514129-85513954:1;703/chr1:1408:chr1:4715328-4715192:2;704/chr1:1408:chr1:4715
328-4715192:1;705/chr12:428:chr12:6664597-6664426:2;706/chr12:428:chr12:6664597-6664426:1;707/chr16:2
389:chr16:51186360-51186234:2;708/chr16:2389:chr16:51186360-51186234:1;709/chr1:1408:chr1:4714681-47
14541:2;710/chr1:1408:chr1:4714681-4714541:1;711/chr3:3517:chr3:196255871-196255701:2;712/chr3:3517:c
hr3:196255871-196255701:1;713/chr13:1687:chr13:113699673-113699501:1;714/chr4:1272:chr4:8863594-886
3462:2;715/chr4:1272:chr4:8863594-8863462:1;716/chr7:1307:chr7:19156981-19156840:2;717/chr7:1307:chr7:
19156981-19156840:1;718/chr15:839:chr15:52405049-52404876:2;719/chr15:839:chr15:52405049-52404876:1;
720/chr11:320:chr11:1411918-1411750:2;721/chr11:320:chr11:1411918-1411750:1;722/chr19:830:chr19:3786
359-3786184:1;723/chr1:1408:chr1:4714986-4714816:2;724/chr1:1408:chr1:4714986-4714816:1;725/chr19:169
6:chr19:13210215-13210088:2;726/chr19:1696:chr19:13210215-13210088:1;727/chr13:85:chr13:20875922-208
75760:2;728/chr13:85:chr13:20875922-20875760:1;729/chr6:99:chr6:1384254-1384099:2;730/chr6:99:chr6:138
4254-1384099:1;731/chr5:1826:chr5:77806363-77806192:2;732/chr5:1826:chr5:77806363-77806192:1;733/chr
10:3445:chr10:124896916-124896777:2;734/chr10:3445:chr10:124896916-124896777:1;735/chr11:389:chr11:1
578414-1578271:1;736/chr17:537:chr17:4804204-4804043:1;737/chr3:5:chr3:239100-238936:2;738/chr3:5:chr3:
239100-238936:1;739/chr10:2308:chr10:88123111-88122936:2;740/chr10:2308:chr10:88123111-88122936:1;7
41/chr21:333:chr21:38081666-38081528:2;742/chr21:333:chr21:38081666-38081528:1;743/chr1:4640:chr1:913
01915-91301785:2;744/chr1:4640:chr1:91301915-91301785:1;745/chr5:1789:chr5:76373422-76373260:2;746/c
hr5:1789:chr5:76373422-76373260:1;747/chr7:4820:chr7:157481855-157481686:2;748/chr7:4820:chr7:157481
855-157481686:1;749/chr2:4262:chr2:176994812-176994654:2;750/chr2:4262:chr2:176994812-176994654:1;7
51/chr3:2753:chr3:147111581-147111456:2;752/chr3:2753:chr3:147111581-147111456:1;753/chr1:4838:chr1:1
10754666-110754532:2;754/chr1:4838:chr1:110754666-110754532:1;755/chr5:2560:chr5:140872047-1408719
11:2;756/chr5:2560:chr5:140872047-140871911:1;757/chr16:2882:chr16:67428666-67428530:2;758/chr16:288
2:chr16:67428666-67428530:1;759/chr6:1322:chr6:35992413-35992248:2;760/chr6:1322:chr6:35992413-35992
248:1;761/chr17:1316:chr17:19628181-19628024:2;762/chr17:1316:chr17:19628181-19628024:1;763/chr3:823:
chr3:37494467-37494341:2;764/chr3:823:chr3:37494467-37494341:1;765/chr11:4686:chr11:130067781-13006
7608:2;766/chr9:60:chr9:1051743-1051568:2;767/chr9:60:chr9:1051743-1051568:1;768/chr11:4686:chr11:1300
67781-130067608:1;769/chr20:1878:chr20:55965360-55965187:2;770/chr20:1878:chr20:55965360-55965187:1;
771/chr5:2559:chr5:140871367-140871209:2;772/chr5:2559:chr5:140871367-140871209:1;773/chr11:296:chr1
1:1358676-1358546:2;774/chr11:296:chr11:1358676-1358546:1;775/chr10:2401:chr10:94449587-94449453:2;7
76/chr10:2401:chr10:94449587-94449453:1;777/chr4:123:chr4:997013-996870:2;778/chr4:123:chr4:997013-99
6870:1;779/chr9:1080:chr9:96720974-96720799:2;780/chr9:1080:chr9:96720974-96720799:1;781/chr10:2315:c
hr10:88127439-88127279:2;782/chr10:2315:chr10:88127439-88127279:1;783/chr18:989:chr18:55019875-5501
9712:2;784/chr21:1253:chr21:47010310-47010150:1;785/chr7:3763:chr7:139167913-139167769:2;786/chr7:37
63:chr7:139167913-139167769:1;787/chr7:1543:chr7:27213116-27212965:2;788/chr7:1543:chr7:27213116-272
12965:1;789/chr2:4772:chr2:220313573-220313429:2;790/chr2:4772:chr2:220313573-220313429:1;791/chr22:
161:chr22:19711159-19710984:2;792/chr22:161:chr22:19711159-19710984:1;793/chr6:1788:chr6:54711761-54
711632:2;794/chr6:1788:chr6:54711761-54711632:1;795/chr19:3621:chr19:42703810-42703641:1;796/chr10:31
80:chr10:118899850-118899718:2;797/chr10:3180:chr10:118899850-118899718:1;798/chr13:1458:chr13:112
721977-112721835:2;799/chr13:1458:chr13:112721977-112721835:1;800/chr7:562:chr7:1626359-1626234:2;8
01/chr7:562:chr7:1626359-1626234:1;802/chr17:273:chr17:1959936-1959811:2;803/chr17:273:chr17:1959936-
1959811:1;804/chr10:1049:chr10:30024665-30024512:2;805/chr10:1049:chr10:30024665-30024512:1;806/chr1
0:2024:chr10:77156975-77156850:2;807/chr10:2024:chr10:77156975-77156850:1;808/chr12:1090:chr12:50355
164-50355023:2;809/chr1:7109:chr1:234040945-234040813:2;810/chr1:7109:chr1:234040945-234040813:1;81
1/chr12:1090:chr12:50355164-50355023:1;812/chr15:839:chr15:52404728-52404567:2;813/chr15:839:chr15:52
404728-52404567:1;814/chr11:1079:chr11:6651865-6651739:2;815/chr11:1079:chr11:6651865-6651739:1;816/
chr22:1246:chr22:40045741-40045566:1;817/chr13:1456:chr13:112721241-112721116:2;818/chr13:1456:chr13:
112721241-112721116:1;819/chr19:3321:chr19:39086994-39086826:2;820/chr19:3321:chr19:39086994-39086
826:1;821/chr18:1609:chr18:76739213-76739058:2;822/chr7:1568:chr7:27260502-27260331:2;823/chr7:1568:c
hr7:27260502-27260331:1;824/chr18:1609:chr18:76739213-76739058:1;825/chr8:1959:chr8:70946981-709468
17:2;826/chr10:2740:chr10:102900355-102900186:2;827/chr11:1427:chr11:20385333-20385195:2;828/chr11:1
427:chr11:20385333-20385195:1;829/chr10:2740:chr10:102900355-102900186:1;830/chr11:2620:chr11:66885
940-66885789:2;831/chr11:2620:chr11:66885940-66885789:1;832/chrX:666:chrX:39864870-39864699:2;833/c
hrX:666:chrX:39864870-39864699:1;834/chr8:1959:chr8:70946981-70946817:1;835/chrX:678:chrX:39949547-3
9949372:2;836/chrX:678:chrX:39949547-39949372:1;837/chr1:691:chr1:2777505-2777378:2;838/chr1:691:chr1:
2777505-2777378:1;839/chr10:846:chr10:21789327-21789154:2;840/chr10:846:chr10:21789327-21789154:1;8
41/chr1:7412:chr1:248020840-248020670:2;842/chr1:7412:chr1:248020840-248020670:1;843/chr2:4122:chr2:1
72948274-172948103:2;844/chr2:4122:chr2:172948274-172948103:1;845/chr19:4862:chr19:58038848-580386
73:2;846/chr19:4862:chr19:58038848-58038673:1;847/chr18:1256:chr18:73628544-73628399:2;848/chr18:125
6:chr18:73628544-73628399:1;849/chr10:4898:chr10:135052157-135051986:2;850/chr10:4898:chr10:1350521
57-135051986:1;851/chr20:732:chr20:25129250-25129125:2;852/chr20:732:chr20:25129250-25129125:1;853/c
hr5:2392:chr5:135266648-135266505:2;854/chr5:2392:chr5:135266648-135266505:1;855/chr13:1184:chr13:10
9793673-109793539:2;856/chr13:1184:chr13:109793673-109793539:1;857/chr10:4732:chr10:134844916-1348
44745:2;858/chr10:4732:chr10:134844916-134844745:1;859/chr9:3509:chr9:139741633-139741490:2;860/chr9:
3509:chr9:139741633-139741490:1;861/chr8:1701:chr8:54163588-54163457:2;862/chr8:1701:chr8:54163588-
54163457:1;863/chr1:3957:chr1:47904941-47904774:2;864/chr1:3957:chr1:47904941-47904774:1;865/chr19:4
216:chr19:50861948-50861773:2;866/chr19:4216:chr19:50861948-50861773:1;867/chr19:4796:chr19:5698901
0-56988868:2;868/chr19:4796:chr19:56989010-56988868:1;869/chr4:202:chr4:1398790-1398615:2;870/chr4:20
2:chr4:1398790-1398615:1;871/cg15344220:chr11:8040582-8040407:2;872/cg15344220:chr11:8040582-80404
07:1;873/chr11:5:chr11:280536-280381:2;874/chr11:5:chr11:280536-280381:1;875/chr19:3621:chr19:42703748-
42703577:2;876/chr19:3621:chr19:42703748-42703577:1;877/chr4:1750:chr4:48493326-48493161:2;878/chr4:
1750:chr4:48493326-48493161:1;879/chr15:275:chr15:29131520-29131346:2;880/chr15:275:chr15:29131520-2
9131346:1;881/chr7:1533:chr7:27205115-27204944:2;882/chr7:1533:chr7:27205115-27204944:1;883/chr16:11
2:chr16:745332-745166:2;884/chr5:3916:chr5:180018219-180018091:2;885/chr5:3916:chr5:180018219-180018
091:1;886/chr16:112:chr16:745332-745166:1;887/chr20:134:chr20:2673091-2672939:2;888/chr20:134:chr20:26
73091-2672939:1;889/chr10:906:chr10:23481389-23481221:1;890/chr9:402:chr9:36739816-36739691:2;891/ch
r9:402:chr9:36739816-36739691:1;892/chr5:2489:chr5:139228292-139228144:2;893/chr5:2489:chr5:13922829
2-139228144:1;894/chr18:503:chr18:24130644-24130486:2;895/chr18:503:chr18:24130644-24130486:1;896/ch
r8:263:chr8:1444022-1443847:2;897/chr8:263:chr8:1444022-1443847:1;898/chr17:4148:chr17:77814401-7781
4276:2;899/chr17:4148:chr17:77814401-77814276:1;900/chr19:2488:chr19:29284801-29284634:2;901/chr19:2
488:chr19:29284801-29284634:1;902/chr3:2394:chr3:128274390-128274215:2;903/chr3:2394:chr3:128274390-
128274215:1;904/chr20:2210:chr20:59827833-59827677:2;905/chr20:2210:chr20:59827833-59827677:1;906/c
hr14:2327:chr14:104622286-104622118:2;907/chr14:2327:chr14:104622286-104622118:1;908/chr4:759:chr4:5
710066-5709903:2;909/chr4:759:chr4:5710066-5709903:1;910/chr2:741:chr2:8816855-8816691:2;911/chr11:22
24:chr11:62212270-62212144:1;912/chr6:1985:chr6:85484648-85484498:2;913/chr6:1985:chr6:85484648-8548
4498:1;914/chr2:741:chr2:8816855-8816691:1;915/chr3:2629:chr3:138658377-138658219:2;916/chr3:2629:chr
3:138658377-138658219:1;917/cg07710481:chr13:88325462-88325302:1;918/chr20:136:chr20:2674782-26746
44:2;919/chr18:776:chr18:44774139-44773967:2;920/chr18:776:chr18:44774139-44773967:1;921/chr20:136:ch
r20:2674782-2674644:1;922/chr8:2051:chr8:80804255-80804087:2;923/chr8:2051:chr8:80804255-80804087:1;
924/chrX:503:chrX:21676440-21676273:2;925/chrX:503:chrX:21676440-21676273:1;926/chr16:1124:chr16:910
7288-9107124:1;927/chr20:620:chr20:23029611-23029456:2;928/chr2:4233:chr2:176972745-176972591:2;929/
chr2:4233:chr2:176972745-176972591:1;930/cg15778745:chr13:88324623-88324465:2;931/eg15778745:chr13:
88324623-88324465:1;932/chr13:100:chr13:21296008-21295833:2;933/chr10:848:chr10:21799349-21799198:
2;934/chr10:848:chr10:21799349-21799198:1;935/chr19:367:chr19:1467934-1467793:2;936/chr19:367:chr19:1
467934-1467793:1;937/chr2:38:chr2:468520-468356:2;938/chr19:4654:chr19:55598756-55598631:2;939/chr20:
1833:chr20:55500445-55500279:2;940/chr7:1908:chr7:39649532-39649358:2;941/chr7:1908:chr7:39649532-39
649358:1;942/chr4:2703:chr4:172734459-172734312:2;943/chr17:2176:chr17:43045392-43045228:1;944/chr18:
1804:chr18:77389578-77389404:2;945/chr8:13:chr8:494891-494765:2;946/chr8:13:chr8:494891-494765:1;947/
chr17:1814:chr17:35300168-35299994:2;948/chr14:363:chr14:37136352-37136182:2;949/chr19:4654:chr19:55
598756-55598631:1;950/chr21:102:chr21:28338043-28337869:2;951/chr21:102:chr21:28338043-28337869:1;9
52/chr1:971:chr1:3155348-3155173:2;953/chr1:971:chr1:3155348-3155173:1;954/chr2:4127:chr2:172954828-1
72954655:1;955/chr11:4458:chr11:125036547-125036399:2;956/chr11:1427:chr11:20385438-20385265:2;957/
chr6:1985:chr6:85484335-85484208:2;958/chr12:1112:chr12:51718045-51717920:2;959/chr12:1112:chr12:517
18045-51717920:1;960/chr9:3255:chr9:139024824-139024679:2;961/chr9:3255:chr9:139024824-139024679:1;
962/chr5:1949:chr5:92940228-92940090:1;963/chr6:1567:chr6:42072403-42072258:2;964/chr6:1567:chr6:4207
2403-42072258:1;965/chr12:3095:chr12:130647408-130647283:2;966/chr12:3095:chr12:130647408-13064728
3:1;967/chr4:1288:chr4:8895327-8895159:2;968/chr22:1515:chr22:43807550-43807412:2;969/chr22:1515:chr2
2:43807550-43807412:1;970/chr18:1150:chr18:70209552-70209377:2;971/chr18:1150:chr18:70209552-702093
77:1;972/chr12:1169:chr12:52627367-52627205:2;973/chr11:105:chr11:640276-640122:2;974/chr11:105:chr11:
640276-640122:1;975/chr17:2778:chr17:58227629-58227469:2;976/chr1:1254:chr1:3567760-3567621:1;977/ch
r18:763:chr18:44337035-44336860:2;978/chr5:3347:chr5:175224373-175224225:2;979/chr5:3347:chr5:175224
373-175224225:1;980/chr5:1719:chr5:72678238-72678084:2;981/chr5:1719:chr5:72678238-72678084:1;982/ch
r14:343:chr14:37116730-37116572:2;983/chr14:2041:chr14:101925384-101925236:2;984/chr14:2041:chr14:10
1925384-101925236:1;985/chr16:234:chr16:1035465-1035307:2;986/chr16:234:chr16:1035465-1035307:1;987/
chr7:4544:chr7:155596079-155595930:2;988/chr7:4544:chr7:155596079-155595930:1;989/chr16:1569:chr16:2
2825758-22825609:2;990/chr16:1569:chr16:22825758-22825609:1;991/chr13:368:chr13:28674805-28674668:2;
992/chr13:368:chr13:28674805-28674668:1;993/chr18:1613:chr18:76740511-76740349:2;994/chr18:1613:chr1
8:76740511-76740349:1;995/chr22:1515:chr22:43807805-43807680:2;996/chr22:1515:chr22:43807805-438076
80:1;997/chr7:1858:chr7:37487837-37487671:1;998/chr7:282:chr7:751906-751765:2;999/chr7:282:chr7:751906-
751765:1;1000/chr17:2778:chr17:58227629-58227469:1;1001/chr5:838:chr5:3600857-3600685:2;1002/chr5:83
8:chr5:3600857-3600685:1;1003/chr18:763:chr18:44337035-44336860:1;1004/chr14:343:chr14:37116730-3711
6572:1;1005/chr19:567:chr19:2291026-2290868:2;1006/chr19:567:chr19:2291026-2290868:1;1007/chr5:2324:c
hr5:134363943-134363791:2;1008/chr5:2324:chr5:134363943-134363791:1;1009/chr14:1435:chr14:93154393-
93154237:2;1010/chr14:1435:chr14:93154393-93154237:1;1011/chr11:389:chr11:1578154-1578018:2;1012/chr
11:389:chr11:1578154-1578018:1;1013/chr11:4458:chr11:125036547-125036399:1;1014/chr12:2890:chr12:127
211367-127211206:2;1015/chr12:2890:chr12:127211367-127211206:1;1016/chr11:1427:chr11:20385438-2038
5265:1;1017/chr6:1985:chr6:85484335-85484208:1;1018/chr17:965:chr17:10102074-10101912:2;1019/chr17:9
65:chr17:10102074-10101912:1;1020/chr7:4817:chr7:157478309-157478184:2;1021/chr7:4817:chr7:15747830
9-157478184:1;1022/chr2:3107:chr2:115919030-115918868:2;1023/chr2:3107:chr2:115919030-115918868:1;1
024/chr14:586:chr14:57275916-57275777:2;1025/chr14:586:chr14:57275916-57275777:1;1026/chr19:4075:chr
19:49576064-49575897:2;1027/chr19:4075:chr19:49576064-49575897:1;1028/chr4:1288:chr4:8895327-889515
9:1;1029/chr17:4511:chr17:79615343-79615170:2;1030/chr17:4511:chr17:79615343-79615170:1;1031/chr5:83
0:chr5:3596349-3596188:2;1032/chr5:830:chr5:3596349-3596188:1;1033/chr10:983:chr10:28034954-28034829:
2;1034/chr10:983:chr10:28034954-28034829:1;1035/chr3:1614:chr3:65535276-65535103:2;1036/chr3:1614:ch
r3:65535276-65535103:1;1037/chr14:2099:chr14:102172394-102172221:2;1038/chr14:2099:chr14:102172394-
102172221:1;1039/chr12:1169:chr12:52627367-52627205:1;1040/chr6:1746:chr6:50818431-50818259:2;1041/
chr6:1746:chr6:50818431-50818259:1;1042/chr19:1946:chr19:16436639-16436493:2;1043/chr19:1946:chr19:1
6436639-16436493:1;1044/chr12:1140:chr12:52301350-52301213:2;1045/chr12:1140:chr12:52301350-523012
13:1;1046/chr12:2587:chr12:122016613-122016440:2;1047/chr12:2587:chr12:122016613-122016440:1;1048/c
hr3:333:chr3:13115130-13115000:2;1049/chr3:333:chr3:13115130-13115000:1;1050/chr3:2382:chr3:12821090
1-128210726:2;1051/chr3:2382:chr3:128210901-128210726:1;1052/chr3:2384:chr3:128211778-128211603:1;1
053/chr19:3997:chr19:48946258-48946107:2;1054/chr19:3997:chr19:48946258-48946107:1;1055/chr1:3196:ch
r1:29102083-29101911:2;1056/chr1:3196:chr1:29102083-29101911:1;1057/chr18:1835:chr18:77548423-77548
256:2;1058/chr18:1835:chr18:77548423-77548256:1;1059/chr11:2133:chr11:61277255-61277115:2;1060/chr11:
2133:chr11:61277255-61277115:1;1061/chr6:380:chr6:10382089-10381934:2;1062/chr6:380:chr6:10382089-1
0381934:1;1063/chr4:1290:chr4:8895839-8895689:2;1064/chr18:765:chr18:44338199-44338045:2;1065/chr18:
765:chr18:44338199-44338045:1;1066/chr10:1049:chr10:30024548-30024417:2;1067/chr10:1049:chr10:30024
548-30024417:1;1068/cg08521987:chr10:119000955-119000814:1;1069/chr7:873:chr7:3341384-3341224:2;10
70/chr7:873:chr7:3341384-3341224:1;1071/chr2:3708:chr2:131721444-131721299:2;1072/chr2:3708:chr2:1317
21444-131721299:1;1073/chr4:1290:chr4:8895839-8895689:1;1074/chr4:2602:chr4:155411561-155411399:2;1
075/chr4:2602:chr4:155411561-155411399:1;1076/chr2:4224:chr2:176957850-176957713:2;1077/chr2:4224:ch
r2:176957850-176957713:1;1078/chr9:2143:chr9:132651075-132650918:1;1079/chr11:2420:chr11:64428600-6
4428433:2;1080/chr11:2420:chr11:64428600-64428433:1;1081/chr10:2138:chr10:81002139-81001995:2;1082/
chr10:2138:chr10:81002139-81001995:1;1083/chr6:98:chr6:1383867-1383692:2;1084/chr6:98:chr6:1383867-13
83692:1;1085/chr17:4139:chr17:77789183-77789053:2;1086/chr17:4139:chr17:77789183-77789053:1;1087/chr
17:658:chr17:6899650-6899489:2;1088/chr17:658:chr17:6899650-6899489:1;1089/chr6:596:chr6:19837625-19
837468:2;1090/chr6:596:chr6:19837625-19837468:1;1091/chr2:118:chr2:946865-946706:2;1092/chr2:118:chr2:
946865-946706:1;1093/chr1:1852:chr1:8277192-8277048:2;1094/chr1:1852:chr1:8277192-8277048:1;1095/chr
9:1287:chr9:102590791-102590666:2;1096/chr9:1287:chr9:102590791-102590666:1;1097/chr8:1107:chr8:2477
1208-24771045:2;1098/chr8:1107:chr8:24771208-24771045:1;1099/chr19:1696:chr19:13210537-13210412:2;1
100/chr19:1696:chr19:13210537-13210412:1;1101/chr22:295:chr22:20780522-20780352:2;1102/chr22:295:chr
22:20780522-20780352:1;1103/chr1:604:chr1:2461653-2461488:2;1104/chr1:604:chr1:2461653-2461488:1;110
5/chr17:1558:chr17:28088493-28088329:2;1106/chr17:1558:chr17:28088493-28088329:1;1107/chr18:1609:chr
18:76739292-76739118:2;1108/chr18:1609:chr18:76739292-76739118:1;1109/chr14:2727:chr14:105715006-10
5714843:2;1110/chr14:2727:chr14:105715006-105714843:1;1111/chr14:627:chr14:59932154-59931989:2;1112/
chr14:627:chr14:59932154-59931989:1;1113/chr16:1299:chr16:12995863-12995736:2;1114/chr16:1299:chr16:
12995863-12995736:1;1115/chr1:7249:chr1:240256720-240256595:2;1116/chr1:7249:chr1:240256720-240256
595:1;1117/chr2:1926:chr2:63287004-63286866:2;1118/chr2:1926:chr2:63287004-63286866:1;1119/chr14:369:
chr14:37667305-37667159:2;1120/chr14:369:chr14:37667305-37667159:1;1121/chr7:1307:chr7:19157141-191
56967:2;1122/chr7:1307:chr7:19157141-19156967:1;1123/chr20:620:chr20:23029611-23029456:1;1124/chr8:8
09:chr8:17271163-17271004:2;1125/chr15:219:chr15:27216798-27216666:2;1126/chr15:219:chr15:27216798-2
7216666:1;1127/chr13:100:chr13:21296008-21295833:1;1128/chr6:1985:chr6:85484190-85484020:2;1129/chr6:
596:chr6:19837558-19837402:2;1130/chr2:38:chr2:468520-468356:1;1131/chr6:596:chr6:19837558-19837402:
1;1132/chr20:1833:chr20:55500445-55500279:1;1133/chr4:2703:chr4:172734459-172734312:1;1134/chr18:180
4:chr18:77389578-77389404:1;1135/chr13:100:chr13:21296134-21296008:2;1136/chr13:100:chr13:21296134-2
1296008:1;1137/chr17:1814:chr17:35300168-35299994:1;1138/chr14:379:chr14:38057530-38057362:2;1139/c
hr14:379:chr14:38057530-38057362:1;1140/chr14:363:chr14:37136352-37136182:1;1141/chr8:809:chr8:17271
163-17271004:1;1142/chr6:1985:chr6:85484190-85484020:1;1143/chr1:3539:chr1:37499734-37499586:2;1144/
chr1:3539:chr1:37499734-37499586:1;1145/chr22:2146:chr22:48885256-48885117:2;1146/chr22:2146:chr22:4
8885256-48885117:1;1147/chr18:77:chr18:4455520-4455345:2;1148/chr18:77:chr18:4455520-4455345:1;1149/
chr16:375:chr16:1203334-1203167:2;1150/chr16:375:chr16:1203334-1203167:1;1151/chr14:343:chr14:371165
76-37116434:2;1152/chr14:343:chr14:37116576-37116434:1;1153/chr1:4547:chr1:85514236-85514067:2;1154/
chr1:4547:chr1:85514236-85514067:1;1155/chr17:1003:chr17:11144402-11144227:2;1156/chr17:1003:chr17:1
1144402-11144227:1;1157/chr7:3136:chr7:100318355-100318219:2;1158/chr7:3136:chr7:100318355-1003182
19:1;1159/chr7:4153:chr7:150812030-150811874:2;1160/chr7:4153:chr7:150812030-150811874:1;1161/chr11:
2301:chr11:63768057-63767885:2;1162/chr11:2301:chr11:63768057-63767885:1;1163/chr2:4268:chr2:177016
631-177016505:2;1164/chr2:4268:chr2:177016631-177016505:1;1165/chr9:343:chr9:34809888-34809723:2;11
66/chr12:428:chr12:6664740-6664574:2;1167/chr12:428:chr12:6664740-6664574:1;1168/chr11:1094:chr11:769
5117-7694967:2;1169/chr11:1094:chr11:7695117-7694967:1;1170/chr9:343:chr9:34809888-34809723:1;1171/c
hr16:3215:chr16:77468687-77468523:2;1172/chr16:3215:chr16:77468687-77468523:1;1173/cg18456523:chr5:
54516951-54516804:1;1174/chr5:1679:chr5:68711451-68711276:2;1175/chr5:1679:chr5:68711451-68711276:1;
1176/chr1:5181:chr1:151811374-151811199:2;1177/chr1:5181:chr1:151811374-151811199:1;1178/chr16:2389:
chr16:51186569-51186438:2;1179/chr16:2389:chr16:51186569-51186438:1;1180/chr15:2091:chr15:91427593-
91427434:2;1181/chr15:2091:chr15:91427593-91427434:1;1182/chr4:1405:chr4:13545813-13545648:2;1183/c
hr2:5231:chr2:237072614-237072463:2;1184/chr2:5231:chr2:237072614-237072463:1;1185/chr4:1405:chr4:13
545813-13545648:1;1186/chr21:1182:chr21:46848176-46848010:2;1187/chr21:1182:chr21:46848176-4684801
0:1;1188/chr11:1511:chr11:31827114-31826942:2;1189/chr11:1511:chr11:31827114-31826942:1;1190/chr17:2
060:chr17:40822498-40822329:2;1191/chr17:2060:chr17:40822498-40822329:1;1192/chr22:2727:chr22:50737
784-50737647:1;1193/chr16:1299:chr16:12996019-12995848:2;1194/chr16:1299:chr16:12996019-12995848:1;
1195/chr18:1125:chr18:63418264-63418098:2;1196/chr18:1125:chr18:63418264-63418098:1;1197/chr5:2198:c
hr5:127874673-127874515:2;1198/chr5:2198:chr5:127874673-127874515:1;1199/chr1:253:chr1:1566309-1566
164:2;1200/chr1:253:chr1:1566309-1566164:1;1201/chr19:4939:chr19:58868430-58868255:2;1202/chr19:4939:
chr19:58868430-58868255:1;1203/chr2:5244:chr2:237081601-237081427:1;1204/chr19:368:chr19:1469580-14
69455:2;1205/chr19:368:chr19:1469580-1469455:1;1206/chr8:738:chr8:11560058-11559891:2;1207/chr8:738:c
hr8:11560058-11559891:1;1208/chr10:4942:chr10:135193158-135193014:2;1209/chr10:4942:chr10:135193158-
135193014:1;1210/chr1:359:chr1:2005635-2005485:1;1211/chr1:2371:chr1:16085571-16085408:2;1212/chr1:2
371:chr1:16085571-16085408:1;1213/chr2:2432:chr2:89065078-89064903:1;1214/cg14649650:chr10:1393402
9-13933861:2;1215/cg14649650:chr10:13934029-13933861:1;1216/chr1:222:chr1:1475176-1475024:2;1217/ch
r1:222:chr1:1475176-1475024:1;1218/chr20:924:chr20:32254757-32254591:2;1219/chr7:3502:chr7:127672794-
127672628:2;1220/chr7:3502:chr7:127672794-127672628:1;1221/chr2:5420:chr2:239756031-239755883:2;12
22/chr2:5420:chr2:239756031-239755883:1;1223/chr22:1367:chr22:42679893-42679731:2;1224/chr22:1367:ch
r22:42679893-42679731:1;1225/chr20:924:chr20:32254757-32254591:1;1226/chr5:91:chr5:528913-528766:2;1
227/chr5:91:chr5:528913-528766:1;1228/chr13:1053:chr13:100548459-100548313:2;1229/chr13:1053:chr13:10
0548459-100548313:1;1230/chr7:4818:chr7:157478660-157478508:2;1231/chr7:4818:chr7:157478660-157478
508:1;1232/chr14:931:chr14:72399532-72399383:2;1233/chr2:2852:chr2:107503201-107503061:2;1234/chr2:2
852:chr2:107503201-107503061:1;1235/chr14:931:chr14:72399532-72399383:1;1236/chr7:1486:chr7:2714188
9-27141719:2;1237/chr7:1486:chr7:27141889-27141719:1;1238/chr2:1263:chr2:26407815-26407679:2;1239/ch
r2:1263:chr2:26407815-26407679:1;1240/chr10:983:chr10:28034760-28034588:2;1241/chr10:983:chr10:28034
760-28034588:1;1242/chr20:924:chr20:32255023-32254875:2;1243/chr20:924:chr20:32255023-32254875:1;12
44/chr1:4371:chr1:63789664-63789496:2;1245/chr1:4371:chr1:63789664-63789496:1;1246/chr21:324:chr21:38
077303-38077138:2;1247/chr21:324:chr21:38077303-38077138:1;1248/chr1:3370:chr1:32827642-32827468:2;
1249/chr1:3370:chr1:32827642-32827468:1;1250/chr8:2241:chr8:99960969-99960794:2;1251/chr8:2241:chr8:9
9960969-99960794:1;1252/chr1:4177:chr1:54203574-54203399:2;1253/chr1:4177:chr1:54203574-54203399:1;
1254/chr8:1959:chr8:70947045-70946889:2;1255/chr8:1959:chr8:70947045-70946889:1;1256/chr1:2205:chr1:1
2123709-12123550:2;1257/chr1:2205:chr1:12123709-12123550:1;1258/chr15:1553:chr15:76635433-76635267:
2;1259/chr15:1553:chr15:76635433-76635267:1;1260/chr1:831:chr1:2987148-2987022:2;1261/chr1:831:chr1:2
987148-2987022:1;1262/chr20:136:chr20:2674616-2674449:2;1263/chr20:136:chr20:2674616-2674449:1;1264/
chr13:1782:chr13:114523993-114523818:2;1265/chr13:1782:chr13:114523993-114523818:1;1266/chr9:343:chr
9:34810106-34809981:2;1267/chr9:343:chr9:34810106-34809981:1;1268/chr8:1006:chr8:22409029-22408887:
2;1269/chr8:1006:chr8:22409029-22408887:1;1270/chr9:3136:chr9:138606582-138606424:2;1271/chr9:3136:c
hr9:138606582-138606424:1;1272/chr20:2210:chr20:59827027-59826882:2;1273/chr20:2210:chr20:59827027-
59826882:1;1274/chr4:15:chr4:330482-330342:2;1275/chr4:1403:chr4:13544202-13544031:2;1276/chr19:562:c
hr19:2282730-2282564:1;1277/chr4:15:chr4:330482-330342:1;1278/chr4:1403:chr4:13544202-13544031:1;127
9/chr18:1611:chr18:76740192-76740051:2;1280/chr18:1611:chr18:76740192-76740051:1;1281/chr7:727:chr7:2
539980-2539830:1;1282/chr11:141:chr11:849963-849796:2;1283/chr11:141:chr11:849963-849796:1;1284/cg16
791424:chr14:27068157-27067985:1;1285/chr1:3150:chr1:27687341-27687204:2;1286/chr1:3150:chr1:276873
41-27687204:1;1287/chr4:1270:chr4:8862937-8862789:2;1288/chr4:1270:chr4:8862937-8862789:1;1289/chr10:
3516:chr10:125751261-125751128:2;1290/chr10:3516:chr10:125751261-125751128:1;1291/chr15:219:chr15:2
7216334-27216209:2;1292/chr15:219:chr15:27216334-27216209:1;1293/chr17:3421:chr17:73074038-7307386
4:1;1294/chr6:93:chr6:1314245-1314076:2;1295/chr6:93:chr6:1314245-1314076:1;1296/chr11:2506:chr11:6540
8451-65408278:2;1297/chr11:2506:chr11:65408451-65408278:1;1298/chr2:1220:chr2:25439097-25438923:2;1
299/chr2:1220:chr2:25439097-25438923:1;1300/chr10:4269:chr10:133109705-133109558:2;1301/chr10:4269:c
hr10:133109705-133109558:1;1302/chr15:2019:chr15:89955002-89954827:2;1303/chr15:2019:chr15:89955002-
89954827:1;1304/chr1:4364:chr1:63786122-63785973:1;1305/chr10:1133:chr10:35929284-35929117:2;1306/c
hr10:1133:chr10:35929284-35929117:1;1307/chr4:2647:chr4:164265081-164264908:2;1308/chr4:2647:chr4:16
4265081-164264908:1;1309/chr1:4827:chr1:110613115-110612942:2;1310/chr1:4827:chr1:110613115-110612
942:1;1311/chr9:1374:chr9:112081565-112081412:2;1312/chr9:1374:chr9:112081565-112081412:1;1313/chr5:
3116:chr5:170735801-170735626:2;1314/chr6:3116:chr5:170735801-170735626:1;1315/chr14:411:chr14:3872
4877-38724714:2;1316/chr14:411:chr14:38724877-38724714:1;1317/chr12:1452:chr12:58021724-58021565:2;
1318/chr12:1452:chr12:58021724-58021565:1;1319/chr2:257:chr2:1747069-1746939:2;1320/chr2:257:chr2:174
7069-1746939:1;1321/chr13:401:chr13:29394356-29394206:2;1322/chr13:401:chr13:29394356-29394206:1;13
23/chr22:1288:chr22:40391389-40391226:2;1324/chr22:1288:chr22:40391389-40391226:1;1325/chr15:983:chr
15:60297160-60296999:2;1326/chr15:983:chr15:60297160-60296999:1;1327/chr18:1054:chr18:57364519-5736
4384:2;1328/chr18:1054:chr18:57364519-57364384:1;1329/chr19:4862:chr19:58038676-58038509:2;1330/chr1
9:4862:chr19:58038676-58038509:1;1331/chr13:1108:chr13:102569198-102569062:2;1332/chr13:1108:chr13:1
02569198-102569062:1;1333/chr12:3052:chr12:130389080-130388955:2;1334/chr12:3052:chr12:130389080-1
30388955:1;1335/chr5:1718:chr5:72677453-72677280:2;1336/chr5:1718:chr5:72677453-72677280:1;1337/chr5:
829:chr5:3595934-3595761:2;1338/chr5:829:chr5:3595934-3595761:1;1339/cg21039708:chr14:57278810-572
78636:1;1340/chr4:1702:chr4:42153735-42153562:2;1341/chr4:1702:chr4:42153735-42153562:1;1342/chr5:22
1:chr5:1059274-1059119:2;1343/chr5:221:chr5:1059274-1059119:1;1344/chr2:3075:chr2:114035222-11403504
7:1;1345/chr19:267:chr19:1169102-1168933:2;1346/chr19:267:chr19:1169102-1168933:1;1347/chr10:2700:chr
10:102587682-102587520:2;1348/cg08167706:chr7:134144258-134144100:1;1349/chr10:3652:chr10:1268409
67-126840797:2;1350/chr20:925:chr20:32255745-32255575:2;1351/chr20:925:chr20:32255745-32255575:1;13
52/chr19:3840:chr19:46518724-46518577:2;1353/chr19:3840:chr19:46518724-46518577:1;1354/chr2:1519:chr
2:39187360-39187185:2;1355/chr2:1519:chr2:39187360-39187185:1;1356/chr18:75:chr18:4454785-4454611:2;
1357/chr18:75:chr18:4454785-4454611:1;1358/chr12:1542:chr12:66123260-66123121:2;1359/chr12:1542:chr1
2:66123260-66123121:1;1360/chr9:629:chr9:79635570-79635395:2;1361/chr9:629:chr9:79635570-79635395:1;
1362/chr2:2063:chr2:71116441-71116267:2;1363/chr2:2063:chr2:71116441-71116267:1;1364/chr12:2984:chr1
2:129338639-129338472:2;1365/chr12:2984:chr12:129338639-129338472:1;1366/chr14:363:chr14:37136284-3
7136114:2;1367/chr14:363:chr14:37136284-37136114:1;1368/chr13:519:chr13:37006155-37005994:2;1369/chr
13:519:chr13:37006155-37005994:1;1370/chr22:2774:chr22:51042732-51042561:2;1371/chr22:2774:chr22:510
42732-51042561:1;1372/chr15:275:chr15:29131090-29130948:2;1373/chr15:275:chr15:29131090-29130948:1;
1374/chr12:1090:chr12:50355297-50355159:2;1375/chr12:1090:chr12:50355297-50355159:1;1376/chr6:2190:c
hr6:105389224-105389096:2;1377/chr6:2190:chr6:105389224-105389096:1;1378/chr7:4153:chr7:150812343-1
50812215:2;1379/chr7:4153:chr7:150812343-150812215:1;1380/chr20:525:chr20:21377259-21377094:2;1381/
chr20:525:chr20:21377259-21377094:1;1382/chr10:2700:chr10:102587682-102587520:1;1383/chr10:3652:chr1
0:126840967-126840797:1;1384/chr2:2233:chr2:74875337-74875170:2;1385/chr2:2233:chr2:74875337-748751
70:1;1386/chr1:3371:chr1:32828021-32827850:2;1387/chr1:3371:chr1:32828021-32827850:1;1388/chr16:241:c
hr16:1040802-1040629:1;1389/chr3:2897:chr3:157821435-157821261:2;1390/chr3:2897:chr3:157821435-1578
21261:1;1391/chr5:2522:chr5:140306278-140306136:2;1392/chr5:2522:chr5:140306278-140306136:1;1393/chr
22:2146:chr22:48885627-48885471:2;1394/chr22:2146:chr22:48885627-48885471:1;1395/chr2:4268:chr2:1770
16786-177016616:2;1396/chr17:1522:chr17:27038943-27038773:2;1397/chr17:1522:chr17:27038943-2703877
3:1;1398/chr9:1427:chr9:116226070-116225907:2;1399/chr9:1427:chr9:116226070-116225907:1;1400/chr3:13
8:chr3:8810227-8810102:2;1401/chr3:138:chr3:8810227-8810102:1;1402/chr2:4268:chr2:177016786-17701661
6:1;1403/chr8:1788:chr8:57358189-57358050:1;1404/chr12:3095:chr12:130647184-130647019:2;1405/chr12:3
095:chr12:130647184-130647019:1;1406/chr7:4786:chr7:157406328-157406165:2;1407/chr7:4786:chr7:15740
6328-157406165:1;1408/chr19:4874:chr19:58239078-58238903:1;1409/chr9:4:chr9:113639-113513:2;1410/chr
9:4:chr9:113639-113513:1;1411/chr1:7210:chr1:237206324-237206168:2;1412/chr1:7210:chr1:237206324-237
206168:1;1413/chr19:3923:chr19:47910442-47910317:2;1414/chr12:1112:chr12:51717830-51717655:2;1415/c
hr19:3278:chr19:38886821-38886696:2;1416/chr19:3278:chr19:38886821-38886696:1;1417/chr19:3923:chr19:
47910504-47910369:2;1418/chr19:3923:chr19:47910504-47910369:1;1419/chr19:2540:chr19:30017174-30017
010:2;1420/chr19:2540:chr19:30017174-30017010:1;1421/chr17:4170:chr17:77916930-77916765:2;1422/chr17:
4170:chr17:77916930-77916765:1;1423/chr7:4539:chr7:155580021-155579858:2;1424/chr7:4539:chr7:155580
021-155579858:1;1425/chr7:873:chr7:3341305-3341174:2;1426/chr7:873:chr7:3341305-3341174:1;1427/chr4:1
23:chr4:997253-997081:2;1428/chr4:123:chr4:997253-997081:1;1429/chr1:5180:chr1:151810731-151810557:2;
1430/chr1:5180:chr1:151810731-151810557:1;1431/chr10:2874:chr10:105344571-105344411:2;1432/chr10:28
74:chr10:105344571-105344411:1;1433/chr7:2589:chr7:70597385-70597237:2;1434/chr7:2589:chr7:70597385-
70597237:1;1435/chr6:144:chr6:1620548-1620376:2;1436/chr6:144:chr6:1620548-1620376:1;1437/chr12:1523:
chr12:65218668-65218543:2;1438/chr12:1523:chr12:65218668-65218543:1;1439/chr22:1858:chr22:46481122-
46480950:2;1440/chr22:1858:chr22:46481122-46480950:1;1441/chr14:1137:chr14:77492702-77492527:2;1442/
chr14:1137:chr14:77492702-77492527:1;1443/chr15:207:chr15:27112981-27112832:2;1444/chr10:801:chr10:1
7496201-17496073:2;1445/chr10:801:chr10:17496201-17496073:1;1446/chr10:2140:chr10:81003231-8100308
7:2;1447/chr10:2140:chr10:81003231-81003087:1;1448/chr22:369:chr22:22863167-22862997:2;1449/chr22:36
9:chr22:22863167-22862997:1;1450/chr5:3695:chr5:178421685-178421544:2;1451/chr5:3695:chr5:178421685-
178421544:1;1452/chr1:1196:chr1:3464917-3464745:2;1453/chr1:1196:chr1:3464917-3464745:1;1454/chr19:5
66:chr19:2290579-2290446:2;1455/chr19:566:chr19:2290579-2290446:1;1456/chr15:264:chr15:28352965-2835
2820:2;1457/chr11:3710:chr11:100998989-100998854:2;1458/chr11:3710:chr11:100998989-100998854:1;1459/
chr8:354:chr8:1950282-1950148:2;1460/chr8:354:chr8:1950282-1950148:1;1461/chr10:467:chr10:7450319-74
50156:2;1462/chr10:467:chr10:7450319-7450156:1;1463/chr13:356:chr13:28550209-28550072:2;1464/chr13:3
56:chr13:28550209-28550072:1;1465/chr22:2791:chr22:51135947-51135790:2;1466/chr22:2791:chr22:511359
47-51135790:1;1467/chr2:1921:chr2:63283036-63282867:2;1468/chr2:1921:chr2:63283036-63282867:1;1469/c
hr19:2488:chr19:29284706-29284548:2;1470/chr19:2488:chr19:29284706-29284548:1;1471/chr15:207:chr15:2
7112981-27112832:1;1472/chr22:1921:chr22:46932592-46932420:2;1473/chr22:1921:chr22:46932592-469324
20:1;1474/chr12:2942:chr12:128752178-128752024:2;1475/chr7:4818:chr7:157478747-157478586:2;1476/chr7:
4818:chr7:157478747-157478586:1;1477/chr12:2942:chr12:128752178-128752024:1;1478/chr17:279:chr17:19
62444-1962288:2;1479/chr17:279:chr17:1962444-1962288:1;1480/cg17332326:chr19:37997586-37997412:1;1
481/chr3:1095:chr3:47050951-47050803:2;1482/chr1:3916:chr1:46951560-46951399:2;1483/chr15:1558:chr15:
76639518-76639393:2;1484/chr8:40:chr8:688182-688010:2;1485/chr8:40:chr8:688182-688010:1;1486/chr14:36
9:chr14:37667357-37667229:2;1487/chr14:369:chr14:37667357-37667229:1;1488/chr17:534:chr17:4802644-48
02489:2;1489/chr17:534:chr17:4802644-4802489:1;1490/chr1:250:chr1:1565027-1564889:2;1491/chr1:250:chr
1:1565027-1564889:1;1492/chr7:2696:chr7:72848212-72848057:2;1493/chr7:2696:chr7:72848212-72848057:1;
1494/chr4:207:chr4:1400795-1400625:2;1495/chr18:105:chr18:5890801-5890652:2;1496/chr18:105:chr18:5890
801-5890652:1;1497/chr15:2011:chr15:89950895-89950720:2;1498/chr13:877:chr13:79183624-79183462:2;14
99/chr13:877:chr13:79183624-79183462:1;1500/chr11:4458:chr11:125036677-125036542:2;1501/chr11:4458:c
hr11:125036677-125036542:1;1502/chr22:1852:chr22:46475957-46475784:2;1503/chr19:367:chr19:1468394-1
468234:2;1504/chr19:367:chr19:1468394-1468234:1;1505/chr2:1346:chr2:27530799-27530635:2;1506/chr2:13
46:chr2:27530799-27530635:1;1507/chr9:3509:chr9:139741503-139741332:2;1508/chr9:3509:chr9:139741503-
139741332:1;1509/chr22:2703:chr22:50623575-50623404:2;1510/chr22:2703:chr22:50623575-50623404:1;151
1/chr4:2032:chr4:81124127-81123963:2;1512/chr4:2032:chr4:81124127-81123963:1;1513/chr19:4708:chr19:55
997910-55997742:2;1514/chr6:256:chr6:5086536-5086395:2;1515/chr6:256:chr6:5086536-5086395:1;1516/chr
4:1481:chr4:20256633-20256503:2;1517/chr4:1481:chr4:20256633-20256503:1;1518/chr4:773:chr4:5894233-5
894060:2;1519/chr4:773:chr4:5894233-5894060:1;1520/cg17872757:chr11:128564260-128564128:2;1521/chr1:
7209:chr1:237206009-237205864:2;1522/chr1:7209:chr1:237206009-237205864:1;1523/chr12:307:chr12:4918
937-4918794:2;1524/chr12:307:chr12:4918937-4918794:1;1525/chr1:3709:chr1:41327704-41327547:2;1526/ch
r1:3709:chr1:41327704-41327547:1;1527/chr17:2375:chr17:46711323-46711149:2;1528/chr2:3997:chr2:16228
0699-162280561:2;1529/chr2:3997:chr2:162280699-162280561:1;1530/chr22:2526:chr22:49969753-49969615:
2;1531/chr22:2526:chr22:4996975349969615:1;1532/chr1:1196:chr1:3464847-3464695:1;1533/chr10:524:chr1
0:8098176-8098032:2;1534/chr10:524:chr10:8098176-8098032:1;1535/chr8:1742:chr8:55379931-55379778:2;1
536/chr8:1742:chr8:55379931-55379778:1;1537/chr6:2198:chr5:127874548-127874405:2;1538/chr5:2198:chr5:
127874548-127874405:1;1539/chr20:2210:chr20:59828073-59827941:2;1540/chr20:2210:chr20:59828073-598
27941:1;1541/chr9:135:chr9:8857697-8857539:2;1542/chr9:135:chr9:8857697-8857539:1;1543/chr10:2911:chr
10:106401341-106401173:2;1544/chr10:2911:chr10:106401341-106401173:1;1545/chr14:341:chr14:37053269-
37053129:1;1546/chr4:1277:chr4:8873708-8873557:2;1547/chr4:1277:chr4:8873708-8873557:1;1548/cg06072
021:chr11:128564147-128563972:2;1549/cg06072021:chr11:128564147-128563972:1;1550/chr13:1184:chr13:
109793537-109793408:2;1551/chr13:1184:chr13:109793537-109793408:1;1552/chr3:1502:chr3:58572844-585
72702:1;1553/chr8:3701:chr8:145104374-145104222:2;1554/chr1:359:chr1:2005416-2005254:2;1555/chr1:359:
chr1:2005416-2005254:1;1556/chr11:4686:chr11:130067833-130067661:2;1557/chr11:4686:chr11:130067833-
130067661:1;1558/chr3:138:chr3:8809999-8809856:2;1559/chr3:138:chr3:8809999-8809856:1;1560/chr1:5181:
chr1:151811440-151811314:2;1561/chr1:5181:chr1:151811440-151811314:1;1562/chr22:294:chr22:20779652-
20779494:2;1563/chr22:294:chr22:20779652-20779494:1;1564/chr14:1137:chr14:77492434-77492309:2;1565/
chr14:1137:chr14:77492434-77492309:1;1566/chr19:367:chr19:1467540-1467377:2;1567/chr19:367:chr19:146
7540-1467377:1;1568/chr8:1878:chr8:65282739-65282588:2;1569/chr8:1878:chr8:65282739-65282588:1;1570/
chr18:280:chr18:11751686-11751551:2;1571/chr18:280:chr18:11751686-11751551:1;1572/chr22:2676:chr22:5
0496975-50496837:2;1573/chr22:2676:chr22:50496975-50496837:1;1574/chr19:211:chr19:1042311-1042139:2;
1575/chr19:211:chr19:1042311-1042139:1;1576/chr18:1613:chr18:76740750-76740605:2;1577/chr18:1613:chr
18:76740750-76740605:1;1578/chr14:323:chr14:36987207-36987032:2;1579/chr16:1569:chr16:22825918-2282
5749:2;1580/chr16:1569:chr16:22825918-22825749:1;1581/chr14:323:chr14:36987207-36987032:1;1582/chr1:
1594:chr1:6126343-6126173:2;1583/chr8:3759:chr8:145698738-145698597:2;1584/chr8:3759:chr8:145698738-
145698597:1;1585/chr3:2134:chr3:123167247-123167100:2;1586/chr11:98:chr11:627188-627038:2;1587/chr14:
2038:chr14:101923887-101923723:2;1588/chr3:2134:chr3:123167247-123167100:1;1589/chr11:98:chr11:6271
88-627038:1;1590/chr14:2038:chr14:101923887-101923723:1;1591/chr12:1463:chr12:58131815-58131671:2;1
592/chr12:1463:chr12:58131815-58131671:1;1593/chr12:1125:chr12:52215491-52215337:2;1594/chr12:1125:c
hr12:52215491-52215337:1;1595/chr10:2138:chr10:81002332-81002207:1;1596/chr8:39:chr8:687729-687560:
2;1597/chr8:39:chr8:687729-687560:1;1598/chr1:1594:chr1:6126343-6126173:1;1599/chr1:4827:chr1:1106132
20-110613071:2;1600/chr1:4827:chr1:110613220-110613071:1;1601/chr8:2236:chr8:99952394-99952226:2;16
02/chr8:2236:chr8:99952394-99952226:1;1603/chr8:941:chr8:21647736-21647578:2;1604/chr8:941:chr8:21647
736-21647578:1;1605/chr16:1569:chr16:22825629-22825465:2;1606/chr16:1569:chr16:22825629-22825465:1;
1607/chr1:1254:chr1:3567687-3567562:2;1608/chr1:1254:chr1:3567687-3567562:1;1609/chr19:1690:chr19:132
07491-13207357:2;1610/chr19:1690:chr19:13207491-13207357:1;1611/chr12:1858:chr12:104610140-1046100
05:2;1612/chr12:1858:chr12:104610140-104610005:1;1613/chr3:2641:chr3:138679606-138679477:2;1614/chr3:
2641:chr3:138679606-138679477:1;1615/chr22:2774:chr22:51043024-51042853:2;1616/chr22:2774:chr22:510
43024-51042853:1;1617/chr10:2140:chr10:81003133-81002965:2;1618/chr10:2140:chr10:81003133-81002965:
1;1619/chr12:1090:chr12:50355556-50355398:2;1620/chr12:1090:chr12:50355556-50355398:1;1621/chr1:3150:
chr1:27687217-27687073:2;1622/chr1:3150:chr1:27687217-27687073:1;1623/chr3:5:chr3:238958-238785:2;16
24/chr3:5:chr3:238958-238785:1;1625/chr22:163:chr22:19711965-19711838:2;1626/chr22:163:chr22:19711965-
19711838:1;1627/chr9:1705:chr9:126775225-126775052:2;1628/chr9:1705:chr9:126775225-126775052:1;162
9/chr12:1860:chr12:104697497-104697323:2;1630/chr12:1860:chr12:104697497-104697323:1;1631/chr2:3996:
chr2:162280049-162279883:2;1632/chr2:3996:chr2:162280049-162279883:1;1633/chr12:157:chr12:2800894-2
800749:2;1634/chr12:157:chr12:2800894-2800749:1;1635/chr8:3701:chr8:145104374-145104222:1;1636/chr19:
368:chr19:1469284-1469111:2;1637/chr19:368:chr19:1469284-1469111:1;1638/chr9:3297:chr9:139094963-13
9094789:2;1639/chr9:3297:chr9:139094963-139094789:1;1640/chr20:55:chr20:826065-825900:2;1641/chr20:5
5:chr20:826065-825900:1;1642/chr8:2284:chr8:102506385-102506236:2;1643/chr8:2284:chr8:102506385-1025
06236:1;1644/chr10:2911:chr10:106401064-106400899:2;1645/chr10:2911:chr10:106401064-106400899:1;164
6/chr14:2727:chr14:105714945-105714771:2;1647/chr14:2727:chr14:105714945-105714771:1;1648/chr15:250
1:chr15:101513727-101513562:2;1649/chr15:2501:chr15:101513727-101513562:1;1650/chr19:367:chr19:1467
811-1467685:2;1651/chr19:367:chr19:1467811-1467685:1;1652/chr16:4179:chr16:88601120-88600956:2;1653/
chr16:4179:chr16:88601120-88600956:1;1654/chr1:1594:chr1:6126259-6126084:2;1655/chr1:1594:chr1:61262
59-6126084:1;1656/chr6:1788:chr6:54711496-54711328:2;1657/chr8:650:chr8:10587987-10587836:2;1658/chr
8:650:chr8:10587987-10587836:1;1659/chr18:776:chr18:44774027-44773887:2;1660/chr18:776:chr18:4477402
7-44773887:1;1661/chr6:1788:chr6:54711496-54711328:1;1662/chr19:1180:chr19:6745018-6744847:1;1663/ch
r1:4908:chr1:113051739-113051598:2;1664/chr1:4908:chr1:113051739-113051598:1;1665/chr5:1789:chr5:763
73664-76373502:2;1666/chr5:1789:chr5:76373664-76373502:1;1667/chr4:2602:chr4:155411852-155411694:2;
1668/chr4:2602:chr4:155411852-155411694:1;1669/chr4:24:chr4:569110-568938:2;1670/chr4:24:chr4:569110-
568938:1;1671/chr5:3347:chr5:175224022-175223871:2;1672/chr5:3347:chr5:175224022-175223871:1;1673/c
hr7:3502:chr7:127672698-127672546:2;1674/chr7:3502:chr7:127672698-127672546:1;1675/chr2:5038:chr2:23
3285412-233285282:2;1676/chr2:5038:chr2:233285412-233285282:1;1677/chr1:59:chr1:969126-968986:2;167
8/chr1:59:chr1:969126-968986:1;1679/chr12:1112:chr12:51718149-51718011:2;1680/chr16:224:chr16:1024644-
1024472:1;1681/chrX:784:chrX:47433584-47433422:2;1682/chr4:109:chr4:967754-967593:2;1683/chr4:109:ch
r4:967754-967593:1;1684/cg06993118:chr5:127874080-127873917:2;1685/chr11:2667:chr11:67291071-67290
897:1;1686/cg06993118:chr5:127874080-127873917:1;1687/chr1:4054:chr1:50889152-50888992:2;1688/chr1:
4054:chr1:50889152-50888992:1;1689/chrX:784:chrX:47433584-47433422:1;1690/chr11:550:chr11:1995499-1
995325:2;1691/chr11:550:chr11:1995499-1995325:1;1692/chr10:2670:chr10:102483906-102483732:1;1693/chr
5:3858:chr5:179243985-179243848:2;1694/chr5:3858:chr5:179243985-179243848:1;1695/chr12:1112:chr12:51
718149-51718011:1;1696/chr19:3157:chr19:37096458-37096329:2;1697/chr19:3157:chr19:37096458-3709632
9:1;1698/chr15:1556:chr15:76638706-76638531:2;1699/chr15:1556:chr15:76638706-76638531:1;1700/chr1:48
27:chr1:110613043-110612888:2;1701/chr1:4827:chr1:110613043-110612888:1;1702/chr19:3923:chr19:47910
442-47910317:1;1703/chr12:1112:chr12:51717830-51717655:1;1704/chr1:4913:chr1:113287046-113286876:2;
1705/chr1:4913:chr1:113287046-113286876:1;1706/chr4:1664:chr4:40859069-40858900:2;1707/chr4:1664:chr
4:40859069-40858900:1;1708/chr10:4357:chr10:133796207-133796055:2;1709/chr10:4357:chr10:133796207-1
33796055:1;1710/chr13:1700:chr13:113764226-113764055:2;1711/chr13:1700:chr13:113764226-113764055:1;
1712/chr1:7210:chr1:237206577-237206452:2;1713/chr1:7210:chr1:237206577-237206452:1;1714/chr14:454:c
hr14:48144169-48144013:2;1715/chr14:454:chr14:48144169-48144013:1;1716/chr8:1720:chr8:54791995-5479
1849:2;1717/chr8:1720:chr8:54791995-54791849:1;1718/chr10:4591:chr10:134602170-134602045:2;1719/chr1
2:2984:chr12:129338495-129338347:2;1720/chr12:2984:chr12:129338495-129338347:1;1721/chr15:264:chr15:
28352965-28352820:1;1722/chr19:4708:chr19:55997858-55997686:2;1723/chr19:4708:chr19:55997858-55997
686:1;1724/chr10:4591:chr10:134602170-134602045:1;1725/chr1:59:chr1:969637-969505:2;1726/chr1:59:chr1:
969637-969505:1;1727/chr3:1095:chr3:47050951-47050803:1;1728/chr17:4139:chr17:77789075-77788900:2;1
729/chr17:4139:chr17:77789075-77788900:1;1730/chr10:4583:chr10:134599436-134599278:2;1731/chr10:458
3:chr10:134599436-134599278:1;1732/chr1:3916:chr1:46951560-46951399:1;1733/chr15:1558:chr15:7663951
8-76639393:1;1734/chr4:207:chr4:1400795-1400625:1;1735/chr15:1832:chr15:83953429-83953303:2;1736/chr
15:1832:chr15:83953429-83953303:1;1737/chr15:2011:chr15:89950895-89950720:1;1738/chr7:2696:chr7:7284
8359-72848210:2;1739/chr7:2696:chr7:72848359-72848210:1;1740/chr19:2540:chr19:30017532-30017403:2;1
741/chr19:2540:chr19:30017532-30017403:1;1742/chr22:1852:chr22:46475957-46475784:1;1743/chr20:2210:c
hr20:59827778-59827622:2;1744/chr20:2210:chr20:59827778-59827622:1;1745/chr11:4678:chr11:130029753-
130029582:2;1746/chr11:4678:chr11:130029753-130029582:1;1747/chr19:4708:chr19:55997910-55997742:1;1
748/chr11:141:chr11:849717-849574:2;1749/chr11:141:chr11:849717-849574:1;1750/chr9:1770:chr9:12726597
1-127265845:2;1751/chr9:1770:chr9:127265971-127265845:1;1752/chr17:1003:chr17:11143920-11143748:2;1
753/chr17:1003:chr17:11143920-11143748:1;1754/chr7:2651:chr7:71801243-71801070:2;1755/chr7:2651:chr7:
71801243-71801070:1;1756/chr13:1461:chr13:112723112-112722941:2;1757/chr13:1461:chr13:112723112-11
2722941:1;1758/cg17872757:chr11:128564260-128564128:1;1759/chr20:525:chr20:21377495-21377361:2;176
0/chr20:525:chr20:21377495-21377361:1;1761/chr21:158:chr21:32931842-32931715:2;1762/chr21:158:chr21:3
2931842-32931715:1;1763/chr17:1558:chr17:28088686-28088561:2;1764/chr17:1558:chr17:28088686-280885
61:1;1765/chr17:2375:chr17:46711323-46711149:1;1766/chr20:1728:chr20:50721804-50721640:2;1767/chr20:
1728:chr20:50721804-50721640:1;1768/chr10:848:chr10:21799485-21799313:2;1769/chr10:848:chr10:217994
85-21799313:1;1770/chr8:1309:chr8:35092911-35092759:2;1771/chr8:1309:chr8:35092911-35092759:1;1772/c
hr14:1433:chr14:93153804-93153675:2;1773/chr14:1433:chr14:93153804-93153675:1;1774/chr1:3209:chr1:29
448016-29447851:2;1775/chr1:3209:chr1:29448016-29447851:1;1776/chr10:2308:chr10:88123173-88123004:2;
1777/chr10:2308:chr10:88123173-88123004:1;1778/chr12:428:chr12:6665547-6665382:2;1779/chr12:428:chr1
2:6665547-6665382:1;1780/chr19:759:chr19:3435327-3435160:2;1781/chr19:759:chr19:3435327-3435160:1;17
82/chr9:1226:chr9:100617021-100616896:2;1783/chr9:1226:chr9:100617021-100616896:1;1784/chr5:1812:chr
5:77147725-77147574:2;1785/chr5:1812:chr5:77147725-77147574:1;1786/chr11:461:chr11:1864283-1864143:
1;1787/chr7:3616:chr7:130419074-130418920:2;1788/chr1:5370:chr1:156046785-156046621:2;1789/chr1:5370:
chr1:156046785-156046621:1;1790/chr7:3586:chr7:129421859-129421689:2;1791/chr7:3586:chr7:129421859-
129421689:1;1792/chr1:3956:chr1:47904618-47904456:2;1793/chr1:3956:chr1:47904618-47904456:1;1794/chr
19:325:chr19:1302648-1302474:2;1795/chr19:325:chr19:1302648-1302474:1;1796/chr9:239:chr9:23822085-23
821911:2;1797/chr9:239:chr9:23822085-23821911:1;1798/chr17:4170:chr17:77916835-77916670:2;1799/chr4:
1487:chr4:21950414-21950243:2;1800/chr4:1487:chr4:21950414-21950243:1;1801/chr20:2056:chr20:5776652
7-57766364:1;1802/chr15:2063:chr15:90543355-90543230:2;1803/chr16:3215:chr16:77468803-77468637:2;18
04/chr16:3215:chr16:77468803-77468637:1;1805/chr7:811:chr7:2968628-2968453:2;1806/chr7:811:chr7:29686
28-2968453:1;1807/chr19:566:chr19:2290324-2290176:2;1808/chr19:566:chr19:2290324-2290176:1;1809/chr1
9:1807:chr19:14583177-14583049:2;1810/chr19:1807:chr19:14583177-14583049:1;1811/chr1:2257:chr1:13840
120-13839952:2;1812/chr1:2257:chr1:13840120-13839952:1;1813/chr7:1486:chr7:27141825-27141678:2;1814/
chr7:1486:chr7:27141825-27141678:1;1815/chr10:4051:chr10:131757340-131757165:2;1816/chr10:4051:chr10:
131757340-131757165:1;1817/chr17:4170:chr17:77916835-77916670:1;1818/chr6:2558:chr6:137810073-1378
09914:2;1819/chr6:2558:chr6:137810073-137809914:1;1820/chr15:2063:chr15:90543355-90543230:1;1821/chr
9:3136:chr9:138606326-138606158:2;1822/chr9:3136:chr9:138606326-138606158:1;1823/chr2:1181:chr2:2423
3090-24232939:2;1824/chr2:1181:chr2:24233090-24232939:1;1825/chr6:2142:chr6:100061973-100061801:2;1
826/chr6:2142:chr6:100061973-100061801:1;1827/chr4:2084:chr4:85419112-85418937:2;1828/chr4:2084:chr4:
85419112-85418937:1;1829/chr6:1567:chr6:42072744-42072604:1;1830/chr17:1003:chr17:11144720-1114454
5:2;1831/chr17:1003:chr17:11144720-11144545:1;1832/chr12:1613:chr12:72666345-72666215:2;1833/chr12:1
613:chr12:72666345-72666215:1;1834/chr8:1962:chr8:70981968-70981825:1;1835/chr19:1690:chr19:1320755
9-13207385:2;1836/chr5:2942:chr5:159399522-159399381:2;1837/chr1:5180:chr1:151810939-151810798:2;18
38/chr2:3718:chr2:131797753-131797611:2;1839/chr5:1719:chr5:72678461-72678298:2;1840/chr5:1719:chr5:7
2678461-72678298:1;1841/chr22:2764:chr22:50987389-50987218:2;1842/chr22:2764:chr22:50987389-509872
18:1;1843/chr2:4182:chr2:175199882-175199708:2;1844/chr2:4182:chr2:175199882-175199708:1;1845/chr7:3
489:chr7:126988120-126987957:2;1846/chr7:3489:chr7:126988120-126987957:1;1847/chr7:2162:chr7:503439
16-50343764:2;1848/chr8:1742:chr8:55379682-55379513:2;1849/chr8:1742:chr8:55379682-55379513:1;1850/c
hr20:1816:chr20:55205853-55205711:2;1851/chr20:1816:chr20:55205853-55205711:1;1852/chr19:3503:chr19:
41119748-41119577:1;1853/chr18:1155:chr18:70534824-70534686:2;1854/chr18:1155:chr18:70534824-70534
686:1;1855/chr16:1606:chr16:23847789-23847616:2;1856/chr16:1606:chr16:23847789-23847616:1;1857/chr7:
2162:chr7:50343916-50343764:1;1858/chr2:5862:chr2:242498352-242498227:2;1859/chr2:5862:chr2:2424983
52-242498227:1;1860/chr11:2512:chr11:65553881-65553714:2;1861/chr11:2512:chr11:65553881-65553714:1;
1862/chr14:125:chr14:24045711-24045561:2;1863/chr14:125:chr14:24045711-24045561:1;1864/chr2:4252:chr
2:176987999-176987831:2;1865/chr2:4252:chr2:176987999-176987831:1;1866/chr19:1690:chr19:13207559-13
207385:1;1867/chr4:5:chr4:107479-107323:2;1868/chr4:5:chr4:107479-107323:1;1869/chr19:368:chr19:146951
2-1469347:2;1870/chr19:368:chr19:1469512-1469347:1;1871/chr5:2942:chr5:159399522-159399381:1;1872/ch
r1:5180:chr1:151810939-151810798:1;1873/chr2:3718:chr2:131797753-131797611:1;1874/chr22:720:chr22:30
662103-30661932:2;1875/chr22:720:chr22:30662103-30661932:1;1876/chr7:1520:chr7:27196057-27195893:2;
1877/chr7:1520:chr7:27196057-27195893:1;1878/chr20:2983:chr20:62369510-62369349:2;1879/chr20:2983:ch
r20:62369510-62369349:1;1880/chr4:2712:chr4:174428165-174427996:2;1881/chr4:2712:chr4:174428165-174
427996:1;1882/chrX:666:chrX:39864731-39864593:2;1883/chrX:666:chrX:39864731-39864593:1;1884/chr14:2
078:chr14:102027737-102027611:2;1885/chr14:2078:chr14:102027737-102027611:1;1886/chr19:3997:chr19:4
8946521-48946346:2;1887/chr19:3997:chr19:48946521-48946346:1;1888/chr10:3445:chr10:124896849-12489
6687:2;1889/chr10:3445:chr10:124896849-124896687:1;1890/chr4:62:chr4:682961-682811:2;1891/chr4:62:chr
4:682961-682811:1;1892/chr15:2554:chr15:102029749-102029590:2;1893/chr15:2554:chr15:102029749-10202
9590:1;1894/chr17:2799:chr17:59474665-59474503:2;1895/chr17:2799:chr17:59474665-59474503:1;1896/chr1
6:660:chr16:2229076-2228944:2;1897/chr16:660:chr16:2229076-2228944:1;1898/chr7:4565:chr7:155744500-1
55744326:2;1899/chr7:4565:chr7:155744500-155744326:1;1900/chr13:452:chr13:31480856-31480703:2;1901/
chr13:452:chr13:31480856-31480703:1;1902/chr11:4451:chr11:124790958-124790807:1;1903/chr15:1535:chr1
5:76629188-76629025:2;1904/chr15:1535:chr15:76629188-76629025:1;1905/chr17:3473:chr17:73584294-7358
4119:2;1906/chr17:3473:chr17:73584294-73584119:1;1907/chr4:470:chr4:2820162-2820005:2;1908/chr4:470:c
hr4:2820162-2820005:1;1909/chr19:4895:chr19:58545695-58545521:2;1910/chr19:4895:chr19:58545695-5854
5521:1;1911/chr14:1947:chr14:101193462-101193337:2;1912/chr14:1947:chr14:101193462-101193337:1;1913/
chr1:7412:chr1:248020749-248020619:2;1914/chr1:7412:chr1:248020749-248020619:1;1915/chr13:1461:chr1
3:112723393-112723261:2;1916/chr13:1461:chr13:112723393-112723261:1;1917/chr1:831:chr1:2986962-2986
797:2;1918/chr1:831:chr1:2986962-2986797:1;1919/chr2:4739:chr2:219866229-219866058:2;1920/chr2:4739:c
hr2:219866229-219866058:1;1921/chr16:1968:chr16:31488955-31488787:2;1922/chr16:1968:chr16:31488955-
31488787:1;1923/chr9:1287:chr9:102590929-102590756:2;1924/chr9:1287:chr9:102590929-102590756:1;1925/
chr10:519:chr10:8094233-8094108:2;1926/chr10:519:chr10:8094233-8094108:1;1927/chr2:1990:chr2:6667252
6-66672387:2;1928/chr2:1990:chr2:66672526-66672387:1;1929/chr12:1258:chr12:54071195-54071036:2;1930/
chr12:1258:chr12:54071195-54071036:1;1931/chr2:3108:chr2:115919923-115919794:2;1932/chr2:3108:chr2:1
15919923-115919794:1;1933/chr18:97:chr18:5629938-5629764:2;1934/chr18:97:chr18:5629938-5629764:1;19
35/chr6:852:chr6:28945620-28945480:2;1936/chr6:852:chr6:28945620-28945480:1;1937/chr18:1256:chr18:736
28494-73628339:2;1938/chr18:1256:chr18:73628494-73628339:1;1939/chr20:2719:chr20:61703557-61703391:
2;1940/chr20:2719:chr20:61703557-61703391:1;1941/chr17:3502:chr17:73750240-73750069:2;1942/chr17:350
2:chr17:73750240-73750069:1;1943/chr11:716:chr11:2291567-2291397:2;1944/chr11:716:chr11:2291567-2291
397:1;1945/cg20154403:chr19:37407492-37407337:1;1946/chr13:1082:chr13:100649818-100649655:2;1947/c
hr13:1082:chr13:100649818-100649655:1;1948/chr4:1280:chr4:8874806-8874635:2;1949/chr4:1280:chr4:8874
806-8874635:1;1950/chr19:362:chr19:1457468-1457342:2;1951/chr13:521:chr13:37248320-37248159:2;1952/c
hr13:521:chr13:37248320-37248159:1;1953/chr8:2423:chr8:120220948-120220818:2;1954/chr8:2423:chr8:120
220948-120220818:1;1955/chr19:362:chr19:1457468-1457342:1;1956/chr18:1046:chr18:56941627-56941462:2;
1957/chr18:1046:chr18:56941627-56941462:1;1958/chr8:37:chr8:686920-686787:2;1959/chr8:37:chr8:686920-
686787:1;1960/chr8:738:chr8:11559838-11559700:2;1961/chr15:983:chr15:60297221-60297078:2;1962/chr15:
983:chr15:60297221-60297078:1;1963/chr1:3196:chr1:29101737-29101588:1;1964/chr8:656:chr8:10590098-10
589937:2;1965/chr8:656:chr8:10590098-10589937:1;1966/chr2:741:chr2:8817104-8816971:2;1967/chr2:741:ch
r2:8817104-8816971:1;1968/chr22:1308:chr22:41048689-41048519:2;1969/chr22:1308:chr22:41048689-41048
519:1;1970/chr2:5245:chr2:237082219-237082094:2;1971/chr2:5245:chr2:237082219-237082094:1;1972/chr8:
738:chr8:11559838-11559700:1;1973/chr6:1651:chr6:43970554-43970394:2;1974/chr8:3703:chr8:145105961-1
45105794:2;1975/chr8:3703:chr8:145105961-145105794:1;1976/chr6:1651:chr6:43970554-43970394:1;1977/c
hr10:2676:chr10:102507820-102507695:2;1978/chr10:2676:chr10:102507820-102507695:1;1979/chr12:51:chr1
2:679949-679781:2;1980/chr12:51:chr12:679949-679781:1;1981/chr20:1038:chr20:36012930-36012762:2;1982/
chr20:1038:chr20:36012930-36012762:1;1983/chr9:193:chr9:17579449-17579282:2;1984/chr9:193:chr9:17579
449-17579282:1;1985/chr17:2797:chr17:59473663-59473493:2;1986/chr17:2797:chr17:59473663-59473493:1;
1987/chr9:207:chr9:19127643-19127508:2;1988/chr9:207:chr9:19127643-19127508:1;1989/chr14:212:chr14:27
067457-27067296:2;1990/chr14:212:chr14:27067457-27067296:1;1991/chr5:1055:chr5:7850130-7849961:2;19
92/chr5:1055:chr5:7850130-7849961:1;1993/chr5:2559:chr5:140871584-140871410:2;1994/chr5:2559:chr5:140
871584-140871410:1;1995/chr10:3406:chr10:123922968-123922801:2;1996/chr10:3406:chr10:123922968-123
922801:1;1997/chr1:7175:chr1:235814087-235813930:2;1998/chr1:7175:chr1:235814087-235813930:1;1999/c
hr17:1218:chr17:17399245-17399076:2;2000/chr17:1218:chr17:17399245-17399076:1;2001/chr20:524:chr20:2
1376499-21376324:2;2002/chr20:524:chr20:21376499-21376324:1;2003/chr16:1850:chr16:29888423-2988825
1:2;2004/chr16:1850:chr16:29888423-29888251:1;2005/chr21:666:chr21:44037522-44037372:1;2006/chr14:34
1:chr14:37053387-37053220:2;2007/chr14:341:chr14:37053387-37053220:1;2008/chr7:3501:chr7:127672295-1
27672163:2;2009/chr7:3501:chr7:127672295-127672163:1;2010/chr13:1702:chr13:113764827-113764660:2;20
11/chr15:1417:chr15:74425719-74425593:2;2012/chr15:1417:chr15:74425719-74425593:1;2013/chr2:1902:chr
2:63274880-63274709:2;2014/chr2:1902:chr2:63274880-63274709:1;2015/chr19:3999:chr19:48947276-489471
49:2;2016/chr19:3999:chr19:48947276-48947149:1;2017/chr15:201:chr15:27017671-27017545:2;2018/chr15:2
01:chr15:27017671-27017545:1;2019/chr2:2063:chr2:71116196-71116030:2;2020/chr2:2063:chr2:71116196-71
116030:1;2021/chr13:1702:chr13:113764827-113764660:1;2022/chr13:1468:chr13:112727861-112727691:2;20
23/chr13:1468:chr13:112727861-112727691:1;2024/chr12:1318:chr12:54427146-54427021:2;2025/chr12:1318:
chr12:54427146-54427021:1;2026/chr5:2324:chr5:134364064-134363929:2;2027/chr5:2324:chr5:134364064-1
34363929:1;2028/chr5:2254:chr5:132161540-132161380:2;2029/chr5:2254:chr5:132161540-132161380:1;2030/
chr1:3435:chr1:34642568-34642402:2;2031/chr1:3435:chr1:34642568-34642402:1;2032/chr5:1614:chr5:60922
327-60922152:2;2033/chr5:1614:chr5:60922327-60922152:1;2034/chr5:1926:chr5:92906541-92906387:2;2035/
chr5:1926:chr5:92906541-92906387:1;2036/chr3:3063:chr3:179754937-179754800:2;2037/chr3:3063:chr3:179
754937-179754800:1;2038/chr11:4451:chr11:124791078-124790904:2;2039/chr11:4451:chr11:124791078-124
790904:1;2040/chr19:4796:chr19:56988748-56988623:2;2041/chr19:4796:chr19:56988748-56988623:1;2042/c
hr11:2133:chr11:61277193-61277028:2;2043/chr11:2133:chr11:61277193-61277028:1;2044/chr9:2143:chr9:13
2651033-132650862:2;2045/chr9:2143:chr9:132651033-132650862:1;2046/chr9:1080:chr9:96721017-9672085
0:2;2047/chr9:1080:chr9:96721017-96720850:1;2048/chr1:252:chr1:1565757-1565583:2;2049/chr1:252:chr1:15
65757-1565583:1;2050/chr1:5180:chr1:151811126-151810980:2;2051/chr1:5180:chr1:151811126-151810980:1;
2052/chr17:1316:chr17:19628259-19628097:2;2053/chr17:1316:chr17:19628259-19628097:1;2054/chr6:105:c
hr6:1393360-1393202:2;2055/chr6:105:chr6:1393360-1393202:1;2056/chrX:784:chrX:47433679-47433504:2;20
57/chrX:784:chrX:47433679-47433504:1;2058/cg13648501:chr5:142785401-142785227:1;2059/chr22:1894:chr
22:46658821-46658647:2;2060/chr22:1894:chr22:46658821-46658647:1;2061/chr16:2389:chr16:51186650-511
86483:2;2062/chr16:2389:chr16:51186650-51186483:1;2063/chr16:3092:chr16:73097048-73096882:2;2064/chr
16:3092:chr16:73097048-73096882:1;2065/chr21:861:chr21:45577597-45577453:1;2066/chr13:356:chr13:2854
9930-28549805:2;2067/chr13:356:chr13:28549930-28549805:1;2068/chr3:762:chr3:32859418-32859245:2;206
9/chr9:1374:chr9:112081646-112081476:2;2070/chr9:1374:chr9:112081646-112081476:1;2071/chr3:1771:chr3:
73673886-73673714:2;2072/chr20:3023:chr20:62463531-62463392:2;2073/chr20:3023:chr20:62463531-62463
392:1;2074/chr8:2241:chr8:99961013-99960887:2;2075/chr8:2241:chr8:99961013-99960887:1;2076/chr3:1771:
chr3:73673886-73673714:1;2077/chr19:1699:chr19:13215597-13215426:2;2078/chr19:1699:chr19:13215597-1
3215426:1;2079/cg05894124:chr4:81186830-81186655:1;2080/chr19:1946:chr19:16436418-16436244:2;2081/
chr22:1367:chr22:42679589-42679463:2;2082/chr22:1367:chr22:42679589-42679463:1;2083/chr5:514:chr5:18
87550-1887384:2;2084/chr5:514:chr5:1887550-1887384:1;2085/chr19:566:chr19:2290446-2290321:2;2086/chr
19:566:chr19:2290446-2290321:1;2087/chr6:458:chr6:12750408-12750283:1;2088/chr9:1083:chr9:96722731-9
6722591:2;2089/chr9:1083:chr9:96722731-96722591:1;2090/cg19897940:chr11:128563741-128563566:1;2091/
cg13755070:chr11:128563741-128563566:1;2092/chr8:2179:chr8:97157246-97157113:2;2093/chr8:2179:chr8:
97157246-97157113:1;2094/chr8:42:chr8:688944-688784:2;2095/chr8:42:chr8:688944-688784:1;2096/chr5:245
0:chr5:138730101-138729956:2;2097/chr5:2450:chr5:138730101-138729956:1;2098/chr13:289:chr13:2662585
6-26625713:2;2099/chr17:1316:chr17:19627962-19627832:2;2100/chr17:1316:chr17:19627962-19627832:1;21
01/chr4:2434:chr4:140656830-140656659:2;2102/chr4:2434:chr4:140656830-140656659:1;2103/chr13:289:chr
13:26625856-26625713:1;2104/chr14:1682:chr14:97499784-97499619:2;2105/chr14:1682:chr14:97499784-974
99619:1;2106/chr2:3996:chr2:162279999-162279833:2;2107/chr2:3996:chr2:162279999-162279833:1;2108/chr
1:3496:chr1:36772265-36772094:2;2109/chr1:3496:chr1:36772265-36772094:1;2110/chr3:5:chr3:238818-2386
55:2;2111/chr3:5:chr3:238818-238655:1;2112/chr2:118:chr2:946626-946498:2;2113/chr2:118:chr2:946626-946
498:1;2114/chr8:1788:chr8:57358424-57358277:2;2115/chr8:1788:chr8:57358424-57358277:1;2116/chr19:407
5:chr19:49576171-49576037:2;2117/chr19:4075:chr19:49576171-49576037:1;2118/chr8:3703:chr8:145106221-
145106096:2;2119/chr8:3703:chr8:145106221-145106096:1;2120/chr19:4862:chr19:58038992-58038821:2;212
1/chr19:4862:chr19:58038992-58038821:1;2122/chr3:762:chr3:32859418-32859245:1;2123/chr2:3031:chr2:111
876887-111876722:2;2124/chr2:3031:chr2:111876887-111876722:1;2125/chr19:1946:chr19:16436418-164362
44:1;2126/chr9:1226:chr9:100617372-100617224:2;2127/chr9:1226:chr9:100617372-100617224:1;2128/chr11:
1094:chr11:7695189-7695022:2;2129/chr11:1094:chr11:7695189-7695022:1;2130/chr18:1140:chr18:67068544-
67068411:2;2131/chr18:1140:chr18:67068544-67068411:1;2132/chr3:3517:chr3:196256098-196255933:1;2133/
chr10:846:chr10:21789396-21789223:2;2134/chr10:846:chr10:21789396-21789223:1;2135/chr4:207:chr4:1400
965-1400833:2;2136/chr4:207:chr4:1400965-1400833:1;2137/chr12:2984:chr12:129338396-129338238:2;2138/
chr12:2984:chr12:129338396-129338238:1;2139/chr7:3112:chr7:100091504-100091379:2;2140/chr7:3112:chr7:
100091504-100091379:1;2141/chr10:1133:chr10:35929187-35929052:2;2142/chr10:1133:chr10:35929187-359
29052:1;2143/chr16:3092:chr16:73096989-73096817:2;2144/chr16:3092:chr16:73096989-73096817:1;2145/chr
4:1481:chr4:20256936-20256773:2;2146/chr4:1481:chr4:20256936-20256773:1;2147/chr1:6929:chr1:22860417
0-228604000:2;2148/chr1:6929:chr1:228604170-228604000:1;2149/chr8:2241:chr8:99960735-99960610:2;215
0/chr8:2241:chr8:99960735-99960610:1;2151/chr8:1018:chr8:22446986-22446819:2;2152/chr8:1018:chr8:2244
6986-22446819:1;2153/chr6:1788:chr6:54711556-54711416:2;2154/chr6:1788:chr6:54711556-54711416:1;215
5/chr22:2705:chr22:50629239-50629099:2;2156/chr22:2705:chr22:50629239-50629099:1;2157/chr10:1049:chr
10:30024128-30024001:2;2158/chr10:1049:chr10:30024128-30024001:1;2159/chr4:1509:chr4:24801714-24801
588:2;2160/chr4:1509:chr4:24801714-24801588:1;2161/chr19:2725:chr19:31839783-31839634:2;2162/chr19:2
725:chr19:31839783-31839634:1;2163/chr14:761:chr14:65210288-65210120:1;2164/chr9:3295:chr9:13909375
4-139093592:2;2165/chr9:3295:chr9:139093754-139093592:1;2166/chr17:965:chr17:10101721-10101546:2;21
67/chr17:965:chr17:10101721-10101546:1;2168/chr5:829:chr5:3595594-3595419:2;2169/chr5:829:chr5:359559
4-3595419:1;2170/chr20:1991:chr20:57224798-57224633:2;2171/chr20:1991:chr20:57224798-57224633:1;217
2/chr5:1190:chr5:16180094-16179920:2;2173/chr5:1190:chr5:16180094-16179920:1;2174/chr1:7175:chr1:2358
14031-235813859:2;2175/chr1:7175:chr1:235814031-235813859:1;2176/chr13:982:chr13:96294647-96294495:
2;2177/chr13:982:chr13:96294647-96294495:1;2178/chr8:650:chr8:10587741-10587582:2;2179/chr8:650:chr8:
10587741-10587582:1;2180/chr5:535:chr5:1931448-1931286:2;2181/chr5:535:chr5:1931448-1931286:1;2182/c
hr11:4451:chr11:124791311-124791151:2;2183/chr11:4451:chr11:124791311-124791151:1;2184/chr1:3371:chr
1:32828107-32827982:2;2185/chr1:3371:chr1:32828107-32827982:1;2186/chr18:1360:chr18:74962552-749623
98:2;2187/chr18:1360:chr18:74962552-74962398:1;2188/chr5:3534:chr5:177540862-177540706:2;2189/chr5:3
534:chr5:177540862-177540706:1;2190/chr13:356:chr13:28550147-28549986:2;2191/chr13:356:chr13:285501
47-28549986:1;2192/chr2:2063:chr2:71116119-71115961:2;2193/chr2:2063:chr2:71116119-71115961:1;2194/c
hr2:5245:chr2:237081989-237081834:2;2195/chr2:5245:chr2:237081989-237081834:1;2196/chr4:56:chr4:6759
70-675805:2;2197/chr4:56:chr4:675970-675805:1;2198/chr19:1577:chr19:11689972-11689797:2;2199/chr14:26
62:chr14:105553375-105553205:2;2200/chr14:2662:chr14:105553375-105553205:1;2201/chr19:3873:chr19:46
997042-46996898:2;2202/chr19:3873:chr19:46997042-46996898:1;2203/chr15:935:chr15:58357280-58357155:
2;2204/chr15:935:chr15:58357280-58357155:1;2205/chr19:3157:chr19:37096069-37095941:2;2206/chr19:3157:
chr19:37096069-37095941:1;2207/chr13:236:chr13:25320207-25320065:2;2208/chr13:236:chr13:25320207-25
320065:1;2209/chr1:4824:chr1:110611655-110611485:2;2210/chr1:4824:chr1:110611655-110611485:1;2211/c
hr14:761:chr14:65210188-65210035:1;2212/chr5:1718:chr5:72677831-72677688:1;2213/chr5:3916:chr5:18001
8422-180018295:2;2214/chr5:3916:chr5:180018422-180018295:1;2215/chr14:157:chr14:24780464-24780321:2;
2216/chr14:157:chr14:24780464-24780321:1;2217/chr20:476:chr20:20346256-20346131:2;2218/chr20:476:chr
20:20346256-20346131:1;2219/chr18:1611:chr18:76739890-76739715:2;2220/chr18:1611:chr18:76739890-767
39715:1;2221/chr9:55:chr9:1045795-1045621:2;2222/chr15:2253:chr15:95388723-95388583:1;2223/chr9:1218:
chr9:100611133-100610988:1;2224/chr20:2928:chr20:62119869-62119697:2;2225/chr20:2928:chr20:62119869-
62119697:1;2226/cg14732998:chr19:37997948-37997775:1;2227/cg04977834:chr17:70114728-70114568:1;2
228/chr22:1921:chr22:46932455-46932290:2;2229/chr18:966:chr18:53447721-53447546:2;2230/chr18:966:chr
18:53447721-53447546:1;2231/chr14:322:chr14:36986961-36986799:2;2232/chr14:322:chr14:36986961-36986
799:1;2233/chr13:657:chr13:49795354-49795220:2;2234/chr13:657:chr13:49795354-49795220:1;2235/chr4:29
81:chr4:189203933-189203759:1;2236/chr14:387:chr14:38064459-38064284:2;2237/chr14:387:chr14:3806445
9-38064284:1;2238/chr7:811:chr7:2968715-2968540:2;2239/chr7:811:chr7:2968715-2968540:1;2240/chr8:42:c
hr8:689209-689063:2;2241/chr20:1878:chr20:55965620-55965464:2;2242/chr8:42:chr8:689209-689063:1;2243/
chr14:229:chr14:29236491-29236319:1;2244/chr17:965:chr17:10101749-10101612:2;2245/chr17:965:chr17:10
101749-10101612:1;2246/chr2:4131:chr2:172964990-172964837:2;2247/chr2:4131:chr2:172964990-17296483
7:1;2248/chr16:2476:chr16:54971240-54971069:2;2249/chr16:2476:chr16:54971240-54971069:1;2250/chr22:1
248:chr22:40058246-40058099:2;2251/chr22:1248:chr22:40058246-40058099:1;2252/chr6:92:chr6:1314071-13
13903:2;2253/chr6:92:chr6:1314071-1313903:1;2254/chr19:3321:chr19:39087049-39086878:2;2255/cg140634
88:chr4:154709962-154709789:2;2256/chr8:40:chr8:688238-688094:2;2257/chr8:40:chr8:688238-688094:1;225
8/chr19:4173:chr19:50393822-50393661:2;2259/chr19:4173:chr19:50393822-50393661:1;2260/chr12:3097:chr
12:130648364-130648191:2;2261/chr16:693:chr16:2392209-2392043:2;2262/chr16:693:chr16:2392209-239204
3:1;2263/chr19:1696:chr19:13210443-13210306:2;2264/chr19:1696:chr19:13210443-13210306:1;2265/chr7:25
89:chr7:70597010-70596879:2;2266/chr7:2589:chr7:70597010-70596879:1;2267/chr19:369:chr19:1470457-147
0288:2;2268/chr8:3776:chr8:145806482-145806341:2;2269/chr8:3776:chr8:145806482-145806341:1;2270/chr6:
138:chr6:1614739-1614564:2;2271/chr7:3421:chr7:115850724-115850555:2;2272/chr7:3421:chr7:115850724-
115850555:1;2273/chr17:3789:chr17:76127531-76127386:2;2274/chr16:393:chr16:1211002-1210828:2;2275/c
hr17:3789:chr17:76127531-76127386:1;2276/chr15:2003:chr15:89922511-89922386:2;2277/chr15:2003:chr15:
89922511-89922386:1;2278/chr5:1488:chr5:50695381-50695212:1;2279/chr19:2895:chr19:34113185-3411302
1:2;2280/chr19:2895:chr19:34113185-34113021:1;2281/chr17:2535:chr17:48546291-48546116:2;2282/chr17:2
535:chr17:48546291-48546116:1;2283/chr15:275:chr15:29131172-29131010:2;2284/chr15:275:chr15:2913117
2-29131010:1;2285/chr14:1633:chr14:96343605-96343434:2;2286/chr14:1633:chr14:96343605-96343434:1;22
87/chr1:831:chr1:2986898-2986760:2;2288/chr8:3309:chr8:143545744-143545576:2;2289/chr1:831:chr1:29868
98-2986760:1;2290/chr13:85:chr13:20875956-20875812:2;2291/chr13:85:chr13:20875956-20875812:1;2292/ch
r19:3321:chr19:39087049-39086878:1;2293/cg14063488:chr4:154709962-154709789:1;2294/chr15:428:chr15:
31775864-31775732:2;2295/chr15:428:chr15:31775864-31775732:1;2296/chr12:3097:chr12:130648364-13064
8191:1;2297/chr14:2078:chr14:102027359-102027205:2;2298/chr14:2078:chr14:102027359-102027205:1;2299/
chr19:369:chr19:1470457-1470288:1;2300/chr14:627:chr14:59932292-59932118:2;2301/chr14:627:chr14:5993
2292-59932118:1;2302/chr6:138:chr6:1614739-1614564:1;2303/chr13:870:chr13:79176685-79176553:2;2304/c
hr13:870:chr13:79176685-79176553:1;2305/chr12:1457:chr12:58120498-58120333:2;2306/chr12:1457:chr12:5
8120498-58120333:1;2307/chr11:4166:chr11:119211747-119211591:1;2308/chr19:4874:chr19:58238801-5823
8653:2;2309/chr19:4874:chr19:58238801-58238653:1;2310/chr20:946:chr20:32857033-32856874:2;2311/chr20:
946:chr20:32857033-32856874:1;2312/chr3:1158:chr3:48699451-48699308:2;2313/chr3:1158:chr3:48699451-
48699308:1;2314/chr21:324:chr21:38077002-38076829:2;2315/chr21:324:chr21:38077002-38076829:1;2316/c
hr15:937:chr15:58357884-58357758:2;2317/chr15:937:chr15:58357884-58357758:1;2318/chr15:1417:chr15:74
426014-74425875:2;2319/chr15:1417:chr15:74426014-74425875:1;2320/chr10:3180:chr10:118899951-118899
778:1;2321/chr1:3953:chr1:47899807-47899633:2;2322/chr1:3953:chr1:47899807-47899633:1;2323/chr4:1403:
chr4:13544317-13544188:1;2324/chr4:1277:chr4:8873263-8873134:2;2325/chr4:1277:chr4:8873263-8873134:1;
2326/chr1:3150:chr1:27687495-27687333:2;2327/chr1:3150:chr1:27687495-27687333:1;2328/chr4:5:chr4:107
968-107807:2;2329/chr4:5:chr4:107968-107807:1;2330/chr9:629:chr9:79635631-79635477:2;2331/chr2:4189:c
hr2:175202290-175202156:2;2332/chr2:4189:chr2:175202290-175202156:1;2333/chr10:4068:chr10:131771311-
131771167:2;2334/chr10:4068:chr10:131771311-131771167:1;2335/chr18:487:chr18:22929844-22929696:2;2
336/chr10:2949:chr10:108924567-108924400:2;2337/chr17:537:chr17:4804329-4804197:2;2338/chr17:537:chr
17:4804329-4804197:1;2339/chr9:1188:chr9:99482461-99482320:2;2340/chr9:1188:chr9:99482461-99482320:
1;2341/chr16:2827:chr16:66613416-66613250:2;2342/chr16:2827:chr16:66613416-66613250:1;2343/chr5:2364:
chr5:134871181-134871007:2;2344/chr5:2364:chr5:134871181-134871007:1;2345/chr15:2011:chr15:8995115
5-89951009:2;2346/chr15:2011:chr15:89951155-89951009:1;2347/chr11:3664:chr11:94277790-94277617:2;23
48/chr1:3845:chr1:44704224-44704069:2;2349/chr1:3845:chr1:44704224-44704069:1;2350/chr2:1925:chr2:632
86370-63286198:2;2351/chr3:2518:chr3:133393625-133393497:2;2352/chr3:2518:chr3:133393625-133393497:
1;2353/chr2:4945:chr2:231693141-231693011:2;2354/chr2:4945:chr2:231693141-231693011:1;2355/chr2:1925:
chr2:63286370-63286198:1;2356/chr8:40:chr8:688484-688312:2;2357/chr8:40:chr8:688484-688312:1;2358/chr
11:3664:chr11:94277790-94277617:1;2359/chr9:1980:chr9:130689715-130689590:2;2360/chr9:1980:chr9:1306
89715-130689590:1;2361/chr8:3701:chr8:145104144-145104019:2;2362/chr8:3701:chr8:145104144-14510401
9:1;2363/chr8:2051:chr8:80804177-80804024:2;2364/chr7:3315:chr7:103630125-103630000:2;2365/chr7:3315:
chr7:103630125-103630000:1;2366/chr9:629:chr9:79635631-79635477:1;2367/chr16:726:chr16:2835441-2835
301:2;2368/chr16:726:chr16:2835441-2835301:1;2369/chr13:1184:chr13:109793087-109792920:2;2370/chr13:
1184:chr13:109793087-109792920:1;2371/chr19:2231:chr19:18980752-18980624:2;2372/chr19:2231:chr19:18
980752-18980624:1;2373/chr7:3112:chr7:100091571-100091421:2;2374/chr7:3112:chr7:100091571-10009142
1:1;2375/chr5:2195:chr5:127873559-127873397:2;2376/chr5:2195:chr5:127873559-127873397:1;2377/chr14:2
086:chr14:102057195-102057023:1;2378/chr19:3998:chr19:48946725-48946585:2;2379/chr19:3998:chr19:489
46725-48946585:1;2380/cg03298319:chr14:33403125-33402950:1;2381/chr16:2445:chr16:54324557-5432443
0:2;2382/chr2:5386:chr2:239072810-239072648:2;2383/chr2:5386:chr2:239072810-239072648:1;2384/chr14:3
97:chr14:38091610-38091436:2;2385/chr14:397:chr14:38091610-38091436:1;2386/chr1:3867:chr1:45082905-4
5082774:2;2387/chr1:3867:chr1:45082905-45082774:1;2388/chr14:2833:chr14:106320742-106320572:2;2389/
chr11:2506:chr11:65408750-65408579:2;2390/chr13:518:chr13:37005966-37005791:2;2391/chr13:518:chr13:3
7005966-37005791:1;2392/chr17:3474:chr17:73584759-73584589:2;2393/chr1:4957:chr1:115632197-1156320
25:2;2394/chr1:4957:chr1:115632197-115632025:1;2395/chr5:839:chr5:3602462-3602337:2;2396/chr5:839:chr
5:3602462-3602337:1;2397/chrX:503:chrX:21676497-21676339:2;2398/chrX:503:chrX:21676497-21676339:1;2
399/chr9:1070:chr9:96714415-96714285:2;2400/chr9:1070:chr9:96714415-96714285:1;2401/chr1:3916:chr1:46
951712-46951543:2;2402/chr1:3916:chr1:46951712-46951543:1;2403/chr17:2307:chr17:45786630-45786466:2;
2404/chr17:2307:chr17:45786630-45786466:1;2405/chr10:2874:chr10:105344832-105344671:2;2406/chr10:28
74:chr10:105344832-105344671:1;2407/chr14:688:chr14:61747024-61746883:2;2408/chr14:688:chr14:617470
24-61746883:1;2409/chr16:3863:chr16:86963006-86962832:1;2410/chr17:1933:chr17:38334321-38334151:2;2
411/chr13:1687:chr13:113699564-113699389:2;2412/chr15:1556:chr15:76638648-76638475:2;2413/chr8:1101:
chr8:23584680-23584524:2;2414/chr18:763:chr18:44336779-44336618:2;2415/chr18:763:chr18:44336779-443
36618:1;2416/chr12:1118:chr12:52115685-52115513:2;2417/chr12:1118:chr12:52115685-52115513:1;2418/chr
4:2398:chr4:135122848-135122677:2;2419/cg27547954:chr1:76082092-76081923:1;2420/chr12:1296:chr12:54
355667-54355516:1;2421/chr17:4148:chr17:77814286-77814117:2;2422/chr17:4148:chr17:77814286-7781411
7:1;2423/chr19:2231:chr19:18980885-18980714:2;2424/chr19:2231:chr19:18980885-18980714:1;2425/chr4:23
98:chr4:135122848-135122677:1;2426/chr5:2325:chr5:134364525-134364359:2;2427/chr5:2325:chr5:1343645
25-134364359:1;2428/chr22:163:chr22:19711924-19711764:2;2429/chr22:163:chr22:19711924-19711764:1;24
30/chr3:2978:chr3:170137582-170137429:2;2431/chr3:2978:chr3:170137582-170137429:1;2432/chr4:2647:chr
4:164264966-164264821:2;2433/chr4:2647:chr4:164264966-164264821:1;2434/chr1:3845:chr1:44704172-4470
3998:2;2435/chr1:3845:chr1:44704172-44703998:1;2436/chr1:4624:chr1:91183323-91183196:2;2437/chr8:174
4:chr8:55382938-55382776:2;2438/chr8:1744:chr8:55382938-55382776:1;2439/chr3:2897:chr3:157821637-157
821493:2;2440/chr3:2897:chr3:157821637-157821493:1;2441/chr7:4153:chr7:150812229-150812057:2;2442/c
hr7:4153:chr7:150812229-150812057:1;2443/chr19:3497:chr19:41106377-41106210:2;2444/chr19:3497:chr19:
41106377-41106210:1;2445/chr13:1060:chr13:100608190-100608021:2;2446/chr3:220:chr3:10857967-108578
26:2;2447/chr3:220:chr3:10857967-10857826:1;2448/chr15:1086:chr15:65116160-65116014:2;2449/chr15:108
6:chr15:65116160-65116014:1;2450/chr8:1744:chr8:55383226-55383051:2;2451/chr8:1744:chr8:55383226-553
83051:1;2452/chr8:1350:chr8:37556046-37555873:2;2453/chr2:4122:chr2:172948139-172947964:2;2454/chr2:
4122:chr2:172948139-172947964:1;2455/chr18:637:chr18:35065277-35065105:2;2456/chr10:4449:chr10:1339
99214-133999086:2;2457/chr10:4449:chr10:133999214-133999086:1;2458/chr20:925:chr20:32256030-322559
05:2;2459/chr20:925:chr20:32256030-32255905:1;2460/chr7:811:chr7:2968367-2968192:2;2461/chr7:811:chr7:
2968367-2968192:1;2462/chr1:4908:chr1:113052067-113051902:2;2463/chr7:2148:chr7:49815450-49815286:2;
2464/chr7:2148:chr7:49815450-49815286:1;2465/chr9:1343:chr9:110250594-110250466:2;2466/chr9:1343:chr
9:110250594-110250466:1;2467/chr1:4908:chr1:113052067-113051902:1;2468/chr20:2928:chr20:62119971-62
119827:2;2469/chr20:2928:chr20:62119971-62119827:1;2470/chr18:637:chr18:35065277-35065105:1;2471/chr
19:3278:chr19:38886717-38886583:2;2472/chr19:3278:chr19:38886717-38886583:1;2473/chr10:2794:chr10:10
3539053-103538883:2;2474/chr10:2794:chr10:103539053-103538883:1;2475/chr10:2898:chr10:106028659-10
6028486:2;2476/chr14:157:chr14:24780839-24780665:2;2477/chr14:157:chr14:24780839-24780665:1;2478/chr
7:4786:chr7:157406423-157406254:2;2479/chr19:2540:chr19:30017380-30017217:2;2480/chr19:2540:chr19:30
017380-30017217:1;2481/chr19:1722:chr19:13616996-13616867:2;2482/chr19:1722:chr19:13616996-1361686
7:1;2483/chr2:259:chr2:1748402-1748230:2;2484/chr2:259:chr2:1748402-1748230:1;2485/chr18:1044:chr18:56
940406-56940232:2;2486/chr18:1044:chr18:56940406-56940232:1;2487/chr17:2175:chr17:43045036-4304486
1:2;2488/chr15:1086:chr15:65116218-65116090:2;2489/chr15:1086:chr15:65116218-65116090:1;2490/chr5:82
7:chr5:3594626-3594465:2;2491/chr5:827:chr5:3594626-3594465:1;2492/chr7:2143:chr7:49813614-49813457:
2;2493/chr7:2143:chr7:49813614-49813457:1;2494/chr1:254:chr1:1566696-1566533:2;2495/chr1:254:chr1:156
6696-1566533:1;2496/chr12:787:chr12:33593020-33592846:2;2497/chr12:787:chr12:33593020-33592846:1;24
98/chr20:752:chr20:25565828-25565656:2;2499/chr20:752:chr20:25565828-25565656:1;2500/chr19:369:chr19:
1470324-1470155:2;2501/chr19:4944:chr19:58908209-58908046:2;2502/chr19:4944:chr19:58908209-5890804
6:1;2503/chr19:553:chr19:2252403-2252228:2;2504/chr19:553:chr19:2252403-2252228:1;2505/chr10:1956:chr
10:73847427-73847253:2;2506/chr10:1956:chr10:73847427-73847253:1;2507/chr8:728:chr8:11538082-115379
54:2;2508/chr8:728:chr8:11538082-11537954:1;2509/chr19:2193:chr19:18771306-18771161:2;2510/chr6:138:c
hr6:1615052-1614927:2;2511/chr9:2530:chr9:136293676-136293533:2;2512/chr9:2530:chr9:136293676-13629
3533:1;2513/chr2:5420:chr2:239755961-239755791:2;2514/chr2:5420:chr2:239755961-239755791:1;2515/chr6:
138:chr6:1615052-1614927:1;2516/chr16:110:chr16:744330-744162:2;2517/chr16:110:chr16:744330-744162:1;
2518/chr17:309:chr17:2627897-2627740:2;2519/chr17:309:chr17:2627897-2627740:1;2520/chr13:1060:chr13:
100608540-100608369:2;2521/chr13:1060:chr13:100608540-100608369:1;2522/chr11:2506:chr11:65408580-6
5408406:2;2523/chr11:2506:chr11:65408580-65408406:1;2524/chr18:891:chr18:47794997-47794822:1;2525/c
hr1:4372:chr1:63790019-63789889:2;2526/chr1:4372:chr1:63790019-63789889:1;2527/chr15:1200:chr15:6812
0646-68120487:2;2528/chr15:1200:chr15:68120646-68120487:1;2529/chr9:1188:chr9:99482349-99482174:2;2
530/chr9:1188:chr9:99482349-99482174:1;2531/chr8:1350:chr8:37555990-37555815:2;2532/chr8:1350:chr8:37
555990-37555815:1;2533/chr6:2263:chr6:108489895-108489726:2;2534/chr1:5286:chr1:154475206-15447503
1:2;2535/chr10:2027:chr10:77159083-77158919:2;2536/chr10:2027:chr10:77159083-77158919:1;2537/chr1:52
86:chr1:154475206-154475031:1;2538/chr5:2048:chr5:112539112-112538947:2;2539/chr5:2048:chr5:1125391
12-112538947:1;2540/chr15:753:chr15:45479590-45479420:2;2541/chr15:753:chr15:45479590-45479420:1;25
42/chr16:757:chr16:3017558-3017426:1;2543/chr2:4183:chr2:175200640-175200506:2;2544/chr2:4183:chr2:17
5200640-175200506:1;2545/chr9:2530:chr9:136293794-136293619:2;2546/chr9:2530:chr9:136293794-136293
619:1;2547/chr21:1265:chr21:47062423-47062264:2;2548/chr21:1265:chr21:47062423-47062264:1;2549/chr2:
1376:chr2:29033898-29033723:2;2550/chr2:1376:chr2:29033898-29033723:1;2551/chr12:428:chr12:6664942-6
664801:2;2552/chr12:428:chr12:6664942-6664801:1;2553/chr19:3873:chr19:46997239-46997102:2;2554/chr19:
3873:chr19:46997239-46997102:1;2555/chr11:117:chr11:725876-725704:2;2556/chr11:117:chr11:725876-725
704:1;2557/chr10:4752:chr10:134862411-134862284:2;2558/cg15674193:chr2:238536007-238535834:2;2559/
cg15674193:chr2:238536007-238535834:1;2560/chr16:4191:chr16:88701134-88701002:2;2561/chr16:4191:chr
16:88701134-88701002:1;2562/chr14:1137:chr14:77492380-77492248:2;2563/chr14:1137:chr14:77492380-774
92248:1;2564/chr10:4752:chr10:134862411-134862284:1;2565/chr8:2284:chr8:102506107-102505949:2;2566/
chr8:2284:chr8:102506107-102505949:1;2567/chr5:1055:chr5:7850071-7849896:2;2568/chr5:1055:chr5:78500
71-7849896:1;2569/chr5:1850:chr5:79331278-79331104:2;2570/chr5:1850:chr5:79331278-79331104:1;2571/ch
r1:4827:chr1:110613397-110613272:2;2572/chr1:4827:chr1:110613397-110613272:1;2573/chr19:3833:chr19:4
6443496-46443322:2;2574/chr19:3833:chr19:46443496-46443322:1;2575/chr17:4511:chr17:79615448-796153
21:2;2576/chr17:4511:chr17:79615448-79615321:1;2577/chr7:1376:chr7:23245651-23245480:2;2578/chr7:137
6:chr7:23245651-23245480:1;2579/chr15:264:chr15:28352821-28352655:2;2580/chr15:264:chr15:28352821-28
352655:1;2581/chr10:2350:chr10:88728518-88728344:2;2582/chr10:2350:chr10:88728518-88728344:1;2583/c
hr17:2799:chr17:59474379-59474254:2;2584/chr15:2015:chr15:89952706-89952533:2;2585/chr15:2015:chr15:
89952706-89952533:1;2586/chr17:2799:chr17:59474379-59474254:1;2587/chr2:1264:chr2:26408202-2640802
9:2;2588/chr2:1264:chr2:26408202-26408029:1;2589/chr8:651:chr8:10588041-10587910:2;2590/chr8:651:chr8:
10588041-10587910:1;2591/chr5:1410:chr5:40682164-40681990:2;2592/chr5:1410:chr5:40682164-40681990:1;
2593/chr14:354:chr14:37127981-37127809:1;2594/chr16:2526:chr16:55543217-55543042:2;2595/chr16:2526:
chr16:55543217-55543042:1;2596/chr1:2371:chr1:16085782-16085615:2;2597/chr1:2371:chr1:16085782-1608
5615:1;2598/chr10:1049:chr10:30024428-30024286:2;2599/chr10:1049:chr10:30024428-30024286:1;2600/chr1
5:203:chr15:27018841-27018713:2;2601/chr15:203:chr15:27018841-27018713:1;2602/chr21:861:chr21:455779
24-45577757:2;2603/chr21:861:chr21:45577924-45577757:1;2604/chr6:2384:chr6:117086916-117086743:2;26
05/chr6:2384:chr6:117086916-117086743:1;2606/chr19:369:chr19:1470324-1470155:1;2607/chr19:392:chr19:1
524198-1524069:2;2608/chr19:392:chr19:1524198-1524069:1;2609/chr19:2193:chr19:18771306-18771161:1;2
610/chr21:333:chr21:38081403-38081231:2;2611/chr21:333:chr21:38081403-38081231:1;2612/chr12:464:chr1
2:7592832-7592706:2;2613/chr12:464:chr12:7592832-7592706:1;2614/chr6:2263:chr6:108489895-108489726:
1;2615/chr2:1519:chr2:39187419-39187294:2;2616/chr2:1519:chr2:39187419-39187294:1;2617/chr2:2631:chr2:
99439369-99439201:2;2618/chr2:2631:chr2:99439369-99439201:1;2619/chr8:728:chr8:11537844-11537669:2;
2620/chr8:728:chr8:11537844-11537669:1;2621/chr7:1552:chr7:27225264-27225111:2;2622/chr7:1552:chr7:27
225264-27225111:1;2623/chr20:764:chr20:26189263-26189094:2;2624/chr20:764:chr20:26189263-26189094:1;
2625/chr14:2165:chr14:103395785-103395613:2;2626/chr14:2165:chr14:103395785-103395613:1;2627/chr2:4
214:chr2:176945075-176944913:2;2628/chr2:4214:chr2:176945075-176944913:1;2629/chr8:3702:chr8:145105
760-145105618:2;2630/chr8:3702:chr8:145105760-145105618:1;2631/chr10:4868:chr10:135015757-13501558
4:2;2632/chr10:4868:chr10:135015757-135015584:1;2633/chr2:4218:chr2:176948297-176948168:2;2634/chr2:
4218:chr2:176948297-176948168:1;2635/chr11:3710:chr11:100999084-100998915:2;2636/chr11:3710:chr11:1
00999084-100998915:1;2637/chr17:279:chr17:1962683-1962534:1;2638/chr4:1604:chr4:37247113-37246974:2;
2639/chr4:1604:chr4:37247113-37246974:1;2640/chr8:2181:chr8:97158066-97157894:2;2641/chr8:2181:chr8:
97158066-97157894:1;2642/chr7:1514:chr7:27187677-27187502:2;2643/chr7:1514:chr7:27187677-27187502:1;
2644/chr8:1101:chr8:23584680-23584524:1;2645/chr17:1243:chr17:17626082-17625913:2;2646/chr17:1243:c
hr17:17626082-17625913:1;2647/chr7:3315:chr7:103630346-103630184:2;2648/chr7:3315:chr7:103630346-10
3630184:1;2649/chr20:62:chr20:982972-982798:2;2650/chr20:62:chr20:982972-982798:1;2651/chr3:392:chr3:1
3324257-13324082:2;2652/chr3:392:chr3:13324257-13324082:1;2653/chr13:1456:chr13:112721494-11272132
0:2;2654/chr13:1456:chr13:112721494-112721320:1;2655/chr1:3916:chr1:46951428-46951253:2;2656/chr1:39
16:chr1:46951428-46951253:1;2657/chr6:2032:chr6:88876633-88876458:2;2658/chr6:2032:chr6:88876633-888
76458:1;2659/chr5:91:chr5:528704-528579:2;2660/chr5:91:chr5:528704-528579:1;2661/chr16:3825:chr16:8654
4686-86544555:2;2662/chr16:3825:chr16:86544686-86544555:1;2663/chr14:2041:chr14:101925746-10192557
1:2;2664/chr14:2041:chr14:101925746-101925571:1;2665/chr1:4624:chr1:91183323-91183196:1;2666/chr13:1
060:chr13:100608190-100608021:1;2667/chr5:2254:chr5:132161425-132161269:2;2668/chr5:2254:chr5:13216
1425-132161269:1;2669/chr9:431:chr9:37002824-37002679:2;2670/chr9:431:chr9:37002824-37002679:1;2671/
chr3:20:chr3:2140592-2140466:2;2672/chr3:20:chr3:2140592-2140466:1;2673/chr8:1350:chr8:37556046-37555
873:1;2674/chr1:4371:chr1:63789575-63789403:2;2675/chr1:4371:chr1:63789575-63789403:1;2676/chr9:3497:
chr9:139716502-139716342:2;2677/chr9:3497:chr9:139716502-139716342:1;2678/chr2:4262:chr2:176995166-
176995024:2;2679/chr2:4262:chr2:176995166-176995024:1;2680/chr4:1277:chr4:8873515-8873346:2;2681/chr
4:1277:chr4:8873515-8873346:1;2682/chr9:1715:chr9:126780232-126780103:2;2683/chr9:1715:chr9:12678023
2-126780103:1;2684/chr8:3701:chr8:145104288-145104163:2;2685/chr8:3701:chr8:145104288-145104163:1;2
686/chr10:2898:chr10:106028659-106028486:1;2687/chr22:2727:chr22:50738621-50738496:2;2688/chr22:272
7:chr22:50738621-50738496:1;2689/chr7:4786:chr7:157406423-157406254:1;2690/chr10:4942:chr10:1351933
40-135193215:2;2691/chr10:4942:chr10:135193340-135193215:1;2692/chr17:2175:chr17:43045036-43044861:
1;2693/chr22:34:chr22:17566751-17566576:2;2694/chr22:34:chr22:17566751-17566576:1;2695/chr22:2146:chr
22:48885020-48884883:2;2696/chr22:2146:chr22:48885020-48884883:1;2697/chr10:3443:chr10:124894879-12
4894706:2;2698/chr10:3443:chr10:124894879-124894706:1;2699/chr20:2966:chr20:62272772-62272625:2;270
0/chr1:1974:chr1:10698909-10698748:2;2701/chr1:1974:chr1:10698909-10698748:1;2702/chr12:1858:chr12:10
4609873-104609704:2;2703/chr12:1858:chr12:104609873-104609704:1;2704/chr14:578:chr14:57265030-5726
4866:2;2705/chr14:578:chr14:57265030-57264866:1;2706/chr20:2966:chr20:62272772-62272625:1;2707/chr10:
4898:chr10:135052102-135051929:2;2708/chr10:4898:chr10:135052102-135051929:1;2709/chr13:865:chr13:7
9170284-79170114:2;2710/chr13:865:chr13:79170284-79170114:1;2711/chr8:1716:chr8:54789950-54789795:2;
2712/chr8:1716:chr8:54789950-54789795:1;2713/chr9:1173:chr9:98981719-98981552:2;2714/chr9:1173:chr9:
98981719-98981552:1;2715/chr3:2880:chr3:156544188-156544017:2;2716/chr3:2880:chr3:156544188-156544
017:1;2717/chr17:4148:chr17:77814525-77814362:2;2718/chr17:4148:chr17:77814525-77814362:1;2719/chr15:
203:chr15:27018478-27018349:2;2720/chr15:203:chr15:27018478-27018349:1;2721/chr5:2560:chr5:14087212
7-140871960:2;2722/chr5:2560:chr5:140872127-140871960:1;2723/chr2:5386:chr2:239072749-239072583:2;2
724/chr2:5386:chr2:239072749-239072583:1;2725/chr2:4451:chr2:200328795-200328620:2;2726/chr2:4451:ch
r2:200328795-200328620:1;2727/chr10:4270:chr10:133110422-133110289:2;2728/chr10:4270:chr10:13311042
2-133110289:1;2729/chr17:537:chr17:4804494-4804320:2;2730/chr17:537:chr17:4804494-4804320:1;2731/chr
4:1270:chr4:8863083-8862930:2;2732/chr3:1095:chr3:47051232-47051073:2;2733/chr3:1095:chr3:47051232-4
7051073:1;2734/chr18:431:chr18:19751703-19751533:2;2735/chr18:431:chr18:19751703-19751533:1;2736/chr
14:2727:chr14:105714683-105714520:2;2737/chr14:2727:chr14:105714683-105714520:1;2738/chr10:2698:chr
10:102586654-102586485:2;2739/chr10:2698:chr10:102586654-102586485:1;2740/chr4:1270:chr4:8863083-88
62930:1;2741/chr16:4179:chr16:88600968-88600831:2;2742/chr16:4179:chr16:88600968-88600831:1;2743/chr
8:13:chr8:494614-494452:2;2744/chr8:13:chr8:494614-494452:1;2745/chr16:4071:chr16:88443212-88443081:2;
2746/chr16:4071:chr16:88443212-88443081:1;2747/chr15:1556:chr15:76638940-76638814:2;2748/chr15:1556:
chr15:76638940-76638814:1;2749/chr22:2205:chr22:48971392-48971255:2;2750/chr22:2205:chr22:48971392-
48971255:1;2751/chr10:950:chr10:26223430-26223266:2;2752/chr10:950:chr10:26223430-26223266:1;2753/c
hr9:3297:chr9:139094816-139094645:2;2754/chr9:3297:chr9:139094816-139094645:1;2755/chr2:2240:chr2:75
147174-75147005:2;2756/chr2:2240:chr2:75147174-75147005:1;2757/chr5:2450:chr5:138729982-138729843:2;
2758/chr13:1076:chr13:100642220-100642086:2;2759/chr13:1076:chr13:100642220-100642086:1;2760/chr4:2
4:chr4:568940-568773:2;2761/chr4:24:chr4:568940-568773:1;2762/chr1:2205:chr1:12123634-12123491:2;2763/
chr1:2205:chr1:12123634-12123491:1;2764/chr14:986:chr14:73958188-73958044:2;2765/chr14:986:chr14:739
58188-73958044:1;2766/cg17300544:chr17:75369111-75368945:2;2767/cg17300544:chr17:75369111-753689
45:1;2768/chr12:428:chr12:6665108-6664937:2;2769/chr8:38:chr8:687589-687464:2;2770/chr8:38:chr8:687589-
687464:1;2771/chr9:631:chr9:79638047-79637875:2;2772/chr9:631:chr9:79638047-79637875:1;2773/chr10:24
07:chr10:94451766-94451592:2;2774/chr10:2407:chr10:94451766-94451592:1;2775/chr16:2386:chr16:511855
72-51185406:2;2776/chr16:2386:chr16:51185572-51185406:1;2777/chr19:367:chr19:1468844-1468700:2;2778/
chr19:367:chr19:1468844-1468700:1;2779/chr16:234:chr16:1035246-1035107:2;2780/chr16:234:chr16:103524
6-1035107:1;2781/chr5:2450:chr5:138729982-138729843:1;2782/chr11:389:chr11:1578344-1578214:2;2783/ch
r11:389:chr11:1578344-1578214:1;2784/chr1:3957:chr1:47904779-47904638:2;2785/chr1:3957:chr1:47904779-
47904638:1;2786/chr16:110:chr16:744266-744099:2;2787/chr16:110:chr16:744266-744099:1;2788/chr17:1606:
chr17:30244359-30244184:2;2789/chr17:1606:chr17:30244359-30244184:1;2790/chr5:2729:chr5:149546999-1
49546827:2;2791/chr5:2729:chr5:149546999-149546827:1;2792/chr19:452:chr19:1762666-1762493:2;2793/chr
19:452:chr19:1762666-1762493:1;2794/chr9:1708:chr9:126778026-126777897:1;2795/chr7:1377:chr7:2328762
8-23287486:2;2796/chr7:1377:chr7:23287628-23287486:1;2797/chr2:1550:chr2:42328826-42328653:2;2798/ch
r2:1550:chr2:42328826-42328653:1;2799/chr2:3925:chr2:154729086-154728912:2;2800/chr2:3925:chr2:15472
9086-154728912:1;2801/chr22:1367:chr22:42679830-42679656:2;2802/chr22:1367:chr22:42679830-42679656:
1;2803/chr2:2227:chr2:74743505-74743333:2;2804/chr2:2227:chr2:74743505-74743333:1;2805/chr2:2750:chr2:
105459706-105459542:2;2806/chr2:2750:chr2:105459706-105459542:1;2807/chr16:375:chr16:1203225-12031
00:2;2808/chr16:375:chr16:1203225-1203100:1;2809/chr17:1078:chr17:12877677-12877539:2;2810/chr17:107
8:chr17:12877677-12877539:1;2811/chr3:394:chr3:13324791-13324621:2;2812/chr3:394:chr3:13324791-13324
621:1;2813/chr5:3133:chr5:170878110-170877975:2;2814/chr5:3133:chr5:170878110-170877975:1;2815/chr7:
405:chr7:1276434-1276303:2;2816/chr7:405:chr7:1276434-1276303:1;2817/chr20:1816:chr20:55206152-55206
015:2;2818/chr20:1816:chr20:55206152-55206015:1;2819/chr17:965:chr17:10102204-10102079:2;2820/chr17:
965:chr17:10102204-10102079:1;2821/chr10:2022:chr10:77155991-77155834:2;2822/chr10:2022:chr10:77155
991-77155834:1;2823/chr10:4581:chr10:134598258-134598089:2;2824/chr10:4581:chr10:134598258-1345980
89:1;2825/chr13:401:chr13:29394321-29394149:2;2826/chr13:401:chr13:29394321-29394149:1;2827/chr8:100
6:chr8:22408833-22408672:2;2828/chr8:1006:chr8:22408833-22408672:1;2829/chr9:55:chr9:1045795-1045621:
1;2830/chr22:1921:chr22:46932455-46932290:1;2831/chr11:3716:chr11:101454561-101454393:2;2832/chr11:
937:chr11:2871857-2871682:2;2833/chr11:937:chr11:2871857-2871682:1;2834/chr5:91:chr5:528659-528501:2;
2835/chr5:91:chr5:528659-528501:1;2836/chr11:3716:chr11:101454561-101454393:1;2837/chr5:1864:chr5:802
56353-80256211:2;2838/chr5:1864:chr5:80256353-80256211:1;2839/chr7:562:chr7:1626073-1625900:2;2840/c
hr7:562:chr7:1626073-1625900:1;2841/chr1:222:chr1:1475323-1475176:2;2842/chr1:222:chr1:1475323-14751
76:1;2843/chr22:2205:chr22:48971239-48971108:2;2844/chr22:2205:chr22:48971239-48971108:1;2845/chr20:
1878:chr20:55965620-55965464:1;2846/chr12:1757:chr12:95942371-95942241:2;2847/chr12:1757:chr12:9594
2371-95942241:1;2848/chr8:650:chr8:10587774-10587649:2;2849/chr8:650:chr8:10587774-10587649:1;2850/c
hr16:393:chr16:1211002-1210828:1;2851/chr8:3309:chr8:143545744-143545576:1;2852/chr10:4269:chr10:133
109593-133109429:2;2853/chr10:4269:chr10:133109593-133109429:1;2854/chrX:537:chrX:25020769-2502059
6:2;2855/chrX:537:chrX:25020769-25020596:1;2856/chr22:2205:chr22:48971617-48971472:2;2857/chr22:2205:
chr22:48971617-48971472:1;2858/chr5:2942:chr5:159399401-159399233:2;2859/chr5:2942:chr5:159399401-1
59399233:1;2860/chr18:1140:chr18:67068423-67068255:2;2861/chr18:1140:chr18:67068423-67068255:1;2862/
chr10:3746:chr10:129537074-129536938:2;2863/chr10:3746:chr10:129537074-129536938:1;2864/chr11:2301:
chr11:63767899-63767753:2;2865/chr11:2301:chr11:63767899-63767753:1;2866/chr1:1852:chr1:8277493-827
7353:2;2867/chr1:1852:chr1:8277493-8277353:1;2868/chr15:1842:chr15:84323046-84322896:2;2869/chr15:18
42:chr15:84323046-84322896:1;2870/chr12:1322:chr12:54441309-54441134:2;2871/chr18:487:chr18:2292984
4-22929696:1;2872/chr7:1085:chr7:49233314923169:2;2873/chr7:1085:chr7:49233314923169:1;2874/chr10:2
949:chr10:108924567-108924400:1;2875/chr19:1881:chr19:15580502-15580347:2;2876/chr19:1881:chr19:155
80502-15580347:1;2877/chr7:4544:chr7:155595962-155595836:2;2878/chr7:4544:chr7:155595962-155595836:
1;2879/chr8:1101:chr8:23584583-23584456:2;2880/chr8:1101:chr8:23584583-23584456:1;2881/chr16:241:chr1
6:1041428-1041270:2;2882/chr16:241:chr16:1041428-1041270:1;2883/chr2:2240:chr2:75147237-75147063:2;2
884/chr2:2240:chr2:75147237-75147063:1;2885/chr7:3616:chr7:130418792-130418660:2;2886/chr7:3616:chr7:
130418792-130418660:1;2887/chr18:891:chr18:4779475747794617:2;2888/chr18:891:chr18:4779475747794
617:1;2889/chr8:2051:chr8:80804177-80804024:1;2890/chr5:2253:chr5:132159004-132158842:2;2891/chr5:22
53:chr5:132159004-132158842:1;2892/chr12:1322:chr12:54441309-54441134:1;2893/chr4:2610:chr4:1556652
05-155665030:2;2894/chr4:2610:chr4:155665205-155665030:1;2895/chr15:2502:chr15:101514058-101513916:
2;2896/chr15:2502:chr15:101514058-101513916:1;2897/chr18:966:chr18:53447556-53447382:2;2898/chr18:96
6:chr18:53447556-53447382:1;2899/chr14:2327:chr14:104622032-104621858:2;2900/chr14:2327:chr14:10462
2032-104621858:1;2901/chr13:1471:chr13:112758838-112758663:2;2902/chr13:1471:chr13:112758838-11275
8663:1;2903/chr8:38:chr8:687325-687194:2;2904/chr8:38:chr8:687325-687194:1;2905/chr16:2445:chr16:54324
557-54324430:1;2906/chr5:830:chr5:3596431-3596260:2;2907/chr5:830:chr5:3596431-3596260:1;2908/chr14:2
099:chr14:102172468-102172295:2;2909/chr14:2099:chr14:102172468-102172295:1;2910/chr14:2833:chr14:1
06320742-106320572:1;2911/chr11:2506:chr11:65408750-65408579:1;2912/chr17:3474:chr17:73584759-7358
4589:1;2913/chr18:1613:chr18:76740998-76740823:2;2914/chr18:1613:chr18:76740998-76740823:1;2915/chr4:
1747:chr4:48485614-48485443:2;2916/chr4:1747:chr4:48485614-48485443:1;2917/chr8:2251:chr8:99986089-
99985945:2;2918/chr8:2251:chr8:99986089-99985945:1;2919/chr17:1933:chr17:38334321-38334151:1;2920/c
hr13:1687:chr13:113699564-113699389:1;2921/chr12:1613:chr12:72666105-72665937:2;2922/chr15:1556:chr1
5:76638648-76638475:1;2923/chr12:1613:chr12:72666105-72665937:1;2924/chr19:550:chr19:2250948-225081
5:2;2925/chr19:550:chr19:2250948-2250815:1;2926/chr13:236:chr13:25320287-25320147:2;2927/chr13:236:ch
r13:25320287-25320147:1;2928/chr17:176:chr17:1173964-1173804:2;2929/chr17:176:chr17:1173964-1173804:
1;2930/chr22:161:chr22:19711392-19711223:1;2931/chr17:4140:chr17:77789918-77789750:2;2932/chr17:414
0:chr17:77789918-77789750:1;2933/chr2:1376:chr2:29033996-29033861:2;2934/chr2:1376:chr2:29033996-290
33861:1;2935/chr10:2308:chr10:88123432-88123257:2;2936/chr10:2308:chr10:88123432-88123257:1;2937/chr
12:3619:chr12:133485057-133484892:2;2938/chr12:3619:chr12:133485057-133484892:1;2939/chr22:2149:chr
22:4888617548886001:2;2940/chr22:2149:chr22:4888617548886001:1;2941/chrX:396:chrX:12156996-12156
849:1;2942/chr14:2833:chr14:106320894-106320722:1;2943/chr14:1314:chr14:90528870-90528732:2;2944/chr
14:1314:chr14:90528870-90528732:1;2945/chr17:3421:chr17:73073968-73073797:2;2946/chr17:3421:chr17:73
073968-73073797:1;2947/chr6:95:chr6:1379007-1378882:2;2948/chr6:95:chr6:1379007-1378882:1;2949/chr4:2
05:chr4:1400150-1399979:2;2950/chr4:205:chr4:1400150-1399979:1;2951/chr3:9:chr3:240192-240027:2;2952/
chr3:9:chr3:240192-240027:1;2953/chr16:234:chr16:1035536-1035395:1;2954/chr11:3716:chr11:101454331-10
1454156:2;2955/chr11:3716:chr11:101454331-101454156:1;2956/chr1:1191:chr1:3460405-3460237:2;2957/chr1:
1191:chr1:3460405-3460237:1;2958/chr6:2499:chr6:133562585-133562411:2;2959/chr10:808:chr10:1842948
5-18429326:2;2960/chr10:808:chr10:18429485-18429326:1;2961/chr6:2499:chr6:133562585-133562411:1;296
2/chr1:59:chr1:969792-969664:1;2963/chr5:2324:chr5:134364182-134364038:2;2964/chr5:2324:chr5:13436418
2-134364038:1;2965/chr15:982:chr15:60296694-60296550:1;2966/chr2:5386:chr2:239073108-239072982:2;29
67/chr2:5386:chr2:239073108-239072982:1;2968/chr9:1218:chr9:100611190-100611020:2;2969/chr9:1218:chr
9:100611190-100611020:1;2970/chr10:519:chr10:8094175-8094000:2;2971/chr10:519:chr10:8094175-8094000:
1;2972/chr16:2668:chr16:58059026-58058856:2;2973/chr16:2668:chr16:58059026-58058856:1;2974/chr12:51:
chr12:680038-679865:1;2975/chr14:881:chr14:70038850-70038715:2;2976/chr14:881:chr14:70038850-700387
15:1;2977/chr18:1054:chr18:57364312-57364187:2;2978/chr18:1054:chr18:57364312-57364187:1;2979/chr15:
1538:chr15:76630392-76630241:2;2980/chr15:1538:chr15:76630392-76630241:1;2981/chr17:534:chr17:48023
79-4802211:2;2982/chr17:534:chr17:4802379-4802211:1;2983/chr10:1956:chr10:73847147-73846977:2;2984/c
hr10:1956:chr10:73847147-73846977:1;2985/chr10:983:chr10:28034670-28034501:2;2986/chr10:983:chr10:28
034670-28034501:1;2987/chr14:1032:chr14:75078676-75078512:2;2988/chr14:1032:chr14:75078676-7507851
2:1;2989/cg23010538:chr1:190447363-190447191:2;2990/cg23010538:chr1:190447363-190447191:1;2991/chr
16:3215:chr16:77469048-77468873:2;2992/chr16:3215:chr16:77469048-77468873:1;2993/chr20:90:chr20:1784
553-1784408:2;2994/chr20:90:chr20:1784553-1784408:1;2995/chr8:1276:chr8:31498326-31498151:2;2996/chr
8:1276:chr8:31498326-31498151:1;2997/chr9:1878:chr9:129377646-129377494:2;2998/chr9:1878:chr9:129377
646-129377494:1;2999/chr15:937:chr15:58358265-58358124:2;3000/chr15:937:chr15:58358265-58358124:1;3
001/chr7:1581:chr7:27291354-27291188:2;3002/chr7:1581:chr7:27291354-27291188:1;3003/chr2:599:chr2:583
3113-5832943:2;3004/chr2:599:chr2:5833113-5832943:1;3005/chr14:881:chr14:70038726-70038555:2;3006/ch
r14:881:chr14:70038726-70038555:1;3007/chr19:298:chr19:1257045-1256913:1;3008/chr2:4182:chr2:1751999
41-175199771:2;3009/chr2:4182:chr2:175199941-175199771:1;3010/chr1:4372:chr1:63790172-63790002:2;30
11/chr1:4372:chr1:63790172-63790002:1;3012/chr6:279:chr6:6004155-6003980:2;3013/chr4:1750:chr4:484932
58-48493088:2;3014/chr4:241:chr4:1538150-1537977:2;3015/chr2:4187:chr2:175201796-175201640:2;3016/ch
r2:4187:chr2:175201796-175201640:1;3017/chr8:652:chr8:10588501-10588328:2;3018/chr8:652:chr8:1058850
1-10588328:1;3019/chr16:2907:chr16:67687653-67687484:2;3020/chr19:367:chr19:1468705-1468541:2;3021/c
hr19:367:chr19:1468705-1468541:1;3022/chr17:2816:chr17:59488492-59488354:2;3023/chr17:2816:chr17:594
88492-59488354:1;3024/chr16:2907:chr16:67687653-67687484:1;3025/chr12:1118:chr12:52115740-52115566:
2;3026/chr12:1118:chr12:52115740-52115566:1;3027/chr10:4059:chr10:131763726-131763587:2;3028/chr10:4
059:chr10:131763726-131763587:1;3029/chr5:1453:chr5:43397365-43397230:1;3030/chr5:2252:chr5:1321502
64-132150092:2;3031/chr5:2252:chr5:132150264-132150092:1;3032/chr3:2636:chr3:138664513-138664353:2;
3033/chr3:2636:chr3:138664513-138664353:1;3034/chr4:241:chr4:1538150-1537977:1;3035/chr21:1265:chr21:
47062332-47062163:2;3036/chr21:1265:chr21:47062332-47062163:1;3037/chr12:1462:chr12:58131504-58131
379:2;3038/chr12:1462:chr12:58131504-58131379:1;3039/chr14:393:chr14:38067764-38067595:2;3040/chr13:
518:chr13:37005849-37005704:2;3041/chr14:393:chr14:38067764-38067595:1;3042/chr13:518:chr13:3700584
9-37005704:1;3043/chr19:3621:chr19:42703922-42703777:2;3044/chr19:3621:chr19:42703922-42703777:1;30
45/chr15:260:chr15:28342442-28342269:2;3046/chr15:260:chr15:28342442-28342269:1;3047/chr12:2587:chr1
2:122016525-122016355:2;3048/chr12:2587:chr12:122016525-122016355:1;3049/chr5:2364:chr5:134871264-1
34871096:2;3050/chr5:2364:chr5:134871264-134871096:1;3051/chr5:3695:chr5:178422173-178422004:2;3052/
chr5:3695:chr5:178422173-178422004:1;3053/chr10:3445:chr10:124896574-124896438:2;3054/chr10:3445:ch
r10:124896574-124896438:1;3055/chr6:2384:chr6:117086979-117086804:2;3056/chr6:2384:chr6:117086979-1
17086804:1;3057/chr9:3295:chr9:139093813-139093640:2;3058/chr9:3295:chr9:139093813-139093640:1;3059/
chr12:1125:chr12:52215699-52215572:2;3060/chr12:1125:chr12:52215699-52215572:1;3061/chr1:5568:chr1:1
61008866-161008691:2;3062/chr4:1702:chr4:42153805-42153630:2;3063/chr4:1702:chr4:42153805-42153630:
1;3064/chr20:555:chr20:21503212-21503077:2;3065/chr20:555:chr20:21503212-21503077:1;3066/chr1:5568:c
hr1:161008866-161008691:1;3067/chr17:2514:chr17:48195201-48195027:2;3068/chr17:4566:chr17:79919700-
79919530:2;3069/chr17:4566:chr17:79919700-79919530:1;3070/chr17:2514:chr17:48195201-48195027:1;3071/
chr6:279:chr6:6004155-6003980:1;3072/chr4:1750:chr4:48493258-48493088:1;3073/chr15:219:chr15:2721669
3-27216557:2;3074/chr15:219:chr15:27216693-27216557:1;3075/chr4:2712:chr4:174428287-174428160:2;307
6/chr4:2712:chr4:174428287-174428160:1;3077/chr5:2246:chr5:132083549-132083377:2;3078/chr5:2246:chr5:
132083549-132083377:1;3079/chr10:2403:chr10:94450388-94450228:2;3080/chr10:2403:chr10:94450388-944
50228:1;3081/chr12:1296:chr12:54355355-54355224:2;3082/chr12:1296:chr12:54355355-54355224:1;3083/chr
10:2898:chr10:106028855-106028729:2;3084/chr10:2898:chr10:106028855-106028729:1;3085/chr3:2385:chr3:
128212350-128212177:2;3086/chr3:2385:chr3:128212350-128212177:1;3087/chr22:2759:chr22:50982929-509
82766:2;3088/chr22:2759:chr22:50982929-50982766:1;3089/chr19:3205:chr19:38042361-38042192:2;3090/chr
19:3205:chr19:38042361-38042192:1;3091/chr12:1457:chr12:58120333-58120208:2;3092/chr12:1457:chr12:58
120333-58120208:1;3093/chr7:1887:chr7:38670958-38670811:2;3094/chr7:1887:chr7:38670958-38670811:1;3
095/chr14:2041:chr14:101925149-101924999:2;3096/chr14:2041:chr14:101925149-101924999:1;3097/chr4:12
90:chr4:8895902-8895748:2;3098/chr4:1290:chr4:8895902-8895748:1;3099/chr14:688:chr14:61747361-617472
36:2;3100/chr14:688:chr14:61747361-61747236:1;3101/chr1:6573:chr1:217313129-217312955:1;3102/chr1:31
96:chr1:29101991-29101819:2;3103/chr1:3196:chr1:29101991-29101819:1;3104/chr2:4079:chr2:171570306-17
1570131:2;3105/chr2:4451:chr2:200328937-200328764:2;3106/chr5:511:chr5:1886181-1886013:2;3107/chr5:5
11:chr5:1886181-1886013:1;3108/chr9:629:chr9:79635846-79635721:2;3109/chr2:2224:chr2:74742325-747421
51:2;3110/chr1:253:chr1:1566083-1565912:2;3111/chr1:253:chr1:1566083-1565912:1;3112/chr9:1770:chr9:127
265719-127265594:2:3113/chr13:100:chr13:21296430-21296299:2;3114/chr13:100:chr13:21296430-21296299:
1;3115/chr13:708:chr13:53420276-53420109:2;3116/chr13:708:chr13:53420276-53420109:1;3117/chr2:2224:c
hr2:74742325-74742151:1;3118/chr9:1770:chr9:127265719-127265594:1;3119/chr6:2386:chr6:117198346-117
198180:2;3120/chr6:2386:chr6:117198346-117198180:1;3121/chr22:2764:chr22:50987137-50987002:2;3122/c
hr22:2764:chr22:50987137-50987002:1;3123/chr17:3789:chr17:76127596-76127460:2;3124/chr17:3789:chr17:
76127596-76127460:1;3125/chr22:2526:chr22:49969447-49969308:1;3126/chr21:1182:chr21:46847923-46847
798:2;3127/chr21:1182:chr21:46847923-46847798:1;3128/chr9:3185:chr9:138674930-138674795:2;3129/chr9:
3185:chr9:138674930-138674795:1;3130/chr22:2791:chr22:51135727-51135569:2;3131/chr22:2791:chr22:511
35727-51135569:1;3132/chr15:1843:chr15:84323496-84323323:2;3133/chr15:1843:chr15:84323496-84323323:
1;3134/chr11:4166:chr11:119211303-119211174:2;3135/chr11:4166:chr11:119211303-119211174:1;3136/chr8:
1696:chr8:53853072-53852897:2;3137/chr20:925:chr20:32255805-32255632:2;3138/chr20:925:chr20:3225580
5-32255632:1;3139/chr1:5019:chr1:119527248-119527074:2;3140/chr1:5019:chr1:119527248-119527074:1;31
41/chr3:3168:chr3:184301987-184301858:2;3142/chr3:3168:chr3:184301987-184301858:1;3143/cg21187769:c
hr4:93226640-93226467:2;3144/chr4:616:chr4:3734376-3734207:1;3145/chr11:4166:chr11:119211415-119211
262:2;3146/chr11:4166:chr11:119211415-119211262:1;3147/cg21187769:chr4:93226640-93226467:1;3148/chr
7:4153:chr7:150812832-150812659:2;3149/chr7:4153:chr7:150812832-150812659:1;3150/chr2:5245:chr2:2370
81933-237081785:2;3151/chr2:5245:chr2:237081933-237081785:1;3152/chr17:1256:chr17:17713674-1771350
4:2;3153/chr17:1256:chr17:17713674-17713504:1;3154/chr21:333:chr21:38081461-38081332:2;3155/chr21:33
3:chr21:38081461-38081332:1;3156/chr8:1696:chr8:53853072-53852897:1;3157/chrX:666:chrX:39865130-398
65005:2;3158/chrX:666:chrX:39865130-39865005:1;3159/chr4:1277:chr4:8873370-8873236:2;3160/chr4:1277:
chr4:8873370-8873236:1;3161/chr8:1695:chr8:53852447-53852282:2;3162/chr8:1695:chr8:53852447-5385228
2:1;3163/chr11:2279:chr11:63530454-63530313:2;3164/chr11:2279:chr11:63530454-63530313:1;3165/chr16:4
109:chr16:88496998-88496825:2;3166/chr16:4109:chr16:88496998-88496825:1;3167/chr2:4079:chr2:1715703
06-171570131:1;3168/chr2:4451:chr2:200328937-200328764:1;3169/chr6:2396:chr6:117869420-117869252:2;
3170/chr6:2396:chr6:117869420-117869252:1;3171/chr12:2942:chr12:128752143-128751971:2;3172/chr12:29
42:chr12:128752143-128751971:1;3173/chr9:629:chr9:79635846-79635721:1;3174/chr22:2774:chr22:5104289
1-51042718:2;3175/chr22:2774:chr22:51042891-51042718:1;3176/chr19:3827:chr19:46387963-46387790:2;31
77/chr19:3827:chr19:46387963-46387790:1;3178/chr22:2727:chr22:50738414-50738269:2;3179/chr22:2727:ch
r22:50738414-50738269:1;3180/chr11:2492:chr11:65314060-65313924:2;3181/chr11:2492:chr11:65314060-65
313924:1;3182/chr15:1843:chr15:84323323-84323148:1;3183/chr1:3957:chr1:47905472-47905343:2;3184/chr1:
3957:chr1:47905472-47905343:1;3185/chr10:2795:chr10:103539289-103539116:2;3186/chr10:2795:chr10:103
539289-103539116:1;3187/chr10:3746:chr10:129537390-129537215:2;3188/chr10:3746:chr10:129537390-129
537215:1;3189/chr13:45:chr13:20176421-20176252:2;3190/chr13:45:chr13:20176421-20176252:1;3191/chr4:1
479:chr4:20255532-20255360:2;3192/chr4:1479:chr4:20255532-20255360:1;3193/chr10:2605:chr10:10128211
6-101281941:2;3194/chr10:2605:chr10:101282116-101281941:1;3195/chr12:1427:chr12:57619134-57618961:2;
3196/chr12:1427:chr12:57619134-57618961:1;3197/chr7:874:chr7:3341572-3341409:2;3198/chr7:874:chr7:33
41572-3341409:1;3199/chr17:2925:chr17:62775270-62775100:2;3200/chr17:2925:chr17:62775270-62775100:1;
3201/chr3:2383:chr3:128211497-128211324:2;3202/chr3:2383:chr3:128211497-128211324:1;3203/chr13:1076:
chr13:100641905-100641731:1;3204/chr16:2474:chr16:54970525-54970351:2;3205/chr16:2474:chr16:549705
25-54970351:1;3206/chr1:4566:chr1:86622710-86622548:2;3207/chr1:4566:chr1:86622710-86622548:1;3208/c
hr1:3867:chr1:45083143-45082977:2;3209/chr1:3867:chr1:45083143-45082977:1;3210/chr22:1823:chr22:4636
8564-46368397:2;3211/chr22:1823:chr22:46368564-46368397:1;3212/chr2:2322:chr2:85361686-85361514:2;3
213/chr2:2322:chr2:85361686-85361514:1;3214/chr7:4146:chr7:150778859-150778685:2;3215/chr7:4146:chr7:
150778859-150778685:1;3216/chr14:1435:chr14:93154502-93154356:2;3217/chr14:1435:chr14:93154502-931
54356:1;3218/chr12:1447:chr12:58013562-58013407:2;3219/chr12:1447:chr12:58013562-58013407:1;3220/chr
8:1752:chr8:56015131-56014961:2;3221/chr8:1752:chr8:56015131-56014961:1;3222/chr11:2460:chr11:648151
61-64814993:2;3223/chr11:2460:chr11:64815161-64814993:1;3224/chr14:125:chr14:24045639-24045464:2;32
25/chr14:125:chr14:24045639-24045464:1;3226/chr10:2140:chr10:81003487-81003359:2;3227/chr10:2140:chr
10:81003487-81003359:1;3228/chr5:2932:chr5:158737763-158737588:2;3229/chr5:2932:chr5:158737763-1587
37588:1;3230/chr6:2396:chr6:117869606-117869463:2;3231/chr6:2396:chr6:117869606-117869463:1;3232/chr
3:2518:chr3:133393546-133393397:2;3233/chr3:2518:chr3:133393546-133393397:1;3234/chr22:1842:chr22:46
460493-46460322:1;3235/chr2:5038:chr2:233285594-233285465:2;3236/chr2:5038:chr2:233285594-23328546
5:1;3237/chr5:1812:chr5:77147813-77147685:2;3238/chr5:1812:chr5:77147813-77147685:1;3239/cg20680720:
chr19:37407310-37407182:1;3240/chr8:1876:chr8:65282266-65282139:2;3241/chr8:1876:chr8:65282266-6528
2139:1;3242/chr20:534:chr20:21491711-21491581:2;3243/chr20:534:chr20:21491711-21491581:1;3244/chr3:2
770:chr3:147128194-147128051:2;3245/chr3:2770:chi3:147128194-147128051:1;3246/chr1:4300:chr1:590419
21-59041785:2;3247/chr1:4300:chr1:59041921-59041785:1;3248/chr14:987:chr14:73958627-73958453:1;3249/
chr14:164:chr14:24804272-24804118:2;3250/chr14:164:chr14:24804272-24804118:1;3251/chr7:4153:chr7:150
812911-150812783:2;3252/chr7:4153:chr7:150812911-150812783:1;3253/chr14:530:chr14:54421848-5442167
7:1;3254/chr3:2385:chr3:128212063-128211910:2;3255/chr3:2385:chr3:128212063-128211910:1;3256/chr7:37
63:chr7:139168180-139168029:2;3257/chr7:3763:chr7:139168180-139168029:1;3258/chr8:1006:chr8:2240908
8-22408941:2;3259/chr4:2250:chr4:111542474-111542319:2;3260/chr4:2250:chr4:111542474-111542319:1;32
61/chr8:1006:chr8:22409088-22408941:1;3262/chr19:369:chr19:1470170-1470019:2;3263/chr19:369:chr19:147
0170-1470019:1;3264/chr18:1140:chr18:67068177-67068018:2;3265/chr18:1140:chr18:67068177-67068018:1;
3266/chr1:3004:chr1:25257033-25256864:2;3267/chr1:3004:chr1:25257033-25256864:1;3268/chr5:705:chr5:27
51518-2751393:2;3269/chr5:705:chr5:2751518-2751393:1;3270/chr8:1971:chr8:72459870-72459707:2;3271/ch
r8:1971:chr8:72459870-72459707:1;3272/chr18:1360:chr18:74962504-74962343:2;3273/chr18:1360:chr18:749
62504-74962343:1;3274/chr1:3435:chr1:34642634-34642473:2;3275/chr1:3435:chr1:34642634-34642473:1;32
76/cg21900495:chr13:28498250-28498077:1;3277/chr11:3841:chr11:112834245-112834087:2;3278/chr11:384
1:chr11:112834245-112834087:1;3279/chr10:2140:chr10:81003767-81003594:2;3280/chr10:2140:chr10:81003
767-81003594:1;3281/chr4:2398:chr4:135122725-135122590:2;3282/chr4:2398:chr4:135122725-135122590:1;
3283/chr2:2164:chr2:73147930-73147765:2;3284/chr2:2164:chr2:73147930-73147765:1;3285/chr3:2471:chr3:1
29694024-129693888:2;3286/chr3:2471:chr3:129694024-129693888:1;3287/chr1:7109:chr1:234040716-23404
0556:2;3288/chr1:7109:chr1:234040716-234040556:1;3289/chr11:3664:chr11:94277863-94277697:2;3290/chr6:
3360:chr6:168502655-168502481:2;3291/chr6:3360:chr6:168502655-168502481:1;3292/chr18:1835:chr18:775
48266-77548141:2;3293/chr18:1835:chr18:77548266-77548141:1;3294/chr11:3664:chr11:94277863-94277697:
1;3295/chr10:2676:chr10:102507768-102507609:2;3296/chr10:2676:chr10:102507768-102507609:1;3297/chr2
0:765:chr20:26190013-26189871:2;3298/chr20:765:chr20:26190013-26189871:1;3299/chr22:826:chr22:334544
25-33454255:2;3300/chr22:826:chr22:33454425-33454255:1;3301/chr5:2942:chr5:159399240-159399100:2;33
02/chr5:2942:chr5:159399240-159399100:1;3303/chr6:2232:chr6:107956201-107956055:2;3304/chr6:2232:chr
6:107956201-107956055:1;3305/chr13:1108:chr13:102569333-102569165:2;3306/chr13:1108:chr13:10256933
3-102569165:1;3307/chr15:937:chr15:58358555-58358414:2;3308/chr15:937:chr15:58358555-58358414:1;330
9/chr18:566:chr18:31020775-31020616:2;3310/chr18:566:chr18:31020775-31020616:1;3311/chr9:3509:chr9:13
9741333-139741192:2;3312/chr9:3509:chr9:139741333-139741192:1;3313/chr7:3100:chr7:99769560-9976939
2:2;3314/chr7:3100:chr7:99769560-99769392:1;3315/chr16:756:chr16:3016875-3016750:2;3316/cg19991022:c
hr20:53092034-53091870:2;3317/chr5:839:chr5:3602730-3602602:2;3318/chr5:839:chr5:3602730-3602602:1;3
319/chr4:2712:chr4:174428018-174427891:2;3320/chr4:2712:chr4:174428018-174427891:1;3321/chr16:756:ch
r16:3016875-3016750:1;3322/cg19991022:chr20:53092034-53091870:1;3323/chr6:1147:chr6:33245572-33245
407:2;3324/chr6:1147:chr6:33245572-33245407:1;3325/chr13:341:chr13:28498359-28498188:2;3326/chr13:34
1:chr13:28498359-28498188:1;3327/chr17:1855:chr17:36667075-36666950:2;3328/chr17:1855:chr17:3666707
5-36666950:1;3329/chr14:411:chr14:38724651-38724486:2;3330/chr14:411:chr14:38724651-38724486:1;3331/
chr7:2146:chr7:49815018-49814847:2;3332/chr7:2146:chr7:49815018-49814847:1;3333/chr8:1971:chr8:72460
256-72460113:1;3334/chr7:562:chr7:1626476-1626339:1;3335/chr8:565:chr8:8559923-8559770:2;3336/chr2:41
91:chr2:175205500-175205375:2;3337/chr2:4191:chr2:175205500-175205375:1;3338/cg06825142:chr11:6372
31-637068:2;3339/cg00556112:chr11:637231-637068:2;3340/cg12928379:chr11:637231-637068:2;3341/cg068
25142:chr11:637231-637068:1;3342/cg00556112:chr11:637231-637068:1;3343/cg12928379:chr11:637231-637
068:1;3344/chr8:565:chr8:8559923-8559770:1;3345/chr2:3847:chr2:145282046-145281888:2;3346/chr2:3847:c
hr2:145282046-145281888:1;3347/chr6:1289:chr6:35109233-35109059:2;3348/chr6:1289:chr6:35109233-3510
9059:1;3349/chr2:1263:chr2:26407764-26407601:2;3350/chr2:1263:chr2:26407764-26407601:1;3351/chr11:55
0:chr11:1995573-1995413:2;3352/chr11:550:chr11:1995573-1995413:1;3353/chr8:3689:chr8:145052201-14505
2039:2;3354/chr8:3689:chr8:145052201-145052039:1;3355/chr20:1878:chr20:55965485-55965360:2;3356/chr2
0:1878:chr20:55965485-55965360:1;3357/chr17:2925:chr17:62775327-62775153:2;3358/chr22:2703:chr22:506
23625-50623499:2;3359/chr22:2703:chr22:50623625-50623499:1;3360/chr8:2963:chr8:142428248-142428073:
2;3361/chr8:2963:chr8:142428248-142428073:1;3362/cg11855526:chr11:30607171-30607011:2;3363/cg11855
526:chr11:30607171-30607011:1;3364/chr6:99:chr6:1384495-1384359:2;3365/chr6:99:chr6:1384495-1384359:
1;3366/chr22:1842:chr22:46460571-46460436:1;3367/cg16629469:chr6:108440719-108440544:2;3368/cg1662
9469:chr6:108440719-108440544:1;3369/chr1:4313:chr1:60280484-60280341:2;3370/chr1:4313:chr1:6028048
4-60280341:1;3371/chr7:3399:chr7:112430452-112430282:2;3372/chr7:3399:chr7:112430452-112430282:1;33
73/chr19:3999:chr19:48947026-48946885:2;3374/chr19:3999:chr19:48947026-48946885:1;3375/chr17:176:chr
17:1174373-1174198:2;3376/chr17:176:chr17:1174373-1174198:1;3377/chr15:1086:chr15:65116415-65116278:
2;3378/chr15:1086:chr15:65116415-65116278:1;3379/chr4:123:chr4:997096-996932:2;3380/chr4:123:chr4:997
096-996932:1;3381/chr17:2925:chr17:62775327-62775153:1;3382/chr14:354:chr14:37128041-37127887:2;338
3/chr14:354:chr14:37128041-37127887:1;3384/chr10:906:chr10:23481148-23481004:2;3385/chr10:906:chr10:2
3481148-23481004:1;3386/chr19:4944:chr19:58908071-58907909:2;3387/chr19:4944:chr19:58908071-589079
09:1;3388/chr1:135:chr1:1118535-1118364:2;3389/chr1:135:chr1:1118535-1118364:1;3390/chr4:1288:chr4:889
5173-8895007:2;3391/chr4:1288:chr4:8895173-8895007:1;3392/chr11:1916:chr11:47416578-47416404:2;3393/
chr11:1916:chr11:47416578-47416404:1;3394/chr16:549:chr16:1583988-1583819:2;3395/chr16:549:chr16:158
3988-1583819:1;3396/chr13:100:chr13:21296040-21295897:2;3397/chr13:100:chr13:21296040-21295897:1;33
98/chr1:3805:chr1:44031917-44031742:2;3399/chr1:3805:chr1:44031917-44031742:1;3400/chr22:162:chr22:19
711747-19711621:2;3401/chr22:162:chr22:19711747-19711621:1;3402/chr3:431:chr3:13590756-13590630:2;3
403/chr3:431:chr3:13590756-13590630:1;3404/chr5:2252:chr5:132149962-132149834:2;3405/chr5:2252:chr5:1
32149962-132149834:1;3406/chr16:1604:chr16:23847181-23847047:2;3407/chr16:1604:chr16:23847181-2384
7047:1;3408/chr16:1299:chr16:12996156-12996019:2;3409/chr16:1299:chr16:12996156-12996019:1;3410/chr1
0:2605:chr10:101282156-101281993:2;3411/chr10:2605:chr10:101282156-101281993:1;3412/chr1:5568:chr1:1
61009041-161008866:2;3413/chr1:5568:chr1:161009041-161008866:1;3414/chr4:773:chr4:5894393-5894224:2;
3415/chr4:773:chr4:5894393-5894224:1;3416/chr10:1053:chr10:30025887-30025712:2;3417/chr10:1053:chr10:
30025887-30025712:1;3418/chr9:1705:chr9:126774872-126774706:2;3419/chr9:1705:chr9:126774872-126774
706:1;3420/chr15:2482:chr15:101267771-101267608:2;3421/chr15:2482:chr15:101267771-101267608:1;3422/
chr10:3180:chr10:118899656-118899517:2;3423/chr10:3180:chr10:118899656-118899517:1;3424/chr11:4765:
chr11:131780894-131780749:2;3425/chr11:4765:chr11:131780894-131780749:1;3426/chr14:688:chr14:617472
92-61747130:2;3427/chr14:688:chr14:61747292-61747130:1;3428/chr15:2501:chr15:101513499-101513333:2;
3429/chr15:2501:chr15:101513499-101513333:1;3430/chr16:2161:chr16:49316543-49316374:2;3431/chr16:21
61:chr16:49316543-49316374:1;3432/chr11:2620:chr11:66885446-66885280:2;3433/chr18:610:chr18:3483402
4-34833860:2;3434/chr18:610:chr18:34834024-34833860:1;3435/chr6:245:chr6:4776266-4776119:2;3436/chr6:
245:chr6:4776266-4776119:1;3437/chr14:2206:chr14:103674237-103674071:2;3438/chr1:252:chr1:1565508-15
65333:2;3439/chr14:2206:chr14:103674237-103674071:1;3440/chr1:252:chr1:1565508-1565333:1;3441/chr2:4
252:chr2:176987721-176987565:2;3442/chr2:4252:chr2:176987721-176987565:1;3443/chr14:2727:chr14:1057
14621-105714491:2;3444/chr14:2727:chr14:105714621-105714491:1;3445/chr6:256:chr6:5086691-5086526:2;
3446/chr6:256:chr6:5086691-5086526:1;3447/chr2:1903:chr2:63275646-63275475:2;3448/chr2:1903:chr2:6327
5646-63275475:1;3449/chr13:708:chr13:53420330-53420172:2;3450/chr13:708:chr13:53420330-53420172:1;3
451/chr2:4772:chr2:220313729-220313589:2;3452/chr2:4772:chr2:220313729-220313589:1;3453/chr3:2897:ch
r3:157821338-157821173:2;3454/chr3:2897:chr3:157821338-157821173:1;3455/chr18:75:chr18:4454816-4454
666:2;3456/chr18:75:chr18:4454816-4454666:1;3457/chr16:1666:chr16:25704281-25704106:2;3458/chr16:166
6:chr16:25704281-25704106:1;3459/chr22:1921:chr22:46932700-46932571:2;3460/chr22:1921:chr22:4693270
0-46932571:1;3461/chr9:239:chr9:23821832-23821668:2;3462/chr9:239:chr9:23821832-23821668:1;3463/chr1
0:503:chr10:8076342-8076173:2;3464/chr10:503:chr10:8076342-8076173:1;3465/chr3:220:chr3:10857834-108
57680:1;3466/chr4:1701:chr4:42153533-42153388:2;3467/chr4:1701:chr4:42153533-42153388:1;3468/chr11:2
620:chr11:66885446-66885280:1;3469/chr13:1184:chr13:109793186-109793046:2;3470/chr13:1184:chr13:109
793186-109793046:1;3471/chr19:4874:chr19:58238954-58238790:2;3472/chr19:4874:chr19:58238954-582387
90:1;3473/chr4:2130:chr4:91049064-91048910:2;3474/chr4:2130:chr4:91049064-91048910:1;3475/chr3:392:ch
r3:13324334-13324183:2;3476/chr2:3718:chr2:131797905-131797741:2;3477/chr2:3718:chr2:131797905-1317
97741:1;3478/chr3:392:chr3:13324334-13324183:1;3479/chr8:3702:chr8:145105618-145105455:2;3480/chr8:3
702:chr8:145105618-145105455:1;3481/chr19:3840:chr19:46518488-46518359:2;3482/chr19:3840:chr19:4651
8488-46518359:1;3483/chr2:3999:chr2:162283660-162283508:2;3484/chr2:3999:chr2:162283660-162283508:1;
3485/chr15:2554:chr15:102029807-102029665:2;3486/chr15:2554:chr15:102029807-102029665:1;3487/chr8:3
9:chr8:687766-687635:2;3488/chr8:39:chr8:687766-687635:1;3489/chr4:391:chr4:2042667-2042498:2;3490/chr
4:391:chr4:2042667-2042498:1;3491/chr17:273:chr17:1960077-1959921:2;3492/chr17:273:chr17:1960077-195
9921:1;3493/chr11:99:chr11:637042-636886:2;3494/chr11:99:chr11:637042-636886:1;3495/chr5:838:chr5:3600
908-3600748:2;3496/chr5:838:chr5:3600908-3600748:1;3497/chr21:100:chr21:27945480-27945338:2;3498/chr
21:100:chr21:27945480-27945338:1;3499/chr8:653:chr8:10589111-10588937:1;3500/chr4:5:chr4:107334-1071
82:2;3501/cg03048083:chr17:43339537-43339389:2;3502/cg03048083:chr17:43339537-43339389:1;3503/chr1
6:4109:chr16:88497027-88496899:2;3504/chr16:4109:chr16:88497027-88496899:1;3505/chr4:5:chr4:107334-1
07182:1;3506/chr10:2027:chr10:77159421-77159248:2;3507/chr10:2027:chr10:77159421-77159248:1;3508/chr
7:4539:chr7:155580135-155579998:2;3509/chr7:4539:chr7:155580135-155579998:1;3510/chr10:848:chr10:217
99199-21799041:1;3511/chr19:158:chr19:863092-862967:2;3512/chr19:158:chr19:863092-862967:1;3513/chr1
7:551:chr17:5001082-5000914:2;3514/chr17:551:chr17:5001082-5000914:1;3515/chrX:810:chrX:48685568-486
85417:2;3516/chrX:810:chrX:48685568-48685417:1;3517/chr12:157:chr12:2800967-2800835:2;3518/chr12:157:
chr12:2800967-2800835:1;3519/chr5:1725:chr5:72732498-72732354:2;3520/chr5:1725:chr5:72732498-727323
54:1;3521/chr19:2488:chr19:29285026-29284874:1;3522/chr17:2823:chr17:59529673-59529528:2;3523/chr17:
2823:chr17:59529673-59529528:1;3524/chr4:62:chr4:683039-682887:2;3525/chr4:62:chr4:683039-682887:1;35
26/chr2:4251:chr2:176987386-176987216:2;3527/chr2:4251:chr2:176987386-176987216:1;3528/chr19:4653:ch
r19:55598286-55598119:2;3529/chr19:4653:chr19:55598286-55598119:1;3530/chr17:2810:chr17:59482706-59
482541:2;3531/chr17:2810:chr17:59482706-59482541:1;3532/chr13:498:chr13:36045231-36045095:1;3533/chr
6:1746:chr6:50818289-50818134:1;3534/chr1:6194:chr1:203045133-203044961:2;3535/chr10:2698:chr10:1025
86829-102586659:2;3536/chr10:2698:chr10:102586829-102586659:1;3537/chr12:2984:chr12:129337950-1293
37818:2;3538/chr12:2984:chr12:129337950-129337818:1;3539/chr1:6194:chr1:203045133-203044961:1;3540/
chr2:2215:chr2:74725245-74725077:2;3541/chr2:2215:chr2:74725245-74725077:1;3542/chr2:4132:chr2:17296
5651-172965477:2;3543/chr2:4132:chr2:172965651-172965477:1;3544/chr1:2566:chr1:18434531-18434373:2;
3545/chr1:2566:chr1:18434531-18434373:1;3546/chr17:2750:chr17:56564942-56564787:2;3547/chr17:2750:ch
r17:56564942-56564787:1;3548/chr1:5025:chr1:119532199-119532043:2;3549/chr1:5025:chr1:119532199-119
532043:1;3550/chr3:2753:chr3:147111756-147111581:2;3551/chr3:2753:chr3:147111756-147111581:1;3552/c
hr3:333:chr3:13114910-13114767:2;3553/chr3:333:chr3:13114910-13114767:1;3554/chr9:1705:chr9:12677513
1-126774956:2;3555/chr19:4369:chr19:52552234-52552059:1;3556/chr10:1637:chr10:60937050-60936875:2;3
557/chr10:1637:chr10:60937050-60936875:1;3558/chr6:279:chr6:6003997-6003844:2;3559/chr6:279:chr6:6003
997-6003844:1;3560/chr9:1705:chr9:126775131-126774956:1;3561/chr16:241:chr16:1041040-1040915:2;3562/
chr16:241:chr16:1041040-1040915:1;3563/cg22871668:chr6:133562602-133562455:2;3564/cg22871668:chr6:
133562602-133562455:1;3565/chr14:1863:chr14:100626301-100626150:2;3566/chr14:1863:chr14:100626301-
100626150:1;3567/chr16:2379:chr16:51168673-51168541:2;3568/chr16:2379:chr16:51168673-51168541:1;356
9/chr12:1460:chr12:58130245-58130113:2;3570/chr12:1797:chr12:99289255-99289086:2;3571/chr12:1797:chr
12:99289255-99289086:1;3572/chr15:2091:chr15:91427854-91427727:2;3573/chr15:2091:chr15:91427854-914
27727:1;3574/chr14:354:chr14:37128234-37128109:2;3575/chr14:354:chr14:37128234-37128109:1;3576/chr2:
4191:chr2:175205614-175205487:2;3577/chr2:4191:chr2:175205614-175205487:1;3578/chr22:2792:chr22:511
36490-51136319:2;3579/chr22:2792:chr22:51136490-51136319:1;3580/chr18:763:chr18:44336707-44336534:2;
3581/chr18:763:chr18:44336707-44336534:1;3582/chr16:4179:chr16:88600853-88600716:2;3583/chr16:4179:
chr16:88600853-88600716:1;3584/chr8:3527:chr8:144358223-144358050:2;3585/chr8:3527:chr8:144358223-1
44358050:1;3586/chr18:1054:chr18:57364205-57364078:2;3587/chr18:1054:chr18:57364205-57364078:1;3588/
chr10:3506:chr10:125651373-125651228:2;3589/chr10:3506:chr10:125651373-125651228:1;3590/chr11:3710:
chr11:100998783-100998652:2;3591/chr11:3710:chr11:100998783-100998652:1;3592/chr1:7249:chr1:2402563
50-240256176:2;3593/chr1:7249:chr1:240256350-240256176:1;3594/chr11:3716:chr11:101454600-101454475:
2;3595/chr11:3716:chr11:101454600-101454475:1;3596/chr19:3999:chr19:48947449-48947313:2;3597/chr19:3
999:chr19:48947449-48947313:1;3598/chr8:96:chr8:877194-877051:2;3599/chr22:1852:chr22:46475997-46475
855:1;3600/chr8:96:chr8:877194-877051:1;3601/chr20:136:chr20:2674423-2674255:2;3602/chr20:136:chr20:26
74423-2674255:1;3603/chr7:735:chr7:2558637-2558467:2;3604/chr3:138:chr3:8810159-8809991:2;3605/chr13:
1468:chr13:112727596-112727432:2;3606/chr13:1468:chr13:112727596-112727432:1;3607/chr3:138:chr3:881
0159-8809991:1;3608/chr7:735:chr7:2558637-2558467:1;3609/chr12:1112:chr12:51718272-51718144:2;3610/c
hr12:1112:chr12:51718272-51718144:1;3611/chr1:5019:chr1:119527190-119527022:2;3612/chr9:1941:chr9:13
0461688-130461516:2;3613/chr9:1941:chr9:130461688-130461516:1;3614/chr5:3644:chr5:178004303-178004
161:2;3615/chr5:3644:chr5:178004303-178004161:1;3616/chrX:678:chrX:39949790-39949617:2;3617/chrX:678:
chrX:39949790-39949617:1;3618/chr14:411:chr14:38724949-38724806:2;3619/chr14:411:chr14:38724949-38
724806:1;3620/chr8:1921:chr8:67873735-67873571:2;3621/chr8:1921:chr8:67873735-67873571:1;3622/chr17:
1855:chr17:36666834-36666676:2;3623/chr17:1855:chr17:36666834-36666676:1;3624/chr8:1018:chr8:224469
18-22446781:1;3625/chr11:5:chr11:280409-280283:2;3626/chr11:5:chr11:280409-280283:1;3627/chr19:368:chr
19:1469851-1469717:2;3628/chr19:368:chr19:1469851-1469717:1;3629/chr7:1788:chr7:35293721-35293575:2;
3630/chr7:1788:chr7:35293721-35293575:1;3631/chr21:666:chr21:44037406-44037274:2;3632/chr21:666:chr2
1:44037406-44037274:1;3633/chr13:289:chr13:26625971-26625841:2;3634/chr13:289:chr13:26625971-266258
41:1;3635/chr9:1083:chr9:96722812-96722641:2;3636/chr9:1083:chr9:96722812-96722641:1;3637/chr7:1491:c
hr7:27153450-27153313:2;3638/chr7:1491:chr7:27153450-27153313:1;3639/chr15:2490:chr15:101390108-101
389933:2;3640/chr15:2490:chr15:101390108-101389933:1;3641/chr2:599:chr2:5833323-5833186:2;3642/chr2:
599:chr2:5833323-5833186:1;3643/chr12:1140:chr12:52301463-52301296:2;3644/chr12:1140:chr12:52301463-
52301296:1;3645/chr19:567:chr19:2291166-2291020:2;3646/chr19:567:chr19:2291166-2291020:1;3647/chr10:
2874:chr10:105344711-105344540:2;3648/chr10:2874:chr10:105344711-105344540:1;3649/chr1:3400:chr1:33
772454-33772282:2;3650/chr1:3400:chr1:33772454-33772282:1;3651/chr19:4653:chr19:55598159-55597989:2;
3652/chr5:1434:chr5:42952505-42952380:2;3653/chr5:1434:chr5:42952505-42952380:1;3654/chr19:4653:chr1
9:55598159-55597989:1;3655/chr10:2424:chr10:94822806-94822638:2;3656/chr10:2424:chr10:94822806-9482
2638:1;3657/chr18:768:chr18:44526816-44526641:1;3658/chr8:1971:chr8:72459926-72459753:2;3659/chr8:19
71:chr8:72459926-72459753:1;3660/cg03853987:chr2:101034388-101034257:2;3661/cg03853987:chr2:10103
4388-101034257:1;3662/chr20:2212:chr20:59828927-59828759:2;3663/chr20:2212:chr20:59828927-59828759:
1;3664/chr11:1916:chr11:47416649-47416519:2;3665/chr11:1916:chr11:47416649-47416519:1;3666/chr9:1956:
chr9:130516636-130516462:2;3667/chr9:1956:chr9:130516636-130516462:1;3668/chr17:2827:chr17:5953215
9-59531988:1;3669/chr7:4153:chr7:150812613-150812439:2;3670/chr6:2263:chr6:108489980-108489805:2;36
71/chr6:2263:chr6:108489980-108489805:1;3672/chr12:1319:chr12:54428065-54427925:2;3673/chr13:368:chr
13:28674422-28674269:2;3674/chr13:368:chr13:28674422-28674269:1;3675/chr6:384:chr6:10385040-1038491
4:2;3676/chr6:384:chr6:10385040-10384914:1;3677/chr17:309:chr17:2627811-2627636:2;3678/chr17:309:chr1
7:2627811-2627636:1;3679/chr13:289:chr13:26626263-26626088:2;3680/cg25664438:chr8:97505677-9750550
7:1;3681/chr4:15:chr4:330804-330668:2;3682/chr17:3473:chr17:73584368-73584202:2;3683/chr6:2396:chr6:11
7869487-117869358:2;3684/chr2:4191:chr2:175205215-175205047:1;3685/chr4:15:chr4:330804-330668:1;368
6/chr19:3503:chr19:41119463-41119326:2;3687/chr19:3503:chr19:41119463-41119326:1;3688/chr5:1949:chr5:
92939934-92939766:2;3689/chr5:1949:chr5:92939934-92939766:1;3690/chr1:5370:chr1:156046859-15604668
8:2;3691/chr7:4303:chr7:152623001-152622834:2;3692/chr22:2727:chr22:50738316-50738160:2;3693/chr14:2
041:chr14:101925249-101925120:2;3694/chr8:766:chr8:12809510-12809352:2;3695/chr8:766:chr8:12809510-1
2809352:1;3696/chr5:2729:chr5:149546940-149546775:2;3697/chr19:1696:chr19:13210644-13210519:2;3698/
chr19:1696:chr19:13210644-13210519:1;3699/chr17:1814:chr17:35300059-35299884:2;3700/chr12:51:chr12:6
79717-679591:2;3701/chr12:51:chr12:679717-679591:1;3702/chr17:1814:chr17:35300059-35299884:1;3703/ch
r4:2130:chr4:91049124-91048951:2;3704/chr4:2130:chr4:91049124-91048951:1;3705/chr10:808:chr10:184293
68-18429242:2;3706/chr10:808:chr10:18429368-18429242:1;3707/chr5:1172:chr5:15500644-15500504:2;3708/
chr5:1172:chr5:15500644-15500504:1;3709/chr4:2716:chr4:174430884-174430709:2;3710/chr4:2716:chr4:174
430884-174430709:1;3711/chr22:2727:chr22:50738316-50738160:1;3712/chr14:2041:chr14:101925249-10192
5120:1;3713/chr5:2729:chr5:149546940-149546775:1;3714/chr14:2140:chr14:103011303-103011133:2;3715/c
hr14:2140:chr14:103011303-103011133:1;3716/chr8:1744:chr8:55383071-55382932:2;3717/chr8:1744:chr8:55
383071-55382932:1;3718/chr4:1403:chr4:13544084-13543909:2;3719/chr4:1403:chr4:13544084-13543909:1;3
720/chr8:1403:chr8:38757800-38757625:2;3721/chr8:1403:chr8:38757800-38757625:1;3722/chr13:1702:chr13:
113764513-113764384:2;3723/chr13:1702:chr13:113764513-113764384:1;3724/chr7:4153:chr7:150812613-15
0812439:1;3725/chr5:514:chr5:1887279-1887153:2;3726/chr5:514:chr5:1887279-1887153:1;3727/chr12:1319:c
hr12:54428065-54427925:1;3728/chr8:1752:chr8:56014904-56014779:2;3729/chr8:1752:chr8:56014904-56014
779:1;3730/chr4:26:chr4:570064-569917:2;3731/chr4:26:chr4:570064-569917:1;3732/chr19:3157:chr19:370962
09-37096036:2;3733/chr19:3157:chr19:37096209-37096036:1;3734/chr8:3703:chr8:145106100-145105933:2;3
735/chr17:3473:chr17:73584368-73584202:1;3736/chr8:3703:chr8:145106100-145105933:1;3737/chr19:4796:c
hr19:56988883-56988716:2;3738/chr19:4796:chr19:56988883-56988716:1;3739/chr14:626:chr14:59931801-59
931629:2;3740/chr14:626:chr14:59931801-59931629:1;3741/chr1:5370:chr1:156046859-156046688:1;3742/chr
7:4303:chr7:152623001-152622834:1;3743/chr12:1282:chr12:54345893-54345719:2;3744/chr12:1282:chr12:54
345893-54345719:1;3745/chr19:918:chr19:4329039-4328904:2;3746/chr19:918:chr19:4329039-4328904:1;374
7/chr12:1614:chr12:72666920-72666747:2;3748/chr12:1614:chr12:72666920-72666747:1;3749/cg12473406:ch
r18:18823061-18822887:1;3750/chr1:3774:chr1:43534042-43533873:2;3751/chr1:3774:chr1:43534042-435338
73:1;3752/chr19:3873:chr19:46996967-46996794:2;3753/chr19:3873:chr19:46996967-46996794:1;3754/chr5:2
048:chr5:112539175-112539000:2;3755/chr22:1852:chr22:46476633-46476508:1;3756/chr16:1850:chr16:2988
8157-29888016:2;3757/chr8:33:chr8:638383-638208:2;3758/chr8:33:chr8:638383-638208:1;3759/chr20:2210:c
hr20:59828390-59828250:2;3760/chr20:2210:chr20:59828390-59828250:1;3761/chr1:5027:chr1:119533119-11
9532994:2;3762/chr1:5027:chr1:119533119-119532994:1;3763/chr17:1256:chr17:17713588-17713415:2;3764/
chr17:1256:chr17:17713588-17713415:1;3765/cg07352438:chr18:5543692-5543517:2;3766/cg07352438:chr18:
5543692-5543517:1;3767/chr16:1850:chr16:29888157-29888016:1;3768/chr10:3131:chr10:118031062-118030
891:2;3769/chr10:3131:chr10:118031062-118030891:1;3770/chr2:3710:chr2:131721958-131721806:2;3771/chr
2:3710:chr2:131721958-131721806:1;3772/chr8:728:chr8:11537877-11537751:2;3773/chr8:728:chr8:11537877-
11537751:1;3774/chr15:1198:chr15:68119312-68119144:2;3775/chr15:1198:chr15:68119312-68119144:1;377
6/cg10292139:chr8:97507685-97507560:1;3777/chr16:3825:chr16:86544989-86544823:2;3778/chr16:3825:chr
16:86544989-86544823:1;3779/chrX:1560:chrX:133684896-133684722:2;3780/chrX:1560:chrX:133684896-133
684722:1;3781/chr6:2142:chr6:100062041-100061876:2;3782/chr6:2142:chr6:100062041-100061876:1;3783/c
hr22:34:chr22:17566830-17566699:2;3784/chr22:34:chr22:17566830-17566699:1;3785/chr7:1422:chr7:258927
04-25892570:2;3786/chr11:2492:chr11:65314498-65314324:2;3787/chr11:2492:chr11:65314498-65314324:1;3
788/chr4:2973:chr4:188916732-188916557:2;3789/chr4:2973:chr4:188916732-188916557:1;3790/chr17:551:ch
r17:5001005-5000870:2;3791/chr17:551:chr17:5001005-5000870:1;3792/chr2:257:chr2:1747266-1747141:2;37
93/chr2:257:chr2:1747266-1747141:1;3794/chr4:202:chr4:1398927-1398756:2;3795/chr4:202:chr4:1398927-13
98756:1;3796/chr3:761:chr3:32859120-32858950:2;3797/chr3:761:chr3:32859120-32858950:1;3798/chr6:2842:
chr6:157558007-157557843:2;3799/chr6:2842:chr6:157558007-157557843:1;3800/chr6:384:chr6:10385214-10
385039:2;3801/chr9:3185:chr9:138675044-138674918:2;3802/chr9:3185:chr9:138675044-138674918:1;3803/c
hr14:323:chr14:36987134-36986989:2;3804/chr14:323:chr14:36987134-36986989:1;3805/chr7:4153:chr7:1508
12120-150811950:2;3806/chr7:4153:chr7:150812120-150811950:1;3807/chr15:1830:chr15:83952243-8395206
8:2;3808/chr15:1830:chr15:83952243-83952068:1;3809/chr8:652:chr8:10588404-10588268:2;3810/chr8:652:ch
r8:10588404-10588268:1;3811/chr17:534:chr17:4802497-4802360:2;3812/chr17:534:chr17:4802497-4802360:
1;3813/chr8:2179:chr8:97157378-97157203:2;3814/chr8:2179:chr8:97157378-97157203:1;3815/chr5:1826:chr5:
77806294-77806157:2;3816/chr5:1826:chr5:77806294-77806157:1;3817/chr2:3000:chr2:110372732-11037259
4:2;3818/chr2:3000:chr2:110372732-110372594:1;3819/chr7:3502:chr7:127672478-127672348:2;3820/chr7:35
02:chr7:127672478-127672348:1;3821/chr14:312:chr14:36973369-36973201:2;3822/chr14:312:chr14:3697336
9-36973201:1;3823/chr8:652:chr8:10588685-10588558:2;3824/chr8:652:chr8:10588685-10588558:1;3825/chr1
0:1133:chr10:35928914-35928762:2;3826/chr10:1133:chr10:35928914-35928762:1;3827/chr13:1060:chr13:100
608303-100608130:2;3828/chr13:1060:chr13:100608303-100608130:1;3829/chr16:825:chr16:3550241-355006
7:2;3830/chr13:561:chr13:43148450-43148276:2;3831/chr13:561:chr13:43148450-43148276:1;3832/chr16:679:
chr16:2286989-2286841:2;3833/chr16:679:chr16:2286989-2286841:1;3834/chr7:1932:chr7:42267824-4226765
2:2;3835/chr7:1932:chr7:42267824-42267652:1;3836/chr8:3527:chr8:144358287-144358122:2;3837/chr8:3527:
chr8:144358287-144358122:1;3838/chr3:161:chr3:9595671-9595499:2;3839/chr3:161:chr3:9595671-9595499:1;
3840/chr10:524:chr10:8098061-8097888:2;3841/chr10:524:chr10:8098061-8097888:1;3842/cg19283506:chr2:
101034382-101034257:2;3843/cg19283506:chr2:101034382-101034257:1;3844/chr12:1797:chr12:99289173-9
9289039:2;3845/chr2:38:chr2:468468-468303:1;3846/chr12:1797:chr12:99289173-99289039:1;3847/chr1:4861:
chr1:111217041-111216875:2;3848/chr1:4861:chr1:111217041-111216875:1;3849/chr2:3916:chr2:154335258-
154335083:2;3850/chr2:3916:chr2:154335258-154335083:1;3851/chr20:2056:chr20:57766664-57766525:2;385
2/chr20:2056:chr20:57766664-57766525:1;3853/chr10:2401:chr10:94449707-94449582:2;3854/chr10:2401:chr
10:94449707-94449582:1;3855/chr1:3209:chr1:29447932-29447791:2;3856/chr1:3209:chr1:29447932-2944779
1:1;3857/chr2:4772:chr2:220313037-220312912:2;3858/chr2:4772:chr2:220313037-220312912:1;3859/chr22:2
526:chr22:49969696-49969522:1;3860/chr4:1288:chr4:8895032-8894859:2;3861/chr12:1757:chr12:95942240-9
5942108:2;3862/chr12:1757:chr12:95942240-95942108:1;3863/chr12:1084:chr12:50297881-50297756:2;3864/
chr12:1084:chr12:50297881-50297756:1;3865/chr11:2488:chr11:65246497-65246336:2;3866/chr11:2488:chr11:
65246497-65246336:1;3867/chr7:2651:chr7:71801371-71801237:2;3868/chr7:2651:chr7:71801371-71801237:
1;3869/chr2:4572:chr2:209271691-209271523:2;3870/chr2:4572:chr2:209271691-209271523:1;3871/chr16:407
1:chr16:88443096-88442965:2;3872/chr16:4071:chr16:88443096-88442965:1;3873/chr22:2722:chr22:5070635
5-50706193:2;3874/chr22:2722:chr22:50706355-50706193:1;3875/chr21:158:chr21:32931512-32931343:2;387
6/chr21:158:chr21:32931512-32931343:1;3877/chr1:4364:chr1:63786045-63785873:2;3878/chr1:4364:chr1:637
86045-63785873:1;3879/chr5:1719:chr5:72678163-72677997:2;3880/chr5:1719:chr5:72678163-72677997:1;38
81/chr10:1053:chr10:30026071-30025937:2;3882/chr14:2099:chr14:102172127-102172000:2;3883/chr14:2099:
chr14:102172127-102172000:1;3884/chr8:13:chr8:494441-494270:2;3885/chr8:13:chr8:494441-494270:1;3886/
chr2:4183:chr2:175200535-175200387:2;3887/chr2:4183:chr2:175200535-175200387:1;3888/chr2:3031:chr2:1
11877174-111877048:2;3889/chr2:3031:chr2:111877174-111877048:1;3890/chr4:158:chr4:1165120-1164952:2;
3891/chr4:158:chr4:1165120-1164952:1;3892/chr12:1319:chr12:54427831-54427662:2;3893/chr12:1319:chr12:
54427831-54427662:1;3894/chr18:1613:chr18:76740551-76740411:2;3895/chr18:1613:chr18:76740551-76740
411:1;3896/chr14:881:chr14:70038453-70038328:2;3897/chr14:881:chr14:70038453-70038328:1;3898/chr9:19
56:chr9:130516899-130516766:2;3899/chr9:1956:chr9:130516899-130516766:1;3900/cg08848774:chr17:4304
7796-43047626:2;3901/cg08848774:chr17:43047796-43047626:1;3902/chr18:1155:chr18:70535128-70534960:
2;3903/chr18:1155:chr18:70535128-70534960:1;3904/chr17:1933:chr17:38334250-38334083:2;3905/chr17:193
3:chr17:38334250-38334083:1;3906/chr12:3052:chr12:130388709-130388536:2;3907/chr12:3052:chr12:13038
8709-130388536:1;3908/chr2:3049:chr2:112939312-112939180:2;3909/chr2:3049:chr2:112939312-112939180:
1;3910/chr17:534:chr17:4802768-4802599:2;3911/chr17:534:chr17:4802768-4802599:1;3912/chr10:1053:chr10:
30026071-30025937:1;3913/chr10:2424:chr10:94823007-94822879:2;3914/chr10:2424:chr10:94823007-94822
879:1;3915/chr1:4313:chr1:60280813-60280643:2;3916/chr1:4313:chr1:60280813-60280643:1;3917/chr10:195
8:chr10:73847967-73847792:2;3918/chr10:1958:chr10:73847967-73847792:1;3919/chr7:4303:chr7:152623305-
152623132:2;3920/chr7:4303:chr7:152623305-152623132:1;3921/chr2:4287:chr2:177043362-177043237:2;392
2/chr2:4287:chr2:177043362-177043237:1;3923/chr2:3447:chr2:127414109-127413935:2;3924/chr15:935:chr1
5:58357378-58357203:2;3925/chr12:1090:chr12:50355661-50355511:2;3926/chr12:1090:chr12:50355661-5035
5511:1;3927/chr5:1789:chr5:76373336-76373170:2;3928/chr5:1789:chr5:76373336-76373170:1;3929/chr10:90
5:chr10:23480939-23480766:2;3930/chr10:905:chr10:23480939-23480766:1;3931/chr8:1405:chr8:38832132-38
831961:2;3932/chr8:1405:chr8:38832132-38831961:1;3933/chr6:2190:chr6:105389450-105389282:2;3934/chr6:
2190:chr6:105389450-105389282:1;3935/chr15:634:chr15:41794989-41794814:2;3936/chr14:881:chr14:70038
575-70038408:2;3937/chr14:881:chr14:70038575-70038408:1;3938/chr13:1173:chr13:109148150-109147976:2;
3939/chr13:1173:chr13:109148150-109147976:1;3940/chr15:634:chr15:41794989-41794814:1;3941/chr15:207:
chr15:27112876-27112725:2;3942/chr15:207:chr15:27112876-27112725:1;3943/chr2:1926:chr2:63287140-632
86966:2;3944/chr7:4818:chr7:157478929-157478804:2;3945/chr2:1926:chr2:63287140-63286966:1;3946/chr7:
4818:chr7:157478929-157478804:1;3947/chr8:1405:chr8:38832285-38832115:2;3948/chr8:1405:chr8:3883228
5-38832115:1;3949/chr14:393:chr14:38067595-38067427:2;3950/chr14:393:chr14:38067595-38067427:1;3951/
chr17:4422:chr17:79315678-79315507:2;3952/chr17:4422:chr17:79315678-79315507:1;3953/chr8:1405:chr8:3
8831964-38831800:2;3954/chr8:1405:chr8:38831964-38831800:1;3955/chr17:1003:chr17:11144167-11144024:
2;3956/chr18:503:chr18:24130728-24130562:1;3957/chr10:4269:chr10:133110014-133109862:2;3958/chr10:42
69:chr10:133110014-133109862:1;3959/chr3:761:chr3:32859021-32858892:2;3960/chr17:658:chr17:6899728-6
899553:2;3961/chr17:658:chr17:6899728-6899553:1;3962/chr3:761:chr3:32859021-32858892:1;3963/chr8:354:
chr8:1950567-1950398:2;3964/chr8:354:chr8:1950567-1950398:1;3965/chr10:3406:chr10:123923312-1239231
45:2;3966/chr10:3406:chr10:123923312-123923145:1;3967/chr16:2924:chr16:68118774-68118605:2;3968/chr1
6:2924:chr16:68118774-68118605:1;3969/chr5:2489:chr5:139228207-139228039:2;3970/chr5:2489:chr5:13922
8207-139228039:1;3971/chr4:2084:chr4:85418790-85418616:2;3972/chr4:2084:chr4:85418790-85418616:1;39
73/chr11:296:chr11:1358628-1358463:2;3974/chr11:296:chr11:1358628-1358463:1;3975/chr16:2668:chr16:580
58788-58058642:2;3976/chr16:2668:chr16:58058788-58058642:1;3977/chr18:585:chr18:32847548-32847403:2;
3978/chr18:585:chr18:32847548-32847403:1;3979/chr8:2113:chr8:91804072-91803904:2;3980/chr8:2113:chr8:
91804072-91803904:1;3981/chr20:924:chr20:32254853-32254720:2;3982/chr20:924:chr20:32254853-3225472
0:1;3983/chr11:98:chr11:627550-627399:2;3984/chr11:98:chr11:627550-627399:1;3985/chrX:537:chrX:250206
43-25020500:2;3986/chrX:537:chrX:25020643-25020500:1;3987/chr9:1188:chr9:99482621-99482456:2;3988/c
hr9:1188:chr9:99482621-99482456:1;3989/chr7:3112:chr7:100091261-100091086:2;3990/chr7:3112:chr7:1000
91261-100091086:1;3991/chr8:3703:chr8:145106353-145106218:2;3992/chr8:3703:chr8:145106353-14510621
8:1;3993/chr15:1830:chr15:83952528-83952400:1;3994/chr10:3406:chr10:123923024-123922851:2;3995/cg06
463958:chr6:166582425-166582252:1;3996/chr18:431:chr18:19751615-19751454:2;3997/chr18:431:chr18:197
51615-19751454:1;3998/chr15:2482:chr15:101268002-101267862:2;3999/chr15:2482:chr15:101268002-10126
7862:1;4000/chr22:1852:chr22:46476495-46476351:2;4001/chr22:1852:chr22:46476495-46476351:1;4002/chr5:
1410:chr5:40682080-40681921:1;4003/chr9:207:chr9:19127574-19127403:2;4004/chr9:207:chr9:19127574-19
127403:1;4005/chr4:1475:chr4:20254251-20254126:2;4006/chr4:1475:chr4:20254251-20254126:1;4007/chr11:
692:chr11:2236920-2236787:1;4008/chr12:1860:chr12:104697560-104697386:2;4009/chr12:1860:chr12:10469
7560-104697386:1;4010/chr2:3074:chr2:114034917-114034742:2;4011/chr2:3049:chr2:112939469-112939295:
2;4012/chr2:3049:chr2:112939469-112939295:1;4013/chr2:3074:chr2:114034917-114034742:1;4014/chr16:247
7:chr16:54971806-54971635:2;4015/chr16:2477:chr16:54971806-54971635:1;4016/chr13:1687:chr13:1136998
98-113699731:2;4017/chr13:1687:chr13:113699898-113699731:1;4018/chr15:1175:chr15:67326173-67326041:
2;4019/chr15:1175:chr15:67326173-67326041:1;4020/chr7:1377:chr7:23287575-23287400:2;4021/chr7:1377:c
hr7:23287575-23287400:1;4022/chr7:1788:chr7:35293516-35293389:2;4023/chr7:1788:chr7:35293516-352933
89:1;4024/chr13:498:chr13:36045534-36045360:2;4025/chr13:498:chr13:36045534-36045360:1;4026/chr2:412
2:chr2:172947969-172947842:2;4027/chr2:4122:chr2:172947969-172947842:1;4028/chr1:4054:chr1:50889086-
50888914:2;4029/chr1:4054:chr1:50889086-50888914:1;4030/chr6:2182:chr6:101850798-101850623:2;4031/c
hr6:2182:chr6:101850798-101850623:1;4032/chr5:2417:chr5:136834315-136834140:2;4033/chr5:2417:chr5:13
6834315-136834140:1;4034/chr12:1757:chr12:95943047-95942874:2;4035/chr12:1757:chr12:95943047-95942
874:1;4036/chr12:1296:chr12:54355324-54355149:2;4037/chr12:1296:chr12:54355324-54355149:1;4038/chr9:
376:chr9:35675764-35675614:2;4039/chr9:376:chr9:35675764-35675614:1;4040/chr14:660:chr14:60981897-60
981742:1;4041/chr19:368:chr19:1469733-1469558:2;4042/chr19:368:chr19:1469733-1469558:1;4043/chr5:192
6:chr5:92906853-92906678:2;4044/chr5:1926:chr5:92906853-92906678:1;4045/chr14:157:chr14:24780178-247
80053:2;4046/chr14:157:chr14:24780178-24780053:1;4047/chr17:1218:chr17:17399342-17399214:2;4048/chr1
7:1218:chr17:17399342-17399214:1;4049/chr4:2332:chr4:122301969-122301809:2;4050/chr4:2332:chr4:12230
1969-122301809:1;4051/cg00662647:chr1:234350127-234349998:2;4052/cg00662647:chr1:234350127-23434
9998:1;4053/chr3:20:chr3:2140840-2140683:2;4054/chr3:20:chr3:2140840-2140683:1;4055/chr2:4451:chr2:200
329102-200328928:2;4056/chr2:4451:chr2:200329102-200328928:1;4057/cg03351460:chr19:19007422-19007
288:2;4058/cg03351460:chr19:19007422-19007288:1;4059/chr12:307:chr12:4919097-4918929:2;4060/chr12:3
07:chr12:4919097-4918929:1;4061/chr8:941:chr8:21647794-21647623:2;4062/chr2:4189:chr2:175202605-1752
02430:2;4063/chr2:4189:chr2:175202605-175202430:1;4064/chr8:941:chr8:21647794-21647623:1;4065/chr4:1
23:chr4:997359-997232:2;4066/chr4:123:chr4:997359-997232:1;4067/chr10:2698:chr10:102586722-102586548:
2;4068/chr10:2698:chr10:102586722-102586548:1;4069/chr7:2143:chr7:49813696-49813547:2;4070/chr7:214
3:chr7:49813696-49813547:1;4071/chr2:4233:chr2:176972686-176972526:2;4072/chr2:4233:chr2:176972686-1
76972526:1;4073/chr19:2730:chr19:31842703-31842529:2;4074/chr19:2730:chr19:31842703-31842529:1;4075/
chr4:773:chr4:5894531-5894359:2;4076/chr4:773:chr4:5894531-5894359:1;4077/chr5:2254:chr5:132161643-1
32161518:2;4078/chr1:4177:chr1:54203679-54203517:2;4079/chr1:4177:chr1:54203679-54203517:1;4080/chr1:
1974:chr1:10698748-10698606:2;4081/chr1:1974:chr1:10698748-10698606:1;4082/chr5:506:chr5:1882419-18
82244:2;4083/chr17:4566:chr17:79919827-79919689:2;4084/chr17:4566:chr17:79919827-79919689:1;4085/chr
22:1308:chr22:41048634-41048459:2;4086/chr7:1497:chr7:27155145-27154971:2;4087/chr15:201:chr15:27017
545-27017418:2;4088/chr15:201:chr15:27017545-27017418:1;4089/chr9:1682:chr9:126135919-126135748:2;4
090/chr9:1682:chr9:126135919-126135748:1;4091/chr1:58:chr1:968445-968288:2;4092/chr1:58:chr1:968445-9
68288:1;4093/chr9:1427:chr9:116226127-116225956:2;4094/chr1:3849:chr1:44872886-44872716:2;4095/chr19:
550:chr19:2251123-2250948:2;4096/chr6:920:chr6:30039324-30039184:2;4097/chr6:920:chr6:30039324-3003
9184:1;4098/chr19:550:chr19:2251123-2250948:1;4099/chr1:1690:chr1:6507591-6507422:2;4100/chr9:1980:ch
r9:130689590-130689421:2;4101/chr19:550:chr19:2250835-2250664:2;4102/chr19:550:chr19:2250835-225066
4:1;4103/chr19:211:chr19:1042155-1041992:2;4104/chr19:211:chr19:1042155-1041992:1;4105/chr8:1962:chr8:
70982199-70982068:2;4106/chr8:1962:chr8:70982199-70982068:1;4107/chr2:117:chr2:946294-946125:2;4108/
chr16:3098:chr16:73100523-73100355:2;4109/chr16:3098:chr16:73100523-73100355:1;4110/chr10:4449:chr10:
133999386-133999214:2;4111/chr14:1137:chr14:77492744-77492577:2;4112/chr14:1137:chr14:77492744-774
92577:1;4113/chr8:3776:chr8:145806718-145806546:2;4114/chr8:3776:chr8:145806718-145806546:1;4115/chr
9:1427:chr9:116226127-116225956:1;4116/chr1:1690:chr1:6507591-6507422:1;4117/chr9:1980:chr9:13068959
0-130689421:1;4118/chr5:1664:chr5:66462587-66462462:2;4119/chr5:1664:chr5:66462587-66462462:1;4120/c
hr10:386:chr10:5734858-5734698:1;4121/chr8:656:chr8:10590236-10590098:2;4122/chr8:656:chr8:10590236-
10590098:1;4123/chr20:2983:chr20:62369570-62369427:2;4124/chr10:2898:chr10:106028588-106028420:2;41
25/chr10:2898:chr10:106028588-106028420:1;4126/chr9:1708:chr9:126777946-126777780:2;4127/chr9:1708:c
hr9:126777946-126777780:1;4128/chr15:523:chr15:37172599-37172470:2;4129/chr15:523:chr15:37172599-37
172470:1;4130/chr10:2949:chr10:108924625-108924467:2;4131/chr10:2949:chr10:108924625-108924467:1;41
32/chr22:1308:chr22:41048634-41048459:1;4133/chr7:1497:chr7:27155145-27154971:1;4134/chr6:95:chr6:137
8677-1378512:2;4135/chr6:95:chr6:1378677-1378512:1;4136/chr2:4187:chr2:175201854-175201680:2;4137/ch
r2:4187:chr2:175201854-175201680:1;4138/chr8:3611:chr8:144810598-144810430:2;4139/chr8:3611:chr8:144
810598-144810430:1;4140/chr5:2417:chr5:136834373-136834199:2;4141/chr5:2417:chr5:136834373-1368341
99:1;4142/chr17:1558:chr17:28088417-28088248:2;4143/chr17:1558:chr17:28088417-28088248:1;4144/chr2:5
386:chr2:239072954-239072779:2;4145/chr8:3309:chr8:143545858-143545729:2;4146/chr8:3309:chr8:143545
858-143545729:1;4147/chr9:1226:chr9:100616660-100616534:2;4148/chr9:1226:chr9:100616660-100616534:1;
4149/chr2:5386:chr2:239072954-239072779:1;4150/chr9:2223:chr9:133540401-133540235:2;4151/chr9:2223:
chr9:133540401-133540235:1;4152/chr13:45:chr13:20176518-20176343:2;4153/chr13:1184:chr13:109793302-
109793174:2;4154/chr13:1184:chr13:109793302-109793174:1;4155/chr11:4678:chr11:130029583-130029419:
2;4156/chr11:4678:chr11:130029583-130029419:1;4157/chr13:45:chr13:20176518-20176343:1;4158/chr19:367:
chr19:1467595-1467427:2;4159/chr19:367:chr19:1467595-1467427:1;4160/chr13:877:chr13:79183568-791833
98:2;4161/chr13:877:chr13:79183568-79183398:1;4162/chr6:3358:chr6:168502101-168501934:2;4163/chr6:33
58:chr6:168502101-168501934:1;4164/chr15:2393:chr15:99645392-99645218:2;4165/chr15:2393:chr15:99645
392-99645218:1;4166/chr4:2973:chr4:188916656-188916483:2;4167/chr4:2973:chr4:188916656-188916483:1;
4168/chr5:1679:chr5:68711138-68710972:2;4169/chr5:1679:chr5:68711138-68710972:1;4170/chr5:2254:chr5:1
32161643-132161518:1;4171/chr18:77:chr18:4455636-4455486:2;4172/chr18:77:chr18:4455636-4455486:1;41
73/chr7:4786:chr7:157406102-157405935:2;4174/chr7:4786:chr7:157406102-157405935:1;4175/chr9:239:chr9:
23821953-23821816:2;4176/chr9:239:chr9:23821953-23821816:1;4177/chr6:105:chr6:1393307-1393144:2;417
8/chr6:105:chr6:1393307-1393144:1;4179/chrX:1560:chrX:133684982-133684808:2;4180/chr1:7360:chr1:2469
52603-246952452:2;4181/chr1:7360:chr1:246952603-246952452:1;4182/chr18:97:chr18:5630029-5629868:2;4
183/chr18:97:chr18:5630029-5629868:1;4184/chr22:1873:chr22:46500126-46499997:2;4185/chr22:1873:chr22:
46500126-46499997:1;4186/chr14:626:chr14:59931635-59931510:2;4187/chr14:626:chr14:59931635-5993151
0:1;4188/chr4:1475:chr4:20254329-20254159:2;4189/chr4:1475:chr4:20254329-20254159:1;4190/chr15:264:ch
r15:28352656-28352482:2;4191/chr15:264:chr15:28352656-28352482:1;4192/chr22:2205:chr22:48970972-489
70828:2;4193/chr22:2205:chr22:48970972-48970828:1;4194/chr14:2038:chr14:101923831-101923660:2;4195/
chr14:2038:chr14:101923831-101923660:1;4196/chr2:5420:chr2:239755724-239755578:1;4197/chr15:203:chr1
5:27018638-27018470:2;4198/chr15:203:chr15:27018638-27018470:1;4199/chr12:1319:chr12:54427781-54427
637:2;4200/chr19:4937:chr19:58868006-58867833:2;4201/chr19:4937:chr19:58868006-58867833:1;4202/chr9:
2443:chr9:135462247-135462117:2;4203/chr9:2443:chr9:135462247-135462117:1;4204/chr15:2003:chr15:899
22235-89922066:2;4205/chr15:2003:chr15:89922235-89922066:1;4206/chr6:2142:chr6:100061733-100061608:
2;4207/chr6:2142:chr6:100061733-100061608:1;4208/chr16:3585:chr16:85320838-85320668:2;4209/chr16:358
5:chr16:85320838-85320668:1;4210/chr10:1049:chr10:30024824-30024693:2;4211/chr15:1175:chr15:6732612
6-67325980:1;4212/chr10:1049:chr10:30024824-30024693:1;4213/chr1:3957:chr1:47905286-47905124:2;4214/
chr1:3957:chr1:47905286-47905124:1;4215/chr10:2740:chr10:102900030-102899855:2;4216/chr10:2740:chr10:
102900030-102899855:1;4217/chr8:1107:chr8:24771275-24771100:2;4218/chr8:1107:chr8:24771275-2477110
0:1;4219/chr20:524:chr20:21376562-21376424:2;4220/chr20:524:chr20:21376562-21376424:1;4221/chr8:2786:
chr8:140716233-140716058:2;4222/chr8:2786:chr8:140716233-140716058:1;4223/chr13:865:chr13:79170184-
79170016:2;4224/chr13:865:chr13:79170184-79170016:1;4225/chr12:1084:chr12:50297660-50297510:2;4226/
chr16:241:chr16:1040972-1040802:2;4227/chr16:241:chr16:1040972-1040802:1;4228/chr19:4654:chr19:55598
560-55598429:1;4229/chr20:1816:chr20:55206015-55205849:2;4230/chr19:2032:chr19:17439780-17439654:2;
4231/chr20:1816:chr20:55206015-55205849:1;4232/chrX:27:chrX:334573-334430:2;4233/chrX:27:chrX:334573-
334430:1;4234/chr17:3474:chr17:73585037-73584909:2;4235/chr17:3474:chr17:73585037-73584909:1;4236/c
hrX:132:chrX:933818-933647:1;4237/chr15:839:chr15:52404890-52404719:2;4238/chr15:839:chr15:52404890-
52404719:1;4239/chr12:1542:chr12:66123479-66123351:2;4240/chr12:1542:chr12:66123479-66123351:1;4241/
chr13:917:chr13:88324731-88324561:2;4242/chr13:917:chr13:88324731-88324561:1;4243/chr14:1768:chr14:9
9712787-99712644:2;4244/chr14:1768:chr14:99712787-99712644:1;4245/cg27014135:chr16:25703603-25703
478:2;4246/cg27014135:chr16:25703603-25703478:1;4247/cg04140862:chr14:70654701-70654533:1;4248/chr
11:4527:chr11:126286809-126286638:2;4249/chr11:4527:chr11:126286809-126286638:1;4250/chr19:4937:chr
19:58868081-58867930:2;4251/chr19:4937:chr19:58868081-58867930:1;4252/chr19:362:chr19:1457241-14570
70:2;4253/chr19:362:chr19:1457241-1457070:1;4254/chr3:2636:chr3:138664772-138664647:2;4255/chr3:2636:
chr3:138664772-138664647:1;4256/chr20:1728:chr20:50721874-50721710:2;4257/chr20:1728:chr20:50721874-
50721710:1;4258/chr9:3508:chr9:139740890-139740715:2;4259/chr9:3508:chr9:139740890-139740715:1;426
0/cg07748540:chr11:104034622-104034493:1;4261/chr19:1417:chr19:9473753-9473594:2;4262/chr18:891:chr
18:47795096-47794921:2;4263/chr12:3052:chr12:130389012-130388837:2;4264/chr10:3710:chr10:128077448-
128077273:2;4265/chr10:3710:chr10:128077448-128077273:1;4266/chr2:2852:chr2:107503402-107503235:2;4
267/chr2:2852:chr2:107503402-107503235:1;4268/chr1:4372:chr1:63790339-63790166:2;4269/chr1:4372:chr1:
63790339-63790166:1;4270/chr11:2774:chr11:68145669-68145544:1;4271/chr19:3497:chr19:41106090-41105
919:2;4272/chr19:3497:chr19:41106090-41105919:1;4273/chr5:506:chr5:1882479-1882307:2;4274/chr5:506:ch
r5:1882479-1882307:1;4275/chr4:1727:chr4:44450672-44450505:2;4276/chr4:1727:chr4:44450672-44450505:
1;4277/chr1:645:chr1:2706659-2706490:2;4278/chr1:645:chr1:2706659-2706490:1;4279/chr7:4109:chr7:15065
5373-150655202:2;4280/chr7:4109:chr7:150655373-150655202:1;4281/chr2:259:chr2:1748097-1747925:2;428
2/chr2:259:chr2:1748097-1747925:1;4283/chr19:1417:chr19:9473753-9473594:1;4284/chr12:1447:chr12:58013
641-58013472:2;4285/chr12:1447:chr12:58013641-58013472:1;4286/chr4:759:chr4:5710373-5710248:2;4287/c
hr4:759:chr4:5710373-5710248:1;4288/chr5:504:chr5:1878678-1878542:2;4289/chr5:504:chr5:1878678-18785
42:1;4290/chr5:705:chr5:2751859-2751722:2;4291/chr5:705:chr5:2751859-2751722:1;4292/chr19:56:chr19:518
996-518826:2;4293/chr19:56:chr19:518996-518826:1;4294/chr15:219:chr15:27216913-27216787:2;4295/chr15:
219:chr15:27216913-27216787:1;4296/chr9:3751:chr9:140681076-140680950:1;4297/chr17:2778:chr17:58227
861-58227695:2;4298/chr17:2778:chr17:58227861-58227695:1;4299/chr8:1720:chr8:54792258-54792133:2;43
00/chr2:4217:chr2:176947321-176947190:2;4301/chr11:2492:chr11:65313988-65313813:2;4302/chr11:2492:ch
r11:65313988-65313813:1;4303/chr7:547:chr7:1577120-1576948:2;4304/chr7:547:chr7:1577120-1576948:1;43
05/chr11:2492:chr11:65314345-65314220:2;4306/chr11:2492:chr11:65314345-65314220:1;4307/chr21:100:chr
21:27945575-27945450:2;4308/chr21:100:chr21:27945575-27945450:1;4309/chr2:4131:chr2:172965219-17296
5045:2;4310/chr2:4131:chr2:172965219-172965045:1;4311/chr15:2063:chr15:90543525-90543355:2;4312/chr1
5:2063:chr15:90543525-90543355:1;4313/chr10:1958:chr10:73848037-73847885:2;4314/chr10:1958:chr10:738
48037-73847885:1;4315/chr8:3703:chr8:145106757-145106631:2;4316/chr8:3703:chr8:145106757-145106631:
1;4317/chr6:2232:chr6:107955926-107955751:2;4318/chr6:2232:chr6:107955926-107955751:1;4319/chr5:2254:
chr5:132161373-132161217:2;4320/chr5:2254:chr5:132161373-132161217:1;4321/chr11:692:chr11:2237011-2
236846:2;4322/chr11:692:chr11:2237011-2236846:1;4323/chr12:1282:chr12:54345739-54345565:2;4324/chr17:
4139:chr17:77788791-77788627:2;4325/chr11:4765:chr11:131780660-131780487:2;4326/chr5:3129:chr5:1708
48128-170847954:2;4327/chr17:3474:chr17:73584997-73584831:2;4328/chr8:3759:chr8:145698283-14569813
4:2;4329/chr8:3759:chr8:145698283-145698134:1;4330/chr10:4868:chr10:135016006-135015832:2;4331/chr10:
4868:chr10:135016006-135015832:1;4332/chrX:810:chrX:48685128-48684999:2;4333/chrX:810:chrX:4868512
8-48684999:1;4334/chr10:2407:chr10:94451670-94451507:2;4335/chr10:2407:chr10:94451670-94451507:1;43
36/chr17:1855:chr17:36666713-36666556:2;4337/chr17:1855:chr17:36666713-36666556:1;4338/chr10:784:chr
10:16562252-16562097:2;4339/chr10:784:chr10:16562252-16562097:1;4340/chr15:523:chr15:37172870-37172
725:2;4341/chr15:523:chr15:37172870-37172725:1;4342/chr18:994:chr18:55095432-55095276:2;4343/chr18:9
94:chr18:55095432-55095276:1;4344/chr16:4109:chr16:88497443-88497272:2;4345/chr16:4109:chr16:884974
43-88497272:1;4346/chr10:1958:chr10:73848257-73848107:2;4347/chr10:1958:chr10:73848257-73848107:1;4
348/chr11:4458:chr11:125036819-125036669:2;4349/chr11:4458:chr11:125036819-125036669:1;4350/chr10:8
72:chr10:22634346-22634171:2;4351/chr10:872:chr10:22634346-22634171:1;4352/chr6:95:chr6:1378905-1378
750:2;4353/chr6:95:chr6:1378905-1378750:1;4354/chr18:763:chr18:44337107-44336975:2;4355/chr22:141:chr
22:19512078-19511945:2;4356/chr7:1788:chr7:35293910-35293746:2;4357/chr7:1788:chr7:35293910-3529374
6:1;4358/chr8:1752:chr8:56014853-56014711:2;4359/chr8:1752:chr8:56014853-56014711:1;4360/chr4:122:chr
4:996415-996264:2;4361/chr4:122:chr4:996415-996264:1;4362/chr3:160:chr3:9595432-9595263:2;4363/chr3:1
60:chr3:9595432-9595263:1;4364/chr13:870:chr13:79176643-79176476:2;4365/chr13:870:chr13:79176643-791
76476:1;4366/chr8:738:chr8:11560111-11559974:2;4367/chr8:738:chr8:11560111-11559974:1;4368/chr2:1902:
chr2:63275289-63275122:1;4369/chr22:1873:chr22:46500395-46500230:2;4370/chr22:1873:chr22:46500395-4
6500230:1;4371/chr18:91:chr18:5543489-5543323:2;4372/chr18:91:chr18:5543489-5543323:1;4373/chr13:368:
chr13:28674683-28674526:2;4374/chr13:368:chr13:28674683-28674526:1;4375/chr22:1094:chr22:38478869-3
8478699:2;4376/chr22:1094:chr22:38478869-38478699:1;4377/chr16:2882:chr16:67428601-67428436:2;4378/
chr16:2882:chr16:67428601-67428436:1;4379/chr10:1271:chr10:43762434-43762276:2;4380/chr17:274:chr17:
1960324-1960165:2;4381/chr17:274:chr17:1960324-1960165:1;4382/chr10:1271:chr10:43762434-43762276:1;
4383/chr17:1676:chr17:32484355-32484180:1;4384/chr10:2138:chr10:81002084-81001915:1;4385/chr9:1715:c
hr9:126780132-126780007:2;4386/chr9:1715:chr9:126780132-126780007:1;4387/chr17:276:chr17:1961109-19
60963:2;4388/chr17:276:chr17:1961109-1960963:1;4389/chr15:1841:chr15:84322497-84322322:2;4390/chr15:
1841:chr15:84322497-84322322:1;4391/chr1:1852:chr1:8277864-8277739:1;4392/chr13:1076:chr13:10064206
8-100641943:2;4393/chr2:1346:chr2:27530914-27530740:2;4394/chr2:1346:chr2:27530914-27530740:1;4395/c
hr19:1749:chr19:14090089-14089915:1;4396/chr2:3108:chr2:115919729-115919583:2;4397/chr2:3108:chr2:11
5919729-115919583:1;4398/cg19763461:chr2:63284580-63284411:1;4399/chr6:21:chr6:392067-391900:2;440
0/chr6:21:chr6:392067-391900:1;4401/chr19:918:chr19:4328973-4328812:1;4402/chr14:1402:chr14:92789961-
92789812:2;4403/chr14:1402:chr14:92789961-92789812:1;4404/chr2:2852:chr2:107503513-107503344:2;4405/
chr4:616:chr4:3734447-3734285:2;4406/chr4:616:chr4:3734447-3734285:1;4407/chr18:966:chr18:53447862-5
3447705:2;4408/chr18:966:chr18:53447862-53447705:1;4409/chr2:1902:chr2:63274958-63274785:2;4410/chr1
3:1196:chr13:110437586-110437432:2;4411/chr16:2882:chr16:67428372-67428219:2;4412/chr16:2882:chr16:6
7428372-67428219:1;4413/chr17:2816:chr17:59488655-59488492:2;4414/chr17:2816:chr17:59488655-594884
92:1;4415/chr1:252:chr1:1565660-1565491:2;4416/chr1:252:chr1:1565660-1565491:1;4417/chr17:1855:chr17:3
6667234-36667073:2;4418/chr17:1855:chr17:36667234-36667073:1;4419/chr1:6194:chr1:203044828-2030446
77:1;4420/chr2:1902:chr2:63274958-63274785:1;4421/chr9:1226:chr9:100616968-100616796:2;4422/chr9:122
6:chr9:100616968-100616796:1;4423/chr19:567:chr19:2291321-2291158:2;4424/chr19:567:chr19:2291321-229
1158:1;4425/chr19:1699:chr19:13215542-13215370:2;4426/chr1:3539:chr1:37500019-37499883:2;4427/chr1:3
539:chr1:37500019-37499883:1;4428/chr15:1832:chr15:83953323-83953184:2;4429/chr15:1832:chr15:839533
23-83953184:1;4430/chr1:5882:chr1:180202555-180202383:2;4431/chr1:5882:chr1:180202555-180202383:1;4
432/chr19:4004:chr19:48984036-48983865:2;4433/chr19:4004:chr19:48984036-48983865:1;4434/chr2:2852:ch
r2:107503513-107503344:1;4435/chr3:1771:chr3:73673807-73673638:2;4436/chr3:1771:chr3:73673807-73673
638:1;4437/chr8:3759:chr8:145698518-145698393:2;4438/chr8:3759:chr8:145698518-145698393:1;4439/chr3:
220:chr3:10858102-10857954:2;4440/chr3:220:chr3:10858102-10857954:1;4441/chr2:1230:chr2:25500371-255
00226:2;4442/chr2:1230:chr2:25500371-25500226:1;4443/chr17:859:chr17:8926884-8926716:2;4444/chr17:85
9:chr17:8926884-8926716:1;4445/chr2:3999:chr2:162283734-162283570:2;4446/chr2:3999:chr2:162283734-16
2283570:1;4447/chr7:4109:chr7:150655712-150655585:2;4448/chr7:4109:chr7:150655712-150655585:1;4449/
chr5:3116:chr5:170735731-170735573:2;4450/chr5:3116:chr5:170735731-170735573:1;4451/chr11:2667:chr11:
67290793-67290618:1;4452/chr13:519:chr13:37006315-37006146:2;4453/chr13:519:chr13:37006315-3700614
6:1;4454/chr20:16:chr20:591041-590883:2;4455/chr20:16:chr20:591041-590883:1;4456/chr19:1417:chr19:9473
704-9473529:2;4457/chr19:1417:chr19:9473704-9473529:1;4458/chr10:1049:chr10:30024300-30024127:2;445
9/chr10:1049:chr10:30024300-30024127:1;4460/chr14:288:chr14:34419856-34419690:1;4461/chr4:1277:chr4:8
873874-8873737:2;4462/chr4:1277:chr4:8873874-8873737:1;4463/chr3:2768:chr3:147127729-147127565:2;44
64/chr3:2768:chr3:147127729-147127565:1;4465/chr12:3619:chr12:133485256-133485127:2;4466/chr12:3619:
chr12:133485256-133485127:1;4467/chr11:141:chr11:849586-849411:2;4468/chr6:2135:chr6:100051256-1000
51103:1;4469/chr2:605:chr2:5837141-5836990:2;4470/chr2:605:chr2:5837141-5836990:1;4471/chr18:1125:chr
18:63418046-63417893:2;4472/chr18:1125:chr18:63418046-63417893:1;4473/chr2:1926:chr2:63286872-63286
719:2;4474/chr2:1926:chr2:63286872-63286719:1;4475/chr20:732:chr20:25129187-25129014:2;4476/chr20:73
2:chr20:25129187-25129014:1;4477/chr5:2354:chr5:134825921-134825774:2;4478/chr17:273:chr17:1959837-1
959683:2;4479/chr17:273:chr17:1959837-1959683:1;4480/chr12:3095:chr12:130647294-130647166:2;4481/chr
12:3095:chr12:130647294-130647166:1;4482/chr13:982:chr13:96294431-96294279:2;4483/chr13:982:chr13:96
294431-96294279:1;4484/chr7:2143:chr7:49813881-49813756:2;4485/chr7:2143:chr7:49813881-49813756:1;4
486/chr17:965:chr17:10102122-10101966:2;4487/chr17:965:chr17:10102122-10101966:1;4488/chr21:1182:chr
21:46848236-46848064:2;4489/chr21:1182:chr21:46848236-46848064:1;4490/chr8:263:chr8:1444072-1443908:
2;4491/chr8:263:chr8:1444072-1443908:1;4492/chr21:1265:chr21:47062764-47062591:2;4493/chr21:1265:chr
21:47062764-47062591:1;4494/chr1:691:chr1:2777213-2777070:2;4495/chr18:566:chr18:31020892-31020737:
2;4496/chr18:566:chr18:31020892-31020737:1;4497/chr20:534:chr20:21491454-21491279:2;4498/chr20:534:c
hr20:21491454-21491279:1;4499/chr3:20:chr3:2140928-2140801:2;4500/chr3:20:chr3:2140928-2140801:1;450
1/chr7:2696:chr7:72848500-72848343:2;4502/chr1:3659:chr1:39957400-39957262:2;4503/chr1:3659:chr1:3995
7400-39957262:1;4504/chr20:2966:chr20:62272898-62272769:2;4505/chr5:2198:chr5:127874800-127874673:2;
4506/chr5:2198:chr5:127874800-127874673:1;4507/chr19:2032:chr19:17440035-17439905:2;4508/chr19:2032:
chr19:17440035-17439905:1;4509/chr7:1422:chr7:25892622-25892470:2;4510/chr7:1422:chr7:25892622-2589
2470:1;4511/chr21:1253:chr21:47010268-47010093:2;4512/chr21:158:chr21:32931742-32931578:2;4513/chr21:
158:chr21:32931742-32931578:1;4514/chr16:241:chr16:1041202-1041028:2;4515/chr22:2676:chr22:50496653-
50496512:2;4516/chr22:2676:chr22:50496653-50496512:1;4517/chr18:723:chr18:43913977-43913812:2;4518/
chr18:723:chr18:43913977-43913812:1;4519/chr3:2629:chr3:138658509-138658375:2;4520/chr3:2629:chr3:13
8658509-138658375:1;4521/chr7:3399:chr7:112430518-112430343:2;4522/chr7:3399:chr7:112430518-112430
343:1;4523/cg12587766:chr5:38556497-38556325:2;4524/chr15:2003:chr15:89922329-89922154:2;4525/chr15:
2003:chr15:89922329-89922154:1;4526/chr10:629:chr10:11784556-11784407:2;4527/chr10:629:chr10:117845
56-11784407:1;4528/chr2:2227:chr2:74743203-74743048:2;4529/chr3:2385:chr3:128212280-128212149:2;453
0/chr1:4486:chr1:77334071-77333897:1;4531/chr12:1296:chr12:54355591-54355426:2;4532/chr17:2375:chr17:
46711253-46711089:2;4533/chr17:2375:chr17:46711253-46711089:1;4534/chr12:1296:chr12:54355591-54355
426:1;4535/chr10:4591:chr10:134601877-134601703:2;4536/chr10:4591:chr10:134601877-134601703:1;4537/
chr6:1972:chr6:84418834-84418700:2;4538/chr6:1972:chr6:84418834-84418700:1;4539/chr19:211:chr19:1042
032-1041857:2;4540/chr11:2224:chr11:62212202-62212039:2;4541/chr11:2224:chr11:62212202-62212039:1;4
542/chr8:3703:chr8:145106475-145106305:2;4543/chr8:3703:chr8:145106475-145106305:1;4544/chr15:2063:c
hr15:90543455-90543308:1;4545/chr22:2705:chr22:50629112-50628937:1;4546/chr17:1078:chr17:12877734-1
2877568:2;4547/chr17:1078:chr17:12877734-12877568:1;4548/chr8:1695:chr8:53852581-53852433:2;4549/chr
8:1695:chr8:53852581-53852433:1;4550/chr9:3497:chr9:139716160-139716015:2;4551/chr9:3497:chr9:139716
160-139716015:1;4552/chr13:386:chr13:29107331-29107195:2;4553/chr13:386:chr13:29107331-29107195:1;4
554/cg11666087:chr1:49242637-49242497:2;4555/cg16573178:chr1:49242637-49242497:2;4556/cg11666087:
chr1:49242637-49242497:1;4557/cg16573178:chr1:49242637-49242497:1;4558/chr19:4808:chr19:57050435-5
7050286:2;4559/chr19:4808:chr19:57050435-57050286:1;4560/chr10:958:chr10:26680817-26680643:2;4561/c
hr10:958:chr10:26680817-26680643:1;4562/chr18:1044:chr18:56940333-56940164:2;4563/chr18:1044:chr18:5
6940333-56940164:1;4564/chr13:982:chr13:96294354-96294224:2;4565/chr13:982:chr13:96294354-96294224:
1;4566/chr10:3225:chr10:119305108-119304933:2;4567/chr10:3225:chr10:119305108-119304933:1;4568/chr7:
5168:chr7:158937611-158937438:2;4569/chr7:5168:chr7:158937611-158937438:1;4570/chr12:1125:chr12:522
15612-52215466:2;4571/chr12:1125:chr12:52215612-52215466:1;4572/chr22:2791:chr22:51136022-51135849:
2;4573/chr22:2791:chr22:51136022-51135849:1;4574/cg12587766:chr5:38556497-38556325:1;4575/chr17:350
2:chr17:73749729-73749597:2;4576/chr17:3502:chr17:73749729-73749597:1;4577/chr2:2227:chr2:74743203-7
4743048:1;4578/chr3:2385:chr3:128212280-128212149:1;4579/chr10:4583:chr10:134599116-134598942:2;458
0/chr4:24:chr4:568792-568654:2;4581/chr4:24:chr4:568792-568654:1;4582/chr10:4583:chr10:134599116-1345
98942:1;4583/chr15:1832:chr15:83953571-83953400:2;4584/chr15:1832:chr15:83953571-83953400:1;4585/chr
7:4724:chr7:157072252-157072088:2;4586/chr7:4724:chr7:157072252-157072088:1;4587/chr19:211:chr19:104
2032-1041857:1;4588/chr8:2423:chr8:120220685-120220558:2;4589/chr8:2423:chr8:120220685-120220558:1;
4590/chr16:2915:chr16:67918758-67918597:1;4591/chr4:470:chr4:2820480-2820317:2;4592/chr4:470:chr4:282
0480-2820317:1;4593/chr14:1434:chr14:93154181-93154008:1;4594/chr7:1376:chr7:23245809-23245651:2;45
95/chr7:1376:chr7:23245809-23245651:1;4596/chr13:452:chr13:31480569-31480437:2;4597/chr13:452:chr13:3
1480569-31480437:1;4598/chr7:2162:chr7:50343793-50343654:2;4599/chr7:2162:chr7:50343793-50343654:1;
4600/chr2:3718:chr2:131797625-131797475:2;4601/chr2:3718:chr2:131797625-131797475:1;4602/chr17:1243:
chr17:17626139-17625965:2;4603/chr17:1243:chr17:17626139-17625965:1;4604/chr17:2417:chr17:47073452-
47073308:2;4605/chr17:2417:chr17:47073452-47073308:1;4606/chr17:176:chr17:1173876-1173729:2;4607/chr
17:176:chr17:1173876-1173729:1;4608/chr15:937:chr15:58358425-58358264:2;4609/chr15:937:chr15:5835842
5-58358264:1;4610/chr5:2392:chr5:135266577-135266405:2;4611/chr5:2392:chr5:135266577-135266405:1;46
12/chr17:4511:chr17:79615177-79615039:2;4613/chr17:4511:chr17:79615177-79615039:1;4614/chr5:2354:chr
5:134825921-134825774:1;4615/chr22:1790:chr22:46263482-46263324:2;4616/chr22:1790:chr22:46263482-46
263324:1;4617/chr22:2703:chr22:50623333-50623190:2;4618/chr22:2703:chr22:50623333-50623190:1;4619/c
hr13:1471:chr13:112759139-112758994:2;4620/chr13:1471:chr13:112759139-112758994:1;4621/chr15:1538:c
hr15:76630446-76630274:2;4622/chr15:1538:chr15:76630446-76630274:1;4623/chr7:2148:chr7:49815373-498
15201:2;4624/chr7:2148:chr7:49815373-49815201:1;4625/chr4:1272:chr4:8863792-8863664:2;4626/chr1:1408:
chr1:4715522-4715396:2;4627/chr1:1408:chr1:4715522-4715396:1;4628/chr4:1272:chr4:8863792-8863664:1;4
629/chr1:691:chr1:2777213-2777070:1;4630/chr16:2445:chr16:54324352-54324220:2;4631/chr16:2445:chr16:5
4324352-54324220:1;4632/chr15:1205:chr15:68122181-68122035:2;4633/chr13:714:chr13:53421595-5342143
9:2;4634/chr13:714:chr13:53421595-53421439:1;4635/chr7:2696:chr7:72848500-72848343:1;4636/chr8:1971:c
hr8:72460165-72459997:2;4637/chr20:2966:chr20:62272898-62272769:1;4638/chr8:1971:chr8:72460165-7245
9997:1;4639/chr2:5038:chr2:233285674-233285528:2;4640/chr2:5038:chr2:233285674-233285528:1;4641/chr1
7:176:chr17:1174236-1174062:2;4642/chr17:176:chr17:1174236-1174062:1;4643/chr15:1205:chr15:68122181-
68122035:1;4644/chr21:1253:chr21:47010268-47010093:1;4645/chr10:1066:chr10:30348021-30347848:2;4646/
chr10:1066:chr10:30348021-30347848:1;4647/chr17:2823:chr17:59529538-59529363:2;4648/chr17:2823:chr1
7:59529538-59529363:1;4649/chr8:1276:chr8:31498016-31497870:2;4650/chr8:1276:chr8:31498016-31497870:
1;4651/chr16:241:chr16:1041202-1041028:1;4652/chr2:4189:chr2:175202527-175202385:2;4653/chr2:4189:ch
r2:175202527-175202385:1;4654/chr5:1710:chr5:72594396-72594231:2;4655/chr5:1710:chr5:72594396-72594
231:1;4656/chr10:3131:chr10:118030795-118030641:2;4657/chr10:3131:chr10:118030795-118030641:1;4658/
chr3:1095:chr3:47051083-47050944:2;4659/chr3:1095:chr3:47051083-47050944:1;4660/chr10:1053:chr10:300
25956-30025824:2;4661/chr10:1053:chr10:30025956-30025824:1;4662/chr22:1823:chr22:46368462-46368287:
2;4663/chr22:1823:chr22:46368462-46368287:1;4664/chr22:162:chr22:19711633-19711462:2;4665/chr22:162:
chr22:19711633-19711462:1;4666/chr10:950:chr10:26223491-26223364:2;4667/chr10:950:chr10:26223491-26
223364:1;4668/chr1:1739:chr1:7022139-7021971:1;4669/chr8:3689:chr8:145052136-145052006:2;4670/chr8:3
689:chr8:145052136-145052006:1;4671/cg27200446:chr6:41606486-41606315:2;4672/cg27200446:chr6:4160
6486-41606315:1;4673/chr2:4739:chr2:219866343-219866217:2;4674/chr2:4739:chr2:219866343-219866217:1;
4675/chr10:958:chr10:26680894-26680726:2;4676/chr10:524:chr10:8097801-8097629:2;4677/chr10:524:chr10:
8097801-8097629:1;4678/chr10:958:chr10:26680894-26680726:1;4679/chr14:847:chr14:69095246-69095093:
2;4680/chr14:847:chr14:69095246-69095093:1;4681/chr10:2667:chr10:102475579-102475404:2;4682/chr10:26
67:chr10:102475579-102475404:1;4683/chr12:1112:chr12:51717924-51717788:2;4684/chr12:1112:chr12:5171
7924-51717788:1;4685/chr14:2078:chr14:102027682-102027508:2;4686/chr14:2078:chr14:102027682-102027
508:1;4687/chr1:604:chr1:2461934-2461762:2;4688/chr1:604:chr1:2461934-2461762:1;4689/chr5:1926:chr5:92
906789-92906627:2;4690/chr5:1926:chr5:92906789-92906627:1;4691/chr4:2703:chr4:172734525-172734362:2;
4692/chr4:2703:chr4:172734525-172734362:1;4693/cg01090834:chr21:38081202-38081050:2;4694/cg010908
34:chr21:38081202-38081050:1;4695/chr2:3997:chr2:162280627-162280452:2;4696/chr2:3997:chr2:16228062
7-162280452:1;4697/chr6:705:chi6:2751212-2751038:2;4698/chr5:705:chr6:2751212-2751038:1;4699/chr15:18
42:chr15:84322823-84322685:2;4700/chr15:1842:chr15:84322823-84322685:1;4701/chr15:207:chr15:2711237
0-27112216:2;4702/chr15:207:chr15:27112370-27112216:1;4703/chr20:1812:chr20:55202313-55202138:2;470
4/chr20:1812:chr20:55202313-55202138:1;4705/chr5:1710:chr5:72594236-72594071:2;4706/chr5:1710:chr5:72
594236-72594071:1;4707/chr18:994:chr18:55094947-55094772:2;4708/chr18:994:chr18:55094947-55094772:1;
4709/chr10:4591:chr10:134601955-134601784:2;4710/chr10:4591:chr10:134601955-134601784:1;4711/chr22:
2149:chr22:48886069-48885943:2;4712/chr22:2149:chr22:48886069-48885943:1;4713/chr2:3000:chr2:110372
462-110372324:2;4714/chr2:3000:chr2:110372462-110372324:1;4715/chr17:2797:chr17:59473560-59473410:2;
4716/chr17:2797:chr17:59473560-59473410:1;4717/chr8:1033:chr8:22548409-22548240:2;4718/chr8:1033:chr
8:22548409-22548240:1;4719/chr12:1460:chr12:58129983-58129822:2;4720/chr12:1460:chr12:58129983-5812
9822:1;4721/chr5:3858:chr5:179244344-179244173:2;4722/chr5:3858:chr5:179244344-179244173:1;4723/chr1
2:1757:chr12:95941963-95941829:2;4724/chr12:1757:chr12:95941963-95941829:1;4725/chr7:3501:chr7:12767
1993-127671859:2;4726/chr7:3501:chr7:127671993-127671859:1;4727/chr19:830:chr19:3786599-3786443:2;4
728/chr19:830:chr19:3786599-3786443:1;4729/chr1:2371:chr1:16085635-16085460:2;4730/chr1:2371:chr1:160
85635-16085460:1;4731/chr2:5206:chr2:236579795-236579650:2;4732/chr2:5206:chr2:236579795-236579650:
1;4733/chr17:3474:chr17:73584687-73584518:2;4734/chr17:3474:chr17:73584687-73584518:1;4735/chr1:3659:
chr1:39957785-39957619:2;4736/chr1:3659:chr1:39957785-39957619:1;4737/chr12:1282:chr12:54345739-543
45565:1;4738/chr1:1690:chr1:6507456-6507305:2;4739/chr1:1690:chr1:6507456-6507305:1;4740/chr9:2117:ch
r9:132382687-132382516:2;4741/chr9:2117:chr9:132382687-132382516:1;4742/chr17:4139:chr17:77788791-7
7788627:1;4743/chr11:4765:chr11:131780660-131780487:1;4744/chr5:3129:chr5:170848128-170847954:1;474
5/chr17:3474:chr17:73584997-73584831:1;4746/chr5:3649:chr5:178017056-178016917:2;4747/chr5:3649:chr5:
178017056-178016917:1;4748/chr2:4224:chr2:176957930-176957781:2;4749/chr2:4224:chr2:176957930-1769
57781:1;4750/chr18:763:chr18:44337107-44336975:1;4751/chr22:141:chr22:19512078-19511945:1;4752/chr7:
1085:chr7:4923093-4922918:2;4753/chr7:1085:chr7:4923093-4922918:1;4754/chr14:2165:chr14:103395683-10
3395530:2;4755/chr14:2165:chr14:103395683-103395530:1;4756/chr17:275:chr17:1960610-1960450:2;4757/c
hr17:275:chr17:1960610-1960450:1;4758/chr4:1405:chr4:13545653-13545494:2;4759/chr4:1405:chr4:1354565
3-13545494:1;4760/chr4:1270:chr4:8863147-8862993:2;4761/chr4:1270:chr4:8863147-8862993:1;4762/chr13:1
076:chr13:100642068-100641943:1;4763/chr8:766:chr8:12809289-12809164:2;4764/chr8:766:chr8:12809289-1
2809164:1;4765/chr15:219:chr15:27216433-27216299:2;4766/chr15:219:chr15:27216433-27216299:1;4767/chr
14:2166:chr14:103395985-103395810:2;4768/chr14:2166:chr14:103395985-103395810:1;4769/chr12:1757:chr
12:95942489-95942317:2;4770/chr12:1757:chr12:95942489-95942317:1;4771/chr1:3709:chr1:41327658-41327
484:2;4772/chr12:428:chr12:6665399-6665270:2;4773/chr12:428:chr12:6665399-6665270:1;4774/chr10:4269:c
hr10:133109835-133109670:2;4775/chr3:2880:chr3:156544239-156544108:2;4776/chr3:2880:chr3:156544239-
156544108:1;4777/chr13:1196:chr13:110437586-110437432:1;4778/chr10:4269:chr10:133109835-133109670:
1;4779/chr12:438:chr12:6883091-6882918:2;4780/chr12:438:chr12:6883091-6882918:1;4781/chr22:1288:chr22:
40391298-40391142:2;4782/chr22:1288:chr22:40391298-40391142:1;4783/chr14:157:chr14:24780336-247801
66:2;4784/chr14:157:chr14:24780336-24780166:1;4785/chr7:4820:chr7:157481795-157481628:2;4786/chr7:48
20:chr7:157481795-157481628:1;4787/chr19:1699:chr19:13215542-13215370:1;4788/chr15:203:chr15:270187
82-27018607:2;4789/chr15:203:chr15:27018782-27018607:1;4790/chr19:2730:chr19:31842757-31842604:2;47
91/chr19:2730:chr19:31842757-31842604:1;4792/chrX:810:chrX:48685429-48685279:2;4793/chrX:810:chrX:48
685429-48685279:1;4794/chr8:565:chr8:8559711-8559586:2;4795/chr8:565:chr8:8559711-8559586:1;4796/chr
1:3709:chr1:41327658-41327484:1;4797/chr5:3347:chr5:175224294-175224159:2;4798/chr5:3347:chr5:175224
294-175224159:1;4799/chr18:1611:chr18:76739950-76739797:2;4800/chr18:1611:chr18:76739950-76739797:1;
4801/chr15:260:chr15:28342123-28341963:2;4802/chr15:260:chr15:28342123-28341963:1;4803/chr3:2641:chr
3:138679545-138679385:2;4804/chr3:2641:chr3:138679545-138679385:1;4805/chr8:1742:chr8:55379618-5537
9458:2;4806/chr8:1742:chr8:55379618-55379458:1;4807/chr7:873:chr7:3341003-3340871:2;4808/chr7:873:chr
7:3341003-3340871:1;4809/chr11:141:chr11:849586-849411:1;4810/chr5:3128:chr5:170847735-170847579:1;4
811/chr20:54:chr20:825581-825407:2;4812/chr20:54:chr20:825581-825407:1;4813/chr4:123:chr4:996790-9966
15:2;4814/chr4:123:chr4:996790-996615:1;4815/chr14:2041:chr14:101925584-101925444:2;4816/chr14:2041:c
hr14:101925584-101925444:1;4817/chr7:735:chr7:2558718-2558588:2;4818/chr7:735:chr7:2558718-2558588:
1;4819/chr22:162:chr22:19711492-19711334:2;4820/cg11430157:chr6:118228898-118228723:2;4821/cg11430
157:chr6:118228898-118228723:1;4822/chr19:56:chr19:519152-518994:2;4823/chr19:56:chr19:519152-518994:
1;4824/chr12:438:chr12:6883150-6883011:2;4825/chr12:438:chr12:6883150-6883011:1;4826/chr11:2512:chr1
1:65553989-65553814:2;4827/chr11:2512:chr11:65553989-65553814:1;4828/chr9:2352:chr9:134430281-13443
0156:2;4829/chr9:2352:chr9:134430281-134430156:1;4830/chr10:3172:chr10:118894486-118894332:2;4831/c
hr10:3172:chr10:118894486-118894332:1;4832/chr22:162:chr22:19711492-19711334:1;4833/chr9:3497:chr9:1
39716364-139716195:1;4834/chr8:37:chr8:687199-687041:2;4835/chr8:37:chr8:687199-687041:1;4836/chr1:30
01:chr1:25255933-25255786:2;4837/chr1:3001:chr1:25255933-25255786:1;4838/chr10:4269:chr10:133110148-
133109994:2;4839/chr10:4269:chr10:133110148-133109994:1;4840/chr7:405:chr7:1276183-1276016:2;4841/c
hr7:405:chr7:1276183-1276016:1;4842/chr14:2166:chr14:103395894-103395765:2;4843/chr14:2166:chr14:103
395894-103395765:1;4844/chr19:3278:chr19:38886523-38886348:2;4845/chr19:3278:chr19:38886523-388863
48:1;4846/chr15:201:chr15:27017457-27017300:2;4847/chr15:201:chr15:27017457-27017300:1;4848/chr1:384
9:chr1:44872770-44872629:2;4849/chr1:3849:chr1:44872770-44872629:1;4850/chr15:628:chr15:41787675-417
87512:2;4851/chr15:628:chr15:41787675-41787512:1;4852/chr1:68:chr1:1007579-1007421:2;4853/chr1:68:chr
1:1007579-1007421:1;4854/chr19:2895:chr19:34113239-34113109:2;4855/chr3:2434:chr3:128764750-1287646
03:2;4856/chr3:2434:chr3:128764750-128764603:1;4857/chr2:1925:chr2:63286452-63286326:2;4858/chr2:192
5:chr2:63286452-63286326:1;4859/chr1:2566:chr1:18434805-18434631:2;4860/chr18:566:chr18:31020655-310
20493:2;4861/chr18:566:chr18:31020655-31020493:1;4862/chr8:651:chr8:10588155-10587983:2;4863/chr8:65
1:chr8:10588155-10587983:1;4864/chr1:2566:chr1:18434805-18434631:1;4865/chr15:2393:chr15:99645328-99
645153:2;4866/chr15:2393:chr15:99645328-99645153:1;4867/chr1:359:chr1:2005334-2005168:2;4868/chr1:35
9:chr1:2005334-2005168:1;4869/chr17:1937:chr17:38473237-38473062:2;4870/chr17:1937:chr17:38473237-38
473062:1;4871/chr10:2309:chr10:88123662-88123490:2;4872/chr10:2309:chr10:88123662-88123490:1;4873/c
hr19:2895:chr19:34113239-34113109:1;4874/chr4:1685:chr4:41869506-41869341:2;4875/chr4:1685:chr4:4186
9506-41869341:1;4876/chr1:1408:chr1:4714460-4714285:2;4877/chr1:1408:chr1:4714460-4714285:1;4878/chr
19:362:chr19:1457182-1457013:2;4879/chr19:362:chr19:1457182-1457013:1;4880/chr4:1277:chr4:8873162-88
72995:2;4881/chr4:1277:chr4:8873162-8872995:1;4882/chr10:801:chr10:17496439-17496267:2;4883/chr10:80
1:chr10:17496439-17496267:1;4884/chr2:3708:chr2:131721307-131721145:2;4885/chr2:3708:chr2:131721307-
131721145:1;4886/chr1:5025:chr1:119532290-119532152:2;4887/chr1:5025:chr1:119532290-119532152:1;488
8/chr2:2227:chr2:74743408-74743272:2;4889/chr13:714:chr13:53421791-53421657:2;4890/chr13:714:chr13:53
421791-53421657:1;4891/chr2:3031:chr2:111877230-111877104:2;4892/chr2:3031:chr2:111877230-11187710
4:1;4893/cg24115221:chr19:12267804-12267629:1;4894/chr13:1196:chr13:110437532-110437368:2;4895/chr1
3:1196:chr13:110437532-110437368:1;4896/chr2:118:chr2:946950-946781:2;4897/chr2:118:chr2:946950-9467
81:1;4898/chr4:2250:chr4:111542405-111542245:2;4899/chr4:2250:chr4:111542405-111542245:1;4900/chr9:1
343:chr9:110250392-110250241:2;4901/chr9:1343:chr9:110250392-110250241:1;4902/chr12:3051:chr12:1303
88203-130388062:2;4903/chr18:105:chr18:5890435-5890264:2;4904/chr6:1651:chr6:43970634-43970468:2;49
05/chr6:1651:chr6:43970634-43970468:1;4906/chr10:1637:chr10:60936875-60936701:1;4907/chr12:3051:chr1
2:130388203-130388062:1;4908/chr18:105:chr18:5890435-5890264:1;4909/chr13:917:chr13:88324765-883246
14:2;4910/chr13:917:chr13:88324765-88324614:1;4911/cg01939274:chr5:127874490-127874330:1;4912/chr20
:765:chr20:26189770-26189621:2;4913/chr20:765:chr20:26189770-26189621:1;4914/chr14:1433:chr14:931538
75-93153720:2;4915/chr14:1433:chr14:93153875-93153720:1;4916/chr4:1578:chr4:30723770-30723607:2;491
7/chr4:1578:chr4:30723770-30723607:1;4918/chr19:4944:chr19:58907723-58907574:2;4919/chr19:4944:chr19:
58907723-58907574:1;4920/chr15:867:chr15:53097635-53097469:2;4921/chr15:867:chr15:53097635-5309746
9:1;4922/chr19:2031:chr19:17439446-17439277:1;4923/chr5:3644:chr5:178003946-178003802:2;4924/chr5:36
44:chr5:178003946-178003802:1;4925/chr13:1702:chr13:113764769-113764604:2;4926/chr13:1702:chr13:113
764769-113764604:1;4927/chr2:2227:chr2:74743408-74743272:1;4928/chr7:1568:chr7:27260422-27260255:2;
4929/chr7:1568:chr7:27260422-27260255:1;4930/chr4:774:chr4:5894776-5894619:2;4931/chr4:774:chr4:58947
76-5894619:1;4932/chr3:2134:chr3:123167101-123166936:2;4933/chr4:1578:chr4:30723902-30723731:2;4934/
chr4:1578:chr4:30723902-30723731:1;4935/chr3:2134:chr3:123167101-123166936:1;4936/chr2:4772:chr2:220
314052-220313898:2;4937/chr9:1073:chr9:96715514-96715350:2;4938/chr9:1073:chr9:96715514-96715350:1;
4939/cg09231862:chr14:29235470-29235306:1;4940/chr2:4772:chr2:220314052-220313898:1;4941/chr18:608:
chr18:34824030-34823862:2;4942/chr12:1090:chr12:50355023-50354892:2;4943/chr12:1090:chr12:50355023-
50354892:1;4944/chr18:608:chr18:34824030-34823862:1;4945/chr12:2890:chr12:127211067-127210910:2;494
6/chr12:2890:chr12:127211067-127210910:1;4947/chr2:1706:chr2:4759697547596826:2;4948/chr2:1706:chr2:
47596975-47596826:1;4949/chrX:1339:chrX:108868486-108868311:2;4950/chrX:1339:chrX:108868486-10886
8311:1;4951/chr11:4678:chr11:130029861-130029713:2;4952/chr11:4678:chr11:130029861-130029713:1;4953/
chr13:368:chr13:28674541-28674389:2;4954/chr13:368:chr13:28674541-28674389:1;4955/chr3:2544:chr3:134
031997-134031826:2;4956/chr3:2544:chr3:134031997-134031826:1;4957/chr1:224:chr1:1475773-1475598:2;4
958/chr1:224:chr1:1475773-1475598:1;4959/chr20:2210:chr20:59828197-59828051:2;4960/chr20:2210:chr20:5
9828197-59828051:1;4961/chr17:1098:chr17:13504849-13504674:2;4962/chr17:1098:chr17:13504849-135046
74:1;4963/chr6:1147:chr6:33245407-33245262:2;4964/chr6:1147:chr6:33245407-33245262:1;4965/chr16:234:c
hr16:1035168-1035008:2;4966/chr16:234:chr16:1035168-1035008:1;4967/chr9:1081:chr9:96721611-96721438:
2;4968/chr9:1081:chr9:96721611-96721438:1;4969/chr19:2725:chr19:31839668-31839505:2;4970/chr19:2725:
chr19:31839668-31839505:1;4971/chr3:2770:chr3:147128533-147128358:2;4972/chr3:2770:chr3:147128533-1
47128358:1;4973/chr20:2210:chr20:59827213-59827085:2;4974/chr20:2210:chr20:59827213-59827085:1;4975/
chr6:99:chr6:1384388-1384228:2;4976/chr6:99:chr6:1384388-1384228:1;4977/chr14:454:chr14:48144340-481
44169:2;4978/chr14:454:chr14:48144340-48144169:1;4979/chr17:2301:chr17:45500937-45500762:2;4980/chr1
7:2301:chr17:45500937-45500762:1;4981/chr2:2852:chr2:107503158-107502987:2;4982/chr2:2852:chr2:10750
3158-107502987:1;4983/chr19:1946:chr19:16436536-16436381:2;4984/chr19:1946:chr19:16436536-16436381:
1;4985/chr8:1309:chr8:35093114-35092970:2;4986/chr8:1309:chr8:35093114-35092970:1;4987/chr11:98:chr11:
627499-627331:2;4988/chr11:98:chr11:627499-627331:1;4989/chr22:2210:chr22:48972735-48972562:2;4990/
chr7:873:chr7:3341078-3340952:2;4991/chr7:873:chr7:3341078-3340952:1;4992/chr22:2210:chr22:48972735-4
8972562:1;4993/chr12:1460:chr12:58130245-58130113:1;4994/chr7:1536:chr7:27205963-27205792:2;4995/chr
17:2190:chr17:43198610-43198469:2;4996/chr17:2190:chr17:43198610-43198469:1;4997/chr8:1716:chr8:5478
9885-54789710:2;4998/chr8:1716:chr8:54789885-54789710:1;4999/chr7:1536:chr7:27205963-27205792:1;500
0/chr17:1218:chr17:17399510-17399341:2;5001/chr17:1218:chr17:17399510-17399341:1;5002/chr6:380:chr6:1
0382217-10382080:2;5003/chr12:2984:chr12:129338259-129338114:2;5004/chr12:2984:chr12:129338259-129
338114:1;5005/chr6:380:chr6:10382217-10382080:1;5006/chr17:276:chr17:1961174-1961001:2;5007/chr17:27
6:chr17:1961174-1961001:1;5008/chr1:5019:chr1:119527190-119527022:1;5009/chr2:4191:chr2:175205431-17
5205257:2;5010/chr2:4191:chr2:175205431-175205257:1;5011/chr4:1727:chr4:44450459-44450285:2;5012/chr
15:207:chr15:27112514-27112369:2;5013/chr15:207:chr15:27112514-27112369:1;5014/chr5:3993:chr5:180486
476-180486301:2;5015/chr5:3993:chr5:180486476-180486301:1;5016/chr4:1727:chr4:4445045944450285:1;5
017/chr22:2676:chr22:50496898-50496727:2;5018/chr22:2676:chr22:50496898-50496727:1;5019/chr16:74:chr
16:610719-610546:2;5020/chr16:74:chr16:610719-610546:1;5021/chr6:107:chr6:1393843-1393708:2;5022/chr6:
107:chr6:1393843-1393708:1;5023/chr9:1878:chr9:129377608-129377444:2;5024/chr9:1878:chr9:129377608-
129377444:1;5025/chr3:3168:chr3:184301702-184301573:2;5026/chr3:3168:chr3:184301702-184301573:1;502
7/chr8:2717:chr8:139509239-139509073:2;5028/chr8:2717:chr8:139509239-139509073:1;5029/chrX:1761:chrX:
151143025-151142878:2;5030/chrX:1761:chrX:151143025-151142878:1;5031/chr12:3094:chr12:130646795-1
30646628:2;5032/chr12:3094:chr12:130646795-130646628:1;5033/chr16:3585:chr16:85320785-85320612:2;50
34/chr16:3585:chr16:85320785-85320612:1;5035/chr13:289:chr13:26626263-26626088:1;5036/chr10:2761:chr
10:102987065-102986897:2;5037/chr10:2761:chr10:102987065-102986897:1;5038/chr6:2396:chr6:117869487-
117869358:1;5039/chr12:3097:chr12:130648115-130647973:2;5040/chr12:3097:chr12:130648115-130647973:
1;5041/chr5:705:chr5:2751322-2751197:2;5042/chr5:705:chr5:2751322-2751197:1;5043/chr9:3136:chr9:13860
6462-138606291:2;5044/chr9:3136:chr9:138606462-138606291:1;5045/chr6:1567:chr6:42072269-42072127:2;
5046/chr6:1567:chr6:42072269-42072127:1;5047/chr19:368:chr19:1469993-1469823:2;5048/chr19:368:chr19:1
469993-1469823:1;5049/chr14:2038:chr14:101923723-101923592:2;5050/chr14:2038:chr14:101923723-10192
3592:1;5051/chr5:1725:chr5:72732733-72732563:2;5052/chr5:1725:chr5:72732733-72732563:1;5053/chr5:204
8:chr5:112539175-112539000:1;5054/chr7:1422:chr7:25892704-25892570:1;5055/chr6:384:chr6:10385214-103
85039:1;5056/chr16:825:chr16:3550241-3550067:1;5057/chr1:3856:chr1:44883530-44883355:2;5058/chr1:385
6:chr1:44883530-44883355:1;5059/chr1:3953:chr1:47899983-47899857:2;5060/chr1:3953:chr1:47899983-4789
9857:1;5061/chr7:1908:chr7:39649358-39649183:2;5062/chr4:1288:chr4:8895032-8894859:1;5063/chr14:397:c
hr14:38091702-38091567:2;5064/chr14:397:chr14:38091702-38091567:1;5065/chr2:4784:chr2:220361610-220
361435:2;5066/chr2:4784:chr2:220361610-220361435:1;5067/chr2:4245:chr2:176981383-176981210:2;5068/c
hr2:4245:chr2:176981383-176981210:1;5069/chr18:280:chr18:11751833-11751658:2;5070/chr19:4653:chr19:5
5598405-55598269:2:5071/chr19:4653:chr19:55598405-55598269:1;5072/chr22:15:chr22:17083238-17083064:
2;5073/chr22:15:chr22:17083238-17083064:1;5074/chr10:3406:chr10:123923438-123923267:2;5075/chr10:340
6:chr10:123923438-123923267:1;5076/chr15:634:chr15:41795310-41795135:2;5077/chr15:634:chr15:4179531
0-41795135:1;5078/chr12:1321:chr12:54428617-54428443:2;5079/chr12:1321:chr12:54428617-54428443:1;50
80/chr13:45:chr13:20176198-20176064:2;5081/chr13:45:chr13:20176198-20176064:1;5082/chr18:280:chr18:11
751833-11751658:1;5083/chr4:207:chr4:1400658-1400488:2;5084/chr4:207:chr4:1400658-1400488:1;5085/chr
2:3447:chr2:127414109-127413935:1;5086/chr15:935:chr15:58357378-58357203:1;5087/chr7:4539:chr7:15558
0257-155580082:2;5088/chr7:4539:chr7:155580257-155580082:1;5089/chr5:1789:chr5:76373779-76373648:2;
5090/chr5:1789:chr5:76373779-76373648:1;5091/chr10:2140:chr10:81002973-81002834:2;5092/chr10:2140:ch
r10:81002973-81002834:1;5093/chr7:2162:chr7:50343573-50343428:2;5094/chr7:2162:chr7:50343573-503434
28:1;5095/chr7:1908:chr7:39649358-39649183:1;5096/chr8:766:chr8:12809098-12808967:2;5097/chr8:766:chr
8:12809098-12808967:1;5098/chr6:21:chr6:392493-392340:2;5099/chr6:21:chr6:392493-392340:1;5100/chr17:
1003:chr17:11144167-11144024:1;5101/chr16:4109:chr16:88497153-88497023:2;5102/chr16:4109:chr16:8849
7153-88497023:1;5103/chr19:3497:chr19:41106268-41106143:2;5104/chr19:3497:chr19:41106268-41106143:1;
5105/chr8:791:chr8:15397882-15397749:2;5106/chr8:791:chr8:15397882-15397749:1;5107/chr19:1696:chr19:
13210368-13210193:2;5108/chr19:1696:chr19:13210368-13210193:1;5109/chr10:3406:chr10:123923024-1239
22851:1;5110/chr12:2890:chr12:127211222-127211048:2;5111/chr12:2890:chr12:127211222-127211048:1;511
2/chr17:550:chr17:4981877-4981752:2;5113/chr17:550:chr17:4981877-4981752:1;5114/chr8:3759:chr8:145698
233-145698086:2;5115/chr8:3759:chr8:145698233-145698086:1;5116/chr2:3107:chr2:115919305-115919132:2;
5117/chr2:3107:chr2:115919305-115919132:1;5118/chr7:4817:chr7:157478108-157477978:2;5119/chr7:4817:
chr7:157478108-157477978:1;5120/chr6:458:chr6:12750116-12749942:2;5121/chr6:458:chr6:12750116-12749
942:1;5122/chr20:516:chr20:21082095-21081964:2;5123/chr20:516:chr20:21082095-21081964:1;5124/chr17:2
750:chr17:56565024-56564849:2:5125/chr17:2750:chr17:56565024-56564849:1;5126/chr4:2084:chr4:8541914
5-85419019:2;5127/chr4:2084:chr4:85419145-85419019:1;5128/chr19:4173:chr19:50393886-50393711:2;5129/
chr6:2842:chr6:157557665-157557498:2;5130/chr6:2842:chr6:157557665-157557498:1;5131/chr4:637:chr4:37
69422-3769249:2;5132/chr4:637:chr4:3769422-3769249:1;5133/chr13:699:chr13:53174626-53174459:2;5134/c
hr13:699:chr13:53174626-53174459:1;5135/chr17:4140:chr17:77789784-77789613:2:5136/chr17:4140:chr17:7
7789784-77789613:1;5137/chr13:341:chr13:28498448-28498275:2;5138/chr17:4130:chr17:77776730-7777655
5:2;5139/chr17:4130:chr17:77776730-77776555:1;5140/chr5:1949:chr5:92940104-92939930:2;5141/chr5:1949:
chr5:92940104-92939930:1;5142/chr14:1947:chr14:101193429-101193256:2;5143/chr5:506:chr5:1882419-188
2244:1;5144/chr6:1534:chr6:4151610541515930:2;5145/chr6:1534:chr6:4151610541515930:1;5146/chr22:18
52:chr22:46476100-46475974:2;5147/chr22:1852:chr22:46476100-46475974:1;5148/chr3:1154:chr3:48693870-
48693701:2;5149/chr3:1154:chr3:48693870-48693701:1;5150/chr1:3849:chr1:44872886-44872716:1;5151/chr2:
117:chr2:946294-946125:1;5152/chr10:4449:chr10:133999386-133999214:1;5153/chr16:4191:chr16:88701006-
88700876:2:5154/chr16:4191:chr16:88701006-88700876:1;5155/chr7:1497:chr7:27155388-27155256:2;5156/c
hr7:1497:chr7:27155388-27155256:1;5157/chr5:705:chr5:2751267-2751092:2;5158/chr5:705:chr5:2751267-27
51092:1;5159/chr20:2983:chr20:62369570-62369427:1;5160/chr10:3406:chr10:123923081-123922956:2:5161/
chr10:3406:chr10:123923081-123922956:1;5162/chr14:312:chr14:36973320-36973145:2;5163/chr14:312:chr14:
36973320-36973145:1;5164/chr7:4691:chr7:156796818-156796643:2:5165/chr7:4691:chr7:156796818-156796
643:1;5166/chr19:1881:chr19:15580732-15580557:2;5167/chr19:1881:chr19:15580732-15580557:1;5168/chr10:
3171:chr10:118893967-118893793:2;5169/chr10:3171:chr10:118893967-118893793:1:5170/chr3:431:chr3:135
90906-13590731:2;5171/chr3:431:chr3:13590906-13590731:1;5172/chr7:2589:chr7:70597337-70597185:2;517
3/chr7:2589:chr7:70597337-70597185:1:5174/chr6:86:chr6:1312516-1312343:2;5175/chr6:86:chr6:1312516-13
12343:1;5176/chr6:2842:chr6:157557913-157557740:2;5177/chr6:2842:chr6:157557913-157557740:1;5178/chr
19:4173:chr19:50393886-50393711:1;5179/chr13:917:chr13:88325013-88324882:2;5180/chr13:917:chr13:8832
5013-88324882:1;5181/chr9:1226:chr9:100616551-100616376:2;5182/chr9:1226:chr9:100616551-100616376:1;
5183/chr13:341:chr13:28498448-28498275:1;5184/chr5:705:chr5:2751744-2751576:2;5185/chr5:705:chr5:275
1744-2751576:1;5186/chr22:2727:chr22:50738088-50737947:2;5187/chr22:2727:chr22:50738088-50737947:1;
5188/chr1:1852:chr1:8277618-8277493:2;5189/chr1:1852:chr1:8277618-8277493:1;5190/chr10:906:chr10:2348
1221-23481092:2;5191/chr10:906:chr10:23481221-23481092:1;5192/chr2:5862:chr2:242498304-242498177:2;
5193/chr2:5862:chr2:242498304-242498177:1;5194/chr14:1947:chr14:101193429-101193256:1;5195/chr11:10
79:chr11:6651602-6651428:2;5196/chr11:1079:chr11:6651602-6651428:1;5197/chr12:1321:chr12:54428791-54
428617:2;5198/chr12:1321:chr12:54428791-54428617:1;5199/chrX:1560:chrX:133684982-133684808:1;5200/c
hr1:3851:chr1:44873255-44873082:2;5201/chr1:3851:chr1:44873255-44873082:1;5202/chr2:4214:chr2:176944
944-176944769:2;5203/chr2:4214:chr2:176944944-176944769:1;5204/chr2:1902:chr2:63275143-63275012:2;5
205/chr2:1902:chr2:63275143-63275012:1;5206/chr18:105:chr18:5890930-5890796:2;5207/chr18:105:chr18:58
90930-5890796:1;5208/chr5:3119:chr5:170737324-170737149:2;5209/chr17:1098:chr17:13504921-13504746:2;
5210/chr17:1098:chr17:13504921-13504746:1;5211/chr2:1181:chr2:24233170-24233045:2;5212/chr2:1181:chr
2:24233170-24233045:1;5213/chr12:1319:chr12:54427781-54427637:1;5214/chr17:1814:chr17:35300356-3530
0222:2;5215/chr17:1814:chr17:35300356-35300222:1;5216/chr5:3119:chr5:170737324-170737149:1;5217/chr1
2:1084:chr12:50297660-50297510:1;5218/chr7:3112:chr7:100091322-100091176:2;5219/chr7:547:chr7:157702
9-1576903:2;5220/chr7:547:chr7:1577029-1576903:1;5221/chr19:2032:chr19:17439780-17439654:1;5222/chr7:
3112:chr7:100091322-100091176:1;5223/chr22:1248:chr22:40058309-40058177:2;5224/chr22:1248:chr22:400
58309-40058177:1;5225/chr18:891:chr18:47795096-47794921:1;5226/chr15:1477:chr15:75248867-75248694:2;
5227/chr15:1477:chr15:75248867-75248694:1;5228/chr12:3052:chr12:130389012-130388837:1;5229/chr1:395
7:chr1:47905373-47905230:2;5230/chr1:3957:chr1:47905373-47905230:1;5231/chr19:553:chr19:2252169-2251
996:2;5232/chr19:553:chr19:2252169-2251996:1;5233/chr3:160:chr3:9595467-9595324:2;5234/chr3:160:chr3:9
595467-9595324:1;5235/chr19:2031:chr19:17439119-17438991:2;5236/chr19:2031:chr19:17439119-17438991:
1;5237/chr9:47:chr9:976769-976594:2;5238/chr9:47:chr9:976769-976594:1;5239/chr13:452:chr13:31480794-31
480644:2;5240/chr13:452:chr13:31480794-31480644:1;5241/chr2:3108:chr2:115919599-115919444:2;5242/chr
2:3108:chr2:115919599-115919444:1;5243/chr1:7412:chr1:248021052-248020917:2;5244/chr1:7412:chr1:2480
21052-248020917:1;5245/chr8:1720:chr8:54792258-54792133:1;5246/chr2:4217:chr2:176947321-176947190:1;
5247/chr3:3063:chr3:179755035-179754866:2;5248/chr3:3063:chr3:179755035-179754866:1;5249/chr3:2382:
chr3:128210984-128210811:2;5250/chr3:2382:chr3:128210984-128210811:1;5251/chr7:3501:chr7:127671733-
127671605:2;5252/chr7:3501:chr7:127671733-127671605:1;5253/chr15:937:chr15:58358020-58357871:2;5254/
chr15:937:chr15:58358020-58357871:1;5255/chr14:1032:chr14:75078378-75078215:2;5256/chr14:1032:chr14:
75078378-75078215:1;5257/chr1:645:chr1:2706747-2706579:2;5258/chr1:645:chr1:2706747-2706579:1;5259/c
hr14:1600:chr14:95826920-95826747:2;5260/chr14:1600:chr14:95826920-95826747:1;5261/chr19:3999:chr19:
48946952-48946809:2;5262/chr19:3999:chr19:48946952-48946809:1;5263/chr2:3075:chr2:114035278-114035
108:2;5264/chr2:3075:chr2:114035278-114035108:1;5265/chr4:1487:chr4:21950629-21950473:2;5266/chr4:14
87:chr4:21950629-21950473:1;5267/chr14:986:chr14:73958477-73958311:2;5268/chr14:986:chr14:73958477-7
3958311:1;5269/chr1:3851:chr1:44873540-44873376:2;5270/chr1:3851:chr1:44873540-44873376:1;5271/chr3:
2383:chr3:128211406-128211240:2;5272/chr3:2383:chr3:128211406-128211240:1;5273/chr17:3502:chr17:737
49852-73749685:2;5274/chr17:3502:chr17:73749852-73749685:1;5275/chr22:14:chr22:17082595-17082432:2;
5276/chr22:14:chr22:17082595-17082432:1;5277/chr21:666:chr21:44037292-44037121:1;5278/chr5:2324:chr5:
134363570-134363445:2;5279/chr5:2324:chr5:134363570-134363445:1;5280/chr9:3497:chr9:139716121-1397
15964:2;5281/chr9:3497:chr9:139716121-139715964:1;5282/chr5:504:chr5:1878738-1878612:2;5283/chr5:504:
chr5:1878738-1878612:1;5284/chr1:7249:chr1:240256188-240256063:2;5285/chr1:7249:chr1:240256188-2402
56063:1;5286/chrX:678:chrX:39949450-39949303:2;5287/chrX:678:chrX:39949450-39949303:1;5288/chr9:948:
chr9:94712148-94711994:2;5289/chr5:3837:chr5:178957607-178957432:2;5290/chr5:3837:chr5:178957607-17
8957432:1;5291/chr20:946:chr20:32857247-32857102:1;5292/chr9:948:chr9:94712148-94711994:1;5293/chr8:
1720:chr8:54792139-54791967:2;5294/chr8:1720:chr8:54792139-54791967:1;5295/chr10:4270:chr10:1331105
09-133110378:2;5296/chr10:4270:chr10:133110509-133110378:1;5297/chr14:2219:chr14:103739908-1037397
47:2;5298/chr14:2219:chr14:103739908-103739747:1;5299/chr11:3411:chr11:74952774-74952632:2;5300/chr1
1:3411:chr1174952774-74952632:1;5301/chr11:4527:chr11:126286483-126286358:2;5302/chr11:4527:chr11:1
26286483-126286358:1;5303/chr22:1852:chr22:46476252-46476094:2;5304/chr22:1852:chr22:46476252-4647
6094:1;5305/chrX:27:chrX:334621-334496:2;5306/chrX:27:chrX:334621-334496:1;5307/chrX:693:chrX:400051
32-40004973:2;5308/chrX:693:chrX:40005132-40004973:1;5309/chr4:2716:chr4:174430804-174430631:2;5310/
chr4:2716:chr4:174430804-174430631:1;5311/chr18:91:chr18:5543341-5543178:2;5312/chr18:91:chr18:55433
41-5543178:1;5313/chr5:504:chr5:1878925-1878799:2;5314/chr5:504:chr5:1878925-1878799:1;5315/chr8:1878:
chr8:65282803-65282628:2;5316/chr8:594:chr8:9764211-9764047:2;5317/chr8:594:chr8:9764211-9764047:1;5
318/chr8:1878:chr8:65282803-65282628:1;5319/chr20:524:chr20:21376757-21376620:2;5320/chr20:524:chr20:
21376757-21376620:1;5321/chr22:1823:chr22:46368222-46368053:2;5322/chr22:1823:chr22:46368222-46368
053:1;5323/chr13:699:chr13:53174743-53174614:1;5324/chr5:3113:chr5:170289224-170289073:2;5325/chr5:3
113:chr5:170289224-170289073:1;5326/chr9:1073:chr9:96715573-96715419:2;5327/chr9:1073:chr9:96715573-
96715419:1;5328/chr19:1987:chr19:17008857-17008683:2;5329/chr19:1987:chr19:17008857-17008683:1;5330/
chr13:1076:chr13:100641563-100641438:1;5331/chr4:1481:chr4:20256796-20256625:2;5332/chr4:1481:chr4:2
0256796-20256625:1;5333/chr20:134:chr20:2673116-2672991:2;5334/chr20:134:chr20:2673116-2672991:1;53
35/chr5:3298:chr5:174159022-174158853:2;5336/chr5:3298:chr5:174159022-174158853:1;5337/chr16:110:chr
16:744579-744436:2;5338/chr16:110:chr16:744579-744436:1;5339/chr12:1523:chr12:65219000-65218840:2;53
40/chr12:1523:chr12:65219000-65218840:1;5341/chr2:4215:chr2:176945653-176945526:2;5342/chr2:4215:chr
2:176945653-176945526:1;5343/chr17:1003:chr17:11144841-11144714:2;5344/chr17:1003:chr17:11144841-11
144714:1;5345/chr5:2324:chr5:134363847-134363677:2;5346/chr5:2324:chr5:134363847-134363677:1;5347/c
hr19:3503:chr19:41119623-41119462:2;5348/chr19:3503:chr19:41119623-41119462:1;5349/chr5:1718:chr5:72
677608-72677479:2;5350/chr5:1718:chr5:72677608-72677479:1;5351/chr8:3759:chr8:145698410-145698258:2;
5352/chr8:3759:chr8:145698410-145698258:1;5353/chr19:4944:chr19:58908421-58908285:2;5354/chr19:4944:
chr19:58908421-58908285:1;5355/chr16:756:chr16:3017179-3017027:2;5356/chr9:2443:chr9:135462360-1354
62199:2;5357/chr9:2443:chr9:135462360-135462199:1;5358/chr13:874:chr13:79177791-79177622:2;5359/chr1
3:874:chr13:79177791-79177622:1;5360/chr9:1715:chr9:126780359-126780185:2;5361/chr9:1715:chr9:126780
359-126780185:1;5362/chr7:3412:chr7:113727785-113727612:2;5363/chr7:3412:chr7:113727785-113727612:1;
5364/chr9:1682:chr9:126135674-126135511:2;5365/chr9:1682:chr9:126135674-126135511:1;5366/chr1:7249:
chr1:240256616-240256443:2;5367/chr10:861:chr10:22542464-22542294:2;5368/chr10:861:chr10:22542464-2
2542294:1;5369/chr1:7249:chr1:240256616-240256443:1;5370/chr15:2252:chr15:95388255-95388088:2;5371/
chr15:2252:chr15:95388255-95388088:1;5372/chr18:610:chr18:34833717-34833542:2;5373/chr18:610:chr18:3
4833717-34833542:1;5374/chr2:4252:chr2:176987935-176987797:2;5375/chr2:4252:chr2:176987935-1769877
97:1;5376/chr19:830:chr19:3786494-3786349:2;5377/chr19:830:chr19:3786494-3786349:1;5378/chr11:2301:ch
r11:63768200-63768040:2;5379/chr11:2301:chr11:63768200-63768040:1;5380/chr2:597:chr2:5832344-583219
7:2;5381/chr2:597:chr2:5832344-5832197:1;5382/chr13:1471:chr13:112758896-112758729:2;5383/chr13:1471:
chr13:112758896-112758729:1;5384/chr16:756:chr16:3017179-3017027:1;5385/chr4:5:chr4:107740-107572:2;
5386/chr4:5:chr4:107740-107572:1;5387/chr14:164:chr14:24804200-24804029:2;5388/chr14:164:chr14:248042
00-24804029:1;5389/chr11:2667:chr11:67290900-67290774:2;5390/chr10:2140:chr10:81003591-81003466:2;5
391/chr10:2140:chr10:81003591-81003466:1;5392/chr11:2667:chr11:67290900-67290774:1;5393/chr8:2717:ch
r8:139509372-139509235:2;5394/chr8:2717:chr8:139509372-139509235:1;5395/chr6:279:chr6:6004190-60040
64:2;5396/chr6:279:chr6:6004190-6004064:1;5397/chr3:1010:chr3:45187643-45187472:2;5398/chr3:1010:chr3:
45187643-45187472:1;5399/chr13:1456:chr13:112721373-112721238:2;5400/chr13:1456:chr13:112721373-11
2721238:1;5401/chr3:649:chr3:22414339-22414212:2;5402/chr3:649:chr3:22414339-22414212:1;5403/chr16:3
821:chr16:86542194-86542027:2;5404/chr16:3821:chr16:86542194-86542027:1;5405/chr11:2279:chr11:63530
682-63530552:2;5406/chr11:2279:chr11:63530682-63530552:1;5407/chr1:3435:chr1:34642803-34642634:2;54
08/chr8:1309:chr8:35092835-35092697:2;5409/chr8:1309:chr8:35092835-35092697:1;5410/chr1:3435:chr1:346
42803-34642634:1;5411/chr10:583:chr10:11059819-11059656:2;5412/chr10:583:chr10:11059819-11059656:1;
5413/chr2:4214:chr2:176945230-176945060:2;5414/chr2:4214:chr2:176945230-176945060:1;5415/chr3:2518:c
hr3:133393345-133393194:2;5416/chr10:629:chr10:11784863-11784721:2;5417/chr10:629:chr10:11784863-11
784721:1;5418/chr3:2518:chr3:133393345-133393194:1;5419/chr7:727:chr7:2539938-2539765:2;5420/chr7:72
7:chr7:2539938-2539765:1;5421/chr11:716:chr11:2291738-2291563:2;5422/chr11:716:chr11:2291738-2291563:
1;5423/chr17:4657:chr17:80332974-80332803:2;5424/chr17:4657:chr17:80332974-80332803:1;5425/chr7:415
3:chr7:150811879-150811754:2;5426/chr7:4153:chr7:150811879-150811754:1;5427/chr6:1938:chr6:79620476-
79620301:2;5428/chr6:1938:chr6:79620476-79620301:1;5429/chr19:367:chr19:1467728-1467567:2;5430/chr19:
367:chr19:1467728-1467567:1;5431/chr2:1230:chr2:25500461-25500292:2;5432/chr2:1230:chr2:25500461-25
500292:1;5433/chr12:1463:chr12:58132114-58131969:2;5434/chr12:1463:chr12:58132114-58131969:1;5435/c
hr22:2205:chr22:4897112848970969:2;5436/chr22:2205:chr22:48971128-48970969:1;5437/chr4:2647:chr4:16
4265281-164265134:2;5438/chr4:2647:chr4:164265281-164265134:1;5439/chr13:1196:chr13:110437296-1104
37128:2;5440/chr13:1196:chr13:110437296-110437128:1;5441/chr2:4772:chr2:220313440-220313299:2;5442/
chr2:4772:chr2:220313440-220313299:1;5443/chr4:774:chr4:5894672-5894541:2;5444/chr4:774:chr4:5894672-
5894541:1;5445/chr22:2210:chr22:48972853-48972678:2;5446/chr8:1408:chr8:38965081-38964934:1;5447/chr
8:3701:chr8:145104091-145103917:2;5448/chr8:3701:chr8:145104091-145103917:1;5449/chr6:2232:chr6:1079
56061-107955917:2;5450/chr6:2232:chr6:107956061-107955917:1;5451/chr7:3500:chr7:127671355-12767122
9:2;5452/chr7:3500:chr7:127671355-127671229:1;5453/chr13:657:chr13:49795205-49795039:2;5454/chr1:431
3:chr1:60280719-60280554:2;5455/chr1:4313:chr1:60280719-60280554:1;5456/chr3:2471:chr3:129693700-129
693568:2;5457/chr7:4146:chr7:150778613-150778446:2;5458/chr7:4146:chr7:150778613-150778446:1;5459/c
hr16:2915:chr16:67918835-67918666:2;5460/chr22:1865:chr22:46484808-46484636:2;5461/chr22:1865:chr22:
46484808-46484636:1;5462/chr4:1727:chr4:44450517-44450343:2;5463/chr11:1214:chr11:13032129-1303196
7:1;5464/chr12:1523:chr12:65218784-65218610:2;5465/chr12:1523:chr12:65218784-65218610:1;5466/chr4:15
78:chr4:30723658-30723532:2;5467/chr4:1578:chr4:30723658-30723532:1;5468/chr15:1553:chr15:76635273-7
6635098:2;5469/chr15:1553:chr15:76635273-76635098:1;5470/chr8:791:chr8:15398047-15397872:2;5471/chr8:
791:chr8:15398047-15397872:1;5472/chr4:1727:chr4:44450517-44450343:1;5473/chr6:256:chr6:5086441-508
6294:2;5474/chr6:256:chr6:5086441-5086294:1;5475/chr9:1070:chr9:96714322-96714197:2;5476/chr9:1070:ch
r9:96714322-96714197:1;5477/chr2:4784:chr2:220361507-220361382:2;5478/chr2:4784:chr2:220361507-2203
61382:1;5479/chr12:156:chr12:2800605-2800437:2;5480/chr12:156:chr12:2800605-2800437:1;5481/chr18:163
3:chr18:76829012-76828885:2;5482/chr18:1633:chr18:76829012-76828885:1;5483/chr1:7249:chr1:240256486-
240256337:2;5484/chr1:7249:chr1:240256486-240256337:1;5485/chr3:2471:chr3:129693700-129693568:1;548
6/chr16:2915:chr16:67918835-67918666:1;5487/chr9:3508:chr9:139740919-139740779:2;5488/chr9:3508:chr9:
139740919-139740779:1;5489/chr5:2252:chr5:132150172-132150039:2;5490/chr5:2252:chr5:132150172-1321
50039:1;5491/chr10:2140:chr10:81003378-81003209:2;5492/chr10:2140:chr10:81003378-81003209:1;5493/chr
6:95:chr6:1378528-1378401:2;5494/chr6:95:chr6:1378528-1378401:1;5495/chr4:123:chr4:996842-996711:2;54
96/chr4:123:chr4:996842-996711:1;5497/chr4:2084:chr4:85418842-85418683:2;5498/chr4:2084:chr4:85418842-
85418683:1;5499/chr17:3502:chr17:73750135-73749961:2;5500/chr6:1567:chr6:42072583-42072412:2;5501/c
hr6:1567:chr6:42072583-42072412:1;5502/chr2:1230:chr2:25500083-25499935:1;5503/chr17:3502:chr17:7375
0135-73749961:1;5504/chr22:1894:chr22:46659087-46658953:2;5505/chr22:1894:chr22:46659087-46658953:1;
5506/chr19:372:chr19:1471096-1470952:2;5507/chr19:372:chr19:1471096-1470952:1;5508/chr19:2257:chr19:
19335978-19335806:2;5509/chr19:2257:chr19:19335978-19335806:1;5510/chr20:136:chr20:2674487-2674323:
2;5511/chr20:136:chr20:2674487-2674323:1;5512/chr5:3993:chr5:180486615-180486467:2;5513/chr10:1587:c
hr10:52178053-52177922:2;5514/chr10:1587:chr10:52178053-52177922:1;5515/chr5:3993:chr5:180486615-18
0486467:1;5516/chr22:2210:chr22:48972509-48972381:2;5517/chr22:2210:chr22:48972509-48972381:1;5518/
chr12:1321:chr12:54428459-54428298:2;5519/chr12:1321:chr12:54428459-54428298:1;5520/chr22:2210:chr22:
48972853-48972678:1;5521/chr9:948:chr9:94712231-94712062:2;5522/chr9:948:chr9:94712231-94712062:1;5
523/chr15:982:chr15:60296746-60296613:2;5524/chr15:982:chr15:60296746-60296613:1;5525/chr13:657:chr1
3:49795205-49795039:1;5526/chr3:393:chr3:13324475-13324326:2;5527/chr3:393:chr3:13324475-13324326:1;
5528/chr11:3411:chr11:74952901-74952729:2;5529/chr11:3411:chr11:74952901-74952729:1;5530/chr15:753:c
hr15:45479850-45479695:2;5531/chr15:753:chr15:45479850-45479695:1;5532/chr17:2514:chr17:48194870-48
194744:2;5533/chr17:2514:chr17:48194870-48194744:1;5534/chr2:3916:chr2:154335137-154334996:2;5535/c
hr2:3916:chr2:154335137-154334996:1;5536/chr16:3825:chr16:86544916-86544742:2;5537/chr16:3825:chr16:
86544916-86544742:1;5538/chr3:2641:chr3:138679318-138679193:2;5539/chr3:2641:chr3:138679318-138679
193:1;5540/chr1:68:chr1:1007483-1007340:2;5541/chr1:68:chr1:1007483-1007340:1;5542/chr19:551:chr19:225
1545-2251370:2;5543/chr19:551:chr19:2251545-2251370:1;5544/chr11:2420:chr11:64428464-64428291:2;554
5/chr11:2420:chr11:64428464-64428291:1;5546/chr2:3916:chr2:154335020-154334845:2;5547/chr2:3916:chr2:
154335020-154334845:1;5548/chr8:809:chr8:17271061-17270911:2;5549/chr8:809:chr8:17271061-17270911:1;
5550/chr19:1696:chr19:13209854-13209703:2;5551/chr19:1696:chr19:13209854-13209703:1;5552/chr7:1491:
chr7:27153235-27153110:2;5553/chr7:1491:chr7:27153235-27153110:1;5554/chr15:2252:chr15:95388328-953
88168:2;5555/chr15:2252:chr15:95388328-95388168:1;5556/chr7:4153:chr7:150812676-150812548:2;5557/chr
7:4153:chr7:150812676-150812548:1;5558/chr2:741:chr2:8816991-8816821:2;5559/chr2:741:chr2:8816991-88
16821:1;5560/chr6:920:chr6:30039293-30039121:2;5561/chr6:920:chr6:30039293-30039121:1;5562/chr10:270
0:chr10:102587763-102587599:2;5563/chr10:2700:chr10:102587763-102587599:1;5564/chr6:279:chr6:600395
3-6003785:2;5565/chr6:279:chr6:6003953-6003785:1;5566/chr2:2749:chr2:105459466-105459295:2;5567/chr2:
2749:chr2:105459466-105459295:1;5568/chr19:298:chr19:1257140-1256974:2;5569/chr19:298:chr19:1257140-
1256974:1;5570/chr14:2041:chr14:101925470-101925345:2;5571/chr14:2041:chr14:101925470-101925345:1;5
572/chr9:2223:chr9:133540306-133540144:2;5573/chr9:2223:chr9:133540306-133540144:1;5574/chr17:270:ch
r17:1958979-1958835:2;5575/chr17:270:chr17:1958979-1958835:1;5576/chr3:3077:chr3:181413485-18141336
0:2;5577/chr3:3077:chr3:181413485-181413360:1;5578/chr7:1533:chr7:27205022-27204847:2;5579/chr7:1533:
chr7:27205022-27204847:1;5580/chr2:4262:chr2:176995118-176994971:2;5581/chr2:4262:chr2:176995118-17
6994971:1;5582/chr1:604:chr1:2461877-2461732:2;5583/chr1:604:chr1:2461877-2461732:1;5584/chr17:2173:c
hr17:43037477-43037308:2;5585/chr17:2173:chr17:43037477-43037308:1;5586/chr5:3644:chr5:178004014-17
8003841:2;5587/chr5:3644:chr5:178004014-178003841:1;5588/chr18:97:chr18:5630216-5630088:2;5589/chr18:
97:chr18:5630216-5630088:1;5590/chr5:2195:chr5:127873613-127873456:2;5591/chr6:95:chr6:1379195-1379
023:2;5592/chr9:58:chr9:1050317-1050143:2;5593/chr9:58:chr9:1050317-1050143:1;5594/chr8:2251:chr8:9998
6186-99986014:2;5595/chr8:2251:chr8:99986186-99986014:1;5596/chr11:716:chr11:2291863-2291733:2;5597/
chr11:716:chr11:2291863-2291733:1;5598/chr8:656:chr8:10590357-10590187:2;5599/chr8:656:chr8:10590357-
10590187:1;5600/chr15:2501:chr15:101513808-101513644:2;5601/chr15:2501:chr15:101513808-101513644:1;
5602/chr9:2786:chr9:137348062-137347894:2;5603/chr9:2786:chr9:137348062-137347894:1;5604/chr5:3119:c
hr5:170737150-170736991:2;5605/chr5:3119:chr5:170737150-170736991:1;5606/chr5:2195:chr5:127873613-1
27873456:1;5607/chr6:95:chr6:1379195-1379023:1;5608/chr5:2324:chr5:134363772-134363647:2;5609/chr5:2
324:chr5:134363772-134363647:1;5610/chr13:1053:chr13:100548537-100548372:2;5611/chr13:1053:chr13:10
0548537-100548372:1;5612/chr13:1076:chr13:100641969-100641838:2;5613/chr13:1076:chr13:100641969-10
0641838:1;5614/chr4:1602:chr4:37245765-37245639:2;5615/chr4:1602:chr4:37245765-37245639:1;5616/chr7:
1084:chr7:4922855-4922683:2;5617/chr7:1084:chr7:4922855-4922683:1;5618/chr16:3098:chr16:73100670-731
00502:2;5619/chr16:3098:chr16:73100670-73100502:1;5620/chr20:946:chr20:32857163-32856988:2;5621/chr2
0:946:chr20:32857163-32856988:1;5622/cg12584684:chr4:96471254-96471102:1;5623/chr20:924:chr20:32254
695-32254524:2;5624/chr20:924:chr20:32254695-32254524:1;5625/chr6:2544:chr6:137244943-137244813:2;5
626/chr6:2544:chr6:137244943-137244813:1;5627/chrX:132:chrX:933906-933732:2;5628/chrX:132:chrX:93390
6-933732:1;5629/chr19:3094:chr19:36246781-36246606:2;5630/chr19:3094:chr19:36246781-36246606:1;5631/
chr22:1894:chr22:46659169-46659009:2;5632/chr22:1894:chr22:46659169-46659009:1;5633/chr2:1902:chr2:6
3275075-63274907:2;5634/chr2:1902:chr2:63275075-63274907:1;5635/chr22:1790:chr22:46263325-46263195:
2;5636/chr22:1790:chr22:46263325-46263195:1;5637/chr6:21:chr6:392249-392123:2;5638/chr6:21:chr6:39224
9-392123:1;5639/chr16:375:chr16:1203102-1202962:2;5640/chr16:375:chr16:1203102-1202962:1;5641/chr19:2
032:chr19:17439716-17439561:2;5642/chr19:2032:chr19:17439716-17439561:1;5643/chr16:4109:chr16:88497
263-88497133:2;5644/chr16:4109:chr16:88497263-88497133:1;5645/chr4:56:chr4:675886-675711:2;5646/chr2:
4739:chr2:219866463-219866334:2;5647/chr22:14:chr22:17082729-17082562:2;5648/chr22:14:chr22:1708272
9-17082562:1;5649/chr16:1606:chr16:23847846-23847674:2;5650/chr16:1606:chr16:23847846-23847674:1;56
51/chr18:1613:chr18:76741126-76740961:2;5652/chr18:1613:chr18:76741126-76740961:1;5653/chr8:2113:chr
8:91803804-91803648:2;5654/chr8:2113:chr8:91803804-91803648:1;5655/chr9:376:chr9:35675734-35675563:
2;5656/chr9:376:chr9:35675734-35675563:1;5657/chr22:2727:chr22:50737950-50737780:2;5658/chr22:2727:c
hr22:50737950-50737780:1;5659/chr16:4191:chr16:88701522-88701378:2;5660/chr16:4191:chr16:88701522-8
8701378:1;5661/chr5:2254:chr5:132161908-132161783:2;5662/chr5:3395:chr5:176024006-176023831:2;5663/
chr5:3395:chr5:176024006-176023831:1;5664/chr18:487:chr18:22929814-22929644:2;5665/chr18:487:chr18:2
2929814-22929644:1;5666/chr10:2698:chr10:102587158-102586988:1;5667/chr12:1171:chr12:52652665-5265
2534:2;5668/chr12:1171:chr12:52652665-52652534:1;5669/chr5:2559:chr5:140871691-140871518:2;5670/chr5:
2559:chr5:140871691-140871518:1;5671/chr15:1198:chr15:68119606-68119446:2;5672/chr15:1198:chr15:681
19606-68119446:1;5673/chr22:161:chr22:19711058-19710889:2;5674/chr22:161:chr22:19711058-19710889:1;
5675/cg04992638:chr17:43339363-43339193:2;5676/cg04992638:chr17:43339363-43339193:1;5677/chr4:241:
chr4:1537998-1537849:1;5678/chr1:7360:chr1:246952511-246952336:2;5679/chr1:7360:chr1:246952511-2469
52336:1;5680/chr2:1990:chr2:66672616-66672448:2;5681/chr2:1990:chr2:66672616-66672448:1;5682/chr18:1
046:chr18:56941474-56941302:2;5683/chr18:1046:chr18:56941474-56941302:1;5684/chr18:994:chr18:550950
00-55094875:2;5685/chr18:994:chr18:55095000-55094875:1;5686/chr14:411:chr14:38724586-38724453:2;568
7/chr14:411:chr14:38724586-38724453:1;5688/chr21:59:chr21:19617260-19617093:2;5689/chr21:59:chr21:196
17260-19617093:1;5690/chr2:5240:chr2:237078678-237078505:2;5691/chr2:5240:chr2:237078678-237078505:
1;5692/chr12:3051:chr12:130388125-130387982:2;5693/chr12:3051:chr12:130388125-130387982:1;5694/chr9:
1343:chr9:110250320-110250162:2;5695/chr9:1343:chr9:110250320-110250162:1;5696/chr5:3993:chr5:18048
6505-180486361:2;5697/chr5:3993:chr5:180486505-180486361:1;5698/chr5:2254:chr5:132161908-132161783:
1;5699/chr7:4153:chr7:150812462-150812321:2;5700/chr7:4153:chr7:150812462-150812321:1;5701/chr4:1685:
chr4:41869429-41869262:2;5702/chr4:1685:chr4:41869429-41869262:1;5703/chr13:1468:chr13:112727628-11
2727494:2;5704/chr13:1468:chr13:112727628-112727494:1;5705/chr10:801:chr10:17496568-17496394:2;5706/
chr10:801:chr10:17496568-17496394:1;5707/chr11:141:chr11:849848-849719:2;5708/chr11:141:chr11:849848-
849719:1;5709/chr12:1313:chr12:54423627-54423453:2;5710/chr12:1313:chr12:54423627-54423453:1;5711/c
hr22:1367:chr22:42679541-42679383:2;5712/chr22:1367:chr22:42679541-42679383:1;5713/chr6:890:chr6:295
21475-29521327:2;5714/chr6:890:chr6:29521475-29521327:1;5715/chr2:1220:chr2:25439236-25439089:2;571
6/chr2:1220:chr2:25439236-25439089:1;5717/chr20:2210:chr20:59827942-59827802:2;5718/chr20:2210:chr20:
59827942-59827802:1;5719/chr3:20:chr3:2140692-2140567:2;5720/chr3:20:chr3:2140692-2140567:1;5721/chr
17:4566:chr17:79919939-79919807:2;5722/chr17:4566:chr17:79919939-79919807:1;5723/chr7:3763:chr7:1391
68029-139167878:2;5724/chr7:3763:chr7:139168029-139167878:1;5725/chr3:2394:chr3:128274309-12827414
9:2;5726/chr3:2394:chr3:128274309-128274149:1;5727/chr11:4527:chr11:126286718-126286556:2;5728/chr11:
4527:chr11:126286718-126286556:1;5729/chr2:4224:chr2:176957722-176957553:2;5730/chr2:4224:chr2:1769
57722-176957553:1;5731/chr1:7209:chr1:237205651-237205479:2;5732/chr1:7209:chr1:237205651-23720547
9:1;5733/chr13:874:chr13:79177711-79177558:2;5734/chr13:874:chr13:79177711-79177558:1;5735/chr5:3113:
chr5:170289726-170289592:2;5736/chr5:3113:chr5:170289726-170289592:1;5737/chr14:1682:chr14:97499691-
97499557:2;5738/chr14:1682:chr14:97499691-97499557:1;5739/chr5:2254:chr5:132161839-132161673:1;574
0/chr20:2210:chr20:59827590-59827450:2;5741/chr20:2210:chr20:59827590-59827450:1;5742/chr10:4059:chr
10:131763512-131763357:2;5743/chr10:4059:chr10:131763512-131763357:1;5744/chr10:861:chr10:22542665-
22542525:2;5745/chr10:861:chr10:22542665-22542525:1;5746/chr16:726:chr16:2835707-2835557:2;5747/chr1
6:726:chr16:2835707-2835557:1;5748/chr15:753:chr15:4547971745479584:2;5749/chr15:753:chr15:45479717-
45479584:1;5750/chr7:735:chr7:2558495-2558331:2;5751/chr7:735:chr7:2558495-2558331:1;5752/chr4:56:chr
4:675886-675711:1;5753/chr2:4739:chr2:219866463-219866334:1;5754/chr4:391:chr4:2042346-2042172:2;575
5/chr14:931:chr14:72399453-72399302:2;5756/chr14:931:chr14:72399453-72399302:1;5757/chr5:2325:chr5:13
4364785-134364610:2;5758/chr5:2325:chr5:134364785-134364610:1;5759/chr4:391:chr4:2042346-2042172:1;
5760/chr14:2078:chr14:102027529-102027357:2;5761/chr14:2078:chr14:102027529-102027357:1;5762/chr1:3
004:chr1:25257252-25257112:2;5763/chr1:3004:chr1:25257252-25257112:1;5764/chr5:3113:chr5:170289076-1
70288926:2;5765/chr5:3113:chr5:170289076-170288926:1;5766/chr2:1992:chr2:66673340-66673170:2;5767/c
hr2:1992:chr2:66673340-66673170:1;5768/chr15:1842:chr15:84322769-84322594:2;5769/chr15:1842:chr15:84
322769-84322594:1;5770/chr10:583:chr10:11059779-11059606:2;5771/chr10:583:chr10:11059779-11059606:1;
5772/chr18:637:chr18:35065518-35065382:2;5773/chr18:637:chr18:35065518-35065382:1;5774/chr11:69:chr1
1:504566-504396:2;5775/chr11:69:chr11:504566-504396:1;5776/chr15:628:chr15:41787984-41787836:2;5777/
chr15:628:chr15:41787984-41787836:1;5778/chr11:141:chr11:850072-849924:2;5779/chr11:141:chr11:850072-
849924:1;5780/chr15:207:chr15:27112810-27112675:2;5781/chr15:207:chr15:27112810-27112675:1;5782/chr7:
3616:chr7:130418851-130418709:2;5783/chr7:3616:chr7:130418851-130418709:1;5784/chr18:768:chr18:4452
7124-44526983:2;5785/chr18:768:chr18:44527124-44526983:1;5786/chr7:3489:chr7:126988181-126988007:2;
5787/chr7:3489:chr7:126988181-126988007:1;5788/chr10:3445:chr10:124896628-124896472:2;5789/chr13:14
67:chr13:112727318-112727168:2;5790/chr13:1467:chr13:112727318-112727168:1;5791/chr18:1835:chr18:77
548155-77548011:2;5792/chr18:1835:chr18:77548155-77548011:1;5793/chr20:2212:chr20:59829028-5982885
4:2;5794/chr20:2212:chr20:59829028-59828854:1;5795/chr10:3445:chr10:124896628-124896472:1;5796/chr7:
2148:chr7:49815639-49815507:2;5797/chr7:2148:chr7:49815639-49815507:1;5798/chr2:4772:chr2:220313181-
220313025:2;5799/chr2:4772:chr2:220313181-220313025:1;5800/chr8:1695:chr8:53852326-53852151:2;5801/
chr8:1695:chr8:53852326-53852151:1;5802/cg00817367:chr12:52401280-52401118:1;5803/chr4:202:chr4:139
9087-1398919:2;5804/chr4:202:chr4:1399087-1398919:1;5805/chr20:2928:chr20:62119708-62119535:2;5806/c
hr20:2928:chr20:62119708-62119535:1;5807/chr17:3502:chr17:73749971-73749845:2;5808/chr17:3502:chr17:
73749971-73749845:1;5809/chr1:4054:chr1:50888835-50888704:2;5810/chr1:4054:chr1:50888835-50888704:1;
5811/chr3:2434:chr3:128764700-128764537:2;5812/chr3:2434:chr3:128764700-128764537:1;5813/chr19:56:c
hr19:519287-519126:2;5814/chr19:56:chr19:519287-519126:1;5815/chr19:1577:chr19:11689915-11689741:2;5
816/chr19:1577:chr19:11689915-11689741:1;5817/cg22538054:chr12:95942041-95941906:2;5818/cg2253805
4:chr12:95942041-95941906:1;5819/chr17:4420:chr17:79315133-79314962:2;5820/chr17:4420:chr17:7931513
3-79314962:1;5821/chr7:3500:chr7:127671286-127671159:2;5822/chr7:3500:chr7:127671286-127671159:1;58
23/chr20:55:chr20:826016-825843:2;5824/chr20:55:chr20:826016-825843:1;5825/chr3:1158:chr3:48699245-48
699071:2;5826/chr3:1158:chr3:48699245-48699071:1;5827/chr9:1070:chr9:96714495-96714322:2;5828/chr9:1
070:chr9:96714495-96714322:1;5829/chr15:1200:chr15:68120586-68120418:2;5830/chr16:2511:chr16:553585
67-55358398:2;5831/chr16:2511:chr16:55358567-55358398:1;5832/chr19:267:chr19:1169158-1169009:2;5833/
chr19:267:chr19:1169158-1169009:1;5834/chr15:1200:chr15:68120586-68120418:1;5835/chr17:2307:chr17:45
786578-45786437:2;5836/chr17:2307:chr17:45786578-45786437:1;5837/chr1:5084:chr1:145562958-14556278
6:2;5838/chr1:5084:chr1:145562958-145562786:1;5839/cg21938148:chr13:110959065-110958892:1;5840/chr1:
5027:chr1:119533005-119532832:2;5841/chr1:5027:chr1:119533005-119532832:1;5842/cg05238517:chr11:10
4034369-104034195:1;5843/chr1:6195:chr1:203045304-203045137:2;5844/chr1:6195:chr1:203045304-203045
137:1;5845/chr16:1850:chr16:29888266-29888127:2;5846/chr16:1850:chr16:29888266-29888127:1;5847/chr1:
4908:chr1:113052004-113051829:1;5848/chr2:4772:chr2:220313324-220313176:2;5849/chr2:4772:chr2:22031
3324-220313176:1;5850/chr13:1782:chr13:114524069-114523894:2;5851/chr13:1782:chr13:114524069-11452
3894:1;5852/chr12:1171:chr12:52652387-52652216:2;5853/chr12:1171:chr12:52652387-52652216:1;5854/chr1:
3659:chr1:39957476-39957306:2;5855/chr1:3659:chr1:39957476-39957306:1;5856/chr15:2253:chr15:9538858
8-95388417:2;5857/chr15:2253:chr15:95388588-95388417:1;5858/chr7:1533:chr7:27205311-27205186:2;5859/
chr7:1533:chr7:27205311-27205186:1;5860/chr18:1140:chr18:67068679-67068508:2;5861/chr18:1140:chr18:6
7068679-67068508:1;5862/chr4:2981:chr4:189203881-189203706:2;5863/chr4:2981:chr4:189203881-1892037
06:1;5864/chr20:144:chr20:2732755-2732582:2;5865/chr20:144:chr20:2732755-2732582:1;5866/chr5:1826:chr
5:77806081-77805956:2;5867/chr5:1826:chr5:77806081-77805956:1;5868/cg06995920:chr12:64062619-64062
459:2;5869/cg04455164:chr12:64062619-64062459:2;5870/cg06995920:chr12:64062619-64062459:1;5871/cg
04455164:chr12:64062619-64062459:1;5872/chr17:1113:chr17:14212620-14212467:2;5873/chr17:1113:chr17:
14212620-14212467:1;5874/chr18:577:chr18:32073936-32073761:2;5875/chr18:577:chr18:32073936-3207376
1:1;5876/cg02526522:chr11:128563749-128563576:1;5877/chr18:723:chr18:43914226-43914053:2;5878/chr18:
723:chr18:43914226-43914053:1;5879/chr22:141:chr22:19512332-19512165:2;5880/chr22:141:chr22:1951233
2-19512165:1;5881/chr6:1002:chr6:31466008-31465838:2;5882/chr6:1002:chr6:31466008-31465838:1;5883/ch
rX:810:chrX:48685208-48685047:2;5884/chrX:810:chrX:48685208-48685047:1;5885/chr18:77:chr18:4455361-4
455197:2;5886/chr18:77:chr18:4455361-4455197:1;5887/chr15:1417:chr15:74426087-74425915:2;5888/chr15:
1417:chr15:74426087-74425915:1;5889/chr19:3621:chr19:42704048-42703889:2;5890/chr19:3621:chr19:4270
4048-42703889:1;5891/chr18:1044:chr18:56940665-56940493:2;5892/chr18:1044:chr18:56940665-56940493:1;
5893/chr6:95:chr6:1378815-1378642:2;5894/chr6:95:chr6:1378815-1378642:1;5895/chr10:629:chr10:1178466
1-11784500:2;5896/chr10:629:chr10:11784661-11784500:1;5897/chr18:1360:chr18:74962761-74962636:2;589
8/chr18:1360:chr18:74962761-74962636:1;5899/chr3:2978:chr3:170137642-170137498:2;5900/chr3:2978:chr3:
170137642-170137498:1;5901/chr7:4691:chr7:156796899-156796731:2;5902/chr7:4691:chr7:156796899-1567
96731:1;5903/chr14:578:chr14:57264923-57264748:2;5904/chr14:578:chr14:57264923-57264748:1;5905/chr19:
3999:chr19:48947166-48947021:2;5906/chr19:3999:chr19:48947166-48947021:1;5907/chr14:626:chr14:59931
541-59931381:2;5908/chr14:626:chr14:59931541-59931381:1;5909/chr4:15:chr4:330723-330592:2;5910/chr4:1
5:chr4:330723-330592:1;5911/chr17:4140:chr17:77789621-77789455:2;5912/chr17:4140:chr17:77789621-7778
9455:1;5913/chr19:2639:chr19:30866152-30865978:2;5914/chr19:2639:chr19:30866152-30865978:1;5915/cg0
1163842:chr14:95235193-95235024:2:5916/cg01163842:chr14:95235193-95235024:1;5917/chr1:4827:chr1:11
0613551-110613426:1;5918/chr17:3065:chr17:70112144-70111978:2;5919/chr17:3065:chr17:70112144-70111
978:1;5920/chr2:1230:chr2:25500053-25499880:2;5921/chr2:1230:chr2:25500053-25499880:1;5922/chr5:1861:
chr5:79866002-79865832:2;5923/chr6:1861:chr5:79866002-79865832:1;5924/cg04261408:chr8:97506712-975
06541:1;5925/chr21:324:chr21:38076912-38076760:2;5926/chr21:324:chr21:38076912-38076760:1;5927/chr8:
1962:chr8:70982068-70981941:2;5928/chr8:1962:chr8:70982068-70981941:1;5929/chr15:1205:chr15:6812242
9-68122260:2;5930/chr15:1205:chr15:68122429-68122260:1;5931/chr3:3248:chr3:186490814-186490642:2;59
32/chr3:3248:chr3:186490814-186490642:1;5933/cg24446548:chr7:19157307-19157173:2;5934/cg24446548:c
hr7:19157307-19157173:1;5935/chr15:2554:chr15:102029932-102029775:2;5936/chr15:2554:chr15:102029932-
102029775:1;5937/chr22:14:chr22:17082836-17082687:2;5938/chr22:14:chr22:17082836-17082687:1;5939/ch
r5:2729:chr5:149546693-149546549:2;5940/chr5:2729:chr5:149546693-149546549:1;5941/chr11:2620:chr11:6
6885832-66885684:2;5942/chr11:2620:chr11:66885832-66885684:1;5943/chr19:2895:chr19:34112897-341127
45:2;5944/chr19:2895:chr19:34112897-34112745:1;5945/chr5:2450:chr5:138730244-138730099:2;5946/chr19:
2031:chr19:17439281-17439106:2;5947/chr19:2031:chr19:17439281-17439106:1;5948/chr19:367:chr19:14680
98-1467932:2;5949/chr19:367:chr19:1468098-1467932:1;5950/chr5:2450:chr5:138730244-138730099:1;5951/c
hr22:2205:chr22:48971511-48971359:2;5952/chr22:2205:chr22:48971511-48971359:1;5953/chr15:428:chr15:3
1776109-31775934:2;5954/chr15:428:chr15:31776109-31775934:1;5955/chr18:989:chr18:55019875-55019712:
1;5956/chr4:207:chr4:1400597-1400431:2;5957/chr4:207:chr4:1400597-1400431:1;5958/chr8:2113:chr8:91803
995-91803870:2;5959/chr8:2113:chr8:91803995-91803870:1;5960/chr15:2003:chr15:89922598-89922472:2;59
61/chr2:4079:chr2:171570360-171570195:2;5962/chr2:4079:chr2:171570360-171570195:1;5963/chr8:2284:chr
8:102506246-102506076:2;5964/chr8:2284:chr8:102506246-102506076:1;5965/chr1:2566:chr1:18434439-1843
4311:2;5966/chr1:2566:chr1:18434439-18434311:1;5967/chr19:392:chr19:1524388-1524256:2;5968/chr19:392:
chr19:1524388-1524256:1;5969/chr2:5231:chr2:237072463-237072312:2;5970/chr2:5231:chr2:237072463-237
072312:1;5971/chr18:1125:chr18:63418321-63418149:2;5972/chr18:1125:chr18:63418321-63418149:1;5973/c
hr15:2003:chr15:89922598-89922472:1;5974/chr1:6544:chr1:214153411-214153267:2;5975/chr1:6544:chr1:21
4153411-214153267:1;5976/chr10:784:chr10:16562384-16562252:2;5977/chr10:784:chr10:16562384-1656225
2:1;5978/chr17:176:chr17:1174133-1173958:2;5979/chr17:176:chr17:1174133-1173958:1;5980/chr18:1140:chr
18:67068305-67068162:2;5981/chr18:1140:chr18:67068305-67068162:1;5982/chr19:3278:chr19:38886596-388
86453:2;5983/chr19:3278:chr19:38886596-38886453:1;5984/chr22:2146:chr22:48885541-48885378:2;5985/chr
22:2146:chr22:48885541-48885378:1;5986/chr10:2350:chr10:88728396-88728260:2;5987/chr2:5862:chr2:2424
98651-242498479:2;5988/chr2:5862:chr2:242498651-242498479:1;5989/chr6:138:chr6:1615177-1615052:2;59
90/chr6:138:chr6:1615177-1615052:1;5991/chr12:3095:chr12:130647507-130647367:2;5992/chr12:3095:chr12:
130647507-130647367:1;5993/chr14:627:chr14:59931992-59931831:2;5994/chr14:627:chr14:59931992-59931
831:1;5995/chr2:5862:chr2:242498721-242498552:2;5996/chr2:5862:chr2:242498721-242498552:1;5997/chr8:
1921:chr8:67873665-67873500:2;5998/chr8:1921:chr8:67873665-67873500:1;5999/chr10:2350:chr10:8872839
6-88728260:1;6000/chr7:3616:chr7:130419074-130418920:1;6001/chr5:514:chr5:1887499-1887339:2;6002/chr
5:514:chr5:1887499-1887339:1;6003/chr5:3347:chr5:175224103-175223936:2;6004/chr5:3347:chr5:175224103-
175223936:1;6005/chr4:15:chr4:330537-330407:2;6006/chr4:15:chr4:330537-330407:1;6007/chr10:2401:chr10:
94449835-94449662:2;6008/chr10:2401:chr10:94449835-94449662:1;6009/chr20:764:chr20:26189186-261890
11:2;6010/chr20:764:chr20:26189186-26189011:1;6011/chr19:3890:chr19:47153247-47153075:2;6012/chr19:3
890:chr19:47153247-47153075:1;6013/chr1:5286:chr1:154475470-154475330:2;6014/chr1:5286:chr1:1544754
70-154475330:1;6015/chr20:765:chr20:26189874-26189717:2;6016/chr20:765:chr20:26189874-26189717:1;60
17/chr19:1577:chr19:11689972-11689797:1;6018/chr2:3108:chr2:115919832-115919657:2;6019/chr2:3108:chr
2:115919832-115919657:1;6020/chr17:2060:chr17:40822267-40822117:2;6021/chr17:2060:chr17:40822267-40
822117:1;6022/chr13:877:chr13:79183841-79183703:2;6023/chr13:877:chr13:79183841-79183703:1;6024/chr9:
2117:chr9:132382632-132382477:2;6025/chr9:2117:chr9:132382632-132382477:1;6026/chr10:2358:chr10:903
43204-90343030:2;6027/chr10:2358:chr10:90343204-90343030:1;6028/chr7:3501:chr7:127671862-127671704:
2;6029/chr7:3501:chr7:127671862-127671704:1;6030/chr12:428:chr12:6665108-6664937:1;6031/chr9:60:chr9:
1051814-1051639:2;6032/chr9:60:chr9:1051814-1051639:1;6033/chr1:6194:chr1:203044899-203044759:2;603
4/chr1:6194:chr1:203044899-203044759:1;6035/chr2:1560:chr2:42720506-42720356:2;6036/chr2:1560:chr2:42
720506-42720356:1;6037/chr19:2895:chr19:34113030-34112858:2;6038/chr19:2895:chr19:34113030-3411285
8:1;6039/chr14:586:chr14:57276035-57275860:2;6040/chr14:586:chr14:57276035-57275860:1;6041/chr6:21:ch
r6:392409-392234:2;6042/chr6:21:chr6:392409-392234:1;6043/chr4:2032:chr4:81124206-81124049:2;6044/chr
4:2032:chr4:81124206-81124049:1;6045/chr5:3113:chr5:170289370-170289203:1;6046/chr8:354:chr8:1950507-
1950350:2;6047/chr8:354:chr8:1950507-1950350:1;6048/chr3:2636:chr3:138664580-138664436:2;6049/chr3:2
636:chr3:138664580-138664436:1;6050/chr10:861:chr10:22542355-22542187:2;6051/chr10:861:chr10:225423
55-22542187:1;6052/chr7:2589:chr7:70596977-70596816:2;6053/chr7:2589:chr7:70596977-70596816:1;6054/c
hr4:1602:chr4:37245890-37245755:2;6055/chr4:1602:chr4:37245890-37245755:1;6056/chr11:104:chr11:63968
7-639520:2;6057/chr2:4186:chr2:175201381-175201219:2;6058/chr2:4186:chr2:175201381-175201219:1;6059/
chr11:104:chr11:639687-639520:1;6060/chr7:3975:chr7:149119747-149119596:2;6061/chr7:3975:chr7:149119
747-149119596:1;6062/chr20:1816:chr20:55206283-55206143:2;6063/chr20:1816:chr20:55206283-55206143:1;
6064/chr10:2403:chr10:94450443-94450308:2;6065/chr10:2403:chr10:94450443-94450308:1;6066/chr8:1788:
chr8:57358471-57358339:2;6067/chr8:1788:chr8:57358471-57358339:1;6068/chr6:2396:chr6:117869225-1178
69068:2;6069/chr6:2396:chr6:117869225-117869068:1;6070/chr7:1519:chr7:27195776-27195601:2;6071/chr7:
1519:chr7:27195776-27195601:1;6072/chr19:322:chr19:1295724-1295549:2;6073/chr19:322:chr19:1295724-12
95549:1;6074/chr1:1852:chr1:8277718-8277568:2;6075/chr1:1852:chr1:8277718-8277568:1;6076/chr19:553:ch
r19:2252101-2251946:2;6077/chr19:553:chr19:2252101-2251946:1;6078/chr2:3074:chr2:114035004-11403484
3:2;6079/chr4:2973:chr4:188916952-188916798:2;6080/chr18:723:chr18:43913923-43913761:2;6081/chr18:72
3:chr18:43913923-43913761:1;6082/chr2:3074:chr2:114035004-114034843:1;6083/chr4:2973:chr4:188916952-
188916798:1;6084/chr2:2631:chr2:99439114-99438962:2;6085/chr2:2631:chr2:99439114-99438962:1;6086/chr
17:1867:chr17:36718302-36718143:2;6087/chr17:1867:chr17:36718302-36718143:1;6088/chr11:461:chr11:186
3973-1863822:2;6089/chr11:461:chr11:1863973-1863822:1;6090/chr1:5137:chr1:150522174-150522001:2;609
1/chr1:5137:chr1:150522174-150522001:1;6092/chr12:3052:chr12:130388764-130388596:2;6093/chr12:3052:c
hr12:130388764-130388596:1;6094/cg10512745:chr1:50884503-50884345:2;6095/cg10512745:chr1:50884503-
50884345:1;6096/chr1:1360:chr1:4221643-4221477:2;6097/chr1:1360:chr1:4221643-4221477:1;6098/chr19:49
04:chr19:58570654-58570520:2;6099/chr19:4904:chr19:58570654-58570520:1;6100/chr22:369:chr22:2286291
6-22862766:2;6101/chr22:369:chr22:22862916-22862766:1;6102/chr18:637:chr18:35065417-35065275:2;6103/
chr18:637:chr18:35065417-35065275:1;6104/chr6:380:chr6:10382369-10382195:2;6105/chr4:1265:chr4:88603
99-8860227:2:6106/chr1:255:chr1:1567521-1567349:2;6107/chr1:255:chr1:1567521-1567349:1;6108/chr5:1172:
chr5:15500589-15500424:2;6109/chr1:3539:chr1:37499966-37499814:2;6110/chr1:3539:chr1:37499966-37499
814:1;6111/chr2:4945:chr2:231693374-231693217:2;6112/chr2:4945:chr2:231693374-231693217:1;6113/chr5:
1172:chr5:15500589-15500424:1;6114/chr14:578:chr14:57265238-57265091:2;6115/chr14:578:chr14:5726523
8-57265091:1;6116/chr19:3157:chr19:37096331-37096197:1;6117/chr5:1726:chr5:72733070-72732898:2;6118/
chr5:1726:chr5:72733070-72732898:1;6119/chr1:1408:chr1:4715443-4715288:2;6120/chr1:1408:chr1:4715443-
4715288:1;6121/chr7:2162:chr7:50344065-50343906:2;6122/chr7:2162:chr7:50344065-50343906:1;6123/chr6:
380:chr6:10382369-10382195:1;6124/chr4:1265:chr4:8860399-8860227:1;6125/chr16:2882:chr16:67428310-67
428160:2;6126/chr16:2882:chr16:67428310-67428160:1;6127/chr19:4944:chr19:58907970-58907799:2;6128/c
hr19:4944:chr19:58907970-58907799:1;6129/chr8:2786:chr8:140716132-140715978:2;6130/chr8:2786:chr8:14
0716132-140715978:1;6131/chr16:2379:chr16:51168402-51168277:2;6132/chr16:2379:chr16:51168402-51168
277:1;6133/chr1:645:chr1:2706435-2706275:2;6134/chr1:645:chr1:2706435-2706275:1;6135/chr4:2081:chr4:85
414500-85414335:2;6136/chr4:2081:chr4:85414500-85414335:1;6137/chr8:2241:chr8:99960687-99960533:2;6
138/chr12:2900:chr12:127630608-127630479:2;6139/chr12:2900:chr12:127630608-127630479:1;6140/chr8:22
41:chr8:99960687-99960533:1;6141/chr8:2717:chr8:139509484-139509342:2;6142/chr8:2717:chr8:139509484-
139509342:1;6143/chr20:1322:chr20:43727417-43727246:2;6144/chr20:1322:chr20:43727417-43727246:1;614
5/chr20:620:chr20:23029334-23029165:2;6146/chr20:620:chr20:23029334-23029165:1;6147/chr6:852:chr6:289
45313-28945142:2;6148/chr6:852:chr6:28945313-28945142:1;6149/chr5:2522:chr5:140306339-140306167:2;6
150/chr5:2522:chr5:140306339-140306167:1;6151/chr2:4287:chr2:177043089-177042954:2;6152/chr2:4287:ch
r2:177043089-177042954:1;6153/chr20:555:chr20:21503486-21503361:2;6154/chr20:555:chr20:21503486-215
03361:1;6155/chr17:2653:chr17:54756036-54755893:2;6156/chr17:2653:chr17:54756036-54755893:1;6157/chr
6:138:chr6:1614662-1614512:2;6158/chr6:138:chr6:1614662-1614512:1;6159/chrX:693:chrX:40005438-400052
93:2;6160/chr15:2011:chr15:89950925-89950795:2;6161/chr15:2011:chr15:89950925-89950795:1;6162/chr6:1
985:chr6:85484265-85484099:2;6163/chr6:1985:chr6:85484265-85484099:1;6164/chr5:3916:chr5:180018487-1
80018326:2;6165/chr5:3916:chr5:180018487-180018326:1;6166/chr1:3805:chr1:44031780-44031655:2;6167/c
hr1:3805:chr1:44031780-44031655:1;6168/chrX:693:chrX:40005438-40005293:1;6169/chr10:2024:chr10:77156
888-77156723:2;6170/chr10:2024:chr10:77156888-77156723:1;6171/chr17:2653:chr17:54756160-54755985:2;
6172/chr17:2653:chr17:54756160-54755985:1;6173/chr8:42:chr8:689358-689185:1;6174/chr14:212:chr14:2706
7365-27067213:2;6175/chr21:948:chr21:45875566-45875420:2;6176/chr21:948:chr21:45875566-45875420:1;6
177/chr14:212:chr14:27067365-27067213:1;6178/chr6:2544:chr6:137244816-137244641:2;6179/chr6:2544:chr
6:137244816-137244641:1;6180/chr6:2392:chr5:135266425-135266290:2;6181/chr5:2392:chr5:135266425-135
266290:1;6182/chr7:4146:chr7:150778929-150778781:1;6183/chr10:1587:chr10:52178191-52178042:2;6184/c
hr10:1587:chr10:52178191-52178042:1;6185/chr16:726:chr16:2835652-2835510:2;6186/chr16:726:chr16:2835
652-2835510:1;6187/chr11:2620:chr11:66885299-66885163:2;6188/chr11:2620:chr11:66885299-66885163:1;6
189/chr12:3619:chr12:133484966-133484792:2;6190/chr6:245:chr6:4776211-4776040:2;6191/chr6:245:chr6:47
76211-4776040:1;6192/chr10:905:chr10:23480998-23480833:2;6193/chr10:905:chr10:23480998-23480833:1;6
194/chr8:37:chr8:687143-686981:2;6195/chr8:37:chr8:687143-686981:1;6196/chr20:1878:chr20:55965213-559
65073:2;6197/chr20:1878:chr20:55965213-55965073:1;6198/chr5:91:chr5:528981-528856:2;6199/chr5:91:chr5:
528981-528856:1;6200/chr9:1427:chr9:116225797-116225647:2;6201/chr9:1427:chr9:116225797-116225647:1;
6202/chr14:1633:chr14:96343500-96343352:2;6203/chr14:1633:chr14:96343500-96343352:1;6204/chr2:605:c
hr2:5837435-5837276:2;6205/chr2:605:chr2:5837435-5837276:1;6206/chr9:1388:chr9:113341965-113341810:
1;6207/chr2:4215:chr2:176945388-176945213:2;6208/chr2:4215:chr2:176945388-176945213:1;6209/chr17:277
8:chr17:58227558-58227398:2;6210/chr17:2778:chr17:58227558-58227398:1;6211/chr12:3619:chr12:1334849
66-133484792:1;6212/chr18:765:chr18:44338048-44337892:2;6213/chr18:765:chr18:44338048-44337892:1;62
14/chr1:3805:chr1:44031658-44031483:2;6215/chr1:3805:chr1:44031658-44031483:1;6216/chr14:92:chr14:238
35017-23834845:2;6217/chr14:92:chr14:23835017-23834845:1;6218/chr1:1254:chr1:3567506-3567358:2;6219/
chr1:1254:chr1:3567506-3567358:1;6220/chr7:4724:chr7:157072394-157072245:2;6221/chr7:4724:chr7:15707
2394-157072245:1;6222/chr7:1307:chr7:19156872-19156722:2;6223/chr7:1307:chr7:19156872-19156722:1;62
24/cg25023275:chr18:35146520-35146345:1;6225/chr1:1594:chr1:6126565-6126409:2;6226/chr1:1594:chr1:61
26565-6126409:1;6227/chr16:3098:chr16:73100355-73100229:2;6228/chr16:3098:chr16:73100355-73100229:1;
6229/chr2:4263:chr2:176995352-176995189:2;6230/chr2:4263:chr2:176995352-176995189:1;6231/chr20:2966:
chr20:62273016-62272890:2;6232/chr20:2966:chr20:62273016-62272890:1;6233/chr6:789:chr6:27725448-277
25301:1;6234/chr2:4131:chr2:172965313-172965138:2;6235/chr2:4131:chr2:172965313-172965138:1;6236/chr
13:236:chr13:25320484-25320348:2;6237/chr14:2086:chr14:102057233-102057077:2;6238/chr14:2086:chr14:1
02057233-102057077:1;6239/chr1:3709:chr1:41327944-41327808:2;6240/chr1:3709:chr1:41327944-41327808:
1;6241/chr13:236:chr13:25320484-25320348:1;6242/chr22:1094:chr22:38478958-38478822:2;6243/chr22:1094:
chr22:38478958-38478822:1;6244/chrX:666:chrX:39864599-39864460:2;6245/chrX:666:chrX:39864599-39864
460:1;6246/chr10:4054:chr10:131758148-131757973:2;6247/chr10:4054:chr10:131758148-131757973:1;6248/
cg25363607:chr7:90226416-90226241:1;6249/chr3:3168:chr3:184301860-184301697:2;6250/chr3:3168:chr3:1
84301860-184301697:1;6251/chr8:3703:chr8:145106620-145106487:2;6252/chr8:3703:chr8:145106620-14510
6487:1;6253/chr9:1770:chr9:127265784-127265626:2;6254/chr9:1770:chr9:127265784-127265626:1;6255/chr2:
4217:chr2:176947044-176946900:2;6256/chr2:4217:chr2:176947044-176946900:1;6257/chr1:3767:chr1:43250
967-43250816:2;6258/chr1:3767:chr1:43250967-43250816:1;6259/cg07961994:chr4:93227319-93227144:1;62
60/chr8:13:chr8:494735-494560:2;6261/chr8:13:chr8:494735-494560:1;6262/chr2:1181:chr2:24233315-242331
54:2;6263/chr2:1181:chr2:24233315-24233154:1;6264/chr6:1746:chr6:50818149-50817991:1;6265/chr13:1173:
chr13:109148200-109148038:2;6266/chr13:1173:chr13:109148200-109148038:1;6267/chr11:141:chr11:850248-
850090:2;6268/chr11:141:chr11:850248-850090:1;6269/chr22:1515:chr22:43807700-43807533:2;6270/chr22:1
515:chr22:43807700-43807533:1;6271/chr9:3255:chr9:139024751-139024593:2;6272/chr9:3255:chr9:1390247
51-139024593:1;6273/chr7:1491:chr7:27153337-27153175:2;6274/chr7:1491:chr7:27153337-27153175:1;6275/
chr8:1349:chr8:37555786-37555617:2;6276/chr8:1349:chr8:37555786-37555617:1;6277/chr18:280:chr18:1175
2227-11752099:2;6278/chr18:280:chr18:11752227-11752099:1;6279/chr1:224:chr1:1475854-1475725:2;6280/c
g16601494:chr1:1475854-1475712:2;6281/chr1:224:chr1:1475854-1475725:1;6282/cg16601494:chr1:1475854-
1475712:1;6283/chr6:920:chr6:30039569-30039402:2;6284/chr6:920:chr6:30039569-30039402:1;6285/chr11:3
716:chr11:101454247-101454084:2;6286/chr8:3776:chr8:145806421-145806253:2;6287/chr8:3776:chr8:14580
6421-145806253:1;6288/chr8:766:chr8:12809427-12809289:2;6289/chr8:766:chr8:12809427-12809289:1;6290/
chr7:3501:chr7:127672199-127672024:2;6291/chr7:3501:chr7:127672199-127672024:1;6292/chr17:1522:chr17:
27038993-27038820:2;6293/chr17:1522:chr17:27038993-27038820:1;6294/cg07195011:chr5:11904224-11904
058:2;6295/cg07195011:chr5:11904224-11904058:1;6296/chr10:3172:chr10:118894343-118894208:1;6297/chr
20:620:chr20:23029468-23029328:2;6298/chr20:620:chr20:23029468-23029328:1;6299/chr22:1248:chr22:4005
8031-40057893:2;6300/chr22:1248:chr22:40058031-40057893:1;6301/chr19:69:chr19:591781-591620:1;6302/c
hr9:1682:chr9:126136006-126135843:2;6303/chr9:1682:chr9:126136006-126135843:1;6304/chr11:1212:chr11:
13031153-13031007:2;6305/chr11:1212:chr11:13031153-13031007:1;6306/chr6:1872:chr6:72892478-7289230
7:2;6307/chr6:1872:chr6:72892478-72892307:1;6308/chr13:1461:chr13:112723166-112723012:2;6309/chr13:1
461:chr13:112723166-112723012:1;6310/chr5:2489:chr5:139228042-139227915:2;6311/chr5:2489:chr5:13922
8042-139227915:1;6312/chr1:4177:chr1:54203906-54203736:2;6313/chr1:4177:chr1:54203906-54203736:1;63
14/chr4:2250:chr4:111542182-111542007:2;6315/chr4:2250:chr4:111542182-111542007:1;6316/chr11:1511:ch
r11:31827174-31827003:2;6317/chr11:1511:chr11:31827174-31827003:1;6318/chr10:4868:chr10:135016088-1
35015916:2;6319/chr10:4868:chr10:135016088-135015916:1;6320/chr12:3095:chr12:130647641-130647492:2;
6321/chr12:3095:chr12:130647641-130647492:1;6322/chr8:3701:chr8:145104534-145104389:2;6323/chr1:225
7:chr1:13840011-13839848:2;6324/chr1:2257:chr1:13840011-13839848:1;6325/chr19:368:chr19:1469397-1469
235:2;6326/chr19:368:chr19:1469397-1469235:1;6327/chr8:3701:chr8:145104534-145104389:1;6328/chr1:59:c
hr1:969513-969349:2;6329/chr1:59:chr1:969513-969349:1;6330/chr15:1417:chr15:74425808-74425644:2;6331/
chr15:1417:chr15:74425808-74425644:1;6332/chr11:4765:chr11:131780758-131780627:2;6333/chr11:4765:ch
r11:131780758-131780627:1;6334/chr6:1322:chr6:35992510-35992365:2;6335/chr6:1322:chr6:35992510-3599
2365:1;6336/chr11:2279:chr11:63530421-63530247:2;6337/chr11:2279:chr11:63530421-63530247:1;6338/chr6:
2544:chr6:137245089-137244924:2;6339/chr6:2544:chr6:137245089-137244924:1;6340/chr15:2482:chr15:101
267698-101267531:1;6341/cg02650317:chr20:61809008-61808863:2;6342/cg02650317:chr20:61809008-6180
8863:1;6343/chr16:2476:chr16:54971316-54971144:1;6344/chr21:214:chr21:34442314-34442159:2;6345/chr21:
214:chr21:34442314-34442159:1;6346/chr1:7209:chr1:237205900-237205749:2;6347/chr1:7209:chr1:2372059
00-237205749:1;6348/chr11:3337:chr11:72295636-72295490:2;6349/chr11:3337:chr1172295636-72295490:1;
6350/chr19:2193:chr19:18771246-18771071:2;6351/chr19:2193:chr19:18771246-18771071:1;6352/chr11:105:c
hr11:640382-640240:2;6353/chr11:105:chr11:640382-640240:1;6354/chr2:3708:chr2:131721162-131721037:2;
6355/chr2:3708:chr2:131721162-131721037:1;6356/chr15:275:chr15:29131415-29131248:2;6357/chr15:275:ch
r15:29131415-29131248:1;6358/chr2:4217:chr2:176947194-176947029:2;6359/chr2:4217:chr2:176947194-176
947029:1;6360/chr11:3716:chr11:101454247-101454084:1;6361/chr8:1276:chr8:31498241-31498079:2;6362/c
hr8:1276:chr8:31498241-31498079:1;6363/chr20:946:chr20:32856904-32856747:2;6364/chr20:946:chr20:3285
6904-32856747:1;6365/chr4:1750:chr4:48492966-48492840:2;6366/chr4:1750:chr4:48492966-48492840:1;636
7/chr15:428:chr15:31776188-31776016:2;6368/chr15:428:chr15:31776188-31776016:1;6369/chr17:1867:chr17:
36718249-36718104:2;6370/chr17:1867:chr17:36718249-36718104:1;6371/chr1:1690:chr1:6507314-6507172:2;
6372/chr1:1690:chr1:6507314-6507172:1;6373/chr4:2602:chr4:155411794-155411651:2;6374/chr4:2602:chr4:
155411794-155411651:1;6375/chr5:1190:chr5:16180233-16180062:2;6376/chr5:1190:chr5:16180233-1618006
2:1;6377/chr19:2032:chr19:17439948-17439779:2;6378/chr19:2032:chr19:17439948-17439779:1;6379/chr18:9
89:chr18:55020141-55020003:2;6380/chr18:989:chr18:55020141-55020003:1;6381/chr7:2146:chr7:49815088-4
9814923:1;6382/chr8:1893:chr8:65493822-65493667:2;6383/chr8:1893:chr8:65493822-65493667:1;6384/chr5:
3695:chr5:178421602-178421458:2;6385/chr5:3695:chr5:178421602-178421458:1;6386/chr6:2396:chr6:11786
9279-117869107:2;6387/chr6:2396:chr6:117869279-117869107:1;6388/chr8:3309:chr8:143545608-143545438:
2;6389/chr8:3309:chr8:143545608-143545438:1;6390/chr16:1968:chr16:31489022-31488879:1;6391/chr10:345
7:chr10:124909688-124909513:2;6392/chr10:3457:chr10:124909688-124909513:1;6393/chr22:2759:chr22:509
82999-50982856:2;6394/chr22:2759:chr22:50982999-50982856:1;6395/chr7:3587:chr7:129423126-129422951:
2;6396/chr7:3587:chr7:129423126-129422951:1;6397/chr13:519:chr13:37006460-37006310:2;6398/chr13:519:
chr13:37006460-37006310:1;6399/chr1:5027:chr1:119532865-119532693:1;6400/chr13:1082:chr13:100649872-
100649730:2;6401/chr13:1082:chr13:100649872-100649730:1;6402/chr2:4182:chr2:175199605-175199460:2;
6403/chr2:4182:chr2:175199605-175199460:1;6404/chr3:3517:chr3:196255783-196255613:2;6405/chr3:3517:c
hr3:196255783-196255613:1;6406/chr2:4215:chr2:176945464-176945327:2;6407/chr2:4215:chr2:176945464-1
76945327:1;6408/chr2:1181:chr2:24233424-24233287:2;6409/chr2:1181:chr2:24233424-24233287:1;6410/chr1
0:4582:chr10:134598600-134598471:2;6411/chr10:4582:chr10:134598600-134598471:1;6412/chr11:2420:chr1
1:64428300-64428155:2;6413/chr11:2420:chr11:64428300-64428155:1;6414/chr10:3710:chr10:128077523-128
077394:2;6415/chr10:3710:chr10:128077523-128077394:1;6416/chr8:96:chr8:876946-876781:2;6417/chr8:96:c
hr8:876946-876781:1;6418/chr14:1433:chr14:93153600-93153457:1;6419/chr19:3765:chr19:45900194-459000
30:2;6420/chr19:3765:chr19:45900194-45900030:1;6421/chr19:392:chr19:1524468-1524303:2;6422/chr19:392:
chr19:1524468-1524303:1;6423/chr20:2023:chr20:57464386-57464221:2;6424/chr20:2023:chr20:57464386-57
464221:1;6425/chr10:2027:chr10:77159317-77159146:2;6426/chr10:2027:chr10:77159317-77159146:1;6427/c
hr11:98:chr11:626908-626783:2;6428/chr11:98:chr11:626908-626783:1;6429/chr19:551:chr19:2251616-22514
73:2;6430/chr19:551:chr19:2251616-2251473:1;6431/chr11:2774:chr11:68145477-68145328:1;6432/chr10:291
1:chr10:106401185-106401050:2;6433/chr10:2911:chr10:106401185-106401050:1;6434/chr15:634:chr15:4179
5212-41795045:2;6435/chr15:634:chr15:41795212-41795045:1;6436/chr20:3023:chr20:62463499-62463330:2;
6437/chr20:3023:chr20:62463499-62463330:1;6438/chr14:704:chr14:62217978-62217804:2;6439/chr18:768:ch
r18:44526913-44526738:2;6440/chr18:768:chr18:44526913-44526738:1;6441/chr7:2591:chr7:70598008-70597
836:2;6442/chr16:757:chr16:3017515-3017363:2;6443/chr16:757:chr16:3017515-3017363:1;6444/chr7:2591:ch
r7:70598008-70597836:1;6445/chr19:566:chr19:2290728-2290568:2;6446/chr19:566:chr19:2290728-2290568:
1;6447/chr14:704:chr14:62217978-62217804:1;6448/chr7:4544:chr7:155596210-155596039:2;6449/chr7:4544:
chr7:155596210-155596039:1;6450/chr10:2698:chr10:102586948-102586807:2;6451/chr10:2698:chr10:102586
948-102586807:1;6452/chr20:2056:chr20:57766820-57766645:2;6453/chr20:2056:chr20:57766820-57766645:1;
6454/cg05920961:chr14:102248354-102248194:2;6455/cg05920961:chr14:102248354-102248194:1;6456/chr
11:2301:chr11:63767797-63767650:2;6457/chr11:2301:chr11:63767797-63767650:1;6458/chr7:2162:chr7:5034
3702-50343543:2;6459/chr7:2162:chr7:50343702-50343543:1;6460/cg23777958:chr8:85095763-85095638:2;6
461/cg23777958:chr8:85095763-85095638:1;6462/chr5:829:chr5:3595692-3595517:2;6463/chr13:1076:chr13:1
00641503-100641333:2;6464/chr6:107:chr6:1394011-1393840:2;6465/chr6:107:chr6:1394011-1393840:1;6466/
chr1:255:chr1:1567614-1567449:2;6467/chr1:255:chr1:1567614-1567449:1;6468/chr13:1076:chr13:100641503-
100641333:1;6469/chr14:322:chr14:36986901-36986740:2;6470/chr14:322:chr14:36986901-36986740:1;6471/
chr5:829:chr5:3595692-3595517:1;6472/chr20:534:chr20:21491516-21491359:2;6473/chr20:534:chr20:214915
16-21491359:1;6474/chr6:1534:chr6:41515955-41515818:2;6475/chr6:1534:chr6:41515955-41515818:1;6476/c
hr21:861:chr21:45577844-45577675:2;6477/chr21:861:chr21:45577844-45577675:1;6478/chr8:3322:chr8:1435
73119-143572953:2;6479/chr8:3322:chr8:143573119-143572953:1;6480/chr5:1812:chr5:77147970-77147806:2;
6481/chr5:1812:chr5:77147970-77147806:1;6482/chr15:2554:chr15:102030123-102029992:2;6483/chr15:2554:
chr15:102030123-102029992:1;6484/chr9:3297:chr9:139095111-139094957:2;6485/chr9:3297:chr9:13909511
1-139094957:1;6486/chr8:13:chr8:494509-494344:2;6487/chr8:13:chr8:494509-494344:1;6488/chr22:2705:chr2
2:50629383-50629218:2;6489/chr22:2705:chr22:50629383-50629218:1;6490/chr15:628:chr15:41787608-41787
467:2;6491/chr15:628:chr15:41787608-41787467:1;6492/chr1:7210:chr1:237206461-237206313:2;6493/chr1:7
210:chr1:237206461-237206313:1;6494/chr19:4915:chr19:58609563-58609438:2;6495/chr19:4915:chr19:5860
9563-58609438:1;6496/chr10:586:chr10:11060803-11060628:2;6497/chr10:586:chr10:11060803-11060628:1;6
498/chr20:1991:chr20:57224890-57224755:2;6499/chr20:1991:chr20:57224890-57224755:1;6500/chr10:3240:c
hr10:119494735-119494566:2;6501/chr10:3240:chr10:119494735-119494566:1;6502/chr2:4262:chr2:17699488
1-176994717:2;6503/chr2:4262:chr2:176994881-176994717:1;6504/chr1:3767:chr1:43250927-43250760:2;650
5/chr7:3616:chr7:130419167-130419000:2;6506/chr7:3616:chr7:130419167-130419000:1;6507/chr5:3858:chr5:
179244214-179244041:2;6508/chr5:3858:chr5:179244214-179244041:1;6509/chr20:1728:chr20:50722058-507
21930:2;6510/chr20:1728:chr20:50722058-50721930:1;6511/chr2:4127:chr2:172954892-172954727:2;6512/chr
2:4127:chr2:172954892-172954727:1;6513/chr5:3298:chr5:174159131-174159001:2;6514/chr5:3298:chr5:1741
59131-174159001:1;6515/chr2:4124:chr2:172952229-172952058:1;6516/chr22:294:chr22:20779947-20779778:
2;6517/chr18:280:chr18:11751968-11751832:2;6518/chr18:280:chr18:11751968-11751832:1;6519/chr7:3136:c
hr7:100318228-100318055:2;6520/chr7:3136:chr7:100318228-100318055:1;6521/chr3:649:chr3:22414266-224
14120:2;6522/chr3:649:chr3:22414266-22414120:1;6523/chr10:2140:chr10:81002879-81002710:2;6524/chr10:
2140:chr10:81002879-81002710:1;6525/chr22:294:chr22:20779947-20779778:1;6526/chr18:1613:chr18:76740
848-76740722:2;6527/chr18:1613:chr18:76740848-76740722:1;6528/chr4:1578:chr4:30723952-30723825:2;65
29/chr4:1578:chr4:30723952-30723825:1;6530/chr1:3953:chr1:47899693-47899545:1;6531/chr9:343:chr9:3481
0034-34809864:2;6532/chr9:343:chr9:34810034-34809864:1;6533/chr9:58:chr9:1050253-1050079:2;6534/chr9:
58:chr9:1050253-1050079:1;6535/cg07923233:chr1:76080755-76080581:1;6536/chr19:325:chr19:1302705-130
2530:1;6537/chr11:141:chr11:849413-849283:2;6538/chr11:141:chr11:849413-849283:1;6539/chr19:2725:chr1
9:31839922-31839749:2;6540/chr19:2725:chr19:31839922-31839749:1;6541/chr16:2668:chr16:58058730-5805
8572:2;6542/chr16:2668:chr16:58058730-58058572:1;6543/chr17:2375:chr17:46711576-46711403:2;6544/chr1
7:2375:chr17:46711576-46711403:1;6545/chr10:1133:chr10:35928995-35928820:2;6546/chr10:1133:chr10:359
28995-35928820:1;6547/chr4:1604:chr4:37246819-37246647:2;6548/chr4:1604:chr4:37246819-37246647:1;65
49/chr20:1381:chr20:44686224-44686050:2;6550/chr20:1381:chr20:44686224-44686050:1;6551/chr5:3133:chr
5:170878160-170878008:2;6552/chr5:3133:chr5:170878160-170878008:1;6553/chr1:3001:chr1:25255878-2525
5742:2;6554/chr1:3001:chr1:25255878-25255742:1;6555/chr2:3031:chr2:111876974-111876802:2;6556/chr11:
2488:chr11:65246454-65246279:2;6557/chr11:2488:chr11:65246454-65246279:1;6558/chr19:4904:chr19:5857
0745-58570573:2;6559/chr2:5206:chr2:236579517-236579378:2;6560/chr2:5206:chr2:236579517-236579378:1;
6561/chr17:2385:chr17:46796526-46796354:2;6562/chr17:2385:chr17:46796526-46796354:1;6563/chr7:3136:
chr7:100318493-100318348:2;6564/chr7:3136:chr7:100318493-100318348:1;6565/chr17:1813:chr17:35299612-
35299438:2;6566/chr19:367:chr19:1468463-1468290:2;6567/chr5:839:chr5:3602608-3602444:2;6568/chr5:839:
chr5:3602608-3602444:1;6569/chr16:2827:chr16:66613355-66613189:2;6570/chr16:2827:chr16:66613355-666
13189:1;6571/chr8:1738:chr8:55372241-55372068:2;6572/chr8:1738:chr8:55372241-55372068:1;6573/chr15:1
477:chr15:75248782-75248615:2;6574/chr12:428:chr12:6664866-6664692:2;6575/chr1:3867:chr1:45083235-45
083069:2;6576/chr1:3867:chr1:45083235-45083069:1;6577/chr12:1427:chr12:57619035-57618864:2;6578/chr1
2:1427:chr12:57619035-57618864:1;6579/chr12:428:chr12:6664866-6664692:1;6580/chr4:1403:chr4:13543953-
13543828:2;6581/chr4:1403:chr4:13543953-13543828:1;6582/chr2:4218:chr2:176948223-176948051:2;6583/c
hr2:4218:chr2:176948223-176948051:1;6584/chr4:1726:chr4:44449803-44449678:2;6585/chr4:1726:chr4:4444
9803-44449678:1;6586/chr16:4071:chr16:88442987-88442830:2;6587/chr10:1066:chr10:30348188-30348016:2;
6588/chr10:1066:chr10:30348188-30348016:1;6589/chr16:4071:chr16:88442987-88442830:1;6590/chr10:4269:
chr10:133109533-133109367:2;6591/chr10:4269:chr10:133109533-133109367:1;6592/chr19:4654:chr19:5559
8818-55598679:2;6593/chr19:4654:chr19:55598818-55598679:1;6594/chr4:1509:chr4:24801681-24801514:2;6
595/chr4:1509:chr4:24801681-24801514:1;6596/chr2:1903:chr2:63275975-63275807:2;6597/chr2:1903:chr2:63
275975-63275807:1;6598/chr3:138:chr3:8809868-8809739:2;6599/chr3:138:chr3:8809868-8809739:1;6600/chr
7:4109:chr7:150655406-150655279:2;6601/chr7:4109:chr7:150655406-150655279:1;6602/chr14:847:chr14:690
95125-69094951:1;6603/chr2:4191:chr2:175205271-175205110:2;6604/chr2:4191:chr2:175205271-175205110:
1;6605/cg22434409:chr4:21950872-21950708:2;6606/cg22434409:chr4:21950872-21950708:1;6607/chr10:279
5:chr10:103539232-103539067:2;6608/chr10:2795:chr10:103539232-103539067:1;6609/chr2:4269:chr2:17701
7395-177017221:2;6610/chr2:4269:chr2:177017395-177017221:1;6611/chr21:214:chr21:34442546-34442377:2;
6612/chr21:214:chr21:34442546-34442377:1;6613/chr22:2526:chr22:49969399-49969226:2;6614/chr9:3295:c
hr9:139093487-139093350:2;6615/chr9:3295:chr9:139093487-139093350:1;6616/chr4:391:chr4:2042574-2042
416:2;6617/chr4:391:chr4:2042574-2042416:1;6618/chr8:1408:chr8:38965189-38965059:2;6619/chr8:1408:chr
8:38965189-38965059:1;6620/chr17:550:chr17:4981619-4981455:1;6621/chrX:810:chrX:48684944-48684806:2;
6622/chrX:810:chrX:48684944-48684806:1;6623/chr20:516:chr20:21081964-21081794:1;6624/chr16:3863:chr
16:86962925-86962778:2;6625/chr16:3863:chr16:86962925-86962778:1;6626/chr13:260:chr13:25744959-2574
4792:2;6627/chr13:260:chr13:25744959-25744792:1;6628/chr1:1062:chr1:3310680-3310535:1;6629/chr3:2629:
chr3:138658626-138658498:2;6630/chr3:2629:chr3:138658626-138658498:1;6631/chr17:4139:chr17:77789414-
77789287:2;6632/chr17:4139:chr17:77789414-77789287:1;6633/chr13:1700:chr13:113764161-113763987:2;6
634/chr13:1700:chr13:113764161-113763987:1;6635/chr1:2371:chr1:16085950-16085815:2;6636/chr1:2371:ch
r1:16085950-16085815:1;6637/chr3:1010:chr3:45187917-45187789:2;6638/chr3:1010:chr3:45187917-4518778
9:1;6639/chr10:1066:chr10:30347877-30347702:2;6640/chr10:1066:chr10:30347877-30347702:1;6641/chr11:4
166:chr11:119211606-119211474:2;6642/chr11:4166:chr11:119211606-119211474:1;6643/chr4:1280:chr4:887
4635-8874505:2;6644/chr4:1280:chr4:8874635-8874505:1;6645/chr22:2526:chr22:49969399-49969226:1;6646/
chr10:2022:chr10:77156283-77156157:2;6647/chr10:2022:chr10:77156283-77156157:1;6648/chr1:1408:chr1:4
7145504714421:2;6649/chr1:1408:chr1:4714550-4714421:1;6650/chr6:1872:chr6:72892544-72892408:2;6651/
chr6:1872:chr6:72892544-72892408:1;6652/chr2:4572:chr2:209271606-209271469:2;6653/chr2:4572:chr2:209
271606-209271469:1;6654/chr10:4732:chr10:134844974-134844820:2;6655/chr10:4732:chr10:134844974-134
844820:1;6656/chr17:2173:chr17:43037339-43037212:2;6657/chr17:2173:chr17:43037339-43037212:1;6658/c
hr11:3337:chr11:72295824-72295699:2;6659/chr11:3337:chr11:72295824-72295699:1;6660/chr22:141:chr22:1
9512253-19512078:1;6661/chrX:504:chrX:21676692-21676518:1;6662/chr14:1032:chr14:75078523-75078378:
2;6663/chr14:1032:chr14:75078523-75078378:1;6664/chr5:1055:chr5:7850347-7850212:2;6665/chr5:1055:chr5:
7850347-7850212:1;6666/chr3:3077:chr3:181413442-181413268:2;6667/chr3:3077:chr3:181413442-18141326
8:1;6668/chr11:3337:chr11:72295574-72295401:2;6669/chr11:3337:chr11:72295574-72295401:1;6670/chr10:4
051:chr10:131757398-131757227:2;6671/chr10:4051:chr10:131757398-131757227:1;6672/chr5:1710:chr5:725
94527-72594392:2;6673/chr5:1710:chr5:72594527-72594392:1;6674/chr6:1567:chr6:4207217642072009:2;66
75/chr6:1567:chr6:42072176-42072009:1;6676/chr2:3031:chr2:111876974-111876802:1;6677/chr19:4904:chr1
9:58570745-58570573:1;6678/chr3:823:chr3:37494227-37494060:2;6679/chr3:823:chr3:37494227-37494060:1;
6680/chr16:756:chr16:3017149-3016977:2;6681/chr16:756:chr16:3017149-3016977:1;6682/chr17:1813:chr17:3
5299612-35299438:1;6683/chr19:367:chr19:1468463-1468290:1;6684/chr7:2143:chr7:4981328149813110:2;6
685/chr7:2143:chr7:49813281-49813110:1;6686/chr15:1477:chr15:75248782-75248615:1;6687/chr1:2566:chr1:
18434656-18434531:2;6688/chr1:2566:chr1:18434656-18434531:1;6689/chr21:948:chr21:45875778-45875652:
2;6690/chr21:948:chr21:45875778-45875652:1;6691/chr2:3075:chr2:114035498-114035371:2;6692/chr2:3075:
chr2:114035498-114035371:1;6693/chr1:4363:chr1:63785498-63785324:2;6694/chr1:4363:chr1:63785498-637
85324:1;6695/chr16:112:chr16:745166-745023:2;6696/chr16:112:chr16:745166-745023:1;6697/chr13:386:chr1
3:29107292-29107120:2;6698/chr13:386:chr13:29107292-29107120:1;6699/chr8:809:chr8:17270919-17270791:
2;6700/chr8:809:chr8:17270919-17270791:1;6701/chr1:4838:chr1:110754788-110754619:2;6702/chr1:4838:ch
r1:110754788-110754619:1;6703/chr5:511:chr5:1886462-1886336:1;6704/chr4:1265:chr4:8860121-8859985:2;
6705/chr4:1265:chr4:8860121-8859985:1;6706/chr7:547:chr7:1577317-1577190:2;6707/chr7:547:chr7:1577317-
1577190:1;6708/chr18:105:chr18:5890681-5890541:2;6709/chr18:105:chr18:5890681-5890541:1;6710/chr7:18
58:chr7:37487700-37487532:2;6711/chr7:1858:chr7:37487700-37487532:1;6712/cg25299895:chr13:28498161-
28497986:1;6713/chr19:3890:chr19:4715331147153136:2;6714/chr19:3890:chr19:4715331147153136:1;6715/
chr5:1718:chr5:72677488-72677363:2;6716/chr5:1718:chr5:72677488-72677363:1;6717/chr5:221:chr5:105933
0-1059156:1;6718/chr5:705:chr5:2751652-2751524:2;6719/chr5:705:chr5:2751652-2751524:1;6720/chr14:2327:
chr14:104622118-104621990:2;6721/chr14:2327:chr14:104622118-104621990:1;6722/chr22:1851:chr22:4647
4687-46474561:2;6723/chr22:1851:chr22:46474687-46474561:1;6724/chr16:241:chr16:1041296-1041165:2;67
25/chr16:241:chr16:1041296-1041165:1;6726/chr2:3710:chr2:131721897-131721724:2;6727/chr2:3710:chr2:13
1721897-131721724:1;6728/chr2:4772:chr2:220313817-220313646:2;6729/chr2:4772:chr2:220313817-220313
646:1;6730/chrX:810:chrX:48685294-48685152:2;6731/chrX:810:chrX:48685294-48685152:1;6732/chr15:207:c
hr15:27112220-27112083:2;6733/chr15:207:chr15:27112220-27112083:1;6734/chr18:1054:chr18:57364452-57
364277:2;6735/chr19:2193:chr19:18771514-18771374:1;6736/chr8:3322:chr8:143573022-143572886:2;6737/c
hr8:3322:chr8:143573022-143572886:1;6738/chr18:1054:chr18:57364452-57364277:1;6739/chr15:937:chr15:5
8358141-58358016:2;6740/chr15:937:chr15:58358141-58358016:1;6741/chr17:2190:chr17:43198720-4319855
0:2;6742/chr17:2190:chr17:4319872043198550:1;6743/chr7:2143:chr7:4981333749813175:2;6744/chr7:2143:
chr7:49813337-49813175:1;6745/chr8:3702:chr8:145105505-145105375:1;6746/chr11:5:chr11:280314-280143:
2;6747/chr11:5:chr11:280314-280143:1;6748/chr20:1812:chr20:55202207-55202065:2;6749/chr20:1812:chr20:
55202207-55202065:1;6750/chr6:317:chr6:7051823-7051655:2;6751/chr6:317:chr6:7051823-7051655:1;6752/c
hr7:4565:chr7:155744407-155744232:1;6753/cg04034767:chr12:52400977-52400841:2;6754/cg04034767:chr1
2:52400977-52400841:1;6755/chr13:699:chr13:53174467-53174336:2;6756/chr13:699:chr13:53174467-531743
36:1;6757/chr11:2774:chr11:68145602-68145477:1;6758/chr16:1124:chr16:9107382-9107231:2;6759/chr16:11
24:chr16:9107382-9107231:1;6760/chr1:3767:chr1:43250927-43250760:1;6761/chr15:1832:chr15:83953184-83
953010:2;6762/chr15:1832:chr15:83953184-83953010:1;6763/chr10:3443:chr10:124894939-124894769:2;6764/
chr10:3443:chr10:124894939-124894769:1;6765/chr12:428:chr12:6665303-6665165:2;6766/chr12:428:chr12:6
665303-6665165:1;6767/chr1:7209:chr1:237205587-237205414:2;6768/chr1:7209:chr1:237205587-237205414:
1;6769/chr3:2172:chr3:124860761-124860615:2;6770/chr3:2172:chr3:124860761-124860615:1;6771/chr17:253
7:chr17:48546777-48546609:2;6772/chr17:2537:chr17:48546777-48546609:1;6773/chr17:550:chr17:4981769-4
981595:2;6774/chr17:550:chr17:4981769-4981595:1;6775/chr2:4251:chr2:176987309-176987147:2;6776/chr2:
4251:chr2:176987309-176987147:1;6777/chr1:3370:chr1:32827550-32827380:2;6778/chr1:3370:chr1:3282755
0-32827380:1;6779/chr3:2753:chr3:147111704-147111529:2;6780/chr3:2753:chr3:147111704-147111529:1;67
81/chr2:4287:chr2:177043176-177043007:2;6782/chr2:4287:chr2:177043176-177043007:1;6783/chr14:2758:ch
r14:105944557-105944401:2;6784/chr14:2758:chr14:105944557-105944401:1;6785/chr12:3126:chr12:1309092
20-130909058:2;6786/chr12:3126:chr12:130909220-130909058:1;6787/chr15:1558:chr15:76639697-76639572:
2;6788/chr15:1558:chr15:76639697-76639572:1;6789/chr9:3751:chr9:140681418-140681249:1;6790/chr12:146
2:chr12:58131446-58131273:2;6791/chr12:1462:chr12:58131446-58131273:1;6792/chr10:3746:chr10:1295373
18-129537167:2;6793/chr10:3746:chr10:129537318-129537167:1;6794/chr9:47:chr9:976857-976684:2;6795/ch
r9:47:chr9:976857-976684:1;6796/chr10:4582:chr10:134598798-134598673:2;6797/chr10:4582:chr10:1345987
98-134598673:1;6798/chr14:157:chr14:24780604-24780434:2;6799/chr14:157:chr14:24780604-24780434:1;68
00/chr6:1630:chr6:43252901-43252734:2;6801/chr6:1630:chr6:43252901-43252734:1;6802/chr10:1956:chr10:7
3847266-73847140:2;6803/chr19:158:chr19:863185-863010:2;6804/chr19:158:chr19:863185-863010:1;6805/ch
r12:1090:chr12:50355414-50355286:2;6806/chr12:1090:chr12:50355414-50355286:1;6807/chr10:1956:chr10:7
3847266-73847140:1;6808/chr4:1403:chr4:13543849-13543707:1;6809/chr14:625:chr14:59931204-59931061:1;
6810/chr11:2620:chr11:66886080-66885906:2;6811/chr11:2620:chr11:66886080-66885906:1;6812/chr13:657:
chr13:49795530-49795359:2;6813/chr13:657:chr13:49795530-49795359:1;6814/chr8:2423:chr8:120220830-12
0220670:2;6815/chr8:2423:chr8:120220830-120220670:1;6816/chr9:2923:chr9:137967528-137967353:2;6817/
chr19:2540:chr19:30017269-30017097:2;6818/chr19:2540:chr19:30017269-30017097:1;6819/chr9:2923:chr9:1
37967528-137967353:1;6820/chr17:715:chr17:7287370-7287196:2;6821/chr17:715:chr17:7287370-7287196:1;
6822/chr19:4357:chr19:52223159-52222988:2;6823/chr19:4357:chr19:52223159-52222988:1;6824/chr1:3659:c
hr1:39957684-39957540:2;6825/chr1:3659:chr1:39957684-39957540:1;6826/chr5:1664:chr5:66462327-664621
55:1;6827/chr18:105:chr18:5890548-5890382:2;6828/chr18:105:chr18:5890548-5890382:1;6829/chr10:3506:ch
r10:125651454-125651279:2;6830/chr10:3506:chr10:125651454-125651279:1;6831/chr20:1810:chr20:5520149
5-55201349:2;6832/chr15:1205:chr15:68122515-68122343:2;6833/chr15:1205:chr15:68122515-68122343:1;68
34/chr3:1611:chr3:65342300-65342125:2;6835/chr3:1611:chr3:65342300-65342125:1;6836/chr20:1810:chr20:5
5201495-55201349:1;6837/chr3:1158:chr3:48699153-48698986:2;6838/chr3:1158:chr3:48699153-48698986:1;
6839/chr10:4752:chr10:134862637-134862480:1;6840/chr16:2379:chr16:51168611-51168469:2;6841/chr16:23
79:chr16:51168611-51168469:1;6842/chr1:135:chr1:1118671-1118528:2;6843/chr1:135:chr1:1118671-1118528:
1;6844/chr19:1881:chr19:15580815-15580649:2;6845/chr19:1881:chr19:15580815-15580649:1;6846/chr2:428
4:chr2:177036815-177036682:1;6847/chr1:4364:chr1:63785814-63785670:1;6848/chr17:965:chr17:10102363-1
0102224:2;6849/chr17:965:chr17:10102363-10102224:1;6850/chr4:1604:chr4:37246898-37246733:2;6851/chr1:
4640:chr1:91301852-91301681:2;6852/chr1:4640:chr1:91301852-91301681:1;6853/chr1:971:chr1:3155292-31
55119:2;6854/chr1:971:chr1:3155292-3155119:1;6855/chr2:598:chr2:5832671-5832497:2;6856/chr2:598:chr2:5
832671-5832497:1;6857/chr2:4269:chr2:177017327-177017154:2;6858/chr22:1790:chr22:46263196-46263057:
2;6859/chr7:1788:chr7:35293784-35293621:2;6860/chr7:1788:chr7:35293784-35293621:1;6861/chr5:1679:chr5:
68711295-68711120:2;6862/chr5:1679:chr5:68711295-68711120:1;6863/chr22:1873:chr22:46500451-4650028
1:2;6864/chr22:1873:chr22:46500451-46500281:1;6865/chr17:2349:chr17:46604695-46604541:2;6866/chr17:2
349:chr17:46604695-46604541:1;6867/chr11:1212:chr11:13031032-13030857:2;6868/chr11:1212:chr11:13031
032-13030857:1;6869/chr5:3695:chr5:178422228-178422056:2;6870/chr5:3695:chr5:178422228-178422056:1;
6871/chr14:2833:chr14:106320581-106320455:2;6872/chr14:2833:chr14:106320581-106320455:1;6873/chr19:
3840:chr19:46518586-46518446:2;6874/chr19:3840:chr19:46518586-46518446:1;6875/chr2:5245:chr2:237082
107-237081962:1;6876/chr1:254:chr1:1566795-1566623:1;6877/chr22:2722:chr22:50706465-50706291:2;6878/
chr22:2722:chr22:50706465-50706291:1;6879/chr4:759:chr4:5710322-5710190:2;6880/chr4:759:chr4:5710322-
5710190:1;6881/chr12:1460:chr12:58130055-58129888:2;6882/chr12:1460:chr12:58130055-58129888:1;6883/
chr8:33:chr8:638251-638120:2;6884/chr8:33:chr8:638251-638120:1;6885/chr4:1604:chr4:37246898-37246733:
1;6886/chr2:4269:chr2:177017327-177017154:1;6887/chr22:1790:chr22:46263196-46263057:1;6888/chr20:90:
chr20:1784635-1784508:2;6889/chr20:90:chr20:1784635-1784508:1;6890/chr5:1664:chr5:66462378-66462241:
2;6891/chr5:1664:chr5:66462378-66462241:1;6892/chr6:21:chr6:391986-391859:2;6893/chr6:21:chr6:391986-3
91859:1;6894/chr7:562:chr7:1626297-1626123:1;6895/chr11:2492:chr11:65314648-65314482:1;6896/chr11:36
63:chr11:94134617-94134444:1;6897/cg23142394:chr12:72665842-72665678:1;6898/chr10:3131:chr10:11803
1131-118030959:2;6899/chr10:3131:chr10:118031131-118030959:1;6900/chr2:1519:chr2:39187616-39187481:
2;6901/chr2:1519:chr2:39187616-39187481:1;6902/chr21:59:chr21:19617319-19617176:2;6903/chr21:59:chr21:
19617319-19617176:1;6904/chr12:1757:chr12:95942130-95941961:2;6905/chr12:1757:chr12:95942130-95941
961:1;6906/chr19:322:chr19:1295797-1295624:2;6907/chr19:322:chr19:1295797-1295624:1;6908/chr19:1696:c
hr19:13210007-13209854:2;6909/chr19:1696:chr19:13210007-13209854:1;6910/chr3:2880:chr3:156544442-15
6544312:2;6911/chr16:4191:chr16:88701359-88701234:2;6912/chr16:4191:chr16:88701359-88701234:1;6913/
chr3:2880:chr3:156544442-156544312:1;6914/chr6:279:chr6:6004447-6004321:2;6915/chr6:279:chr6:6004447-
6004321:1;6916/chr17:279:chr17:1962404-1962231:2;6917/chr17:279:chr17:1962404-1962231:1;6918/chr3:10
10:chr3:45188092-45187928:2;6919/chr3:1010:chr3:45188092-45187928:1;6920/chr20:2677:chr20:61638258-6
1638084:2;6921/chr20:2677:chr20:61638258-61638084:1;6922/chr8:565:chr8:8560013-8559838:2;6923/chr8:5
65:chr8:8560013-8559838:1;6924/chr13:368:chr13:28674285-28674159:2;6925/chr13:368:chr13:28674285-286
74159:1;6926/chr2:5240:chr2:237078584-237078417:1;6927/chr20:1381:chr20:44686146-44685971:2;6928/chr
20:1381:chr20:44686146-44685971:1;6929/chr2:4288:chr2:177053961-177053792:2;6930/chr2:4288:chr2:1770
53961-177053792:1;6931/chr10:3456:chr10:124909280-124909106:2;6932/chr10:3456:chr10:124909280-1249
09106:1;6933/chr22:1865:chr22:46484982-46484808:1;6934/chr8:42:chr8:689086-688916:2;6935/chr8:42:chr8:
689086-688916:1;6936/chr10:2309:chr10:88123490-88123361:2;6937/chr10:2309:chr10:88123490-88123361:1;
6938/chr12:1858:chr12:104610007-104609837:2;6939/chr12:1858:chr12:104610007-104609837:1;6940/chr10:
1587:chr10:52177955-52177792:2;6941/chr10:1587:chr10:52177955-52177792:1;6942/chr18:1804:chr18:7738
9526-77389351:2;6943/chr18:1804:chr18:77389526-77389351:1;6944/cg24371075:chr17:8533483-8533318:1;
6945/chr12:2900:chr12:127630688-127630531:2;6946/chr12:2900:chr12:127630688-127630531:1;6947/chr19:
4075:chr19:49575789-49575636:2;6948/chr19:4075:chr19:49575789-49575636:1;6949/chr7:4816:chr7:157477
806-157477638:2;6950/chr7:4816:chr7:157477806-157477638:1;6951/chr4:1265:chr4:8860228-8860099:2;695
2/chr4:1265:chr4:8860228-8860099:1;6953/chr6:596:chr6:19837840-19837707:2;6954/chr6:596:chr6:19837840-
19837707:1;6955/chr14:1435:chr14:93154672-93154497:2;6956/chr14:1435:chr14:93154672-93154497:1;695
7/chr4:1509:chr4:24801918-24801782:2;6958/chr4:1509:chr4:24801918-24801782:1;6959/chr10:524:chr10:809
7648-8097507:2;6960/chr10:524:chr10:8097648-8097507:1;6961/chr18:92:chr18:5543635-5543510:2;6962/chr
18:92:chr18:5543635-5543510:1;6963/chr4:241:chr4:1537856-1537687:2;6964/chr4:241:chr4:1537856-153768
7:1;6965/chr12:1169:chr12:52627282-52627115:2;6966/chr12:1169:chr12:52627282-52627115:1;6967/chr7:43
03:chr7:152623133-152622977:2;6968/chr7:4303:chr7:152623133-152622977:1;6969/chr20:2210:chr20:59827
381-59827242:2;6970/chr20:2210:chr20:59827381-59827242:1;6971/chr11:461:chr11:1864088-1863913:1;697
2/chr16:2474:chr16:54970577-54970429:2;6973/chr16:2474:chr16:54970577-54970429:1;6974/chr1:4363:chr1:
63785715-63785590:2;6975/chr1:4363:chr1:63785715-63785590:1;6976/chr8:96:chr8:876975-876850:2;6977/c
hr8:96:chr8:876975-876850:1;6978/chr22:163:chr22:19712189-19712031:2;6979/chr22:163:chr22:19712189-1
9712031:1;6980/chr19:4944:chr19:58907831-58907705:2;6981/chr19:4944:chr19:58907831-58907705:1;6982/
chr12:1090:chr12:50354909-50354776:2;6983/chr12:1090:chr12:50354909-50354776:1;6984/chr1:4051:chr1:5
0887227-50887061:2;6985/chr1:4051:chr1:50887227-50887061:1;6986/chr19:2639:chr19:30866417-30866285:
2;6987/chr19:2639:chr19:30866417-30866285:1;6988/chr19:1986:chr19:17008587-17008422:2;6989/chr19:198
6:chr19:17008587-17008422:1;6990/chr17:534:chr17:4803076-4802951:2;6991/chr17:534:chr17:4803076-4802
951:1;6992/chr1:4363:chr1:63785419-63785248:2;6993/chr1:4363:chr1:63785419-63785248:1;6994/chr10:274
0:chr10:102900085-102899927:2;6995/chr10:2740:chr10:102900085-102899927:1;6996/chr12:1427:chr12:576
18793-57618624:2;6997/chr12:1427:chr12:57618793-57618624:1;6998/chr14:2764:chr14:105953781-1059536
22:2;6999/chr14:2764:chr14:105953781-105953622:1;7000/chr2:1990:chr2:66672796-66672671:2;7001/chr2:1
990:chr2:66672796-66672671:1;7002/chr3:2383:chr3:128211165-128211020:2;7003/chr3:2383:chr3:12821116
5-128211020:1;7004/chr15:1832:chr15:83953816-83953648:2;7005/chr18:610:chr18:34833804-34833645:2;70
06/chr18:610:chr18:34833804-34833645:1;7007/chr12:1171:chr12:52652435-52652289:2;7008/chr12:1171:chr
12:52652435-52652289:1;7009/chr5:2253:chr5:132158758-132158611:2;7010/chr5:2253:chr5:132158758-1321
58611:1;7011/chr11:98:chr11:627097-626964:2;7012/chr11:98:chr11:627097-626964:1;7013/chr2:599:chr2:583
3018-5832848:2:7014/chr2:599:chr2:5833018-5832848:1;7015/chr20:525:chr20:21377389-21377222:2;7016/ch
r20:525:chr20:21377389-21377222:1;7017/chr10:2424:chr10:94822729-94822566:2;7018/chr10:2424:chr10:94
822729-94822566:1;7019/chr4:1747:chr4:48485336-48485192:2;7020/chr4:1747:chr4:48485336-48485192:1;7
021/eg16302790:chr13:28498478-28498312:1;7022/cg02914087:chr19:12267802-12267629:1;7023/chr4:1265:
chr4:8860496-8860352:2;7024/chr4:1265:chr4:8860496-8860352:1;7025/chr19:4357:chr19:52223231-5222307
2:1;7026/chr6:2249:chr6:108439156-108439028:2;7027/chr6:2249:chr6:108439156-108439028:1;7028/chr1:48
61:chr1:111217129-111216966:2;7029/chr15:1832:chr15:83953816-83953648:1;7030/chr1:4861:chr1:1112171
29-111216966:1;7031/chr17:859:chr17:8926822-8926658:2;7032/chr17:859:chr17:8926822-8926658:1;7033/ch
r13:289:chr13:26625717-26625560:2;7034/chr13:289:chr13:26625717-26625560:1;7035/chr17:534:chr17:4802
879-4802720:2;7036/chr17:534:chr17:4802879-4802720:1;7037/chr1:691:chr1:2777435-2777274:1;7038/chr7:3
586:chr7:129421964-129421839:2;7039/chr1:4566:chr1:86622652-86622486:1;7040/chr17:1003:chr17:111442
90-11144155:2;7041/chr17:1003:chr17:11144290-11144155:1;7042/chr19:759:chr19:3435387-3435231:2;7043/
chr19:759:chr19:3435387-3435231:1;7044/chr7:3586:chr7:129421964-129421839:1;7045/chr5:827:chr5:35947
99-3594626:2;7046/chr5:827:chr5:3594799-3594626:1;7047/chr1:3957:chr1:47905040-47904908:2;7048/chr1:3
957:chr1:47905040-47904908:1;7049/chr3:333:chr3:13115024-13114899:2;7050/chr3:333:chr3:13115024-1311
4899:1;7051/chr7:1887:chr7:38670916-38670742:2;7052/chr7:1887:chr7:38670916-38670742:1;7053/chr2:418
3:chr2:175200801-175200635:2;7054/chr2:4183:chr2:175200801-175200635:1;7055/chr18:566:chr18:3102055
6-31020413:2;7056/chr18:566:chr18:31020556-31020413:1;7057/chr20:2212:chr20:59828696-59828571:2;705
8/chr20:2212:chr20:59828696-59828571:1;7059/chr14:986:chr14:73958336-73958174:2;7060/chr14:986:chr14:
73958336-73958174:1;7061/chr9:135:chr9:8857751-8857626:2;7062/chr9:135:chr9:8857751-8857626:1;7063/c
hr6:279:chr6:6004341-6004181:2;7064/chr6:279:chr6:6004341-6004181:1;7065/chr13:85:chr13:20876202-208
76036:2;7066/chr13:85:chr13:20876202-20876036:1;7067/chr8:1971:chr8:72460393-72460220:2;7068/chr8:19
71:chr8:72460393-72460220:1;7069/chr14:688:chr14:61747073-61746948:2;7070/chr14:688:chr14:61747073-6
1746948:1;7071/chr10:4583:chr10:134599342-134599181:2;7072/chr10:4583:chr10:134599342-134599181:1;7
073/chr5:2325:chr5:134364461-134364301:2;7074/chr5:2325:chr5:134364461-134364301:1;7075/chr17:275:ch
r17:1960642-1960505:2;7076/chr17:275:chr17:1960642-1960505:1;7077/chr8:38:chr8:687491-687318:2;7078/c
hr8:38:chr8:687491-687318:1;7079/chr6:1972:chr6:84419038-84418886:2;7080/chr6:1972:chr6:84419038-844
18886:1;7081/chr17:2799:chr17:59474611-59474474:2;7082/chr17:2799:chr17:59474611-59474474:1;7083/chr
1:1739:chr1:7022201-7022032:2;7084/chr1:1739:chr1:7022201-7022032:1;7085/chr6:3358:chr6:168502135-16
8502006:2;7086/chr6:3358:chr6:168502135-168502006:1;7087/chr18:585:chr18:32847415-32847277:2;7088/c
hr18:585:chr18:32847415-32847277:1;7089/chr2:1181:chr2:24232849-24232692:2;7090/chr2:1181:chr2:24232
849-24232692:1;7091/chr7:282:chr7:751835-751668:2;7092/chr7:282:chr7:751835-751668:1;7093/chr4:1405:c
hr4:13545975-13545813:2;7094/chr4:1405:chr4:13545975-13545813:1;7095/chr15:628:chr15:41787903-41787
736:1;7096/chr21:1265:chr21:47062703-47062554:2;7097/chr21:1265:chr21:47062703-47062554:1;7098/chr10:
4582:chr10:134598866-134598703:2;7099/chr10:4582:chr10:134598866-134598703:1;7100/chr6:2190:chr6:10
5389528-105389355:2;7101/chr6:2190:chr6:105389528-105389355:1;7102/chr9:1070:chr9:96714098-9671393
7:2;7103/chr9:1070:chr9:96714098-96713937:1;7104/chr7:1788:chr7:35294102-35293946:2;7105/chr7:1788:ch
r7:35294102-35293946:1;7106/cg24185576:chr11:30607499-30607360:2;7107/cg24185576:chr11:30607499-3
0607360:1;7108/chr6:1002:chr6:31466255-31466101:2;7109/chr6:1002:chr6:31466255-31466101:1

TABLE 4
chr1:762994-763035/OR4F16 (−140981), SAMD11 (−98106); chr1:859962-859978/SAMD11 (−1151); chr1:89521
3-895398/KLHL17 (−661), NOC2L (−627); chr1:932385-932531/HES4 (+3094), KLHL17 (+36491); chr1:933552-
933904/HES4 (+1824), KLHL17 (+37761); chr1:955941-955967/AGRN (+451); chr1:968387-968443/AGRN (+1
2912), RNF223 (+41272); chr1:968885-968911/AGRN (+13395), RNF223 (+40789); chr1:969239-969501/AGR
N (+13867), RNF223 (+40317); chr1:969618-969692/AGRN (+14152), RNF223 (+40032); chr1:975520-975561/
AGRN (+20038), RNF223 (+34146); chr1:975943-975968/AGRN (+20453), RNF223 (+33731); chr1:976069-97
6090/AGRN (+20577), RNF223 (+33607); chr1:1015298-1015348/RNF223 (−5636), C1orf159 (+36413); chr1:10
51331-1051363/C1orf159 (+389); chr1:1118474-1118533/TTLL10 (+9218), TNFRSF18 (+23585); chr1:1118729-
1118849/TTLL10 (+9503), TNFRSF18 (+23300); chr1:1148296-1148327/TNFRSF18 (−6223), TNFRSF4 (+123
6); chr1:1149103-1149146/TNFRSF4 (+423); chr1:1242645-1242698/PUSL1 (−1322), ACAP3 (+597); chr1:1260
013-1260039/GLTPD1 (−117), CPSF3L (+20); chr1:1260434-1260490/CPSF3L (−416), GLTPD1 (+319); chr1:12
90444-1290466/DVL1 (−5963), MXRA8 (+3460); chr1:1335160-1335207/CCNL2 (−466); chr1:1362229-1362339/
TMEM88B (+776); chr1:1362978-1362991/VWA1 (−7918), TMEM88B (+1477); chr1:1369615-1369678/VWA1
(−1256); chr1:1475085-1475208/TMEM240 (+593); chr1:1475940-1476114/TMEM240 (−287); chr1:1510494-1510
559/SSU72 (−265); chr1:1534767-1534828/SSU72 (−24536), MIB2 (−15997); chr1:1564532-1564676/MMP23B
(−2956); chr1:1564793-1564890/MMP23B (−2718); chr1:1566136-1566482/MMP23B (−1251); chr1:1566652-1566
698/MMP23B (−885); chr1:1590406-1590458/MMP23B (+22872), SLC35E2B (+33811); chr1:1590669-1590738/
MMP23B (+23144), SLC35E2B (+33539); chr1:1714005-1714067/NADK (−2528); chr1:1822233-1822263/GNB1
(+278); chr1:1822457-1822503/GNB1 (+46); chr1:1830580-1830692/CALML6 (−15630), GNB1 (−8110); chr1:18
50862-1850928/TMEM52 (−155); chr1:1935207-1935231/TMEM52 (−84479), GABRD (−15549); chr1:2080330-2
080382/SKI (−79778), PRKCZ (+98447); chr1:2137205-2137236/SKI (−22913), PRKCZ (+155312); chr1:214479
8-2144854/SKI (−15308), PRKCZ (+162917); chr1:2232143-2232194/RER1 (−91045), SKI (+72035); chr1:22323
29-2232390/RER1 (−90854), SKI (+72226); chr1:2246101-2246227/RER1 (−77050), SKI (+86030); chr1:234727
5-2347344/PEX10 (−3300); chr1:2460496-2460580/PANK4 (−2503); chr1:2705906-2706307/TTC34 (+123); chr1:
2706452-2706722/TTC34 (−357); chr1:2773473-2773565/ACTRT2 (−164527), TTC34 (−67289); chr1:2804568-2
804672/ACTRT2 (−133426), TTC34 (−98390); chr1:2805351-2805482/ACTRT2 (−132629), TTC34 (−99187); chr1:
2889963-2890046/TTC34 (−183775), ACTRT2 (−48041); chr1:2929018-2929064/TTC34 (−222811), ACTRT2 (−9
005); chr1:2987562-2987628/ARHGEF16 (−383552), PRDM16 (+1853); chr1:2987741-2987819/ARHGEF16 (−3
83367), PRDM16 (+2038); chr1:2989596-2989682/ARHGEF16 (−381508), PRDM16 (+3897); chr1:2990108-299
0150/ARHGEF16 (−381018), PRDM16 (+4387); chr1:2990262-2990371/ARHGEF16 (−380830), PRDM16 (+457
5); chr1:3013166-3013246/ARHGEF16 (−357941), PRDM16 (+27464); chr1:3077622-3077834/ARHGEF16 (−29
3419), PRDM16 (+91986); chr1:3164348-3164430/ARHGEF16 (−206758), PRDM16 (+178647); chr1:3310286-3
310316/ARHGEF16 (−60846), PRDM16 (+324559); chr1:3310671-3310755/ARHGEF16 (−60434), PRDM16 (+3
24971); chr1:3454818-3454857/MEGF6 (+73221), ARHGEF16 (+83691); chr1:3513849-3513883/MEGF6 (+141
93), ARHGEF16 (+142719); chr1:3528416-3528466/MEGF6 (−382); chr1:3528679-3528749/MEGF6 (−655); chr
1:3540767-3540810/TPRG1L (−767); chr1:3566445-3566487/TP73 (−2663), WRAP73 (+205); chr1:3567345-356
7572/TP73 (−1670), WRAP73 (−788); chr1:3567851-3567882/TP73 (−1262), WRAP73 (−1196); chr1:3595011-35
95134/LOC388588 (−94279), TP73 (+25944); chr1:3623318-3623338/LOC388588 (−66024), TP73 (+54199); chr
1:3659643-3659671/LOC388588 (−29695), TP73 (+90528); chr1:4714075-4714163/AJAP1 (−986); chr1:471436
5-4714488/AJAP1 (−678); chr1:4714973-4714990/AJAP1 (−123); chr1:4715120-4715192/AJAP1 (+51); chr1:471
5540-4715574/AJAP1 (+452); chr1:5819215-5819307/NPHP4 (+233272); chr1:5896886-5896970/NPHP4 (+155
605); chr1:6053562-6053624/NPHP4 (−1060); chr1:6054807-6054916/NPHP4 (−2329); chr1:6086714-6086839/
NPHP4 (−34244), KCNAB2 (−19204); chr1:6231012-6231088/CHD5 (+9144), KCNAB2 (+125069); chr1:6295552-
6295610/ICMT (+463); chr1:6305736-6305783/HES3 (+1508), GPR153 (+15275); chr1:6306075-6306125/HES
3 (+1848), GPR153 (+14935); chr1:6306305-6306351/HES3 (+2076), GPR153 (+14707); chr1:6306574-630661
3/HES3 (+2342), GPR153 (+14441); chr1:6500988-6501054/ESPN (+16173), TNFRSF25 (+25234); chr1:65147
48-6514803/TNFRSF25 (+11479), ESPN (+29928); chr1:6515011-6515220/TNFRSF25 (+11139), ESPN (+302
68); chr1:6515623-6515713/TNFRSF25 (+10587), ESPN (+30820); chr1:6515884-6515944/TNFRSF25 (+1034
1), ESPN (+31066); chr1:6520633-6520682/TNFRSF25 (+5597), ESPN (+35810); chr1:6526054-6526181/TNF
RSF25 (+137); chr1:6545194-6545271/TNFRSF25 (−18978), PLEKHG5 (+12251); chr1:6569575-6569604/PLE
KHG5 (−12106), NOL9 (+45068); chr1:6640291-6640359/ZBTB48 (+262); chr1:6640942-6641091/ZBTB48 (+95
4); chr1:6663681-6663729/KLHL21 (−776); chr1:6672767-6672834/PHF13 (−955); chr1:6846220-6846273/CAM
TA1 (+863); chr1:7022107-7022260/VAMP3 (−809145), CAMTA1 (+176800); chr1:7311136-7311191/VAMP3
(−520165), CAMTA1 (+465780); chr1:7705516-7705676/VAMP3 (−125733), CAMTA1 (+860212); chr1:8214951-8
214962/SLC45A1 (−169433), ERRFI1 (−128564); chr1:8938599-8938656/ENO1 (+523); chr1:9188906-9188943/
GPR157 (+304); chr1:9189668-9189741/GPR157 (−476); chr1:9295012-9295050/H6PD (+168); chr1:9380768-9
380799/SLC25A33 (−218744), SPSB1 (+27843); chr1:9648776-9648796/TMEM201 (−146); chr1:9883572-98836
61/CLSTN1 (+933); chr1:9884358-9884395/CLSTN1 (+173); chr1:10698898-10698908/CASZ1 (+157804), PEX
14 (+163900); chr1:10776484-10776650/CASZ1 (+80140), PEX14 (+241564); chr1:10948713-10948746/TARD
BP (−123949), CASZ1 (−92023); chr1:11539996-11540039/PTCHD2 (+723); chr1:11540179-11540380/PTCHD2
(+985); chr1:11751815-11751903/MAD2L2 (−181), C1orf187 (+78); chr1:11752161-11752230/MAD2L2 (−518),
C1orf187 (+415); chr1:11841615-11841704/MTHFR (+24500), AGTRAP (+45518); chr1:11968137-11968197/N
PPB (−49175), KIAA2013 (+18318); chr1:12079233-12079273/TNFRSF8 (−44181), MFN2 (+39015); chr1:12656
131-12656174/DHRS3 (+21667), VPS13D (+366040); chr1:13839769-13840077/LRRC38 (+319); chr1:1391024
8-13910310/PDPN (+27); chr1:13910757-13910793/PDPN (+523); chr1:15438947-15439012/TMEM51 (−41249),
KAZN (+513767); chr1:15510330-15510412/EFHD2 (−226020), TMEM51 (+30142); chr1:15672511-15672605/
EFHD2 (−63833), TMEM51 (+192329); chr1:15943864-15943882/DDI2 (−80); chr1:16084772-16084839/FBLIM1
(−449); chr1:16091589-16091633/FBLIM1 (+6356), UQCRHL (+42583); chr1:16301902-16301938/ZBTB17 (+7
07); chr1:16390527-16390626/CLCNKA (+42091), EPHA2 (+92005); chr1:16489198-16489265/EPHA2 (−6650),
ARHGEF19 (+49872); chr1:16533170-16533207/EPHA2 (−50607), ARHGEF19 (+5915); chr1:16553455-16553
469/ARHGEF19 (−14358), RSG1 (+10197); chr1:16553732-16553812/ARHGEF19 (−14668), RSG1 (+9887); chr
1:16554045-16554167/ARHGEF19 (−15002), RSG1 (+9553); chr1:16693976-16694028/RSG1 (−130343), SPAT
A21 (+69917); chr1:16767360-16767418/SPATA21 (−3470), NECAP2 (+222); chr1:16825858-16825876/NECAP
2 (+58700), NBPF1 (+114115); chr1:16970919-16970980/NBPF1 (−30968), MST1P9 (+120025); chr1:16974002-
16974084/NBPF1 (−34061), MST1P9 (+116932); chr1:16974638-16974676/NBPF1 (−34675), MST1P9 (+11631
8); chr1:16974784-16974806/NBPF1 (−34813), MST1P9 (+116180); chr1:17019473-17019717/NBPF1 (−79613),
MST1P9 (+71380); chr1:17033884-17033938/NBPF1 (−93929), MST1P9 (+57064); chr1:17066681-17066880/
NBPF1 (−126799), MST1P9 (+24194); chr1:17215440-17215476/MST1P9 (−124483), CROCC (−32987); chr1:17
446446-17446534/PADI2 (−542); chr1:17764306-17764355/RCC2 (+728); chr1:17764769-17764798/RCC2 (+2
75); chr1:18017856-18017907/ACTL8 (−63926), ARHGEF10L (+151552); chr1:18434311-18434366/IGSF21 (+9
9); chr1:18434539-18434705/IGSF21 (+382); chr1:18436999-18437043/KLHDC7A (−370403), IGSF21 (+2781);
chr1:18872535-18872691/PAX7 (−84887), KLHDC7A (+65189); chr1:18956611-18956738/PAX7 (−825); chr1:18
958229-18958358/PAX7 (+794); chr1:18959390-18959455/PAX7 (+1923), TAS1R2 (+226732); chr1:19029607-
19029659/PAX7 (+72133), TAS1R2 (+156522); chr1:19043067-19043114/PAX7 (+85591), TAS1R2 (+143064);
chr1:19043743-19043756/PAX7 (+86250), TAS1R2 (+142405); chr1:19110642-19110720/TAS1R2 (+75474), P
AX7 (+153181); chr1:19145857-19145925/TAS1R2 (+40264), PAX7 (+188391); chr1:19283333-19283381/ALD
H4A1 (−54064), UBR4 (+253389); chr1:19536544-19536560/UBR4 (+194); chr1:19600727-19600931/AKR7L
(−261); chr1:19615510-19615552/AKR7A3 (−251); chr1:19971439-19971472/HTR6 (−20324), NBL1 (+1733); chr1:
19992155-19992177/HTR6 (+386); chr1:19992313-19992417/HTR6 (+585); chr1:19992563-19992634/HTR6
(+819); chr1:20669848-20669896/VWA5B1 (+52460), CAMK2N1 (+142856); chr1:20692927-20692995/VWA5B1
(+75549), CAMK2N1 (+119767); chr1:20693106-20693136/VWA5B1 (+75709), CAMK2N1 (+119607); chr1:207
58203-20758297/CAMK2N1 (+54478), VWA5B1 (+140838); chr1:20810838-20810864/CAMK2N1 (+1877), VW
A5B1 (+193439); chr1:20811189-20811219/CAMK2N1 (+1524), VWA5B1 (+193792); chr1:20813197-20813270/
CAMK2N1 (−506); chr1:20879229-20879255/MUL1 (−44568), CDA (−36202); chr1:20879437-20879462/MUL1
(−44776), CDA (−35994); chr1:21619997-21620131/ECE1 (−3298); chr1:21671913-21671961/NBPF3 (−94694), E
CE1 (−55171); chr1:21754782-21754847/ECE1 (−138049), NBPF3 (−11816); chr1:21755148-21755187/ECE1
(−138402), NBPF3 (−11463); chr1:21763035-21763091/NBPF3 (−3568); chr1:21835733-21835765/ALPL (−109); c
hr1:21949101-21949127/RAP1GAP (+29234), ALPL (+113256); chr1:22109678-22109702/USP48 (−2); chr1:22
150158-22150183/LDLRAD2 (+11413), HSPG2 (+113579); chr1:22230557-22230636/HSPG2 (+33153), LDLR
AD2 (+91839); chr1:22379702-22379736/CDC42 (+599); chr1:22890180-22890189/EPHA8 (+181); chr1:23036
849-23036910/EPHB2 (−451); chr1:23037541-23037595/EPHB2 (+237); chr1:23280037-23280074/KDM1A (−65
885), EPHB2 (+242725); chr1:23280180-23280206/KDM1A (−65748), EPHB2 (+242862); chr1:23543794-23543
840/HTR1D (−22595), HNRNPR (+127036); chr1:23750467-23750535/TCEA3 (+760); chr1:23895042-23895062/
RPL11 (−123217), ID3 (−8767); chr1:24018253-24018289/RPL11 (+2); chr1:24127250-24127261/GALE (−1196);
chr1:24229435-24229535/FUCA1 (−34626), CNR2 (+10332); chr1:24286150-24286192/PNRC2 (−130); chr1:24
286404-24286446/PNRC2 (+124); chr1:24514552-24514568/IL28RA (−795); chr1:24882910-24882960/C1orf13
0 (+368); chr1:25071680-25071744/CLIC4 (−48); chr1:25258830-25258871/RUNX3 (−2081); chr1:25944438-259
44497/MAN1C1 (+509); chr1:25944639-25944655/MAN1C1 (+688); chr1:26097907-26098032/SEPN1 (−28697),
MAN1C1 (+154011); chr1:26201821-26201959/PAQR7 (−4146); chr1:26362261-26362312/SLC30A2 (+10317),
EXTL1 (+14016); chr1:26551625-26551728/CEP85 (−9016), CATSPER4 (+34558); chr1:26611720-26611776/
SH3BGRL3 (+5535), UBXN11 (+21447); chr1:26686401-26686447/AIM1L (−5803), ZNF683 (+12842); chr1:267
37883-26737907/LIN28A (+626); chr1:26827410-26827464/RPS6KA1 (−44906), HMGN2 (+28535); chr1:26948
206-26948270/ARID1A (−74284), RPS6KA1 (+75895); chr1:27160117-27160209/SFN (−29470), ZDHHC18 (+69
62); chr1:27286230-27286282/TRNP1 (−33939), NUDC (+38032); chr1:27328450-27328591/TRNP1 (+8326), S
LC9A1 (+152930); chr1:27481545-27481604/SLC9A1 (−124); chr1:27684181-27684240/MAP3K6 (+9126), SYT
L1 (+15728); chr1:27687148-27687522/MAP3K6 (+6002), SYTL1 (+18852); chr1:27693115-27693151/MAP3K6
(+204); chr1:27709609-27709757/CD164L2 (+122); chr1:27816367-27816423/WASF2 (+283); chr1:27816552-
27816581/WASF2 (+111); chr1:27895023-27895063/WASF2 (−78365), AHDC1 (+35100); chr1:27895384-2789
5491/WASF2 (−78760), AHDC1 (+34705); chr1:27961738-27961831/FGR (−58); chr1:28052394-28052491/IFI6
(−53719), STX12 (−47251); chr1:28157246-28157274/PPP1R8 (+8); chr1:28240713-28240793/RPA2 (+483); chr
1:28523842-28523916/PTAFR (−20424), DNAJC8 (+35663); chr1:28559145-28559213/ATPIF1 (−3423), DNAJC
8 (+363); chr1:28562180-28562219/DNAJC8 (−2658), ATPIF1 (−402); chr1:28562521-28562549/DNAJC8 (−2993),
ATPIF1 (−67); chr1:28586935-28586972/SESN2 (+991); chr1:28908150-28908234/RAB42 (−10815), TRNAU1
AP (+28663); chr1:29101930-29102028/OPRD1 (−36675), YTHDF2 (+38846); chr1:29213480-29213517/EPB41
(−104); chr1:29240814-29240835/EPB41 (+27222), TMEM200B (+209596); chr1:30815417-30815454/MATN1
(+380996); chr1:31538461-31538499/PUM1 (+84); chr1:31846725-31846761/FABP3 (−820); chr1:31867849-318
67886/FABP3 (−21945), SERINC2 (−18095); chr1:32180821-32180905/COL16A1 (−11095), BAI2 (+48785); chr1:
32226328-32226372/COL16A1 (−56582), BAI2 (+3298); chr1:32237546-32237638/BAI2 (−7944), SPOCD1 (+43
988); chr1:32479942-32479992/KHDRBS1 (+476); chr1:32671668-32671715/DCDC2B (−3003); chr1:32706690-
32706729/LCK (−33002), EIF3I (+18751); chr1:32716826-32716885/LCK (−22856), EIF3I (+28897); chr1:328272
92-32827307/TSSK3 (−562); chr1:33116692-33116715/RBBP4 (−45); chr1:33160877-33160910/SYNC (+7467),
RBBP4 (+44145); chr1:33220273-33220322/S100PBP (−62878), SYNC (−51937); chr1:33336598-33336635/YA
RS (−52984), FNDC5 (+1465); chr1:33352206-33352236/HPCA (+123); chr1:33438420-33438467/RNF19B (−81
58), AK2 (+64068); chr1:33772843-33772856/ZNF362 (+50676), PHC2 (+68344); chr1:33772957-33772986/ZN
F362 (+50798), PHC2 (+68222); chr1:33896824-33896927/PHC2 (−55682), ZSCAN20 (−41356); chr1:34631825-
34631871/CSMD2 (−973); chr1:34632501-34632613/CSMD2 (−1682); chr1:34642490-34642518/C1orf94 (−49);
chr1:34642668-34642712/C1orf94 (+137); chr1:35396049-35396107/DLGAP3 (−25094), LOC653160 (+48229);
chr1:36023133-36023186/NCDN (−233), KIAA0319L (−123); chr1:36023578-36023591/KIAA0319L (−548), NCD
N (+192); chr1:36028848-36029009/TFAP2E (−10042), NCDN (+5536); chr1:36184673-36184746/PSMB2 (−772
65), CLSPN (+50841); chr1:36235405-36235461/CLSPN (+118); chr1:36590505-36590532/COL8A2 (−24669),
TRAPPC3 (+24548); chr1:36615366-36615406/TRAPPC3 (−319); chr1:36626769-36626846/THRAP3 (−63209),
MAP7D1 (+5005); chr1:36787012-36787144/FAM176B (+2677), THRAP3 (+97061); chr1:36787260-36787358/
FAM176B (+2446), THRAP3 (+97292); chr1:36788105-36788308/FAM176B (+1548), THRAP3 (+98190); chr1:3
6788555-36788583/FAM176B (+1186), THRAP3 (+98552); chr1:36929716-36929748/MRPS15 (+308); chr1:37
498568-37498597/CSF3R (−549668), GRIK3 (+1261); chr1:37498845-37498888/GRIK3 (+977); chr1:37499781-
37499937/GRIK3 (−15); chr1:37500575-37500602/GRIK3 (−745); chr1:37980503-37980552/MEAF6 (−164); chr1:
38100493-38100533/RSPO1 (+82); chr1:38158286-38158335/CDCA8 (+152); chr1:38230243-38230282/EPHA
10 (+561); chr1:38260161-38260192/MANEAL (+403); chr1:38397446-38397478/MTF1 (−72170), INPP5B (+15
267); chr1:38510734-38510773/POU3F1 (+1696), UTP11L (+32370); chr1:38511800-38511821/POU3F1 (+639);
chr1:38512114-38512126/POU3F1 (+330); chr1:38601959-38601996/POU3F1 (−89528), RRAGC (+723362);
chr1:39044059-39044095/POU3F1 (−531627), RRAGC (+281263); chr1:39281997-39282073/POU3F1 (−76958
5), RRAGC (+43305); chr1:39283653-39283677/POU3F1 (−771215), RRAGC (+41675); chr1:39958053-399581
79/BMP8A (+798); chr1:39980365-39980407/BMP8A (+23068), PABPC4 (+62135); chr1:39980848-39980867/B
MP8A (+23540), PABPC4 (+61663); chr1:39981066-39981411/BMP8A (+23921), PABPC4 (+61282); chr1:4007
5758-40075770/PABPC4 (−33243), HEYL (+29584); chr1:40137607-40137646/NT5C1A (+83); chr1:40149745-4
0149784/NT5C1A (−12055), HPCAL4 (+7324); chr1:40235990-40236124/OXCT2 (+963); chr1:40236305-40236
450/OXCT2 (+642); chr1:40236601-40236683/OXCT2 (+378); chr1:40236988-40237013/OXCT2 (+19); chr1:40
253802-40253944/BMP8B (+660); chr1:40254231-40254279/BMP8B (+278); chr1:40254807-40254922/BMP8B
(−332); chr1:40255043-40255068/BMP8B (−523); chr1:40367594-40367609/MYCL1 (+85); chr1:40426168-4042
6241/CAP1 (−80050), MFSD2A (+5421); chr1:40505783-40505910/CAP1 (−408); chr1:41061665-41061734/ZNF
684 (+64467), RIMS3 (+69624); chr1:41131376-41131403/RIMS3 (−66); chr1:41349795-41350136/CTPS (−950
41), CITED4 (−21948); chr1:41445900-41445968/CTPS (+927); chr1:41823898-41823992/SCMH1 (−116130), E
DN2 (+126399); chr1:41847253-41847296/SCMH1 (−139460), EDN2 (+103069); chr1:41848889-41848942/SC
MH1 (−141101), EDN2 (+101428); chr1:41962195-41962280/EDN2 (−11894), HIVEP3 (+422258); chr1:4220469
7-42204851/EDN2 (−254430), HIVEP3 (+179722); chr1:42385900-42385942/HIVEP3 (−1425); chr1:42501064-4
2501120/GUCA2B (−118000), HIVEP3 (−116596); chr1:42506846-42506887/HIVEP3 (−122371), GUCA2B (−112
225); chr1:43232852-43232901/LEPRE1 (−122); chr1:43251031-43251075/ERMAP (−40196), LEPRE1 (−18298);
chr1:43251223-43251297/ERMAP (−39989), LEPRE1 (−18505); chr1:43533983-43534114/SLC2A1 (−109202),
EBNA1BP2 (+104192); chr1:43919528-43919541/HYI (+125); chr1:44031699-44031737/KDM4A (−84079), PTP
RF (+35171); chr1:44399150-44399194/ARTN (+180); chr1:44402394-44402415/IPO13 (−10073), ARTN (+3413);
chr1:44872850-44872917/RNF220 (+1924), TMEM53 (+267215); chr1:44873185-44873501/RNF220 (+2383),
TMEM53 (+266756); chr1:44874031-44874080/RNF220 (+3096), TMEM53 (+266043); chr1:44874523-448745
65/RNF220 (+3584), TMEM53 (+265555); chr1:44883246-44883751/RNF220 (+12539), TMEM53 (+256600); c
hr1:44884123-44884203/RNF220 (+13203), TMEM53 (+255936); chr1:45097941-45097970/TMEM53 (+42143),
RNF220 (+226996); chr1:45098149-45098213/TMEM53 (+41918), RNF220 (+227221); chr1:45249966-452500
08/BEST4 (+3439), RPS8 (+8741); chr1:45250162-45250192/BEST4 (+3249), RPS8 (+8931); chr1:45275838-4
5275969/PLK3 (+9868), PTCH2 (+32712); chr1:45792990-45793076/HPDL (+488); chr1:45805611-45805624/
MUTYH (+169), TOE1 (+276); chr1:46216320-46216357/IPP (+146); chr1:46632362-46632464/PIK3R3 (−34033),
TSPAN1 (−8336); chr1:46859790-46859827/FAAH (−130); chr1:46913877-46913886/DMBX1 (−58786), FAAH
(+53943); chr1:46914246-46914285/DMBX1 (−58402), FAAH (+54327); chr1:46951557-46951644/DMBX1 (−210
67), FAAH (+91662); chr1:46956616-46956684/DMBX1 (−16018), FAAH (+96711); chr1:46958796-46958809/D
MBX1 (−13865), FAAH (+98864); chr1:47009886-47009910/KNCN (+6989), DMBX1 (+37230); chr1:47010036-4
7010104/KNCN (+6817), DMBX1 (+37402); chr1:47082533-47082554/MOB3C (−1739); chr1:47134198-471342
50/ATPAF1-AS1 (−3273), ATPAF1 (−125); chr1:47694422-47694529/TAL1 (−4706), TAL1 (+967); chr1:47694928-
47694979/TAL1 (+489); chr1:47696521-47696620/TAL1 (−1128); chr1:47696965-47696986/TAL1 (−1533); chr1:
47697255-47697279/TAL1 (−1824); chr1:47697947-47698006/TAL1 (−2534); chr1:47882643-47882696/FOXE3
(+926); chr1:47899977-47900005/FOXD2 (−1698); chr1:47900136-47900264/FOXD2 (−1489); chr1:47904458-4
7904495/FOXD2 (+2788), LOC388630 (+558085); chr1:47904774-47905009/FOXD2 (+3203), LOC388630 (+55
7670); chr1:47905110-47905132/FOXD2 (+3432), LOC388630 (+557441); chr1:47910625-47910748/FOXD2
(+8998), LOC388630 (+551875); chr1:47911335-47911423/FOXD2 (+9690), LOC388630 (+551183); chr1:479118
31-47911977/FOXD2 (+10215), LOC388630 (+550658); chr1:48058793-48058946/FOXD2 (+157181), LOC388
630 (+403692); chr1:48175006-48175027/FOXD2 (+273328), LOC388630 (+287545); chr1:48463262-4846328
5/LOC388630 (−712); chr1:50680452-50680531/ELAVL4 (+105898), DMRTA2 (+208627); chr1:50799317-5079
9393/DMRTA2 (+89764), ELAVL4 (+224761); chr1:50880798-50880931/DMRTA2 (+8254), ELAVL4 (+306271);
chr1:50881317-50881426/DMRTA2 (+7747), ELAVL4 (+306778); chr1:50881957-50882143/DMRTA2 (+7069),
ELAVL4 (+307456); chr1:50883977-50884020/DMRTA2 (+5120), ELAVL4 (+309405); chr1:50884353-5088469
0/DMRTA2 (+4597), ELAVL4 (+309928); chr1:50884888-50885248/DMRTA2 (+4051), ELAVL4 (+310474); chr1:
50890649-50890795/DMRTA2 (−1603); chr1:50892284-50892336/DMRTA2 (−3191); chr1:50893423-50893518/
DMRTA2 (−4352); chr1:51810628-51810669/TTC39A (+136); chr1:52194878-52194940/OSBPL9 (+112363), N
RD1 (+149700); chr1:52195290-52195348/OSBPL9 (+112773), NRD1 (+149290); chr1:52834298-52834344/O
RC1 (+35822), ZFYVE9 (+226275); chr1:53308660-53308907/ZYG11A (+601); chr1:53528134-53528170/POD
N (+428); chr1:53528963-53528990/PODN (+1253), SLC1A7 (+79312); chr1:53686074-53686227/C1orf123 (+1
38); chr1:53703966-53704002/MAGOH (+298); chr1:54040869-54040940/DMRTB1 (+115833), GLIS1 (+15897
2); chr1:54203484-54203516/GLIS1 (−3623); chr1:54203673-54203727/GLIS1 (−3823); chr1:54411420-5441145
5/HSPB11 (−150); chr1:54518348-54518407/TCEANC2 (−896), TMEM59 (+733); chr1:54587512-54587534/CD
CP2 (+31920), TCEANC2 (+68249); chr1:54665620-54665659/MRPL37 (−200), CYB5RL (+106); chr1:5466603
0-54666097/CYB5RL (−318), MRPL37 (+224); chr1:54941131-54941169/ACOT11 (−72657), SSBP3 (−69058); c
hr1:57110663-57110714/PRKAA2 (−301); chr1:58715327-58715359/DAB1 (+868); chr1:58715855-58715978/D
AB1 (+294); chr1:59041846-59041919/OMA1 (−29437), TACSTD2 (+1283); chr1:59165706-59165743/MYSM1
(+22); chr1:60280341-60280394/HOOK1 (−165); chr1:60539349-60539399/C1orf87 (+68); chr1:61509226-61509
262/C1orf87 (−969802), NFIA (−38290); chr1:61519405-61519472/C1orf87 (−979997), NFIA (−28095); chr1:6220
8262-62208291/INADL (+128); chr1:62660769-62660785/USP1 (−241198), INADL (+452628); chr1:62661447-6
2661507/USP1 (−240498), INADL (+453328); chr1:63785309-63785375/FOXD3 (−3388); chr1:63785530-63785
592/FOXD3 (−3169); chr1:63785866-63785973/FOXD3 (−2810); chr1:63787249-63787282/FOXD3 (−1464); chr1:
63788827-63788860/FOXD3 (+114); chr1:63789792-63789849/ALG6 (−43440), FOXD3 (+1091); chr1:6378999
4-63790029/ALG6 (−43249), FOXD3 (+1282); chr1:63792179-63792231/ALG6 (−41056), FOXD3 (+3475); chr1:
63792649-63792797/ALG6 (−40538), FOXD3 (+3993); chr1:64240452-64240532/ROR1 (+802); chr1:65210646-
65210695/RAVER2 (−107); chr1:65210908-65210939/RAVER2 (+146); chr1:65533027-65533077/JAK1 (−10086
5), AK4 (−80180); chr1:65731599-65731658/LEPR (−154502), DNAJC6 (+1199); chr1:65991019-65991119/PDE
4B (−267787), LEPROT (+104938); chr1:65991561-65991605/PDE4B (−267273), LEPROT (+105452); chr1:659
91758-65991783/PDE4B (−267085), LEPROT (+105640); chr1:66257994-66258064/PDE4B (−827); chr1:66258
651-66258671/PDE4B (−195); chr1:67773981-67774115/IL12RB2 (+1001), SERBP1 (+122075); chr1:68298383-
68298403/GNG12 (+762); chr1:71172259-71172299/CTH (+295378), PTGER3 (+341212); chr1:72749700-727
49731/NEGR1 (−1311); chr1:75198767-75198840/TYW3 (−32), CRYZ (+288); chr1:75593969-75594000/LHX8
(−134); chr1:75595759-75595818/LHX8 (+1670), SLC44A5 (+481010); chr1:75595991-75596135/LHX8 (+1944),
SLC44A5 (+480736); chr1:75596859-75596929/LHX8 (+2775), SLC44A5 (+479905); chr1:75601119-75601187/
LHX8 (+7034), SLC44A5 (+475646); chr1:76190696-76190728/ACADM (+669); chr1:77333434-77333579/ST6
GALNAC5 (+321); chr1:77333992-77334168/ST6GALNAC5 (+894); chr1:77334386-77334408/ST6GALNAC5
(+1211), PIGK (+350735); chr1:77334757-77334795/ST6GALNAC5 (+1590), PIGK (+350356); chr1:78149314-7
8149403/ZZZ3 (−1016); chr1:78956792-78956874/PTGFR (+105); chr1:82266419-82266440/LPHN2 (+348); chr
1:82267678-82267739/LPHN2 (+1627); chr1:84326316-84326357/TTLL7 (+138496); chr1:85156177-85156237/
SSX2IP (+33); chr1:85358637-85358663/LPAR3 (+246); chr1:86042234-86042275/CYR61 (−4189); chr1:86043
397-86043466/CYR61 (−3012); chr1:86047024-86047064/CYR61 (+600); chr1:86081822-86081862/CYR61 (+3
5398), ZNHIT6 (+92274); chr1:86622685-86622770/COL24A1 (−574); chr1:86861605-86861650/ODF2L (+397);
chr1:87598103-87598318/LOC339524 (+531); chr1:87617313-87617385/LMO4 (−176802), LOC339524 (+1966
9); chr1:89990298-89990331/LRRC8B (−82); chr1:90228947-90228980/LRRC8D (−58516), LRRC8C (+130320);
chr1:91176597-91176691/BARHL2 (+6150), ZNF326 (+715966); chr1:91183611-91183775/BARHL2 (−899); ch
r1:91185309-91185347/BARHL2 (−2534); chr1:91190949-91190984/BARHL2 (−8173), ZNF644 (+296845); chr1:
91191235-91191289/BARHL2 (−8468), ZNF644 (+296550); chr1:91192577-91192681/BARHL2 (−9835), ZNF64
4 (+295183); chr1:91196195-91196225/BARHL2 (−13416), ZNF644 (+291602); chr1:91300907-91301161/BAR
HL2 (−118240), ZNF644 (+186778); chr1:91301739-91301814/BARHL2 (−118983), ZNF644 (+186035); chr1:91
306983-91307069/BARHL2 (−124232), ZNF644 (+180786); chr1:91966929-91966982/CDC7 (+552); chr1:92351
813-92351932/TGFBR3 (−37); chr1:94312394-94312456/BCAR3 (−165040), DNTTIP2 (+32337); chr1:94703078-
94703103/ARHGAP29 (+216); chr1:95392843-95392887/CNN3 (−130); chr1:98386567-98386618/DPYD (+22);
chr1:99469697-99469759/LPPR5 (+721); chr1:99470062-99470211/LPPR5 (+312); chr1:99470530-99470582/
LPPR5 (−107); chr1:99730323-99730351/LPPR4 (+489); chr1:100315730-100315854/AGL (+152); chr1:100715
383-100715426/DBT (+4); chr1:101004817-101004842/VCAM1 (−180366), GPR88 (+1102); chr1:106623467-10
6623545/PRMT6 (−975761); chr1:108023398-108023440/NTNG1 (+340790), VAV3 (+484126); chr1:108507077-
108507148/VAV3 (+432); chr1:108507376-108507494/VAV3 (+110); chr1:108507607-108507657/VAV3 (−87);
chr1:109506074-109506105/CLCC1 (+21); chr1:109584013-109584056/CLCC1 (−77924), TAF13 (+34589); chr
1:109633398-109633440/TMEM167B (+16); chr1:110026078-110026126/CYB561D1 (−10599), SYPL2 (+17002);
chr1:110527168-110527248/AHCYL1 (−179); chr1:110610328-110610423/ALX3 (+2946), FAM40A (+33135);
chr1:110610898-110611045/ALX3 (+2350), FAM40A (+33731); chr1:110611277-110611434/ALX3 (+1966), FA
M40A (+34115); chr1:110611966-110612129/ALX3 (+1274), FAM40A (+34807); chr1:110612692-110612963/A
LX3 (+494); chr1:110613273-110613301/ALX3 (+35); chr1:110626528-110626677/UBL4B (−28459), ALX3 (−132
81); chr1:110693497-110693736/SLC6A17 (+485); chr1:110753220-110753325/KCNC4 (−63); chr1:110753894-
110753957/KCNC4 (+590); chr1:110754357-110754632/RBM15 (−127450), KCNC4 (+1159); chr1:110754736-1
10754761/RBM15 (−127196), KCNC4 (+1413); chr1:111216979-111217139/KCNA3 (+596); chr1:111217892-11
1217923/KCNA3 (−253); chr1:111813739-111813750/CHIA (−19739), CHI3L2 (+43464); chr1:111991844-11199
1885/WDR77 (−35), ATP5F1 (+122); chr1:112058194-112058222/RAP1A (−104197), ADORA3 (−11465); chr1:11
2058347-112058396/RAP1A (−104033), ADORA3 (−11629); chr1:112162239-112162273/RAP1A (−149); chr1:11
2281121-112281180/DDX20 (−17039), RAP1A (+118746); chr1:112532077-112532114/KCND3 (−319); chr1:113
052166-113052213/WNT2B (+820); chr1:113286689-113286742/FAM19A3 (+23527), SLC16A1 (+212259); chr
1:113286892-113287134/FAM19A3 (+23824), SLC16A1 (+211962); chr1:113933279-113933305/MAGI3 (−183);
chr1:114448128-114448169/AP4B1 (−624), DCLRE1B (+234); chr1:114695737-114695799/SYT6 (+704); chr1:
114696183-114696349/SYT6 (+206); chr1:114696464-114696540/SYT6 (−30); chr1:115259504-115259566/NR
AS (−20); chr1:115300208-115300249/CSDE1 (+442); chr1:115300536-115300575/CSDE1 (+115); chr1:115632
640-115632692/TSPAN2 (−545); chr1:115880795-115880872/NGF (+23); chr1:115881043-115881248/NGF (−2
89); chr1:117602388-117602424/TTF2 (−543); chr1:117602625-117602704/TTF2 (−284); chr1:117664354-1176
64407/TRIM45 (+30); chr1:118471804-118471866/WDR3 (−537), GDAP2 (+467); chr1:119521927-119521978/
SPAG17 (−794105), TBX15 (+10226); chr1:119522435-119522565/SPAG17 (−794652), TBX15 (+9679); chr1:11
9522854-119522925/SPAG17 (−795042), TBX15 (+9289); chr1:119527064-119527236/SPAG17 (−799302), TB
X15 (+5029); chr1:119527788-119527820/SPAG17 (−799956), TBX15 (+4375); chr1:119529337-119529378/SP
AG17 (−801510), TBX15 (+2821); chr1:119529912-119530005/SPAG17 (−802111), TBX15 (+2220); chr1:11953
0149-119530201/SPAG17 (−802327), TBX15 (+2004); chr1:119530508-119530553/SPAG17 (−802683), TBX15
(+1648); chr1:119532043-119532317/TBX15 (−1); chr1:119532772-119532924/TBX15 (−669); chr1:119535721-
119535907/TBX15 (−3635); chr1:119542200-119542216/TBX15 (−10029), WARS2 (+141087); chr1:119542994-
119543069/TBX15 (−10853), WARS2 (+140263); chr1:119543215-119543273/TBX15 (−11065), WARS2 (+1400
51); chr1:119549735-119549914/TBX15 (−17646), WARS2 (+133470); chr1:119550721-119551013/TBX15 (−18
688), WARS2 (+132428); chr1:120190450-120190519/ZNF697 (−95); chr1:120906200-120906245/NOTCH2 (−2
93906), FCGR1B (+29721); chr1:121137945-121137965/FCGR1B (−202011); chr1:121138946-121139009/FCG
R1B (−203034); chr1:121261239-121261403/FCGR1B (−325377); chr1:143912982-143913019/PPIAL4G (−1451
20), PPIAL4A (+451245); chr1:144594151-144594207/PPIAL4A (−229933), PDE4DIP (+400854); chr1:1450754
83-145075577/PDE4DIP (−80497), SEC22B (−20877); chr1:145075788-145075897/PDE4DIP (−80810), SEC22B
(−20564); chr1:145562808-145562833/PIAS3 (−13167), ITGA10 (+37831); chr1:145575168-145575231/PIAS3
(−788); chr1:146544203-146544272/PRKAB2 (+99891), KIAA1245 (+210048); chr1:146552529-146552566/PRK
AB2 (+91581), KIAA1245 (+218358); chr1:147142740-147142781/ACP6 (−127); chr1:147532653-147532666/N
BPF11 (+91809), GPR89B (+132154); chr1:147752953-147753060/NBPF11 (−128538), PPIAL4A (+202412); ch
r1:147775413-147775495/NBPF11 (−150985), PPIAL4A (+179965); chr1:147775887-147776019/NBPF11 (−151
484), PPIAL4A (+179466); chr1:147806865-147806906/NBPF11 (−182417), PPIAL4A (+148533); chr1:1478071
41-147807168/NBPF11 (−182686), PPIAL4A (+148264); chr1:148241136-148241157/NBPF15 (−319700), PPIA
L4F (−38611); chr1:148247950-148248139/NBPF15 (−312802), PPIAL4F (−45509); chr1:148901829-148901905/
PPIAL4B (−651136), PPIAL4F (+95852); chr1:149146950-149147117/PPIAL4B (−405969), PPIAL4F (+341019);
chr1:149147470-149147509/PPIAL4B (−405513), PPIAL4F (+341475); chr1:149156198-149156460/PPIAL4B
(−396674), PPIAL4F (+350314); chr1:149194465-149194595/PPIAL4B (−358473), PPIAL4F (+388515); chr1:1496
08326-149608355/FCGR1A (−145909), PPIAL4B (+55338); chr1:150039729-150039791/VPS45 (+418); chr1:15
0121581-150121636/PLEKHO1 (−561); chr1:150487531-150487603/ADAMTSL4 (−34331), ECM1 (+7080); chr1:
150951761-150951855/CERS2 (−4329), ANXA9 (−2691); chr1:150980816-150980853/PRUNE (−138), FAM63A
(+19); chr1:151103905-151104036/SEMA6C (+15169), GABPB2 (+60891); chr1:151118185-151118236/SEMA6
C (+929); chr1:151300049-151300116/PI4KB (+108); chr1:151319341-151319406/RFX5 (+395); chr1:1518104
38-151810653/RORC (−6198), THEM5 (+15627); chr1:151810815-151810937/RORC (−6528), THEM5 (+15297);
chr1:151811160-151811259/RORC (−6862), THEM5 (+14963); chr1:151811939-151811996/RORC (−7620), T
HEM5 (+14205); chr1:152080035-152080074/TCHHL1 (−18515), TCHH (+6532); chr1:152080962-152081017/T
CHHL1 (−19450), TCHH (+5597); chr1:152082054-152082178/TCHHL1 (−20576), TCHH (+4471); chr1:1521615
07-152161520/RPTN (−29810), HRNR (+35155); chr1:152487957-152487999/LCE5A (+4658), LCE3E (+51270);
chr1:153233586-153233649/LOR (+1439), PGLYRP3 (+49576); chr1:153233862-153233925/LOR (+1715), P
GLYRP3 (+49300); chr1:153234048-153234099/LOR (+1895), PGLYRP3 (+49120); chr1:153643197-15364323
4/ILF2 (+263); chr1:153650810-153650908/NPR1 (−305); chr1:153700216-153700255/INTS3 (−331); chr1:1537
00398-153700438/INTS3 (−149); chr1:153963252-153963300/RAB13 (−4470), RPS27 (+37); chr1:154297990-1
54298038/ATP8B2 (−2262); chr1:154298153-154298253/ATP8B2 (−2073); chr1:154474778-154474987/TDRD1
0 (+188); chr1:154475201-154475370/TDRD10 (+591); chr1:154540419-154540455/CHRNB2 (+180); chr1:154
971620-154971706/ZBTB7B (−3539); chr1:155022988-155023026/ADAM15 (−755); chr1:155023698-155023736/
ADAM15 (−45); chr1:155054922-155054944/EFNA1 (−45416), EFNA3 (+3585); chr1:155139428-155139547/DP
M3 (−26605), KRTCAP2 (+6316); chr1:155145342-155145416/TRIM46 (−981), KRTCAP2 (+425); chr1:1551458
95-155145936/TRIM46 (−444), KRTCAP2 (−112); chr1:155147262-155147320/KRTCAP2 (−1487), TRIM46 (+93
1); chr1:155163239-155163286/MUC1 (−557); chr1:155232041-155232078/SCAMP3 (+116); chr1:155242901-1
55242948/HCN3 (−4449), CLK2 (+356); chr1:155265440-155265457/PKLR (+5776), HCN3 (+18075); chr1:1552
90878-155290940/RUSC1 (+269); chr1:156046658-156046681/MEX3A (+5119), RAB25 (+15704); chr1:156051
245-156051284/MEX3A (+524); chr1:156131108-156131150/SLC25A44 (−32601), SEMA4A (+7807); chr1:1562
61339-156261354/C1orf85 (+4102), TMEM79 (+7277); chr1:156391216-156391242/C1orf61 (+8111), RHBG
(+52226); chr1:156405415-156405634/C1orf61 (−6185), MEF2D (+65004); chr1:156406071-156406104/C1orf61
(−6748), MEF2D (+64441); chr1:156475098-156475176/MEF2D (−4608); chr1:156595053-156595080/BCAN (−16
673), HAPLN2 (+5981); chr1:156595184-156595221/BCAN (−16537), HAPLN2 (+6117); chr1:156611573-15661
1643/BCAN (−132); chr1:156611944-156611993/BCAN (+229); chr1:156612417-156612468/BCAN (+703); chr1:
156627019-156627083/BCAN (+15311), NES (+20138); chr1:156646597-156646634/NES (+573); chr1:156698
409-156698430/ISG20L2 (−715), RRNAD1 (+157); chr1:156698692-156698747/ISG20L2 (−1015), RRNAD1 (+4
57); chr1:156814953-156815030/SH2D2A (−28352), INSRR (+13720); chr1:156815240-156815273/SH2D2A (−2
8617), INSRR (+13455); chr1:156830192-156830265/INSRR (−1517), NTRK1 (−442); chr1:156862981-1568630
61/PEAR1 (−502); chr1:156863486-156863640/PEAR1 (+40); chr1:156893765-156893791/PEAR1 (+30255), A
RHGEF11 (+121384); chr1:156893979-156894012/PEAR1 (+30473), ARHGEF11 (+121166); chr1:156897155-
156897211/PEAR1 (+33660), ARHGEF11 (+117979); chr1:157015354-157015395/ARHGEF11 (−213); chr1:157
108362-157108382/ETV3 (+11); chr1:157164713-157164942/ETV3 (−56445), FCRL5 (+357482); chr1:1579634
26-157963443/KIRREL (+372); chr1:159141156-159141216/CADM3 (−191); chr1:159141400-159141432/CADM
3 (+39); chr1:159141544-159141599/CADM3 (+195); chr1:159158369-159158471/DARC (−16629), CADM3 (+1
7043); chr1:159750646-159750677/DUSP23 (−97); chr1:159824301-159824348/VSIG8 (+8122), SLAMF8 (+278
46); chr1:160053688-160053727/KCNJ9 (+2348), IGSF8 (+14910); chr1:160313036-160313056/NCSTN (−17),
COPA (+308); chr1:161008341-161008733/F11R (−17404), USF1 (+7220); chr1:161039305-161039377/ARHG
AP30 (+419); chr1:161102320-161102383/DEDD (−96); chr1:161136510-161136567/PPOX (+358); chr1:16114
7104-161147155/B4GALT3 (+628); chr1:161275579-161275639/NR1I3 (−67609), MPZ (+4153); chr1:16142709
8-161427179/FCGR2A (−48066), SDHC (+142973); chr1:161494028-161494160/HSPA6 (+58); chr1:162760320-
162760355/HSD17B7 (−158); chr1:164290672-164290723/PBX1 (−237899), NUF2 (+998975); chr1:165204697-
165204732/LMX1A (+120763), PBX1 (+676118); chr1:165321787-165321942/LMX1A (+3613), PBX1 (+793268);
chr1:165322397-165322515/LMX1A (+3022), PBX1 (+793859); chr1:165323519-165323697/LMX1A (+1870),
PBX1 (+795011); chr1:165325357-165325394/LMX1A (+102); chr1:165326205-165326296/LMX1A (−773); chr1:
165600060-165600140/MGST3 (−10); chr1:165796721-165796731/UCK2 (−6); chr1:166134891-166134933/PO
GK (−673812), UCK2 (+338180); chr1:166845626-166845653/TADA1 (+14); chr1:166890502-166890534/TADA
1 (−44864), ILDR2 (+54043); chr1:166916921-166916936/TADA1 (−71275), ILDR2 (+27632); chr1:166917085-1
66917117/TADA1 (−71447), ILDR2 (+27460); chr1:167408552-167408657/CD247 (+79242), POU2F1 (+218539);
chr1:167683520-167683573/CREG1 (−160491), MPZL1 (−7640); chr1:167789443-167789489/ADCY10 (+939
87), MPZL1 (+98279); chr1:167906795-167906830/BRP44 (−506); chr1:169396689-169396730/SLC19A2 (+584
98), BLZF1 (+59516); chr1:169429656-169429697/SLC19A2 (+25531), BLZF1 (+92483); chr1:170500941-1705
00993/GORAB (−296); chr1:170630047-170630085/PRRX1 (−3247); chr1:170636193-170636326/C1orf129 (−26
8352), PRRX1 (+2947); chr1:170640625-170640664/C1orf129 (−263967), PRRX1 (+7332); chr1:171811287-17
1811320/DNM3 (+683); chr1:172413276-172413317/PIGC (−67); chr1:172502037-172502085/C1orf9 (−199); ch
r1:173446541-173446599/PRDX6 (+84); chr1:173793676-173793729/CENPL (−433), DARS2 (−94); chr1:17383
6894-173836941/ZBTB37 (−575); chr1:173991522-173991550/RABGAP1L (−137016), RC3H1 (−29326); chr1:17
3991670-173991713/RABGAP1L (−136860), RC3H1 (−29482); chr1:174129036-174129082/RABGAP1L (+507);
chr1:174969455-174969482/CACYBP (+577); chr1:175568405-175568494/TNR (+144302), TNN (+531456); ch
r1:176867049-176867110/ASTN1 (+266944), PAPPA2 (+434773); chr1:177133587-177133689/ASTN1 (+386);
chr1:177140174-177140304/FAM5B (−394); chr1:178456021-178456063/C1orf49 (−26170), RASAL2 (+393178);
chr1:178456229-178456271/C1orf49 (−25962), RASAL2 (+393386); chr1:179712339-179712410/FAM163A (+7
7); chr1:179712554-179712567/FAM163A (+263); chr1:179712734-179712830/FAM163A (+484); chr1:1797131
75-179713518/FAM163A (+1049), TOR1AIP2 (+133594); chr1:179796745-179796820/TOR1AIP2 (+50158), FA
M163A (+84485); chr1:179851482-179851494/TOR1AIP2 (−4547), TOR1AIP2 (−4547), TOR1AIP1 (+68); chr1:1
80123687-180123700/QSOX1 (−274); chr1:180202486-180202648/LHX4 (+3134), ACBD6 (+269455); chr1:180
203608-180203633/LHX4 (+4188), ACBD6 (+268401); chr1:180204620-180204897/LHX4 (+5326), ACBD6 (+2
67263); chr1:180601741-180601806/XPR1 (+628); chr1:180881835-180881864/STX6 (+110196), XPR1 (+2807
04); chr1:180991756-180991822/STX6 (+257); chr1:181074295-181074333/CACNA1E (−378372), MR1 (+7175
3); chr1:181287200-181287267/CACNA1E (−165452), MR1 (+284673); chr1:181452986-181453050/CACNA1E
(+332); chr1:182025892-182025928/ZNF648 (+4937), CACNA1E (+573224); chr1:182026429-182026506/ZNF6
48 (+4379), CACNA1E (+573782); chr1:182359697-182359742/GLUL (+819); chr1:182360203-182360247/GLU
L (+314); chr1:182584340-182584403/RGS16 (−10824), RGS8 (+57695); chr1:182921896-182921930/LAMC1
(−70682), DHX9 (+113474); chr1:182922125-182922163/LAMC1 (−70451), DHX9 (+113705); chr1:183155477-18
3155524/LAMC2 (+327); chr1:183386319-183386598/NMNAT2 (+1175), LAMC2 (+231285); chr1:183386827-1
83386946/NMNAT2 (+747); chr1:183387623-183387662/NMNAT2 (−9); chr1:183440578-183440605/SMG7 (−91
4); chr1:184122416-184122513/TSEN15 (+101654), EDEM3 (+601576); chr1:184942823-184942877/FAM129A
(+832); chr1:186344196-186344241/C1orf27 (−4680), TPR (+238); chr1:190447300-190447388/FAM5C (−585);
chr1:196847239-196847297/CFHR2 (−65666), CFHR1 (+58407); chr1:197115787-197115840/ASPM (+10); chr
1:197170352-197170406/ZBTB41 (−707); chr1:197744530-197744581/DENND1B (+67); chr1:197879661-1978
79716/DENND1B (−135066), LHX9 (−6828); chr1:197883137-197883189/LHX9 (−3354); chr1:197887067-19788
7146/LHX9 (+590); chr1:197887457-197887706/NEK7 (−238526), LHX9 (+1065); chr1:197888122-197888180/
NEK7 (−237957), LHX9 (+1634); chr1:197898262-197898343/NEK7 (−227805), LHX9 (+11786); chr1:20000490
8-200005016/FAM58BP (−177694), NR5A2 (+8192); chr1:200005189-200005253/FAM58BP (−177435), NR5A2
(+8451); chr1:200005355-200005431/FAM58BP (−177263), NR5A2 (+8623); chr1:200010525-200010649/FAM5
8BP (−172069), NR5A2 (+13817); chr1:200211892-200211942/FAM58BP (+29261), ZNF281 (+167249); chr1:2
00247655-200247745/FAM58BP (+65044), ZNF281 (+131466); chr1:200271341-200271373/FAM58BP (+8870
1), ZNF281 (+107809); chr1:200589714-200589747/KIF14 (+131); chr1:200638929-200638975/DDX59 (+174);
chr1:200842755-200842866/GPR25 (+645); chr1:200992738-200992796/KIF21B (+61); chr1:201084190-20108
4226/CACNA1S (−2514); chr1:201437664-201437682/PHLDA3 (+626); chr1:201450223-201450300/PHLDA3
(−11963), CSRP1 (+25705); chr1:201708835-201708891/IPO9 (−89425), NAV1 (+91413); chr1:201924492-20192
4571/TIMM17A (−87); chr1:201951865-201951934/RNPEP (+134); chr1:202130680-202130727/PTPN7 (+12); c
hr1:202168601-202168629/LGR6 (+5497), UBE2T (+142479); chr1:202678881-202678923/SYT2 (+649); chr1:
202780427-202780478/KDM5B (−2904); chr1:202780592-202780634/KDM5B (−3064); chr1:202968746-202968
818/CYB5R1 (−32378), TMEM183B (−7752); chr1:202996231-202996304/PPFIA4 (−7344), TMEM183B (+19734);
chr1:203044861-203045073/MYOG (+10410), PPFIA4 (+41355); chr1:203045220-203045250/MYOG (+10142),
PPFIA4 (+41623); chr1:203045509-203045696/MYOG (+9774), PPFIA4 (+41991); chr1:203296839-2032968
71/BTG2 (+22191), FMOD (+23434); chr1:203764634-203764721/ZBED6 (−1973), ZC3H11A (−73); chr1:203870
670-203870687/SOX13 (−171567), SNRPE (+39939); chr1:204653596-204653792/LRRN2 (+903); chr1:205180
345-205180410/DSTYK (+349); chr1:205290252-205290331/NUAK2 (+591); chr1:205312912-205312961/NUA
K2 (−22054), LEMD1 (+78277); chr1:205600493-205600518/SLC45A3 (+49124), MFSD4 (+62394); chr1:20668
0210-206680247/RASSF5 (−650); chr1:206837298-206837397/MAPKAPK2 (−21017), DYRK3 (+28467); chr1:20
7669273-207669321/CR1 (−176); chr1:207669788-207669828/CR1 (+335); chr1:207818397-207818433/CR1L
(−43); chr1:207818547-207818598/CR1L (+115); chr1:207818703-207818745/CR1L (+266); chr1:207842948-20
7843005/CD46 (−82406), CR1L (+24519); chr1:208042154-208042180/CD34 (+42516), CD46 (+116784); chr1:
208084004-208084031/CD34 (+665); chr1:208132523-208132675/CD34 (−47916), PLXNA2 (+285066); chr1:20
8132800-208132879/CD34 (−48157), PLXNA2 (+284825); chr1:208133086-208133143/CD34 (−48432), PLXNA
2 (+284550); chr1:208338101-208338196/CD34 (−253466), PLXNA2 (+79516); chr1:208417859-208417907/PL
XNA2 (−218); chr1:209405049-209405087/PLXNA2 (−987403), CAMK1G (−351977); chr1:209405301-20940531
4/PLXNA2 (−987643), CAMK1G (−351737); chr1:209848576-209848614/G0S2 (−75); chr1:209920995-20992104
6/TRAF3IP3 (−8373), HSD11B1 (+42885); chr1:209943069-209943168/TRAF3IP3 (+13725), IRF6 (+36401); chr
1:209979612-209979634/IRF6 (−103); chr1:210111211-210111283/SYT14 (−291); chr1:210111422-210111796/
SYT14 (+71); chr1:210111923-210112036/SYT14 (+442); chr1:211432060-211432123/RCOR3 (−616); chr1:211
848603-211848657/NEK2 (+342); chr1:212688095-212688359/ATF3 (−93743), NENF (+81998); chr1:21287372
4-212873750/BATF3 (−410); chr1:213124623-213124668/VASH2 (+759); chr1:214153408-214153558/PROX1
(−8377), RPS6KC1 (+928895); chr1:214156083-214156134/PROX1 (−5751), RPS6KC1 (+931521); chr1:214156
279-214156394/PROX1 (−5523), RPS6KC1 (+931749); chr1:214158911-214159036/PROX1 (−2886); chr1:2143
60859-214360926/SMYD2 (−93672), PROX1 (+199033); chr1:215256873-215256953/KCNK2 (+353); chr1:2169
77604-216977639/ESRRG (−80808), GPATCH2 (+826787); chr1:217307266-217307384/ESRRG (−410511), GP
ATCH2 (+497084); chr1:217310297-217310395/ESRRG (−413532), GPATCH2 (+494063); chr1:218520063-218
520095/LYPLAL1 (−827113), TGFB2 (+1403); chr1:218520292-218520309/LYPLAL1 (−826891), TGFB2 (+1625);
chr1:220101211-220101370/SLC30A10 (+702); chr1:220101613-220101674/SLC30A10 (+349); chr1:220101
804-220101864/SLC30A10 (+159); chr1:221049979-221050667/HLX (−2420); chr1:221053210-221053255/HLX
(+490); chr1:221914974-221915018/DUSP10 (+520); chr1:223190806-223190867/TLR5 (+125787), DISP1 (+2
02406); chr1:223316608-223316633/TLR5 (+3); chr1:223936753-223936995/CAPN2 (+36755), TP53BP2 (+96
800); chr1:224180453-224180478/DEGS1 (−190444), TP53BP2 (−146792); chr1:224804296-224804395/CNIH3
(+167); chr1:224804687-224804846/CNIH3 (+588); chr1:224805116-224805197/CNIH3 (+978); chr1:22480575
2-224805848/DNAH14 (−311556), CNIH3 (+1621); chr1:225655018-225655149/LBR (−39269), ENAH (+185761);
chr1:225655506-225655609/LBR (−39743), ENAH (+185287); chr1:225665781-225665834/LBR (−49993), ENA
H (+175037); chr1:225840035-225840077/ENAH (+789); chr1:225997626-225997661/EPHX1 (−153); chr1:2259
98612-225998735/EPHX1 (+877); chr1:226075435-226075467/TMEM63A (−5031), LEFTY1 (+1395); chr1:2260
99205-226099220/LEFTY1 (−22367), PYCR2 (+12752); chr1:226111592-226111646/PYCR2 (+346); chr1:2261
87085-226187121/H3F3B (−63305), LEFTY2 (−58020); chr1:226271413-226271508/H3F3B (+21053), ACBD3
(+102962); chr1:226298171-226298214/H3F3B (+47785), ACBD3 (+76230); chr1:226374647-226374683/ACBD
3 (−242); chr1:226411224-226411246/MIXL1 (−148); chr1:226595775-226595812/PARP1 (+7); chr1:226613283-
226613329/C1orf95 (−123195), PARP1 (−17505); chr1:226737755-226737804/C1orf95 (+1279), ITPKB (+18909
6); chr1:226924830-226924900/ITPKB (+2011), C1orf95 (+188364); chr1:227506012-227506042/CDC42BPA
(−201); chr1:227729683-227729829/CDC42BPA (−223930), ZNF678 (−21464); chr1:227922601-227922654/SNAP
47 (−69); chr1:228135304-228135362/WNT9A (+343); chr1:228194411-228194570/WNT3A (−232); chr1:228247
134-228247144/ARF1 (−23222), WNT3A (+52416); chr1:228270821-228270849/ARF1 (+474); chr1:228345277-
228345323/IBA57 (−8129), GJC2 (+7885); chr1:228353402-228353495/IBA57 (+20); chr1:228401735-2284017
59/OBSCN (+5886), TRIM11 (+192770); chr1:228401885-228401938/OBSCN (+6051), TRIM11 (+192605); chr
1:228566079-228566109/TRIM11 (+28423), OBSCN (+170233); chr1:228566526-228566547/TRIM11 (+27980),
OBSCN (+170676); chr1:228633950-228633983/HIST3H3 (−20941), HIST3H2A (+11593); chr1:228645244-22
8645305/HIST3H2BB (−533), HIST3H2A (+285); chr1:228651902-228652206/RNF187 (−23014), HIST3H2BB
(+6246); chr1:228652453-228652508/RNF187 (−22587), HIST3H2BB (+6673); chr1:228784040-228784106/RHO
U (−86751), RNF187 (+109005); chr1:228786156-228786236/RHOU (−84628), RNF187 (+111128); chr1:229126
191-229126268/RAB4A (−280649), RHOU (+255406); chr1:229542953-229543220/C1orf96 (−64399), ACTA1
(+26756); chr1:229567277-229567369/C1orf96 (−88635), ACTA1 (+2520); chr1:229567993-229568157/C1orf96
(−89387), ACTA1 (+1768); chr1:229568704-229568780/C1orf96 (−90054), ACTA1 (+1101); chr1:230193557-2301
93667/GALNT2 (−9344), URB2 (+431631); chr1:230202562-230202575/GALNT2 (−387); chr1:230202913-23020
2921/GALNT2 (−39); chr1:230410149-230410215/PGBD5 (+103209), GALNT2 (+207226); chr1:231113961-231
114005/ARV1 (−840), TTC13 (+635); chr1:231114594-231114640/ARV1 (−206), TTC13 (+1); chr1:231175649-2
31175692/TRIM67 (−123003), ARV1 (+60848); chr1:231176765-231176816/TRIM67 (−121883), ARV1 (+61968);
chr1:231296606-231296658/TRIM67 (−2042); chr1:231296784-231296817/TRIM67 (−1873); chr1:231298708-2
31298924/TRIM67 (+142); chr1:231299252-231299325/TRIM67 (+615); chr1:231299459-231299478/TRIM67
(+795); chr1:232765537-232765595/NTPCR (−320804), SIPA1L2 (−114323); chr1:232941599-232941622/SIPA1
L2 (−290368), NTPCR (−144759); chr1:233749731-233749768/KCNK1 (0); chr1:234040614-234040673/SLC35F
3 (−35); chr1:234040974-234041148/SLC35F3 (+382); chr1:234041361-234041415/SLC35F3 (+709); chr1:2340
41659-234041794/C1orf31 (−467702), SLC35F3 (+1048); chr1:234614344-234614388/TARBP1 (+483); chr1:23
5039934-235040018/IRF2BP2 (−294705), TOMM20 (+252280); chr1:235105805-235105855/IRF2BP2 (−360559),
TOMM20 (+186426); chr1:235586844-235586983/TBCE (+56186), B3GALNT2 (+80867); chr1:236227744-23
6227769/NID1 (+724); chr1:236227921-236228021/NID1 (+510); chr1:236228624-236228705/NID1 (−184); chr1:
236558517-236558547/EDARADD (+852); chr1:236559207-236559256/LGALS8 (−127507), EDARADD (+1552);
chr1:236849506-236850197/ACTN2 (+82); chr1:236959272-236959305/MTR (+708); chr1:237205224-23720
5354/RYR2 (−413); chr1:237205634-237205671/RYR2 (−49); chr1:237205804-237206101/RYR2 (+251); chr1:23
7206340-237206508/RYR2 (+722); chr1:240160858-240160916/FMN2 (−94298), CHRM3 (+368514); chr1:2401
61086-240161122/FMN2 (−94081), CHRM3 (+368731); chr1:240256602-240256624/FMN2 (+1428), GREM2
(+518849); chr1:240256905-240257029/FMN2 (+1782), GREM2 (+518495); chr1:240775313-240775378/GREM2
(+116); chr1:242011232-242011273/EXO1 (−240); chr1:242688034-242688080/PLD5 (−59); chr1:242688244-2
42688277/PLD5 (−263); chr1:243053672-243053716/PLD5 (−365696), CEP170 (+365014); chr1:243417911-243
417953/SDCCAG8 (−1375), CEP170 (+776); chr1:243601336-243601428/SDCCAG8 (+182075), AKT3 (+40520
2); chr1:243646764-243646877/SDCCAG8 (+227514), AKT3 (+359763); chr1:244013192-244013265/ZNF238
(−201332), AKT3 (−6645); chr1:244081062-244081077/ZNF238 (−133491), AKT3 (−74486); chr1:244817035-2448
17069/FAM36A (−181587), C1orf101 (+192379); chr1:245850554-245850639/KIF26B (+532310), SMYD3 (+820
047); chr1:246670510-246670544/SMYD3 (+117); chr1:246952346-246952382/SCCPDH (+64986), AHCTF1
(+142362); chr1:247171181-247171251/ZNF695 (+179); chr1:247275683-247275715/ZNF669 (−8025), ZNF124
(+59619); chr1:247291034-247291106/ZNF669 (−23396), ZNF124 (+44248); chr1:247373691-247373744/VN1R
5 (−45656), ZNF124 (−38400); chr1:247496058-247496128/ZNF496 (−1048); chr1:247511391-247511461/NLRP
3 (−68032), ZNF496 (−16381); chr1:247802702-247802810/OR13G1 (+33587), OR2G3 (+33868); chr1:2480204
49-248020515/TRIM58 (−19); chr1:248020631-248020720/TRIM58 (+175); chr1:249120192-249120240/LOC64
6627 (−217065), ZNF672 (−12314); chr10:681263-681420/DIP2C (+54266), ZMYND11 (+455408); chr10:681634-
681694/DIP2C (+53944), ZMYND11 (+455730); chr10:735224-735245/DIP2C (+373); chr10:830780-830815/DI
P2C (−95190), LARP4B (+100904); chr10:836303-836405/DIP2C (−100746), LARP4B (+95348); chr10:850908-8
51006/DIP2C (−115349), LARP4B (+80745); chr10:1094975-1095030/IDI1 (+58); chr10:1454224-1454400/IDI1
(−359251), ADARB2 (+325358); chr10:1651298-1651359/IDI1 (−556268), ADARB2 (+128341); chr10:1774913-17
74954/IDI1 (−679873), ADARB2 (+4736); chr10:1779083-1779111/ADARB2 (+573); chr10:3138409-3138450/P
FKP (+28718), PITRM1 (+76603); chr10:3155277-3155318/PFKP (+45586), PITRM1 (+59735); chr10:3214909-
3214966/PITRM1 (+95); chr10:5648580-5648693/ASB13 (+59921), CALML3 (+81713); chr10:5726789-572681
8/ASB13 (−18246), GDI2 (+128708); chr10:6245042-6245101/PFKFB3 (+232); chr10:7449817-7449839/PRKC
Q (−827574), SFMBT2 (+3620); chr10:7450244-7450490/PRKCQ (−828113), SFMBT2 (+3081); chr10:7453657-
7453902/SFMBT2 (−332); chr10:7454613-7454628/SFMBT2 (−1173); chr10:7454813-7454891/SFMBT2 (−1404);
chr10:7645840-7645935/SFMBT2 (−192440), ITIH5 (+63073); chr10:7708857-7708954/ITIH5 (+55); chr10:7709
941-7710011/ITIH5 (−1015); chr10:8075945-8076053/GATA3 (−20668), TAF3 (+215326); chr10:8076177-80763
00/GATA3 (−20428), TAF3 (+215566); chr10:8079390-8079432/GATA3 (−17256), TAF3 (+218738); chr10:80903
78-8090424/GATA3 (−6266), TAF3 (+229728); chr10:8093238-8093283/GATA3 (−3406); chr10:8093464-809352
2/GATA3 (−3174); chr10:8093825-8093859/GATA3 (−2825); chr10:8094258-8094327/GATA3 (−2374); chr10:809
5459-8095492/GATA3 (−1191); chr10:8097588-8098210/GATA3 (+1232); chr10:10840500-10840554/CELF2
(−219366); chr10:11207430-11207709/CELF2 (+147677), USP6NL (+366704); chr10:11207879-11207900/CELF
2 (+147997), USP6NL (+366384); chr10:11784232-11784285/ECHDC3 (−97); chr10:11784386-11784469/ECHD
C3 (+72); chr10:11784702-11784798/ECHDC3 (+394); chr10:12084984-12085023/UPF2 (−164); chr10:1304313
5-13043175/CCDC3 (+549); chr10:13142141-13142186/OPTN (+82); chr10:13701349-13701372/PRPF18 (+72
422), FRMD4A (+671505); chr10:13933134-13933246/PRPF18 (+304251), FRMD4A (+439676); chr10:139335
39-13933596/PRPF18 (+304629), FRMD4A (+439298); chr10:13933935-13933982/PRPF18 (+305020), FRMD
4A (+438907); chr10:13934126-13934168/PRPF18 (+305208), FRMD4A (+438719); chr10:15411721-15411790/
NMT2 (−201061), FAM171A1 (+1302); chr10:16562065-16562344/C1QL3 (+1799), PTER (+83238); chr10:165
62673-16562709/C1QL3 (+1313), PTER (+83724); chr10:16563665-16563690/C1QL3 (+326); chr10:16859907-
16859950/RSU1 (−476); chr10:17243725-17243744/TRDMT1 (+335); chr10:17271255-17271443/VIM (+1091),
ST8SIA6 (+224905); chr10:17495446-17495474/ST8SIA6 (+794); chr10:18429149-18429506/CACNB2 (−278);
chr10:18430151-18430183/CACNB2 (+561); chr10:21789415-21789424/NEBL (−602889), C10orf140 (+17331);
chr10:21822885-21823064/MLLT10 (−599); chr10:22540707-22540740/DNAJC1 (−248074), COMMD3-BMI1 (−6
4588); chr10:22542317-22542461/DNAJC1 (−249739), COMMD3-BMI1 (−62923); chr10:22624306-22624561/S
PAG6 (−9940), COMMD3-BMI1 (+19122); chr10:22625121-22625382/SPAG6 (−9122), COMMD3-BMI1 (+19940);
chr10:22625783-22625811/SPAG6 (−8577), COMMD3-BMI1 (+20485); chr10:22634244-22634309/SPAG6 (−9
7); chr10:22634655-22634820/SPAG6 (+364); chr10:22765792-22765820/SPAG6 (+131432), PIP4K2A (+2376
97); chr10:22766145-22766187/SPAG6 (+131792), PIP4K2A (+237337); chr10:23462463-23462485/PTF1A (−1
8986), MSRB2 (+78047); chr10:23462615-23462634/PTF1A (−18835), MSRB2 (+78198); chr10:23463074-2346
3266/PTF1A (−18290), MSRB2 (+78743); chr10:23463854-23463887/PTF1A (−17589), MSRB2 (+79444); chr10:
23480837-23480880/PTF1A (−601); chr10:23481155-23481296/PTF1A (−234); chr10:23482233-23482288/PTF
1A (+801); chr10:23488259-23488342/PTF1A (+6841); chr10:23632618-23632710/KIAA1217 (−865056), PTF1
A (+151204); chr10:23632941-23632977/KIAA1217 (−864761), PTF1A (+151499); chr10:23633507-23633573/K
IAA1217 (−864180), PTF1A (+152080); chr10:23983342-23983617/KIAA1217 (−514240), PTF1A (+502020); chr
10:23984775-23984841/KIAA1217 (−512912), PTF1A (+503348); chr10:25012963-25012980/ARHGAP21 (−375);
chr10:26223265-26223329/MYO3A (+295); chr10:26500871-26501026/GAD2 (−4287); chr10:26504144-26504
190/GAD2 (−1069); chr10:26504884-26505008/GAD2 (−290); chr10:26505236-26505441/GAD2 (+103); chr10:2
6505618-26505713/GAD2 (+430); chr10:26506619-26506902/APBB1IP (−220505), GAD2 (+1525); chr10:26680
734-26680792/APBB1IP (−46503), GAD2 (+175527); chr10:26727817-26727867/APBB1IP (+576); chr10:27389
391-27389428/ANKRD26 (+17); chr10:27528965-27528976/ACBD5 (+837); chr10:27529714-27529787/ACBD5
(+57); chr10:27531195-27531276/ACBD5 (−1428); chr10:27548332-27548400/ACBD5 (−18558), PTCHD3 (+15
4931); chr10:27608909-27608972/ACBD5 (−79133), PTCHD3 (+94356); chr10:27609162-27609225/ACBD5 (−7
9386), PTCHD3 (+94103); chr10:28034187-28034326/MKX (+521); chr10:28034615-28034756/MKX (+92); chr
10:28287318-28287365/ARMC4 (+635); chr10:28591417-28591456/WAC (−230984), MPP7 (−20370); chr10:28
822687-28822703/WAC (+274); chr10:28822932-28822977/WAC (+534); chr10:28957995-28958043/BAMBI
(−8405), WAC (+135598); chr10:30024657-30024793/SVIL (−100824), MTPAP (+613542); chr10:30316932-30316
987/SVIL (−393059), MTPAP (+321307); chr10:30347783-30347861/SVIL (−423921), MTPAP (+290445); chr10:
30722902-30722934/MAP3K8 (−32); chr10:30996298-30996387/AK302694 (+15140), ZNF438 (+324523); chr1
0:31607868-31607924/ZEB1 (−205); chr10:32049241-32049296/ARHGAP12 (+168501), ZEB1 (+441168); chr1
0:35896911-35896969/GJD4 (+2602), FZD8 (+33422); chr10:35928449-35928530/FZD8 (+1872), GJD4 (+3415
2); chr10:35928871-35929022/FZD8 (+1415), GJD4 (+34609); chr10:38299502-38299539/ZNF33A (−57); chr10:
38383694-38383837/ZNF37A (+502); chr10:38692070-38692155/HSD17B7P2 (+46805); chr10:42673383-4267
3405/ZNF33B (+460598); chr10:42971158-42971325/ZNF33B (+162750); chr10:42971459-42971517/ZNF33B
(+162504); chr10:43048231-43048268/ZNF33B (+85742); chr10:43048649-43048677/ZNF33B (+85329); chr10:
43250119-43250136/ZNF33B (−116136), BMS1 (−27826); chr10:43250780-43250854/ZNF33B (−116825), BMS1
(−27137); chr10:43278289-43278341/BMS1 (+361); chr10:43332632-43332713/RET (−239844), BMS1 (+54719);
chr10:43393643-43393982/RET (−178704), BMS1 (+115859); chr10:43428577-43428636/RET (−143910), BM
S1 (+150653); chr10:43428977-43429066/RET (−143495), BMS1 (+151068); chr10:43429197-43429215/RET
(−143311), BMS1 (+151252); chr10:43600447-43600505/CSGALNACT2 (−33458), RET (+27959); chr10:4369808
7-43698114/CSGALNACT2 (+64167), RASGEF1A (+64266); chr10:43818536-43818591/RASGEF1A (−56197),
FXYD4 (−48528); chr10:43903616-43903672/HNRNPF (−345); chr10:43904037-43904164/HNRNPF (−802); chr1
0:43932148-43932188/ZNF487P (−406); chr10:44162008-44162100/ZNF32 (−17728), HNRNPA3P1 (+123811);
chr10:44163001-44163135/ZNF32 (−18742), HNRNPA3P1 (+122797); chr10:44185015-44185089/ZNF32 (−407
26), HNRNPA3P1 (+100813); chr10:44500617-44500692/HNRNPA3P1 (−214790), CXCL12 (+379890); chr10:4
4788811-44788848/HNRNPA3P1 (−502965), CXCL12 (+91715); chr10:44789047-44789105/HNRNPA3P1 (−503
211), CXCL12 (+91469); chr10:44880455-44880519/CXCL12 (+58); chr10:44880645-44880673/CXCL12 (−114);
chr10:45914965-45915097/ALOX5 (+45402), MARCH8 (+115788); chr10:46222738-46222757/FAM21C (+100);
chr10:46970241-46970375/SYT15 (+293); chr10:46970484-46970604/SYT15 (+57); chr10:46971016-469711
50/SYT15 (−482); chr10:47083378-47083518/PPYR1 (−86); chr10:48377818-48377873/RBP3 (+13145), ZNF48
8 (+22757); chr10:48438779-48438805/GDF10 (+346); chr10:49732088-49732155/ARHGAP22 (+81016), MAP
K8 (+122467); chr10:49892860-49892942/WDFY4 (−617); chr10:50323687-50323824/VSTM4 (−197); chr10:508
17873-50818152/CHAT (−4070), SLC18A3 (−334); chr10:50818632-50818986/CHAT (−3274), SLC18A3 (+462);
chr10:50819408-50819747/CHAT (−2505); chr10:50887754-50887789/CHAT (+65689), OGDHL (+82653); chr1
0:50976615-50977033/OGDHL (−6399), AGAP8 (+269711); chr10:51489157-51489187/AGAP7 (−2845); chr10:
51784604-51784643/FAM21A (−43060), AGAP6 (+36546); chr10:51827748-51827794/FAM21A (+87); chr10:52
177879-52177951/ASAH2 (−169545), SGMS1 (+205822); chr10:53459262-53459294/CSTF2T (+77); chr10:600
26257-60026303/CISD1 (−2615), IPMK (+1414); chr10:60027022-60027085/CISD1 (−1841), IPMK (+640); chr10:
60145595-60145665/TFAM (+454); chr10:60272834-60272895/BICC1 (−39); chr10:60273279-60273346/BICC1
(+409); chr10:60936770-60936950/PHYHIPL (+512); chr10:62492030-62492156/ANK3 (−342459), CDK1 (−461
19); chr10:63422670-63422824/ARID5B (−238266), TMEM26 (−209539); chr10:64575068-64575127/EGR2 (+10
28), ADO (+10582); chr10:64578629-64578651/EGR2 (−2514); chr10:65281121-65281155/REEP3 (+15); chr10:
65801470-65801523/REEP3 (+520374); chr10:69645049-69645081/SIRT1 (+638); chr10:70091423-70091480/
HNRNPH3 (−316); chr10:70231552-70231629/DNA2 (+288); chr10:71329002-71329076/NEUROG3 (+4171), T
SPAN15 (+117813); chr10:71331905-71331949/NEUROG3 (+1283), TSPAN15 (+120701); chr10:71337713-71
337726/NEUROG3 (−4510); chr10:71562837-71562857/H2AFY2 (−249510), COL13A1 (+1203); chr10:7157748
7-71577569/H2AFY2 (−234829), COL13A1 (+15884); chr10:71596714-71596784/H2AFY2 (−215608), COL13A1
(+35105); chr10:71892101-71892126/AIFM2 (+576); chr10:71930093-71930114/SAR1A (+181); chr10:7193051
6-71930538/SAR1A (−242); chr10:72043453-72043778/NPFFR1 (−166); chr10:72200065-72200117/NODAL (+1
374), EIF4EBP2 (+36230); chr10:72200772-72200824/NODAL (+667); chr10:72201740-72201830/NODAL (−32
0); chr10:72218899-72218970/NODAL (−17470), PRF1 (+143596); chr10:72432630-72432660/ADAMTS14 (+86);
chr10:73156216-73156346/CDH23 (−410); chr10:73156868-73156912/CDH23 (+199); chr10:73490213-73490
263/C10orf105 (−10660), C10orf54 (+43099); chr10:73847807-73847817/SPOCK2 (+719); chr10:74057662-740
57707/DDIT4 (+24008), DNAJB12 (+57222); chr10:74079693-74079752/DNAJB12 (+35184), DDIT4 (+46046);
chr10:74385796-74385874/MICU1 (+114); chr10:74927622-74927672/ECD (+206); chr10:75255602-75255635/
PPP3CB (+163); chr10:75385578-75385619/USP54 (−50166), MYOZ1 (+15916); chr10:75407413-75407477/M
YOZ1 (−5930), SYNPO2L (+8387); chr10:75532518-75532564/FUT11 (+492); chr10:75633695-75633736/CAM
K2G (+633); chr10:76871155-76871196/DUSP13 (−2206), SAMD8 (−217); chr10:76993921-76993940/COMTD1
(+1839), VDAC2 (+24019); chr10:76994591-76994649/COMTD1 (+1150), VDAC2 (+24708); chr10:77155392-7
7155469/COMTD1 (−159661), ZNF503 (+6082); chr10:77155701-77155769/COMTD1 (−159965), ZNF503 (+577
8); chr10:77156017-77156165/COMTD1 (−160321), ZNF503 (+5422); chr10:77164970-77165030/ZNF503 (−348
7); chr10:77167346-77167438/ZNF503 (−5879); chr10:77167671-77167874/ZNF503 (−6260); chr10:79975649-7
9975697/ZMIZ1 (−853119), RPS24 (+182155); chr10:80853984-80854038/PPIF (−253209), ZMIZ1 (+25219); chr
10:81002067-81002167/PPIF (−105103), ZMIZ1 (+173325); chr10:81002329-81002562/PPIF (−104774), ZMIZ1
(+173654); chr10:81002925-81002985/PPIF (−104265), ZMIZ1 (+174163); chr10:81026274-81026379/PPIF (−80
893), ZMIZ1 (+197535); chr10:81053854-81053962/PPIF (−53312), ZMIZ1 (+225116); chr10:81163278-811633
37/ZCCHC24 (+40620), PPIF (+56088); chr10:81205737-81205809/ZCCHC24 (−1845), ZCCHC24 (−390); chr10:
81743127-81743191/FAM213A (−95267), SFTPD (−34298); chr10:81904148-81904219/PLAC9 (+11926), ANX
A11 (+61144); chr10:81964769-81964830/ANXA11 (+528); chr10:82116250-82116381/MAT1A (−66882), C10or
f58 (−57259); chr10:82214592-82214651/TSPAN14 (+584); chr10:83634362-83634466/NRG3 (−656); chr10:836
34759-83634796/NRG3 (−292); chr10:83635499-83635530/NRG3 (+445); chr10:83635633-83635670/NRG3
(+582); chr10:85997132-85997241/LRIT2 (−11903), LRIT1 (+4030); chr10:86300864-86300937/RGR (+296092); c
hr10:88122930-88122961/GRID1 (+3304); chr10:88123191-88123616/GRID1 (+2846); chr10:88124646-88124
762/GRID1 (+1546); chr10:88127203-88127270/GRID1 (−987); chr10:88161212-88161255/GRID1 (−34984), W
APAL (+120307); chr10:88281078-88281102/WAPAL (+451); chr10:88295629-88295680/OPN4 (−118659), WA
PAL (−14114); chr10:88296076-88296312/OPN4 (−118120), WAPAL (−14653); chr10:88296557-88296712/OPN
4 (−117679), WAPAL (−15094); chr10:88438958-88439022/BMPR1A (−77406), LDB3 (+10564); chr10:88515778-
88515848/BMPR1A (−583); chr10:88730582-88730629/AL157440 (−35), AGAP11 (+108); chr10:88731541-887
31612/AL157440 (−1006); chr10:88854483-88854570/GLUD1 (+249); chr10:89201423-89201524/GLUD1 (−346
698), MINPP1 (−62749); chr10:90750248-90750398/FAS (+35); chr10:91294013-91294097/SLC16A12 (+1258),
IFIT5 (+119730); chr10:91295492-91295584/SLC16A12 (−225); chr10:91295879-91295923/SLC16A12 (−588);
chr10:92616683-92616758/HTR7 (+950); chr10:92616995-92617016/HTR7 (+665); chr10:93668144-93668182/
FGFBP3 (+1095), TNKS2 (+110012); chr10:94448755-94448826/HHEX (−890); chr10:94448969-94449138/HH
EX (−627); chr10:94451646-94451695/EXOC6 (−156554), HHEX (+1990); chr10:94451826-94451973/EXOC6
(−156325), HHEX (+2219); chr10:94608199-94608294/EXOC6 (+22); chr10:94822415-94822849/CYP26A1 (−110
15), CYP26C1 (+1611); chr10:94823198-94823250/CYP26A1 (−10423), CYP26C1 (+2203); chr10:94825696-94
825982/CYP26A1 (−7808), CYP26C1 (+4818); chr10:94828125-94828193/CYP26A1 (−5488), CYP26C1 (+7138);
chr10:94831930-94831980/CYP26A1 (−1692); chr10:94835224-94835334/CYP26A1 (+1632), MYOF (+406795);
chr10:95008835-95008945/CYP26A1 (+175243), MYOF (+233184); chr10:95462353-95462392/FRA10AC1
(−44); chr10:98946067-98946094/SLIT1 (−398); chr10:98956267-98956313/SLIT1 (−10607), ARHGAP19 (+96140);
chr10:99080931-99080989/FRAT1 (+1938), FRAT2 (+13498); chr10:99178897-99178950/RRP12 (−17797), P
GAM1 (−7103); chr10:99392819-99392886/PI4K2A (−7590), C10orf62 (+43403); chr10:99789970-99790021/CR
TAC1 (+589); chr10:99790340-99790437/CRTAC1 (+196); chr10:99790760-99790917/CRTAC1 (−254); chr10:9
9798264-99798349/C10orf28 (−96074), CRTAC1 (−7722); chr10:100992191-100992221/HPS1 (−785502), HPSE
2 (+3426); chr10:100992647-100992681/HPS1 (−785960), HPSE2 (+2968); chr10:101089532-101089565/CNN
M1 (+693); chr10:101287510-101287571/GOT1 (−97011), NKX2-3 (−5149); chr10:101290161-101290179/NKX2-
3 (−2520); chr10:101291143-101291230/NKX2-3 (−1503); chr10:101296717-101296737/NKX2-3 (+4037), SLC2
5A28 (+83494); chr10:101989305-101989346/CHUK (+18); chr10:102027241-102027268/CWF19L1 (+182); chr
10:102132876-102132949/WNT8B (−89899), SCD (+26141); chr10:102322420-102322508/PAX2 (−183004), HI
F1AN (+26823); chr10:102414938-102415092/PAX2 (−90453), HIF1AN (+119374); chr10:102419267-10241939
9/PAX2 (−86135), HIF1AN (+123692); chr10:102470277-102470339/PAX2 (−35160), HIF1AN (+174667); chr10:
102473716-102473759/PAX2 (−31730), HIF1AN (+178097); chr10:102475471-102475538/PAX2 (−29963), HIF1
AN (+179864); chr10:102484246-102484269/PAX2 (−21210), HIF1AN (+188617); chr10:102489664-102489700/
PAX2 (−15786), HIF1AN (+194041); chr10:102497176-102497252/PAX2 (−8254), HIF1AN (+201573); chr10:10
2505849-102505873/PAX2 (+393); chr10:102507584-102507610/SEMA4G (−224689), PAX2 (+2129); chr10:10
2508290-102508341/SEMA4G (−223970), PAX2 (+2848); chr10:102589186-102589221/SEMA4G (−143082), P
AX2 (+83736); chr10:102833305-102833359/TLX1 (−57729), KAZALD1 (+12333); chr10:102881113-102881240/
TLX1 (−9884), KAZALD1 (+60178); chr10:102891956-102892090/TLX1 (+962); chr10:102892601-102892649/T
LX1 (+1564), LBX1 (+96092); chr10:102893952-102894090/TLX1 (+2960), LBX1 (+94696); chr10:102899856-1
02899948/TLX1 (+8841), LBX1 (+88815); chr10:102900159-102900201/TLX1 (+9119), LBX1 (+88537); chr10:1
02906470-102906524/TLX1 (+15436), LBX1 (+82220); chr10:102983057-102983087/LBX1 (+5645), TLX1 (+92
011); chr10:102983380-102983434/LBX1 (+5310), TLX1 (+92346); chr10:102984371-102984512/LBX1 (+4275),
TLX1 (+93381); chr10:102987020-102987127/LBX1 (+1643), TLX1 (+96013); chr10:102996481-102996596/B
TRC (−117286), LBX1 (−7822); chr10:102996827-102996899/BTRC (−116962), LBX1 (−8146); chr10:102997228-
102997281/BTRC (−116570), LBX1 (−8538); chr10:103043865-103043924/BTRC (−69930), LBX1 (−55178); chr1
0:103044228-103044300/BTRC (−69561), LBX1 (−55547); chr10:103070291-103070305/LBX1 (−81581), BTRC
(−43527); chr10:103454822-103454841/FBXW4 (−89); chr10:103534419-103534562/FBXW4 (−79748), FGF8 (+1
268); chr10:103535771-103535841/FGF8 (−47); chr10:103536257-103536299/FGF8 (−519); chr10:103543029-1
03543067/NPM3 (+122); chr10:103590537-103590543/MGEA5 (−12318), KCNIP2 (+13137); chr10:103601187-
103601231/MGEA5 (−22987), KCNIP2 (+2468); chr10:103603284-103603320/KCNIP2 (+375); chr10:10382510
1-103825150/HPS6 (+2); chr10:104000419-104000613/GBF1 (−4739), PITX3 (+715); chr10:104170263-104170
407/PSD (+8566), NFKB2 (+15996); chr10:104170801-104170870/PSD (+8065), NFKB2 (+16497); chr10:1041
74647-104174683/FBXL15 (−4906), PSD (+4236); chr10:104953006-104953067/NT5C2 (+26); chr10:10503665
9-105036700/INA (−240); chr10:105036883-105036893/INA (−32); chr10:105037892-105037955/INA (+1004), P
CGF6 (+72967); chr10:105244551-105244660/NEURL (−9129), CALHM3 (−5609); chr10:105253828-105253853/
NEURL (+106); chr10:105318861-105318973/NEURL (+65182), SH3PXD2A (+296247); chr10:105344252-105
344401/NEURL (+90592), SH3PXD2A (+270837); chr10:105344545-105344797/NEURL (+90936), SH3PXD2A
(+270493); chr10:105726496-105726544/SLK (−950); chr10:106097959-106098025/ITPRIP (−4329); chr10:1061
13446-106113480/SORCS3 (−287396), ITPRIP (−19800); chr10:106398688-106398825/SORCS3 (−2102); chr10:
106401029-106401111/SORCS3 (+211); chr10:106401302-106401321/SORCS3 (+453); chr10:106401433-10
6401510/SORCS3 (+613); chr10:106402191-106402271/SORCS3 (+1372); chr10:108923898-108923945/SOR
CS1 (+544); chr10:108924060-108924345/SORCS1 (+263); chr10:109674208-109674377/SORCS1 (−749827);
chr10:109674559-109674853/SORCS1 (−750240); chr10:110226169-110226435/NONE; chr10:110671884-110
671929/NONE; chr10:111216573-111216610/XPNPEP1 (+466719); chr10:111216804-111217161/XPNPEP1
(+466328); chr10:111971017-111971053/MXI1 (+3672), SMNDC1 (+93672); chr10:112257163-112257205/DUSP
5 (−441); chr10:112258656-112258702/SMC3 (−68770), DUSP5 (+1054); chr10:112838355-112838551/ADRA2
A (+1663); chr10:114206724-114206783/ZDHHC6 (−82), VTI1A (−2); chr10:114207016-114207054/ZDHHC6 (−3
63), VTI1A (+279); chr10:115439571-115439598/CASP7 (+159); chr10:115804562-115804635/ADRB1 (+793);
chr10:116853622-116853672/ATRNL1 (+523); chr10:118030706-118031061/GFRA1 (+2242); chr10:11803154
9-118031624/GFRA1 (+1539); chr10:118032230-118032412/GFRA1 (+805); chr10:118033994-118034142/GF
RA1 (−942); chr10:118368789-118368847/PNLIPRP2 (−11647), PNLIPRP1 (+18328); chr10:118369578-118369
604/PNLIPRP2 (−10874), PNLIPRP1 (+19101); chr10:118429552-118429771/PNLIPRP2 (+49197), HSPA12A
(+72423); chr10:118501942-118501983/HSPA12A (+122); chr10:118891134-118891179/KIAA1598 (−126069), V
AX1 (+6655); chr10:118891662-118891715/KIAA1598 (−126601), VAX1 (+6123); chr10:118892457-118892517/
KIAA1598 (−127399), VAX1 (+5325); chr10:118893581-118893679/KIAA1598 (−128542), VAX1 (+4182); chr10:
118893892-118894034/KIAA1598 (−128875), VAX1 (+3849); chr10:118894309-118894371/KIAA1598 (−129252),
VAX1 (+3472); chr10:118897419-118897478/VAX1 (+363); chr10:118899394-118899582/VAX1 (−1676); chr10:
118899790-118899879/VAX1 (−2023); chr10:118900245-118900323/VAX1 (−2472); chr10:118922924-1189229
43/KCNK18 (−34066), VAX1 (−25122); chr10:118927963-118927999/VAX1 (−30169), KCNK18 (−29019); chr10:1
18976401-118976444/SLC18A2 (−24161), KCNK18 (+19423); chr10:118976550-118976625/SLC18A2 (−23996),
KCNK18 (+19588); chr10:119000592-119000689/SLC18A2 (+57); chr10:119001155-119001328/SLC18A2 (+6
58); chr10:119292253-119292323/PDZD8 (−157351), EMX2 (−9668); chr10:119294898-119294908/PDZD8 (−15
9966), EMX2 (−7053); chr10:119295909-119295951/PDZD8 (−160993), EMX2 (−6026); chr10:119296431-11929
6524/PDZD8 (−161541), EMX2 (−5478); chr10:119296644-119296755/PDZD8 (−161763), EMX2 (−5256); chr10:1
19301428-119301759/EMX2 (−362); chr10:119302156-119302221/EMX2 (+233); chr10:119304479-119304501/
EMX2 (+2534), RAB11FIP2 (+501624); chr10:119304829-119304850/EMX2 (+2884), RAB11FIP2 (+501274); c
hr10:119306187-119306273/EMX2 (+4274), RAB11FIP2 (+499884); chr10:119310375-119310433/EMX2 (+844
8), RAB11FIP2 (+495710); chr10:119313504-119313528/EMX2 (+11560), RAB11FIP2 (+492598); chr10:11949
4597-119494848/EMX2 (+192767), RAB11FIP2 (+311391); chr10:120354330-120354368/PRLHR (+811); chr10:
120354736-120354843/PRLHR (+370); chr10:120355426-120355461/PRLHR (−284); chr10:120788981-12078
9021/NANOS1 (−227); chr10:120938355-120938396/PRDX3 (−31); chr10:121301184-121301363/RGS10 (+948);
chr10:121355815-121355859/TIAL1 (+704); chr10:121578082-121578116/MCMBP (+54295), INPP5F (+9254
0); chr10:121652425-121652472/SEC23IP (+364); chr10:121733837-121733935/PPAPDC1A (−482580), SEC2
3IP (+81801); chr10:121887594-121887641/PPAPDC1A (−328848), SEC23IP (+235533); chr10:122216757-122
216793/PPAPDC1A (+309); chr10:123688098-123688166/ATE1 (−586); chr10:123922683-123922724/PLEKHA
1 (−211390), TACC2 (+174015); chr10:123922862-123922903/PLEKHA1 (−211211), TACC2 (+174194); chr10:1
23923255-123923517/PLEKHA1 (−210708), TACC2 (+174697); chr10:123923796-123923880/PLEKHA1 (−2102
56), TACC2 (+175149); chr10:124060800-124060867/PLEKHA1 (−73260), TACC2 (+312145); chr10:124221065-
124221086/HTRA1 (+35); chr10:124638990-124639033/FAM24B (+145); chr10:124767931-124767994/ACAD
SB (−466), IKZF5 (+348); chr10:124893193-124893237/HMX3 (−2352); chr10:124895696-124895832/HMX3 (+1
97); chr10:124896712-124896744/HMX2 (−10910), HMX3 (+1161); chr10:124902047-124902138/HMX2 (−5545),
HMX3 (+6526); chr10:124902569-124902607/HMX2 (−5050), HMX3 (+7021); chr10:124905880-124905919/H
MX2 (−1738); chr10:124910455-124910708/BUB3 (−3178); chr10:125425612-125425689/GPR26 (−220); chr10:1
25425948-125426031/GPR26 (+119); chr10:125651167-125651293/CPXM2 (+270); chr10:125732479-125732
508/CPXM2 (−80994), CHST15 (+119436); chr10:125852498-125852540/CHST15 (−589); chr10:126135831-12
6135877/OAT (−28309), NKX1-2 (+2696); chr10:126222979-126222995/LHPP (+72646), FAM53B (+209943); c
hr10:126223218-126223244/LHPP (+72890), FAM53B (+209699); chr10:126329353-126329392/FAM53B (+10
3557), LHPP (+179032); chr10:126330564-126330614/FAM53B (+102341), LHPP (+180248); chr10:126398485-
126398628/FAM53B (+34373), LHPP (+248216); chr10:126480174-126480210/METTL10 (+247); chr10:12648
0360-126480377/METTL10 (+70); chr10:126605509-126605545/ZRANB1 (−25165), FAM175B (+115173); chr1
0:126606121-126606169/ZRANB1 (−24547), FAM175B (+115791); chr10:126840891-126840952/C10orf137 (−5
67162), CTBP2 (−124469); chr10:126847357-126847410/C10orf137 (−560700), CTBP2 (−130931); chr10:12684
7869-126847900/C10orf137 (−560199), CTBP2 (−131432); chr10:126850792-126850824/C10orf137 (−557276),
CTBP2 (−134355); chr10:127512510-127512531/UROS (−684), BCCIP (+417); chr10:128076863-128076940/A
DAM12 (+225); chr10:128077295-128077416/ADAM12 (−229); chr10:128993522-128993580/NPS (−354062), D
OCK1 (+399528); chr10:128994529-128994545/NPS (−353076), DOCK1 (+400514); chr10:129534586-1295349
92/FOXI2 (−749); chr10:129535447-129535695/FOXI2 (+33); chr10:129536224-129536258/FOXI2 (+703); chr1
0:129845665-129845735/MKI67 (+78768), PTPRE (+140375); chr10:129924518-129924546/MKI67 (−64); chr10:
130008877-130008906/MKI67 (−84424); chr10:130009288-130009346/MKI67 (−84849); chr10:130010133-1300
10211/MKI67 (−85704); chr10:130064059-130064183/MKI67 (−139653); chr10:130085276-130085355/MKI67
(−160848); chr10:130338552-130338585/MGMT (−926885), MKI67 (−414101); chr10:130338769-130338803/MG
MT (−926668), MKI67 (−414318); chr10:130508450-130508472/MGMT (−756993), MKI67 (−583993); chr10:1305
08595-130508634/MGMT (−756839), MKI67 (−584147); chr10:131502608-131502726/MGMT (+237213), EBF3
(+259424); chr10:131694768-131694792/EBF3 (+67311), MGMT (+429326); chr10:131757096-131757545/EBF
3 (+4770), MGMT (+491867); chr10:131757853-131758276/EBF3 (+4026), MGMT (+492611); chr10:13176341
6-131763611/EBF3 (−1423); chr10:131765744-131765970/EBF3 (−3766); chr10:131766125-131766190/EBF3
(−4067); chr10:131766321-131766558/EBF3 (−4349); chr10:131766757-131766862/EBF3 (−4719); chr10:131767
504-131767542/GLRX3 (−167116), EBF3 (−5432); chr10:131767676-131767751/GLRX3 (−166925), EBF3 (−562
3); chr10:131768028-131768057/GLRX3 (−166596), EBF3 (−5952); chr10:131771094-131771191/GLRX3 (−1634
96), EBF3 (−9052); chr10:131771302-131771415/GLRX3 (−163280), EBF3 (−9268); chr10:133109787-13310993
8/PPP2R2D (−638097); chr10:133110351-133110553/PPP2R2D (−637508); chr10:133110875-133110966/PPP2
R2D (−637039); chr10:133795242-133795312/BNIP3 (+158); chr10:133795884-133795975/BNIP3 (−495); chr10:
133796102-133796163/BNIP3 (−698); chr10:133850135-133850156/JAKMIP3 (−68167), BNIP3 (−54711); chr10:
133946714-133946772/DPYSL4 (−53671), JAKMIP3 (+28430); chr10:133999523-133999554/DPYSL4 (−875); c
hr10:133999687-133999776/DPYSL4 (−682); chr10:134000053-134000108/DPYSL4 (−333); chr10:134000426-1
34000529/DPYSL4 (+64); chr10:134000740-134000798/DPYSL4 (+355); chr10:134000948-134001139/DPYSL
4 (+630); chr10:134045869-134045912/DPYSL4 (+45477), STK32C (+75586); chr10:134110221-134110325/S
TK32C (+11204), DPYSL4 (+109859); chr10:134145271-134145293/INPP5A (−206071), STK32C (−23805); chr1
0:134234819-134234906/INPP5A (−116490), STK32C (−113386); chr10:134235679-134235720/INPP5A (−1156
53), STK32C (−114223); chr10:134358081-134358179/INPP5A (+6777), NKX6-2 (+241407); chr10:134388556-
134388646/INPP5A (+37248), NKX6-2 (+210936); chr10:134441735-134441808/INPP5A (+90419), NKX6-2 (+1
57765); chr10:134597647-134597971/NKX6-2 (+1728), INPP5A (+246456); chr10:134598125-134598164/NKX
6-2 (+1392), INPP5A (+246792); chr10:134598534-134598827/NKX6-2 (+856); chr10:134600017-134600037/N
KX6-2 (−490); chr10:134721478-134721649/NKX6-2 (−122027), TTC40 (+34525); chr10:134736329-134736443/
NKX6-2 (−136849), TTC40 (+19703); chr10:134755722-134755772/TTC40 (+342); chr10:134756383-13475640
2/TTC40 (−304); chr10:134827245-134827266/TTC40 (−71167), GPR123 (−57177); chr10:134843686-13484375
6/TTC40 (−87632), GPR123 (−40712); chr10:134902125-134902187/KNDC1 (−71815), GPR123 (+17723); chr10:
134936166-134936331/KNDC1 (−37722), GPR123 (+51816); chr10:134974272-134974307/KNDC1 (+319); chr
10:135040117-135040173/UTF1 (−3633); chr10:135043626-135043826/UTF1 (−52); chr10:135044391-1350445
54/UTF1 (+695); chr10:135071063-135071117/ADAM8 (+19317), VENTX (+19682); chr10:135092461-1350925
57/ADAM8 (−2102); chr10:135121663-135121725/ZNF511 (−729), TUBGCP2 (+950); chr10:135132162-135132
200/TUBGCP2 (−9537), CALY (+18294); chr10:135149222-135149265/TUBGCP2 (−26600), CALY (+1231); chr
10:135187009-135187027/ECHS1 (−110); chr10:135498960-135498995/DUX4L3 (+8699); chr11:207196-20723
2/RIC8A (−1316), BET1L (+208); chr11:208402-208468/BET1L (−1013), RIC8A (−95); chr11:279569-279619/AT
HL1 (−9544), NLRP6 (+1024); chr11:289112-289126/ATHL1 (−19); chr11:308166-308230/IFITM2 (+91); chr11:3
26834-326944/B4GALNT4 (−42906), IFITM3 (−5975); chr11:415429-415440/SIGIRR (+1962), PKP3 (+21218); c
hr11:416498-416565/SIGIRR (+865); chr11:503351-503443/RNH1 (+3424), PTDSS2 (+53117); chr11:535449-5
35466/HRAS (+92); chr11:560721-560737/RASSF7 (−721); chr11:576712-576759/PHRF1 (+253); chr11:615650-
615793/IRF7 (+277); chr11:626878-627094/CDHR5 (−1919), SCT (+187); chr11:627391-627433/CDHR5 (−234
5), SCT (−239); chr11:636947-637161/DRD4 (−251); chr11:637264-637349/DRD4 (+2); chr11:637601-637831/D
RD4 (+411); chr11:639574-639667/TMEM80 (−55995), DRD4 (+2316); chr11:640166-640245/TMEM80 (−55410),
DRD4 (+2901); chr11:694996-695035/TMEM80 (−600), DEAF1 (+724); chr11:720572-720696/TALDO1 (−2679
8), EPS8L2 (+14514); chr11:720892-720933/TALDO1 (−26519), EPS8L2 (+14793); chr11:721855-721951/TALD
O1 (−25529), EPS8L2 (+15783); chr11:722122-722134/TALDO1 (−25304), EPS8L2 (+16008); chr11:725638-725
812/TALDO1 (−21707), EPS8L2 (+19605); chr11:726035-726086/TALDO1 (−21371), EPS8L2 (+19941); chr11:7
47092-747122/TALDO1 (−325); chr11:787766-787883/PDDC1 (−10338), CEND1 (+2301); chr11:798453-798514/
SLC25A22 (−215); chr11:805428-805498/RPLP2 (−4473), PIDD (−213); chr11:809906-809954/PIDD (−4680), RP
LP2 (−6); chr11:829049-829095/CD151 (−3880); chr11:830244-830288/CD151 (−2686); chr11:830589-830701/C
D151 (−2307); chr11:911465-911511/CHID1 (−614); chr11:1241180-1241271/MUC5B (−3069); chr11:1319584-1
319647/TOLLIP (+11276), MUC5B (+75321); chr11:1330300-1330356/TOLLIP (+564); chr11:1330466-1330505/
TOLLIP (+406); chr11:1358566-1358609/BRSK2 (−52541), TOLLIP (−27696); chr11:1411534-1411549/BRSK2
(+413); chr11:1457141-1457207/MOB2 (+44941), BRSK2 (+46045); chr11:1470184-1470345/MOB2 (+31850), B
RSK2 (+59136); chr11:1577990-1578005/MOB2 (−70050), DUSP8 (+15152); chr11:1769585-1769644/IFITM10
(+2209), KRTAP5-6 (+51190); chr11:1769864-1770262/IFITM10 (+1761), KRTAP5-6 (+51638); chr11:1779778-
1779926/IFITM10 (−8028), CTSD (+5370); chr11:1780073-1780166/IFITM10 (−8296), CTSD (+5102); chr11:185
9277-1859348/TNNI2 (−2119); chr11:1874265-1874441/LSP1 (−12042), TNNI2 (+12921); chr11:1910501-19107
88/TNNT3 (−30154), LSP1 (+24250); chr11:2121673-2121752/IGF2 (+40628), MRPL23 (+153211); chr11:21594
26-2159472/IGF2 (+2892), MRPL23 (+190947); chr11:2161694-2161724/IGF2 (+632); chr11:2162113-2162141/
IGF2 (+214); chr11:2162482-2162544/IGF2 (−172); chr11:2165215-2165258/IGF2 (−2896); chr11:2165651-216
5670/IGF2 (−3320); chr11:2291018-2291072/TH (−98010), ASCL2 (+1137); chr11:2291925-2291944/ASCL2 (+2
47); chr11:2292058-2292105/ASCL2 (+100); chr11:2292360-2292391/ASCL2 (−194); chr11:2399323-2399399/
CD81 (+814); chr11:2465448-2465461/KCNQ1 (−766); chr11:2466176-2466191/KCNQ1 (−37); chr11:2469053-2
469137/KCNQ1 (+2874), CDKN1C (+437900); chr11:2488692-2488846/KCNQ1 (+22548), CDKN1C (+418226);
chr11:2801638-2801667/CDKN1C (+105342), KCNQ1 (+335432); chr11:2803036-2803087/CDKN1C (+103933),
KCNQ1 (+336841); chr11:2812959-2813023/CDKN1C (+94004), KCNQ1 (+346770); chr11:2813194-2813247/
CDKN1C (+93774), KCNQ1 (+347000); chr11:2859573-2859581/CDKN1C (+47418), KCNQ1 (+393356); chr11:
2859783-2859898/CDKN1C (+47154), KCNQ1 (+393620); chr11:2884144-2884158/CDKN1C (+22844), KCNQ
1 (+417930); chr11:2891061-2891233/CDKN1C (+15848), KCNQ1 (+424926); chr11:2923650-2923698/SLC22
A18AS (+1501), SLC22A18 (+2723); chr11:2935498-2935531/SLC22A18AS (−10340), PHLDA2 (+15135); chr1
1:3148344-3148451/CARS (−69717), OSBPL5 (+38184); chr11:3150355-3150434/CARS (−71714), OSBPL5 (+3
6187); chr11:4116137-4116183/RRM1 (+236); chr11:4233393-4233448/RRM1 (+117497), OR52B4 (+156195);
chr11:6502385-6502438/FXC1 (−265), ARFIP2 (+297); chr11:6633503-6633548/TAF10 (−51); chr11:6676553-6
676627/DCHS1 (+484); chr11:6676968-6677036/DCHS1 (+72); chr11:6704160-6704215/MRPL17 (+444); chr1
1:7272929-7273066/SYT9 (−183); chr11:7273260-7273679/SYT9 (+289); chr117273899-7274075/SYT9 (+806);
chr11:8040564-8040581/TUB (−19607), EIF3F (+32128); chr11:8040776-8040960/TUB (−19312), EIF3F (+324
23); chr11:8102326-8102368/TUB (+42167), RIC3 (+88243); chr11:8290021-8290057/LMO1 (−4633); chr11:898
5909-8985976/TMEM9B (+46); chr11:9112586-9112639/SCUBE2 (+537); chr11:9685180-9685218/SWAP70
(−429); chr11:10315719-10315760/SBF2 (+14); chr11:10326448-10326499/ADM (−168); chr11:10472522-104725
57/AMPD3 (+316); chr11:10715174-10715216/CTR9 (−57616), MRVI1 (−41347); chr11:10879328-10879371/ZB
ED5 (+270); chr11:12246230-12246347/MICALCL (−62158), MICAL2 (+114151); chr11:12695496-12695572/TE
AD1 (−435); chr11:13032760-13032789/ARNTL (−266550), RASSF10 (+1805); chr11:14379771-14379827/RRA
S2 (+931); chr11:14379984-14380040/RRAS2 (+718); chr11:14521424-14521484/COPB1 (−39); chr11:1466594
9-14665995/PDE3B (+703); chr11:14926601-14926712/CYP2R1 (−12906), CALCA (+67175); chr11:14926945-
14926975/CYP2R1 (−13209), CALCA (+66872); chr11:14927226-14927337/CYP2R1 (−13531), CALCA (+66550);
chr11:14995163-14995208/CALCA (−1354); chr11:14995865-14995927/CALCA (−2064); chr11:15095203-150
95368/CALCB (+140); chr11:15962840-15962947/SOX6 (+461519), INSC (+828924); chr11:16632081-166320
99/SOX6 (−207677), C11orf58 (−128058); chr11:16632428-16632513/SOX6 (−208058), C11orf58 (−127677); chr
11:16760177-16760216/C11orf58 (+49); chr11:17099311-17099363/RPS13 (−117); chr11:17229380-17229437/
NUCB2 (−68877), PIK3C2A (−38056); chr11:17373571-17373642/B7H6 (+298); chr11:17497521-17497545/ABC
C8 (+916); chr11:17741268-17741528/MYOD1 (+288); chr11:17741890-17741952/MYOD1 (+811); chr11:1774
2484-17742518/KCNC1 (−14994), MYOD1 (+1391); chr11:17756972-17756993/KCNC1 (−512); chr11:17757341-
17757671/KCNC1 (+11); chr11:17758069-17758241/KCNC1 (+660); chr11:18034806-18034821/SERGEF (−17
7); chr11:18230753-18230802/MRGPRX4 (+36394), SAA2-SAA4 (+39443); chr11:18813543-18813609/PTPN5
(−187); chr11:19139102-19139147/ZDHHC13 (+433); chr11:19261980-19262049/E2F8 (+492); chr11:19263268-
19263323/E2F8 (−789); chr11:19264056-19264093/E2F8 (−1568); chr11:19367135-19367267/NAV2 (−367680),
E2F8 (−104694); chr11:19367977-19368011/NAV2 (−366887), E2F8 (−105487); chr11:20177843-20178093/DB
X1 (+3902), NAV2 (+443087); chr11:20181644-20181724/DBX1 (+186); chr11:20182234-20182258/DBX1 (−376);
chr11:20385718-20385788/HTATIP2 (+464); chr11:20617871-20617973/SLC6A5 (−3024); chr11:20618393-2
0618422/SLC6A5 (−2538); chr11:20618925-20619219/SLC6A5 (−1874); chr11:20622793-20623258/NELL1 (−68
091), SLC6A5 (+2080); chr11:20627061-20627348/NELL1 (−63912), SLC6A5 (+6259); chr11:20690878-206909
82/NELL1 (−187); chr11:20691086-20691121/NELL1 (−13); chr11:20691380-20691431/NELL1 (+289); chr11:20
691616-20691747/NELL1 (+565); chr11:22359418-22359485/SLC17A6 (−215); chr11:22454259-22454378/SLC
17A6 (+94652), FANCF (+193068); chr11:22646720-22646772/FANCF (+641); chr11:22850779-22850850/SVI
P (+567); chr11:24518454-24518529/LUZP2 (−24); chr11:24518646-24518677/LUZP2 (+146); chr11:27740208-
27740255/BDNF (−17632), KIF18A (+389514); chr11:30037752-30037883/KCNA4 (+759); chr11:30606161-306
06215/MPPED2 (−4147); chr11:30606755-30606795/MPPED2 (−4734); chr11:31818513-31818570/PAX6 (+209
67), ELP4 (+287245); chr11:31820361-31820460/PAX6 (+19098), ELP4 (+289114); chr11:31825755-31825832/
PAX6 (+13715), ELP4 (+294497); chr11:31826071-31826106/PAX6 (+13420), ELP4 (+294792); chr11:318263
04-31826408/PAX6 (+13153), ELP4 (+295059); chr11:31826733-31826839/PAX6 (+12723), ELP4 (+295489); c
hr11:31827520-31827597/PAX6 (+11950), ELP4 (+296262); chr11:31837513-31837541/PAX6 (+1982), ELP4
(+306230); chr11:31839946-31840041/PAX6 (−485); chr11:31840559-31840602/PAX6 (−1072); chr11:31840711-
31840768/PAX6 (−1231); chr11:31842089-31842174/PAX6 (−2623); chr11:31847302-31847370/RCN1 (−265141),
PAX6 (−7827); chr11:31847713-31847769/RCN1 (−264736), PAX6 (−8232); chr11:31847873-31847895/RCN1
(−264593), PAX6 (−8375); chr11:31848603-31848722/RCN1 (−263814), PAX6 (−9154); chr11:32008882-320090
00/PAX6 (−169432), RCN1 (−103536); chr11:32009127-32009269/PAX6 (−169689), RCN1 (−103279); chr11:323
54960-32354999/WT1 (+102101), RCN1 (+242503); chr11:32355364-32355482/WT1 (+101658), RCN1 (+2429
46); chr11:32448894-32449104/WT1 (+8082), RCN1 (+336522); chr11:32452337-32452424/WT1 (+4700), RCN
1 (+339904); chr11:32452596-32452623/WT1 (+4471), RCN1 (+340133); chr11:32453234-32453253/WT1 (+38
37), RCN1 (+340767); chr11:32454918-32455062/WT1 (+2091), RCN1 (+342513); chr11:32456446-32456758/
WT1 (+479); chr11:32457199-32457219/WT1 (−128); chr11:32459451-32459576/WT1 (−2433); chr11:32460362-
32460464/WT1 (−3332); chr11:32460586-32460659/WT1 (−3542); chr11:32460908-32460979/WT1 (−3863); chr
11:32461092-32461162/WT1 (−4046); chr11:32605267-32605306/EIF3M (−104); chr11:33721944-33721966/00
59 (+22318), C11orf41 (+158078); chr11:33722568-33722597/CD59 (+21690), C11orf41 (+158706); chr11:337
96476-33796530/FBXO3 (−432); chr11:35439822-35439896/SLC1A2 (+1246), CD44 (+279442); chr11:3544009
8-35440137/SLC1A2 (+987); chr11:35440375-35440469/SLC1A2 (+683); chr11:35440885-35440936/SLC1A2
(+194); chr11:40315421-40315465/LRRC4C (+221); chr11:43380430-43380483/TTC17 (+22); chr11:43569075-
43569128/HSD17B12 (−133041), TTC17 (+188667); chr11:43603037-43603076/HSD17B12 (−99086), TTC17
(+222622); chr11:43604146-43604240/HSD17B12 (−97950), TTC17 (+223758); chr11:43604880-43604980/HSD1
7B12 (−97213), TTC17 (+224495); chr11:43902111-43902161/ALKBH3 (−221); chr11:44324975-44325391/ALX
4 (+6533), EXT2 (+207436); chr11:44325770-44325886/ALX4 (+5888), EXT2 (+208081); chr11:44326350-4432
6515/ALX4 (+5283), EXT2 (+208686); chr11:44330609-44330877/ALX4 (+973); chr11:44331440-44331482/AL
X4 (+255); chr11:44333462-44333476/ALX4 (−1753); chr11:44337629-44337726/CD82 (−249463), ALX4 (−5962);
chr11:44337862-44337882/CD82 (−249269), ALX4 (−6156); chr11:44338058-44338086/CD82 (−249069), ALX
4 (−6356); chr11:45306995-45307046/SYT13 (+863); chr11:45377078-45377121/SYT13 (−69216), CHST1 (+31
0106); chr11:45686424-45686449/CHST1 (+769); chr11:45907494-45907521/MAPK8IP1 (+461); chr11:461411
71-46141229/PHF21A (+1785), GYLTL1B (+198004); chr11:46316833-46316895/DGKZ (−66281), CREB3L1
(+17636); chr11:46317356-46317407/DGKZ (−65763), CREB3L1 (+18154); chr11:46354566-46354603/DGKZ (−2
8560), CREB3L1 (+55357); chr11:46412240-46412276/CHRM4 (−4151); chr11:46414368-46414468/CHRM4 (−6
311), AMBRA1 (+198496); chr11:46615625-46615662/AMBRA1 (−2730); chr11:46722959-46723021/ARHGAP1
(−870), ZNF408 (+673); chr11:46848187-46848210/CKAP5 (+19660), F2 (+107456); chr11:46939498-4693952
6/LRP4 (+661); chr11:47197640-47197710/LRP4 (−257502), ARFGAP2 (+1001); chr11:47198877-47198934/AR
FGAP2 (−230); chr11:47207899-47207922/PACSIN3 (+99); chr11:47416419-47416534/SPI1 (−16350), SLC39A
13 (−13569); chr11:47429895-47429925/SLC39A13 (−136); chr11:47430274-47430322/SLC39A13 (+252); chr1
1:47574517-47574565/CELF1 (−63965), PTPMT1 (−12441); chr11:47609824-47609860/FAM180B (+1612), C1
QTNF4 (+6119); chr11:47788925-47788955/AGBL2 (−52012), NUP160 (+81117); chr11:49229665-49229730/F
OLH1 (+524); chr11:49230035-49230099/FOLH1 (+155); chr11:55640410-55640448/SPRYD5 (−10344), OR5D
16 (+34201); chr11:57103297-57103333/P2RX3 (−2526), SSRP1 (+36); chr11:57227222-57227249/RTN4RL2
(−1103); chr11:57244189-57244209/RTN4RL2 (+15860), SLC43A1 (+38159); chr11:57244437-57244461/RTN4R
L2 (+16110), SLC43A1 (+37909); chr11:57250545-57250757/RTN4RL2 (+22312), SLC43A1 (+31707); chr11:5
7283277-57283303/SLC43A1 (−932); chr11:57414622-57414662/YPEL4 (+2775), SERPING1 (+48937); chr11:5
8345196-58345264/LPXN (−1840), ZFP91 (−1357); chr11:58345757-58345802/LPXN (−2390), ZFP91 (−807); chr
11:58874089-58874208/FAM111B (−509); chr11:58874385-58874476/FAM111B (−227); chr11:58903530-58903
571/FAM111A (−8701), FAM111B (+28893); chr11:58940607-58940666/DTX4 (+825); chr11:60609731-606097
73/CCDC86 (+323); chr11:60718854-60718976/SLC15A3 (+342); chr11:60775048-60775226/CD5 (−94793), C
D6 (+36024); chr11:60779665-60779738/CD5 (−90228), CD6 (+40589); chr11:61063024-61063061/VWCE (−25
5); chr11:61276504-61276535/SYT7 (+71824), SDHAF2 (+78923); chr11:61277128-61277153/SYT7 (+71203),
SDHAF2 (+79544); chr11:61322826-61322921/SYT7 (+25470), SDHAF2 (+125277); chr11:61452161-6145225
6/C11orf9 (−67912), DAGLA (+4304); chr11:61509972-61510080/C11orf9 (−10095), DAGLA (+62121); chr11:61
544844-61544889/C11orf10 (+15218), C11orf9 (+24746); chr11:61582699-61582756/FADS1 (+1801), FEN1
(+22619); chr11:61685037-61685087/RAB3IL1 (−65); chr11:61722987-61723400/BEST1 (+5838), FTH1 (+11938);
chr11:61880060-61880122/FTH1 (−144959), INCENP (−11354); chr11:61891685-61891713/INCENP (+254); c
hr11:62212011-62212174/SCGB1A1 (+25586), AHNAK (+102239); chr11:62359505-62359564/TUT1 (−426); ch
r11:62368870-62368902/MTA2 (+426); chr11:62380155-62380206/ROM1 (−32), EML3 (+56); chr11:62433123-
62433206/METTL12 (+386), METTL12 (+386); chr11:62455529-62455590/UBXN1 (−9033), LRRN4CL (+1811);
chr11:62477028-62477103/BSCL2 (−1887); chr11:62494180-62494223/TTC9C (−1750), HNRNPUL2 (+655); chr
11:62521370-62521417/ZBTB3 (+262); chr11:62539040-62539068/TAF6L (+279); chr11:62554212-62554242/
TMEM179B (−647); chr11:62554566-62554597/TMEM179B (−292); chr11:62608916-62608984/WDR74 (−1322);
chr11:62693057-62693135/CHRM1 (−4084); chr11:62693511-62693633/CHRM1 (−4560); chr11:62694083-6269
4283/CHRM1 (−5171), SLC22A6 (+58312); chr11:62694517-62694555/CHRM1 (−5524), SLC22A6 (+57959); chr
11:62694689-62694722/CHRM1 (−5694), SLC22A6 (+57789); chr11:62695679-62695756/CHRM1 (−6706), SLC
22A6 (+56777); chr11:63535939-63535977/C11orf95 (+155); chr11:63637232-63637403/MARK2 (+30918), RC
OR2 (+46998); chr11:63741979-63742038/COX8A (−70); chr11:63766830-63766917/FLRT1 (−104488), OTUB1
(+13549); chr11:63767029-63767245/FLRT1 (−104225), OTUB1 (+13812); chr11:63767448-63767518/FLRT1
(−103879), OTUB1 (+14158); chr11:63767885-63767925/FLRT1 (−103457), OTUB1 (+14580); chr11:63768436-6
3768619/FLRT1 (−102834), OTUB1 (+15203); chr11:63775190-63775230/FLRT1 (−96152), OTUB1 (+21885); c
hr11:63775400-63775531/FLRT1 (−95896), OTUB1 (+22141); chr11:63827740-63827861/FLRT1 (−43561), OT
UB1 (+74476); chr11:63828105-63828189/FLRT1 (−43215), OTUB1 (+74822); chr11:63828882-63828957/FLR
T1 (−42442), OTUB1 (+75595); chr11:63913806-63913901/MACROD1 (+19731), FLRT1 (+42492); chr11:63933
131-63933186/MACROD1 (+426); chr11:63933789-63933827/MACROD1 (−223); chr11:63972858-63972948/F
ERMT3 (−1249); chr11:63974393-63974539/FERMT3 (+314); chr11:64002334-64002368/VEGFB (+295); chr11:
64018526-64018569/PPP1R14B (−4135), PLCB3 (−447); chr11:64018686-64018726/PPP1R14B (−4293), PLCB
3 (−289); chr11:64060111-64060160/C11orf20 (−6682), KCNK4 (+1343); chr11:64064621-64064662/C11orf20
(−2176); chr11:64068021-64068047/ESRRA (−5010), C11orf20 (+1216); chr11:64108101-64108286/CCDC88B
(+504); chr11:64410185-64410203/SLC22A12 (+51912), NRXN2 (+80466); chr11:64480331-64480379/NRXN2
(+10305), SLC22A12 (+122073); chr11:64490258-64490299/NRXN2 (+381); chr11:64490807-64490825/NRXN2
(−156); chr11:64512180-64512196/RASGRP2 (+141); chr11:64577597-64577695/MEN1 (+542); chr11:6461878
1-64618887/CDC42BPG (−6793), EHD1 (+27357); chr11:64655726-64655823/EHD1 (−9584), ATG2A (+28947);
chr11:64660890-64660995/EHD1 (−14752), ATG2A (+23779); chr11:64815215-64815241/SAC3D1 (+6852), NA
ALADL1 (+10781); chr11:64851366-64851416/ZFPL1 (−303), CDCA5 (+224); chr11:64880411-64880471/BC10
4003 (−3434); chr11:65145929-65145991/SLC25A45 (+4182), TIGD3 (+23678); chr11:65313893-65314121/LTB
P3 (+11692), SCYL1 (+21459); chr11:65314382-65314432/LTBP3 (+11292), SCYL1 (+21859); chr11:65325124-
65325157/LTBP3 (+558); chr11:65339897-65339923/EHBP1L1 (−3599); chr11:65341515-65341594/EHBP1L1
(−1954); chr11:65408406-65408616/SIPA1 (+2933), RELA (+21932); chr11:65479379-65479399/KAT5 (−84); ch
r11:65585786-65585810/SNX32 (−15612), OVOL1 (+31293); chr11:65661532-65661563/CCDC85B (+3673), F
OSL1 (+6449); chr11:65769350-65769416/BANF1 (−167), EIF1AD (+254); chr11:65816520-65816560/SF3B2
(−3276), GAL3ST3 (+111); chr11:65838144-65838192/PACS1 (+344); chr11:66080699-66080764/CD248 (+3783),
TMEM151A (+21359); chr11:66103563-66103585/RIN1 (+426); chr11:66188485-66188570/NPAS4 (+53); chr
11:66188784-66188852/NPAS4 (+343); chr11:66206131-66206159/MRPL11 (+165); chr11:66313902-6631395
7/ACTN3 (−461), ZDHHC24 (−259); chr11:66327033-66327088/CTSF (+8986), ACTN3 (+12670); chr11:6640599
0-66406041/RBM14 (+21963), RBM4B (+39259); chr11:66624921-66624983/LRFN4 (+76); chr11:66791118-66
791246/SYT12 (+366); chr11:66824097-66824137/RHOD (−172); chr11:66839186-66839279/KDM2A (−47507),
RHOD (+14944); chr11:67034421-67034446/ADRBK1 (+529); chr11:67042267-67042318/SSH3 (−28626), ADR
BK1 (+8388); chr11:67070454-67070525/SSH3 (−429); chr11:67139461-67139534/POLD4 (−18446), CLCF1 (+2
150); chr11:67140120-67140146/POLD4 (−19081), CLCF1 (+1515); chr11:67140683-67140720/CLCF1 (+946);
chr11:67164664-67164734/PPP1CA (+4677), RAD9A (+5276); chr11:67171082-67171144/PPP1CA (−1737), TB
C1D10C (−299); chr11:67176853-67177119/CARNS1 (−6163), TBC1D10C (+5574); chr11:67232402-67232464/
TMEM134 (+4298), CABP4 (+9615); chr11:67250459-67250527/AIP (−12); chr11:67414419-67414476/1TBX10
(−7417), ACY3 (+3682); chr11:67479283-67479341/ALDH3B2 (−30627), UNC93B1 (+292281); chr11:67723262-6
7723287/ALDH3B2 (−274590), UNC93B1 (+48318); chr11:67770815-67770864/UNC93B1 (+753); chr11:67913
681-67913821/CHKA (−24893), SUV420H1 (+67033); chr11:67980996-67981026/SUV420H1 (−227); chr11:680
49737-68049808/LRP5 (−30335), C11orf24 (−10304); chr11:68079704-68079753/LRP5 (−379); chr11:68080705-
68080734/LRP5 (+612); chr11:68141702-68141741/PPP6R3 (−86464), LRP5 (+61614); chr11:68502784-68502
899/MTL5 (+16146), GAL (+50859); chr11:68596663-68596777/MTL5 (−77732), CPT1A (+12679); chr11:68611
560-68611600/CPT1A (−2181); chr11:68611729-68611752/CPT1A (−2342); chr11:68671146-68671203/IGHMB
P2 (−144), MRPL21 (+128); chr11:68847883-68848039/CCND1 (−607912), TPCN2 (+31611); chr11:69080670-6
9080724/CCND1 (−375176), TPCN2 (+264347); chr11:69189420-69189539/CCND1 (−266393), TPCN2 (+37313
0); chr11:69451377-69451404/CCND1 (−4482); chr11:69541215-69541289/FGF19 (−22146), FGF4 (+48919); c
hr11:69632196-69632221/FGF4 (−42038), FGF3 (+1983); chr11:69633741-69633750/FGF3 (+446); chr11:6963
4490-69634535/FGF3 (−321); chr11:69683377-69683413/ANO1 (−241013), FGF3 (−49203); chr11:69830518-69
830624/FGF3 (−196379), ANO1 (−93837); chr11:69832191-69832231/FGF3 (−198019), ANO1 (−92197); chr11:6
9920053-69920113/ANO1 (−4325); chr11:70117428-70117464/PPFIA1 (+640); chr11:70256925-70257034/CTT
N (+12368), SHANK2 (+678862); chr11:70270228-70270264/CTTN (+25634), SHANK2 (+665596); chr11:7027
8185-70278250/CTTN (+33606), SHANK2 (+657624); chr11:70374809-70374852/CTTN (+130219), SHANK2
(+561011); chr11:70413082-70413190/CTTN (+168524), SHANK2 (+522706); chr11:70508058-70508179/CTTN
(+263507), SHANK2 (+427723); chr11:70735555-70735623/SHANK2 (+200253), CTTN (+490977); chr11:7096
1699-70961871/SHANK2 (−25943), DHCR7 (+197692); chr11:71159389-71159453/NADSYN1 (−4796), DHCR7
(+56); chr11:71318810-71318952/DEFB108B (−225365), KRTAP5-11 (−24960); chr11:71497528-71497580/KRT
AP5-11 (−203633), DEFB108B (−46692); chr11:71524539-71524581/KRTAP5-11 (−230639), DEFB108B (−19686);
chr11:71951375-71951406/PHOX2A (+3829), INPPL1 (+15509); chr11:71952417-71952458/PHOX2A (+2782),
INPPL1 (+16556); chr11:71954824-71955006/PHOX2A (+305); chr11:71955125-71955189/PHOX2A (+63); c
hr11:71955519-71955549/PHOX2A (−314); chr11:72295437-72295544/CLPB (−149923), PDE2A (+90006); chr1
1:72301583-72301704/CLPB (−156076), PDE2A (+83853); chr11:72525873-72525937/ATG16L2 (+454); chr11:
72536189-72536283/ATG16L2 (+10785), FCHSD2 (+316907); chr1172975427-72975490/P2RY6 (−7788), P2R
Y2 (+46115); chr11:73471760-73471818/RAB6A (+412); chr11:73471971-73472038/RAB6A (+196); chr11:736
68838-73668924/DNAJB13 (+7517), UCP2 (+25008); chr11:73881799-73881871/PPME1 (−533), C2CD3 (+229);
chr11:74178406-74178470/KCNE3 (+162); chr11:74204140-74204173/MGC12965 (−328), LIPT2 (+598); chr1
1:74303493-74303538/POLD3 (−113); chr11:74442643-74442705/CHRDL2 (−488); chr11:74952930-74952948/
SLCO2B1 (+90907), ARRB1 (+109936); chr11:74953337-74953489/SLCO2B1 (+91381), ARRB1 (+109462); ch
r11:75095656-75095705/ARRB1 (−32806), RPS3 (−14881); chr11:75110549-75110581/RPS3 (+3); chr11:75140
878-75140939/RPS3 (+30347), GDPD5 (+95690); chr11:75236234-75236270/GDPD5 (+347); chr11:75294622-
75294662/SERPINH1 (+21541), MAP6 (+84837); chr11:75295538-75295594/SERPINH1 (+22465), MAP6 (+83
913); chr11:75379249-75379282/MAP6 (+213); chr11:75946679-75946766/WNT11 (−29149), PRKRIR (+14515
7); chr11:75947182-75947207/WNT11 (−29621), PRKRIR (+144685); chr11:76033291-76033342/WNT11 (−115
743), PRKRIR (+58563); chr11:76750933-76751048/CAPN5 (−27001), B3GNT6 (+5556); chr11:79152475-7915
2508/ODZ4 (−797); chr11:82443569-82443651/PRCP (+167947); chr11:82611623-82611666/C11orf82 (−1092),
PRCP (−88); chr11:82782250-82782314/RAB30 (+602); chr11:82996865-82996920/CCDC90B (+484); chr11:82
997285-82997368/CCDC90B (+50); chr11:86085862-86085931/C11orf73 (+72644), ME3 (+297343); chr11:879
08819-87908907/RAB38 (−228); chr11:88241976-88242130/CTSC (−171112), GRM5 (+539187); chr11:8824227
5-88242358/CTSC (−171376), GRM5 (+538923); chr11:88242624-88242705/CTSC (−171724), GRM5 (+538575);
chr11:88796788-88796810/TYR (−114241), GRM5 (−15559); chr11:88798931-88798965/TYR (−112092), GRM
5 (−17708); chr11:89224663-89224735/NOX4 (−46); chr11:89520697-89520729/TRIM49 (+21030), FOLH1B (+1
28248); chr11:89597088-89597136/LOC399940 (+11102), TRIM64B (+12073); chr11:89597496-89597571/LOC
399940 (+11524), TRIM64B (+11651); chr11:89597727-89597773/TRIM64B (+11435), LOC399940 (+11740); c
hr11:89785245-89785281/UBTFL1 (−33855), TRIM49L2 (+20989); chr11:89785646-89785710/UBTFL1 (−33440),
TRIM49L2 (+21404); chr11:91959327-91959354/FAT3 (−125921); chr11:92702848-92703034/MTNR1B (+152);
chr11:92930635-92930674/SLC36A4 (+440); chr11:93275775-93275864/C11orf75 (+726); chr11:93474620-9
3474649/C11orf54 (−158), TAF1D (+68); chr11:94134541-94134682/GPR83 (−27); chr11:94277098-94277135/F
UT4 (+100); chr11:94473769-94473802/AMOTL1 (−27722), PIWIL4 (+173312); chr11:94502058-94502100/AM
OTL1 (+571); chr11:94502552-94502757/AMOTL1 (+1147), CWC15 (+204121); chr11:94706511-94706543/KD
M4D (−318), CWC15 (+249); chr11:94706954-94706995/CWC15 (−199), KDM4D (+130); chr11:94771035-9477
1070/SRSF8 (−29003), KDM4DL (+12631); chr11:94883729-94883964/ENDOD1 (+60873), SESN3 (+80399); c
hr11:95070805-95070895/CEP57 (−452775), SESN3 (−106604); chr11:95522853-95522871/CEP57 (−763); chr1
1:95523950-95524001/CEP57 (+351); chr11:97616832-97616881/NONE; chr11:100999041-100999159/PGR
(+1444), TMEM133 (+136289); chr11:101453538-101453715/PGR (−453083), TRPC6 (+1032); chr11:10145409
3-101454170/TRPC6 (+527); chr11:101454362-101454496/TRPC6 (+230); chr11:105480787-105480858/GRIA
4 (+23); chr11:105948097-105948131/AASDHPPT (−178); chr11:106889317-106889400/GUCY1A2 (−188); chr1
1:107047948-107048033/GUCY1A2 (−158820), CWF19L2 (+280581); chr11:107328480-107328524/CWF19L2
(+70); chr11:107436098-107436157/ALKBH8 (+333); chr11:107436441-107436492/ALKBH8 (−6); chr11:108093
337-108093385/ATM (−198), NPAT (+4); chr11:109292830-109292891/C11orf87 (+15); chr11:109293764-1092
93873/C11orf87 (+973); chr11:109964620-109964697/ZC3H12C (+572); chr11:110301145-110301186/FDX1
(+505); chr11:110583029-110583043/ARHGAP20 (+415); chr11:111093701-111093794/ARHGAP20 (−510297), P
OU2AF1 (+156409); chr11:111169445-111169472/ARHGAP20 (−586008), POU2AF1 (+80698); chr11:1113075
06-111307554/POU2AF1 (−57373), BTG4 (+75549); chr11:111383532-111383557/BTG4 (−466); chr11:1114115
82-111411821/LAYN (+469); chr11:111749895-111749928/C11orf1 (−36), FDXACB1 (+269); chr11:111783479-
111783503/CRYAB (−1018), HSPB2 (+31); chr11:111847783-111847801/DIXDC1 (−241); chr11:111957072-111
957125/SDHD (−472), TIMM8B (+423); chr11:112832712-112832736/NCAM1 (+755); chr11:112834035-112834
116/TTC12 (−351175), NCAM1 (+2107); chr11:112834332-112834375/TTC12 (−350897), NCAM1 (+2385); chr1
1:113745967-113746039/USP28 (+253); chr11:113846911-113846936/ZBTB16 (−84364), HTR3A (+1127); chr1
1:113906881-113906924/ZBTB16 (−24385), HTR3A (+61106); chr11:113945421-113945435/NNMT (−221107),
ZBTB16 (+14140); chr11:114271473-114271531/RBM7 (+118); chr11:115501225-115501342/CADM1 (−126043);
chr11:115516380-115516438/CADM1 (−141168); chr11:115530096-115530221/CADM1 (−154918); chr11:115
531261-115531319/CADM1 (−156049); chr11:115630474-115630530/CADM1 (−255261); chr11:116033699-116
033793/CADM1 (−658505), ZNF259 (+624993); chr11:116371462-116371503/CADM1 (−996242), ZNF259 (+28
7256); chr11:116371851-116371911/CADM1 (−996640), ZNF259 (+286858); chr11:116451953-116452029/ZNF
259 (+206748); chr11:116658755-116658826/ZNF259 (−52); chr11:116662044-116662075/ZNF259 (−3321); chr
11:116968703-116968714/SIK3 (+284); chr11:117015348-117015390/PAFAH1B2 (+369); chr11:117102773-11
7102827/RNF214 (−604), PCSK7 (+11); chr11:117314711-117314745/CEP164 (+116157), DSCAML1 (+353248);
chr11:117666712-117666733/DSCAML1 (+1253), CEP164 (+468152); chr11:117694418-117694451/DSCAM
L1 (−26459), FXYD6-FXYD2 (+53311); chr11:118051251-118051352/SCN2B (−3965); chr11:118272061-118272
137/ATP5L (−5); chr11:118480274-118480317/ARCN1 (+37194), TREH (+70085); chr11:118481040-11848110
1/ARCN1 (+37969), TREH (+69310); chr11:118927811-118927867/HYOU1 (+86); chr11:118965117-11896517
8/H2AFX (+1029), HMBS (+9561); chr11:118972050-118972130/DPAGT1 (+695); chr11:118978008-11897803
1/C2CD2L (−73); chr11:119020623-119020671/ABCG4 (+897); chr11:119187226-119187246/MCAM (+604); ch
r11:119205423-119205436/RNF26 (+220); chr11:119234391-119234482/C1QTNF5 (−17054), USP2 (+17999);
chr11:119293320-119293352/THY1 (+910); chr11:119598945-119599011/PVRL1 (+457); chr11:119612268-11
9612323/PVRL1 (−12861), TRIM29 (+396567); chr11:119664280-119664443/PVRL1 (−64927), TRIM29 (+34450
1); chr11:120381284-120381335/GRIK4 (−149668), ARHGEF12 (+173692); chr11:120383016-120383054/GRIK
4 (−147943), ARHGEF12 (+175417); chr11:120434822-120434954/GRIK4 (−96090), ARHGEF12 (+227270); chr
11:121349766-121349930/SORL1 (+26936), BLID (+637075); chr11:122848313-122848368/BSX (+4038), CRT
AM (+139086); chr11:122850124-122850148/BSX (+2243), CRTAM (+140881); chr11:122855666-122855721/B
SX (−3315); chr11:122932232-122932297/HSPA8 (+636); chr11:123172863-123172915/GRAMD1B (−223639),
CLMP (−106882); chr11:123228716-123228788/GRAMD1B (−167776), CLMP (−162745); chr11:123229407-123
229461/GRAMD1B (−167094), CLMP (−163427); chr11:123301029-123301082/CLMP (−235049), GRAMD1B (−9
5472); chr11:123947079-123947220/OLFR959 (−81975), OR10G7 (−37442); chr11:124629419-124629499/VSI
G2 (−7350), ESAM (+2764); chr11:124630372-124630410/VSIG2 (−8282), ESAM (+1832); chr11:124664934-12
4665009/ROBO3 (−70333), ESAM (−32749); chr11:124709192-124709233/ESAM (−76990), ROBO3 (−26092); c
hr11:124735352-124735374/ROBO3 (+58); chr11:124747533-124747587/ROBO3 (+12255), ROBO4 (+20271);
chr11:124790854-124791118/HEPN1 (+1840), HEPACAM (+15322); chr11:124823983-124824011/CCDC15
(−20); chr11:125036337-125036497/PKNOX2 (+1858), FEZ1 (+329789); chr11:125495290-125495337/CHEK1
(−937); chr11:125774061-125774122/PUS3 (−976), DDX25 (−180); chr11:125774449-125774480/PUS3 (−1349), D
DX25 (+193); chr11:125932550-125932556/CDON (+634); chr11:126081180-126081235/RPUSD4 (+379); chr1
1:126081947-126082002/RPUSD4 (−388); chr11:126138825-126138866/FOXRED1 (−89), SRPR (+31); chr11:1
26226662-126226677/ST3GAL4 (+1130), KIRREL3 (+644096); chr11:126870501-126870524/KIRREL3 (+253);
chr11:128555224-128555316/ETS1 (−97817), FLI1 (−8541); chr11:128556481-128556532/ETS1 (−99054), FLI1
(−7304); chr11:128563186-128563336/FLI1 (−550); chr11:128564544-128564602/FLI1 (+762); chr11:128564877-
128564991/FLI1 (+1123), KCNJ1 (+147429); chr11:128694457-128694518/KCNJ1 (+17875), FLI1 (+130677); c
hr11:129149040-129149096/BARX2 (−96813), ARHGAP32 (−86975); chr11:129242617-129242675/BARX2 (−32
35); chr11:129243242-129243304/BARX2 (−2608); chr11:129244301-129244440/BARX2 (−1510); chr11:129765
331-129765437/NFRKB (−2480); chr11:129871720-129871764/PRDM10 (+988); chr11:129872116-129872136/
PRDM10 (+604); chr11:129940838-129940854/APLP2 (+383); chr11:131780331-131780535/NTM (−279); chr11:
131780777-131780834/NTM (+94); chr11:131781079-131781281/NTM (+468); chr11:131781587-131781648/
NTM (+906); chr11:132812632-132812674/OPCML (+384); chr11:132812990-132813045/OPCML (+19); chr11:
132813758-132813907/OPCML (−796); chr11:132953051-132953232/OPCML (−140105), SPATA19 (+762250);
chr11:133399768-133399856/OPCML (−586775), SPATA19 (+315580); chr11:133657711-133657734/OPCML
(−844686), SPATA19 (+57669); chr11:133826659-133826702/IGSF9B (+199); chr11:133994758-133994839/JA
M3 (+55979), NCAPD3 (+99627); chr11:134122881-134122924/ACAD8 (−531), THYN1 (+357); chr11:13414642
7-134146444/GLB1L3 (+161); chr11:134147413-134147458/GLB1L2 (−54332), GLB1L3 (+1161); chr11:134281
643-134281706/B3GAT1 (+137); chr11:134429596-134429681/B3GAT1 (−147827); chr12:312984-313001/SLC
6A12 (+10378), IQSEC3 (+136944); chr12:498867-498918/KDM5A (−273); chr12:662872-662960/B4GALNT3
(+93373), NINJ2 (+109839); chr12:861644-861677/WNK1 (−428); chr12:1756220-1756294/ADIPOR2 (−43990), W
NT5B (+17845); chr12:1771071-1771153/ADIPOR2 (−29135), WNT5B (+32700); chr12:1906436-1906476/LRT
M2 (−22977), ADIPOR2 (+106209); chr12:2030166-2030316/CACNA2D4 (−2371); chr12:2113390-2113432/DCP
1B (+266); chr12:2162054-2162067/CACNA1C (−355); chr12:2393190-2393284/FKBP4 (−510871), CACNA1C
(+230821); chr12:2734542-2734590/FKBP4 (−169542), CACNA1C (+572150); chr12:2798315-2798398/FKBP4
(−105751), CACNA1C (+635941); chr12:2800470-2800602/FKBP4 (−103572), CACNA1C (+638120); chr12:2800
908-2800937/FKBP4 (−103185), CACNA1C (+638507); chr12:2862143-2862267/FKBP4 (−41903), CACNA1C
(+699789); chr12:2903415-2903520/FKBP4 (−640); chr12:2905324-2905364/FKBP4 (+1236), NRIP2 (+38877); ch
r12:2986619-2986637/FOXM1 (−307), C12orf32 (+263); chr12:3068517-3068568/TEAD4 (+65); chr12:3123966-
3124000/TSPAN9 (−62538), TEAD4 (+55505); chr12:3239850-3239924/PRMT8 (−360538), TSPAN9 (+53366); c
hr12:3310076-3310150/PRMT8 (−290312), TSPAN9 (+123592); chr12:3600783-3600974/PRMT8 (+454); chr12:
3601342-3601377/PRMT8 (+935); chr12:3602717-3602864/PRMT8 (+2366), EFCAB4B (+259575); chr12:4273
886-4274001/PARP11 (−291336), CCND2 (−108958); chr12:4274420-4274474/PARP11 (−291839), CCND2 (−10
8455); chr12:4671347-4671496/DYRK4 (−27822), RAD51AP1 (+23472); chr12:4918683-4918713/KCNA6 (+356);
chr12:4919214-4919238/KCNA6 (+884); chr12:5019035-5019084/KCNA1 (−13); chr12:5019743-5019793/KC
NA1 (+695); chr12:5020314-5020440/KCNA5 (−132708), KCNA1 (+1304); chr12:5153287-5153357/KCNA5 (+2
37); chr12:5153461-5153656/KCNA5 (+474); chr12:6054168-6054231/ANO2 (+1198), NTF3 (+512920); chr12:
6054583-6054617/ANO2 (+798); chr12:6184466-6184515/ANO2 (−129093), VWF (+49345); chr12:6254615-625
4704/CD9 (−54822), VWF (−20824); chr12:6419566-6419603/PLEKHG6 (−17); chr12:6420169-6420228/PLEKH
G6 (+597); chr12:6427652-6427824/PLEKHG6 (+8136), TNFRSF1A (+23545); chr12:6446758-6446827/TNFRS
F1A (+4490), PLEKHG6 (+27191); chr12:6493449-6493504/LTBR (+120); chr12:6664346-6664370/IFFO1 (+89
1); chr12:6664615-6664702/IFFO1 (+590); chr12:6665276-6665329/IFFO1 (−54); chr12:6665438-6665528/IFFO
1 (−234); chr12:6677269-6677315/NOP2 (+188); chr12:6716398-6716434/CHD4 (+135); chr12:6756535-675664
0/ACRBP (−8); chr12:6772022-6772059/ING4 (+267); chr12:6797845-6797908/ZNF384 (+861); chr12:6875798-
6875827/PTMS (+272); chr12:6876078-6876101/PTMS (+549); chr12:6882912-6883010/CD4 (−15677), LAG3
(+1291); chr12:6883142-6883182/CD4 (−15476), LAG3 (+1492); chr12:7023681-7023730/ENO2 (+92); chr12:70
46237-7046406/PTPN6 (−9418), ATN1 (+12696); chr12:7055991-7056017/PTPN6 (+264); chr12:7061426-7061
574/PTPN6 (+5760), PHB2 (+18416); chr12:7068170-7068289/PHB2 (+11686), PTPN6 (+12490); chr12:71262
11-7126241/LPCAT3 (−384); chr12:7810819-7810948/CD163 (−154470), APOBEC1 (+7618); chr12:8025373-80
25634/AY455283 (−30), SLC2A14 (−9); chr12:8025755-8025860/SLC2A14 (−313), AY455283 (+274); chr12:8234
807-8234840/NECAP1 (+17); chr12:9067354-9067389/PHC1 (+56); chr12:9966761-9966895/CD69 (−53331), K
LRF1 (−13249); chr12:10826531-10826564/STYK1 (+343); chr12:11653464-11653509/ETV6 (−149301), PRB2
(−104989); chr12:11919406-11919510/BCL2L14 (−304420), ETV6 (+116670); chr12:12419920-12419964/LRP6
(−131); chr12:12715034-12715089/DUSP16 (+386); chr12:13069264-13069316/GPRC5A (+25334), GPRC5D
(+34028); chr12:14956430-14956469/WBP11 (−49); chr12:15942738-15942801/EPS8 (−260); chr12:20522232-20
522347/PDE3A (+111); chr12:21927839-21927877/KCNJ8 (−111); chr12:22094269-22094577/ABCC9 (−4795);
chr12:22095048-22095181/CMAS (−104044), ABCC9 (−5487); chr12:22199090-22199123/CMAS (−52); chr12:2
2486882-22487133/ST8SIA1 (+640); chr12:22488267-22488339/ST8SIA1 (−655); chr12:25055261-25055309/B
CAT1 (+724); chr12:25055633-25056242/BCAT1 (+71); chr12:25101998-25102009/LRMP (−103177), BCAT1
(−45995); chr12:25403771-25403796/KRAS (+70); chr12:26111601-26111628/RASSF8 (−349); chr12:26112281-2
6112305/RASSF8 (+329); chr12:26986068-26986098/ITPR2 (+48); chr12:26986920-26986998/ITPR2 (−828); c
hr12:27091135-27091184/FGFR1OP2 (−145), ASUN (+94); chr12:27091904-27091952/ASUN (−674), FGFR1O
P2 (+623); chr12:28127352-28127422/PTHLH (−2471); chr12:29301426-29301524/FAR2 (−75123), CCDC91
(+891340); chr12:29935410-29935454/OVCH1 (−284813), TMTC1 (+1311); chr12:29935839-29935902/TMTC1
(+872); chr12:29936323-29936395/TMTC1 (+384); chr12:29936516-29936542/TMTC1 (+214); chr12:29936643-2
9936661/TMTC1 (+91); chr12:29936832-29936976/TMTC1 (−161); chr12:29937402-29937496/TMTC1 (−706); c
hr12:30322925-30323014/TMTC1 (−386227), IPO8 (+525959); chr12:30323440-30323502/TMTC1 (−386728), I
PO8 (+525458); chr12:30353921-30353956/TMTC1 (−417196), IPO8 (+494990); chr12:30354459-30354599/TM
TC1 (−417786), IPO8 (+494400); chr12:30907149-30907225/CAPRIN2 (+261); chr12:30948977-30948988/TSP
AN11 (−130855), CAPRIN2 (−41535); chr12:30975325-30975388/TSPAN11 (−104481), CAPRIN2 (−67909); chr1
2:30975960-30976290/TSPAN11 (−103713), CAPRIN2 (−68677); chr12:31079368-31079417/TSPAN11 (−445); c
hr12:31079664-31079693/TSPAN11 (−159); chr12:31769423-31769475/METTL20 (−43160), DENND5B (−25497);
chr12:32259591-32259615/BICD1 (−582); chr12:33049624-33049652/PKP2 (+142); chr12:33592013-3359205
7/SYT10 (+719); chr12:33592254-33592284/SYT10 (+485); chr12:33593034-33593092/SYT10 (−309); chr12:34
175261-34175408/ALG10 (+119); chr12:34260841-34260905/ALG10 (+85657); chr12:38710536-38710582/AL
G10B (+2); chr12:39538628-39538688/KIF21A (+298534), ALG10B (+828101); chr12:39538939-39539283/KIF
21A (+298081), ALG10B (+828554); chr12:40501452-40501485/SLC2A13 (−1808); chr12:41086664-41086692/
CNTN1 (+320); chr12:41582207-41582398/PDZRN4 (+53); chr12:42538705-42538731/GXYLT1 (−45); chr12:42
631802-42631879/YAF2 (+313); chr12:42632091-42632177/YAF2 (+20); chr12:42982866-42982950/PRICKLE
1 (−105492), ADAMTS20 (+962816); chr12:43944881-43945109/ADAMTS20 (+729); chr12:43945380-4394541
6/ADAMTS20 (+326); chr12:43945781-43946202/ADAMTS20 (−268); chr12:45269387-45269416/NELL2 (+1231);
chr12:45444682-45444714/DBX2 (+184); chr12:46663633-46663671/SLC38A1 (−444); chr12:46777295-4677
7318/SLC38A2 (−10662), SLC38A4 (+442473); chr12:47224974-47225027/SLC38A4 (−5221), AMIGO2 (+24873
3); chr12:47225303-47225319/SLC38A4 (−5531), AMIGO2 (+248423); chr12:47225477-47225550/SLC38A4 (−5
734), AMIGO2 (+248220); chr12:47473849-47473886/AMIGO2 (−134); chr12:48099605-48099674/RPAP3 (+20
4); chr12:48099791-48099829/RPAP3 (+34); chr12:48134470-48134526/ENDOU (−15143), RAPGEF3 (+18391);
chr12:48213229-48213282/HDAC7 (+507); chr12:48214048-48214120/HDAC7 (−321); chr12:48336118-48336
156/VDR (−37323), TMEM106C (−21193); chr12:48357264-48357313/TMEM106C (−41); chr12:48592634-48592
685/OR10AD1 (+4415), C12orf68 (+15294); chr12:48690881-48690970/OR10AD1 (−93851), H1FNT (−31837); c
hr12:49110381-49110426/CCNT1 (+377); chr12:49211866-49211931/CACNB3 (+3684), DDX23 (+34058); chr1
2:49245891-49245939/DDX23 (+42); chr12:49319010-49319065/FKBP11 (+292); chr12:49350839-49350875/A
RF3 (+395); chr12:49350978-49351119/ARF3 (+203); chr12:49364238-49364415/ARF3 (−13075), WNT10B (+1
314); chr12:49375027-49375115/WNT1 (+2835), DDN (+18017); chr12:49390725-49390823/DDN (+2314), WN
T1 (+18538); chr12:49391105-49391146/DDN (+1962), WNT1 (+18890); chr12:49483554-49483609/RHEBL1
(−19807), DHH (+5020); chr12:49484861-49485104/RHEBL1 (−21208), DHH (+3619); chr12:49488071-49488131/
DHH (+501); chr12:49689097-49689134/PRPH (+207); chr12:49689392-49689466/PRPH (+520); chr12:497174
00-49717475/TROAP (+467); chr12:49726859-49727091/C1QL4 (+3996), TROAP (+10004); chr12:49731617-4
9731707/C1QL4 (−691); chr12:49735769-49735822/C1QL4 (−4825); chr12:49933259-49933289/KCNH3 (+334);
chr12:49942621-49942951/KCNH3 (+9846), MCRS1 (+17436); chr12:49943102-49943165/KCNH3 (+10194),
MCRS1 (+17088); chr12:50101536-50101574/FMNL3 (−358); chr12:50135042-50135166/TMBIM6 (−488); chr12:
50221947-50222003/BCDIN3D (+14937), TMBIM6 (+86383); chr12:50297911-50297952/FAIM2 (−172); chr12:
50349159-50349271/AQP5 (−6064), AQP2 (+4691); chr12:50350298-50350353/AQP5 (−4953); chr12:50505380-
50505542/C12orf62 (−439); chr12:50560393-50560468/CERS5 (+666); chr12:50560806-50560839/CERS5 (+2
74); chr12:51158245-51158271/ATF1 (+469); chr12:51663957-51663987/SMAGP (+230); chr12:51717594-517
17657/BIN2 (+312); chr12:51717855-51717946/BIN2 (+37); chr12:51718087-51718198/BIN2 (−205); chr12:519
85415-51985463/SCN8A (+419); chr12:52115423-52115652/FIGNL2 (+110163), SCN8A (+130518); chr12:522
07965-52208001/FIGNL2 (+17718), SCN8A (+222963); chr12:52214118-52214230/FIGNL2 (+11527), SCN8A
(+229154); chr12:52214427-52214507/FIGNL2 (+11234), SCN8A (+229447); chr12:52214702-52214925/FIGNL
2 (+10887), SCN8A (+229794); chr12:52262682-52262735/ACVRL1 (−43404), FIGNL2 (−37008); chr12:523008
41-52300859/FIGNL2 (−75149), ACVRL1 (−5263); chr12:52301386-52301494/ACVRL1 (−4673); chr12:52400489-
52400516/GRASP (−245); chr12:52401539-52401605/GRASP (+824); chr12:52430580-52430619/NR4A1 (−145
86), GRASP (+29852); chr12:52463301-52463367/C12orf44 (−424); chr12:52626747-52626830/KRT7 (−165); ch
r12:52627102-52627229/KRT7 (+212); chr12:52644187-52644231/AK300121 (+7962), KRT7 (+17255); chr12:5
2652251-52652360/AK300121 (−135); chr12:52652506-52652598/AK300121 (−381); chr12:52994989-52995004/
KRT72 (+325); chr12:52995142-52995207/KRT72 (+147); chr12:53273249-53273300/KRT78 (−30497), KRT8
(+25593); chr12:53359316-53359385/EIF4B (−40711), KRT18 (+16508); chr12:53440704-53440742/TENC1 (−31
12); chr12:53591688-53591720/CSAD (−17011), ITGB7 (+9296); chr12:53626155-53626251/RARG (−163); chr1
2:53718632-53718690/AAAS (−3249); chr12:53738593-53738648/SP1 (−35358), SP7 (−9084); chr12:53845682-
53845741/PCBP2 (−174); chr12:54070771-54070830/ATP5G2 (−692); chr12:54088831-54088951/ATP5G2 (−18
782), CALCOCO1 (+32416); chr12:54090594-54090662/ATP5G2 (−20519), CALCOCO1 (+30679); chr12:54142
073-54142109/HOXC13 (−190485), CALCOCO1 (−20784); chr12:54145858-54145880/HOXC13 (−186707), CAL
COCO1 (−24562); chr12:54329479-54329604/HOXC13 (−3034); chr12:54338818-54338885/HOXC12 (−9862), H
OXC13 (+6276); chr12:54343830-54343968/HOXC12 (−4815); chr12:54345659-54345891/HOXC12 (−2939); chr
12:54349080-54349255/HOXC12 (+454); chr12:54349635-54349715/HOXC12 (+961); chr12:54351325-543513
75/HOXC11 (−15560), HOXC12 (+2636); chr12:54354694-54354814/HOXC11 (−12156), HOXC12 (+6040); chr1
2:54355139-54355184/HOXC11 (−11748), HOXC12 (+6448); chr12:54355399-54355475/HOXC11 (−11473), HO
XC12 (+6723); chr12:54357568-54357603/HOXC11 (−9324), HOXC12 (+8872); chr12:54359683-54359742/HO
XC11 (−7197), HOXC12 (+10999); chr12:54367375-54367429/HOXC11 (+492); chr12:54377947-54377977/HO
XC10 (−984); chr12:54379931-54379958/HOXC10 (+999); chr12:54384665-54384762/HOXC9 (−9163), HOXC1
0 (+5768); chr12:54391324-54391399/HOXC9 (−2515); chr12:54393724-54393798/HOXC9 (−116); chr12:54394
419-54394466/HOXC9 (+566); chr12:54398786-54398888/HOXC8 (−4053); chr12:54409194-54409267/HOXC6
(−12963), HOXC8 (+6341); chr12:54412089-54412129/HOXC6 (−10085), HOXC8 (+9219); chr12:54422346-544
22376/HOXC5 (−4471), HOXC6 (+167); chr12:54423464-54423565/HOXC5 (−3317); chr12:54424749-54425031/
HOXC5 (−1942); chr12:54425141-54425221/HOXC5 (−1651); chr12:54427932-54427974/HOXC4 (−19708), HO
XC5 (+1121); chr12:54428394-54428574/HOXC4 (−19177), HOXC5 (+1652); chr12:54441288-54441391/HOXC
4 (−6321), HOXC5 (+14508); chr12:54441837-54441889/HOXC4 (−5798), HOXC5 (+15031); chr12:54447833-54
447898/HOXC4 (+205); chr12:54484258-54484310/HOXC4 (+36623), SMUG1 (+98494); chr12:54500665-5450
0739/HOXC4 (+53041), SMUG1 (+82076); chr12:54520194-54520221/SMUG1 (+62570), HOXC4 (+72547); chr
12:54552988-54553118/SMUG1 (+29725), HOXC4 (+105392); chr12:54762581-54762616/GPR84 (−4329); chr
12:54811841-54811879/ZNF385A (−26777), ITGA5 (+1190); chr12:56040105-56040133/METTL7B (−35211), O
R10P1 (+9443); chr12:56122228-56122269/CD63 (+661); chr12:56224611-56224653/DNAJC14 (−2134); chr12:
56473818-56473838/ERBB3 (+19); chr12:56473969-56473998/ERBB3 (+175); chr12:56660529-56660589/CO
Q10A (−83); chr12:56728124-56728191/IL23A (−4505), PAN2 (−625); chr12:56842998-56843042/TIMELESS (+1
80); chr12:56882568-56882583/GLS2 (−395); chr12:57081831-57081863/PTGES3 (+231); chr12:57472235-574
72277/TMEM194A (+318); chr12:57608272-57608337/NXPH4 (−2273); chr12:57618740-57619085/SHMT2 (−44
43); chr12:57824835-57824865/INHBC (−3693); chr12:58004614-58004657/ARHGEF25 (+673); chr12:5801311
8-58013189/SLC26A10 (−539); chr12:58013355-58013420/SLC26A10 (−305); chr12:58013538-58013611/SLC2
6A10 (−118); chr12:58014571-58014820/SLC26A10 (+1003), B4GALNT1 (+12289); chr12:58021459-58021707/
B4GALNT1 (+5402), SLC26A10 (+7890); chr12:58025734-58025869/B4GALNT1 (+1183), SLC26A10 (+12109);
chr12:58026009-58026065/B4GALNT1 (+948); chr12:58158712-58158778/CYP27B1 (+723); chr12:62585013-
62585030/FAM19A2 (+1598); chr12:63543646-63543753/PPM1H (−215035), AVPR1A (+2890); chr12:6354440
2-63544498/PPM1H (−215785), AVPR1A (+2140); chr12:63544600-63544728/PPM1H (−215999), AVPR1A (+19
26); chr12:64784081-64784107/XPOT (−14059), SRGAP1 (+545553); chr12:65003768-65003812/RASSF3 (−50
3); chr12:65218559-65218727/TBC1D30 (+291); chr12:65219528-65219605/TBC1D30 (+1215), WIF1 (+29577
9); chr12:65564653-65564722/MSRB3 (−108079), LEMD3 (+1337); chr12:65672090-65672173/MSRB3 (−635);
chr12:66122996-66123284/HMGA2 (−95100), MSRB3 (+450373); chr12:66275569-66275657/HMGA2 (+57373),
LLPH (+248920); chr12:66563504-66563563/TMBIM4 (+273); chr12:67463646-67463696/GRIP1 (−390746), C
AND1 (−199390); chr12:70759865-70759910/KCNMB4 (−174); chr12:70760367-70760384/KCNMB4 (+314); chr
12:72666295-72666330/TRHDE (−216); chr12:72666907-72666997/TRHDE (+423); chr12:75601231-75601378/
KCNC2 (+2206); chr12:75601500-75601695/KCNC2 (+1913); chr12:76424205-76424269/KRR1 (−518819), PHL
DA1 (+1319); chr12:76425040-76425082/PHLDA1 (+495); chr12:77157930-77157949/ZDHHC17 (+86); chr12:7
9258879-79258965/SYT1 (−180511); chr12:81101798-81101900/MYF6 (+441); chr12:81102034-81102135/MY
F6 (+677); chr12:81102456-81102512/MYF5 (−8224), MYF6 (+1076); chr12:81107932-81107996/MYF5 (−2744);
chr12:81471615-81471753/ACSS3 (−125); chr12:82153255-82153283/PPFIA2 (−160); chr12:82752104-827521
61/CCDC59 (+66); chr12:83080941-83080990/TMTC2 (+32); chr12:85306282-85306322/SLC6A15 (+304); chr1
2:85306573-85306789/SLC6A15 (−75); chr12:85672287-85672326/ALX1 (−1729); chr12:93835438-93835512/U
BE2N (+551); chr12:93967216-93967274/CRADD (−103906), SOCS2 (+1764); chr12:95267194-95267247/TMC
C3 (−222897), NDUFA12 (+130290); chr12:95267681-95267706/TMCC3 (−223370), NDUFA12 (+129817); chr1
2:95466966-95467037/NR2C1 (+402); chr12:95941795-95942135/USP44 (+655); chr12:95942270-95942346/U
SP44 (+312); chr12:95942723-95942748/USP44 (−116); chr12:96252489-96252568/SNRPF (−180); chr12:9658
7990-96588010/ELK3 (−207); chr12:97379957-97379992/NEDD1 (+78974); chr12:98909268-98909291/TMPO
(−71); chr12:99139472-99139525/APAF1 (+100421); chr12:99288408-99288621/APAF1 (+249437); chr12:99288
937-99288961/APAF1 (+249871); chr12:100536769-100536816/ANKS1B (−158361), ACTR6 (−57781); chr12:10
1109992-101110039/ANO4 (−78358), GAS2L3 (+142527); chr12:101110315-101110358/ANO4 (−78037), GAS2
L3 (+142848); chr12:102036189-102036308/CHPT1 (−55168), MYBPC1 (+47540); chr12:103344940-10334497
9/PAH (−33579), ASCL1 (−6492); chr12:103350304-103350383/ASCL1 (−1108); chr12:103351986-103352051/A
SCL1 (+567); chr12:103352155-103352170/ASCL1 (+711); chr12:103352283-103352313/ASCL1 (+846); chr12:
103696160-103696193/STAB2 (−284892), ASCL1 (+344725); chr12:103888478-103888614/STAB2 (−92523), A
SCL1 (+537094); chr12:103889714-103889788/STAB2 (−91318), ASCL1 (+538299); chr12:104234273-1042343
31/NT5DC3 (+673); chr12:104458416-104458472/HCFC2 (+208); chr12:104609878-104610162/TXNRD1 (+46
1); chr12:104850537-104850577/CHST11 (−135); chr12:105351786-105351818/SLC41A2 (−29330), ALDH1L2
(+126539); chr12:106976780-106976842/RFX4 (−18104), POLR3B (+225375); chr12:106979590-106979636/RF
X4 (−15302), POLR3B (+228177); chr12:106980747-106980911/RFX4 (−14086), POLR3B (+229393); chr12:107
297125-107297169/C12orf23 (−52397), RIC8B (+128748); chr12:108079467-108079515/PWP1 (−99); chr12:108
155006-108155044/PRDM4 (−111); chr12:108169414-108169549/WSCD2 (−354029), ASCL4 (+1320); chr12:10
8237525-108237660/WSCD2 (−285918), ASCL4 (+69431); chr12:108238514-108238683/WSCD2 (−284912), A
SCL4 (+70437); chr12:108239130-108239244/WSCD2 (−284324), ASCL4 (+71025); chr12:108239603-1082396
45/WSCD2 (−283887), ASCL4 (+71462); chr12:108258131-108258145/WSCD2 (−265373), ASCL4 (+89976); ch
r12:108955282-108955347/ISCU (−979), SART3 (−150); chr12:109124246-109124419/CORO1C (+962); chr12:1
09125003-109125126/CORO1C (+230); chr12:109162494-109162538/CORO1C (−37221), SSH1 (+88843); chr
12:109491027-109491071/USP30 (+669); chr12:110150197-110150260/TRPV4 (+120983), MVK (+138729); ch
r12:110156231-110156484/TRPV4 (+114854), MVK (+144858); chr12:110841286-110841330/ANAPC7 (+227);
chr12:110906112-110906153/GPN3 (+393); chr12:110939835-110939888/RAD9B (−143), VPS29 (+54); chr12:
111471960-111472058/CUX2 (+181); chr12:111472196-111472356/CUX2 (+448); chr12:111472672-11147285
1/CUX2 (+934); chr12:111847214-111847305/SH2B3 (+3508), ATXN2 (+190220); chr12:111866818-11186689
2/SH2B3 (+23103), ATXN2 (+170625); chr12:112035535-112035577/ATXN2 (+1924), SH2B3 (+191804); chr12:
112450361-112450381/ERP29 (−781), TMEM116 (+652); chr12:112820214-112820247/C12orf51 (−335); chr12:
113013279-113013320/RPH3A (−216249), PTPN11 (+156764); chr12:113494416-113494430/DTX1 (−1239); ch
r12:113508119-113508199/DTX1 (+12497), RASAL1 (+65400); chr12:113900678-113900752/LHX5 (+9162), S
DSL (+40499); chr12:113901159-113901407/LHX5 (+8594), SDSL (+41067); chr12:113902018-113902041/LH
X5 (+7847), SDSL (+41814); chr12:113906875-113906998/LHX5 (+2940), SDSL (+46721); chr12:113909315-1
13909328/LHX5 (+555); chr12:113909503-113909534/LHX5 (+358); chr12:113909657-113909717/LHX5 (+190);
chr12:113913682-113913883/LHX5 (−3906); chr12:113914224-113914300/LHX5 (−4385); chr12:113917517-1
13917580/LHX5 (−7672), RBM19 (+486627); chr12:113917701-113917730/LHX5 (−7839), RBM19 (+486460); c
hr12:114075876-114075909/LHX5 (−166016), RBM19 (+328283); chr12:114404064-114404100/RBM19 (+94);
chr12:114834173-114834208/RBM19 (−430015), TBX5 (+9698); chr12:114838312-114838368/RBM19 (−43416
4), TBX5 (+5549); chr12:114843187-114843260/TBX5 (+665); chr12:114846947-114847042/TBX5 (−3106); chr
12:114847437-114847577/TBX5 (−3618); chr12:114878594-114878808/TBX5 (−34812), TBX3 (+243268); chr12:
114885184-114885371/TBX5 (−41389), TBX3 (+236691); chr12:114886431-114886560/TBX5 (−42607), TBX3
(+235473); chr12:115102508-115102687/TBX5 (−258709), TBX3 (+19371); chr12:115103155-115103249/TBX5
(−259313), TBX3 (+18767); chr12:115103711-115103980/TBX5 (−259957), TBX3 (+18123); chr12:115105189-1
15105395/TBX5 (−261403), TBX3 (+16677); chr12:115109568-115109885/TBX5 (−265838), TBX3 (+12242); chr
12:115123170-115123418/TBX3 (−1325); chr12:115124972-115125106/TBX3 (−3070); chr12:115129956-11513
0053/TBX3 (−8036); chr12:115138087-115138274/TBX3 (−16212); chr12:115173644-115173721/TBX3 (−51714);
chr12:115174204-115174372/TBX3 (−52319); chr12:116061886-116061975/TBX3 (−939962), MED13L (+6530
60); chr12:116715479-116715507/MED13L (−502); chr12:116946200-116946250/MED13L (−231234), MAP1LC
3B2 (−50961); chr12:116971186-116971210/MED13L (−256207), MAP1LC362 (−25988); chr12:117175676-1171
75712/RNFT2 (−402), C12orf49 (+149); chr12:117482952-117483055/TESC (+54247), FBXW8 (+134243); chr1
2:117771459-117771510/FBXO21 (−143185), NOS1 (+28122); chr12:117798455-117799069/NOS1 (+845); chr
12:117799279-117799330/NOS1 (+302); chr12:119212200-119212318/SRRM4 (−207041), SUDS3 (+397901);
chr12:119419324-119419368/SRRM4 (+46); chr12:120032431-120032454/PRKAB1 (−73318), TMEM233 (+117
9); chr12:120875971-120876009/COX6A1 (+86); chr12:120972385-120972414/RNF10 (+268); chr12:12101874
5-121018809/POP5 (+424); chr12:121078324-121078357/CABP1 (−81); chr12:121079341-121079420/CABP1
(+959); chr12:121341588-121341649/SPPL3 (+536); chr12:121530597-121530651/OASL (−53844), P2RX7 (−39
998); chr12:121735577-121735630/CAMKK2 (+507); chr12:121837619-121837660/RNF34 (−262); chr12:12201
6655-122016849/KDM2B (+2168), RNF34 (+178850); chr12:122065765-122065827/TMEM120B (−84862), ORA
I1 (+1341); chr12:122227929-122227995/RHOF (+3632), TMEM120B (+77304); chr12:122231057-122231148/
RHOF (+491); chr12:122240919-122240973/SETD18 (−1692); chr12:122442261-122442329/BCL7A (−17566),
WDR66 (+85832); chr12:122669142-122669185/B3GNT4 (−19064), IL31 (−10418); chr12:122906885-12290690
4/CLIP1 (+221); chr12:123259687-123259752/CCDC62 (+664); chr12:123450922-123450965/ABCB9 (+112); c
hr12:123518668-123518818/ARL6IP4 (+53863), PITPNM2 (+76292); chr12:123717466-123717661/C12orf65
(−280); chr12:123873289-123873313/SETD8 (+4597), RILPL2 (+47963); chr12:123915938-123916040/RILPL2
(+5275), SETD8 (+47285); chr12:124068959-124068990/TMED2 (−101); chr12:124155353-124155457/TCTN2 (−2
55); chr12:124246390-124246445/DNAH10 (−624); chr12:124457060-124457082/ZNF664 (−691); chr12:124781
163-124781200/NCOR2 (+270828), ZNF664 (+323420); chr12:124873546-124873721/NCOR2 (+178376), ZNF
664 (+415872); chr12:124874062-124874106/NCOR2 (+177926), ZNF664 (+416322); chr12:124876991-12487
7052/NCOR2 (+174988), ZNF664 (+419260); chr12:124941845-124941950/NCOR2 (+110112), ZNF664 (+484
136); chr12:124942236-124942316/NCOR2 (+109734), ZNF664 (+484514); chr12:125005721-125005844/NCO
R2 (+46227), ZNF664 (+548021); chr12:125039318-125039419/NCOR2 (+12641), ZNF664 (+581607); chr12:1
25052213-125052265/NCOR2 (−229); chr12:125353642-125353738/SCARB1 (−5171), UBC (+45897); chr12:12
5670261-125670334/TMEM132B (−140864), AACS (+120373); chr12:127210778-127210895/NONE; chr12:127
211033-127211063/NONE; chr12:127630654-127630798/NONE; chr12:127940393-127940454/TMEM132C (−8
11524); chr12:127940558-127940587/TMEM132C (−811375); chr12:127940688-127940852/TMEM132C (−8111
78); chr12:128675290-128675354/TMEM132C (−76626); chr12:128873180-128873290/TMEM132C (+121287),
SLC15A4 (+435306); chr12:129338109-129338192/GLT1D1 (+70); chr12:129338826-129338851/GLT1D1 (+75
8); chr12:130387550-130387796/TMEM132D (+539); chr12:130388018-130388135/TMEM132D (+135); chr12:
130388657-130388944/TMEM132D (−589); chr12:130526888-130526931/TMEM132D (−138698), FZD10 (−1200
94); chr12:130529157-130529217/TMEM132D (−140975), FZD10 (−117817); chr12:130646654-130646755/FZD
10 (−299); chr12:130647133-130647214/FZD10 (+170); chr12:130647435-130647543/FZD10 (+485); chr12:130
647909-130647950/FZD10 (+926); chr12:130648125-130648818/PIWIL1 (−173961), FZD10 (+1468); chr12:130
649126-130649260/PIWIL1 (−173240), FZD10 (+2189); chr12:130765916-130765932/PIWIL1 (−56509), FZD10
(+118920); chr12:130823873-130823917/PIWIL1 (+1462), RIMBP2 (+178567); chr12:131260513-131260561/RI
MBP2 (−258075), STX2 (+63274); chr12:131464567-131464617/SFSWAP (−731043), GPR133 (+26140); chr12:
131783246-131783291/SFSWAP (−412366), GPR133 (+344817); chr12:131783422-131783702/SFSWAP (−412
073), GPR133 (+345110); chr12:132146199-132146540/SFSWAP (−49265), GPR133 (+707918); chr12:132146
653-132146752/SFSWAP (−48932), GPR133 (+708251); chr12:132312830-132312856/MMP17 (−98); chr12:132
337633-132337686/ULK1 (−41619), MMP17 (+24719); chr12:132381558-132381695/PUS1 (−32186), ULK1 (+2
348); chr12:132628365-132628401/NOC4L (−610), DDX51 (+497); chr12:132657818-132657946/NOC4L (+288
89), GALNT9 (+248023); chr12:132905535-132905551/GALNT9 (+362); chr12:132905652-132905713/GALNT9
(+222); chr12:132906025-132906202/GALNT9 (−209); chr12:132906354-132906392/GALNT9 (−468); chr12:13
2970577-132970683/P2RX2 (−224773), GALNT9 (−64725); chr12:133016005-133016128/P2RX2 (−179336), GA
LNT9 (−110162); chr12:133021467-133021524/P2RX2 (−173907), GALNT9 (−115591); chr12:133053668-13305
3764/GALNT9 (−147811), P2RX2 (−141687); chr12:133175547-133175588/GALNT9 (−269663), P2RX2 (−19835);
chr12:133195229-133195428/P2RX2 (−74); chr12:133195940-133195965/P2RX2 (+550); chr12:133345275-13
3345321/ANKLE2 (−6847), GOLGA3 (+59990); chr12:133481383-133481489/CHFR (−17232), ZNF605 (+51456);
chr12:133484965-133485046/CHFR (−20802), ZNF605 (+47886); chr12:133485348-133485439/CHFR (−2119
0), ZNF605 (+47498); chr12:133485690-133485815/CHFR (−21549), ZNF605 (+47139); chr12:133553643-1335
53700/ZNF605 (−20780), ZNF26 (−9384); chr12:133613775-133613813/ZNF84 (−186); chr12:133707261-13370
7315/ZNF10 (+74); chr13:19918724-19918765/TUBA3C (−162753), TPTE2 (+192158); chr13:19919069-199191
25/TUBA3C (−163105), TPTE2 (+191806); chr13:20176683-20176804/TPTE2 (−65841), MPHOSPH8 (−31044);
chr13:20438932-20439012/ZMYM5 (−1196); chr13:20532107-20532173/ZMYM2 (−670); chr13:20702495-20702
531/GJA3 (+32670), ZMYM2 (+169703); chr13:20716423-20716640/GJA3 (+18651), ZMYM2 (+183722); chr13:
20763427-20763462/GJA3 (−28262), GJB2 (+3669); chr13:20767761-20767827/GJB2 (−680); chr13:20805588-
20805728/GJB6 (+876); chr13:20875629-20876019/GJB6 (−69290), CRYL1 (+224188); chr13:21295855-21295
877/IL17D (+18384), N6AMT2 (+52191); chr13:21520358-21520551/XPO4 (−43542), LATS2 (+115267); chr13:2
1635756-21635777/LATS2 (−45); chr13:21750979-21751009/SKA3 (−253), MRP63 (+622); chr13:21872852-218
72941/SKA3 (−122156), ZDHHC20 (+160611); chr13:22033216-22033256/ZDHHC20 (+272); chr13:22177982-2
2178023/EFHA1 (+352); chr13:23500637-23500876/SGCG (−254303); chr13:23734298-23734814/SGCG (−205
04); chr13:24463000-24463056/C1QTNF9B-AS1 (−564), MIPEP (+559); chr13:24463281-24463332/C1QTNF9B-
AS1 (−285), MIPEP (+280); chr13:24478017-24478110/SPATA13 (−256797), C1QTNF9B (−6919); chr13:24553
864-24553924/SPATA13 (−180967), C1QTNF9B (−82749); chr13:24839234-24839388/C1QTNF9 (−44405), SPA
TA13 (+104450); chr13:25086908-25086946/PARP4 (+21); chr13:25115648-25115693/ATP12A (−138878), PA
RP4 (−28723); chr13:25115907-25116223/ATP12A (−138484), PARP4 (−29117); chr13:25254841-25254861/AT
P12A (+302); chr13:25319905-25320108/RNF17 (−18294), ATP12A (+65458); chr13:25320246-25320375/RNF
17 (−17990), ATP12A (+65762); chr13:25320540-25320613/RNF17 (−17724), ATP12A (+66028); chr13:2532102
9-25321112/RNF17 (−17230), ATP12A (+66522); chr13:25322194-25322238/RNF17 (−16085), ATP12A (+6766
7); chr13:25506123-25506195/PABPC3 (−164117), CENPJ (−9074); chr13:25592566-25592593/CENPJ (−95495),
PABPC3 (−77696); chr13:25593040-25593061/CENPJ (−95966), PABPC3 (−77225); chr13:25621206-2562126
3/CENPJ (−124150), PABPC3 (−49041); chr13:25744735-25745300/PABPC3 (+74742), MTMR6 (+116686); chr
13:25745595-25745726/PABPC3 (+75385), MTMR6 (+116043); chr13:25746081-25746119/PABPC3 (+75824),
MTMR6 (+115604); chr13:25794284-25794425/MTMR6 (+67349), PABPC3 (+124079); chr13:25875842-25875
892/NUPL1 (+201); chr13:25876011-25876046/NUPL1 (+363); chr13:25946018-25946300/ATP8A2 (−50); chr13:
25946674-25946801/ATP8A2 (+529); chr13:26042708-26042768/ATP8A2 (+96529), SHISA2 (+582460); chr13:
26043171-26043340/ATP8A2 (+97047), SHISA2 (+581942); chr13:26551514-26551585/SHISA2 (+73648), AT
P8A2 (+605341); chr13:26586752-26586839/SHISA2 (+38402), ATP8A2 (+640587); chr13:26624388-2662442
5/SHISA2 (+791); chr13:26625198-26625266/SHISA2 (−34); chr13:26625716-26625841/SHISA2 (−581); chr13:2
6647639-26647724/SHISA2 (−22484), RNF6 (+148826); chr13:26760763-26760795/SHISA2 (−135581), RNF6
(+35729); chr13:27131829-27131859/WASF3 (+4); chr13:27132310-27132629/WASF3 (+630); chr13:27825351-
27825385/RPL21 (−324); chr13:28194183-28194268/POLR1D (−1690), LNX2 (+494); chr13:28364095-2836415
0/GSX1 (−2657); chr13:28364696-28364723/GSX1 (−2070); chr13:28366602-28366664/GSX1 (−147); chr13:283
67039-28367253/GSX1 (+366); chr13:28367946-28368153/PDX1 (−126118), GSX1 (+1270); chr13:28369071-2
8369161/PDX1 (−125052), GSX1 (+2336); chr13:28370424-28370502/PDX1 (−123705), GSX1 (+3683); chr13:2
8493310-28493345/PDX1 (−840); chr13:28498358-28498986/PDX1 (+4504), CDX2 (+44645); chr13:28500895-
28500954/PDX1 (+6757), CDX2 (+42392); chr13:28501125-28501150/PDX1 (+6970), CDX2 (+42179); chr13:2
8501937-28502077/PDX1 (+7839), CDX2 (+41310); chr13:28502742-28502812/PDX1 (+8609), CDX2 (+40540);
chr13:28503274-28503358/PDX1 (+9148), CDX2 (+40001); chr13:28535270-28535305/CDX2 (+8029), PDX1
(+41120); chr13:28541100-28541179/CDX2 (+2177), PDX1 (+46972); chr13:28544585-28544665/CDX2 (−1308);
chr13:28549945-28550239/CDX2 (−6775), PRHOXNB (+12682); chr13:28551549-28551658/CDX2 (−8287), P
RHOXNB (+11170); chr13:28552385-28552659/CDX2 (−9205), PRHOXNB (+10252); chr13:28554359-2855454
3/CDX2 (−11134), PRHOXNB (+8323); chr13:28674227-28674397/FLT3 (+417); chr13:29068986-29068993/FL
T1 (+275); chr13:29106815-29106898/POMP (−126284), FLT1 (−37592); chr13:29107064-29107233/POMP (−12
5992), FLT1 (−37884); chr13:30688968-30689006/UBL3 (−264167), KATNAL1 (+192176); chr13:30707204-3070
7244/UBL3 (−282404), KATNAL1 (+173939); chr13:31019984-31020055/KATNAL1 (−138857), HMGB1 (+20061);
chr13:31248065-31248116/ALOX5AP (−39524), USPL1 (+56261); chr13:31480504-31480656/ALOX5AP (+19
2965), HSPH1 (+255537); chr13:31480870-31481052/ALOX5AP (+193346), HSPH1 (+255156); chr13:3260532
5-32605442/FRY (−53); chr13:32605624-32605674/FRY (+212); chr13:33161272-33161316/PDS5B (+730); chr
13:33590658-33591066/KL (+291); chr13:34116683-34116717/RFC3 (−275506), STARD13 (−256799); chr13:35
324657-35324665/NBEA (−191763), RFC3 (+932455); chr13:35517004-35517052/NBEA (+604); chr13:355173
73-35517409/NBEA (+967); chr13:36045213-36045321/MAB21L1 (+470115), NBEA (+528843); chr13:3604933
4-36049572/MAB21L1 (+465929), NBEA (+533029); chr13:36049759-36049787/MAB21L1 (+465609), NBEA
(+533349); chr13:36050774-36050905/MAB21L1 (+464542), NBEA (+534416); chr13:36052553-36052827/MAB2
1L1 (+462692), NBEA (+536266); chr13:36705566-36705621/DCLK1 (−80); chr13:36728991-36729246/DCLK1
(−23605), CCDC169-SOHLH2 (+142873); chr13:36871607-36871670/CCDC169-SOHLH2 (+353); chr13:368719
72-36872032/CCDC169-SOHLH2 (−10); chr13:36920224-36920266/SPG20 (+401); chr13:36920387-36920464/
SPG20 (+220); chr13:37005581-37005720/CCNA1 (−758); chr13:37006202-37006433/CCNA1 (−91); chr13:370
06658-37006733/CCNA1 (+287); chr13:37248149-37248243/SERTM1 (+147); chr13:37248994-37249039/SER
TM1 (+968); chr13:37494107-37494124/SMAD9 (+293); chr13:38443432-38443479/TRPC4 (+483); chr13:3844
3716-38443934/TRPC4 (+114); chr13:38444151-38444226/TRPC4 (−250); chr13:39261783-39261928/FREM2
(+683); chr13:39262030-39262129/FREM2 (+907); chr13:43148375-43148495/TNFSF11 (+144); chr13:4359763
5-43597689/DNAJC15 (+300); chr13:44359980-44360043/SERP2 (−587966), ENOX1 (−156399); chr13:443602
79-44360467/SERP2 (−587605), ENOX1 (−156760); chr13:44360794-44360995/SERP2 (−587083), ENOX1 (−15
7282); chr13:44361593-4436183/SERP2 (−586340), ENOX1 (−158025); chr13:44947700-44947725/SERP2 (−2
65); chr13:45150738-45150766/TSC22D1 (−51); chr13:45492239-45492271/TSC22D1 (−341554), NUFIP1 (+71
358); chr13:46039471-46039531/COG3 (+471); chr13:46189934-46190006/SPERT (−86476), COG3 (+150940);
chr13:46425555-46425575/SIAH3 (+281); chr13:46425789-46425855/SIAH3 (+24); chr13:46626241-46626295/
ZC3H13 (+626); chr13:47012298-47012316/KIAA0226L (−50672), ESD (+359060); chr13:49107168-49107192/
RCBTB2 (+136); chr13:49550736-49550781/FNDC3A (+711); chr13:49794926-49795540/MLNR (+759); chr13:
49822278-49822314/CDADC1 (+249); chr13:50070153-50070315/PHF11 (+433); chr13:50070658-50070684/P
HF11 (+870); chr13:50159828-50159866/RCBTB1 (−128); chr13:50202847-50202964/ARL11 (+471); chr13:506
54261-50654311/ST13P4 (−91868), KCNRG (+64896); chr13:50701689-50701959/ST13P4 (−44330), KCNRG
(+112434); chr13:50706908-50707042/ST13P4 (−39179), KCNRG (+117585); chr13:50707621-50707711/ST13P
4 (−38488), KCNRG (+118276); chr13:51417603-51417906/RNASEH2B (−66059), ST13P4 (+671601); chr13:52
025886-52025906/INTS6 (+1379), SERPINE3 (+110728); chr13:52027224-52027258/INTS6 (+34); chr13:5276
8301-52768350/NEK3 (−34330), THSD1 (+212303); chr13:52980515-52980573/THSD1 (+85); chr13:53174624-
53174656/HNRNPA1L2 (−16965), CKAP2 (+145145); chr13:53191405-53191417/HNRNPA1L2 (−194); chr13:53
313314-53313512/LECT1 (+534); chr13:53313613-53313677/LECT1 (+302); chr13:53313950-53314030/LECT
1 (−43); chr13:53420029-53420065/LECT1 (−106100), PCDH8 (+2728); chr13:53420208-53420877/LECT1 (−10
6596), PCDH8 (+2232); chr13:53421000-53421826/LECT1 (−107466), PCDH8 (+1362); chr13:53422252-53422
432/PCDH8 (+433); chr13:53774952-53775398/OLFM4 (+172299); chr13:53776135-53776207/OLFM4 (+1732
95); chr13:58205000-58205044/PCDH17 (−767); chr13:58206232-58206452/PCDH17 (+553); chr13:58206772-
58206861/PCDH17 (+1028); chr13:58207401-58207567/PCDH17 (+1695); chr13:58207814-58207891/PCDH1
7 (+2064); chr13:61165578-61165696/TDRD3 (+194210), PCDH20 (+824018); chr13:61987962-61987997/PCD
H20 (+1675); chr13:61989193-61989219/PCDH20 (+449); chr13:67804531-67804644/PCDH9 (−120); chr13:67
804768-67804863/PCDH9 (−348); chr13:67805286-67805379/PCDH9 (−865); chr13:70681778-70681866/KLHL
1 (+803); chr13:73632865-73632890/KLF5 (−264); chr13:76054939-76055007/TBC1D4 (+1277); chr13:7760137
5-77601407/FBXL3 (−60); chr13:79161838-79161907/EDNRB (−667970), POU4F1 (+15822); chr13:79169716-7
9169849/EDNRB (−675880), POU4F1 (+7912); chr13:79170114-79170211/EDNRB (−676260), POU4F1 (+7532);
chr13:79170384-79170467/EDNRB (−676523), POU4F1 (+7269); chr13:79175944-79176077/EDNRB (−68210
8), POU4F1 (+1684); chr13:79176431-79176566/EDNRB (−682596), POU4F1 (+1196); chr13:79177185-791773
05/POU4F1 (+450); chr13:79177623-79177650/POU4F1 (+58); chr13:79177762-79177885/POU4F1 (−129); chr
13:79182115-79182150/POU4F1 (−4438); chr13:84453842-84454005/SLITRK1 (+2604); chr13:84454250-8445
4310/SLITRK1 (+2248); chr13:88323831-88323867/SLITRK5 (−1021); chr13:88324519-88324575/SLITRK5 (−32
3); chr13:88324747-88324763/STLIRK5 (−115); chr13:88326708-88326936/SLITRK5 (+1952); chr13:91827441-
91827599/GPC5 (−223415); chr13:92051514-92051772/GPC5 (+708); chr13:93879776-93879918/GPC6 (+769);
chr13:93880217-93880533/GPC6 (+1297); chr13:93880738-93880793/GPC6 (+1688); chr13:95201950-95202
013/DCT (−70046), TGDS (+46529); chr13:95248482-95248546/TGDS (−3); chr13:95354840-95355116/SOX21
(+9411), GPR180 (+100874); chr13:95363452-95363493/SOX21 (+916); chr13:95363960-95364064/SOX21
(+377); chr13:95364291-95364329/SOX21 (+79); chr13:95365457-95365497/SOX21 (−1088); chr13:95620782-95
620853/SOX21 (−256429), ABCC4 (+332869); chr13:96056516-96056547/CLDN10 (−148415), ABCC4 (−102845);
chr13:96130850-96130913/ABCC4 (−177195), CLDN10 (−74065); chr13:96204668-96204922/CLDN10 (−152);
chr13:96294364-96294542/DZIP1 (+2504), CLDN10 (+89506); chr13:96296346-96296372/DZIP1 (+598); chr1
3:96296556-96296840/DZIP1 (+259); chr13:96296950-96296991/DZIP1 (−14); chr13:99405000-99405089/SLC
15A1 (−116); chr13:99853278-99853318/UBAC2 (+619); chr13:100259159-100259185/CLYBL (+253); chr13:10
0310843-100310935/CLYBL (+51970), ZIC5 (+313289); chr13:100547894-100548075/ZIC5 (+76193), CLYBL
(+289066); chr13:100548305-100548324/ZIC5 (+75863), CLYBL (+289396); chr13:100548512-100548649/ZIC5
(+75597), CLYBL (+289662); chr13:100608036-100608448/ZIC5 (+15936), CLYBL (+349323); chr13:10060880
5-100608838/ZIC5 (+15356), CLYBL (+349903); chr13:100620485-100620515/ZIC5 (+3678), CLYBL (+361581);
chr13:100620620-100620631/ZIC5 (+3552), CLYBL (+361707); chr13:100622031-100622155/ZIC5 (+2085), C
LYBL (+363174); chr13:100624233-100624323/ZIC5 (−100); chr13:100627928-100628071/ZIC5 (−3822); chr13:
100630299-100630328/ZIC2 (−3712); chr13:100634745-100634940/ZIC2 (+817); chr13:100635330-100635398/
PCCA (−105905), ZIC2 (+1338); chr13:100635518-100635597/PCCA (−105711), ZIC2 (+1532); chr13:10063611
1-100636130/PCCA (−105148), ZIC2 (+2095); chr13:100641638-100641769/PCCA (−99565), ZIC2 (+7678); chr
13:100642486-100642614/PCCA (−98719), ZIC2 (+8524); chr13:100643667-100643790/PCCA (−97540), ZIC2
(+9703); chr13:100644180-100644218/PCCA (−97070), ZIC2 (+10173); chr13:100649583-100649672/PCCA (−9
1641), ZIC2 (+15602); chr13:100649885-100649944/PCCA (−91354), ZIC2 (+15889); chr13:100926036-100926
147/PCCA (+184823), A2LD1 (+259964); chr13:101327146-101327189/TMTC4 (−65); chr13:102068358-10206
8417/NALCN (+425); chr13:102068646-102068811/NALCN (+84); chr13:102069071-102069256/NALCN (−351);
chr13:102568573-102568659/ITGBL1 (+463650), FGF14 (+485508); chr13:102568768-102569203/ITGBL1 (+4
64020), FGF14 (+485138); chr13:103426388-103426431/METTL21C (−79556), KDELC1 (+24994); chr13:10345
2645-103452670/KDELC1 (−1254); chr13:108518354-108518444/FAM155A (+1061); chr13:108519431-108519
564/FAM155A (−38); chr13:108519746-108519901/FAM155A (−364); chr13:108520097-108520309/FAM155A
(−743); chr13:108520535-108520701/FAM155A (−1158); chr13:108520826-108520915/FAM155A (−1411); chr13:
108867835-108867873/ABHD13 (−2909), LIG4 (+28); chr13:109147866-109147999/MYO16 (−100567), TNFSF1
3B (+225956); chr13:109148279-109148376/MYO16 (−100172), TNFSF13B (+226351); chr13:109792906-1097
93284/MYO16 (+544595), IRS2 (+645819); chr13:109793427-109793463/MYO16 (+544945), IRS2 (+645469);
chr13:110052679-110052721/IRS2 (+386214), MYO16 (+804200); chr13:110437505-110437578/IRS2 (+1372);
chr13:110438246-110438287/IRS2 (+647); chr13:110950784-110950839/IRS2 (−511898), COL4A1 (+8684); ch
r13:110959054-110959517/COL4A2 (−345), COL4A1 (+210); chr13:110959649-110959752/COL4A1 (−205), CO
L4A2 (+70); chr13:110960143-110960344/COL4A1 (−748), COL4A2 (+613); chr13:110964145-110964286/COL
4A1 (−4720), COL4A2 (+4585); chr13:111214430-111214475/RAB20 (−382); chr13:111365679-111365806/ING
1 (−1616); chr13:111368008-111368030/ING1 (+660); chr13:111464780-111464916/ARHGEF7 (−302776), ING
1 (+97489); chr13:111465044-111465089/ARHGEF7 (−302557), ING1 (+97708); chr13:111718992-111719070/
ARHGEF7 (−48593), ING1 (+351672); chr13:111768118-111768416/ARHGEF7 (+643); chr13:111768541-1117
68604/ARHGEF7 (+949); chr13:112547631-112547690/SOX1 (−174252), C13orf16 (+574646); chr13:11254801
2-112548051/SOX1 (−173881), C13orf16 (+575017); chr13:112548550-112548642/SOX1 (−173317), C13orf16
(+575581); chr13:112708005-112708307/SOX1 (−13757), C13orf16 (+735141); chr13:112710253-112710359/S
OX1 (−11607), C13orf16 (+737291); chr13:112710476-112710635/SOX1 (−11357), C13orf16 (+737541); chr13:1
12711294-112711375/SOX1 (−10578), C13orf16 (+738320); chr13:112715719-112715803/SOX1 (−6152), C13o
rf16 (+742746); chr13:112717243-112717322/SOX1 (−4630); chr13:112721027-112721136/SOX1 (−831); chr13:
112721475-112722094/SOX1 (−128); chr13:112722221-112722274/SOX1 (+335); chr13:112722403-11272259
7/SOX1 (+587); chr13:112723070-112723154/SPACA7 (−307539), SOX1 (+1199); chr13:112726293-11272643
5/SPACA7 (−304287), SOX1 (+4451); chr13:112726729-112726796/SPACA7 (−303888), SOX1 (+4850); chr13:
112727220-112727698/SPACA7 (−303192), SOX1 (+5546); chr13:112727879-112727961/SPACA7 (−302731),
SOX1 (+6007); chr13:112728201-112728335/SPACA7 (−302383), SOX1 (+6355); chr13:112732766-112732827/
SPACA7 (−297854), SOX1 (+10884); chr13:112758373-112758495/SPACA7 (−272217), SOX1 (+36521); chr13:
112758784-112758867/SPACA7 (−271825), SOX1 (+36913); chr13:112759034-112759111/SPACA7 (−271578),
SOX1 (+37160); chr13:112759781-112760006/SPACA7 (−270757), SOX1 (+37981); chr13:112985423-112985
452/SPACA7 (−45213), SOX1 (+263525); chr13:112985553-112985585/SPACA7 (−45082), SOX1 (+263656); ch
r13:113528543-113528575/MCF2L (−94976), ATP11A (+183916); chr13:113652295-113652318/F7 (−107798),
MCF2L (+28772); chr13:113734977-113735033/F7 (−25100), MCF2L (+111470); chr13:113763950-113764088/
F10 (−13094), F7 (+3914); chr13:113764355-113764766/F10 (−12552), F7 (+4456); chr13:113765134-11376538
7/F10 (−11852), F7 (+5156); chr13:113807392-113807542/PROZ (−5501), F10 (+30354); chr13:113951118-113
951172/LAMP1 (−324); chr13:113951396-113951413/LAMP1 (−64); chr13:113951716-113951766/LAMP1 (+272);
chr13:114017945-114017991/GRTP1 (+495); chr13:114145249-114145270/TMCO3 (−48); chr13:114146137-
114146160/TMCO3 (+841); chr13:114239224-114239246/TFDP1 (+179); chr13:114261985-114262074/TFDP1
(+22974), ATP4B (+50483); chr13:114462807-114462866/FAM70B (+621); chr13:114566271-114566409/GAS
6 (+706); chr13:114830746-114830818/GAS6 (−263736), RASA3 (+67313); chr13:114875934-114876221/GAS
6 (−309032), RASA3 (+22017); chr13:114894153-114894280/GAS6 (−327171), RASA3 (+3878); chr13:1148960
49-114896191/GAS6 (−329074), RASA3 (+1975); chr13:114932050-114932215/CDC16 (−68229), RASA3 (−340
38); chr13:115046956-115047005/UPF3A (−78); chr14:19403296-19403330/OR11H12 (+25719), OR11H2 (+77
9178); chr14:20929224-20929256/TMEM55B (+397); chr14:20929539-20929605/TMEM55B (+65); chr14:21091
112-21091156/RNASE12 (−32152), OR6S1 (+18716); chr14:21092167-21092216/RNASE12 (−33210), OR6S1
(+17658); chr14:21094032-21094124/RNASE12 (−35096), OR6S1 (+15772); chr14:21101011-21101037/RNASE
12 (−42042), OR6S1 (+8826); chr14:21121217-21121262/RNASE4 (−35692), OR6S1 (−11390); chr14:21121917-
21121970/RNASE4 (−34988), OR6S1 (−12094); chr14:21493197-21493230/NDRG2 (+50); chr14:21494070-214
94160/TPPP2 (−4230), NDRG2 (−851); chr14:21539969-21540023/C14orf176 (−27100), ARHGEF40 (+1469); ch
r14:21852134-21852194/SUPT16H (+261); chr14:21924263-21924333/CHD8 (−24431), RAB2B (+20834); chr1
4:21945659-21945719/RAB2B (−557), TOX4 (+354); chr14:21994132-21994155/METTL3 (−14687), SALL2 (+11
193); chr14:23235774-23235832/OXA1L (+72); chr14:23299038-23299065/MRPL52 (−40); chr14:23305834-233
05961/MMP14 (+105); chr14:23398436-23398497/PRMT5 (+194); chr14:23770640-23770682/HOMEZ (−15352),
BCL2L2-PABPN1 (−5310); chr14:23821281-23821317/SLC22A17 (+361); chr14:23821576-23821601/SLC22A
17 (+71); chr14:23834788-23835314/EFS (−209); chr14:24020690-24020704/THTPA (−4501), ZFHX2 (+161); ch
r14:24025151-24025214/ZFHX2 (−4325), THTPA (−15); chr14:24045278-24045385/AP1G2 (−8053), JPH4 (+267
7); chr14:24045565-24045917/AP1G2 (−8462), JPH4 (+2268); chr14:24046361-24046394/AP1G2 (−9099), JPH
4 (+1631); chr14:24521457-24521486/CPNE6 (−19264), DHRS4 (+98507); chr14:24563712-24563760/PCK2
(+253); chr14:24601570-24601641/PSME1 (−3772), FITM1 (+931); chr14:24605275-24605296/PSME1 (−92); chr1
4:24640785-24641009/REC8 (−337); chr14:24641361-24641413/REC8 (+153); chr14:24711570-24711617/TIN
F2 (+286); chr14:24780761-24780824/CIDEB (−217), LTB4R (+88); chr14:24801500-24801528/ADCY4 (+2763),
LTB4R (+20809); chr14:24802022-24802151/ADCY4 (+2190), LTB4R (+21382); chr14:24804351-24804424/A
DCY4 (−111); chr14:24912068-24912085/SDR39U1 (−70); chr14:26674084-26674208/NOVA1 (+392814); chr14:
27066052-27066140/NOVA1 (+864); chr14:28733652-28733668/FOXG1 (−502627); chr14:29231186-2923128
3/FOXG1 (−5052); chr14:29235309-29235341/FOXG1 (−962); chr14:29235752-29235826/FOXG1 (−498); chr14:
29236823-29236859/FOXG1 (+554); chr14:29237067-29237139/FOXG1 (+816); chr14:29237267-29237282/F
OXG1 (+988); chr14:29237444-29237573/FOXG1 (+1222); chr14:29243670-29243730/FOXG1 (+7413); chr14:
29254440-29254611/FOXG1 (+18239); chr14:31495850-31495898/STRN3 (−267); chr14:32547388-32547438/
ARHGAP5 (+918); chr14:32670957-32670996/AKAP6 (−127502), ARHGAP5 (+124482); chr14:33402225-3340
2367/NPAS3 (−6163), AKAP6 (+603817); chr14:34269792-34269840/EGLN3 (+150468), NPAS3 (+861357); chr
14:35591674-35591704/PPP2R3C (−170), KIAA0391 (−87); chr14:35873764-35873798/NFKBIA (+179); chr14:3
6003957-36004080/INSM2 (+771); chr14:36004735-36004821/INSM2 (+1530), RALGAPA1 (+273654); chr14:3
6004922-36005011/INSM2 (+1719), RALGAPA1 (+273465); chr14:36789347-36789412/MBIP (+502); chr14:36
974928-36974967/MBIP (−185066), NKX2-1 (+14482); chr14:36975229-36975280/MBIP (−185373), NKX2-1 (+1
4175); chr14:36975640-36975682/MBIP (−185779), NKX2-1 (+13769); chr14:36977930-36977974/MBIP (−1880
70), NKX2-1 (+11478); chr14:36982696-36982869/MBIP (−192901), NKX2-1 (+6647); chr14:36983600-3698367
3/MBIP (−193755), NKX2-1 (+5793); chr14:36986472-36987198/MBIP (−196953), NKX2-1 (+2595); chr14:36989
341-36989403/NKX2-1 (+58); chr14:36991033-36991094/NKX2-1 (−1634); chr14:36991778-36991988/NKX2-1
(−2453); chr14:36992164-36992221/NKX2-1 (−2763); chr14:36993488-36993693/NKX2-1 (−4161); chr14:370491
27-37049158/NKX2-1 (−59713), NKX2-8 (+2643); chr14:37050040-37050094/NKX2-1 (−60637), NKX2-8 (+1719);
chr14:37053134-37053322/NKX2-8 (−1442); chr14:37053498-37053600/NKX2-8 (−1763); chr14:37116295-371
16552/NKX2-8 (−64638), PAX9 (−10349); chr14:37116666-37116771/NKX2-8 (−64933), PAX9 (−10054); chr14:3
7124194-37124481/PAX9 (−2435); chr14:37125652-37125763/PAX9 (−1065); chr14:37126714-37126964/PAX9
(+66); chr14:37127780-37128012/PAX9 (+1123), SLC25A21 (+513969); chr14:37132062-37132290/PAX9 (+54
03), SLC25A21 (+509689); chr14:37132554-37132602/PAX9 (+5805), SLC25A21 (+509287); chr14:37135636-
37135739/PAX9 (+8915), SLC25A21 (+506177); chr14:37136121-37136293/PAX9 (+9434), SLC25A21 (+5056
58); chr14:37145583-37145647/PAX9 (+18842), SLC25A21 (+496250); chr14:37666686-37666726/SLC25A21
(−24841), FOXA1 (+397783); chr14:38057418-38057438/SLC25A21 (−415563), FOXA1 (+7061); chr14:3806091
7-38061159/SLC25A21 (−419173), FOXA1 (+3451); chr14:38061290-38061332/SLC25A21 (−419446), FOXA1
(+3178); chr14:38066924-38066969/FOXA1 (−2458); chr14:38067530-38067610/FOXA1 (−3081); chr14:3806788
0-38067934/FOXA1 (−3418); chr14:38071297-38071341/TTC6 (−193156), FOXA1 (−6830); chr14:38080313-380
80668/TTC6 (−183984), FOXA1 (−16002); chr14:38091479-38091550/TTC6 (−172960), FOXA1 (−27026); chr14:
38680046-38680115/SSTR1 (+2877), CLEC14A (+45493); chr14:38680721-38680742/SSTR1 (+3528), CLEC1
4A (+44842); chr14:38724526-38724545/CLEC14A (+1038), SSTR1 (+47332); chr14:38725360-38725454/CLE
C14A (+167); chr14:42074587-42074668/LRFN5 (−2136); chr14:42075323-42075396/LRFN5 (−1404); chr14:42
076044-42076105/LRFN5 (−689); chr14:42077368-42077488/LRFN5 (+664); chr14:42078765-42078799/LRFN
5 (+2018); chr14:45381560-45381569/FSCB (−405066), FAM179B (−49851); chr14:45431149-45431187/FAM17
9B (−248); chr14:45431696-45431722/FAM179B (+293); chr14:48143537-48143617/MDGA2 (+580); chr14:481
43719-48143797/MDGA2 (+399); chr14:48143958-48144358/MDGA2 (−1); chr14:48144764-48145236/MDGA2
(−843); chr14:48145362-48145469/MDGA2 (−1259); chr14:50154784-50154840/POLE2 (+286); chr14:50160193-
50160247/KLHDC1 (+397); chr14:50582908-50582970/METTL21D (+358); chr14:50698742-50698767/SOS2
(−656); chr14:51134674-51134749/SAV1 (+311); chr14:51296786-51296865/SAV1 (−161803), NIN (+1013); chr1
4:51338732-51339047/ABHD12B (+12); chr14:51560714-51560770/PYGL (−149494), TRIM9 (+1680); chr14:52
535027-52535055/NID2 (+905); chr14:52535264-52535334/NID2 (+647); chr14:52536105-52536342/NID2 (−27
8); chr14:52735002-52735044/PTGDR (+592); chr14:52735424-52735451/PTGER2 (−45578), PTGDR (+1007);
chr14:53196654-53196689/STYX (−211); chr14:54413278-54413708/DDHD1 (−793447), BMP4 (+7777); chr14:5
4421801-54421831/BMP4 (−546); chr14:54686139-54686274/BMP4 (−264937), CDKN3 (−177466); chr14:55518
029-55518071/LGALS3 (−77885), SOCS4 (+24206); chr14:55518883-55518942/LGALS3 (−77022), SOCS4 (+2
5069); chr14:55738006-55738050/DLGAP5 (−79632), ATG14 (+140548); chr14:55738389-55738457/DLGAP5
(−80027), ATG14 (+140153); chr14:56047684-56047741/KTN1 (+788); chr14:57261095-57261174/OTX2 (+1604
9), C14orf101 (+214624); chr14:57261408-57261465/OTX2 (+15747), C14orf101 (+214926); chr14:57261880-5
7261965/OTX2 (+15261), C14orf101 (+215412); chr14:57264646-57264764/OTX2 (+12479), C14orf101 (+2181
94); chr14:57264867-57265005/OTX2 (+12248), C14orf101 (+218425); chr14:57265356-57265469/OTX2 (+117
71), C14orf101 (+218902); chr14:57265847-57265912/OTX2 (+11304), C14orf101 (+219369); chr14:57270972-
57271153/OTX2 (+6121), C14orf101 (+224552); chr14:57272114-57272210/OTX2 (+5022), C14orf101 (+22565
1); chr14:57275050-57275210/OTX2 (+2054), C14orf101 (+228619); chr14:57275686-57276056/OTX2 (+1313),
C14orf101 (+229360); chr14:57278763-57278837/OTX2 (−1616); chr14:57279565-57279642/OTX2 (−2420); chr
14:57283409-57283445/MUDENG (−452179), OTX2 (−6243); chr14:57284037-57284070/MUDENG (−451552),
OTX2 (−6870); chr14:58332022-58332055/SLC35F4 (+553); chr14:58332518-58332593/SLC35F4 (+36); chr14:
58332708-58332908/SLC35F4 (−216); chr14:58333098-58333161/SLC35F4 (−538); chr14:58893671-58893693/
KIAA0586 (−421), TIMM9 (+550); chr14:59104042-59104060/DACT1 (−706); chr14:59931009-59931023/GPR13
5 (+1043), DAAM1 (+275617); chr14:60386743-60386932/C14orf135 (−171791), RTN1 (−49281); chr14:609519
90-60952195/C14orf39 (+671); chr14:60952420-60952516/C14orf39 (+296); chr14:60952633-60952729/C14orf
39 (+83); chr14:60974008-60974077/SIX6 (−1895); chr14:60975889-60976074/SIX6 (+44); chr14:60976610-60
976802/SIX6 (+768); chr14:60977713-60977879/SIX6 (+1858), SIX1 (+138359); chr14:60981817-60981972/SI
X6 (+5957), SIX1 (+134260); chr14:60982368-60982573/SIX6 (+6533), SIX1 (+133684); chr14:61104155-6110
4241/SIX1 (+11957), SIX6 (+128260); chr14:61104557-61104623/SIX1 (+11565), SIX6 (+128652); chr14:61110
705-61110749/SIX1 (+5428), SIX6 (+134789); chr14:61118633-61118742/SIX1 (−2533); chr14:61747528-61747
582/TMEM30B (+975); chr14:62035389-62035430/HIF1A (−126709), PRKCH (+246895); chr14:62279476-6227
9519/SYT16 (−183043), SNAPC1 (+50423); chr14:62279853-62279898/SYT16 (−182665), SNAPC1 (+50801); c
hr14:62583870-62583918/SYT16 (+121353), KCNH5 (+928061); chr14:63512007-63512063/KCNH5 (−80); chr1
4:63512709-63512740/KCNH5 (−770); chr14:65006715-65006776/HSPA2 (−440); chr14:65007537-65007552/H
SPA2 (+359); chr14:65007692-65007714/HSPA2 (+517); chr14:65008975-65008997/PLEKHG3 (−185325), HS
PA2 (+1800); chr14:65172419-65172456/PLEKHG3 (−21873), HSPA2 (+165252); chr14:65438214-65438257/R
AB15 (+639); chr14:65453681-65453754/RAB15 (−14843), MAX (+115509); chr14:65879627-65879658/FUT8
(+2333); chr14:67878720-67878763/PLEK2 (+86); chr14:69074471-69074560/ZFP36L1 (+188444), RAD51B
(+788020); chr14:69095081-69095106/ZFP36L1 (+167866), RAD51B (+808598); chr14:69414543-69414668/ZFP
36L1 (−151646), ACTN1 (+31477); chr14:70038321-70038347/KIAA0247 (−39976), SLC39A9 (+172949); chr14:
70038590-70038620/KIAA0247 (−39705), SLC39A9 (+173220); chr14:70039623-70039665/KIAA0247 (−38666),
SLC39A9 (+174259); chr14:70655890-70655919/SLC8A3 (−118); chr14:71108228-71108278/TTC9 (−251); chr
14:71374938-71375021/PCNX (+858); chr14:72399246-72399269/RGS6 (+102); chr14:73181116-73181204/D
PF3 (+179649), RGS6 (+782004); chr14:73712377-73712419/PAPLN (+8193), NUMB (+212888); chr14:73712
709-73712888/PAPLN (+8594), NUMB (+212487); chr14:73924557-73924604/NUMB (+705); chr14:74035887-
74035929/ACOT2 (+136); chr14:74100886-74100906/ACOT6 (+17348), PNMA1 (+80232); chr14:74101033-74
101207/ACOT6 (+17572), PNMA1 (+80008); chr14:74179025-74179093/PNMA1 (+2069), ACOT6 (+95511); ch
r14:74226612-74226633/PNMA1 (−45495), C14orf43 (+27273); chr14:74253266-74253328/C14orf43 (+599); ch
r14:74485423-74485459/ENTPD5 (+585); chr14:74684579-74684656/ALDH6A1 (−133422), VSX2 (−21557); chr
14:74706397-74706940/VSX2 (+494); chr14:74707545-74707572/VSX2 (+1384), ABCD4 (+62208); chr14:7470
7748-74707791/VSX2 (+1595), ABCD4 (+61997); chr14:74892771-74892798/SYNDIG1L (+20); chr14:7496037
5-74960406/NPC2 (−307), ISCA2 (−59); chr14:74981388-74981475/ISCA2 (+20982), LTBP2 (+97602); chr14:75
077821-75077872/LTBP2 (+1187), ISCA2 (+117397); chr14:75078282-75078344/LTBP2 (+721); chr14:750784
64-75078492/LTBP2 (+556); chr14:75079514-75079555/LTBP2 (−501); chr14:75390071-75390106/RPS6KL1
(−901); chr14:75530648-75530703/ACYP1 (+60); chr14:75800460-75800598/JDP2 (−98308), FOS (+55048); chr1
4:75800988-75801052/JDP2 (−97817), FOS (+55539); chr14:75981561-75981708/BATF (−7149), JDP2 (+82798);
chr14:76445825-76445972/TGFB3 (+2193), TTLL5 (+318348); chr14:76449115-76449153/IFT43 (−2962), TG
FB3 (−1042); chr14:76843386-76843732/VASH1 (−384676), ESRRB (+5869); chr14:77192541-77192656/VASH
1 (−35636), ESRRB (+354909); chr14:77279251-77279272/VASH1 (+51027), IRF2BPL (+215772); chr14:77422
842-77422875/IRF2BPL (+72175), VASH1 (+194624); chr14:77495017-77495096/IRF2BPL (−23); chr14:77563
668-77563735/IRF2BPL (−68668), ZDHHC22 (+44432); chr14:77564173-77564235/IRF2BPL (−69170), ZDHHC
22 (+43930); chr14:77600216-77600264/IRF2BPL (−105206), ZDHHC22 (+7894); chr14:77606856-77606921/IR
F2BPL (−111855), ZDHHC22 (+1245); chr14:77736867-77736883/NGB (+780); chr14:77786644-77786699/GST
Z1 (−558), POMT2 (+553); chr14:77965219-77965244/ISM2 (−22); chr14:78083052-78083085/SPTLC2 (+41); c
hr14:78108064-78108264/SPTLC2 (−25054), ALKBH1 (+66192); chr14:78432776-78432795/NRXN3 (−437307),
ADCK1 (+166360); chr14:78447325-78447422/NRXN3 (−422719), ADCK1 (+180948); chr14:85996796-859968
91/FLRT2 (+356); chr14:85997926-85998186/FLRT2 (+1568); chr14:85998575-85998629/FLRT2 (+2114); chr1
4:85999532-85999596/FLRT2 (+3076); chr14:85999730-85999838/FLRT2 (+3296); chr14:88459127-88459170/
GALC (+466); chr14:88790247-88790298/KCNK10 (−680); chr14:90421019-90421129/TDP1 (−1172), EFCAB11
(+15); chr14:90849440-90849476/C14orf102 (−50977), CALM1 (−13869); chr14:90849858-90849897/C14orf102
(−51397), CALM1 (−13449); chr14:91225452-91225508/TTC7B (+57281), CALM1 (+362153); chr14:91287430-
91287542/TTC7B (−4725); chr14:91526648-91526680/RPS6KA5 (+329); chr14:91790478-91790620/GPR68 (−7
9697), CCDC88C (+93639); chr14:91883330-91883379/CCDC88C (+833); chr14:91883497-91883530/CCDC88
C (+674); chr14:91975849-91975897/SMEK1 (+771); chr14:91976332-91976383/SMEK1 (+286); chr14:925062
87-92506361/TRIP11 (+79); chr14:92588261-92588311/NDUFB1 (−133), CPSF2 (−12); chr14:92721338-927214
09/SLC24A4 (−68778), CPSF2 (+133076); chr14:92789945-92789953/SLC24A4 (−203); chr14:92790099-92790
280/SLC24A4 (+38); chr14:92980957-92980986/RIN3 (+847); chr14:93022097-93022189/RIN3 (+42018), LGM
N (+192904); chr14:93260359-93260403/GOLGA5 (−269); chr14:93389458-93389556/CHGA (+62); chr14:9338
9694-93389712/CHGA (+258); chr14:93896686-93896833/COX8C (+83223), PRIMA1 (+358006); chr14:93897
025-93897078/COX8C (+83515), PRIMA1 (+357714); chr14:94253958-94254150/PRIMA1 (+712); chr14:94254
459-94254498/PRIMA1 (+287); chr14:94255284-94255304/PRIMA1 (−528); chr14:94255607-94255669/PRIMA
1 (−872); chr14:94429210-94429343/PRIMA1 (−174511), ASB2 (+13799); chr14:94889871-94889915/SERPINA
1 (−32864), SERPINA11 (+29229); chr14:95234711-95235025/GSC (+1631), SERPINA13 (+127806); chr14:952
35383-95235513/GSC (+1051), SERPINA13 (+128386); chr14:95237643-95237782/GSC (−1214); chr14:95239
349-95239421/GSC (−2886); chr14:95657984-95658142/DICER1 (−49978), CLMN (+128182); chr14:95785544-
95785587/CLMN (+679); chr14:95826752-95826866/CLMN (−40564), C14orf49 (+115364); chr14:96000969-96
001007/GLRX5 (−335); chr14:96180491-96180642/TCL1A (−34); chr14:96180846-96180940/TCL1A (−360); chr1
4:96342310-96342530/C14orf132 (−163241), TCL1A (−161887); chr14:96342631-96342879/C14orf132 (−162906),
TCL1A (−162222); chr14:96343243-96343319/TCL1A (−162748), C14orf132 (−162380); chr14:96343505-9634
3666/TCL1A (−163053), C14orf132 (−162075); chr14:97058818-97058943/VRK1 (−204803), PAPOLA (+90168);
chr14:97059351-97059418/VRK1 (−204299), PAPOLA (+90672); chr14:97093419-97093503/VRK1 (−170223), P
APOLA (+124748); chr14:97499673-97499721/VRK1 (+236013); chr14:97499850-97499970/VRK1 (+236226);
chr14:97685297-97685395/VRK1 (+421662); chr14:98814597-98814691/BCL11B (+923178); chr14:99365922-
99366010/BCL11B (+371856); chr14:99584409-99584475/BCL11B (+153380); chr14:99697645-99697738/BCL
11B (+40130); chr14:99711874-99711967/BCL11B (+25901); chr14:99712087-99712133/BCL11B (+25712); ch
r14:99712245-99712286/BCL11B (+25556); chr14:99712635-99712819/BCL11B (+25095); chr14:99741213-99
741277/BCL11B (−3423); chr14:99947697-99947721/SETD3 (−483), CCNK (−30); chr14:100029881-100030008/
HHIPL1 (−81535), CCNK (+82206); chr14:100069714-100069898/HHIPL1 (−41674), CCNK (+122067); chr14:10
0126367-100126420/CYP46A1 (−24361), HHIPL1 (+14914); chr14:100126546-100126661/CYP46A1 (−24151),
HHIPL1 (+15124); chr14:100142252-100142348/CYP46A1 (−8455), HHIPL1 (+30820); chr14:100192657-10019
2715/EML1 (−67059), CYP46A1 (+41931); chr14:100218129-100218273/EML1 (−41544), CYP46A1 (+67446); c
hr14:100218694-100218710/EML1 (−41043), CYP46A1 (+67947); chr14:100259191-100259328/EML1 (−485); c
hr14:100437950-100438017/EVL (−93767), EML1 (+178239); chr14:100573334-100573426/EVL (+41629), DE
GS2 (+52632); chr14:100704776-100704793/YY1 (−317); chr14:100745092-100745135/SLC25A29 (+27746), Y
Y1 (+40012); chr14:100771141-100771173/SLC25A29 (+1703), YY1 (+66055); chr14:100771535-100771592/S
LC25A29 (+1296), YY1 (+66462); chr14:101012877-101012889/WARS (−170203), BEGAIN (+21524); chr14:10
1033393-101033483/BEGAIN (+969); chr14:101035718-101035730/BEGAIN (−1317); chr14:101069847-10106
9907/DLK1 (−123325), BEGAIN (−35470); chr14:101106694-101106721/DLK1 (−86494), BEGAIN (−72301); chr1
4:101180322-101180399/BEGAIN (−145954), DLK1 (−12841); chr14:101193147-101193345/DLK1 (+44); chr14:
101471994-101472082/DIO3 (−555650), RTL1 (−120854); chr14:101923804-101924029/RTL1 (−572733), DIO3
(−103771); chr14:101924244-101924295/RTL1 (−573086), DIO3 (−103418); chr14:101924438-101924457/RTL1
(−573264), DIO3 (−103240); chr14:101924792-101924857/RTL1 (−573641), DIO3 (−102863); chr14:101925457-1
01925482/RTL1 (−574286), DIO3 (−102218); chr14:101978623-101978672/RTL1 (−627464), DIO3 (−49040); chr
14:102027511-102027540/DIO3 (−162); chr14:102028125-102028144/DIO3 (+447); chr14:102031194-1020312
35/PPP2R5C (−196920), DIO3 (+3527); chr14:102031450-102031507/PPP2R5C (−196656), DIO3 (+3791); chr1
4:102031946-102032003/PPP2R5C (−196160), DIO3 (+4287); chr14:102172148-102172230/PPP2R5C (−55946),
DIO3 (+144501); chr14:102247524-102247551/DYNC1H1 (−183327), PPP2R5C (+19403); chr14:102829204-
102829226/TECPR2 (−85), CINP (+38); chr14:102975568-102975582/RCOR1 (−83421), TECPR2 (+146275); ch
r14:102976068-102976102/RCOR1 (−82911), TECPR2 (+146785); chr14:103058486-103058499/RCOR1 (−503);
chr14:103390033-103390096/AMN (+1072), CDC42BPB (+133677); chr14:103397030-103397068/AMN (+805
6), CDC42BPB (+126693); chr14:103568842-103568967/CDC42BPB (−45163), TNFAIP2 (−23759); chr14:10357
0396-103570455/CDC42BPB (−46684), TNFAIP2 (−22238); chr14:103603371-103603418/EIF5 (−197098), TNFA
IP2 (+10731); chr14:103605108-103605133/EIF5 (−195372), TNFAIP2 (+12457); chr14:103674021-103674032/
EIF5 (−126466), TNFAIP2 (+81363); chr14:103739631-103739754/EIF5 (−60800), TNFAIP2 (+147029); chr14:1
03740437-103740539/EIF5 (−60005), TNFAIP2 (+147824); chr14:103740855-103740890/EIF5 (−59620), TNFAI
P2 (+148209); chr14:103745238-103745292/EIF5 (−55228), TNFAIP2 (+152601); chr14:103745904-103745941/
EIF5 (−54570), TNFAIP2 (+153259); chr14:103746116-103746153/EIF5 (−54358), TNFAIP2 (+153471); chr14:1
03989529-103989537/CKB (−337); chr14:104028792-104028810/BAG5 (−146); chr14:104028958-104028974/B
AG5 (−311); chr14:104095389-104095455/KLC1 (−103); chr14:104096378-104096413/KLC1 (+871); chr14:1041
81581-104181640/ZFYVE21 (−470), XRCC3 (+212); chr14:104181750-104181773/ZFYVE21 (−319), XRCC3 (+6
1); chr14:104313692-104313720/PPP1R13B (+221); chr14:104552184-104552307/ASPG (+223); chr14:104583
036-104583110/KIF26A (−21987), ASPG (+31050); chr14:104601873-104601963/KIF26A (−3142); chr14:10461
6043-104616187/C14orf180 (−429941), KIF26A (+11055); chr14:104730078-104730212/C14orf180 (−315911),
KIF26A (+125085); chr14:104902674-104902726/C14orf180 (−143356), KIF26A (+297640); chr14:105155996-1
05156021/INF2 (+66); chr14:105189927-105189960/ADSSL1 (−6284), INF2 (+34001); chr14:105190853-10519
0900/ADSSL1 (−5351), INF2 (+34934); chr14:105202646-105202739/SIVA1 (−16777), ADSSL1 (+6465); chr14:
105267824-105267900/ZBTB42 (+929); chr14:105310443-105310485/KIAA0284 (−21186), ZBTB42 (+43531); c
hr14:105330940-105330980/KIAA0284 (−690); chr14:105331707-105331744/KIAA0284 (+76); chr14:10534591
3-105346018/PLD4 (−45221), KIAA0284 (+14316); chr14:105359303-105359363/PLD4 (−31854), KIAA0284 (+2
7683); chr14:105393143-105393211/PLD4 (+1990), AHNAK2 (+51517); chr14:105434160-105434286/AHNAK2
(+10471), PLD4 (+43036); chr14:105444634-105444788/AHNAK2 (−17); chr14:105527310-105527383/CDCA4
(−39922), GPR132 (+4407); chr14:105527490-105527543/CDCA4 (−40092), GPR132 (+4237); chr14:10555383
4-105553861/GPR132 (−22094), JAG2 (+81313); chr14:105649183-105649218/NUDT14 (−1541); chr14:105659
520-105659559/BTBD6 (−55339), NUDT14 (−11880); chr14:105714442-105714766/BTBD6 (−275); chr14:10571
4937-105715247/BTBD6 (+213); chr14:105715393-105715538/BTBD6 (+587); chr14:105767979-105768036/B
RF1 (−679); chr14:105780547-105780582/PACS2 (−187); chr14:105781311-105781365/PACS2 (+586); chr14:1
05781580-105781600/PACS2 (+838); chr14:105865018-105865048/TEX22 (+113); chr14:105880953-1058809
79/MTA1 (−5220), TEX22 (+16046); chr14:105888432-105888662/CRIP2 (−52584), MTA1 (+2361); chr14:10590
9329-105909435/CRIP2 (−31749), MTA1 (+23196); chr14:105919368-105919531/CRIP2 (−21681), MTA1 (+332
64); chr14:105930862-105930944/CRIP2 (−10228), MTA1 (+44717); chr14:105944243-105944421/CRIP1 (−892
5), CRIP2 (+3201); chr14:105944568-105944771/CRIP1 (−8587), CRIP2 (+3539); chr14:105952663-105952723/
CRIP1 (−564); chr14:105993486-105993517/TMEM121 (+549); chr14:105994980-105995121/TMEM121 (+209
8), IGHE (+334411); chr14:105995823-105995857/TMEM121 (+2887), IGHE (+333622); chr14:106146364-106
146394/TMEM121 (+153426), IGHE (+183083); chr14:107253106-107253323/ADAM6 (−814857); chr15:205001
81-20500202/HERC2P3 (+211241); chr15:22646691-22646737/GOLGA6L1 (−89532), OR4N3P (+233252); chr1
5:22647239-22647301/GOLGA6L1 (−88976), OR4N3P (+233808); chr15:23115150-23115175/GOLGA8IP (−140
079), NIPA1 (−28727); chr15:23164135-23164158/GOLGA8IP (−91095), NIPA1 (−77711); chr15:26107744-2610
7845/ATP10A (+554); chr15:26108327-26108548/ATP10A (−89); chr15:26327564-26327610/ATP10A (−219238),
GABRB3 (+690636); chr15:27016909-27016944/ATP10A (−908578), GABRB3 (+1296); chr15:27017211-2701
7361/GABRB3 (+937); chr15:27017524-27017586/GABRB3 (+668); chr15:27017947-27018117/GABRB3 (+191);
chr15:27018352-27018470/GABRB3 (−188); chr15:27112123-27112429/GABRA5 (+3); chr15:27112602-2711
2719/GABRA5 (+388); chr15:27112943-27113012/GABRA5 (+705); chr15:27113136-27113215/GABRA5 (+903);
chr15:27113410-27113477/GABRG3 (−102985), GABRA5 (+1171); chr15:27216276-27216304/GABRG3 (−13
9); chr15:27216491-27216830/GABRG3 (+232); chr15:27604124-27604144/GABRG3 (+387705), OCA2 (+7403
24); chr15:28339933-28339954/OCA2 (+4514); chr15:28342116-28342124/OCA2 (+2338); chr15:28342255-28
342521/OCA2 (+2070); chr15:28344188-28344223/OCA2 (+252); chr15:28344350-28344411/OCA2 (+77); chr1
5:28352121-28352152/OCA2 (−7679), HERC2 (+215158); chr15:28352422-28352607/OCA2 (−8057), HERC2
(+214780); chr15:28352737-28352792/OCA2 (−8307), HERC2 (+214530); chr15:28352905-28352957/OCA2 (−84
73), HERC2 (+214364); chr15:28367156-28367210/OCA2 (−22725), HERC2 (+200112); chr15:28648073-28648
379/APBA2 (−565617), HERC2 (−80931); chr15:28700335-28700373/APBA2 (−513489), HERC2 (−133059); chr1
5:29033892-29033933/HERC2 (−466618), APBA2 (−179930); chr15:29034272-29034292/HERC2 (−466987), AP
BA2 (−179561); chr15:29034493-29034565/HERC2 (−467234), APBA2 (−179314); chr15:29077394-29077428/H
ERC2 (−510116), APBA2 (−136432); chr15:29077624-29077646/HERC2 (−510340), APBA2 (−136208); chr15:29
131314-29131356/HERC2 (−564040), APBA2 (−82508); chr15:29131631-29131755/HERC2 (−564398), APBA2
(−82150); chr15:29395858-29396025/NDNL2 (+166078), APBA2 (+182099); chr15:29396215-29396275/NDNL2
(+165775), APBA2 (+182402); chr15:29407814-29407866/NDNL2 (+154180), APBA2 (+193997); chr15:299672
36-29967275/FAM189A1 (−104329), TJP1 (+147450); chr15:29967395-29967587/FAM189A1 (−104564), TJP1
(+147215); chr15:30114303-30114349/TJP1 (+380); chr15:30515184-30515201/TJP1 (−400487), CHRFAM7A
(+170671); chr15:30515409-30515438/TJP1 (−400718), CHRFAM7A (+170440); chr15:30515563-30515630/TJP1
(−400891), CHRFAM7A (+170267); chr15:30517227-30517249/TJP1 (−402532), CHRFAM7A (+168626); chr15:
30517525-30517600/TJP1 (−402857), CHRFAM7A (+168301); chr15:30517994-30518134/TJP1 (−403358), CH
RFAM7A (+167800); chr15:30918971-30919019/ARHGAP11B (+116); chr15:31620491-31620530/KLF13 (+142
8), OTUD7A (+327031); chr15:31620744-31620800/KLF13 (+1689), OTUD7A (+326770); chr15:31727006-317
27135/KLF13 (+107988), OTUD7A (+220471); chr15:31775474-31775483/KLF13 (+156396), OTUD7A (+17206
3); chr15:31775638-31775678/KLF13 (+156575), OTUD7A (+171884); chr15:31775862-31775976/KLF13 (+156
836), OTUD7A (+171623); chr15:31776190-31776278/KLF13 (+157151), OTUD7A (+171308); chr15:32605000-
32605081/ARHGAP11A (−302650), CHRNA7 (+282350); chr15:32605305-32605350/ARHGAP11A (−302363),
CHRNA7 (+282637); chr15:32605490-32605507/ARHGAP11A (−302192), CHRNA7 (+282808); chr15:32607591-
32607849/ARHGAP11A (−299971), CHRNA7 (+285029); chr15:32634605-32634655/ARHGAP11A (−273061),
CHRNA7 (+311939); chr15:33009560-33009613/GREM1 (−618); chr15:33009762-33009821/GREM1 (−413); chr
15:33010676-33010720/GREM1 (+493); chr15:33360261-33360336/FMN1 (−214); chr15:33603609-33603647/
RYR3 (+451); chr15:33603790-33603879/RYR3 (+658); chr15:34630389-34630438/SLC12A6 (−1369); chr15:34
635079-34635126/C15orf55 (−2963), NOP10 (+259); chr15:34728789-34728813/GOLGA8A (−46800), GOLGA8
B (+99421); chr15:34786944-34787232/GOLGA8A (−105087), GOLGA8B (+41134); chr15:34787473-34787585/
GOLGA8A (−105528), GOLGA8B (+40693); chr15:34806445-34806507/GOLGA8A (−124475), GOLGA8B (+21
746); chr15:34874692-34874747/GOLGA8B (−46498), GJD2 (+171969); chr15:35046609-35046689/GJD2 (+40);
chr15:35047149-35047293/GJD2 (−532); chr15:35261632-35261697/AQR (+330); chr15:35965359-35965432/
ZNF770 (−684899); chr15:37172589-37172688/MEIS2 (+220102); chr15:37172792-37172923/MEIS2 (+219883);
chr15:37389363-37389455/MEIS2 (+3332); chr15:37390175-37390198/MEIS2 (+2554); chr15:37390316-373
90339/MEIS2 (+2413); chr15:37393117-37393160/MEIS2 (−398); chr15:37395784-37395913/MEIS2 (−3108); ch
r15:37402546-37402645/TMCO5A (−824862), MEIS2 (−9855); chr15:37403087-37403115/TMCO5A (−824357),
MEIS2 (−10360); chr15:39873303-39873365/THBS1 (+54); chr15:40226253-40226266/EIF2AK4 (−87); chr15:40
400490-40400526/BMF (+567); chr15:40650450-40650479/DISP2 (+31); chr15:40650961-40651001/DISP2 (+5
47); chr15:40660111-40660143/C15orf23 (−14795), DISP2 (+9693); chr15:40675028-40675055/C15orf23 (+120);
chr15:40729185-40729292/BAHD1 (−4172); chr15:41046920-41046991/FAM82A2 (+502); chr15:41047236-4
1047314/FAM82A2 (+183); chr15:41218030-41218093/DLL4 (−3469); chr15:41218220-41218245/DLL4 (−3298);
chr15:41234093-41234122/CHAC1 (−11528), DLL4 (+12577); chr15:41624420-41624463/NUSAP1 (−450), OIP
5 (+377); chr15:41694458-41694512/NDUFAF1 (+173); chr15:41788138-41788173/ITPKA (+2100), LTK (+1792
9); chr15:41793581-41793873/ITPKA (+7671), LTK (+12358); chr15:41794817-41795239/ITPKA (+8972), LTK
(+11057); chr15:41795691-41795714/ITPKA (+9647), LTK (+10382); chr15:41804022-41804115/LTK (+2016), I
TPKA (+18013); chr15:41851447-41851459/TYRO3 (+233); chr15:42264697-42264728/EHD4 (+42); chr15:425
65605-42565650/GANC (−738), TMEM87A (+127); chr15:43415198-43415273/UBR1 (−16950), TMEM62 (−1048
6); chr15:43803760-43803825/TP53BP1 (−18439), MAP1A (−6013); chr15:44084925-44084979/SERF2 (+778);
chr15:45315349-45315418/SORD (+82); chr15:45328132-45328270/SORD (+12899), DUOX2 (+78158); chr15:
45403214-45403337/DUOXA2 (−3247), DUOX2 (+3083); chr15:45403681-45403798/DUOXA2 (−2783), DUOX2
(+2619); chr15:45404000-45404102/DUOXA2 (−2472), DUOX2 (+2308); chr15:45404833-45404897/DUOXA2
(−1658), DUOX2 (+1494); chr15:45409372-45409491/DUOX2 (−3073), DUOXA2 (+2909); chr15:45421681-45421
706/DUOX1 (−498), DUOXA1 (+363); chr15:45421950-45421997/DUOX1 (−218), DUOXA1 (+83); chr15:454273
41-45427369/DUOX1 (+5163), SHF (+66018); chr15:45427720-45427771/DUOX1 (+5554), SHF (+65627); chr1
5:45427913-45428307/DUOX1 (+5918), SHF (+65263); chr15:45670130-45670188/GATM (+821); chr15:45670
839-45670932/GATM (+94); chr15:45694530-45694556/SPATA5L1 (+24); chr15:45996563-45996725/SQRDL
(+69388); chr15:45997063-45997089/SQRDL (+69820); chr15:47476240-47476290/SEMA6D (−534421); chr15:
47476420-47476468/SEMA6D (−534242); chr15:47476806-47476876/SEMA6D (−533845); chr15:47476981-474
77012/SEMA6D (−533689); chr15:47477265-47477354/SEMA6D (−533376); chr15:48470146-48470215/MYEF2
(+377); chr15:48483758-48483906/CTXN2 (−35); chr15:48624362-48624496/DUT (+808); chr15:48937646-489
37709/FBN1 (+307); chr15:50646612-50646641/GABPB1 (+978); chr15:51634151-51634174/CYP19A1 (−3368),
GLDN (+450); chr15:51973695-51973763/SCG3 (+179); chr15:52264427-52264479/LEO1 (−495); chr15:5247
1661-52471816/BCL2L10 (−66767), GNB5 (+11826); chr15:52546480-52546599/GNB5 (−62975), MYO5C (+414
55); chr15:53072801-53072851/ARPP19 (−211613), ONECUT1 (+9383); chr15:53075660-53075739/ARPP19
(−214487), ONECUT1 (+6509); chr15:53075865-53075985/ARPP19 (−214712), ONECUT1 (+6284); chr15:53077
001-53077065/ARPP19 (−215820), ONECUT1 (+5176); chr15:53079678-53079717/ARPP19 (−218485), ONECU
T1 (+2511); chr15:53079892-53079970/ARPP19 (−218718), ONECUT1 (+2278); chr15:53080620-53080736/AR
PP19 (−219465), ONECUT1 (+1531); chr15:53080991-53081054/ARPP19 (−219810), ONECUT1 (+1186); chr15:
53081297-53081398/ONECUT1 (+861); chr15:53082021-53082144/ONECUT1 (+126); chr15:53083043-53083
116/ONECUT1 (−871); chr15:53083485-53083653/ONECUT1 (−1360); chr15:53086967-53087075/ONECUT1
(−4812); chr15:53087376-53087451/ONECUT1 (−5205); chr15:53090272-53090305/ONECUT1 (−8080); chr15:53
093151-53093342/ONECUT1 (−11038); chr15:53097907-53098381/ONECUT1 (−15935); chr15:55581873-5558
1886/PIGB (−29253), RAB27A (−18773); chr15:55880783-55880890/PYGO1 (+213); chr15:56034723-56034885/
PRTG (+373); chr15:56035260-56035304/PRTG (−105); chr15:58357402-58357799/ALDH1A2 (+520); chr15:5
8358473-58358503/ALDH1A2 (−367); chr15:59042071-59042128/ADAM10 (+77); chr15:59397038-59397082/C
CNB2 (−224); chr15:59949123-59949186/GTF2A2 (+582); chr15:59949616-59949696/GTF2A2 (+81); chr15:602
87586-60287643/BNIP2 (−305973), FOXB1 (−8806); chr15:60296632-60296860/FOXB1 (+325); chr15:6029782
7-60297941/FOXB1 (+1463), ANXA2 (+392301); chr15:61520555-61520576/RORA (−600837), VPS13C (+8320
98); chr15:61521294-61521311/RORA (−601574), VPS13C (+831361); chr15:61521621-61521656/RORA (−601
910), VPS13C (+831025); chr15:62360146-62360177/C2CD4A (+986); chr15:62456966-62457008/C2CD4B
(+495); chr15:63673604-63673652/CA12 (+447); chr15:63893466-63893529/USP3 (+96688), HERC1 (+232649);
chr15:64125910-64125924/HERC1 (+230); chr15:64454945-64455005/PPIB (+379); chr15:65067395-6506744
5/RBPMS2 (+350); chr15:65116156-65116216/RBPMS2 (−48416), PIF1 (+1652); chr15:65133693-65133806/A
NKDD1A (−70351), PIF1 (−15912); chr15:65341696-65341754/OSTBETA (+4017), RASL12 (+18663); chr15:653
60293-65360323/RASL12 (+80); chr15:65370301-65370340/KBTBD13 (+1167), PDCD7 (+55853); chr15:65370
516-65370564/KBTBD13 (+1386), PDCD7 (+55634); chr15:65477608-65477663/CLPX (−73); chr15:65648100-6
5648136/PARP16 (−69100), IGDCC3 (+22260); chr15:65689099-65689348/IGDCC3 (−18846), IGDCC4 (+26186);
chr15:65715493-65715513/IGDCC4 (−93); chr15:66585667-66585725/DIS3L (−224); chr15:66648575-666486
23/TIPIN (+455); chr15:66914719-66914742/SMAD6 (−79943), LCTL (−56896); chr15:67055825-67055897/SMA
D3 (−302334), SMAD6 (+61187); chr15:67135023-67135059/SMAD3 (−223154), SMAD6 (+140367); chr15:6732
5908-67325991/SMAD3 (−32245), SMAD6 (+331276); chr15:67357533-67357544/SMAD3 (−656); chr15:673583
53-67358377/SMAD3 (+170); chr15:67834900-67834962/MAP2K5 (−90); chr15:68111967-68112086/SKOR1
(−5914), MAP2K5 (+277006); chr15:68112227-68112276/SKOR1 (−5689), MAP2K5 (+277231); chr15:68112440-6
8112498/SKOR1 (−5472), MAP2K5 (+277448); chr15:68118876-68118929/SKOR1 (+962); chr15:68119428-681
19455/PIAS1 (−227130), SKOR1 (+1501); chr15:68120358-68120630/PIAS1 (−226078), SKOR1 (+2553); chr15:
68120959-68121149/PIAS1 (−225518), SKOR1 (+3113); chr15:68121958-68122286/PIAS1 (−224450), SKOR1
(+4181); chr15:68122405-68122457/PIAS1 (−224141), SKOR1 (+4490); chr15:68724442-68724480/ITGA11 (+3
1); chr15:68851469-68851534/ITGA11 (−127010), CORO2B (−57377); chr15:68869973-68870013/ITGA11 (−145
501), CORO2B (−38886); chr15:69087721-69087781/ANP32A (+25510), CORO2B (+178872); chr15:69111702-
69111754/ANP32A (+1533), CORO2B (+202849); chr15:69706455-69706516/KIF23 (−141); chr15:70641356-70
641447/TLE3 (−251146), UACA (+414448); chr15:71146313-71146345/LARP6 (+169); chr15:71407483-714075
37/THSD4 (−26278), LRRC49 (+222582); chr15:72766579-72766608/ARIH1 (−73); chr15:72922664-72922725/B
BS4 (−55825), ARIH1 (+156028); chr15:72978450-72978506/BBS4 (−42); chr15:73660327-73660446/HCN4 (+1
218), NEO1 (+315562); chr15:73660724-73660793/HCN4 (+846); chr15:73925010-73925058/NPTN (+719); chr
15:74044450-74044487/TBC1D21 (−121499), CD276 (+67847); chr15:74045023-74045074/TBC1D21 (−120919),
CD276 (+68427); chr15:74134497-74134581/TBC1D21 (−31429), CD276 (+157917); chr15:74219213-742192
93/LOXL1 (+464); chr15:74284537-74284589/PML (−2451), STOML1 (+72); chr15:74421954-74421979/ISLR2
(−776); chr15:74425566-74426180/ISLR (−41032), ISLR2 (+3130); chr15:74426304-74426762/ISLR (−40372), IS
LR2 (+3790); chr15:74426956-74427056/ISLR (−39899), ISLR2 (+4263); chr15:74428683-74428716/ISLR (−382
05), ISLR2 (+5957); chr15:74429616-74429721/ISLR (−37236), ISLR2 (+6926); chr15:74658397-74658501/CYP
11A1 (+1632), CCDC33 (+129782); chr15:74725503-74725548/SEMA7A (+773); chr15:74988350-74988381/E
DC3 (+20); chr15:75018265-75018311/CYP1A1 (−411); chr15:75251414-75251478/RPP25 (−1671); chr15:7547
0911-75471217/DNM1P34 (+124242), PPCDC (+155137); chr15:75640496-75640520/NEIL1 (+1177), MAN2C1
(+20433); chr15:75660927-75660967/MAN2C1 (−6); chr15:75743154-75743204/SIN3A (+747); chr15:7574747
6-75747524/SIN3A (−3574); chr15:75939641-75939671/SNX33 (−1692); chr15:76351998-76352038/C15orf27
(−281); chr15:76627537-76627575/ISL2 (−1591); chr15:76630125-76630513/ISL2 (+1172), SCAPER (+545898); c
hr15:76632146-76632517/ISL2 (+3185), SCAPER (+543885); chr15:76633170-76633206/ISL2 (+4041), SCAPE
R (+543029); chr15:76633985-76634013/ISL2 (+4852), SCAPER (+542218); chr15:76634812-76634842/ISL2
(+5680), SCAPER (+541390); chr15:76635168-76635504/ISL2 (+6189), SCAPER (+540881); chr15:76638571-7
6638609/ISL2 (+9443), SCAPER (+537627); chr15:76639545-76639720/ISL2 (+10486), SCAPER (+536584); c
hr15:77197619-77197683/RCN2 (−26311), SCAPER (−21434); chr15:77288814-77288855/PSTPIP1 (+1370), T
SPAN3 (+74735); chr15:78111120-78111195/LINGO1 (−186449), TBC1D2B (+258836); chr15:78111869-78111
916/LINGO1 (−187184), TBC1D2B (+258101); chr15:78591578-78591643/WDR61 (+329); chr15:78632669-786
32683/CRABP1 (+10); chr15:78730795-78730830/IREB2 (+295); chr15:78730945-78730984/IREB2 (+447); chr
15:78912372-78912441/CHRNA3 (+1230), CHRNA5 (+54545); chr15:78913028-78913094/CHRNA3 (+576); ch
r15:78913470-78913480/CHRNA3 (+162); chr15:79123257-79123337/MORF4L1 (−41875), ADAMTS7 (−19524);
chr15:79237097-79237144/CTSH (+299); chr15:79381746-79382098/CTSH (−144502), RASGRF1 (+1293); chr
15:79383384-79383438/RASGRF1 (−196); chr15:79724120-79724351/KIAA1024 (−622); chr15:79724561-7972
4606/KIAA1024 (−274); chr15:79724793-79724863/KIAA1024 (−30); chr15:80189019-80189066/MTHFS (+348);
chr15:80189531-80189549/MTHFS (−149); chr15:80445425-80445463/FAH (+211); chr15:80544336-80544364/
ARNT2 (−152342), FAH (+99117); chr15:81426601-81426681/MESDC2 (−144436), IL16 (−90999); chr15:82337
728-82337802/MEX3B (+596); chr15:82555204-82555281/EFTUD1 (−139); chr15:83315614-83315638/RPS17L
(−106418), CPEB1 (+1102); chr15:83315990-83316231/CPEB1 (+617); chr15:83348845-83348912/CPEB1 (−32
151), AP3B2 (+29756); chr15:83349612-83349671/CPEB1 (−32914), AP3B2 (+28993); chr15:83775849-837758
90/TM6SF1 (−454); chr15:83776002-83776022/TM6SF1 (−312); chr15:83776138-83776157/TM6SF1 (−176); chr
15:83776307-83776332/TM6SF1 (−4); chr15:83776717-83776768/TM6SF1 (+419); chr15:83777020-83777097/
TM6SF1 (+735); chr15:83875666-83875705/HDGFRP3 (+635); chr15:83876083-83876201/HDGFRP3 (+179); c
hr15:83876483-83876654/HDGFRP3 (−248); chr15:83952124-83952768/HDGFRP3 (−76125), BNC1 (+1022); c
hr15:83953117-83953303/BNC1 (+258); chr15:83953410-83953486/BNC1 (+20); chr15:84115854-84115931/S
H3GL3 (−198); chr15:84116339-84116377/SH3GL3 (+267); chr15:84116553-84116651/SH3GL3 (+511); chr15:
84116764-84116828/SH3GL3 (+705); chr15:84322322-84322367/ADAMTSL3 (−493); chr15:84322860-8432288
3/ADAMTSL3 (+34); chr15:84323261-84323372/ADAMTSL3 (+479); chr15:84748620-84748678/ZSCAN2 (−395
600), ADAMTSL3 (+425811); chr15:85144626-85144641/ZSCAN2 (+385); chr15:85201465-85201502/NMB (+3
18); chr15:85360718-85360760/ALPK3 (+828); chr15:85923271-85923324/AKAP13 (−573); chr15:85923546-85
923617/AKAP13 (−289); chr15:86337720-86337757/KLHL25 (+450); chr15:88798321-88798364/NTRK3 (+1619);
chr15:88798654-88798724/NTRK3 (+1273); chr15:88798989-88799036/NTRK3 (+949); chr15:88800302-888
00412/NTRK3 (−395); chr15:88801009-88801088/NTRK3 (−1087); chr15:89147680-89147722/DET1 (−57789), A
EN (−16826); chr15:89312771-89312794/ACAN (−33891), ISG20 (+130744); chr15:89313061-89313078/ACAN
(−33604), ISG20 (+131031); chr15:89346327-89346346/ACAN (−337); chr15:89346794-89346881/ACAN (+164);
chr15:89631117-89631175/ABHD2 (−235); chr15:89877857-89877872/POLG (+161); chr15:89914041-8991413
5/POLG (−36062), RHCG (+125711); chr15:89914450-89914866/POLG (−36632), RHCG (+125141); chr15:8992
0706-89920968/POLG (−42811), RHCG (+118962); chr15:89921112-89921167/POLG (−43114), RHCG (+11865
9); chr15:89922226-89922323/POLG (−44249), RHCG (+117524); chr15:89943775-89943816/POLG (−65770),
RHCG (+96003); chr15:89949410-89949616/POLG (−71487), RHCG (+90286); chr15:89950897-89950961/POL
G (−72903), RHCG (+88870); chr15:89951377-89951465/POLG (−73395), RHCG (+88378); chr15:89952453-89
952563/POLG (−74482), RHCG (+87291); chr15:89956354-89956422/POLG (−78362), RHCG (+83411); chr15:8
9973850-89973912/POLG (−95855), RHCG (+65918); chr15:90118614-90118695/C15orf42 (−163); chr15:90197
992-90198066/KIF7 (+653); chr15:90294070-90294092/MESP1 (+459); chr15:90320013-90320089/MESP2 (+4
62); chr15:90357590-90357681/ANPEP (+436); chr15:90358060-90358102/ANPEP (−9); chr15:90358482-9035
8536/ANPEP (−437); chr15:90745210-90745254/SEMA4B (+670); chr15:90774476-90774544/C15orf58 (−2977),
CIB1 (+2769); chr15:90931324-90931437/IQGAP1 (−92); chr15:91478168-91478242/HDDC3 (−2431), UNC45A
(−10); chr15:92937654-92937696/ST8SIA2 (+535); chr15:92938061-92938122/ST8SIA2 (+952); chr15:9293829
4-92938315/ST8SIA2 (+1165), FAM174B (+260726); chr15:93277268-93277366/CHD2 (−166234), FAM174B
(−78286); chr15:93592315-93592359/RGMA (+24879), CHD2 (+148786); chr15:93616379-93616396/RGMA (+82
8); chr15:93616893-93616958/RGMA (+290); chr15:93631396-93631632/RGMA (−14298); chr15:93823455-938
23555/RGMA (−206289); chr15:96596276-96596317/NR2F2 (−277814); chr15:96877328-96877631/NR2F2 (+33
69); chr15:96884911-96884955/NR2F2 (+10822); chr15:96902737-96902957/NR2F2 (+28736); chr15:9690945
4-96909526/NR2F2 (+35379); chr15:96911692-96911763/NR2F2 (+37617); chr15:96912815-96912957/NR2F2
(+38775); chr15:96951691-96951731/NR2F2 (+77600); chr15:96953099-96953131/NR2F2 (+79004); chr15:969
59549-96959719/NR2F2 (+85523); chr15:96959961-96960189/NR2F2 (+85964); chr15:98503555-98503609/A
RRDC4 (−351); chr15:98503877-98503946/ARRDC4 (−21); chr15:99190403-99190462/IGF1R (−2328); chr15:99
191024-99191091/IGF1R (−1703); chr15:99191726-99191748/IGF1R (−1024); chr15:99601915-99601980/PGP
EP1L (−53063), SYNM (−43338); chr15:99602250-99602275/PGPEP1L (−53378), SYNM (−43023); chr15:996406
97-99640745/SYNM (−4565); chr15:99993063-99993116/TTC23 (−203275), MEF2A (−113043); chr15:10034673
6-100346749/LYSMD4 (−73117), ADAMTS17 (+535440); chr15:100523267-100523359/LYSMD4 (−249687), AD
AMTS17 (+358870); chr15:100524300-100524410/LYSMD4 (−250729), ADAMTS17 (+357828); chr15:1008826
21-100882681/ADAMTS17 (−468); chr15:100913596-100914039/ADAMTS17 (−31635), CERS3 (+171107); chr1
5:101142231-101142301/ASB7 (−489); chr15:101319326-101319426/ALDH1A3 (−100633), ASB7 (+176621); ch
r15:101419261-101419292/ALDH1A3 (−732); chr15:101420384-101420425/ALDH1A3 (+396); chr15:101420665-
101420734/ALDH1A3 (+691); chr15:101420860-101420971/ALDH1A3 (+907); chr15:101513366-101513700/L
RRK1 (+54073), CHSY1 (+278604); chr15:101514020-101514085/LRRK1 (+54593), CHSY1 (+278084); chr15:
101662478-101662585/CHSY1 (+129605), LRRK1 (+203072); chr15:101991885-101991946/SNRPA1 (−156456),
PCSK6 (+38271); chr15:102036977-102037061/PCSK6 (−6832), TM2D3 (+155575); chr15:102192517-10219
2543/TM2D3 (+64); chr15:102264298-102264338/TARSL2 (+327); chr15:102285731-102285762/OR4F6 (−6017
6), TARSL2 (−21102); chr15:102286360-102286450/OR4F6 (−59518), TARSL2 (−21760); chr16:103714-103749/
POLR3K (−100), SNRNP25 (−97); chr16:204442-204480/HBM (−11512), HBZ (+1607); chr16:215873-215912/HB
M (−80); chr16:216447-216505/HBM (+503); chr16:230450-230496/HBQ1 (+140); chr16:285606-285643/ITFG3
(+824); chr16:374816-374858/AXIN1 (+27839), PDIA2 (+42222); chr16:375198-375306/AXIN1 (+27424), PDIA
2 (+42637); chr16:420585-420714/MRPL28 (−81); chr16:596925-596963/PIGQ (−23024), SOLH (+19088); chr16:
610316-610343/PIGQ (−9638), SOLH (+32474); chr16:616826-616856/PIGQ (−3127); chr16:729642-729700/ST
UB1 (−444); chr16:776949-777020/HAGHL (−281); chr16:838230-838262/CHTF18 (−376), RPUSD1 (+137); chr1
6:880232-880330/GNG13 (−29548), LMF1 (+140703); chr16:881319-881414/GNG13 (−30634), LMF1 (+139617);
chr16:1021077-1021119/LMF1 (−114); chr16:1030271-1030443/SOX8 (−1451); chr16:1030886-1030920/SOX8
(−905); chr16:1032621-1032660/SOX8 (+833); chr16:1115679-1115884/SSTR5 (−6974), SOX8 (+83974); chr16:
1122947-1123001/SSTR5 (+218); chr16:1202433-1202546/CACNA1H (−751); chr16:1203109-1203151/CACN
A1H (−111); chr16:1203784-1203807/CACNA1H (+555); chr16:1203963-1204002/CACNA1H (+742); chr16:120
4111-1204128/CACNA1H (+879); chr16:1295083-1295106/TPSD1 (−11178), TPSAB1 (+4417); chr16:1383986-
1384020/BAIAP3 (−502); chr16:1429455-1429495/GNPTG (+27575), UNKL (+35230); chr16:1464133-1464197/
UNKL (+540); chr16:1464400-1464441/UNKL (+284); chr16:1832093-1832161/NUBP2 (−806), SPSB3 (+454); c
hr16:1877289-1877332/HAGH (−116), FAHD1 (+86); chr16:1922305-1922421/C16orf73 (−184); chr16:2009771-
2009877/NDUFB10 (+307); chr16:2010101-2010163/NDUFB10 (+615); chr16:2014592-2014613/RNF151 (−227
2), RPS2 (+224); chr16:2029706-2029783/GFER (−4405), NOXO1 (+1695); chr16:2040961-2040980/SYNGR3
(+1025), ZNF598 (+18792); chr16:2041513-2041581/SYNGR3 (+1601), ZNF598 (+18216); chr16:2042599-2042
617/SYNGR3 (+2662), ZNF598 (+17155); chr16:2058088-2058242/ZNF598 (+1598), SYNGR3 (+18219); chr16:
2058908-2058953/ZNF598 (+832); chr16:2070108-2070147/NPW (+607); chr16:2228878-2229056/CASKIN1
(+17498), TRAF7 (+23168); chr16:2246321-2246394/CASKIN1 (+107); chr16:2255453-2255473/MLST8 (+285); c
hr16:2255672-2255773/MLST8 (+545); chr16:2256686-2256819/MLST8 (+1575), C16orf79 (+4316); chr16:228
2243-2282326/DNASE1L2 (−4183); chr16:2286867-2286954/DNASE1L2 (+443); chr16:2287176-2287230/DNA
SE1L2 (+735); chr16:2317567-2317602/RNPS1 (+273); chr16:2317714-2317877/RNPS1 (+62); chr16:2391719-
2391769/ABCA3 (−997); chr16:2392043-2392126/ABCA3 (−1338); chr16:2509928-2509956/NTN3 (−11558), CC
NF (+30547); chr16:2523639-2523707/TBC1D24 (−1474); chr16:2557412-2557487/ATP6V0C (−6277), TBC1D2
4 (+32303); chr16:2587853-2587880/PDPK1 (−103); chr16:2892603-2892626/PRSS30P (−142); chr16:2892797-
2892911/PRSS30P (−381); chr16:2902857-2902886/PRSS30P (−10399), PRSS22 (+5299); chr16:2933591-293
3612/FLYWCH2 (+406); chr16:2954365-2954428/FLYWCH1 (−7583), FLYWCH2 (+21201); chr16:3011021-301
1149/KREMEN2 (−3132); chr16:3016539-3016646/PAQR4 (−2749); chr16:3016850-3017156/PAQR4 (−2339); ch
r16:3017435-3017733/PAQR4 (−1758); chr16:3030667-3030710/PKMYT1 (−149); chr16:3078150-3078252/HCF
C1R1 (−3914); chr16:3096789-3096821/MMP25 (+123); chr16:3156802-3156830/ZSCAN10 (−13955), ZNF205
(−5747); chr16:3174434-3174491/ZNF213 (−10594), ZNF205 (+11900); chr16:3184826-3184880/ZNF213 (−204);
chr16:3209374-3209411/OR1F1 (−44854), CASP14L (+15173); chr16:3220287-3220438/OR1F1 (−33884), CAS
P14L (+26143); chr16:3220557-3220590/OR1F1 (−33673), CASP14L (+26354); chr16:3220893-3221141/OR1F
1 (−33230), CASP14L (+26797); chr16:3221701-3221786/OR1F1 (−32503), CASP14L (+27524); chr16:3233017-
3233198/OR1F1 (−21139), CASP14L (+38888); chr16:3233331-3233434/OR1F1 (−20864), CASP14L (+39163);
chr16:3234104-3234164/OR1F1 (−20113), CASP14L (+39914); chr16:3355234-3355250/ZNF75A (−191), TIGD7
(+197); chr16:3508054-3508088/NAA60 (+79); chr16:3508197-3508211/NAA60 (+212); chr16:3550079-35501
17/CLUAP1 (−865); chr16:3630428-3630517/NLRC3 (−3081); chr16:3929428-3929483/CREBBP (+665); chr16:4
165376-4165448/ADCY9 (+774); chr16:4234348-4234380/ADCY9 (−68178), SRL (+57717); chr16:4290102-429
0219/ADCY9 (−123975), SRL (+1920); chr16:4343850-4343938/GLIS2 (−38322), TFAP4 (−20893); chr16:436640
5-4366415/TFAP4 (−43409), GLIS2 (−15806); chr16:4377508-4377552/GLIS2 (−4686); chr16:4524583-4524619/
HMOX2 (−1740), NMRAL1 (+295); chr16:4588089-4588308/C16orf5 (+617); chr16:4744272-4744315/NUDT16L
1 (+600); chr16:4956642-4956744/PPL (+30443), UBN1 (+58781); chr16:5290747-5290791/ALG1 (+168959); c
hr16:5540579-5540672/ALG1 (+418816); chr16:6068927-6068936/ALG1 (+947122); chr16:6069056-6069094/A
LG1 (+947265); chr16:6069197-6069249/ALG1 (+947413); chr16:6069552-6069577/ALG1 (+947755); chr16:60
69685-6069940/ALG1 (+948003); chr16:6070454-6070542/ALG1 (+948688); chr16:6533295-6533425/RBFOX1
(−849391); chr16:6533898-6533913/RBFOX1 (−848845); chr16:6623776-6623842/RBFOX1 (−758942); chr16:7
353408-7353471/RBFOX1 (−29311); chr16:7354040-7354077/RBFOX1 (−28692); chr16:7354658-7354699/RBF
OX1 (−28072); chr16:9185071-9185102/USP7 (−127746); chr16:10277051-10277071/GRIN2A (−798); chr16:109
12268-10912299/FAM18A (+337); chr16:10912434-10912508/FAM18A (+150); chr16:11036128-11036153/CIIT
A (+65086), SOCS1 (+313898); chr16:11038164-11038215/CIITA (+67135), SOCS1 (+311849); chr16:1132701
5-11327283/SOCS1 (+22890), CIITA (+356094); chr16:11367141-11367181/TNP2 (−4001), PRM3 (+291); chr1
6:11439091-11439156/RMI2 (−187); chr16:11680895-11680924/LITAF (−104); chr16:11761822-11761887/SNN
(−434); chr16:11836628-11836647/TXNDC11 (+10); chr16:12009160-12009187/GSPT1 (+651); chr16:1299583
9-12996045/SHISA9 (+465); chr16:14164966-14165019/MKL2 (−203); chr16:14397594-14397850/MKL2 (+2325
26), PARN (+326406); chr16:14403370-14403424/MKL2 (+238201), PARN (+320731); chr16:14727058-147271
08/PARN (−2955), BFAR (+415); chr16:15736811-15736851/KIAA0430 (+192); chr16:16043421-16043536/ABC
C1 (+45); chr16:18495902-18495920/NPIP (−29200), NOMO2 (+77523); chr16:18573469-18573483/NOMO2 (−4
2); chr16:18801430-18801501/RPS15A (+190); chr16:19125225-19125250/ITPRIPL2 (−16); chr16:19144569-19
144698/SYT17 (−35004), ITPRIPL2 (+19380); chr16:20084899-20085303/GPR139 (−1); chr16:20817738-20817
786/LOC81691 (−5), ERI2 (+33); chr16:21170020-21170037/TMEM159 (+117), DNAH3 (+733); chr16:21171001-
21171083/DNAH3 (−280); chr16:21289708-21289749/CRYM (+24675), ANKS4B (+44713); chr16:21295296-21
295340/CRYM (+19086), ANKS4B (+50302); chr16:22201759-22201819/EEF2K (−15803), VWA3A (+97926); ch
r16:2244851 7-22448554/CDR2 (−62598), LOC641298 (−14352); chr16:22825077-22825157/HS3ST2 (−743); chr
16:22825814-22825859/HS3ST2 (−23); chr16:22826351-22826438/HS3ST2 (+535); chr16:23313739-23313779/
SCNN1B (+168); chr16:23568473-23568509/EARS2 (+205); chr16:23706287-23706411/PLK1 (+16148), ERN2
(+18472); chr16:23847054-23847310/PRKCB (−118); chr16:23847680-23847757/PRKCB (+419); chr16:238478
75-23847933/PRKCB (+604); chr16:24266746-24266883/CACNG3 (−59); chr16:24267145-24267220/CACNG3
(+309); chr16:24267753-24267789/CACNG3 (+897); chr16:25704264-25704389/HS3ST4 (+980); chr16:272487
20-27248786/NSMCE1 (+31360), JMJD5 (+33457); chr16:27330114-27330232/IL21R (−108406), IL4R (+4922);
chr16:28073839-28073881/GSG1L (+970); chr16:28074042-28074076/GSG1L (+771); chr16:28074255-28074
417/GSG1L (+494); chr16:28074937-28074955/GSG1L (−116); chr16:28379194-28379218/NPIPL1 (−11231), EI
F3C (+35959); chr16:28385610-28385625/NPIPL1 (−17643), EIF3C (+29547); chr16:28503421-28503478/APO
BR (−2520), CLN3 (+173); chr16:28634328-28634470/NPIPL1 (−21511), SULT1A1 (−13750); chr16:28634894-28
635014/NPIPL1 (−20956), SULT1A1 (−14305); chr16:28834044-28834071/ATXN2L (−356); chr16:28834645-288
34663/ATXN2L (+240); chr16:28874233-28874319/SH2B1 (−802); chr16:28936466-28936529/RABEP2 (+34); c
hr16:28961897-28961970/NFATC2IP (−384); chr16:29086226-29086270/LAT (+90101), NPIPL1 (+329561); chr
16:29093574-29093610/LAT (+97445), NPIPL1 (+322217); chr16:29160249-29160325/LAT (+164140), NPIPL1
(+255522); chr16:29675784-29675990/QPRT (−14554), SPN (+1587); chr16:29816674-29816729/MAZ (−1153);
chr16:29823163-29823214/C16orf53 (−4339), PRRT2 (−220); chr16:29887709-29888476/CDIPT (−13515), SEZ
6L2 (+22492); chr16:30134733-30134772/MAPK3 (−123); chr16:30195526-30195626/CORO1A (+845); chr16:3
0381770-30381809/MYLPF (−4333), TBC1D10B (−268); chr16:30389275-30389336/ZNF48 (−327); chr16:30418
815-30418861/ZNF771 (+103); chr16:30429006-30429060/ZNF771 (+10298), DCTPP1 (+12340); chr16:30485
052-30485158/ITGAL (+1122), ZNF768 (+52805); chr16:30537854-30537878/ZNF768 (+44); chr16:30545993-3
0546036/ZNF747 (+179); chr16:30569336-30569365/ZNF764 (+291); chr16:30572842-30572913/ZNF764 (−323
6); chr16:30621067-30621123/ZNF689 (+587); chr16:30709858-30709893/SRCAP (−586); chr16:30933680-309
33709/FBXL19 (−2201); chr16:30960370-30960419/ORAI3 (−10); chr16:31075680-31075726/ZNF668 (+706); ch
r16:31105659-31105726/VKORC1 (+583); chr16:31191980-31192018/FUS (+568); chr16:31368514-31368596/I
TGAD (−36078), ITGAX (+2046); chr16:31439105-31439159/COX6A2 (+617); chr16:31488049-31488297/SLC5
A2 (−6266), TGFB1I1 (+4697); chr16:31488851-31488906/SLC5A2 (−5560), TGFB1I1 (+5403); chr16:31549095-
31549116/ZNF720 (−175460), AHSP (+9903); chr16:31580164-31580310/ZNF720 (−144329), AHSP (+41034); c
hr16:31830729-31830775/ZNF267 (−54327), ZNF720 (+106186); chr16:31830938-31830978/ZNF267 (−54121),
ZNF720 (+106392); chr16:33612367-33612502/BC068290 (−172076), TP53TG3B (+350315); chr16:33853363-
33853421/BC068290 (+68881); chr16:34489903-34489976/BC068290 (+705429); chr16:46404949-46404995/
SHCBP1 (+250339); chr16:46722678-46722717/ORC6 (−860), VPS35 (+446); chr16:46722844-46722962/ORC
6 (−655), VPS35 (+241); chr16:46865329-46865365/MYLK3 (−83126), GPT2 (−52961); chr16:48089277-480893
31/ABCC12 (+91377), PHKB (+594094); chr16:48643923-48643948/N4BP1 (+184); chr16:48844508-48845119/
N4BP1 (−200694), CBLN1 (+470928); chr16:49311990-49312032/N4BP1 (−667891), CBLN1 (+3731); chr16:49
314822-49314959/CBLN1 (+851); chr16:49316387-49316408/CBLN1 (−656); chr16:49316776-49316844/CBLN
1 (−1068); chr16:49475481-49475626/CBLN1 (−159812), ZNF423 (+385364); chr16:49732566-49732622/CBLN
1 (−416852), ZNF423 (+128324); chr16:49872512-49872806/TMEM188 (−186530), ZNF423 (−11741); chr16:498
89634-49889658/TMEM188 (−169543), ZNF423 (−28728); chr16:49891406-49891452/TMEM188 (−167760), ZN
F423 (−30511); chr16:50187923-50187941/ADCY7 (−133891), PAPD5 (+1103); chr16:50308403-50308533/ADC
Y7 (−13355), PAPD5 (+121639); chr16:50715180-50715218/SNX20 (+65); chr16:50875168-50875194/CYLD
(+99220), SALL1 (+310002); chr16:51168338-51168641/SALL1 (+16693), CYLD (+392529); chr16:51169000-511
69034/SALL1 (+16166), CYLD (+393056); chr16:51183362-51183494/SALL1 (+1755), CYLD (+407467); chr16:
51183720-51183963/SALL1 (+1341), CYLD (+407881); chr16:51184431-51184601/SALL1 (+667); chr16:51184
958-51185054/SALL1 (+177); chr16:51185292-51185458/SALL1 (−192); chr16:51185965-51186025/SALL1 (−81
2); chr16:51187592-51187695/SALL1 (−2461); chr16:51188610-51188696/SALL1 (−3470); chr16:51189208-511
89282/SALL1 (−4062); chr16:51190038-51190121/SALL1 (−4897); chr16:51219359-51219444/SALL1 (−34219);
chr16:52581505-52581537/TOX3 (−715); chr16:53165591-53165634/RBL2 (−302738), CHD9 (+76668); chr16:5
4315986-54316061/IRX3 (+4354), FTO (+578149); chr16:54317947-54317988/IRX3 (+2410), FTO (+580093); c
hr16:54318589-54318621/IRX3 (+1773), FTO (+580730); chr16:54318765-54318853/IRX3 (+1569), FTO (+580
934); chr16:54321819-54321907/IRX3 (−1485); chr16:54322298-54322447/IRX3 (−1995); chr16:54322937-5432
3012/IRX3 (−2597); chr16:54323354-54323509/IRX3 (−3054); chr16:54324293-54324442/IRX3 (−3990); chr16:5
4324593-54324636/IRX3 (−4237); chr16:54611696-54611724/IRX5 (−353401), IRX3 (−291332); chr16:54966169-
54966659/IRX6 (−392057), IRX5 (+1303); chr16:54967265-54967414/IRX6 (−391131), IRX5 (+2229); chr16:549
67667-54967702/IRX6 (−390786), IRX5 (+2574); chr16:54969399-54969548/IRX6 (−388997), IRX5 (+4363); chr
16:54970258-54970357/IRX6 (−388163), IRX5 (+5197); chr16:54971058-54971116/IRX6 (−387384), IRX5 (+597
6); chr16:54971566-54971721/IRX6 (−386827), IRX5 (+6533); chr16:54972300-54972349/IRX6 (−386146), IRX5
(+7214); chr16:55357573-55357611/IRX6 (−879); chr16:55357941-55357991/IRX6 (−505); chr16:55358416-553
58565/IRX6 (+20); chr16:55358676-55358784/IRX6 (+259); chr16:55361546-55361674/MMP2 (−151471), IRX6
(+3139); chr16:55362773-55362962/MMP2 (−150213), IRX6 (+4397); chr16:55365219-55365434/MMP2 (−1477
54), IRX6 (+6856); chr16:55512763-55512935/MMP2 (−232); chr16:55513364-55513482/MMP2 (+342); chr16:5
5689778-55689852/SLC6A2 (−741); chr16:55690380-55690453/SLC6A2 (−139); chr16:55690577-55690761/SL
C6A2 (+113); chr16:56224833-56224880/GNAO1 (−394); chr16:56228426-56228515/GNAO1 (+3220), AMFR
(+230973); chr16:56651124-56651238/MT1E (−8404), MT2A (+8703); chr16:56669270-56669605/MT1A (−3140);
chr16:56672173-56672221/MT1A (−381); chr16:56672386-56672513/MT1A (−128); chr16:56681916-56681953/
MT1B (−3876); chr16:56691837-56691884/MT1F (+6); chr16:56696747-56696833/MT1F (+4935), MT1G (+5187);
chr16:56703399-56703438/MT1G (−1442); chr16:56703560-56703621/MT1G (−1614); chr16:56709893-56709
949/MT1IP (+272); chr16:56764407-56764444/NUP93 (+409); chr16:56966468-56966496/HERPUD1 (+734); c
hr16:57278806-57278842/ARL2BP (−214); chr16:57318708-57318783/PLLP (−162); chr16:57480752-57480792/
COQ9 (−565), CIAPIN1 (+597); chr16:57509861-57509966/DOK4 (+10471), POLR2C (+13363); chr16:5766348
0-57663574/GPR97 (−38630), GPR56 (+9617); chr16:57836101-57836154/KIFC3 (+311); chr16:58060578-580
60638/MMP15 (+1326), C16orf80 (+102688); chr16:58129262-58129359/C16orf80 (+33985), MMP15 (+70029);
chr16:58231265-58231297/CSNK2A2 (+501); chr16:58497033-58497060/NDRG4 (−502); chr16:58497336-584
97469/NDRG4 (−146); chr16:58550114-58550163/SETD6 (+756); chr16:58663370-58663426/CNOT1 (+352); c
hr16:62069084-62069136/CDH8 (+1629); chr16:62069274-62069336/CDH8 (+1434); chr16:62070113-6207024
5/CDH8 (+560); chr16:63651555-63651574/NONE; chr16:65156895-65156939/CDH11 (−998); chr16:65157838-
65157863/CDH11 (−1932); chr16:66461384-66461511/BEAN1 (+248); chr16:66462133-66462187/BEAN1 (+96
0); chr16:66613126-66613221/CMTM2 (−177); chr16:66638222-66638268/CMTM3 (+17); chr16:66967866-6696
7928/CES2 (−450), FAM96B (+423); chr16:66982614-66982672/CES3 (−12489), CES2 (+14296); chr16:669829
38-66983018/CES3 (−12154), CES2 (+14631); chr16:67184057-67184120/B3GNT9 (+813), BC007896 (+970);
chr16:67199591-67199959/NOL3 (−7990), HSF4 (+2487); chr16:67218695-67218723/EXOC3L1 (+5398), NOL3
(+10944); chr16:67428252-67428323/TPPP3 (−867); chr16:67428539-67428701/TPPP3 (−1199); chr16:674289
11-67429022/TPPP3 (−1546); chr16:67450547-67450605/ZDHHC1 (−237); chr16:67464824-67464885/HSD11B
2 (−181); chr16:67555194-67555233/AGRP (−37498), FAM65A (−8326); chr16:67562690-67562705/FAM65A (−8
42); chr16:67564267-67564312/FAM65A (+750); chr16:67596724-67596765/CTCF (+435); chr16:67693788-67
693831/PARD6A (−1041), ACD (+908); chr16:67753011-67753048/GFOD2 (+243); chr16:67840677-67840711/
TSNAXIP1 (−316), RANBP10 (−139); chr16:67867576-67867632/THAP11 (−8609), TSNAXIP1 (+26594); chr16:6
7927573-67927637/PSKH1 (+430); chr16:68057773-68057842/DDX28 (−38), DUS2L (+604); chr16:68118645-6
8118725/NFATC3 (−584); chr16:68269885-68269896/ESRP2 (+245); chr16:68270008-68270022/ESRP2 (+121);
chr16:68390172-68390251/PRMT7 (+45335), SMPD3 (+92197); chr16:68481817-68481955/SMPD3 (+523); c
hr16:68676605-68676841/CDH3 (−1428); chr16:68679344-68679362/CDH1 (−91842), CDH3 (+1202); chr16:691
41662-69141722/HAS3 (+2225), CHTF8 (+24379); chr16:69166371-69166402/CHTF8 (−316), CIRH1A (−112), C
HTF8 (+106); chr16:69363753-69363787/PDF (+728); chr16:69788907-69788961/NOB1 (−105); chr16:7041581
8-70415853/ST3GAL2 (+57155), DDX19B (+82764); chr16:70731068-70731182/MTSS1L (−11171), VAC14 (+1
03936); chr16:70771496-70771548/MTSS1L (−51568), VAC14 (+63539); chr16:71459745-71459956/ZNF23 (+3
6266), CALB2 (+67235); chr16:71460089-71460270/ZNF23 (+35937), CALB2 (+67564); chr16:71475549-7147
5587/ZNF23 (+20549), CALB2 (+82952); chr16:71518306-7158342/ZNF23 (−22207), ZNF19 (+4930); chr16:71
757620-71757692/PHLPP2 (−8952), AP1G1 (+85320); chr16:71918277-71918302/ZNF821 (−197); chr16:71918
445-71918540/ZNF821 (−400); chr16:72698163-72698208/PMFBP1 (−492122), ZFHX3 (+384088); chr16:72698
763-72698838/PMFBP1 (−492737), ZFHX3 (+383473); chr16:72821149-72821299/PMFBP1 (−615160), ZFHX3
(+261050); chr16:73091885-73091900/ZFHX3 (−9619); chr16:73097300-73097376/ZFHX3 (−15064); chr16:7309
9020-73099107/ZFHX3 (−16790); chr16:73099971-73100271/ZFHX3 (−17847); chr16:73100402-73100475/ZFH
X3 (−18165); chr16:73517084-73517220/PSMD7 (−813521), ZFHX3 (−434878); chr16:74440563-74440617/CLE
C18B (+14778), NPIPL2 (+28719); chr16:74440806-74440820/CLEC18B (+14555), NPIPL2 (+28942); chr16:75
033530-75033560/ZNRF1 (+630); chr16:75284183-75284222/CTRB2 (−43131), BCAR1 (+15702); chr16:75284
546-75284629/CTRB2 (−43516), BCAR1 (+15317); chr16:75284869-75284892/CTRB2 (−43809), BCAR1 (+1502
4); chr16:75285389-75285434/CTRB2 (−44340), BCAR1 (+14493); chr16:75528869-75528922/CHST6 (+30); ch
r16:75600154-75600172/GABARAPL2 (−86); chr16:77468681-77468738/ADAMTS18 (+301); chr16:77468860-7
7468969/ADAMTS18 (+96); chr16:77821872-77821903/VAT1L (−595); chr16:79200997-79201089/MAF (+4335
79); chr16:79425535-79425559/MAF (+209075); chr16:79623617-79623797/MAF (+10915); chr16:79632817-7
9633073/MAF (+1677); chr16:79633577-79633613/MAF (+1027); chr16:79804558-79804596/DYNLRB2 (−7702
77), MAF (−169955); chr16:80837354-80837395/CDYL2 (+800); chr16:81069601-81069622/ATMIN (+154); chr1
6:81110503-81110546/GCSH (+19455), ATMIN (+41067); chr16:81348308-81348354/GAN (−240); chr16:82203
449-82203521/MPHOSPH6 (+344); chr16:82661412-82661468/CDH13 (+1041); chr16:84002792-84002810/NE
CAB2 (+564); chr16:84029506-84029596/NECAB2 (+27314), SLC38A8 (+46211); chr16:84262294-84262348/K
CNG4 (+11035), ADAD2 (+37598); chr16:85116423-85116476/KIAA0182 (−530474), KIAA0513 (+55040); chr16:
85138764-85138903/KIAA0182 (−508090), KIAA0513 (+77424); chr16:85171053-85171121/KIAA0182 (−47583
7), KIAA0513 (+109677); chr16:85286283-85286371/KIAA0182 (−360597), KIAA0513 (+224917); chr16:852926
89-85292791/KIAA0182 (−354184), KIAA0513 (+231330); chr16:85337153-85337243/KIAA0182 (−309726), KIA
A0513 (+275788); chr16:85343228-85343355/KIAA0182 (−303632), KIAA0513 (+281882); chr16:85344435-853
44570/KIAA0182 (−302421), KIAA0513 (+283093); chr16:85427513-85427587/KIAA0182 (−219374), KIAA0513
(+366140); chr16:85436749-85436840/KIAA0182 (−210129), KIAA0513 (+375385); chr16:85482379-85482436/K
IAA0182 (−164516), KIAA0513 (+420998); chr16:85600958-85601085/KIAA0182 (−45902), KIAA0513 (+539612);
chr16:85648095-85648236/KIAA0182 (+1242), GINS2 (+74422); chr16:85648543-85648641/KIAA0182 (+166
8), GINS2 (+73996); chr16:85648893-85648998/KIAA0182 (+2022), GINS2 (+73642); chr16:85783751-857838
23/GINS2 (−61199), COX4NB (+49361); chr16:85792866-85793032/GINS2 (−70361), COX4NB (+40199); chr16:
86103612-86103759/FOXF1 (−440447), IRF8 (+170912); chr16:86439293-86439368/FOXF1 (−104802), IRF8
(+506557); chr16:86528384-86528735/FOXF1 (−15573), IRF8 (+595786); chr16:86529610-86529704/FOXF1 (−14
476), IRF8 (+596883); chr16:86530445-86530505/FOXF1 (−13658), IRF8 (+597701); chr16:86530805-8653082
0/FOXF1 (−13320), IRF8 (+598039); chr16:86530993-86531016/FOXF1 (−13128), IRF8 (+598231); chr16:86531
289-86531374/FOXF1 (−12801), IRF8 (+598558); chr16:86531481-86531527/FOXF1 (−12629), IRF8 (+598730);
chr16:86531656-86531701/FOXF1 (−12454), IRF8 (+598905); chr16:86531990-86532038/FOXF1 (−12119), IR
F8 (+599240); chr16:86532213-86532408/FOXF1 (−11822), IRF8 (+599537); chr16:86535992-86536131/FOXF
1 (−8071), IRF8 (+603288); chr16:86538778-86538820/FOXF1 (−5334), IRF8 (+606025); chr16:86539427-86539
463/FOXF1 (−4688); chr16:86542239-86542273/FOXF1 (−1877); chr16:86544558-86544734/FOXF1 (+513); chr
16:86544858-86545059/FOXF1 (+826); chr16:86546627-86546697/FOXF1 (+2529), MTHFSD (+42179); chr16:
86548828-86548905/FOXF1 (+4734), MTHFSD (+39974); chr16:86549125-86549135/FOXF1 (+4997), MTHFS
D (+39711); chr16:86549275-86549345/FOXF1 (+5177), MTHFSD (+39531); chr16:86549556-86549624/FOXF
1 (+5457), MTHFSD (+39251); chr16:86550291-86550488/FOXF1 (+6257), MTHFSD (+38451); chr16:8659938
5-86599480/FOXC2 (−1424); chr16:86601817-86602037/FOXL1 (−10188), FOXC2 (+1070); chr16:86612283-86
612455/FOXL1 (+254); chr16:86612596-86612664/FOXL1 (+515); chr16:86612800-86613152/FOXL1 (+861); c
hr16:86766748-86766775/FOXL1 (+154647), FBXO31 (+650632); chr16:86962681-86962712/FOXL1 (+350582),
FBXO31 (+454697); chr16:86962860-86963004/FOXL1 (+350817), FBXO31 (+454462); chr16:86968306-869
68372/FOXL1 (+356224), FBXO31 (+449055); chr16:86990271-86990363/FOXL1 (+378202), FBXO31 (+42707
7); chr16:87050635-87050759/FBXO31 (+366697), FOXL1 (+438582); chr16:87086848-87087028/FBXO31 (+3
30456), FOXL1 (+474823); chr16:87270133-87270247/FBXO31 (+147204), FOXL1 (+658075); chr16:87416712-
87416735/FBXO31 (+670); chr16:87635971-87636008/JPH3 (−509); chr16:87855415-87855489/SLC7A5 (+47
648), JPH3 (+218953); chr16:87902805-87902820/SLC7A5 (+287); chr16:87993363-87993423/ZNF469 (−5004
86), BANP (+8355); chr16:88367324-88367364/ZNF469 (−126535), BANP (+382306); chr16:88496611-8849679
0/ZFPM1 (−23313), ZNF469 (+2822); chr16:88497136-88497180/ZFPM1 (−22856), ZNF469 (+3279); chr16:885
18785-88518836/ZFPM1 (−1203); chr16:88589780-88589844/ZC3H18 (−46977), ZFPM1 (+69798); chr16:88600
503-88600511/ZC3H18 (−36282), ZFPM1 (+80493); chr16:88600797-88600888/ZC3H18 (−35946), ZFPM1 (+80
829); chr16:88616918-88617012/ZC3H18 (−19824), ZFPM1 (+96951); chr16:88700512-88700602/IL17C (−4444);
chr16:88701003-88701343/IL17C (−3828); chr16:88752975-88753000/SNAI3 (−106); chr16:88758478-887586
17/SNAI3 (−5666), RNF166 (+14281); chr16:88767227-88767316/SNAI3 (−14390), RNF166 (+5557); chr16:887
69900-88769941/CTU2 (−2970), RNF166 (+2908); chr16:88812307-88812357/PIEZO1 (+39040), CTU2 (+3944
1); chr16:88844844-88844922/PIEZO1 (+6489), CTU2 (+71992); chr16:88851294-88851348/PIEZO1 (+51); chr
16:88858514-88858660/CDT1 (−11599), PIEZO1 (−7215); chr16:88866383-88866456/CDT1 (−3766); chr16:8886
8608-88868734/CDT1 (−1515); chr16:88870300-88870350/CDT1 (+139); chr16:89003086-89003234/PABPN1L
(−70092), CBFA2T3 (+40344); chr16:89033863-89034075/PABPN1L (−100901), CBFA2T3 (+9535); chr16:8904
1912-89041964/PABPN1L (−108870), CBFA2T3 (+1566); chr16:89042315-89042383/PABPN1L (−109281), CBF
A2T3 (+1155); chr16:89043373-89043441/CBFA2T3 (+97); chr16:89044515-89044613/CBFA2T3 (−1060); chr1
6:89044779-89045001/CBFA2T3 (−1386); chr16:89261143-89261212/SLC22A31 (+5351), CDH15 (+23015); ch
r16:89267825-89267957/SLC22A31 (−1362); chr16:89268992-89269013/SLC22A31 (−2474); chr16:89573472-8
9573585/SPG7 (−1276); chr16:89641355-89641413/CPNE7 (−792); chr16:89642529-89642560/CPNE7 (+369);
chr16:89642797-89642823/CPNE7 (+634); chr16:89642929-89643017/CPNE7 (+797); chr16:89767931-897679
57/CDK10 (+14868), C16orf7 (+19450); chr16:89768197-89768247/CDK10 (+15146), C16orf7 (+19172); chr16:
89787087-89787112/ZNF276 (−852), C16orf7 (+294); chr16:89789245-89789327/C16orf7 (−1892), ZNF276 (+1
334); chr16:89940943-89941004/TCF25 (+980); chr16:90038552-90038590/CENPBD1 (+669); chr16:90039469-
90039503/AFG3L1P (−4600), CENPBD1 (−246); chr16:90075578-90075667/DBNDD1 (+906); chr16:90086047-
90086126/GAS8 (−2921); chr16:90143643-90143696/PRDM7 (−1332); chr17:29890-29994/DOC2B (+1478); chr
17:234746-234795/RPH3AL (−32138), VPS53 (+383325); chr17:260456-260521/RPH3AL (−57856), VPS53 (+35
7607); chr17:617958-617979/VPS53 (+127); chr17:635936-635959/FAM57A (+101); chr17:655517-655539/GE
MIN4 (−27); chr17:655770-655880/GEMIN4 (−324); chr17:882873-882912/NXN (+105); chr17:883526-883582/N
XN (−556); chr17:943269-943307/TIMM22 (+42931), ABR (+139843); chr17:943548-943617/TIMM22 (+43226),
ABR (+139548); chr17:1012417-1012469/ABR (+70688), TIMM22 (+112086); chr17:1083469-1083490/ABR (−3
49); chr17:1107066-1107143/BHLHA9 (−66753), ABR (−23974); chr17:1131336-1131423/ABR (−48249), BHLHA
9 (−42478); chr17:1132838-1132870/ABR (−49723), BHLHA9 (−41004); chr17:1133673-1133707/ABR (−50559),
BHLHA9 (−40168); chr17:1160228-1160270/ABR (−77118), BHLHA9 (−13609); chr17:1173888-1174238/BHLHA
9 (+205); chr17:1174362-1174384/BHLHA9 (+515); chr17:1320785-1320857/YWHAE (−17265), CRK (+38740);
chr17:1394663-1394817/CRK (−35179), MYO1C (+1261); chr17:1465561-1465586/PITPNA (+536); chr17:1532
765-1532798/SLC43A2 (−652); chr17:1552698-1552881/SCARF1 (−3749), RILP (+602); chr17:1588019-158804
2/PRPF8 (+145); chr17:1901789-1901813/RTN4RL1 (+26377), RPA1 (+168528); chr17:1956749-1956782/HIC
1 (−2838); chr17:1960007-1960123/HIC1 (+461); chr17:1960235-1960585/HIC1 (+806); chr17:1960748-196085
9/HIC1 (+1200), SMG6 (+246265); chr17:1960965-1961018/HIC1 (+1388), SMG6 (+246077); chr17:1962099-1
962301/HIC1 (+2596), SMG6 (+244869); chr17:2297309-2297378/MNT (+6914), SGSM2 (+56538); chr17:2305
636-2305725/MNT (−1423); chr17:2414944-2414999/METTL16 (+228); chr17:2615137-2615185/KIAA0664 (−23
4); chr17:2696047-2696094/RAP1GAP2 (−3661); chr17:2700621-2700780/RAP1GAP2 (+969); chr17:2756166-
2756268/RAP1GAP2 (+56485), OR1D5 (+210684); chr17:2838933-2838975/OR1D5 (+127947), RAP1GAP2
(+139222); chr17:2839143-2839192/OR1D5 (+127733), RAP1GAP2 (+139436); chr17:3432284-3432404/TRPV3
(+28945), ASPA (+53048); chr17:3438938-3439029/TRPV3 (+22305), ASPA (+59688); chr17:3571977-357200
1/TMEM93 (−101), TAX1BP3 (−16); chr17:3572709-3572751/TAX1BP3 (−757), TMEM93 (+640); chr17:3599173-
3599244/P2RX5 (+489); chr17:3795639-3795684/CAMKK1 (−1625); chr17:3814784-3814830/CAMKK1 (−20770),
P2RX1 (+5153); chr17:3819195-3819242/P2RX1 (+741); chr17:3819373-3819418/P2RX1 (+564); chr17:3908
130-3908185/ATP2A3 (−40400), ZZEF1 (+138095); chr17:4607042-4607098/PELP1 (+562); chr17:4641833-464
1953/ZMYND15 (−1426), CXCL16 (+1330); chr17:4642311-4642483/ZMYND15 (−922), CXCL16 (+826); chr17:4
651566-4651662/TM4SF5 (−23573), ZMYND15 (+8295); chr17:4693074-4693142/VMO1 (−3379), BC150535
(+520), GLTPD2 (+854); chr17:4803687-4803928/CHRNE (+2561), MINK1 (+67173); chr17:4804078-4804151/C
HRNE (+2254), MINK1 (+67480); chr17:4804291-4804398/CHRNE (+2024), MINK1 (+67710); chr17:4843752-4
843820/SLC25A11 (−324), RNF167 (+156); chr17:4981822-4981951/ZFP3 (+133); chr17:5000936-5000963/ZF
P3 (+19196), ZNF232 (+25447); chr17:5014939-5014988/ZNF232 (+11433), ZFP3 (+33210); chr17:5015130-50
15185/ZNF232 (+11239), ZFP3 (+33404); chr17:5019663-5019737/ZNF232 (+6697), ZFP3 (+37946); chr17:502
6108-5026146/ZNF232 (+270); chr17:5323389-5323426/NUP88 (−349), RPAIN (+447); chr17:5342594-5342656/
C1QBP (−154); chr17:5404091-5404222/MIS12 (+13910), NLRP1 (+83675); chr17:5487163-5487267/NLRP1
(+617); chr17:5783108-5783181/NLRP1 (−295313), WSCD1 (−190789); chr17:5800913-5801043/NLRP1 (−31314
6), WSCD1 (−172956); chr17:5974246-5974297/WSCD1 (+338); chr17:6459775-6459791/PITPNM3 (+94); chr1
7:6543203-6543260/TXNDC17 (−990); chr17:6543466-6543492/TXNDC17 (−743), KIAA0753 (+768); chr17:654
3665-6543736/TXNDC17 (−521), KIAA0753 (+546); chr17:6555437-6555465/MED31 (−497); chr17:6617174-661
7299/SLC13A5 (−497); chr17:6899127-6899161/ALOX12 (−240); chr17:6918146-6918167/C17orf49 (+693); chr
17:6925693-6925723/BCL6B (−661); chr17:6926155-6926198/BCL6B (−192); chr17:6939243-6939269/SLC16A
13 (−138); chr17:6945623-6945647/SLC16A11 (+1607), SLC16A13 (+6241); chr17:6946153-6946175/SLC16A1
1 (+1078), SLC16A13 (+6770); chr17:6946412-6946476/SLC16A11 (+798); chr17:7077705-7077743/ASGR2
(−59594), ASGR1 (+5159); chr17:7080953-7080980/ASGR2 (−62837), ASGR1 (+1916); chr17:7081187-7081226/
ASGR2 (−63077), ASGR1 (+1676); chr17:7138009-7138077/DVL2 (−180); chr17:7146515-7146549/PHF23 (−37
07), GABARAP (−779); chr17:7165130-7165155/CLDN7 (+1121), C17orf81 (+9592); chr17:7165468-7165695/C
LDN7 (+682); chr17:7166306-7166376/CLDN7 (−77); chr17:7197555-7197579/YBX2 (+309); chr17:7240012-72
40098/ACAP1 (+207); chr17:7240479-7240548/ACAP1 (+666); chr17:7287256-7287270/TNK1 (+2970), PLSCR
3 (+10899); chr17:7287422-7287591/TNK1 (+3214), PLSCR3 (+10655); chr17:7308851-7308875/NLGN2 (−263
9), C17orf61 (−1413); chr17:7339523-7339568/FGF11 (−3143), TMEM102 (+784); chr17:7341503-7341649/FGF
11 (−1113); chr17:7341756-7341822/FGF11 (−900); chr17:7350323-7350400/SLC35G6 (−34359), CHRNB1 (+19
56); chr17:7387531-7387572/POLR2A (−146), ZBTB4 (+16); chr17:7464690-7464723/SENP3 (−602); chr17:760
8421-7608435/EFNB3 (−92); chr17:7608576-7608613/EFNB3 (+75); chr17:7620295-7620334/DNAH2 (−2724); c
hr17:7759289-7759320/CYB5D1 (−1759), TMEM88 (+921); chr17:7761162-7761197/LSMD1 (−8), CYB5D1 (+11
6); chr17:7919772-7919806/ALOX15B (−22569), GUCY2D (+13801); chr17:7942673-7942759/ALOX15B (+358);
chr17:7975744-7975774/ALOX12B (+15262), ALOX15B (+33401); chr17:8024155-8024224/ALOXE3 (−1956);
chr17:8054550-8054696/HES7 (−27213), PER1 (+1130); chr17:8066433-8066510/VAMP2 (−179); chr17:806661
8-8066691/VAMP2 (−362); chr17:8077105-8077137/VAMP2 (−10828), TMEM107 (+2593); chr17:8113719-8113
757/AURKB (+145); chr17:8126467-8126524/AURKB (−12613), CTC1 (+24917); chr17:8150444-8150617/PFAS
(−2065), CTC1 (+882); chr17:8286405-8286496/RPL26 (+114); chr17:8339889-8339943/NDEL1 (+737); chr17:
8868668-8868814/NTN1 (−56118), PIK3R5 (−52907); chr17:8905284-8905384/PIK3R5 (−89500), NTN1 (−19525);
chr17:8905692-8905738/PIK3R5 (−89881), NTN1 (−19144); chr17:8907728-8907776/PIK3R5 (−91918), NTN1
(−17107); chr17:8926539-8926561/NTN1 (+1691), STX8 (+552725); chr17:8926674-8926703/NTN1 (+1830), ST
X8 (+552586); chr17:8926866-8926902/NTN1 (+2025), STX8 (+552391); chr17:9135925-9136059/NTN1 (+211
133), STX8 (+343283); chr17:9143296-9143376/NTN1 (+218477), STX8 (+335939); chr17:9143537-9143607/N
TN1 (+218713), STX8 (+335703); chr17:9143790-9143835/NTN1 (+218954), STX8 (+335462); chr17:9479147-
9479185/WDR16 (−778), STX8 (+109); chr17:10101110-10101131/GAS7 (+747); chr17:11145314-11145389/S
HISA6 (+612); chr17:11924527-11924610/MAP2K4 (+434); chr17:12325239-12325342/MYOCD (−243916), MA
P2K4 (+401156); chr17:12877138-12877185/ELAC2 (+44219), ARHGAP44 (+184333); chr17:12877320-12877
536/ELAC2 (+43953), ARHGAP44 (+184599); chr17:13503945-13504194/ELAC2 (−582689), HS3ST3A1 (+117
4); chr17:13504959-13505001/HS3ST3A1 (+264); chr17:14200907-14200930/HS3ST3B1 (−3587); chr17:14212
521-14212551/HS3ST3B1 (+8030), PMP22 (+951557); chr17:15244852-15244987/TEKT3 (+38); chr17:158207
46-15820777/ADORA2B (−27469), MEIS3P1 (+130598); chr17:15821184-15821210/ADORA2B (−27034), MEIS
3P1 (+131033); chr17:15847757-15847779/ADORA2B (−463); chr17:16283927-16283956/UBB (−425); chr17:16
395319-16395341/C17orf76-AS1 (+53029), ZNF287 (+77190); chr17:16570118-16570208/ZNF624 (−12996), K
RT16P2 (+165984); chr17:16570319-16570360/ZNF624 (−13173), KRT16P2 (+165807); chr17:16777281-16777
334/KRT16P2 (−41161), TNFRSF13B (+98094); chr17:16945967-16945999/MPRIP (−121991), TNFRSF13B (−7
0581); chr17:17170247-17170300/FLCN (−29772), COPS3 (+14343); chr17:17287225-17287254/MED9 (−93060),
NT5M (+80560); chr17:17295540-17295618/MED9 (−84721), NT5M (+88899); chr17:17380175-17380216/ME
D9 (−104); chr17:17586304-17586321/RAI1 (+1526), SREBF1 (+154012); chr17:17627993-17628109/RAI1 (+43
264), SREBF1 (+112274); chr17:17628219-17628267/RAI1 (+43456), SREBF1 (+112082); chr17:17661380-17
661447/RAI1 (+76627), SREBF1 (+78911); chr17:17696385-17696447/SREBF1 (+43909), RAI1 (+111629); chr
17:17942433-17942477/ATPAF2 (+25); chr17:17943318-17943340/ATPAF2 (−849); chr17:18086258-18086312/
ALKBH5 (−582); chr17:18129033-18129064/LLGL1 (+113); chr17:18163909-18163935/FLII (−1867), SMCR7
(−502); chr17:18528494-18528534/TBC1D28 (+19226), LGALS9C (+148415); chr17:18538360-18538454/TBC1D
28 (+9333), LGALS9C (+158308); chr17:19318728-19318847/SLC47A1 (−118379), RNF112 (+4297); chr17:195
51783-19551819/ALDH3A2 (−263); chr17:20747026-20747060/USP22 (+199309), CDRT15L2 (+264006); chr17:
20747179-20747201/USP22 (+199162), CDRT15L2 (+264153); chr17:20805955-20806021/USP22 (+140364),
CDRT15L2 (+322951); chr17:20944215-20944282/USP22 (+2103), CDRT15L2 (+461212); chr17:21002662-21
002711/USP22 (−56335), DHRS7B (−27554); chr17:21156941-21156994/TMEM11 (−39060), MAP2K3 (−31000);
chr17:21367075-21367188/MTRNR2L1 (−655305), KCNJ18 (+58684); chr17:21367373-21367445/MTRNR2L1
(−655028), KCNJ18 (+58961); chr17:21414858-21414967/MTRNR2L1 (−607524), KCNJ18 (+106465); chr17:214
15453-21415476/MTRNR2L1 (−606972), KCNJ18 (+107017); chr17:21455042-21455071/MTRNR2L1 (−567380),
KCNJ18 (+146609); chr17:25676094-25676480/KSR1 (−122749), WSB1 (+55181); chr17:25680982-25681023/
KSR1 (−118033), WSB1 (+59897); chr17:25793823-25793905/KSR1 (−5172), WSB1 (+172758); chr17:2612085
1-26120901/NOS2 (+6679), LGALS9 (+162702); chr17:26220327-26220401/NLK (−149324), NOS2 (−92809); ch
r17:26368591-26368637/NLK (−1074); chr17:26554297-26554487/KRT18P55 (+80016), NLK (+184704); chr17:
26554753-26554791/KRT18P55 (+79636), NLK (+185084); chr17:26634268-26634287/KRT18P55 (+130); chr1
7:26662300-26662339/TNFAIP1 (−228), IFT20 (+175); chr17:26684172-26684228/TMEM199 (−487), POLDIP2
(+403); chr17:26699303-26699404/VTN (−1981), SARM1 (+367); chr17:26711815-26711874/SARM1 (+12858),
SLC46A1 (+21385); chr17:26879358-26879389/UNC119 (+272); chr17:26903871-26903937/ALDOC (+47); chr
17:26926026-26926083/SPAG5 (+1); chr17:26988721-26988761/SUPT6H (−561), SDF2 (+449); chr17:2703878
5-27038892/PROCA1 (+33); chr17:27044220-27044250/RPL23A (−2765), RAB34 (+1051); chr17:27044419-270
44581/RPL23A (−2500), RAB34 (+786); chr17:27044744-27044880/RPL23A (−2188), RAB34 (+474); chr17:270
47400-27047431/RAB34 (−2130), RPL23A (+416); chr17:27053584-27053634/NEK8 (−2223), TLCD1 (−379); chr
17:27054041-27054063/NEK8 (−1780), TLCD1 (−822); chr17:27071281-27071309/TRAF4 (+272); chr17:272768
74-27276903/DHRS13 (−46800), PHF12 (+1619); chr17:27503598-27503625/TIAF1 (−100985), MYO18A (+379
5); chr17:27893195-27893344/TP53I13 (−2469), ABHD15 (+772); chr17:27918568-27918605/GIT1 (−1977); chr
17:27940476-27940590/GIT1 (−23923), CORO6 (+7908); chr17:27943723-27943891/GIT1 (−27197), CORO6
(+4634); chr17:27950128-27950259/CORO6 (−1753); chr17:28087998-28088056/CORO6 (−139586), SSH2 (+168
991); chr17:28565435-28565637/SLC6A4 (−2582); chr17:28927103-28927146/LRRC37BP1 (−8323), TBC1D29
(+41083); chr17:28927613-28927624/LRRC37BP1 (−7829), TBC1D29 (+41577); chr17:29249287-29249457/AD
AP2 (+618); chr17:29249961-29250019/RNF135 (−47966), ADAP2 (+1236); chr17:29335759-29335853/NF1 (−8
6139), RNF135 (+37850); chr17:29336075-29336123/NF1 (−85846), RNF135 (+38143); chr17:29421883-29421
899/NF1 (−54); chr17:29719247-29719289/RAB11FIP4 (+626); chr17:29908330-29908396/RAB11FIP4 (+18972
1), C17orf79 (+277963); chr17:30186463-30186515/C17orf79 (−163); chr17:30244357-30244382/SUZ12 (−1967
4), UTP6 (−15641); chr17:30334007-30334083/LRRC37B (−14110), SUZ12 (+70001); chr17:30669062-3066908
2/C17orf75 (+156); chr17:32482483-32482665/ACCN1 (+1251); chr17:32906556-32906598/TMEM132E (−1191);
chr17:32907805-32907873/TMEM132E (+71); chr17:32908147-32908170/TMEM132E (+391); chr17:3295321
9-32953234/TMEM132E (+45459), CCT6B (+335301); chr17:32953728-32953966/TMEM132E (+46079), CCT6
B (+334681); chr17:32964650-32964755/TMEM132E (+56935), CCT6B (+323825); chr17:33446674-33446704/
RFFL (−55930), NLE1 (+22633); chr17:33641110-33641148/SLFN11 (+59591), SLFN5 (+71043); chr17:336727
04-33672794/SLFN11 (+27971), SLFN5 (+102663); chr17:33776355-33776394/SLFN13 (−519); chr17:3413656
5-34136717/TAF15 (+153); chr17:34842884-34842918/ZNHIT3 (+428); chr17:34900456-34900490/GGNBP2
(−264); chr17:34997819-34998007/LHX1 (−296859), MRM1 (+39888); chr17:34998131-34998163/LHX1 (−296625),
MRM1 (+40122); chr17:35291899-35291920/LHX1 (−2862); chr17:35299511-35299546/AATF (−6646), LHX1
(+4757); chr17:35300073-35300150/AATF (−6063), LHX1 (+5340); chr17:35300713-35300812/AATF (−5412), LH
X1 (+5991); chr17:35306151-35306167/AATF (−16); chr17:35766530-35766573/TADA2A (−425), ACACA (+350);
chr17:36104035-36104217/HNF1B (+970); chr17:36104551-36104643/HNF1B (+499); chr17:36105350-36105
458/HNF1B (−308); chr17:36582015-36582204/ARHGAP23 (−2610); chr17:36610236-36610293/ARHGAP23
(+25545), SRCIN1 (+151918); chr17:36666142-36666336/ARHGAP23 (+81519), SRCIN1 (+95944); chr17:36666
447-36666692/ARHGAP23 (+81850), SRCIN1 (+95613); chr17:36666831-36666991/ARHGAP23 (+82191), SR
CIN1 (+95272); chr17:36714490-36714528/SRCIN1 (+47674), ARHGAP23 (+129789); chr17:36717793-367179
25/SRCIN1 (+44324), ARHGAP23 (+133139); chr17:36718096-36718255/SRCIN1 (+44007), ARHGAP23 (+133
456); chr17:36718495-36718567/SRCIN1 (+43652), ARHGAP23 (+133811); chr17:36719377-36719422/SRCIN
1 (+42783), ARHGAP23 (+134680); chr17:36734989-36735009/SRCIN1 (+27184), ARHGAP23 (+150279); chr1
7:36762342-36762362/SRCIN1 (−169); chr17:36829692-36829779/SRCIN1 (−67553), MLLT6 (−32137); chr17:36
862067-36862089/MLLT6 (+205); chr17:36981169-36981216/PIP4K2B (−25035), RPL23 (+28860); chr17:37009
590-37009648/RPL23 (+434); chr17:37307514-37307531/PLXDC1 (+379); chr17:37308034-37308156/PLXDC1
(−193); chr17:37321625-37321787/ARL5C (+708); chr17:37381430-37381570/STAC2 (+540); chr17:37381727-
37381825/STAC2 (+264); chr17:37557180-37557248/FBXL20 (+695); chr17:37558000-37558008/FBXL20 (−95);
chr17:37607607-37607662/MED1 (−108); chr17:37729643-37729715/NEUROD2 (+34496), CDK12 (+111940);
chr17:37729909-37729927/NEUROD2 (+34257), CDK12 (+112179); chr17:37752939-37753021/NEUROD2
(+11195), CDK12 (+135241); chr17:37764138-37764263/NEUROD2 (−26); chr17:37894915-37894953/GRB7 (+7
72); chr17:37895958-37896009/GRB7 (+1822), IKZF3 (+124457); chr17:38024330-38024423/IKZF3 (−3936), Z
PBP2 (−78); chr17:38255763-38255830/NR1D1 (+1176), THRA (+36729); chr17:38334445-38334528/RAPGEF
L1 (+245); chr17:38347616-38347753/WIPF2 (−27889), RAPGEFL1 (+13443); chr17:38443873-38443912/CDC
6 (−253); chr17:38511540-38511587/GJD3 (+9381), RARA (+46141); chr17:38935823-38935956/KRT26 (−7479),
KRT27 (+2896); chr17:39992508-39992533/KLHL10 (−1522), NT5C3L (+2); chr17:40021442-40021478/KLHL
11 (+169); chr17:40427984-40428103/STAT5B (+380); chr17:40441048-40441131/STAT5A (+1525), STAT3
(+99423); chr17:40441367-40441512/STAT5A (+1875), STAT3 (+99073); chr17:40558167-40558226/STAT3 (−17
684), PTRF (+17141); chr17:40574841-40574882/PTRF (+476); chr17:40684312-40684353/NAGLU (−3618); ch
r17:40822250-40822496/PLEKHH3 (+6675), TUBG2 (+11107); chr17:40825694-40825828/PLEKHH3 (+3287),
TUBG2 (+14495); chr17:40832283-40832365/PLEKHH3 (−3276), CNTNAP1 (−2308); chr17:40932823-4093284
3/WNK4 (+184); chr17:40935970-40936006/WNK4 (+3339), CCDC56 (+14716); chr17:40937395-40937445/W
NK4 (+4771), CCDC56 (+13284); chr17:41174027-41174068/RND2 (−3210), VAT1 (+411); chr17:41791024-417
91085/MEOX1 (−52124), SOST (+45101); chr17:41791344-41791412/MEOX1 (−52447), SOST (+44778); chr17:
41832697-41832729/MEOX1 (−93782), SOST (+3443); chr17:41832872-41833144/MEOX1 (−94077), SOST (+3
148); chr17:41910679-41910700/MPP3 (−143); chr17:41984347-41984363/MPP2 (+758); chr17:41994796-4199
4950/FAM215A (+297); chr17:42030751-42030779/PPY (−10932), PYY (+51072); chr17:42072534-42072599/P
PY (−52734), PYY (+9270); chr17:42264045-42264076/TMUB2 (−293); chr17:42386383-42386418/RUNDC3A
(+474); chr17:42392411-42392465/RUNDC3A (+6511), SLC25A39 (+9779); chr17:42392756-42392796/RUNDC3
A (+6849), SLC25A39 (+9441); chr17:42401501-42401544/SLC25A39 (+694); chr17:42404284-42404323/SLC
25A39 (−2087); chr17:42463324-42463393/FAM171A2 (−22124), ITGA2B (+3514); chr17:42734444-42734502/
DBF4B (−51503), FZD2 (+99661); chr17:42767481-42767556/DBF4B (−18457), FZD2 (+132707); chr17:428357
90-42836011/ADAM11 (−667); chr17:42907631-42907654/GJC1 (+536); chr17:42988995-42989016/GFAP (+39
14), FAM187A (+8879); chr17:43037189-43037325/KIF18B (−12175), C1QL1 (+8387); chr17:43044807-430448
63/C1QL1 (+809); chr17:43047404-43047752/C1QL1 (−1934); chr17:43099558-43099609/C1QL1 (−53940), DC
AKD (+29394); chr17:43198566-43198714/PLCD3 (+11251), NMT1 (+59960); chr17:43221186-43221214/HEXI
M1 (−3484); chr17:43221463-43221574/HEXIM1 (−3165); chr17:43238829-43238870/HEXIM2 (+586); chr17:43
298986-43298996/FMNL1 (−301); chr17:43299291-43299306/FMNL1 (+7); chr17:43324709-43324795/FMNL1
(+25460), MAP3K14 (+69662); chr17:43339408-43339475/FMNL1 (+40150), MAP3K14 (+54972); chr17:435098
42-43509907/ARHGAP27 (−6863), ARHGAP27 (+1237); chr17:43578940-43578973/CRHR1 (−282689), PLEKH
M1 (−10811); chr17:43678205-43678229/CRHR1 (−183429), PLEKHM1 (−110071); chr17:43860818-43860866/
CRHR1 (−804); chr17:43862960-43863021/IMP5 (−59265), CRHR1 (+1345); chr17:43974400-43974472/STH (−1
02180), MAPT (+2688); chr17:43974607-43974621/STH (−102002), MAPT (+2866); chr17:43974831-43974900/
STH (−101750), MAPT (+3118); chr17:44846935-44847043/WNT3 (+49093), NSF (+178954); chr17:44848970-
44848999/WNT3 (+47097), NSF (+180950); chr17:44849801-44849831/WNT3 (+46266), NSF (+181781); chr1
7:44928456-44928485/WNT9B (−497); chr17:44929235-44929255/WNT9B (+277); chr17:44942587-44942694/
GOSR2 (−57845), WNT9B (+13673); chr17:45055801-45055831/RPRML (+798); chr17:45354370-45354488/C1
7orf57 (−46898), ITGB3 (+23221); chr17:45501244-45501325/NPEPPS (−107159), C17orf57 (+99958); chr17:45
726965-45727032/KPNB1 (−276); chr17:45728121-45728131/KPNB1 (+851); chr17:45771124-45771151/TBKB
P1 (−1492); chr17:45772723-45772799/TBKBP1 (+131); chr17:45811758-45811877/TBX21 (+1208), OSBPL7
(+87329); chr17:45918203-45918255/SCRN2 (+470); chr17:45918620-45918690/SCRN2 (+44); chr17:4592468
1-45924816/SCRN2 (−6050), SP6 (+8491); chr17:45924921-45924988/SCRN2 (−6256), SP6 (+8285); chr17:459
28090-45928129/SCRN2 (−9411), SP6 (+5130); chr17:46124970-46125006/NFE2L1 (−698); chr17:46178523-46
178576/CBX1 (+10); chr17:46233723-46233750/SNX11 (+48817), SKAP1 (+273857); chr17:46604392-466045
59/SKAP1 (−96882), HOXB1 (+3796); chr17:46618948-46619087/HOXB1 (−10746), HOXB2 (+3375); chr17:466
19244-46619451/HOXB1 (−11076), HOXB2 (+3045); chr17:46619834-46619851/HOXB1 (−11571), HOXB2 (+25
50); chr17:46622011-46622061/HOXB2 (+357); chr17:46645993-46646011/HOXB2 (−23609), HOXB3 (+5808);
chr17:46647838-46647916/HOXB2 (−25484), HOXB3 (+3933); chr17:46654127-46654153/HOXB3 (−2330); chr1
7:46655419-46655450/HOXB3 (−3625), HOXB4 (+308); chr17:46655999-46656057/HOXB3 (−4218), HOXB4 (−2
85); chr17:46659274-46659384/HOXB4 (−3586); chr17:46659926-46659966/HOXB4 (−4203); chr17:46663825-4
6663855/HOXB4 (−8097), HOXB5 (+7263); chr17:46669646-46669780/HOXB4 (−13970), HOXB5 (+1390); chr1
7:46670028-46670083/HOXB4 (−14313), HOXB5 (+1047); chr17:46673603-46673646/HOXB5 (−2522); chr17:46
690364-46690429/HOXB7 (−2014); chr17:46690705-46690852/HOXB7 (−2396); chr17:46691819-46691987/HO
XB7 (−3520), HOXB8 (+398); chr17:46697019-46697064/HOXB8 (−4741); chr17:46711155-46711236/HOXB9
(−7361), PRAC (+88686); chr17:46711417-46711481/HOXB9 (−7614), PRAC (+88433); chr17:46796374-4679644
1/HOXB9 (−92573), PRAC (+3474); chr17:46796638-46796849/HOXB9 (−92909), PRAC (+3138); chr17:467972
14-46797274/HOXB9 (−93409), PRAC (+2638); chr17:46801048-46801108/PRAC (−1196); chr17:46802912-468
02993/PRAC (−3071); chr17:46811500-46811594/HOXB13 (−5436), TTLL6 (+82922); chr17:46824276-4682435
8/HOXB13 (−18206), TTLL6 (+70152); chr17:46827502-46827625/HOXB13 (−21453), TTLL6 (+66905); chr17:46
830136-46830189/HOXB13 (−24052), TTLL6 (+64306); chr17:46832326-46832489/HOXB13 (−26297), TTLL6
(+62061); chr17:46915331-46915388/ATP5G1 (−54788), CALCOCO2 (+6988); chr17:46985597-46985658/UBE2
Z (−103); chr17:47073029-47073103/IGF2BP1 (−1708); chr17:47073346-47073369/IGF2BP1 (−1416); chr17:470
75735-47075879/B4GALNT2 (−134522), IGF2BP1 (+1033); chr17:47297075-47297113/GNGT2 (−9158), PHOS
PHO1 (+11034); chr17:47301239-47301286/GNGT2 (−13327), PHOSPHO1 (+6865); chr17:47301766-4730180
1/GNGT2 (−13848), PHOSPHO1 (+6344); chr17:47302258-47302426/GNGT2 (−14406), PHOSPHO1 (+5786); c
hr17:47547697-47547746/PHB (−55480), NGFR (−24933); chr17:47647876-47647981/NXPH3 (−5369), NGFR
(+75274); chr17:47841531-47841693/SLC35B1 (−56330), KAT7 (−24369); chr17:47940278-47940304/DLX4 (−106
271), TAC4 (−14912); chr17:47968071-47968095/DLX4 (−78479), TAC4 (−42704); chr17:48041890-48041904/D
LX4 (−4665); chr17:48042141-48042213/DLX4 (−4385); chr17:48042648-48042750/DLX4 (−3863); chr17:480469
80-48047056/DLX4 (+456); chr17:48071705-48071806/DLX3 (+832); chr17:48224717-48224860/PPP1R9B (+3
088), PDK2 (+52150); chr17:48277451-48277483/HILS1 (−27630), COL1A1 (+1533); chr17:48351499-4835161
7/XYLT2 (−71835), TMEM92 (+2791); chr17:48545982-48546068/CHAD (+202); chr17:48546192-48546385/CH
AD (−62); chr17:48546681-48546850/CHAD (−539); chr17:48619291-48619346/SPATA20 (−5110), EPN3 (+9271);
chr17:48691189-48691285/ABCC3 (−20981), CACNA1G (+52788); chr17:48997107-48997172/TOB1 (−53425),
SPAG9 (+201086); chr17:49337431-49337456/UTP18 (−453), MBTD1 (−17); chr17:50235259-50235416/CA10
(+1823), UTP18 (+897441); chr17:52978428-52978453/TOM1L1 (+389); chr17:53341705-53341786/HLF (−575);
chr17:54991753-54991828/TRIM25 (−382); chr17:55123060-55123208/AKAP1 (−39419), SCPEP1 (+67666); c
hr17:55123547-55123602/AKAP1 (−38978), SCPEP1 (+68107); chr17:55123723-55123809/AKAP1 (−38787), S
CPEP1 (+68298); chr17:55333085-55333144/MSI2 (−816); chr17:55951994-55952035/MRPS23 (−24582), VEZF
1 (+113600); chr17:56161224-56161247/DYNLL2 (+456); chr17:56234878-56234937/OR4D2 (−12109), OR4D1
(+2393); chr17:56326751-56326781/LPO (+10979), MPO (+31530); chr17:56405140-56405255/BZRAP1 (+954);
chr17:56410487-56410659/BZRAP1 (−4421); chr17:56564968-56565373/HSF5 (+588); chr17:56565511-5656
5531/HSF5 (+238); chr17:56592027-56592050/HSF5 (−26280), MTMR4 (+3212); chr17:56834001-56834019/P
PM1E (+778); chr17:57183731-57183806/TRIM37 (+497); chr17:57642657-57642736/DHX40 (−189); chr17:581
03146-58103169/RNFT1 (−61041), HEATR6 (+53134); chr17:58216303-5821 6565/AP1S2 (−36154), CA4 (−1086
8); chr17:58216837-58216865/AP1S2 (−36571), CA4 (−10451); chr17:58217299-58217356/AP1S2 (−37048), CA
4 (−9974); chr17:58227411-58227434/CA4 (+121); chr17:59473504-59473558/TBX2 (−3726); chr17:59473671-5
9473906/TBX2 (−3468); chr17:59474441-59474484/TBX2 (−2794); chr17:59478889-59478957/TBX4 (−54884), T
BX2 (+1666); chr17:59480396-59480516/TBX4 (−53351), TBX2 (+3199); chr17:59480855-59480953/TBX4 (−52
903), TBX2 (+3647); chr17:59481679-59481798/TBX4 (−52068), TBX2 (+4482); chr17:59482168-59482245/TB
X4 (−51600), TBX2 (+4950); chr17:59482595-59482693/TBX4 (−51163), TBX2 (+5387); chr17:59485687-59485
778/TBX4 (−48074), TBX2 (+8476); chr17:59487527-59487608/TBX4 (−46239), TBX2 (+10311); chr17:5948912
9-59489181/TBX4 (−44652), TBX2 (+11898); chr17:59529151-59529253/TBX4 (−4605); chr17:59529390-59529
843/TBX4 (−4190); chr17:59532042-59532269/TBX4 (−1651); chr17:59532518-59532591/TBX4 (−1252); chr17:5
9533768-59533874/TBX4 (+14); chr17:59534406-59534556/TBX4 (+674); chr17:60704362-60704402/MRC2
(−380); chr17:61512642-61512695/CYB561 (+10876), TANC2 (+425771); chr17:61516023-61516064/CYB561
(+7501), TANC2 (+429146); chr17:61524163-61524220/CYB561 (−647); chr17:61777667-61777721/LIMD2 (−175);
chr17:61778074-61778106/LIMD2 (−571); chr17:61778249-61778423/LIMD2 (−817); chr17:61778549-6177871
8/LIMD2 (−1115); chr17:61850799-61850823/DDX42 (−756), CCDC47 (+277); chr17:61926325-61926507/SMA
RCD2 (−6065), CSH2 (+24673); chr17:61926625-61926759/SMARCD2 (−6341), CSH2 (+24397); chr17:6203898
0-62039019/CD79B (−29296), SCN4A (+11278); chr17:62097917-62098020/ICAM2 (+25); chr17:62208165-622
08238/ERN1 (−700); chr17:62340247-62340352/TEX2 (+353); chr17:62492984-62493015/POLG2 (+184); chr17:
62657954-62658003/SMURF2 (+407); chr17:62658493-62658507/SMURF2 (−114); chr17:62658776-62658830/
SMURF2 (−417); chr17:62774545-62774587/SMURF2 (−116180), LOC146880 (+3056); chr17:62774784-62774
871/SMURF2 (−116442), LOC146880 (+2794); chr17:62775015-62775170/SMURF2 (−116707), LOC146880 (+2
529); chr17:62775845-62775864/SMURF2 (−117469), LOC146880 (+1767); chr17:62776423-62776446/SMURF
2 (−118049), LOC146880 (+1187); chr17:62972120-62972213/LRRC37A3 (−57179), GNA13 (+80753); chr17:63
052281-63052314/GNA13 (+622); chr17:64447706-64447819/CACNG5 (−425585), PRKCA (+148837); chr17:6
4962267-64962287/CACNG1 (−78375), CACNG4 (+1297); chr17:64962513-64962592/CACNG1 (−78099), CAC
NG4 (+1573); chr17:65266480-65266608/HELZ (−25225), PSMD12 (+96177); chr17:65821526-65821565/BPTF
(−234); chr17:65989821-65989837/KPNA2 (−42019), BPTF (+168049); chr17:66097351-66097376/AMZ2 (−146
781), KPNA2 (+65516); chr17:66097977-66098022/AMZ2 (−146145), KPNA2 (+66152); chr17:66193039-66193
079/AMZ2 (−51086), KPNA2 (+161211); chr17:66194561-66194593/AMZ2 (−49568), KPNA2 (+162729); chr17:6
6194749-66194824/AMZ2 (−49358), KPNA2 (+162939); chr17:66201212-66201233/AMZ2 (−42922), KPNA2 (+1
69375); chr17:66243589-66243743/AMZ2 (−479); chr17:66243856-66243888/AMZ2 (−273); chr17:66244393-66
244456/AMZ2 (+280); chr17:66308870-66309006/SLC16A6 (−21533), WIPI1 (+144715); chr17:66508805-66508
832/PRKAR1A (+709); chr17:66509087-66509117/PRKAR1A (+992); chr17:66597515-66597560/FAM20A (−44
3); chr17:67323767-67323835/ABCA5 (−478); chr17:70112164-70112352/SOX9 (−4903); chr17:70113567-7011
3647/SOX9 (−3554); chr17:70114019-70114152/SOX9 (−3075); chr17:70119110-70119135/SOX9 (+1962), SLC
39A11 (+969730); chr17:70119553-70119570/SOX9 (+2401), SLC39A11 (+969291); chr17:70119837-7011994
4/SOX9 (+2730), SLC39A11 (+968962); chr17:70120106-70120133/SOX9 (+2959), SLC39A11 (+968733); chr1
7:70216307-70216392/SOX9 (+99189), SLC39A11 (+872503); chr17:70379270-70379341/SOX9 (+262145), S
LC39A11 (+709547); chr17:70636836-70636875/SLC39A11 (+451997), SOX9 (+519695); chr17:71161159-711
61183/SSTR2 (+11); chr17:71227501-71227555/C17orf80 (−1248); chr17:71398132-71398201/CDC42EP4 (−90
024), SDK2 (+242060); chr17:71736508-71736595/RPL38 (−463243), SDK2 (−96325); chr17:71898164-718982
28/RPL38 (−301599), SDK2 (−257969); chr17:72281096-72281163/KIF19 (−41221), DNAI2 (+10744); chr17:723
22558-72322611/KIF19 (+234); chr17:72322790-72322818/KIF19 (+453); chr17:72353260-72353416/BTBD17
(+4620), KIF19 (+30987); chr17:72450313-72450330/CD300A (−12200), GPRC5C (+20486); chr17:72462521-72
462598/CD300A (+38); chr17:72667482-72667681/CD300E (−47685), CD300LF (+41526); chr17:72754588-727
54712/SLC9A3R1 (+9887), NAT9 (+17820); chr17:72755637-72755752/SLC9A3R1 (+10932), NAT9 (+16775);
chr17:72839288-72839323/GRIN2C (+16701), TMEM104 (+66684); chr17:72848460-72848486/GRIN2C (+753
4), TMEM104 (+75851); chr17:72848851-72848870/GRIN2C (+7146), TMEM104 (+76239); chr17:72869671-72
869734/FDXR (−547); chr17:72889357-72889430/FADS6 (+311); chr17:72931948-72932011/OTOP3 (+83); chr
17:73073684-73073928/SLC16A5 (−10249), KCTD2 (+30527); chr17:73110635-73110676/ARMC7 (+4574), NT
5C (+17234); chr17:73127747-73127802/NT5C (+115); chr17:73177807-73177866/HN1 (−27062), SUMO2 (+12
61); chr17:73286126-73286178/SLC25A19 (−622); chr17:73401627-73401658/GRB2 (+147); chr17:73512219-7
3512283/CASKIN2 (−624), TSEN54 (−358); chr17:73521178-73521238/LLGL2 (−575); chr17:73522427-7352248
2/LLGL2 (+672); chr17:73532558-73532630/MYO15B (−51545), LLGL2 (+10811); chr17:73584107-73584160/M
YO15B (−5); chr17:73584394-73584657/MYO15B (+387); chr17:73749592-73749684/GALK1 (+11642), ITGB4
(+32122); chr17:73749813-73750133/GALK1 (+11307), ITGB4 (+32457); chr17:73760840-73760881/GALK1 (+4
19); chr17:73851279-73851335/WBP2 (+194); chr17:73873681-73873726/TRIM47 (+952); chr17:73900929-73
900950/MRPL38 (+241); chr17:74070480-74070709/ZACN (−4668), SRP68 (−1988), GALR2 (−297); chr17:7407
1978-74072049/SRP68 (−3407), ZACN (−3249); chr17:74072711-74072998/SRP68 (−4248), ZACN (−2408); chr1
7:74118061-74118117/EXOC7 (−18221), FOXJ1 (+19291); chr17:74118235-74118360/EXOC7 (−18430), FOXJ
1 (+19082); chr17:74126709-74126808/EXOC7 (−26891), FOXJ1 (+10621); chr17:74136212-74136244/EXOC7
(−36360), FOXJ1 (+1152); chr17:74136442-74136570/FOXJ1 (+874); chr17:74141516-74141579/FOXJ1 (−4168);
chr17:74235881-74235976/RNF157 (+461); chr17:74349121-74349164/RNF157 (−112753), PRPSAP1 (+108
7); chr17:74350082-74350131/PRPSAP1 (+123); chr17:74533558-74533674/PRCD (−2505), CYGB (+166); chr
17:74534363-74534387/PRCD (−1746), CYGB (−593); chr17:74553278-74553405/PRCD (+17221), ST6GALNA
C2 (+28803); chr17:74564967-74565084/ST6GALNAC2 (+17119), PRCD (+28905); chr17:74668194-74668251/
ST6GALNAC1 (−28329), MXRA7 (+38833); chr17:74722474-74722507/METTL23 (−421), JMJD6 (+390); chr17:
74868861-74868886/SEC14L1 (−268131), MGAT5B (+4076); chr17:74994453-74994500/SEC14L1 (−142528),
MGAT5B (+129679); chr17:75033184-75033232/SEC14L1 (−103797), MGAT5B (+168410); chr17:75369458-75
369492/TNRC6C (−630843), SEPT9 (+91983); chr17:75370116-75370185/TNRC6C (−630167), SEPT9 (+92659);
chr17:75372359-75372419/TNRC6C (−627929), SEPT9 (+94897); chr17:75386077-75386103/TNRC6C (−614
228), SEPT9 (+108598); chr17:75693115-75693216/TNRC6C (−307152), SEPT9 (+415674); chr17:76113692-7
6113738/TMC6 (+11146), TNRC6C (+113397); chr17:76127314-76127425/TMC6 (−2509), TMC8 (+511); chr17:
76127527-76127551/TMC6 (−2678), TMC8 (+680); chr17:76127922-76128044/TMC6 (−3122), TMC8 (+1124); c
hr17:76128664-76128693/TMC6 (−3818), TMC8 (+1820); chr17:76133903-76133936/C17orf99 (−8514), TMC8
(+7061); chr17:76228162-76228213/TMEM235 (+797); chr17:76254727-76254836/TMEM235 (+27391), SOCS3
(+101376); chr17:76259469-76259526/TMEM235 (+32107), SOCS3 (+96660); chr17:76275001-76275113/TM
EM235 (+47666), SOCS3 (+81101); chr17:76356583-76356613/SOCS3 (−440); chr17:76380936-76380981/PG
S1 (+6224), DNAH17 (+192517); chr17:76771016-76771064/DNAH17 (−197564), CYTH1 (+7336); chr17:76836
688-76836714/USP36 (+268); chr17:76921355-76921422/TIMP2 (+83); chr17:76921806-76921859/TIMP2 (−36
1); chr17:77020266-77020313/C1QTNF1 (+39); chr17:77178872-77178962/ENGASE (+107898), RBFOX3 (+29
9763); chr17:77179064-77179081/ENGASE (+108054), RBFOX3 (+299607); chr17:77179330-77179380/ENGA
SE (+108336), RBFOX3 (+299325); chr17:77179483-77179617/ENGASE (+108531), RBFOX3 (+299130); chr1
7:77179777-77179799/ENGASE (+108769), RBFOX3 (+298892); chr17:77397120-77397189/RBFOX3 (+81525),
ENGASE (+326136); chr17:77721549-77721582/CBX2 (−30411), ENPP7 (+16684); chr17:77721756-7772203
8/CBX2 (−30080), ENPP7 (+17015); chr17:77722215-77722442/CBX2 (−29648), ENPP7 (+17447); chr17:77751
046-77751068/CBX2 (−920); chr17:77752269-77752326/CBX2 (+321); chr17:77753177-77753284/CBX2 (+125
4), CBX8 (+17684); chr17:77763481-77763545/CBX8 (+7402), CBX2 (+11536); chr17:77764786-77764828/CB
X8 (+6108), CBX2 (+12830); chr17:77776996-77777052/CBX8 (−6109), CBX4 (+36189); chr17:77777904-7777
7943/CBX8 (−7009), CBX4 (+35289); chr17:77788346-77788400/CBX8 (−17458), CBX4 (+24840); chr17:77788
883-77788911/CBX8 (−17982), CBX4 (+24316); chr17:77789296-77789473/CBX8 (−18470), CBX4 (+23828); ch
r17:77789587-77789717/CBX8 (−18737), CBX4 (+23561); chr17:77805979-77805997/CBX8 (−35073), CBX4
(+7225); chr17:77806339-77806352/CBX8 (−35431), CBX4 (+6867); chr17:77814169-77814364/CBX4 (−1054); ch
r17:77815985-77816033/CBX4 (−2796); chr17:77916814-77916886/CBX4 (−103637), TBC1D16 (+92797); chr17:
78010753-78010812/TBC1D16 (−1136), CCDC40 (+352); chr17:78120623-78120666/EIF4A3 (+337); chr17:78
450937-78450959/NPTX1 (−544); chr17:78451954-78452108/NPTX1 (−1627); chr17:78955176-78955204/CHM
P6 (−10451), RPTOR (+436565); chr17:78977613-78977693/BAIAP2 (−31294), CHMP6 (+12012); chr17:789923
73-78992454/BAIAP2 (−16533), CHMP6 (+26773); chr17:79007416-79007488/BAIAP2 (−1495); chr17:79125227-
79125258/AATK (+14629), BAIAP2 (+116296); chr17:79134164-79134195/AATK (+5692), BAIAP2 (+125233);
chr17:79134688-79134748/AATK (+5154), BAIAP2 (+125771); chr17:79139981-79140028/AATK (−133); chr17:
79192402-79192482/AATK (−52570), AZI1 (+4309); chr17:79259009-79259050/SLC38A10 (+10066), C17orf89
(+45919); chr17:79315066-79315125/BAHCC1 (−58444), TMEM105 (−10622); chr17:79315417-79315511/BAH
CC1 (−58076), TMEM105 (−10990); chr17:79315615-79315782/BAHCC1 (−57841), TMEM105 (−11225); chr17:7
9316389-79316427/BAHCC1 (−57132), TMEM105 (−11934); chr17:79317101-79317178/BAHCC1 (−56400), TM
EM105 (−12666); chr17:79370667-79370707/BAHCC1 (−2853); chr17:79387021-79387056/BAHCC1 (+13499),
ACTG1 (+92853); chr17:79405398-79405477/BAHCC1 (+31898), ACTG1 (+74454); chr17:79420883-79420919/
BAHCC1 (+47361), ACTG1 (+58991); chr17:79450366-79450417/ACTG1 (+29500), BAHCC1 (+76852); chr17:
79450990-79451034/ACTG1 (+28880), BAHCC1 (+77472); chr17:79504021-79504047/FSCN2 (+8617), C17orf
70 (+15395); chr17:79614999-79615035/TSPAN10 (+5684), PDE6G (+8590); chr17:79615325-79615434/TSPA
N10 (+6047), PDE6G (+8227); chr17:79620320-79620357/PDE6G (+3268), TSPAN10 (+11006); chr17:796204
64-79620575/PDE6G (+3087), TSPAN10 (+11187); chr17:79650823-79650872/HGS (−114), ARL16 (+106); chr
17:79707714-79707780/GCGR (−54263), SLC25A10 (+28376); chr17:79761813-79761833/GCGR (−187); chr17:
79762870-79762898/GCGR (+874); chr17:79790751-79790787/P4HB (+27775), GCGR (+28759); chr17:7981
8183-79818230/P4HB (+337); chr17:79882016-79882056/MAFG (−592); chr17:79919509-79919572/NOTUM
(−484); chr17:79919891-79919913/NOTUM (−845); chr17:79935345-79935368/ASPSCR1 (−69); chr17:79988144-
79988226/RAC3 (−1347); chr17:79989846-79989868/RAC3 (+325); chr17:79990232-79990262/RAC3 (+715); c
hr17:80057399-80057502/FASN (−1345); chr17:80064159-80064207/SLC16A3 (−122099), FASN (−8077); chr17:
80085286-80085323/SLC16A3 (−100977), FASN (−29199); chr17:80188628-80188673/SLC16A3 (+2369), CSN
K1D (+42943); chr17:80250237-80250279/CSNK1D (−18664), CD7 (+25222); chr17:80272852-80272918/CSNK
1D (−41291), CD7 (+2595); chr17:80273790-80273862/CSNK1D (−42232), CD7 (+1654); chr17:80291972-8029
2003/SECTM1 (−67); chr17:80330001-80330164/UTS2R (−2118); chr17:80332826-80332928/UTS2R (+676); ch
r17:80333241-80333309/HEXDC (−42977), UTS2R (+1074); chr17:80477829-80477847/FOXK2 (+244); chr17:8
0943958-80944089/ZNF750 (−146093), B3GNTL1 (+65662); chr17:81027192-81027252/B3GNTL1 (−17536), M
ETRNL (−10345); chr17:81037295-81037327/METRNL (−256); chr17:81038844-81038865/METRNL (+1288); ch
r17:81039672-81039746/METRNL (+2142); chr17:81147427-81147479/METRNL (+109886); chr18:597185-597
228/CLUL1 (−19493), CETN1 (+16838); chr18:712732-712794/ENOSF1 (−101); chr18:712960-712987/ENOSF1
(−312); chr18:905616-905692/ADCYAP1 (+357); chr18:907694-907721/ADCYAP1 (+2411); chr18:908271-9082
99/ADCYAP1 (+2988); chr18:909085-909183/ADCYAP1 (+3837); chr18:909289-909318/ADCYAP1 (+4007); ch
r18:2905921-2906054/EMILIN2 (+58960), LPIN2 (+105957); chr18:4454691-4454777/DLGAP1 (+532); chr18:4
455031-4455258/DLGAP1 (+121); chr18:4455418-4455538/DLGAP1 (−212); chr18:5196259-5196575/DLGAP1
(−741151), ZFP161 (+99777); chr18:5197272-5197329/DLGAP1 (−742035), ZFP161 (+98893); chr18:5543158-5
543337/EPB41L3 (+738); chr18:5543752-5543899/EPB41L3 (+160); chr18:5629484-5629569/EPB41L3 (−8554
1), TMEM200C (+262576); chr18:5629829-5629999/EPB41L3 (−85928), TMEM200C (+262189); chr18:5630655-
5630693/EPB41L3 (−86688), TMEM200C (+261429); chr18:5869021-5869147/EPB41L3 (−325098), TMEM200
C (+23019); chr18:5890337-5890541/EPB41L3 (−346453), TMEM200C (+1664); chr18:5890847-5891242/EPB4
1L3 (−347059), TMEM200C (+1058); chr18:5891421-5891443/TMEM200C (+671); chr18:5894946-5895017/TM
EM200C (−2879); chr18:6414127-6414203/L3MBTL4 (+745); chr18:6414445-6414507/L3MBTL4 (+434); chr18:
7116331-7116449/LAMA1 (+1423), ARHGAP28 (+281958); chr18:7116883-7117059/LAMA1 (+842); chr18:711
7320-7117374/LAMA1 (+466); chr18:7117560-7117615/LAMA1 (+225); chr18:7118091-7118131/LAMA1 (−298);
chr18:7566756-7566780/PTPRM (−546); chr18:7568718-7568792/PTPRM (+1441); chr18:8367459-8367503/R
AB12 (−241962), PTPRM (+800167); chr18:8608838-8608901/RAB12 (−573); chr18:8705763-8705789/NDUFV2
(−396852), RAB12 (+96333); chr18:9136324-9136383/ANKRD12 (−397); chr18:9136517-9136541/ANKRD12
(−222); chr18:9333896-9333927/TWSG1 (−853); chr18:9613780-9613823/PPP4R1 (+798); chr18:10032410-1003
2552/APCDD1 (−422144), VAPA (+118526); chr18:10032685-10032841/APCDD1 (−421862), VAPA (+118808);
chr18:10726296-10726413/NAPG (+200482), PIEZO2 (+422406); chr18:11148625-11148741/PIEZO2 (+78); ch
r18:11149094-11149115/PIEZO2 (−344); chr18:11149274-11149298/PIEZO2 (−525); chr18:11149760-11149779/
PIEZO2 (−1009); chr18:11752007-11752075/CHMP1B (−99348), GNAL (+62905); chr18:11752605-11752640/C
HMP1B (−98766), GNAL (+63487); chr18:12246920-12247054/CIDEA (−7331), ANKRD62 (+144282); chr18:122
54147-12254304/CIDEA (−92); chr18:12254856-12254902/CIDEA (+561); chr18:12307506-12307602/TUBB6
(−703); chr18:12408323-12408339/SLMO1 (+436); chr18:12420804-12420854/SLMO1 (+12934), SPIRE1 (+2370
83); chr18:12911221-12911577/SEH1L (−36584), PTPN2 (−27065); chr18:13136145-13136192/C18orf1 (−82617),
CEP192 (+144808); chr18:13217094-13217130/C18orf1 (−1674); chr18:13611408-13611480/FAM210A (+115
147), C18orf1 (+392658); chr18:13611980-13612020/FAM210A (+114591), C18orf1 (+393214); chr18:1386892
0-13868946/MC5R (+43390), MC2R (+46602); chr18:14450449-14450486/ZNF519 (−317979), ANKRD30B (−29
7771); chr18:14748108-14748450/ANKRD30B (+40); chr18:14999428-14999468/ANKRD30B (+251209); chr18:
15197677-15197744/ANKRD30B (+449472); chr18:15198278-15198299/ANKRD30B (+450050); chr18:191796
98-19179728/ESCO1 (+980); chr18:19179897-19179961/ESCO1 (+764); chr18:19284709-19284753/ABHD3
(+35); chr18:19745475-19745613/GATA6 (−3872); chr18:19745725-19745928/GATA6 (−3589); chr18:19746239-1
9746384/GATA6 (−3104); chr18:19747083-19747141/GATA6 (−2304); chr18:19751836-19751869/GATA6 (+243
7), CTAGE1 (+246025); chr18:19756471-19756539/GATA6 (+7089), CTAGE1 (+241373); chr18:19756876-197
56983/GATA6 (+7514), CTAGE1 (+240948); chr18:19929051-19929116/CTAGE1 (+68794), GATA6 (+179668);
chr18:20512838-20512902/RBBP8 (−425); chr18:21851525-21851561/OSBPL1A (+126290), CABYR (+132588);
chr18:22929096-22929186/ZNF521 (+3073), HRH4 (+888548); chr18:22929798-22929820/ZNF521 (+2405),
HRH4 (+889216); chr18:24130947-24131098/KCTD1 (−2523); chr18:24765188-24765230/CHST9 (+80); chr18:
25755452-25755555/CHST9 (−990215), CDH2 (+1941); chr18:25757972-25758128/CDH2 (−605); chr18:286209
56-28621033/DSC3 (+1786); chr18:28622998-28623039/DSC3 (−238); chr18:28682246-28682286/DSC2 (+122);
chr18:29672872-29672905/RNF138 (+320); chr18:31739642-31739742/NOL4 (+63823), ASXL3 (+581151); c
hr18:31803660-31803673/NOL4 (−152); chr18:32074321-32074337/NOL4 (−270814), DTNA (−261611); chr18:3
3529960-33529996/SLC39A6 (+179379), GALNT1 (+295445); chr18:33709047-33709083/ELP2 (−772), SLC39
A6 (+292); chr18:33877151-33877206/FHOD3 (−523); chr18:33962286-33962330/FHOD3 (+84606), TPGS2
(+446850); chr18:34823778-34823862/TPGS2 (−414662), CELF4 (+322180); chr18:34833554-34833582/TPGS2
(−424410), CELF4 (+312432); chr18:34833767-34833937/TPGS2 (−424694), CELF4 (+312148); chr18:34854793-
34854858/TPGS2 (−445668), CELF4 (+291174); chr18:35065431-35065455/TPGS2 (−656285), CELF4 (+8055
7); chr18:35104551-35104934/TPGS2 (−695585), CELF4 (+41257); chr18:35144215-35144277/TPGS2 (−73508
8), CELF4 (+1754); chr18:35144937-35144968/TPGS2 (−735795), CELF4 (+1047); chr18:35146037-35146061/
CELF4 (−49); chr18:35146765-35146895/CELF4 (−830); chr18:35147169-35147245/CELF4 (−1207); chr18:3514
7804-35147834/CELF4 (−1819); chr18:44335851-44335917/LOXHD1 (−98888), ST8SIA5 (+1155); chr18:44336
489-44336523/ST8SIA5 (+533); chr18:44336763-44336788/ST8SIA5 (+263); chr18:44337618-44337649/ST8SI
A5 (−595); chr18:44338021-44338079/ST8SIA5 (−1011); chr18:44497277-44497343/PIAS2 (+185); chr18:44677
031-44677055/HDHD2 (−172); chr18:44773117-44773466/IER3IP1 (−70547), SKOR2 (+2262); chr18:44773970-
44774081/IER3IP1 (−71281), SKOR2 (+1528); chr18:44777792-44777862/SKOR2 (−2273); chr18:44787412-44
787491/SKOR2 (−11898), SMAD2 (+670063); chr18:44788869-44788908/SKOR2 (−13335), SMAD2 (+668626);
chr18:44790115-44790144/SKOR2 (−14576), SMAD2 (+667385); chr18:44790474-44790495/SKOR2 (−14931),
SMAD2 (+667030); chr18:45058293-45058307/SKOR2 (−282746), SMAD2 (+399215); chr18:45456613-454566
77/SMAD2 (+870); chr18:45534647-45534675/SMAD2 (−77146), ZBTB7C (+129019); chr18:45934223-4593431
7/ZBTB7C (−270590), CTIF (−131157); chr18:45934467-45934609/ZBTB7C (−270858), CTIF (−130889); chr18:4
6065605-46065659/CTIF (+205); chr18:46307640-46307700/SMAD7 (+169411), CTIF (+242243); chr18:46361
269-46361322/SMAD7 (+115785), CTIF (+295869); chr18:47340158-47340233/ACAA2 (+55); chr18:47794670-
47794700/CCDC11 (−1820); chr18:47794875-47794919/CCDC11 (−2032); chr18:48405502-48405525/ME2 (+8
2); chr18:49866360-49866411/DCC (−156); chr18:53447481-53447528/TCF4 (−144317), TXNL1 (+858765); chr
18:53447772-53447798/TCF4 (−144597), TXNL1 (+858485); chr18:55019929-55020166/ST8SIA3 (+327); chr18:
55020699-55020804/ONECUT2 (−82165), ST8SIA3 (+1031); chr18:55020945-55020965/ONECUT2 (−81962),
ST8SIA3 (+1234); chr18:55095157-55095278/ONECUT2 (−7699), ST8SIA3 (+75497); chr18:55095498-550956
93/ONECUT2 (−7321), ST8SIA3 (+75875); chr18:55095912-55096113/ONECUT2 (−6904), ST8SIA3 (+76292); c
hr18:55101991-55102007/ONECUT2 (−918); chr18:55103414-55103761/ONECUT2 (+671); chr18:55103881-55
103897/ONECUT2 (+972); chr18:55104141-55104166/ONECUT2 (+1237), FECH (+149815); chr18:55107309-
55107361/ONECUT2 (+4418), FECH (+146634); chr18:55107885-55107937/ONECUT2 (+4994), FECH (+1460
58); chr18:55253592-55253637/FECH (+354); chr18:55470841-55470898/ATP8B1 (−543); chr18:56028818-560
28858/ALPK2 (+267351), NEDD4L (+317228); chr18:56338879-56338915/MALT1 (+279); chr18:56530991-565
31013/ZNF532 (+941); chr18:56531194-56531228/SEC11C (−275914), ZNF532 (+1150); chr18:56806666-5680
6694/SEC11C (−445); chr18:56887469-56887502/GRP (+86); chr18:56887859-56887906/GRP (+483); chr18:56
888327-56888384/GRP (+956); chr18:56888656-56888686/GRP (+1271), RAX (+51954); chr18:56935407-569
35479/RAX (+5182), GRP (+48043); chr18:56939652-56939763/RAX (+917); chr18:56940293-56940359/RAX
(+299); chr18:56940504-56940557/RAX (+94); chr18:56941252-56941301/RAX (−652); chr18:56941492-569415
30/RAX (−886); chr18:57364397-57364447/CCBE1 (+222); chr18:57567093-57567153/PMAIP1 (−69); chr18:590
01344-59001497/MC4R (−961420), CDH20 (−156214); chr18:59854194-59854260/KIAA1468 (−297); chr18:5999
2482-59992500/TNFRSF11A (−57); chr18:60105452-60105512/ZCCHC2 (−85176), TNFRSF11A (+112934); chr
18:60190048-60190084/ZCCHC2 (−592); chr18:60263544-60263660/PHLPP1 (−119070), ZCCHC2 (+72944); ch
r18:60985533-60985592/BCL2 (+1050), PHLPP1 (+602891); chr18:63418027-63418056/CDH7 (+554); chr18:6
7067971-67067995/DOK6 (−301); chr18:70209013-70209101/CBLN2 (+2666); chr18:70209437-70209473/CBL
N2 (+2268); chr18:70533794-70533988/NETO1 (+919); chr18:70534338-70534427/NETO1 (+427); chr18:7053
4965-70535184/NETO1 (−265); chr18:70535555-70535575/NETO1 (−755); chr18:70536084-70536187/NETO1
(−1326); chr18:72124190-72124237/FAM69C (+289); chr18:72265466-72265482/ZNF407 (−77449), CNDP1 (+63
782); chr18:72916860-72916977/ZADH2 (+4362), ZNF407 (+573996); chr18:73167311-73167421/C18orf62 (−2
7777); chr18:73168178-73168246/C18orf62 (−28623); chr18:73628246-73628367/C18orf62 (−488718), ZNF516
(+578839); chr18:74101932-74102065/C18orf62 (−962410), ZNF516 (+105147); chr18:74118110-74118196/C1
8orf62 (−978564), ZNF516 (+88993); chr18:74163574-74163597/ZNF516 (+43560); chr18:74535698-74535735/
ZNF236 (−399); chr18:74717116-74717217/MBP (+127607), ZNF236 (+181051); chr18:74766739-74766788/M
BP (+78010), ZNF236 (+230648); chr18:74826680-74826773/MBP (+18047), ZNF236 (+290611); chr18:749619
56-74962018/GALR1 (−21); chr18:74962171-74962209/GALR1 (+182); chr18:74962344-74962407/GALR1 (+36
8); chr18:74963546-74963720/GALR1 (+1625); chr18:75981488-75981603/SALL3 (−758729); chr18:76541636-
76541648/SALL3 (−198633); chr18:76733725-76733854/SALL3 (−6485); chr18:76734078-76734256/SALL3 (−61
08); chr18:76737116-76737231/SALL3 (−3101); chr18:76737530-76737609/SALL3 (−2705); chr18:76740224-76
740339/SALL3 (+7); chr18:76740452-76740768/SALL3 (+335); chr18:77159295-77159405/NFATC1 (−976); chr
18:77159576-77159790/NFATC1 (−643); chr18:77199340-77199436/CTDP1 (−240413), NFATC1 (+39062); chr1
8:77397602-77398025/CTDP1 (−41987), NFATC1 (+237488); chr18:77439855-77439887/CTDP1 (+70); chr18:7
7451019-77451125/KCNG2 (−172596), CTDP1 (+11271); chr18:77548055-77548140/KCNG2 (−75570), CTDP1
(+108297); chr18:77550591-77550705/KCNG2 (−73020), CTDP1 (+110847); chr18:77557891-77557912/KCNG
2 (−65766), CTDP1 (+118101); chr18:77558397-77558416/KCNG2 (−65261), CTDP1 (+118606); chr18:7771190
1-77711925/PQLC1 (−260); chr18:77724279-77724331/HSBP1L1 (−277); chr19:267139-267205/PPAP2C (+239
97), OR4F17 (+156493); chr19:383938-384078/THEG (−7995), SHC2 (+76988); chr19:405794-405930/THEG
(−29849), SHC2 (+55134); chr19:488642-488679/SHC2 (−27665), MADCAM1 (−7829); chr19:498697-498732/TPG
S1 (−8782), MADCAM1 (+2225); chr19:519003-519034/CDC34 (−12714), TPGS1 (+11522); chr19:532781-5328
48/GZMM (−11212), CDC34 (+1082); chr19:560266-560308/BSG (−12250), GZMM (+16260); chr19:590156-590
277/HCN2 (+324); chr19:590390-590605/HCN2 (+605); chr19:591018-591031/HCN2 (+1132), POLRMT (+4254
3); chr19:680532-680552/FSTL3 (+4153), PRSS57 (+14919); chr19:681322-681567/FSTL3 (+5056), PRSS57
(+14016); chr19:695363-695567/PRSS57 (−4); chr19:695687-695794/PRSS57 (−280); chr19:708540-708630/PA
LM (−368); chr19:709782-709821/PALM (+849); chr19:720847-720901/PTBP1 (−76518), PALM (+11921); chr19:
757464-757497/PTBP1 (−39911), PALM (+48528); chr19:796733-796768/PTBP1 (−641); chr19:797084-797226/
PTBP1 (−237); chr19:797934-797942/PTBP1 (+546); chr19:830584-830713/PRTN3 (−10336), AZU1 (+2818); ch
r19:853322-853343/CFD (−6332), ELANE (+1042); chr19:863839-863983/CFD (+4246), MED16 (+29307); chr1
9:913129-913161/KISS1R (−4197), R3HDM4 (+80); chr19:925222-925261/ARID3A (−795); chr19:932840-93290
0/GRIN3B (−67567), ARID3A (+6833); chr19:933054-933096/GRIN3B (−67362), ARID3A (+7038); chr19:941030-
941246/GRIN3B (−59299), ARID3A (+15101); chr19:947649-947704/GRIN3B (−52760), ARID3A (+21640); chr1
9:947870-947935/GRIN3B (−52534), ARID3A (+21866); chr19:982781-982813/GRIN3B (−17640), ARID3A (+56
760); chr19:1021093-1021103/C19orf6 (+43); chr19:1047183-1047263/HMHA1 (−19951), ABCA7 (+7121); chr1
9:1066688-1066750/HMHA1 (−455); chr19:1068371-1068542/HMHA1 (+1283), POLR2E (+26934); chr19:10743
72-1074764/HMHA1 (+7394), POLR2E (+20823); chr19:1088313-1088409/POLR2E (+7030), HMHA1 (+21187);
chr19:1104050-1104079/GPX4 (+129); chr19:1132866-1132916/GPX4 (+28955), SBNO2 (+41391); chr19:117
4237-1174258/SBNO2 (+34); chr19:1213741-1213868/STK11 (+8007), C19orf26 (+24185); chr19:1214098-121
4206/STK11 (+8354), C19orf26 (+23838); chr19:1240537-1240586/C19orf26 (−2572), ATP5D (−1187); chr19:12
41528-1241589/C19orf26 (−3569), ATP5D (−190); chr19:1241941-1241997/C19orf26 (−3979), ATP5D (+220); ch
r19:1242156-1242211/C19orf26 (−4194), ATP5D (+435); chr19:1249336-1249389/MIDN (+811); chr19:1266006-
1266059/CIRBP (−3234); chr19:1266260-1266295/CIRBP (−2989); chr19:1275417-1275573/C19orf24 (−25); chr
19:1276190-1276217/C19orf24 (+684); chr19:1288079-1288133/MUM1 (−66870), EFNA2 (+1953); chr19:12954
26-1295723/MUM1 (−59401), EFNA2 (+9422); chr19:1295834-1295873/MUM1 (−59122), EFNA2 (+9701); chr19:
1363554-1363652/NDUFS7 (−20104), MUM1 (+8627); chr19:1402394-1402510/GAMT (−900); chr19:1457047-1
457070/APC2 (+6911), C19orf25 (+22169); chr19:1466971-1467028/C19orf25 (+12228), APC2 (+16852); chr19:
1467138-1467346/C19orf25 (+11986), APC2 (+17094); chr19:1467505-1467523/C19orf25 (+11714), APC2 (+1
7366); chr19:1467710-1467840/C19orf25 (+11453), APC2 (+17627); chr19:1468012-1468203/C19orf25 (+1112
0), APC2 (+17960); chr19:1468336-1468375/C19orf25 (+10872), APC2 (+18208); chr19:1490473-1490513/RE
EP6 (−672), PCSK4 (−86); chr19:1490954-1491008/PCSK4 (−574), REEP6 (−184); chr19:1513722-1513772/ADA
MTSL5 (−559); chr19:1523893-1523906/PLK5 (−173); chr19:1524207-1524270/PLK5 (+166); chr19:1524383-15
24442/PLK5 (+340); chr19:1591452-1591499/MEX3D (−23419), MBD3 (+1176); chr19:1608825-1608910/UQCR
11 (−3385); chr19:1650685-1650721/TCF3 (−417); chr19:1726105-1726136/TCF3 (−75835), ONECUT3 (−27541);
chr19:1734453-1734497/TCF3 (−84189), ONECUT3 (−19187); chr19:1754194-1754224/ONECUT3 (+547); chr
19:1761853-1761983/ONECUT3 (+8256), ATP8B3 (+50281); chr19:1762516-1762580/ONECUT3 (+8886), ATP
8B3 (+49651); chr19:1763799-1763934/ONECUT3 (+10205), ATP8B3 (+48332); chr19:1767752-1767771/ONE
CUT3 (+14100), ATP8B3 (+44437); chr19:1776047-1776192/ONECUT3 (+22458), ATP8B3 (+36079); chr19:18
47545-1847608/REXO1 (+875); chr19:1852073-1852148/REXO1 (−3659); chr19:1855564-1855654/REXO1 (−71
57), KLF16 (+7955); chr19:1876485-1876538/KLF16 (−12948), FAM108A1 (+9006); chr19:1967240-1967300/C
SNK1G2 (+26109), BTBD2 (+48432); chr19:2051004-2051047/MKNK2 (+217); chr19:2091134-2091272/MKNK
2 (−39960), MOB3A (+5066); chr19:2094205-2094280/IZUMO4 (−2625), MOB3A (+2026); chr19:2095431-20954
75/IZUMO4 (−1415), MOB3A (+816); chr19:2095590-2095672/IZUMO4 (−1237), MOB3A (+638); chr19:2096242-
2096277/IZUMO4 (−608), MOB3A (+9); chr19:2163871-2163895/DOT1L (−265); chr19:2240021-2240148/AMH
(−9028), SF3A2 (+3269); chr19:2251465-2251600/AMH (+2420), JSRP1 (+4889); chr19:2252068-2252588/AMH
(+3215), JSRP1 (+4094); chr19:2253736-2253780/JSRP1 (+2664), AMH (+4645); chr19:2271647-2271691/AX
747191 (−18330), OAZ1 (+2149); chr19:2273018-2273069/AX747191 (−16955), OAZ1 (+3524); chr19:2282438-
2282512/AX747191 (−7524), OAZ1 (+12955); chr19:2288131-2288257/AX747191 (−1805); chr19:2289549-2289
652/AX747191 (−398); chr19:2290908-2290980/AX747191 (+945); chr19:2291204-2291262/AX747191 (+1234),
LINGO3 (+16923); chr19:2302355-2302510/LINGO3 (+5723), AX747191 (+12434); chr19:2307668-2307775/LI
NGO3 (+434); chr19:2360537-2360618/TMPRSS9 (−29206), SPPL2B (+31949); chr19:2360810-2360884/TMP
RSS9 (−28937), SPPL2B (+32218); chr19:2427390-2427536/TIMM13 (+412); chr19:2456351-2456388/LMNB2
(+588); chr19:2474663-2474702/GADD45B (−1440); chr19:2561966-2562006/GADD45B (+85863), GNG7 (+140
760); chr19:2599159-2599231/GNG7 (+103551), GADD45B (+123072); chr19:2634462-2634572/GNG7 (+6822
9), GADD45B (+158394); chr19:2720335-2720412/GNG7 (−17628), DIRAS1 (+1016); chr19:2776019-2776121/
SLC39A3 (−35996), SGTA (+7284); chr19:2782997-2783238/THOP1 (−2388), SGTA (+236); chr19:3068307-306
8362/GNA11 (−26073), AES (−5371); chr19:3094951-3095110/GNA11 (+623); chr19:3136454-3136613/GNA15
(+343); chr19:3136878-3136946/GNA15 (+721); chr19:3178636-3178758/S1PR4 (−39); chr19:3179238-317939
6/S1PR4 (+581); chr19:3179827-3180368/NCLN (−5777), S1PR4 (+1362); chr19:3285963-3285993/NFIC (−805
87), CELF5 (+61277); chr19:3359668-3359692/NFIC (−6885), CELF5 (+134979); chr19:3369713-3369824/NFIC
(+3204), C19orf77 (+110771); chr19:3444550-3444581/C19orf77 (+35974), NFIC (+78001); chr19:3478602-34
78649/C19orf77 (+1914), NFIC (+112061); chr19:3572643-3572717/HMG20B (−263); chr19:3586007-3586074/
HMG20B (+13098), TBXA2R (+20790); chr19:3605109-3605208/TBXA2R (+1672), HMG20B (+32216); chr19:3
607603-3607674/TBXA2R (−808); chr19:3608745-3608892/TBXA2R (−1988); chr19:3631667-3631701/C19orf29
(−4871); chr19:3670855-3670990/C19orf29 (−44110), PIP5K1C (+29554); chr19:3671129-3671270/C19orf29
(−44387), PIP5K1C (+29277); chr19:3671966-3672066/C19orf29 (−45203), PIP5K1C (+28461); chr19:3688096-36
88228/C19orf29 (−61349), PIP5K1C (+12315); chr19:3785836-3785864/MATK (+565); chr19:3801529-3801582/
MATK (−15141), ZFR2 (+67471); chr19:3822110-3822134/MATK (−35707), ZFR2 (+46905); chr19:3869061-386
9118/ZFR2 (−63); chr19:3950857-3950893/ITGB1BP3 (+17774), DAPK3 (+18952); chr19:3971205-3971241/DA
PK3 (−1396); chr19:4033606-4033631/PIAS4 (+25870), ZBTB7A (+33197); chr19:4108811-4108835/ZBTB7A
(−42007), MAP2K2 (+15303); chr19:4123839-4123861/MAP2K2 (+276); chr19:4182580-4182605/SIRT6 (+3); chr
19:4246869-4246926/EBI3 (+17358), TMIGD2 (+55530); chr19:4279432-4279567/TMIGD2 (+22928), EBI3 (+4
9960); chr19:4280137-4280205/TMIGD2 (+22257), EBI3 (+50631); chr19:4302370-4302488/FSD1 (−2162), TMI
GD2 (−1); chr19:4326945-4327048/STAP2 (+11944), FSD1 (+22406); chr19:4342483-4342537/STAP2 (−3569),
MPND (−1014); chr19:4457230-4457288/UBXN6 (+532); chr19:4474233-4474305/HDGFRP2 (+2014), PLIN4
(+43447); chr19:4543428-4543486/LRG1 (−3421); chr19:4543762-4543804/LRG1 (−3747); chr19:4597187-45972
71/SEMA6B (−37458), C19orf10 (+73186); chr19:4598616-4598627/SEMA6B (−38851), C19orf10 (+71793); chr
19:4636093-4636151/SEMA6B (−76351), C19orf10 (+34293); chr19:4639620-4639656/SEMA6B (−79867), C19o
rf10 (+30777); chr19:4759828-4759875/DPP9 (−35997), FEM1A (−31876); chr19:4792487-4792507/FEM1A (+7
69); chr19:4916710-4916766/KDM4B (−52386), UHRF1 (+7228); chr19:5293138-5293155/PTPRS (+47667), KD
M4B (+324023); chr19:5294142-5294320/PTPRS (+46583), KDM4B (+325107); chr19:5338851-5338900/PTPR
S (+1938), KDM4B (+369752); chr19:5492833-5492887/ZNRF4 (+37434), SAFB2 (+130078); chr19:5585894-5
586026/SAFB2 (+36978), ZNRF4 (+130534); chr19:5622666-5622693/SAFB (−366), SAFB2 (+258); chr19:5680
463-5680500/HSD11B1L (−553), C19orf70 (+429); chr19:5686452-5686523/RPL36 (−3784); chr19:5690293-569
0319/RPL36 (+34); chr19:5802059-5802154/NRTN (−21711), DUS3L (−10858); chr19:5805686-5805711/NRTN
(−18119), DUS3L (−14450); chr19:5829319-5829398/NRTN (+5541), FUT6 (+10383); chr19:5977738-5977760/R
ANBP3 (+571); chr19:6004057-6004120/RANBP3 (−25769), RFX2 (+106575); chr19:6199358-6199422/ACSBG
2 (+63680), MLLT1 (+80569); chr19:6234235-6234300/MLLT1 (+45691), ACSBG2 (+98558); chr19:6531889-65
31936/TNFSF9 (+903); chr19:6669868-6669932/TNFSF14 (+699); chr19:6697434-6697471/TNFSF14 (−26854),
C3 (+23209); chr19:6753408-6753474/TRIP10 (+13734), SH2D3A (+14082); chr19:6772929-6773034/VAV1
(+260); chr19:7660686-7660714/CAMSAP3 (−88); chr19:7795692-7795734/FCER2 (−28681), CLEC4G (+1344); c
hr19:7853471-7853543/EVI5L (−57875), CLEC4M (+25472); chr19:7853798-7853832/EVI5L (−57567), CLEC4M
(+25780); chr19:7854393-7854456/EVI5L (−56957), CLEC4M (+26390); chr19:7894895-7894988/EVI5L (−1644
0), CLEC4M (+66907); chr19:7927241-7927302/LRRC8E (−26118), EVI5L (+15890); chr19:7928186-7928218/L
RRC8E (−25188), EVI5L (+16820); chr19:7990570-7990605/CTXN1 (+463); chr19:8007957-8008005/TIMM44
(+727); chr19:8070415-8070435/ELAVL1 (+104); chr19:8115058-8115114/CCL25 (−2560); chr19:8174154-8174
232/FBN3 (+38188), CCL25 (+56547); chr19:8399364-8399386/ANGPTL4 (−29636), RPS28 (+12991); chr19:86
56851-8656878/MYO1F (−14534), ADAMTS10 (+18723); chr19:8657741-8657811/MYO1F (−15445), ADAMTS1
0 (+17812); chr19:8808638-8808678/ACTL9 (+514); chr19:9251152-9251198/ZNF317 (+119), ZNF317 (+119);
chr19:9473673-9473714/ZNF177 (−13298), ZNF559 (+39246); chr19:9517791-9517879/ZNF266 (+28399), ZNF
177 (+30843); chr19:9938334-9938365/UBL5 (−206); chr19:10011106-10011189/OLFM2 (+35922), PIN1 (+652
65); chr19:10023852-10024009/OLFM2 (+23139), PIN1 (+78048); chr19:10046402-10046485/OLFM2 (+626); c
hr19:10077277-10077381/OLFM2 (−30259), COL5A3 (+43818); chr19:10138460-10138479/RDH8 (+14545), AN
GPTL6 (+74955); chr19:10341570-10341586/S1PR2 (+370); chr19:10397993-10398074/ICAM5 (−2621), ICAM4
(+391); chr19:10406160-10406278/ICAM5 (+5564), ZGLP1 (+14014); chr19:10407030-10407044/ICAM5 (+638
2), ZGLP1 (+13196); chr19:10444571-10444632/RAVER1 (−288); chr19:10514195-10514233/CDC37 (+57); chr
19:10624540-10624739/KEAP1 (−10586), S1PR5 (+4028); chr19:10624853-10624965/KEAP1 (−10855), S1PR5
(+3759); chr19:10625667-10625744/KEAP1 (−11652), S1PR5 (+2962); chr19:10655591-10655652/ATG4D (+9
75); chr19:10765276-10765339/ILF3 (+371); chr19:10982658-10982685/CARM1 (+419); chr19:11201315-1120
1350/LDLR (+1295), SPC24 (+65151); chr19:11253444-11253491/SPC24 (+13016), LDLR (+53430); chr19:114
04716-11404771/TSPAN16 (−2080); chr19:11450155-11450197/RAB3D (+168); chr19:11492662-11492786/EP
OR (+2294), LPPR2 (+26662); chr19:11532109-11532262/RGL3 (−2168); chr19:11546874-11546927/PRKCSH
(+632); chr19:11591300-11591353/ELAVL3 (+476); chr19:11591639-11591672/ELAVL3 (+147); chr19:1161620
7-11616250/ZNF653 (+509); chr19:11750771-11750824/ZNF833P (−34015), ZNF627 (+42563); chr19:1178507
8-11785183/ZNF833P (+318); chr19:12306194-12306229/ZNF136 (+32340), ZNF44 (+99502); chr19:12405169-
12405217/ZNF44 (+521); chr19:12551424-12551485/ZNF443 (+471); chr19:12624211-12624546/ZNF709 (−28
747), ZNF490 (+97244); chr19:12777330-12777367/WDR83 (−3168), MAN2B1 (+242); chr19:12831700-128317
72/FBXW9 (−24281), TNPO2 (+1726); chr19:12832955-12832993/TNPO2 (+488); chr19:12833426-12833448/T
NPO2 (+25); chr19:12995814-12995851/DNASE2 (−3498); chr19:13056576-13056619/RAD23A (−56); chr19:13
121070-13121084/NFIX (+14493), LYL1 (+92897); chr19:13122878-13123062/NFIX (+16386), LYL1 (+91004);
chr19:13135414-13135524/NFIX (+28885), LYL1 (+78505); chr19:13207431-13207596/LYL1 (+6460), NFIX (+1
00930); chr19:13209980-13210136/LYL1 (+3916), NFIX (+103474); chr19:13210332-13210683/LYL1 (+3466),
NFIX (+103924); chr19:13213763-13213789/LYL1 (+198); chr19:13215314-13215525/LYL1 (−1446); chr19:136
17066-13617105/CACNA1A (+188); chr19:13617465-13617489/CACNA1A (−203); chr19:13885202-13885244/Z
SWIM4 (−21051), MRI1 (+9886); chr19:13950331-13950466/NANOS3 (−37664), ZSWIM4 (+44125); chr19:1395
3324-13953343/NANOS3 (−34729), ZSWIM4 (+47060); chr19:13958535-13958549/NANOS3 (−29521), ZSWIM4
(+52268); chr19:13961258-13961290/NANOS3 (−26789), ZSWIM4 (+55000); chr19:14089869-14089933/POD
NL1 (−25697), RFX1 (+27233); chr19:14097426-14097489/PODNL1 (−33254), RFX1 (+19676); chr19:14117881-
14117930/RFX1 (−772); chr19:14168207-14168214/PALM3 (+1760), IL27RA (+25949); chr19:14192300-14192
325/PALM3 (−22342), SAMD1 (+8919); chr19:14247632-14247660/ASF1B (−206); chr19:14278532-14278664/A
SF1B (−31158), LPHN1 (+38399); chr19:14317680-14317696/LPHN1 (−691); chr19:14474148-14474179/LPHN1
(−157167), CD97 (−17792); chr19:14492401-14492435/CD97 (+462); chr19:14530064-14530104/DDX39A (+11
1); chr19:14583516-14583613/PTGER1 (+2609), PKN1 (+32479); chr19:14584073-14584167/PTGER1 (+2054),
PKN1 (+33034); chr19:14584413-14584536/PTGER1 (+1699), PKN1 (+33389); chr19:14591052-14591075/PT
GER1 (−4890); chr19:14628992-14629016/DNAJB1 (+197); chr19:14886771-14886822/EMR2 (+2556), ZNF333
(+85927); chr19:15090729-15090745/CCDC105 (−30802), SLC1A6 (−7008); chr19:15127161-15127234/CASP1
4 (−33093), CCDC105 (+5659); chr19:15344131-15344417/EPHX3 (−1043); chr19:15442261-15442303/BRD4
(−51020), AKAP8 (+48330); chr19:15528952-15528973/AKAP8L (+870); chr19:15580347-15580771/RASAL3 (−5
177), PGLYRP2 (+9756); chr19:15739954-15740083/CYP4F3 (−11688), CYP4F8 (+13990); chr19:15982551-15
982652/OR10H1 (−63666), CYP4F2 (+26282); chr19:16187620-16187640/RAB8A (−34860), TPM4 (+9313); chr
19:16197083-16197202/RAB8A (−25347), TPM4 (+18826); chr19:16222445-16222493/RAB8A (−21); chr19:163
69409-16369462/KLF2 (−66215), AP1M1 (+60771); chr19:16394456-16394556/KLF2 (−41145), AP1M1 (+85841);
chr19:16435975-16435992/KLF2 (+333); chr19:16482436-16482469/KLF2 (+46802), EPS15L1 (+100309); ch
r19:16682841-16682863/SLC35E1 (+341); chr19:16683258-16683295/SLC35E1 (−84); chr19:16738793-167388
14/MED26 (+211); chr19:16940139-16940173/SIN3B (−53); chr19:16999952-17000071/F2RL3 (+186); chr19:17
007389-17007446/F2RL3 (+7592), CPAMD8 (+130207); chr19:17137359-17137395/CPAMD8 (+248); chr19:17
246401-17246457/USE1 (−79726), MYO9B (+59838); chr19:17438895-17438948/ANO8 (+6716), MRPL34 (+22
445); chr19:17439194-17439331/ANO8 (+6375), MRPL34 (+22786); chr19:17439540-17439835/ANO8 (+5950),
MRPL34 (+23211); chr19:17439959-17439993/ANO8 (+5662), MRPL34 (+23499); chr19:17501970-17501993/
PLVAP (−13845), BST2 (+14402); chr19:17502553-17502609/PLVAP (−14444), BST2 (+13803); chr19:1756649
9-17566700/TMEM221 (−7224), NXNL1 (+5125); chr19:17622330-17622393/PGLS (−70); chr19:17634138-1763
4201/GLT25D1 (−32341), PGLS (+11738); chr19:17666922-17666974/GLT25D1 (+437); chr19:17798734-1779
8754/UNC13A (+306); chr19:17858844-17858872/B3GNT3 (−47061), MAP1S (+28555); chr19:17876020-17876
076/B3GNT3 (−29871), MAP1S (+45745); chr19:17889490-17889636/B3GNT3 (−16356), MAP1S (+59260); chr1
9:17983669-17983723/SLC5A5 (+914); chr19:18060544-18060571/KCNN1 (−1553); chr19:18061575-18061639/
KCNN1 (−504); chr19:18113542-18113567/KCNN1 (+51444), IL12RB1 (+84187); chr19:18118908-18118965/K
CNN1 (+56826), IL12RB1 (+78805); chr19:18119523-18119551/KCNN1 (+57426), IL12RB1 (+78205); chr19:18
314548-18314584/RAB3A (+308); chr19:18334731-18334784/RAB3A (−19884), PDE4C (+24252); chr19:18337
332-18337346/RAB3A (−22465), PDE4C (+21671); chr19:18343810-18343879/RAB3A (−28971), PDE4C (+1516
5); chr19:18391619-18391642/JUND (+801); chr19:18402146-18402257/JUND (−9770), LSM4 (+31799); chr19:
18539804-18539933/ISYNA1 (+9242), SSBP4 (+9723); chr19:18543840-18544012/ISYNA1 (+5185), SSBP4
(+13780); chr19:18544272-18544418/ISYNA1 (+4766), SSBP4 (+14199); chr19:18544723-18544734/ISYNA1 (+4
382), SSBP4 (+14583); chr19:18544845-18544891/ISYNA1 (+4243), SSBP4 (+14722); chr19:18654616-18654
640/FKBP8 (−245); chr19:18714581-18714789/CRLF1 (+2975), C19orf60 (+15190); chr19:18715082-18715120/
CRLF1 (+2559), C19orf60 (+15606); chr19:18722358-18722386/CRLF1 (−4712), TMEM59L (−1310); chr19:1872
2489-18722521/CRLF1 (−4845), TMEM59L (−1177); chr19:18723397-18723427/TMEM59L (−270); chr19:187248
38-18724917/CRTC1 (−69547), TMEM59L (+1196); chr19:18746969-18746991/CRTC1 (−47445), TMEM59L (+2
3298); chr19:18760846-18761118/CRTC1 (−33443), TMEM59L (+37300); chr19:18770245-18770327/CRTC1
(−24139), TMEM59L (+46604); chr19:18898233-18898314/COMP (+3840), CRTC1 (+103849); chr19:18980718-1
8980882/CERS1 (+26153), UPF1 (+38056); chr19:18981254-18981331/CERS1 (+25660), UPF1 (+38549); chr1
9:18981445-18981474/CERS1 (+25493), UPF1 (+38716); chr19:19030746-19030772/COPE (−560), DDX49 (+2
75); chr19:19174421-19174575/SLC25A42 (−305); chr19:19175238-19175260/SLC25A42 (+446); chr19:192214
08-19221464/TMEM161A (+27831), SLC25A42 (+46633); chr19:19372277-19372329/HAPLN4 (+1348), NCAN
(+49521); chr19:19571724-19571750/TSSK6 (+54732), GATAD2A (+75095); chr19:19650551-19650621/CILP2
(+1512), PBX4 (+79139); chr19:19650916-19651027/CILP2 (+1898), PBX4 (+78753); chr19:19651482-196515
12/CILP2 (+2423), PBX4 (+78228); chr19:19651848-19651990/CILP2 (+2845), PBX4 (+77806); chr19:1972953
0-19729585/PBX4 (+167); chr19:19779479-19779530/ZNF101 (−158); chr19:19971773-19971821/ZNF253 (−49
17); chr19:20606299-20606342/ZNF737 (+142305), ZNF486 (+328238); chr19:20847386-20847485/ZNF626
(−3034); chr19:21580157-21580233/ZNF493 (+274); chr19:21646534-21646606/ZNF429 (−41867), ZNF493 (+66
649); chr19:21657903-21657922/ZNF429 (−30524), ZNF493 (+77992); chr19:21769375-21769579/ZNF429 (+8
1040), ZNF100 (+180953); chr19:22034422-22034446/ZNF43 (−15464), ZNF208 (+159311); chr19:22072938-2
2073021/ZNF43 (−54010), ZNF208 (+120765); chr19:22235624-22235695/ZNF257 (+394); chr19:22604824-22
604860/ZNF98 (+306); chr19:22610701-22610862/ZNF492 (−206344), ZNF98 (−5634); chr19:22611190-226112
41/ZNF492 (−205910), ZNF98 (−6068); chr19:22611467-22611555/ZNF492 (−205615), ZNF98 (−6363); chr19:22
702467-22702527/ZNF492 (−114629), ZNF98 (−97349); chr19:22800786-22800846/ZNF98 (−195668), ZNF492
(−16310); chr19:22805769-22805823/ZNF98 (−200648), ZNF492 (−11330); chr19:22806056-22806297/ZNF98 (−2
01029), ZNF492 (−10949); chr19:22966638-22966705/ZNF99 (+301); chr19:22990247-22990300/ZNF99 (−2330
1), ZNF724P (+442898); chr19:22990460-22990568/ZNF99 (−23541), ZNF724P (+442658); chr19:23185657-23
185693/ZNF99 (−218702), ZNF724P (+247497); chr19:23185843-23186034/ZNF99 (−218966), ZNF724P (+247
233); chr19:23253201-23253315/ZNF99 (−286285), ZNF724P (+179914); chr19:23253888-23254114/ZNF99 (−2
87028), ZNF724P (+179171); chr19:23258008-23258305/ZNF99 (−291184), ZNF724P (+175015); chr19:233001
54-23300232/ZNF99 (−333220), ZNF724P (+132979); chr19:23386783-23387051/ZNF99 (−419944), ZNF724P
(+46255); chr19:23432962-23433023/ZNF724P (+179); chr19:23654080-23654103/ZNF91 (−75823), ZNF675
(+215925); chr19:23946108-23946126/ZNF681 (−4424), RPSA (+301); chr19:29218551-29218616/UQCRFS1 (+4
85552); chr19:29284317-29284329/UQCRFS1 (+419813); chr19:29284583-29284915/UQCRFS1 (+419387); ch
r19:29703991-29704027/UQCRFS1 (+127); chr19:30015963-30016024/VSTM2B (−1497); chr19:30016935-300
17014/VSTM2B (−516); chr19:30017510-30017577/VSTM2B (+53); chr19:30017722-30017765/VSTM2B (+253);
chr19:30019611-30019660/POP4 (−77534), VSTM2B (+2145); chr19:30019931-30019993/POP4 (−77208), VS
TM2B (+2471); chr19:30021039-30021252/POP4 (−76024), VSTM2B (+3655); chr19:30205890-30205915/C19o
rf12 (+60); chr19:30363476-30363523/URI1 (−69646), CCNE1 (+60599); chr19:30713593-30713685/ZNF536
(−149689), URI1 (+280493); chr19:30716770-30716952/ZNF536 (−146467), URI1 (+283715); chr19:30866272-30
866452/ZNF536 (+3034), TSHZ3 (+973828); chr19:31839644-31839675/TSHZ3 (+530); chr19:31839928-31839
941/TSHZ3 (+255); chr19:31842489-31842530/TSHZ3 (−2320); chr19:31842672-31842828/TSHZ3 (−2560); chr
19:31842964-31843033/TSHZ3 (−2809); chr19:31844884-31844953/TSHZ3 (−4729); chr19:31845488-31845580/
ZNF507 (−990980), TSHZ3 (−5344); chr19:32715258-32715442/TSHZ3 (−875160), ZNF507 (−121164); chr19:33
165568-33165621/RGS9BP (−718), ANKRD27 (+507); chr19:33166969-33166988/ANKRD27 (−877), RGS9BP
(+666); chr19:33167488-33167496/ANKRD27 (−1390), RGS9BP (+1179); chr19:33462717-33462754/C19orf40
(−412), CEP89 (+133); chr19:33625776-33625803/LRP3 (−59809), GPATCH1 (+54004); chr19:33717909-337179
38/SLC7A10 (−1168); chr19:33740342-33740428/SLC7A10 (−23629), CEBPA (+53045); chr19:33794745-33794
779/CEBPA (−1332); chr19:34112287-34112326/CHST8 (−554); chr19:34113303-34113366/CHST8 (+474); chr1
9:34114050-34114286/KCTD15 (−173583), CHST8 (+1307); chr19:34288989-34289043/LSM14A (−374336), KC
TD15 (+1265); chr19:34311907-34311954/LSM14A (−351421), KCTD15 (+24180); chr19:34745977-34745998/
GPI (−110044), LSM14A (+82636); chr19:34972006-34972067/WTIP (−843); chr19:34972364-34972522/WTIP
(−437); chr19:34972648-34972675/WTIP (−218); chr19:34973124-34973242/WTIP (+303); chr19:34973457-3497
3553/WTIP (+625); chr19:34991198-34991282/WTIP (+18360), SCGB2B2 (+94250); chr19:34991497-3499158
0/WTIP (+18659), SCGB2B2 (+93951); chr19:35068424-35068601/SCGB2B2 (+16977), WTIP (+95633); chr19:
35395949-35396250/ZNF599 (−131966), ZNF30 (−21707); chr19:35600660-35600733/FXYD3 (−6035), HPN (+6
9287); chr19:35634335-35634415/FXYD7 (+221); chr19:36049368-36049396/ATP4A (+5178), TMEM147 (+128
80); chr19:36134526-36134593/COX6B1 (−4565); chr19:36208105-36208151/MLL4 (−793); chr19:36208350-36
208402/MLL4 (−545); chr19:36208605-36208640/MLL4 (−298); chr19:36231248-36231295/IGFLR1 (+2248), ML
L4 (+22351); chr19:36231513-36231553/PSENEN (−4961), IGFLR1 (+1987); chr19:36247183-36247230/HSPB
6 (+723); chr19:36247540-36247595/HSPB6 (+362); chr19:36247848-36247941/HSPB6 (+35); chr19:36319708-
36319779/PRODH2 (−15543), NPHS1 (+23151); chr19:36351456-36351528/APLP1 (−7909), KIRREL2 (+3682);
chr19:36390629-36390661/HCST (−2737), NFKBID (+907); chr19:36450296-36450544/SDHAF1 (−35670), LRF
N3 (+22398); chr19:36544819-36544859/WDR62 (−944), THAP8 (+825); chr19:36545415-36545479/WDR62 (−3
36), THAP8 (+217); chr19:36618842-36618901/POLR2I (−12666), CAPNS1 (−12046); chr19:36631054-3663112
1/CAPNS1 (+170); chr19:36705492-36705546/ZNF146 (+15), ZNF565 (+47); chr19:36736296-36736314/ZNF5
65 (−30739), ZFP14 (+133800); chr19:36736599-36736765/ZNF565 (−31116), ZFP14 (+133423); chr19:367484
83-36748502/ZNF565 (−42927), ZFP14 (+121612); chr19:36822467-36822557/ZNF565 (−116946), ZFP14 (+47
593); chr19:36909349-36909623/ZFP82 (+64); chr19:36912350-36912480/ZFP82 (−2865); chr19:37095813-370
96219/ZNF382 (−205); chr19:37157196-37157284/ZNF461 (+499); chr19:37288425-37288606/ZNF850 (−24800),
ZNF790 (+53173); chr19:37329389-37329447/ZNF850 (−65702), ZNF790 (+12271); chr19:37341102-373411
65/ZNF345 (−644), ZNF790 (+555); chr19:37464568-37464666/ZNF420 (−104765), ZNF568 (+57386); chr19:37
498730-37498799/ZNF420 (−70617), ZNF568 (+91534); chr19:37742793-37742848/HKR1 (−82759), ZNF383
(+25455); chr19:37861691-37861741/ZNF527 (−343); chr19:37959801-37959873/ZNF569 (−1498), ZNF570 (−145);
chr19:38042137-38042352/ZNF540 (−28); chr19:38182960-38183111/ZNF781 (+180); chr19:38210158-38210
326/ZNF607 (+449); chr19:38307920-38307977/SIPA1L3 (−89919), ZNF573 (−37719); chr19:38308337-383083
94/SIPA1L3 (−89502), ZNF573 (−38136); chr19:38346077-38346133/ZNF573 (−75875), SIPA1L3 (−51763); chr1
9:38700454-38700504/DPF1 (+14411), SIPA1L3 (+302611); chr19:38747078-38747106/PPP1R14A (+139); chr
19:38806608-38806627/KCNK6 (−3866), YIF1B (−12); chr19:38877455-38877688/SPRED3 (−3367), GGN (+109
6); chr19:38885341-38885383/SPRED3 (+4423), RASGRP4 (+31583); chr19:38886153-38886348/SPRED3 (+5
312), RASGRP4 (+30694); chr19:38886518-38886739/SPRED3 (+5690), RASGRP4 (+30316); chr19:39056352-
39056367/MAP4K1 (+52283), RYR1 (+132020); chr19:39228135-39228177/CAPN12 (+6958), ACTN4 (+89889);
chr19:39260649-39260751/CAPN12 (−25586), LGALS7 (+3457); chr19:39260917-39260984/CAPN12 (−25837),
LGALS7 (+3206); chr19:39261270-39261342/CAPN12 (−26192), LGALS7 (+2851); chr19:39261605-3926171
7/CAPN12 (−26547), LGALS7 (+2496); chr19:39282654-39282726/LGALS7B (+2840), LGALS4 (+21050); chr19:
39283038-39283060/LGALS7B (+3199), LGALS4 (+20691); chr19:39283577-39283611/LGALS7B (+3744), LG
ALS4 (+20146); chr19:39342695-39342707/HNRNPL (−2084); chr19:39360177-39360270/HNRNPL (−19607), R
INL (+8695); chr19:39360489-39360564/HNRNPL (−19910), RINL (+8392); chr19:39390038-39390193/NFKBIB
(−454), SIRT2 (+386); chr19:39574805-39574877/PAPL (−104); chr19:39589237-39589265/PAK4 (−27169), PAP
L (+14306); chr19:39589561-39589679/PAK4 (−26800), PAPL (+14675); chr19:39687647-39687755/NCCRP1
(+97); chr19:39738310-39738521/IL28B (−2807); chr19:39755233-39755264/IL28A (−3908); chr19:39755665-39
755753/IL28A (−3448); chr19:39756043-39756095/IL28A (−3088); chr19:39826508-39826667/GMFG (+138); chr
19:39898366-39898405/ZFP36 (+899); chr19:39926841-39926903/RPS16 (−254); chr19:39936031-39936090/S
UPT5H (−125); chr19:39993657-39993711/SELV (−12069), DLL3 (+4127); chr19:39997733-39997748/SELV (−8
012), DLL3 (+8184); chr19:39998254-39998331/SELV (−7460), DLL3 (+8736); chr19:40006161-40006186/SELV
(+421); chr19:40006448-40006498/SELV (+720); chr19:40502617-40502695/ZNF546 (−287); chr19:40596645-
40596689/ZNF780A (+178); chr19:40732304-40732335/CNTD2 (+277); chr19:40780593-40780656/CNTD2 (−4
8028), AKT2 (+10677); chr19:40854189-40854239/PLD3 (−118); chr19:40854803-40854836/PLD3 (+488); chr1
9:40904669-40904701/HIPK4 (−8591), PRX (+14586); chr19:41019063-41019075/SHKBP1 (−63688), SPTBN4
(+45943); chr19:41060385-41060498/SHKBP1 (−22315), SPTBN4 (+87316); chr19:41073858-41074018/SHKBP
1 (−8819), SPTBN4 (+100812); chr19:41076342-41076403/SHKBP1 (−6384), SPTBN4 (+103247); chr19:411030
38-41103133/LTBP4 (−55); chr19:41116417-41116513/LTBP4 (+13324), NUMBL (+80091); chr19:41119277-41
119370/LTBP4 (+16183), NUMBL (+77232); chr19:41119802-41120262/LTBP4 (+16891), NUMBL (+76524); ch
r19:41283765-41283840/RAB4B (−321); chr19:41316613-41316675/EGLN2 (+11310), CYP2A6 (+39708); chr19:
41317184-41317260/EGLN2 (+11888), CYP2A6 (+39130); chr19:41354455-41354575/CYP2A6 (+1837), EGL
N2 (+49181); chr19:41596042-41596078/CYP2F1 (−24293), CYP2A13 (+1692); chr19:41641363-41641382/CY
P2S1 (−57742), CYP2F1 (+21020); chr19:41641980-41642001/CYP2S1 (−57124), CYP2F1 (+21638); chr19:421
50687-42150720/CEACAM4 (−17262), CEACAM7 (+41392); chr19:42364210-42364261/RPS19 (+248); chr19:4
2426880-42427015/RABAC1 (+36580), ARHGEF1 (+38502); chr19:42432047-42432081/RABAC1 (+31464), A
RHGEF1 (+43618); chr19:42445124-42445145/RABAC1 (+18393), ARHGEF1 (+56689); chr19:42502730-4250
2763/ATP1A3 (−4365); chr19:42502938-42502971/ATP1A3 (−4573); chr19:42503259-42503323/ATP1A3 (−4909);
chr19:42573146-42573251/GRIK5 (−3242); chr19:42681161-42681174/POU2F2 (−44543), DEDD2 (+40645);
chr19:42703550-42703641/POU2F2 (−66971), DEDD2 (+18217); chr19:42759335-42759445/ERF (−81); chr19:4
2772451-42772478/CIC (−16352), ERF (−13156); chr19:42772908-42772963/CIC (−15881), ERF (−13627); chr19:
42785219-42785283/CIC (−3566); chr19:42827820-42827981/MEGF8 (−1860); chr19:42828085-42828316/ME
GF8 (−1560); chr19:44084626-44084701/XRCC1 (−4934), LOC390940 (+3712); chr19:44258703-44258747/SM
G9 (+417); chr19:44331152-44331205/ZNF283 (−294); chr19:44905714-44905766/ZFP112 (−44884), ZNF229
(+46925); chr19:45256824-45256856/CBLC (−24286), BCL3 (+4862); chr19:45351049-45351096/TOMM40 (−434
04), PVRL2 (+1680); chr19:45394085-45394133/TOMM40 (−368); chr19:45447143-45447219/CLPTM1 (−11418),
APOC1 (+29260); chr19:45596303-45596354/GEMIN7 (+13811), NKPD1 (+67079); chr19:45608019-456080
68/GEMIN7 (+25526), NKPD1 (+55364); chr19:45655349-45655399/NKPD1 (+8034), GEMIN7 (+72856); chr19:
45655557-45655647/NKPD1 (+7806), GEMIN7 (+73084); chr19:45656743-45656790/NKPD1 (+6641), GEMIN7
(+74249); chr19:45662231-45662397/NKPD1 (+1094), GEMIN7 (+79796); chr19:45681470-45681509/BLOC1
S3 (−513), TRAPPC6A (−5); chr19:45681956-45681986/TRAPPC6A (−486), BLOC1S3 (−32); chr19:45719075-45
719136/EXOC3L2 (+18363), BLOC1S3 (+37103); chr19:45752201-45752240/MARK4 (−2295), MARK4 (−2295);
chr19:45754356-45754373/MARK4 (−151), MARK4 (−151); chr19:45889225-45889315/ERCC2 (−15425), PPP1R
13L (+19042); chr19:45899742-45899782/ERCC2 (−25917), PPP1R13L (+8550); chr19:45899960-45899995/E
RCC2 (−26133), PPP1R13L (+8334); chr19:45901452-45901492/ERCC2 (−27627), PPP1R13L (+6840); chr19:4
5976065-45976122/FOSB (+4841), RTN2 (+24219); chr19:45996371-45996469/RTN2 (+3893), FOSB (+25167);
chr19:45999767-45999789/PPM1N (−1953), RTN2 (+535); chr19:46000092-46000121/PPM1N (−1624), RTN2
(+206); chr19:46002353-46002431/RTN2 (−2079), PPM1N (+661); chr19:46088063-46088101/OPA3 (+40); chr1
9:46105355-46105395/GPR4 (+91); chr19:46145506-46145557/GPR4 (−40066), EML2 (+3243); chr19:4617187
1-46171944/GIPR (+406); chr19:46184807-46184845/SNRPD2 (+10617), GIPR (+13324); chr19:46195058-461
95120/QPCTL (−652), SNRPD2 (+354); chr19:46195512-46195550/QPCTL (−210), SNRPD2 (−88); chr19:46282
589-46282650/SIX5 (−10123), DMPK (+3195); chr19:46283079-46283125/SIX5 (−10605), DMPK (+2713); chr19:
46312722-46312846/DMWD (−16724), RSPH6A (+5821); chr19:46318386-4631 8438/RSPH6A (+193); chr19:4
6318900-46318943/RSPH6A (−317); chr19:46319071-46319162/RSPH6A (−512); chr19:46319312-46319377/R
SPH6A (−740); chr19:46366230-46366249/FOXA3 (−1278), SYMPK (+308); chr19:46379858-46379893/IRF2BP
1 (+9500), FOXA3 (+12358); chr19:46380427-46380471/IRF2BP1 (+8927), FOXA3 (+12931); chr19:46390078-
46390100/IRF2BP1 (−713); chr19:46405983-46406033/MYPOP (−146); chr19:46477060-46477118/NOVA2 (−43
2); chr19:46526046-46526090/PGLYRP1 (+488); chr19:46526198-46526289/PGLYRP1 (+312); chr19:4677440
7-46774485/HIF3A (−25857), IGFL1 (+41437); chr19:46800317-46800353/HIF3A (+32); chr19:46916988-46917
060/CCDC8 (−105); chr19:46974609-46974831/CCDC8 (−57801), LOC100506012 (+129737); chr19:46993261-
46993281/CCDC8 (−76352), LOC100506012 (+111186); chr19:46996515-46996577/CCDC8 (−79627), LOC100
506012 (+107911); chr19:46996848-46996886/CCDC8 (−79948), LOC100506012 (+107590); chr19:46997147-4
6997221/CCDC8 (−80265), LOC100506012 (+107273); chr19:46997506-46997786/CCDC8 (−80727), LOC1005
06012 (+106811); chr19:47152024-47152061/GNG8 (−14104), PRKD2 (+68341); chr19:47152447-47152977/G
NG8 (−14773), PRKD2 (+67672); chr19:47164200-47164236/GNG8 (−26279), PRKD2 (+56166); chr19:4726001
2-47260071/STRN4 (−10322), SLC1A5 (+31800); chr19:47358909-47358941/AP2S1 (−4722); chr19:47363403-4
7363474/ARHGAP35 (−58494), AP2S1 (−9236); chr19:47507442-47507572/NPAS1 (−16636), ARHGAP35 (+855
74); chr19:47615994-47616025/ZC3H4 (+999); chr19:47729824-47729850/BBC3 (+6186), SAE1 (+95757); chr
19:47733984-47734047/BBC3 (+2007), SAE1 (+99936); chr19:47734187-47734361/BBC3 (+1749), SAE1 (+10
0194); chr19:47742557-47742701/PRR24 (−35513), BBC3 (−6606); chr19:47778625-47778738/PRR24 (+540); c
hr19:47910584-47910679/MEIS3 (+12153), DHX34 (+58094); chr19:47950313-47950337/MEIS3 (−27540), SLC
8A2 (+24982); chr19:47960586-47960799/MEIS3 (−37908), SLC8A2 (+14614); chr19:47987585-47987618/KPT
N (−81); chr19:48018184-48018253/NAPA (+296); chr19:48048438-48048617/NAPA (−30013), ZNF541 (+10585);
chr19:48048750-48048810/NAPA (−30265), ZNF541 (+10333); chr19:48111580-48111632/GLTSCR1 (+153);
chr19:48113644-48113681/EHD2 (−102938), GLTSCR1 (+2210); chr19:48233417-48233533/GLTSCR2 (−15318),
EHD2 (+16874); chr19:48565268-48565308/CABP5 (−17977), PLA2G4C (+48821); chr19:48625550-4862567
2/PLA2G4C (−11502), LIG1 (+47949); chr19:48866703-48866749/SYNGR4 (−925), TMEM143 (+460); chr19:488
67292-48867354/SYNGR4 (−328), TMEM143 (−137); chr19:48919031-48919305/GRWD1 (−29862), GRIN2D (+2
1036); chr19:48946056-48947271/GRWD1 (−2366); chr19:48947478-48947634/GRWD1 (−1474); chr19:489477
76-48947874/GRWD1 (−1205); chr19:48983734-48983933/CYTH2 (+11369), LMTK3 (+32612); chr19:48984139-
48984222/CYTH2 (+11716), LMTK3 (+32265); chr19:49217489-49217526/MAMSTR (+5468), FUT2 (+18280);
chr19:49223903-49223950/MAMSTR (−951); chr19:49242374-49242393/MAMSTR (−19408), RASIP1 (+1586); c
hr19:49242808-49242911/MAMSTR (−19884), RASIP1 (+1110); chr19:49243237-49243285/RASIP1 (+709); chr
19:49250408-49250538/IZUMO1 (−307); chr19:49263884-49263907/FGF21 (+4748), BCAT2 (+50424); chr19:4
9339610-49339737/HSD17B14 (+260); chr19:49340491-49340573/HSD17B14 (−598); chr19:49340710-493407
52/HSD17B14 (−797); chr19:49469254-49469315/FTL (+719); chr19:49560015-49560066/CGB8 (−7673), NTF4
(+7078); chr19:49575539-49575596/KCNA7 (+630); chr19:49575744-49575764/KCNA7 (+444); chr19:4957609
3-49576122/KCNA7 (+90); chr19:49622965-49623031/PPFIA3 (+335); chr19:49623140-49623172/PPFIA3 (+49
3); chr19:49646116-49646245/HRC (+12500), PPFIA3 (+23518); chr19:49649245-49649464/HRC (+9326), PP
FIA3 (+26692); chr19:49867327-49867385/TEAD2 (−1642), DKKL1 (+314); chr19:49931772-49931792/PTH2
(−5084), SLC17A7 (+13026); chr19:49931951-49932009/PTH2 (−5282), SLC17A7 (+12828); chr19:49935544-499
35586/PTH2 (−8867), SLC17A7 (+9243); chr19:49936785-49936827/PTH2 (−10108), SLC17A7 (+8002); chr19:4
9945536-49945605/SLC17A7 (−763); chr19:49946528-49946538/SLC17A7 (−1725); chr19:50036568-50036596/
RCN3 (+5707), NOSIP (+47221); chr19:50037155-50037219/RCN3 (+6312), NOSIP (+46616); chr19:50037655-
50037688/RCN3 (+6797), NOSIP (+46131); chr19:50058165-50058236/NOSIP (+25602), RCN3 (+27326); chr
19:50193234-50193245/CPT1C (−1125); chr19:50312767-50312888/FUZ (+3739), AP2A1 (+42648); chr19:503
53970-50354016/PTOV1 (−423); chr19:50373419-50373487/PNKP (−2631); chr19:50393408-50393536/IL4I1
(+6675), TBC1D17 (+12790); chr19:50432390-50432411/ATF5 (+1), NUP62 (+398); chr19:50432694-50432746/N
UP62 (+79), ATF5 (+320); chr19:50651248-50651307/IZUMO2 (+15260), ZNF473 (+122066); chr19:50666291-
50666298/IZUMO2 (+243); chr19:50707082-50707093/MYH14 (+203); chr19:50833233-50833282/KCNC3 (−62
4); chr19:50861814-50861946/NAPSB (−13875), NAPSA (+7051); chr19:50931514-50931536/MYBPC2 (−4635);
chr19:51014339-51014427/JOSD2 (−38); chr19:51069359-51069402/ASPDH (−52239), LRRC4B (+1921); chr1
9:51071637-51071706/LRRC4B (−370); chr19:51109404-51109485/LRRC4B (−38143), SYT3 (+33647); chr19:5
1142579-51142587/SYT3 (+509); chr19:51142758-51142834/SYT3 (+296); chr19:51162254-51162333/SYT3
(−19202), SHANK1 (+57901); chr19:51169771-51169933/SYT3 (−26760), SHANK1 (+50343); chr19:51170295-51
170382/SYT3 (−27247), SHANK1 (+49856); chr19:51170738-51170905/SYT3 (−27730), SHANK1 (+49373); chr
19:51171276-51171642/SYT3 (−28367), SHANK1 (+48736); chr19:51171861-51171949/SYT3 (−28813), SHANK
1 (+48290); chr19:51189966-51190018/SYT3 (−46900), SHANK1 (+30203); chr19:51198124-51198175/SYT3
(−55058), SHANK1 (+22045); chr19:51228269-51228368/GPR32 (−45539), CLEC11A (+1714); chr19:51228639-5
1228702/GPR32 (−45187), CLEC11A (+2066); chr19:51307186-51307242/ACPT (+13542), KLK1 (+19829); chr
19:51307554-51307566/ACPT (+13888), KLK1 (+19483); chr19:51569547-51569691/KLK13 (−1252); chr19:516
70785-51670859/CD33 (−57513), SIGLEC7 (+25264); chr19:51714343-51714438/CD33 (−13944), SIGLEC7 (+6
8833); chr19:51831003-51831120/VSIG10L (+14316), IGLON5 (+15960); chr19:51924565-51924703/SIGLEC1
0 (−3577); chr19:51966521-51966591/SIGLEC8 (−4848); chr19:52097948-52097998/FLJ30403 (−340); chr19:52
104579-52104609/FLJ30403 (−6961), SIGLEC5 (+29133); chr19:52104728-52104759/FLJ30403 (−7111), SIGL
EC5 (+28983); chr19:52206607-52206801/SIGLEC14 (−56572), HAS1 (+20517); chr19:52222938-52223075/SI
GLEC14 (−72875), HAS1 (+4214); chr19:52452336-52452494/ZNF613 (+21727), ZNF350 (+37664); chr19:5248
9213-52489320/ZNF350 (+812); chr19:52489710-52489782/ZNF350 (+333); chr19:52773079-52773134/ZNF76
6 (+283); chr19:52839718-52839741/ZNF610 (+232); chr19:52873534-52873570/ZNF880 (+382); chr19:52901
116-52901156/ZNF528 (+15); chr19:52954612-52954625/ZNF578 (−2210); chr19:52954739-52954786/ZNF578
(−2066); chr19:52957112-52957230/ZNF578 (+342); chr19:52996162-52996250/ZNF808 (−34703), ZNF578 (+3
9377); chr19:52996474-52996553/ZNF808 (−34395), ZNF578 (+39685); chr19:53030735-53030790/ZNF808 (−1
46); chr19:53031571-53031592/ZNF808 (+673); chr19:53039077-53039202/ZNF701 (−34777), ZNF808 (+8231);
chr19:53039903-53039918/ZNF701 (−34006), ZNF808 (+9002); chr19:53073241-53073313/ZNF701 (−640); ch
r19:53073773-53073819/ZNF701 (−121); chr19:53141619-53141647/ZNF83 (+11); chr19:53324277-53324326/
ZNF28 (+620); chr19:53496787-53496813/ZNF816 (−30636), AK127846 (−13965); chr19:53540895-53541103/E
RVV-2 (−6992), ERVV-1 (+23655); chr19:53635764-53635784/ZNF415 (+399); chr19:53832402-53832437/ZNF
845 (−4582); chr19:53836912-53836953/ZNF845 (−69); chr19:53837218-53837281/ZNF845 (+248); chr19:5386
9350-53869376/ZNF525 (+395); chr19:53966539-53966563/ZNF813 (−4438); chr19:53966724-53966788/ZNF8
13 (−4233); chr19:53971040-53971109/ZNF813 (+86); chr19:54372624-54372682/PRKCG (−12814), MYADM
(+3042); chr19:54393162-54393191/CACNG7 (−22814), PRKCG (+7710); chr19:54412749-54412808/CACNG7
(−3212); chr19:54412992-54413008/CACNG7 (−2991); chr19:54445297-54445558/CACNG8 (−20862), CACNG7
(+29437); chr19:54481438-54481540/CACNG6 (−14053), CACNG8 (+15199); chr19:54483306-54483364/CACN
G6 (−12207), CACNG8 (+17045); chr19:54485647-54485672/CACNG6 (−9882), CACNG8 (+19370); chr19:5448
5997-54486106/CACNG6 (−9490), CACNG8 (+19762); chr19:54600172-54600225/TARM1 (−15565), OSCAR
(+4043); chr19:54606665-54606697/OSCAR (−2439), OSCAR (−2439), NDUFA3 (+521); chr19:54640384-546404
52/CNOT3 (−1018); chr19:54641467-54641603/CNOT3 (+99); chr19:54890155-54890204/TTYH1 (−36425), LAI
R1 (−13459); chr19:54895995-54896056/TTYH1 (−30579), LAIR1 (−19305); chr19:54900316-54900370/TTYH1
(−26262), LAIR1 (−23622); chr19:54926492-54926520/TTYH1 (−99); chr19:54929363-54929429/LENG8 (−30669),
TTYH1 (+2791); chr19:54941761-54941850/LENG8 (−18259), TTYH1 (+15201); chr19:54976257-54976536/LE
NG9 (−1503); chr19:55393048-55393098/NCR1 (−24435), FCAR (+7524); chr19:55592184-55592212/EPS8L1
(+4977), PPP1R12C (+36729); chr19:55592834-55592898/EPS8L1 (+5645), PPP1R12C (+36061); chr19:55593
254-55593613/EPS8L1 (+6213), PPP1R12C (+35493); chr19:55593850-55593920/EPS8L1 (+6664), PPP1R12
C (+35042); chr19:55597837-55597878/EPS8L1 (+10637), PPP1R12C (+31069); chr19:55598064-55598175/E
PS8L1 (+10899), PPP1R12C (+30807); chr19:55598375-55598488/EPS8L1 (+11211), PPP1R12C (+30495); ch
r19:55598702-55598788/EPS8L1 (+11524), PPP1R12C (+30182); chr19:55652596-55652653/PPP1R12C (−23
698), TNNT1 (+7981); chr19:55672425-55672457/TNNI3 (−3484); chr19:55691819-55691863/SYT5 (−121); chr1
9:55765916-55766061/TMEM86B (−25357), PPP6R1 (+4049); chr19:55766974-55767069/TMEM86B (−26390),
PPP6R1 (+3016); chr19:55770353-55770669/PPP6R1 (−473); chr19:55791407-55791444/BRSK1 (−4108), HSP
BP1 (+325); chr19:55895939-55896038/RPL28 (−1311), TMEM238 (−362); chr19:55897689-55897721/TMEM23
8 (−2078), RPL28 (+405); chr19:55944812-55945054/UBE2S (−25608), SHISA7 (+9297); chr19:55962940-5596
3044/SHISA7 (−8762), ISOC2 (+10057); chr19:55963980-55964040/SHISA7 (−9780), ISOC2 (+9039); chr19:559
64202-55964240/SHISA7 (−9991), ISOC2 (+8828); chr19:56028633-56028662/SBK2 (+19787), SSC5D (+2877
8); chr19:56116897-56116931/ZNF865 (−8045), ZNF524 (+5184); chr19:56186358-56186400/EPN1 (−1612); chr
19:56728603-56728658/ZSCAN5B (−24210), ZSCAN5A (+11028); chr19:56728789-56729207/ZSCAN5B (−2457
7), ZSCAN5A (+10661); chr19:56879394-56879474/ZNF542 (−56); chr19:56904704-56904723/ZNF582 (+175);
chr19:56988284-56988383/ZNF667 (+436); chr19:56988727-56988944/ZNF667 (−66); chr19:56989101-569891
73/ZNF667 (−367); chr19:56989352-56989501/ZNF667 (−657); chr19:56989626-56989696/ZNF667 (−891); chr1
9:57049785-57049807/ZFP28 (−521); chr19:57049987-57050064/ZFP28 (−291); chr19:57050288-57050311/ZF
P28 (−17); chr19:57079299-57079380/ZNF470 (+450); chr19:57106551-57106588/ZNF71 (−94); chr19:5717224
6-57172275/ZNF835 (+10862), ZNF71 (+65597); chr19:57183127-57183373/ZNF835 (−127); chr19:57349203-5
7349240/ZNF835 (−166099), PEG3 (+2872); chr19:57610828-57610895/PEG3 (−258768), USP29 (−20647); chr
19:57617716-57617831/PEG3 (−265680), USP29 (−13735); chr19:57618170-57618408/PEG3 (−266195), USP2
9 (−13220); chr19:57683163-57683239/DUXA (−4345); chr19:57683686-57683805/DUXA (−4890); chr19:577517
00-57751722/ZNF805 (−342); chr19:57831987-57832024/ZNF543 (+141); chr19:57862958-57863221/ZNF304
(+445); chr19:57989048-57989098/ZNF772 (−135); chr19:58071676-58071708/ZNF550 (−3967); chr19:5809489
1-58094924/ZNF416 (−4665), ZIK1 (−720); chr19:58220393-58220515/ZNF154 (+125); chr19:58238746-582389
27/ZNF671 (+158); chr19:58446341-58446423/ZNF418 (+358); chr19:58458891-58458978/ZNF256 (+142); chr
19:58513464-58513525/C19orf18 (−27593), ZNF606 (+1219); chr19:58521316-58521378/ZSCAN1 (−24087), ZN
F606 (−6633); chr19:58545388-58545577/ZSCAN1 (+49); chr19:58570431-58570622/ZNF135 (−80); chr19:5857
0770-58570949/ZNF135 (+253); chr19:58609360-58609472/ZSCAN18 (+20377), ZNF135 (+38809); chr19:586
09601-58609681/ZSCAN18 (+20152), ZNF135 (+39034); chr19:58629407-58629750/ZSCAN18 (+214); chr19:5
8629864-58629974/ZSCAN18 (−126); chr19:58666495-58666550/ZNF329 (−4375); chr19:58838207-58838257/
ZSCAN22 (−153); chr19:58858498-58858560/A1BG (+6336), ZSCAN22 (+20144); chr19:58858805-58858901/A
1BG (+6012), ZSCAN22 (+20468); chr19:58867619-58868196/A1BG (−3043); chr19:58873410-58873523/ZNF4
97 (+747); chr19:58907668-58907722/ZNF584 (−12368), RPS5 (+9059); chr19:58907982-58908310/ZNF584 (−1
1917), RPS5 (+9510); chr19:58919438-58919475/ZNF584 (−606); chr19:58951401-58951525/ZNF132 (+126); c
hr19:58952080-58952140/ZNF132 (−521); chr19:59025258-59025344/SLC27A5 (−1869); chr19:59030911-5903
0947/ZBTB45 (−8); chr2:45932-45969/FAM110C (+637); chr2:467942-468115/FAM150B (−179721), TMEM18
(+209410); chr2:468242-468414/FAM150B (−180020), TMEM18 (+209111); chr2:636827-636940/FAM150B (−348
576), TMEM18 (+40555); chr2:946218-946235/SNTG2 (−327); chr2:946404-946432/SNTG2 (−136); chr2:94665
7-946761/SNTG2 (+155); chr2:947044-947126/SNTG2 (+531); chr2:1253662-1253808/TPO (−163498), SNTG2
(+307181); chr2:1609016-1609081/PXDN (+139242), TPO (+191816); chr2:1747041-1747112/PXDN (+1214),
TPO (+329844); chr2:1748008-1748353/PXDN (+110); chr2:3750859-3751579/ALLC (+45433); chr2:5831205-5
831237/SOX11 (−1578); chr2:5831401-5831467/SOX11 (−1365); chr2:5832048-5832129/SOX11 (−710); chr2:58
32429-5832531/SOX11 (−319); chr2:5833080-5833131/SOX11 (+307); chr2:5833433-5833499/SOX11 (+667);
chr2:5833640-5833734/SOX11 (+888); chr2:5836575-5836621/SOX11 (+3799); chr2:5836745-5836827/SOX11
(+3987); chr2:5837073-5837307/SOX11 (+4391); chr2:5837468-5837518/SOX11 (+4694); chr2:6121566-6121
679/SOX11 (+288824), CMPK2 (+884313); chr2:7148361-7148419/RNF144A (+90867); chr2:7148594-7148645/
RNF144A (+91097); chr2:7571123-7571157/RNF144A (+513617); chr2:8597390-8597431/ID2 (−224702); chr2:
8685238-8685302/ID2 (−136843); chr2:8715636-8715708/ID2 (−106441); chr2:8715980-8716060/ID2 (−106093);
chr2:8723992-8724059/ID2 (−98087); chr2:8816823-8816862/ID2 (−5270); chr2:9235784-9235971/ASAP2 (−11
1016), MBOAT2 (−92002); chr2:9695818-9695862/ADAM17 (+77); chr2:10091589-10091720/GRHL1 (−137); chr
2:10092048-10092071/GRHL1 (+268); chr2:10183124-10183213/KLF11 (−513); chr2:10183462-10183485/KLF
11 (−208); chr2:10183803-10183814/KLF11 (+127); chr2:10220941-10220958/CYS1 (−412); chr2:10231736-102
31886/RRM2 (−30884), CYS1 (−11273); chr2:10444130-10444193/HPCAL1 (+1122), ODC1 (+144291); chr2:10
471419-10471620/HPCAL1 (+28480), ODC1 (+116933); chr2:10550210-10550363/ODC1 (+38166), HPCAL1
(+107247); chr2:10572412-10572451/ODC1 (+16021), HPCAL1 (+129392); chr2:10688931-10689017/ODC1 (−1
00521), NOL10 (+141138); chr2:11052693-11052752/KCNF1 (+660); chr2:11167847-11167913/PQLC3 (−1276
60), KCNF1 (+115817); chr2:11621923-11622000/GREB1 (−52280), E2F6 (−15665); chr2:11622166-11622259/
GREB1 (−52029), E2F6 (−15916); chr2:11622402-11622569/GREB1 (−51756), E2F6 (−16189); chr2:11810116-1
1810146/NTSR2 (+198); chr2:11817712-11817794/LPIN1 (−68987), NTSR2 (−7424); chr2:11837897-11838012/
LPIN1 (−48785), NTSR2 (−27626); chr2:12197763-12197875/TRIB2 (−659179), LPIN1 (+311079); chr2:1608023
5-16080304/MYCN (−413); chr2:17721679-17721750/VSNL1 (−92); chr2:18060141-18060198/KCNS3 (+225); c
hr2:19550331-19550407/NT5C1B-RDH14 (−779523), OSR1 (+8003); chr2:19550546-19550611/NT5C1B-RDH1
4 (−779733), OSR1 (+7793); chr2:19551084-19551162/NT5C1B-RDH14 (−780277), OSR1 (+7249); chr2:195514
49-19551499/NT5C1B-RDH14 (−780628), OSR1 (+6898); chr2:19553219-19553428/NT5C1B-RDH14 (−782478),
OSR1 (+5048); chr2:19556065-19556244/NT5C1B-RDH14 (−785309), OSR1 (+2217); chr2:19556883-195569
80/NT5C1B-RDH14 (−786086), OSR1 (+1440); chr2:19560967-19561042/OSR1 (−2633); chr2:19561473-19561
523/OSR1 (−3126); chr2:20250980-20251147/LAPTM4A (+725); chr2:20251380-20251427/LAPTM4A (+385); c
hr2:20646774-20646843/RHOB (−26); chr2:20851054-20851110/HS1BP3 (−218); chr2:20865159-20865289/GD
F7 (−1200); chr2:20869834-20869861/GDF7 (+3424), APOB (+397097); chr2:24149879-24149938/ATAD2B (+2
7); chr2:24162361-24162431/UBXN2A (−980); chr2:24345999-24346054/PFN4 (+124); chr2:25109600-2510963
6/ADCY3 (+32437), CENPO (+93287); chr2:25390591-25390658/POMC (+934); chr2:25390759-25390811/PO
MC (+774); chr2:25433421-25433512/POMC (−41908), DNMT3A (+131307); chr2:25438878-25438923/POMC
(−47342), DNMT3A (+125873); chr2:25499964-25500051/POMC (−108449), DNMT3A (+64766); chr2:25500162-
25500197/POMC (−108621), DNMT3A (+64594); chr2:25509077-25509162/POMC (−117561), DNMT3A (+5565
4); chr2:25518105-25518160/POMC (−126574), DNMT3A (+46641); chr2:26045137-26045159/DTNB (−148645),
ASXL2 (+56164); chr2:26205465-26205497/KIF3C (−38); chr2:26396963-26397014/HADHA (+70605), RAB10
(+140260); chr2:26407339-26407688/HADHA (+60080), RAB10 (+150785); chr2:26407922-26408092/HADHA
(+59587), RAB10 (+151278); chr2:26521450-26521525/GPR113 (+20429), HADHB (+53872); chr2:26568463-2
6568495/EPT1 (−475); chr2:26568652-26568694/EPT1 (−281); chr2:26624759-26624992/EPT1 (+55922), OTO
F (+156690); chr2:26909150-26909203/CIB4 (−44966), KCNK3 (−6404); chr2:26916067-26916088/KCNK3 (+49
7); chr2:27165501-27165512/MAPRE3 (−28018), DPYSL5 (+94538); chr2:27295119-27295159/OST4 (−572); ch
r2:27309143-27309206/KHK (−436); chr2:27341401-27341457/CGREF1 (+344); chr2:27465187-27465333/SLC
30A3 (+20700), CAD (+25002); chr2:27529745-27529838/UCN (+1338), TRIM54 (+24495); chr2:27530381-275
30429/UCN (+725); chr2:27631952-27631976/PPM1G (+532); chr2:27632625-27632686/PPM1G (−160); chr2:2
7651911-27651930/NRBP1 (+736); chr2:27717708-27717747/GCKR (−1978), FNDC4 (+398); chr2:27718138-2
7718172/GCKR (−1551), FNDC4 (−29); chr2:29033893-29034051/SPDYA (+272); chr2:29338052-29338158/C2
orf71 (−40978), ALK (+806372); chr2:29338748-29338809/C2orf71 (−41652), ALK (+805698); chr2:30143059-30
143137/C2orf71 (−845971), ALK (+1379); chr2:30143323-30143382/C2orf71 (−846226), ALK (+1124); chr2:3014
4151-30144174/ALK (+314); chr2:30370238-30370350/YPEL5 (+544); chr2:30453654-30453784/LBH (−678); c
hr2:30670021-30670049/LCLAT1 (−102); chr2:30998709-30998790/CAPN13 (+31561), LCLAT1 (+328613); chr
2:31360590-31360630/GALNT14 (+982); chr2:31360756-31360803/GALNT14 (+812); chr2:31457261-3145732
5/EHD3 (+413); chr2:31805536-31805625/SRD5A2 (+459); chr2:31805765-31805974/SRD5A2 (+170); chr2:32
390662-32390708/SLC30A6 (−225); chr2:32391175-32391221/SLC30A6 (+288); chr2:32581993-32582020/BIR
C6 (−89); chr2:33172249-33172288/LTBP1 (−100); chr2:37193357-37193416/STRN (+228); chr2:38300861-383
00957/CYP1B1 (+2414), FAM82A1 (+123313); chr2:38301285-38301350/CYP1B1 (+2005), FAM82A1 (+12372
2); chr2:38301675-38301762/CYP1B1 (+1604), FAM82A1 (+124123); chr2:38302188-38302369/CYP1B1 (+104
4), FAM82A1 (+124683); chr2:38302901-38302948/CYP1B1 (+398); chr2:38830113-38830190/HNRPLL (+26);
chr2:39187044-39187092/ARHGEF33 (+40564), SOS1 (+160536); chr2:39187238-39187417/ARHGEF33 (+40
824), SOS1 (+160276); chr2:39351644-39351702/SOS1 (−4069); chr2:39892748-39892804/TMEM178 (−259); c
hr2:40679605-40679688/SLC8A1 (−22203); chr2:42325910-42326069/EML4 (−70500), PKDCC (+50829); chr2:4
2329445-42329493/EML4 (−67021), PKDCC (+54308); chr2:42587646-42587714/COX7A2L (+1308), EML4 (+1
91190); chr2:42719906-42720157/COX7A2L (−131044), KCNG3 (+1205); chr2:42720463-42720772/KCNG3
(+619); chr2:43020325-43020503/HAAO (−663); chr2:43054825-43054879/HAAO (−35101), ZFP36L2 (+398893);
chr2:43132933-43132951/HAAO (−113191), ZFP36L2 (+320803); chr2:43397997-43398117/HAAO (−378306), Z
FP36L2 (+55688); chr2:43451846-43451891/HAAO (−432118), ZFP36L2 (+1876); chr2:44001154-44001213/DY
NC2LI1 (+6); chr2:44394953-44395000/PPM1B (−1023); chr2:44588791-44588834/CAMKMT (−230), PREPL
(+188); chr2:44995688-44995716/SIX3 (−173335), CAMKMT (+406659); chr2:45028561-45028603/SIX3 (−140455),
CAMKMT (+439539); chr2:45029059-45029183/SIX3 (−139916), CAMKMT (+440078); chr2:45029779-45029
987/SIX3 (−139154), CAMKMT (+440840); chr2:45155211-45155355/SIX3 (−13754), CAMKMT (+566240); chr2:
45156812-45156832/SIX3 (−12215), CAMKMT (+567779); chr2:45161541-45161597/SIX3 (−7468), CAMKMT
(+572526); chr2:45164608-45164656/SIX3 (−4405); chr2:45168079-45168164/SIX3 (−915); chr2:45170123-45170
168/SIX3 (+1109), SIX2 (+66396); chr2:45170582-45170718/SIX3 (+1613), SIX2 (+65892); chr2:45171031-451
71096/SIX3 (+2027), SIX2 (+65478); chr2:45171564-45171836/SIX3 (+2663), SIX2 (+64842); chr2:45227553-4
5227987/SIX2 (+8772), SIX3 (+58733); chr2:45228577-45228626/SIX2 (+7940), SIX3 (+59565); chr2:45231151-
45231244/SIX2 (+5344), SIX3 (+62161); chr2:45231538-45231696/SIX2 (+4925), SIX3 (+62580); chr2:452404
27-45240490/SIX2 (−3917); chr2:45240631-45240763/SIX2 (−4155); chr2:45241169-45241232/SIX2 (−4659); chr
2:45397303-45397345/81X2 (−160782), SRBD1 (+441109); chr2:45397483-45397506/SIX2 (−160953), SRBD1
(+440938); chr2:46770252-46770288/RHOQ (+403); chr2:47206751-47206854/TTC7A (+38490), CALM2 (+196
937); chr2:47210423-47210514/TTC7A (+42156), CALM2 (+193271); chr2:47403663-47403706/CALM2 (+55);
chr2:47799033-47799123/KCNK12 (−1608); chr2:48010116-48010160/MSH6 (−83); chr2:48541436-48541485/F
OXN2 (−334); chr2:48982701-48982753/LHCGR (+153); chr2:50573377-50573484/NRXN1 (+686243); chr2:505
73639-50573691/NRXN1 (+686009); chr2:50574356-50574401/NRXN1 (+685295); chr2:50574685-50574738/N
RXN1 (+684962); chr2:51259141-51259201/NRXN1 (+503); chr2:54784837-54784958/EML6 (−167251), RPL23
AP32 (+28539); chr2:55276260-55276305/RTN4 (+1451), EML6 (+324134); chr2:55459270-55459317/C2orf63
(+155), RPS27A (+255); chr2:55921006-55921038/PNPT1 (−11); chr2:56150536-56150681/EFEMP1 (+689); ch
r2:58468230-58468297/FANCL (+251); chr2:60666900-60666928/BCL11A (+113719); chr2:60782677-6078277
7/BCL11A (−2094); chr2:60797739-60797850/PAPOLG (−185570), BCL11A (−17162); chr2:61292606-61292645/
C2orf74 (−97003), PUS10 (−47261); chr2:61404860-61404897/AHSA2 (+324); chr2:61698185-61698214/USP34
(−351); chr2:61763690-61763892/USP34 (−65942), XPO1 (+1627); chr2:61764558-61764569/XPO1 (+854); chr
2:62422471-62422497/B3GNT2 (−778); chr2:62684078-62684131/TMEM17 (+49499), B3GNT2 (+260843); chr2:
62798447-62798511/EHBP1 (−134522), TMEM17 (−64875); chr2:63274711-63275010/OTX1 (−3076); chr2:632
75192-63275326/OTX1 (−2678); chr2:63279022-63279141/OTX1 (+1145), WDPCP (+536785); chr2:63279949-
63280154/OTX1 (+2115), WDPCP (+535815); chr2:63282867-63282955/OTX1 (+4974), WDPCP (+532956); ch
r2:63286048-63286354/OTX1 (+8264), WDPCP (+529666); chr2:63286585-63286693/OTX1 (+8702), WDPCP
(+529228); chr2:63286817-63286885/OTX1 (+8914), WDPCP (+529016); chr2:64682204-64682235/LGALSL
(+893); chr2:64836433-64836470/SERTAD2 (+44594), AFTPH (+85013); chr2:64836633-64836746/SERTAD2
(+44356), AFTPH (+85251); chr2:64863609-64863650/SERTAD2 (+17416), AFTPH (+112191); chr2:65092901-6
5092951/SERTAD2 (−211880), SLC1A4 (−123569); chr2:65216166-65216199/SLC1A4 (−312); chr2:65216578-6
5216614/SLC1A4 (+101); chr2:65356961-65357006/RAB1A (+451); chr2:65357249-65357304/RAB1A (+158); c
hr2:65660591-65660676/SPRED2 (−978); chr2:66653934-66654136/SPRED2 (−994379), MEIS1 (−8497); chr2:6
6659462-66659535/MEIS1 (−3033); chr2:66666408-66666698/ETAA1 (−957889), MEIS1 (+4021); chr2:6667165
8-66671794/ETAA1 (−952716), MEIS1 (+9194); chr2:66672431-66672462/ETAA1 (−951995), MEIS1 (+9915); ch
r2:66673076-66673138/ETAA1 (−951335), MEIS1 (+10575); chr2:66673272-66673321/ETAA1 (−951145), MEIS
1 (+10765); chr2:66673591-66673634/ETAA1 (−950829), MEIS1 (+11081); chr2:66808634-66808726/ETAA1
(−815762), MEIS1 (+146148); chr2:68479126-68479170/PPP3R1 (+503); chr2:68546231-68546249/PPP3R1 (−66
589), PLEK (−46082); chr2:68546517-68546552/PPP3R1 (−66884), PLEK (−45787); chr2:68589404-68589508/P
LEK (−2866); chr2:68694060-68694138/APLF (−592); chr2:69001773-69001853/ARHGAP25 (+39900), BMP10
(+96836); chr2:69064445-69064579/BMP10 (+34137), ARHGAP25 (+102599); chr2:69534748-69534773/GFPT
1 (+79625), ANTXR1 (+294485); chr2:70370085-70370094/PCBP1 (+55505), TIA1 (+105689); chr2:71017540-
71017738/FIGLA (+136); chr2:71017851-71017960/FIGLA (−131); chr2:71113829-71113880/CD207 (−50902), V
AX2 (−13865); chr2:71114917-71114939/CD207 (−51975), VAX2 (−12792); chr2:71115097-71115165/CD207 (−5
2178), VAX2 (−12589); chr2:71115961-71116118/CD207 (−53087), VAX2 (−11680); chr2:71116232-71116335/C
D207 (−53331), VAX2 (−11436); chr2:71123991-71124072/VAX2 (−3688); chr2:71124469-71124530/VAX2 (−322
0); chr2:71127994-71128059/VAX2 (+307); chr2:71128670-71128707/VAX2 (+969); chr2:71128935-71128996/
ATP6V1B1 (−34032), VAX2 (+1246); chr2:71131848-71132150/ATP6V1B1 (−30999), VAX2 (+4279); chr2:71134
585-71134745/ATP6V1B1 (−28333), VAX2 (+6945); chr2:72371254-72371406/CYP26B1 (+3633), DYSF (+6774
98); chr2:72374526-72374563/CYP26B1 (+418); chr2:72374690-72374713/CYP26B1 (+261); chr2:72376723-7
2376765/CYP26B1 (−1781); chr2:73147528-73147966/EMX1 (+3143), SFXN5 (+151218); chr2:73148067-7314
8174/EMX1 (+3517), SFXN5 (+150844); chr2:73151884-73151946/EMX1 (+7311), SFXN5 (+147050); chr2:731
52231-73152264/EMX1 (+7644), SFXN5 (+146717); chr2:73152679-731 52739/EMX1 (+8105), SFXN5 (+14625
6); chr2:73297630-73297735/SFXN5 (+1282), EMX1 (+153079); chr2:73340372-73340425/RAB11FIP5 (−253);
chr2:73429923-73429976/NOTO (+564); chr2:73430191-73430201/NOTO (+810); chr2:73430373-73430442/S
MYD5 (−10958), NOTO (+1022); chr2:74374694-74374740/BOLA3 (+322); chr2:74405682-74405752/MOB1A
(+278); chr2:74642053-74642227/DCTN1 (−34658), C2orf81 (+2704); chr2:74642637-74642695/DCTN1 (−35184),
C2orf81 (+2178); chr2:74642811-74642871/DCTN1 (−35359), C2orf81 (+2003); chr2:74684399-74684460/WB
P1 (−1097); chr2:74710039-74710101/TTC31 (−130), CCDC142 (+287); chr2:74724941-74725058/CCDC142 (−1
4643), LBX2 (+5443); chr2:74725173-74725425/CCDC142 (−14942), LBX2 (+5144); chr2:74726448-74726598/
CCDC142 (−16166), LBX2 (+3920); chr2:74726860-74727020/CCDC142 (−16583), LBX2 (+3503); chr2:7472939
9-74729456/CCDC142 (−19071), LBX2 (+1015); chr2:74734830-74734879/LBX2 (−4412), PCGF1 (−34); chr2:74
741845-74741872/TLX2 (+263); chr2:74742166-74742256/TLX2 (+615); chr2:74742648-74742693/TLX2 (+107
5), DQX1 (+10737); chr2:74743206-74743766/TLX2 (+1890), DQX1 (+9922); chr2:74782088-74782218/LOXL3
(−1091), DOK1 (+641); chr2:74782471-74782565/LOXL3 (−1456), DOK1 (+1006); chr2:74875214-74875376/C2
orf65 (−131); chr2:75062529-75062575/POLE4 (−123223), HK2 (+2770); chr2:75788286-75788330/FAM176A
(−216); chr2:79220337-79220374/REG3G (−32470); chr2:79740016-79740063/CTNNA2 (−20); chr2:80529909-80
529985/LRRTM1 (+1540), CTNNA2 (+789887); chr2:80530328-80530408/LRRTM1 (+1119), CTNNA2 (+79030
8); chr2:80530587-80530622/LRRTM1 (+882); chr2:85361310-85361466/TCF7L1 (+805); chr2:85361841-8536
1889/TCF7L1 (+1282), TGOLN2 (+193554); chr2:85554338-85554392/TGOLN2 (+1054), TCF7L1 (+193782); c
hr2:85633145-85633252/CAPG (+4477), ELMOD3 (+51356); chr2:85645628-85645835/MAT2A (−120369), CAP
G (−8056); chr2:85811412-85811518/VAMP5 (−66); chr2:85822758-85822811/RNF181 (−52); chr2:85839139-85
839181/USP39 (−4123); chr2:85999360-85999497/ATOH8 (+18520), ST3GAL5 (+116728); chr2:86332847-863
32909/PTCD3 (−427), POLR1A (+400); chr2:86333255-86333309/PTCD3 (−23), POLR1A (−4); chr2:86422068-8
6422111/MRPL35 (−4466), IMMT (+803); chr2:87016050-87016119/RNF103 (−165085), CD8A (+2752); chr2:87
016509-87016575/RNF103 (−165542), CD8A (+2295); chr2:87017018-87017192/RNF103 (−166105), CD8A (+1
732); chr2:87036648-87036815/CD8A (−17895), CD8B (+52315); chr2:87089567-87089633/CD8B (−553); chr2:
87175577-87175643/PLGLB1 (−871996), RGPD1 (+30872); chr2:87883811-87883908/PLGLB1 (−163746), RGP
D1 (+739122); chr2:88302765-88302792/RGPD2 (−177493), SMYD1 (−64520); chr2:88470012-88470079/THNS
L2 (−2612); chr2:88751420-88751460/FOXI3 (+613); chr2:88849493-88849562/C2orf51 (+25359), EIF2AK3 (+7
7566); chr2:88926445-88926490/EIF2AK3 (+626); chr2:88926986-88927015/EIF2AK3 (+93); chr2:89064514-89
064805/RPIA (+73484); chr2:89064976-89065128/RPIA (+73876); chr2:90448043-90448081/NONE; chr2:9163
4762-91634784/ACTR3BP2 (−494386); chr2:91635003-91635152/ACTR3BP2 (−494081); chr2:91777609-91777
635/ACTR3BP2 (−351537); chr2:91873960-91874034/ACTR3BP2 (−255162); chr2:91874200-91874586/ACTR3
BP2 (−254766); chr2:91936943-91936958/ACTR3BP2 (−192208); chr2:91997573-91997593/ACTR3BP2 (−1315
76); chr2:92028067-92028226/ACTR3BP2 (−101012); chr2:92028348-92028366/ACTR3BP2 (−100802); chr2:92
230369-92230377/ACTR3BP2 (+101214); chr2:92230556-92230579/ACTR3BP2 (+101409); chr2:92231101-92
231145/ACTR3BP2 (+101964); chr2:92231334-92231384/ACTR3BP2 (+102200); chr2:95401392-95401473/TE
KT4 (−135799); chr2:95523003-95523095/TEKT4 (−14183); chr2:95523913-95524005/TEKT4 (−13273); chr2:96
011525-96011564/FAHD2A (−56903), KCNIP3 (+48473); chr2:96781984-96782022/ADRA2B (−115); chr2:96971
833-96971887/SNRNP200 (−553); chr2:97193064-97193251/NEURL3 (−22064), ARID5A (−9306); chr2:9753520
7-97535225/SEMA4C (+519); chr2:98280400-98280436/ACTR1B (+143); chr2:98698234-98698353/CNGA3 (−2
64324), TMEM131 (−85940); chr2:98962901-98962973/CNGA3 (+319); chr2:98963826-98963868/INPP4A (−97
474), CNGA3 (+1229); chr2:99346532-99346574/MGAT4A (+1036), UNC50 (+121511); chr2:99438934-994390
78/MGAT4A (−91417), TSGA10 (+332181); chr2:99439259-99439364/MGAT4A (−91723), TSGA10 (+331875); c
hr2:99553668-99553798/MGAT4A (−206144), TSGA10 (+217454); chr2:99771109-99771144/LIPT1 (−291), TS
GA10 (+60); chr2:100106642-100106710/REV1 (−196); chr2:100758956-100759013/AFF3 (−36940), LONRF2
(+180210); chr2:100938210-100938329/LONRF2 (+925); chr2:100938545-100938574/LONRF2 (+635); chr2:10
0938810-100938984/LONRF2 (+298); chr2:101033633-101033731/CHST10 (+448); chr2:101435552-10143559
0/NPAS2 (−1042); chr2:101869802-101869860/C2orf29 (+486); chr2:101958964-101958992/RNF149 (−33800),
CREG2 (+44987); chr2:102314082-102314091/MAP4K4 (−78); chr2:102804347-102804404/IL1RL2 (+943); chr
2:105275173-105275229/POU3F3 (−196768); chr2:105459152-105459902/POU3F3 (−12442); chr2:105461065-
105461163/POU3F3 (−10855); chr2:105461414-105461555/POU3F3 (−10484); chr2:105461668-105461699/PO
U3F3 (−10285); chr2:105469857-105469880/POU3F3 (−2100); chr2:105472426-105472617/POU3F3 (+553); ch
r2:105474370-105474435/MRPS9 (−180080), POU3F3 (+2434); chr2:105480025-105480109/MRPS9 (−174416),
POU3F3 (+8098); chr2:105480389-105480629/MRPS9 (−173974), POU3F3 (+8540); chr2:105484000-105484
038/MRPS9 (−170464), POU3F3 (+12050); chr2:105489981-105490035/MRPS9 (−164475), POU3F3 (+18039);
chr2:105954159-105954225/C2orf49 (+179); chr2:105979729-105979833/C2orf49 (+25768), FHL2 (+35900); c
hr2:106361770-106361856/FHL2 (−346132), NCK2 (−106391); chr2:106403029-106403039/FHL2 (−387353), NC
K2 (−65170); chr2:106810850-106810860/UXS1 (−60); chr2:107103196-107103301/RGPD3 (−18448), ST6GAL2
(+400314); chr2:107104272-107104337/RGPD3 (−19504), ST6GAL2 (+399258); chr2:107502035-107502087/R
GPD3 (−417260), ST6GAL2 (+1502); chr2:107502486-107502518/RGPD3 (−417701), ST6GAL2 (+1061); chr2:1
07502730-107502865/ST6GAL2 (+765); chr2:107503264-107503346/ST6GAL2 (+258); chr2:108602905-10860
3014/SLC5A7 (−35); chr2:109065020-109065087/GCC2 (−523); chr2:109065493-109065524/GCC2 (−68); chr2:1
09196275-109196406/LIMS1 (−74927), GCC2 (+130764); chr2:109336324-109336344/RANBP2 (+397); chr2:10
9746388-109746462/SH3RF3 (+428); chr2:110552306-110552341/LIMS3 (−103685), RGPD6 (+1989); chr2:110
969955-110970010/NPHP1 (−7344), LIMS3 (+260669); chr2:111141070-11114112/NPHP1 (−178452), LIMS3
(+89561); chr2:111141818-111141879/NPHP1 (−179210), LIMS3 (+88803); chr2:111180813-111180979/NPHP1
(−218257), LIMS3 (+49756); chr2:111181401-111181474/NPHP1 (−218799), LIMS3 (+49214); chr2:111490007-
111490023/ACOXL (−135); chr2:111563719-111563767/BCL2L11 (−314748), ACOXL (+73593); chr2:11187642
6-111876774/BCL2L11 (−1891); chr2:111876879-111877075/BCL2L11 (−1514); chr2:112124030-112124077/BC
L2L11 (+245563), ANAPC1 (+517687); chr2:112238443-112238457/BCL2L11 (+359959), ANAPC1 (+403291);
chr2:112641350-112641379/ANAPC1 (+376); chr2:112657313-112657383/TMEM87B (−155452), MERTK (+115
7); chr2:112939287-112939458/FBLN7 (+43411), ZC3H8 (+73291); chr2:113012376-113012388/ZC3H8 (+282);
chr2:113033597-113033659/ZC3H6 (+450); chr2:114033552-114033604/PAX8 (+2920), PSD4 (+102018); chr
2:114034477-114034670/PAX8 (+1924), PSD4 (+103014); chr2:114034907-114035176/PAX8 (+1456), PSD4
(+103482); chr2:114035476-114035704/PAX8 (+908); chr2:114256780-114256870/FOXD4L1 (+164); chr2:1143
61333-114361346/RABL2A (−23477), WASH2P (+7695); chr2:114513832-114513896/SLC35F5 (+536); chr2:11
5918893-115918947/DPP10 (−593); chr2:115919151-115919337/DPP10 (−269); chr2:115919594-115919784/D
PP10 (+176); chr2:118981857-118981945/INSIG2 (+135851), EN1 (+623858); chr2:118982148-118982253/INS
IG2 (+136151), EN1 (+623558); chr2:119067515-119067551/INSIG2 (+221483), EN1 (+538226); chr2:1195915
68-119591658/EN1 (+14146), INSIG2 (+745563); chr2:119592140-119592240/EN1 (+13569), INSIG2 (+746140);
chr2:119599458-119599829/EN1 (+6115), INSIG2 (+753594); chr2:119600953-119600995/EN1 (+4785), INS
IG2 (+754924); chr2:119602630-119602828/EN1 (+3030), INSIG2 (+756679); chr2:119604680-119604689/EN1
(+1074), INSIG2 (+758635); chr2:119605470-119605501/EN1 (+273); chr2:119606500-119606630/EN1 (−806);
chr2:119607624-119607847/EN1 (−1977); chr2:119610375-119610540/EN1 (−4699); chr2:119610940-1196109
98/MARCO (−88776), EN1 (−5210); chr2:119611253-119611311/MARCO (−88463), EN1 (−5523); chr2:11961448
1-119614602/MARCO (−85203), EN1 (−8783); chr2:119616552-119616781/MARCO (−83078), EN1 (−10908); chr
2:119913245-119913310/C1QL2 (+3193), MARCO (+213533); chr2:119914200-119914247/C1QL2 (+2247), M
ARCO (+214479); chr2:119914469-119914529/C1QL2 (+1972), MARCO (+214754); chr2:119915607-1199165
24/C1QL2 (+405); chr2:120281901-120281938/SCTR (+108); chr2:120770938-120770966/EPB41L5 (+348); ch
r2:121104077-121104109/INHBB (+374); chr2:121105371-121105515/GLI2 (−449424), INHBB (+1724); chr2:12
1106691-121106786/GLI2 (−448128), INHBB (+3020); chr2:121269284-121269312/GLI2 (−285569), INHBB (+1
65579); chr2:121499455-121499643/GLI2 (−55318), INHBB (+395830); chr2:121624850-121624990/GLI2 (+700
53), TFCP2L1 (+417858); chr2:121625126-121625154/GLI2 (+70273), TFCP2L1 (+417638); chr2:121625686-1
21625734/GLI2 (+70843), TFCP2L1 (+417068); chr2:122407072-122407098/CLASP1 (−33); chr2:124782193-1
24782288/CNTNAP5 (−623); chr2:127413728-127413969/GYPC (+165); chr2:127977384-127977421/CYP27C1
(−14060), ERCC3 (+74349); chr2:128180415-128180471/PROC (+4447), IWS1 (+103644); chr2:128410712-12
8410746/GPR17 (+7290), LIMS2 (+11438); chr2:128421850-128421890/LIMS2 (+297); chr2:128432694-12843
2741/SFT2D3 (−25879), LIMS2 (−10551); chr2:129076115-129076132/HS6ST1 (+47); chr2:129492224-1294923
59/HS6ST1 (−416121); chr2:129492784-129492908/HS6ST1 (−416675); chr2:129494765-129494787/HS6ST1
(−418605); chr2:130737205-130737256/RAB6C (−4); chr2:130750152-130750422/RAB6C (+13052), POTEF (+13
6508); chr2:130750760-130750810/RAB6C (+13550), POTEF (+136010); chr2:130763677-130763729/RAB6C
(+26468), POTEF (+123092); chr2:130971042-130971062/TUBA3E (−15018), CCDC115 (+128870); chr2:13098
6930-130986966/TUBA3E (−30914), CCDC115 (+112974); chr2:131113875-131113904/PTPN18 (+310); chr2:1
31280357-131280423/CFC1 (+1723), TISP43 (+23650); chr2:131280557-131280589/CFC1 (+1906), TISP43
(+23467); chr2:131285128-131285194/CFC1 (+6494), TISP43 (+18879); chr2:131285393-131285429/CFC1 (+67
44), TISP43 (+18629); chr2:131350478-131350596/CFC1B (+6714), TISP43 (+18687); chr2:131355316-13135
5558/CFC1B (+1814), TISP43 (+23587); chr2:131450454-131450514/CFC1B (−93233), GPR148 (−36159); chr2:
131451107-131451139/CFC1B (−93872), GPR148 (−35520); chr2:131594922-131594947/ARHGEF4 (−77851),
GPR148 (+108292); chr2:131595283-131595300/ARHGEF4 (−77494), GPR148 (+108649); chr2:131673982-13
1674100/PLEKHB2 (−188379), ARHGEF4 (+1255); chr2:131720732-131720786/PLEKHB2 (−141661), ARHGEF
4 (+47973); chr2:131721189-131721437/PLEKHB2 (−141107), ARHGEF4 (+48527); chr2:131721585-13172167
0/PLEKHB2 (−140792), ARHGEF4 (+48842); chr2:131792520-131792531/PLEKHB2 (−69894), ARHGEF4 (+119
740); chr2:131792747-131792920/PLEKHB2 (−69586), ARHGEF4 (+120048); chr2:131793154-131793187/PLE
KHB2 (−69249), ARHGEF4 (+120385); chr2:131797328-131797382/PLEKHB2 (−65065), ARHGEF4 (+124569);
chr2:131797568-131797750/PLEKHB2 (−64761), ARHGEF4 (+124873); chr2:131862958-131863088/PLEKHB2
(+603); chr2:132088639-132088785/WTH3DI (+33019), POTEE (+112788); chr2:132110965-132111113/WTH3
DI (+10692), POTEE (+135115); chr2:132121223-132121284/WTH3DI (+477); chr2:132121618-132121651/WT
H3DI (+96); chr2:132182470-132182562/WTH3DI (−60785), TUBA3D (−51064); chr2:132430625-132430690/GP
R39 (−743489), LOC150776 (+180272); chr2:132559349-132559390/GPR39 (−614777), LOC150776 (+308984);
chr2:132559508-132559531/GPR39 (−614627), LOC150776 (+309134); chr2:132587093-132587168/GPR39
(−587016), LOC150776 (+336745); chr2:132795223-132795260/GPR39 (−378905), LOC150776 (+544856); chr2:
132795404-132795446/GPR39 (−378722), LOC150776 (+545039); chr2:133011389-133011423/GPR39 (−1627
41), LOC150776 (+761020); chr2:133014662-133014723/GPR39 (−159454), LOC150776 (+764307); chr2:1330
33368-133033442/GPR39 (−140742), LOC150776 (+783019); chr2:133062299-133062361/GPR39 (−111817), L
OC150776 (+811944); chr2:133426014-133426128/LYPD1 (+2410), GPR39 (+251924); chr2:133428774-13342
8819/LYPD1 (−316); chr2:136499679-136499730/UBXN4 (+516); chr2:136743162-136743181/DARS (+50); chr
2:137522750-137522848/CXCR4 (−647074), THSD7B (−225663); chr2:137523393-137523516/CXCR4 (−647730),
THSD7B (−225007); chr2:139537823-139537850/NXPH2 (−26); chr2:144694515-144694553/GTDC1 (+39554
9), ARHGAP15 (+807635); chr2:145090318-145090363/GTDC1 (−258); chr2:145274511-145274576/GTDC1 (−1
84461), ZEB2 (+3414); chr2:149632858-149632888/KIF5C (+81); chr2:149684868-149684973/LYPD6B (−2100
60), KIF5C (+52129); chr2:153573899-153573933/ARL6IP6 (−491), PRPF40A (+59); chr2:153574916-15357499
6/PRPF40A (−981), ARL6IP6 (+549); chr2:154334805-154335018/RPRM (+410); chr2:154727992-154728244/G
ALNT13 (−308); chr2:154728441-154728503/GALNT13 (+46); chr2:154728905-154729030/GALNT13 (+542); c
hr2:154729316-154729379/GALNT13 (+922); chr2:155554016-155554076/KCNJ3 (−1047); chr2:155555754-15
5555817/KCNJ3 (+693); chr2:155555926-155556041/KCNJ3 (+891); chr2:157184831-157184894/NR4A2 (+44
24); chr2:157185657-157185700/NR4A2 (+3608); chr2:157185999-157186024/NR4A2 (+3275); chr2:15718614
0-157186194/NR4A2 (+3120); chr2:158484919-158484949/ACVR1C (+465); chr2:160472404-160472575/BAZ2
B (+622); chr2:160760899-160760959/LY75-CD302 (+338); chr2:160778391-160778468/LY75-CD302 (−17163),
PLA2R1 (+140696); chr2:161263830-161263896/ITGB6 (−207274), RBMS1 (+86455); chr2:161264355-16126
4419/ITGB6 (−207798), RBMS1 (+85931); chr2:162273315-162273382/TBR1 (+729); chr2:162275227-1622753
10/SLC4A10 (−205576), TBR1 (+2649); chr2:162275438-162275472/SLC4A10 (−205390), TBR1 (+2835); chr2:1
62280482-162280740/SLC4A10 (−200234), TBR1 (+7991); chr2:162283704-1 62283765/SLC4A10 (−197110), T
BR1 (+11115); chr2:162284508-162284643/SLC4A10 (−196269), TBR1 (+11956); chr2:162930285-162930338/
DPP4 (+740); chr2:163200619-163200670/GCA (+62); chr2:163200775-163200820/GCA (+215); chr2:1636951
89-163695246/KCNH7 (+39); chr2:163695653-163695679/KCNH7 (−409); chr2:164591792-164591863/FIGN
(+685); chr2:166650410-166650454/GALNT3 (+371); chr2:167232988-167233063/SCN9A (−529); chr2:16746715
3-167467174/XIRP2 (−277833), SCN7A (−123683); chr2:169103673-169103693/STK39 (+422); chr2:169104282-
169104341/STK39 (−207); chr2:170625210-170625345/SSB (−30044), PHOSPHO2 (+74314); chr2:171564690-
171564756/SP5 (−7134), MYO3B (+530068); chr2:171570062-171570181/SP5 (−1735); chr2:171572949-17157
3231/GAD1 (−100110), SP5 (+1233); chr2:171627313-171627346/GAD1 (−45870), SP5 (+55473); chr2:1716704
47-171670471/GAD1 (−2741); chr2:171671790-171671799/GAD1 (−1405); chr2:171675383-171675522/GORAS
P2 (−109495), GAD1 (+2253); chr2:171676416-171676521/GORASP2 (−108479), GAD1 (+3269); chr2:1716767
87-171676857/GORASP2 (−108126), GAD1 (+3622); chr2:171678223-171678293/GORASP2 (−106690), GAD1
(+5058); chr2:171678684-171678980/GORASP2 (−106116), GAD1 (+5632); chr2:171679674-171679779/GORA
SP2 (−105221), GAD1 (+6527); chr2:171680032-171680071/GORASP2 (−104896), GAD1 (+6852); chr2:172865
042-172865172/METAP1D (+303); chr2:172947974-172948171/DLX1 (−2135); chr2:172949283-172949348/DL
X1 (−892); chr2:172951543-172951779/DLX1 (+1453), DLX2 (+15817); chr2:172952882-172952992/DLX1 (+27
29), DLX2 (+14541); chr2:172953096-172953124/DLX1 (+2902), DLX2 (+14368); chr2:172954917-172954947/
DLX1 (+4724), DLX2 (+12546); chr2:172955403-172955471/DLX1 (+5229), DLX2 (+12041); chr2:172961834-1
72961876/DLX2 (+5623), DLX1 (+11647); chr2:172964997-172965018/DLX2 (+2470), DLX1 (+14800); chr2:17
2965299-172965581/DLX2 (+2038), DLX1 (+15232); chr2:172965763-172965958/DLX2 (+1617), DLX1 (+1565
3); chr2:172972891-172972930/ITGA6 (−319403), DLX2 (−5433); chr2:173941263-173941327/ZAK (+730); chr2:
174219266-174219326/CDCA7 (−265); chr2:174877712-174877854/SP3 (−47353), OLA1 (+235582); chr2:1751
91973-175192084/OLA1 (−78664), SP9 (−7792); chr2:175193645-175193808/OLA1 (−80362), SP9 (−6094); chr2:
175195859-175195948/SP9 (−3917); chr2:175198592-175198658/SP9 (−1196); chr2:175198767-175198845/SP
9 (−1015); chr2:175199600-175199658/SP9 (−192); chr2:175200441-175200495/SP9 (+647); chr2:175200853-1
75200916/SP9 (+1064), CIR1 (+59558); chr2:175201183-175201309/SP9 (+1425), CIR1 (+59197); chr2:17520
1829-175202126/SP9 (+2157), CIR1 (+58465); chr2:175202276-175202351/SP9 (+2493), CIR1 (+58129); chr2:
175202601-175202633/SP9 (+2796), CIR1 (+57826); chr2:175205048-175205111/SP9 (+5259), CIR1 (+55363);
chr2:175205498-175205587/SP9 (+5722), CIR1 (+54900); chr2:175206906-175206960/SP9 (+7112), CIR1
(+53510); chr2:175208869-175208996/SP9 (+9112), CIR1 (+51510); chr2:175595007-175595072/WIPF1 (−95733),
CHRNA1 (+34160); chr2:176032904-176032922/ATF2 (−16); chr2:176931460-176931580/KIAA1715 (−64502),
HOXD10 (−49972), HOXD11 (−40564), HOXD12 (−33010), HOXD13 (−26012), EVX2 (+17170); chr2:176931688-
176932059/KIAA1715 (−64856), HOXD10 (−49618), HOXD11 (−40210), HOXD12 (−32656), HOXD13 (−25658),
EVX2 (+16816); chr2:176932437-176932483/KIAA1715 (−65442), HOXD10 (−49032), HOXD11 (−39624), HOXD
12 (−32070), HOXD13 (−25072), EVX2 (+16230); chr2:176936300-176936435/KIAA1715 (−69350), HOXD10 (−4
5124), HOXD11 (−35716), HOXD12 (−28162), HOXD13 (−21164), EVX2 (+12322); chr2:176944405-176944456/
KIAA1715 (−77413), HOXD10 (−37061), HOXD11 (−27653), HOXD12 (−20099), HOXD13 (−13101), EVX2 (+4259);
chr2:176944870-176945013/KIAA1715 (−77924), HOXD10 (−36550), HOXD11 (−27142), HOXD12 (−19588), H
OXD13 (−12590), EVX2 (+3748); chr2:176946868-176947189/KIAA1715 (−80011), HOXD10 (−34463), HOXD11
(−25055), HOXD12 (−17501), HOXD13 (−10503), EVX2 (+1661); chr2:176947654-176947763/HOXD10 (−33783),
HOXD11 (−24375), HOXD12 (−16821), HOXD13 (−9823), EVX2 (+981); chr2:176948015-176948351/HOXD10
(−33309), HOXD11 (−23901), HOXD12 (−16347), HOXD13 (−9349), EVX2 (+507); chr2:176950002-176950074/HO
XD10 (−31454), HOXD11 (−22046), HOXD12 (−14492), HOXD13 (−7494), EVX2 (−1348); chr2:176950761-17695
0824/HOXD10 (−30699), HOXD11 (−21291), HOXD12 (−13737), HOXD13 (−6739), EVX2 (−2103); chr2:17695219
1-176952318/HOXD10 (−29237), HOXD11 (−19829), HOXD12 (−12275), HOXD13 (−5277), EVX2 (−3565); chr2:1
76956600-176956644/HOXD10 (−24870), HOXD11 (−15462), HOXD12 (−7908), HOXD13 (−910); chr2:17695741
5-176957576/HOXD10 (−23996), HOXD11 (−14588), HOXD12 (−7034), HOXD13 (−36); chr2:176957685-176957
820/HOXD10 (−23739), HOXD11 (−14331), HOXD12 (−6777), HOXD13 (+221); chr2:176963254-176963321/HO
XD10 (−18204), HOXD11 (−8796), HOXD12 (−1242); chr2:176964150-176964179/HOXD10 (−17327), HOXD11
(−7919), HOXD12 (−365); chr2:176964334-176964389/HOXD10 (−17130), HOXD11 (−7722), HOXD12 (−168); chr2:
176964768-176965264/HOXD10 (−16476), HOXD11 (−7068), HOXD12 (+486); chr2:176969614-176969676/HO
XD10 (−11847), HOXD11 (−2439); chr2:176971652-176972103/HOXD10 (−9614), HOXD11 (−206); chr2:1769726
72-176972684/HOXD10 (−8814), HOXD11 (+594); chr2:176973262-176973276/HOXD10 (−8223), HOXD11 (+1
185); chr2:176973565-176973598/HOXD10 (−7910), HOXD11 (+1498); chr2:176976816-176976942/HOXD10
(−4613); chr2:176979706-176979772/HOXD10 (−1753); chr2:176980938-176981355/HOXD10 (−345); chr2:17698
2420-176982517/HOXD9 (−4944), HOXD10 (+977); chr2:176986498-176986711/HOXD9 (−808); chr2:17698740
3-176987735/HOXD9 (+156); chr2:176988104-176988154/HOXD9 (+716); chr2:176992950-176993058/HOXD8
(−1464); chr2:176994380-176994497/HOXD8 (−29); chr2:176994687-176994763/HOXD8 (+257); chr2:1769948
89-176995061/HOXD8 (+507); chr2:177001696-177001781/HOXD4 (−14374), HOXD8 (+7271); chr2:17700358
9-177003734/HOXD4 (−12451), HOXD8 (+9194); chr2:177004498-177004555/HOXD4 (−11586), HOXD8 (+100
59); chr2:177014471-177014540/HOXD4 (−1607); chr2:177016506-177016594/HOXD4 (+437); chr2:177017192-
177017269/HOXD3 (−11574), HOXD4 (+1118); chr2:177022563-177022645/HOXD3 (−6201), HOXD4 (+6491);
chr2:177024768-177024835/HOXD3 (−4003); chr2:177025058-177025197/HOXD3 (−3677); chr2:177027439-17
7027475/HOXD3 (−1348); chr2:177027834-177027930/HOXD3 (−923); chr2:177029654-177029847/HOXD3 (+9
46); chr2:177030227-177030287/HOXD1 (−23050), HOXD3 (+1452); chr2:177036363-177036880/HOXD1 (−166
85), HOXD3 (+7817); chr2:177040152-177040225/HOXD1 (−13118), HOXD3 (+11384); chr2:177042803-17704
2913/HOXD1 (−10449), HOXD3 (+14053); chr2:177043194-177043220/HOXD1 (−10100), HOXD3 (+14402); chr
2:177053435-177053618/HOXD1 (+220); chr2177053762-177053846/HOXD1 (+497); chr2:177053954-177053
999/HOXD1 (+670); chr2:177054495-177054648/MTX2 (−79551), HOXD1 (+1265); chr2:177873450-177873566/
HNRNPA3 (−203914), MTX2 (+739385); chr2:179060032-179060095/OSBPL6 (+856); chr2:179315947-179315
976/DFNB59 (−201), PRKRA (−4); chr2:180129078-180129185/SESTD1 (+218); chr2:180871633-180871654/C
WC22 (+136); chr2:181844791-181844867/UBE2E3 (−283); chr2:181845374-181845393/UBE2E3 (+272); chr2:
181845773-181845792/UBE2E3 (+671); chr2:182321838-182322038/ITGA4 (+319); chr2:182322192-1823223
99/ITGA4 (+677); chr2:182545948-182546001/NEUROD1 (−583); chr2:182546535-182546599/NEUROD1 (−117
5); chr2:182547388-182547437/NEUROD1 (−2021); chr2:182547932-182548061/NEUROD1 (−2605); chr2:1825
48789-182548819/NEUROD1 (−3412); chr2:182549026-182549037/NEUROD1 (−3640); chr2:182550007-18255
0053/NEUROD1 (−4638); chr2:183731332-183731466/FRZB (+99); chr2:183902402-183902442/NCKAP1 (+80
7); chr2:183903010-183903037/NCKAP1 (+205); chr2:191045194-191045258/MSTN (−117771), HIBCH (+1395
45); chr2:191184536-191184570/HIBCH (+218); chr2:191745125-191745184/GLS (−392); chr2:191884945-191
884987/STAT1 (−5990), STAT4 (+131356); chr2:191885316-191885345/STAT1 (−6355), STAT4 (+130991); chr
2:192110556-192110584/MYO1B (+463); chr2:193059318-193059344/TMEFF2 (+313); chr2:193059549-19305
9661/TMEFF2 (+39); chr2:197664225-197664264/GTF3C3 (+247); chr2:198380734-198380792/HSPE1-MOB4
(+16042), RFTN2 (+159821); chr2:198569960-198569977/MARS2 (−59); chr2:198570083-198570108/MARS2
(+68); chr2:198650908-198651001/BOLL (−17); chr2:198669051-198669112/PLCL1 (−344); chr2:200327254-200
327293/SATB2 (+2557); chr2:200327452-200327575/SATB2 (+2317); chr2:200328685-200328877/SATB2 (+1
050); chr2:200329394-200329432/SATB2 (+418); chr2:200330872-200330907/SATB2 (−1059); chr2:200333759-
200333800/SATB2 (−3949); chr2:200334426-200334504/SATB2 (−4634); chr2:200335452-200335591/SATB2
(−5691), FONG (+380374); chr2:201374663-201374677/KCTD18 (+122); chr2:201450922-201451013/AOX1 (+2
37); chr2:201728922-201728962/CLK1 (+525); chr2:201981462-201981485/CFLAR (+597); chr2:202316252-20
2316318/STRADB (−107), TRAK2 (+34); chr2:202507324-202507367/TMEM237 (+878); chr2:202508108-20250
8147/TMEM237 (+96); chr2:202646120-202646140/ALS2 (−235); chr2:202900051-202900071/FZD7 (+751); chr
2:205409970-205410026/PARD3B (−518); chr2:206549845-206549882/NRP2 (+2640), INO80D (+401042); chr
2:206550818-206550889/NRP2 (+3630), INO80D (+400052); chr2:206551015-206551071/NRP2 (+3819), INO8
0D (+399863); chr2:206551363-206551450/NRP2 (+4183), INO80D (+399499); chr2:207139155-207139217/Z
DBF2 (−337); chr2:207307478-207307547/ADAM23 (−855); chr2:208395895-208395934/CREB1 (+1299), METT
L21A (+94058); chr2:208546288-208546339/METTL21A (−56341), CCNYL1 (−29950); chr2:208634663-208634
701/FZD5 (−539); chr2:208989065-208989293/CRYGD (+134); chr2:209224515-209224574/PTH2R (−47011), P
IKFYVE (+93554); chr2:209271337-209271663/PTH2R (−56); chr2:209271888-209271949/PTH2R (+363); chr2:
210636382-210636429/UNC80 (−311); chr2:210636690-210636737/UNC80 (−3); chr2:210636877-210636933/U
NC80 (+188); chr2:211089487-211089524/ACADL (+709); chr2:217236188-217236231/MARCH4 (+540); chr2:
217498269-217498283/IGFBP2 (+149); chr2:218650360-218650426/TNP1 (−925611), TNS1 (+158403); chr2:2
19232113-219232221/TMBIM1 (−74887), SLC11A1 (−14585); chr2:219232560-219232655/TMBIM1 (−75328), S
LC11A1 (−14144); chr2:219233607-219233652/TMBIM1 (−76350), SLC11A1 (−13122); chr2:219264531-219264
572/CTDSP1 (+74); chr2:219271337-219271395/VIL1 (−12472), CTDSP1 (+6888); chr2:219738412-219738596/
WNT10A (−6751), WNT6 (+13958); chr2:219825093-219825130/CDK5R2 (+714); chr2:219846871-219846894/
FEV (+3496), CDK5R2 (+22485); chr2:219848892-219848935/FEV (+1465), CDK5R2 (+24516); chr2:21985773
8-219857780/CRYBA2 (+368); chr2:219922230-219922497/CCDC108 (−16119), IHH (+2874); chr2:219924393-
219924438/IHH (+822); chr2:220025097-220025161/NHEJ1 (+458); chr2:220071383-220071429/ZFAND2B (−1
32); chr2:220083645-220083700/ABCB6 (+1501), ZFAND2B (+12135); chr2:220143946-220144004/DNAJB2
(−65); chr2:220159796-220159848/PTPRN (+14473), DNAJB2 (+15782); chr2:220173611-220173681/PTPRN
(+649); chr2:220174037-220174059/PTPRN (+247); chr2:220223700-220223794/RESP18 (−25848), DNPEP (+28
915); chr2:220253314-220253362/DNPEP (−676); chr2:220283131-220283172/DES (+53); chr2:220283290-220
283362/DES (+227); chr2:220283626-220283711/DES (+570); chr2:220299635-220299885/SPEG (+60); chr2:2
20313141-220313446/GMPPA (−50293), SPEG (+13594); chr2:220313677-220313727/GMPPA (−49885), SPE
G (+14002); chr2:220313910-220313944/GMPPA (−49660), SPEG (+14227); chr2:220348984-220349054/GMP
PA (−14568), SPEG (+49319); chr2:220361546-220361627/GMPPA (−2000); chr2:220361902-220361983/GMP
PA (−1644); chr2:220362400-220362444/GMPPA (−1165); chr2:220362756-220362787/GMPPA (−815); chr2:22
0387213-220387271/ACCN4 (+8350), CHPF (+21245); chr2:220493870-220493936/SLC4A3 (+1611); chr2:223
159806-223159868/EPHA4 (−722827), PAX3 (+3878); chr2:223160202-223160250/EPHA4 (−723216), PAX3
(+3489); chr2:223161685-223161806/EPHA4 (−724736), PAX3 (+1969); chr2:223162891-223162928/PAX3 (+805);
chr2:223163225-223163472/PAX3 (+366); chr2:223165841-223165871/PAX3 (−2141); chr2:223166226-2231
66293/PAX3 (−2545); chr2:223168832-223168916/SGPP2 (−120448), PAX3 (−5159); chr2:223175695-22317574
5/SGPP2 (−113602), PAX3 (−12005); chr2:223176512-223176719/SGPP2 (−112706), PAX3 (−12901); chr2:2231
77208-223177244/SGPP2 (−112096), PAX3 (−13511); chr2:225906779-225906790/DOCK10 (+545); chr2:22590
7362-225907439/DOCK10 (−71); chr2:228029351-228029372/COL4A4 (−87), COL4A3 (+81); chr2:228582796-2
28582819/SLC19A3 (−63); chr2:228736296-228736335/CCL20 (+57758), SPHKAP (+310045); chr2:229046745-
229046803/SPHKAP (−413); chr2:230579343-230579375/DNER (−73); chr2:230579856-230579910/DNER (−59
7); chr2:230933429-230933493/SLC16A14 (+254); chr2:231855584-231855597/GPR55 (−65650), SPATA3 (−52
48); chr2:232055534-232055613/HTR2B (−65750), ARMC9 (−7768); chr2:232236066-232236125/B3GNT7 (−242
39), ARMC9 (+172754); chr2:232253186-232253251/B3GNT7 (−7116), ARMC9 (+189877); chr2:232260942-23
2260984/B3GNT7 (+628); chr2:232395022-232395054/NMUR1 (+144); chr2:232645692-232645729/PDE6D
(+263); chr2:233216688-233216695/ALPP (−26656), DIS3L2 (+390399); chr2:233350540-233350843/ECEL1 (+18
40), ALPI (+29859); chr2:233351279-233351415/ECEL1 (+1185), ALPI (+30514); chr2:233351734-233351789/
ECEL1 (+770); chr2:233352007-233352101/ECEL1 (+478); chr2:233352452-233352506/ECEL1 (+53); chr2:23
3352763-233352775/ECEL1 (−237); chr2:233498180-233498345/EFHD1 (+56); chr2:233498527-233498571/EF
HD1 (+342); chr2:233498874-233498895/EFHD1 (+678); chr2:233499345-233499385/GIGYF2 (−62650), EFHD
1 (+1158); chr2:233756005-233756150/C2orf82 (+21084), NGEF (+121873); chr2:233793223-233793480/C2orf
82 (+58358), NGEF (+84599); chr2:233925164-233925278/INPP5D (+185); chr2:233928607-233928760/ATG1
6L1 (−231533), INPP5D (+3648); chr2:233949316-233949409/ATG16L1 (−210854), INPP5D (+24327); chr2:234
262733-234262773/DGKD (−400); chr2:234475246-234475309/USP40 (−1042); chr2:234475412-234475473/US
P40 (−1207); chr2:234674909-234674929/HEATR7B1 (−62282), UGT1A1 (+6000); chr2:234763024-234763063/
HJURP (+168); chr2:234847791-234847855/SPP2 (−111523), TRPM8 (+21780); chr2:235404533-235404550/A
RL4C (+1151), SPP2 (+445196); chr2:236163957-236164001/AGAP1 (−238754), SH3BP4 (+303351); chr2:236
401803-236401897/AGAP1 (−883); chr2:236579293-236579497/AGAP1 (+176662), GBX2 (+497257); chr2:236
579598-236579694/AGAP1 (+176913), GBX2 (+497006); chr2:236579804-236579913/AGAP1 (+177126), GBX
2 (+496793); chr2:236962012-236962058/GBX2 (+114617), AGAP1 (+559302); chr2:237067666-237067694/G
BX2 (+8972), AGAP1 (+664947); chr2:237068094-237068146/GBX2 (+8532), AGAP1 (+665387); chr2:2370685
53-237068626/GBX2 (+8062), AGAP1 (+665857); chr2:237068994-237069133/GBX2 (+7588), AGAP1 (+66633
1); chr2:237071724-237072007/GBX2 (+4786), AGAP1 (+669133); chr2:237072323-237072393/GBX2 (+4294),
AGAP1 (+669625); chr2:237072601-237072641/GBX2 (+4031), AGAP1 (+669888); chr2:237073015-23707305
6/GBX2 (+3616), AGAP1 (+670303); chr2:237073496-237073564/GBX2 (+3122), AGAP1 (+670797); chr2:2370
74563-237074633/GBX2 (+2054), AGAP1 (+671865); chr2:237076636-237076656/GBX2 (+6); chr2:237077006-
237077033/GBX2 (−368); chr2:237077240-237077284/GBX2 (−610); chr2:237078703-237078732/GBX2 (−2066);
chr2:237081554-237081748/GBX2 (−4999); chr2:237082263-237082343/GBX2 (−5651), ASB18 (+90685); chr
2:237145749-237145963/GBX2 (−69204), ASB18 (+27132); chr2:237415955-237416070/IQCA1 (+79); chr2:238
396154-238396164/MLPH (+281); chr2:238535731-238535875/LRRFIP1 (−65004), RAB17 (−36034); chr2:2385
35985-238536004/LRRFIP1 (−64812), RAB17 (−36226); chr2:238600057-238600103/LRRFIP1 (−727); chr2:238
864631-238864726/UBE2F (−10908), RAMP1 (+96492); chr2:239072391-239072429/FAM132B (−223); chr2:23
9130005-239130042/ILKAP (−17700), HES6 (+18657); chr2:239139910-239140138/ILKAP (−27700), HES6 (+86
57); chr2:239143049-239143122/ILKAP (−30762), HES6 (+5595); chr2:239755198-239755413/TWIST2 (−1367);
chr2:239755659-239755668/TWIST2 (−1009); chr2:239756066-239756230/TWIST2 (−525); chr2:239756463-23
9756487/TWIST2 (−198); chr2:239756608-239756633/TWIST2 (−52); chr2:239756765-239756815/TWIST2 (+11
7); chr2:239757731-239757898/TWIST2 (+1142), HDAC4 (+564828); chr2:239758061-239758125/TWIST2 (+1
420), HDAC4 (+564550); chr2:239836625-239836700/TWIST2 (+79990), HDAC4 (+485980); chr2:240188997-2
40189050/HDAC4 (+133619), TWIST2 (+432351); chr2:240302575-240302617/HDAC4 (+20047), TWIST2 (+54
5923); chr2:240754392-240754468/HDAC4 (−431787), NDUFA10 (+210389); chr2:241375642-241375691/GPC
1 (+552); chr2:241393167-241393198/GPC1 (+18068), ANKMY1 (+104222); chr2:241393815-241394001/GPC
1 (+18793), ANKMY1 (+103497); chr2:241395263-241395453/GPC1 (+20243), ANKMY1 (+102047); chr2:2415
00005-241500039/ANKMY1 (−2617), DUSP28 (+551); chr2:241505187-241505272/RNPEPL1 (−2774); chr2:241
612445-241612516/AQP12B (+9836), GPR35 (+47819); chr2:241759008-241759042/KIF1A (+700); chr2:24185
5419-241855455/SNED1 (−82818), AGXT (+47275); chr2:241855730-241855768/SNED1 (−82506), AGXT (+47
587); chr2:241855999-241856135/SNED1 (−82188), AGXT (+47905); chr2:241905139-241905250/SNED1 (−330
60), AGXT (+97033); chr2:242143870-242143956/ANO7 (+15989), HDLBP (+68333); chr2:242480994-2424814
37/STK25 (−33182), BOK (−16930); chr2:242640669-242640751/ING5 (−746); chr2:242674490-242674559/D2H
GDH (+495); chr2:242743101-242743395/NEU4 (−6912), GAL3ST2 (+27008); chr2:242744064-242744112/NE
U4 (−6072), GAL3ST2 (+27848); chr2:242744471-242744588/NEU4 (−5630), GAL3ST2 (+28290); chr2:2427595
31-242759692/NEU4 (+9452), PDCD1 (+41446); chr2:242784909-242785075/PDCD1 (+16066), NEU4 (+34832);
chr2:242805778-242805890/PDCD1 (−4776); chr20:291163-291220/SOX12 (−15047), ZCCHC3 (+12988); chr
20:590599-590871/TCF15 (+175); chr20:591075-591135/TCF15 (−195); chr20:592560-592730/TCF15 (−1735);
chr20:634328-634362/SRXN1 (−455); chr20:644352-644406/SRXN1 (−10489), SCRT2 (+12444); chr20:644826-
644985/SRXN1 (−11016), SCRT2 (+11917); chr20:708902-708944/SCRT2 (−52100), C20orf54 (+40305); chr20:
822197-822314/FAM110A (+7900), ANGPT4 (+74704); chr20:822900-822973/FAM110A (+8581), ANGPT4 (+7
4023); chr20:826022-826132/FAM110A (+11721), ANGPT4 (+70883); chr20:1784306-1784369/SIRPG (−14591
3), SIRPA (−91087); chr20:1851911-1852013/SIRPG (−213537), SIRPA (−23463); chr20:1874484-1874648/SIRP
A (−859); chr20:1875120-1875155/SIRPA (−287); chr20:1946353-1946430/PDYN (+28002), SIRPA (+70967); ch
r20:2539133-2539193/NOP56 (−94015), TMC2 (+21910); chr20:2633168-2633217/NOP56 (+15); chr20:267376
6-2673832/EBF4 (+275); chr20:2674457-2674614/EBF4 (+1012), CPXM1 (+106756); chr20:2730186-2730195/
CPXM1 (+51101), EBF4 (+56667); chr20:2731244-2731391/CPXM1 (+49974), EBF4 (+57794); chr20:2785867-
2785955/CPXM1 (−4619); chr20:2801733-2801857/TMEM239 (+4819), FAM113A (+19537); chr20:3052306-30
52396/OXT (+85); chr20:3063005-3063059/AVP (+2338), OXT (+10766); chr20:3063827-3063846/AVP (+1533),
OXT (+11571); chr20:3064000-3064058/AVP (+1341), OXT (+11763); chr20:3073392-3073560/AVP (−8106),
UBOX5 (+67064); chr20:3074094-3074226/AVP (−8790), UBOX5 (+66380); chr20:3204137-3204229/ITPA (+14
127), SLC4A11 (+14652); chr20:3229480-3229526/ATRN (−222162), SLC4A11 (−10668); chr20:3387181-33872
58/SLC4A11 (−168385), ATRN (−64445); chr20:3388355-3388397/SLC4A11 (−169541), ATRN (−63289); chr20:3
389372-3389571/SLC4A11 (−170637), ATRN (−62193); chr20:3641225-3641310/GFRA4 (+2778), ATRN (+1896
03); chr20:3641548-3641773/GFRA4 (+2385), ATRN (+189996); chr20:3653133-3653532/GFRA4 (−9287), ADA
M33 (+9405); chr20:3801152-3801162/C20orf29 (−14); chr20:3869459-3869510/PANK2 (−257); chr20:3869921-
3869944/PANK2 (+191); chr20:4135706-4135816/SMOX (+6311), ADRA1D (+93898); chr20:4142684-4142806/
SMOX (+13295), ADRA1D (+86914); chr20:4741367-4741409/PRND (+38832), RASSF2 (+54381); chr20:5297
048-5297225/PROKR2 (−2114); chr20:5297419-5297672/PROKR2 (−2523); chr20:6750700-6750925/BMP2 (+2
068); chr20:8000360-8000516/TMX4 (−45); chr20:9487146-9487211/LAMP5 (−7826), PLCB4 (+198732); chr20:
9487720-9487788/LAMP5 (−7251), PLCB4 (+199307); chr20:9488282-9488328/LAMP5 (−6700), PLCB4 (+1998
58); chr20:9496531-9496580/LAMP5 (+1551), PAK7 (+323131); chr20:9819324-9819466/PAK7 (+292); chr20:9
819788-9819833/PAK7 (−124); chr20:10198731-10198940/SNAP25 (−641); chr20:10200115-10200153/SNAP25
(+657); chr20:10200327-10200350/SNAP25 (+862); chr20:11667702-11667739/NONE; chr20:11667938-11667
967/NONE; chr20:13975729-13975784/SEL1L2 (−4495), MACROD2 (−389); chr20:17208657-17208739/PCSK2
(+1101), BFSP1 (+303315); chr20:17661972-17661995/RRBP1 (+944); chr20:17840265-17840349/SNX5 (+10
8847), BANF2 (+165987); chr20:18038167-18038209/OVOL2 (+333); chr20:18038338-18038356/OVOL2 (+174);
chr20:19739352-19739431/RIN2 (−127773), SLC24A3 (+546102); chr20:19739566-19739612/RIN2 (−127576),
SLC24A3 (+546299); chr20:20345631-20346136/INSM1 (−2881); chr20:20347710-20347736/INSM1 (−1042); c
hr20:20348222-20348257/INSM1 (−525); chr20:20433164-20433287/INSM1 (+84461), RALGAPA2 (+260040);
chr20:20692981-20693043/RALGAPA2 (+254); chr20:21000901-21001131/RALGAPA2 (−307750), PLK1S1 (−1
05608); chr20:21080957-21081028/RALGAPA2 (−387727), PLK1S1 (−25631); chr20:21081853-21082009/RALG
APA2 (−388665), PLK1S1 (−24693); chr20:21082124-21082215/RALGAPA2 (−388904), PLK1S1 (−24454); chr20:
21085711-21085767/RALGAPA2 (−392473), PLK1S1 (−20885); chr20:21086152-21086194/RALGAPA2 (−3929
07), PLK1S1 (−20451); chr20:21283859-21283912/XRN2 (−56); chr20:21372193-21372294/NKX2-4 (+5803), XR
N2 (+88302); chr20:21376429-21376589/NKX2-4 (+1538), XRN2 (+92567); chr20:21376734-21376876/NKX2-4
(+1242), XRN2 (+92863); chr20:21377129-21377473/NKX2-4 (+746); chr20:21377641-21377737/NKX2-4 (+35
8); chr20:21377935-21378153/NKX2-4 (+3); chr20:21486660-21486785/NKX2-4 (−108676), NKX2-2 (+7941); ch
r20:21486917-21486954/NKX2-4 (−108889), NKX2-2 (+7728); chr20:21488514-21488591/NKX2-4 (−110506), N
KX2-2 (+6111); chr20:21489159-21489239/NKX2-4 (−111152), NKX2-2 (+5465); chr20:21489447-21489621/NK
X2-4 (−111487), NKX2-2 (+5130); chr20:21490763-21490814/NKX2-4 (−112742), NKX2-2 (+3875); chr20:21491
723-21491742/NKX2-4 (−113686), NKX2-2 (+2931); chr20:21492263-21492308/NKX2-4 (−114239), NKX2-2 (+2
378); chr20:21492410-21492507/NKX2-4 (−114412), NKX2-2 (+2205); chr20:21492826-21492990/NKX2-4 (−114
861), NKX2-2 (+1756); chr20:21493994-21494360/NKX2-2 (+487); chr20:21496478-21496683/NKX2-2 (−1917);
chr20:21499106-21499132/NKX2-2 (−4455); chr20:21501360-21501444/PAX1 (−184895), NKX2-2 (−6738); chr2
0:21502145-21502443/PAX1 (−184003), NKX2-2 (−7630); chr20:21502700-21502837/PAX1 (−183528), NKX2-2
(−8105); chr20:21503208-21503230/PAX1 (−183078), NKX2-2 (−8555); chr20:21682329-21682361/PAX1 (−3952);
chr20:21683772-21683899/PAX1 (−2461); chr20:21684210-21684347/PAX1 (−2018); chr20:21685833-216858
86/PAX1 (−437); chr20:21687383-21687755/PAX1 (+1272), FOXA2 (+877532); chr20:21695274-21695305/PAX
1 (+8993), FOXA2 (+869811); chr20:22549141-22549193/FOXA2 (+15934), PAX1 (+862870); chr20:22562509-
22562568/FOXA2 (+2562), PAX1 (+876242); chr20:22562886-22563238/FOXA2 (+2039), PAX1 (+876765); chr
20:22563636-22563663/FOXA2 (+1451), PAX1 (+877353); chr20:22565602-22565634/FOXA2 (−517); chr20:22
567565-22567605/FOXA2 (−2484); chr20:22567834-22567876/FOXA2 (−2754); chr20:23015769-23015818/SST
R4 (−263); chr20:23016007-23016088/SSTR4 (−9); chr20:23028641-23028676/THBD (+1642), SSTR4 (+12602);
chr20:23029240-23029588/THBD (+887); chr20:23030086-23030291/THBD (+112); chr20:23066831-2306687
4/CD93 (+124); chr20:23067125-23067169/CD93 (−170); chr20:23338777-23338824/GZF1 (−6199), NXT1 (+74
28); chr20:23636302-23636380/CST3 (−17767), CST4 (+33321); chr20:23636687-23636782/CST3 (−18161), CS
T4 (+32927); chr20:24449703-24449759/SYNDIG1 (−104); chr20:24450704-24450819/SYNDIG1 (+927); chr20:
24854038-24854121/CST7 (−75786), SYNDIG1 (+404245); chr20:24929953-24930019/CST7 (+120); chr20:249
33797-24933921/CST7 (+3993), C20orf3 (+39566); chr20:25061789-25061978/VSX1 (+883); chr20:25062312-
25062510/VSX1 (+356); chr20:25062646-25062707/VSX1 (+90); chr20:25062818-25062870/VSX1 (−77); chr20:
25063907-25063993/VSX1 (−1183); chr20:25128881-25129058/VSX1 (−66203), ENTPD6 (−47369); chr20:25129
183-25129464/VSX1 (−66557), ENTPD6 (−47015); chr20:25371487-25371510/ABHD12 (+119); chr20:25566394-
25566411/NINL (−250); chr20:25604400-25604445/NANP (+225); chr20:25677583-25677622/ZNF337 (−134); c
hr20:25848703-25848718/ZNF337 (−171242); chr20:26188962-26189212/ZNF337 (−511618); chr20:26257594-
26257636/ZNF337 (−580146); chr20:29535006-29535084/DEFB115 (−310422); chr20:30102627-30102687/HM1
3 (+416); chr20:30249547-30249563/COX4I2 (+23864), BCL2L1 (+61101); chr20:30437621-30437640/FOXS1
(−4211); chr20:30458664-30458690/DUSP15 (−198), TTLL9 (+172); chr20:30946154-30946218/ASXL1 (+39); ch
r20:30946330-30946345/ASXL1 (+191); chr20:31062760-31062860/ASXL1 (+116663), C20orf203 (+176973); c
hr20:31070581-31070611/ASXL1 (+124449), C20orf203 (+169187); chr20:31097644-31097712/C20orf203 (+14
2105), ASXL1 (+151531); chr20:31116214-31116258/C20orf203 (+123547), ASXL1 (+170089); chr20:3116256
8-31162605/C20orf203 (+77196), ASXL1 (+216440); chr20:31168763-31168842/C20orf203 (+70980), ASXL1
(+222656); chr20:32031042-32031066/SNTA1 (+644); chr20:32255691-32255738/NECAB3 (+6549), CBFA2T2
(+177787); chr20:32400121-32400177/RALY (−181309), CHMP4B (+1039); chr20:32699820-32699881/EIF2S2
(+234); chr20:32803934-32803954/EIF2S2 (−103859), ASIP (−44227); chr20:32856888-32856951/ASIP (+8749),
AHCY (+34295); chr20:32951702-32951741/ITCH (+660); chr20:33064157-33064227/DYNLRB1 (−40012), ITC
H (+113130); chr20:33292376-33292469/TP53INP2 (+275); chr20:33813615-33813680/MMP24 (−891); chr20:3
4188632-34188747/FER1L4 (−15492), SPAG4 (−15119); chr20:34189089-34189166/FER1L4 (−15930), SPAG4
(−14681); chr20:34189492-34189679/FER1L4 (−16388), SPAG4 (−14223); chr20:34190131-34190145/FER1L4
(−16940), SPAG4 (−13671); chr20:34894429-34894563/DLGAP4 (−100952), EPB41L1 (+151834); chr20:3489474
3-34894784/DLGAP4 (−100684), EPB41L1 (+152102); chr20:34894907-34894954/DLGAP4 (−100517), EPB41L
1 (+152269); chr20:35089168-35089229/MYL9 (−80698), DLGAP4 (+93751); chr20:35169875-35169891/MYL9
(−14); chr20:35201585-35201647/TGIF2 (−260); chr20:35491100-35491258/DSN1 (−89025), SAMHD1 (+89067);
chr20:35579528-35579567/SAMHD1 (+698); chr20:35807829-35807869/C20orf132 (+125), RPN2 (+393); chr2
0:35974583-35974600/SRC (+35); chr20:36012804-36012910/NNAT (−136750), SRC (+38300); chr20:3601333
8-36013404/NNAT (−136236), SRC (+38814); chr20:36662484-36662527/TTI1 (−673); chr20:36889339-368893
74/TGM2 (−95657), BPI (−43195); chr20:37352516-37352606/SLC32A1 (−544); chr20:37353719-37353806/SLC
32A1 (+658); chr20:37354832-37354993/ACTR5 (−22184), SLC32A1 (+1808); chr20:37356043-37356168/ACT
R5 (−20991), SLC32A1 (+3001); chr20:37356828-37357215/ACTR5 (−20075), SLC32A1 (+3917); chr20:374343
53-37434488/PPP1R16B (+73); chr20:37434722-37434736/PPP1R16B (+381); chr20:37434894-37434930/PP
P1R16B (+564); chr20:37435370-37435487/FAM83D (−119526), PPP1R16B (+1081); chr20:37590350-3759041
9/DHX35 (−596); chr20:39311410-39311501/MAFB (+6420); chr20:39311813-39311890/MAFB (+6024); chr20:3
9312133-39312316/MAFB (+5651); chr20:39315625-39315685/MAFB (+2221); chr20:39316853-39316983/MA
FB (+958); chr20:39319204-39319514/MAFB (−1483); chr20:39968471-39968499/LPIN3 (−1075); chr20:399947
31-39994762/EMILIN3 (+751); chr20:39995911-39995970/EMILIN3 (−443); chr20:41817916-41818007/PTPRT
(+595); chr20:41818749-41818804/PTPRT (−220); chr20:42087042-42087060/SRSF6 (+547); chr20:42544534-4
2544727/TOX2 (+1139), JPH2 (+271587); chr20:42744647-42744693/JPH2 (+71548), TOX2 (+201178); chr20:
42744853-42744894/JPH2 (+71344), TOX2 (+201382); chr20:43160380-43160428/PKIG (−32); chr20:4327974
5-43279792/ADA (+607); chr20:43438086-43438334/RIMS4 (+769); chr20:43439219-43439247/RIMS4 (−254);
chr20:43540201-43540234/PABPC1L (+1515), TOMM34 (+48896); chr20:43726630-43726764/KCNS1 (+3056),
STK4 (+131577); chr20:43727118-43727132/KCNS1 (+2628), STK4 (+132005); chr20:43727254-43727272/K
CNS1 (+2490), STK4 (+132143); chr20:43729577-43729807/KCNS1 (+61); chr20:43921804-43921824/SLPI (−3
8608), RBPJL (−13677); chr20:43935459-43935498/RBPJL (−12); chr20:43945277-43945376/MATN4 (−8360), S
DC4 (+31737); chr20:44421342-44421366/WFDC3 (−807), DNTTIP1 (+778); chr20:44452579-44452665/TNNC2
(+3331), UBE2C (+11367); chr20:44639746-44639846/SLC12A5 (−10533), MMP9 (+2249); chr20:44640288-44
640312/SLC12A5 (−10029), MMP9 (+2753); chr20:44641134-44641192/SLC12A5 (−9166), MMP9 (+3616); chr2
0:44642369-44642404/SLC12A5 (−7942), MMP9 (+4840); chr20:44657840-44657944/SLC12A5 (+7563), NCOA
5 (+60688); chr20:44658302-44658361/SLC12A5 (+8003), NCOA5 (+60248); chr20:44686037-44686422/NCO
A5 (+32350), SLC12A5 (+35901); chr20:44686615-44686627/NCOA5 (+31959), SLC12A5 (+36292); chr20:446
87236-44687495/NCOA5 (+31214), SLC12A5 (+37037); chr20:44718363-44718419/NCOA5 (+189); chr20:447
28168-44728389/CD40 (−18627), NCOA5 (−9699); chr20:44803126-44803172/CD40 (+56243), CDH22 (+77183);
chr20:44879791-44880040/CDH22 (+416); chr20:44880167-44880275/CDH22 (+111); chr20:44936065-4493
6314/CDH22 (−55858), SLC35C2 (+56874); chr20:44936471-44936608/CDH22 (−56208), SLC35C2 (+56524); c
hr20:44937412-44937425/CDH22 (−57087), SLC35C2 (+55645); chr20:45034805-45034864/ELMO2 (+436); chr
20:45142039-45142151/ZNF334 (+99); chr20:45279982-45280039/SLC13A3 (+89); chr20:46356893-46356967/
SULF2 (+57878), NCOA3 (+226329); chr20:47443937-47443944/PREX1 (+479); chr20:47444428-47444452/P
REX1 (−20); chr20:47444581-47444609/PREX1 (−175); chr20:47444793-47444920/PREX1 (−437); chr20:47803
424-47803464/STAU1 (+1460), CSE1L (+140606); chr20:47836233-47836277/DDX27 (+423); chr20:48099294-
48099322/KCNB1 (−125); chr20:48184292-48184328/PTGIS (+397); chr20:48314715-48314807/PTGIS (−13005
4), B4GALT5 (+15660); chr20:48664925-48665027/SNAI1 (+65463), TMEM189-UBE2V1 (+105359); chr20:486
77258-48677314/SNAI1 (+77773), TMEM189-UBE2V1 (+93049); chr20:49127262-49127302/PTPN1 (+391); ch
r20:49307777-49307867/PARD6B (−40259), PTPN1 (+180931); chr20:49308020-49308045/PARD6B (−40048),
PTPN1 (+181142); chr20:49411898-49411929/BCAS4 (+447); chr20:49639185-49639217/KCNG1 (+474); chr2
0:49639857-49639882/KCNG1 (−195); chr20:49639997-49640094/KCNG1 (−371); chr20:49640348-49640409/K
CNG1 (−704); chr20:50109098-50109342/KCNG1 (−469545), NFATC2 (+50038); chr20:50418755-50418770/SA
LL4 (+285); chr20:50721201-50721234/SALL4 (−302170), ZFP64 (+87306); chr20:51588647-51588685/TSHZ2
(−280); chr20:52789949-52790081/CYP24A1 (+501); chr20:53092533-53092686/DOK5 (+344); chr20:53092868-
53092912/DOK5 (+624); chr20:54578974-54579139/CBLN4 (+955); chr20:54579959-54580069/CBLN4 (−2); c
hr20:54580522-54580621/CBLN4 (−560); chr20:54919226-54919287/FAM210B (−14726), MC3R (+95469); chr2
0:54934105-54934121/FAM210B (+130); chr20:55200311-55200615/TFAP2C (−3895); chr20:55201452-552015
80/TFAP2C (−2842); chr20:55202069-55202325/TFAP2C (−2161); chr20:55203438-55203505/TFAP2C (−886); c
hr20:55203669-55203806/TFAP2C (−620); chr20:55205789-55206293/TFAP2C (+1683), BMP7 (+635666); chr2
0:55206430-55206463/TFAP2C (+2089), BMP7 (+635260); chr20:55206598-55206736/TFAP2C (+2309), BMP
7 (+635040); chr20:55500347-55500405/TFAP2C (+296018), BMP7 (+341331); chr20:55500721-55501052/TF
AP2C (+296529), BMP7 (+340820); chr20:55532780-55532850/BMP7 (+308892), TFAP2C (+328457); chr20:5
5839668-55839692/BMP7 (+2027), TFAP2C (+635322); chr20:55840246-55840279/BMP7 (+1444), TFAP2C
(+635905); chr20:55841990-55842055/BMP7 (−316); chr20:55964916-55964997/RBM38 (−1497); chr20:55965169-
55965308/RBM38 (−1215); chr20:55965703-55965803/RBM38 (−701); chr20:56056599-56056674/CTCFL (+43
546), RBM38 (+90183); chr20:56196092-561 96223/ZBP1 (−526); chr20:56725875-56725919/PMEPA1 (−440866),
PPP4R1L (+158598); chr20:56732732-56732867/PMEPA1 (−447769), PPP4R1L (+151695); chr20:56785320-
56785406/PMEPA1 (−500332), PPP4R1L (+99132); chr20:56964032-56964080/VAPB (−119); chr20:57089460-
57090103/APCDD1L (+167); chr20:57224597-57224744/STX16 (−1638); chr20:57225080-57225218/STX16 (−1
160); chr20:57281787-57281821/GNAS (−132991), NPEPL1 (+13942); chr20:581 52372-581 52388/PHACTR3
(−27223), EDN3 (+276881); chr20:58152559-58152775/PHACTR3 (−26936), EDN3 (+277168); chr20:58179995-5
8180213/PHACTR3 (+501); chr20:58180403-58180470/PHACTR3 (+834); chr20:58180576-58180687/PHACTR
3 (+1029), SYCP2 (+326577); chr20:58394704-58394804/SYCP2 (+112455), PHACTR3 (+215151); chr20:5863
1000-58631143/CDH26 (+97601); chr20:58662536-58662587/CDH26 (+129091); chr20:58662841-58662926/C
DH26 (+129413); chr20:59053194-59053253/CDH4 (−774258), CDH26 (+519753); chr20:59827143-59827237/
CDH4 (−292); chr20:59827408-59827417/CDH4 (−69); chr20:59827764-59827920/CDH4 (+360); chr20:5982816
6-59828235/CDH4 (+719); chr20:59828408-59828804/CDH4 (+1124), TAF4 (+812260); chr20:59829213-59829
284/CDH4 (+1767), TAF4 (+811617); chr20:59868948-59869042/CDH4 (+41513), TAF4 (+771871); chr20:6054
0425-60540485/TAF4 (+100411), CDH4 (+712973); chr20:60640796-60640883/TAF4 (+26); chr20:60641030-6
0641047/TAF4 (−173); chr20:60962626-60962687/RPS21 (+536); chr20:60981908-60981936/CABLES2 (+417);
chr20:61008830-61008914/CABLES2 (−26533), GATA5 (+42154); chr20:61049934-61050222/GATA5 (+948); c
hr20:61050351-61050418/GATA5 (+641); chr20:61051176-61051250/GATA5 (−187); chr20:61051437-6105146
9/GATA5 (−427); chr20:61051855-61051879/GATA5 (−841); chr20:61087967-61088125/SLCO4A1 (−185751), G
ATA5 (−37020); chr20:61159233-61159299/SLCO4A1 (−114531), GATA5 (−108240); chr20:61359948-61359978/
C20orf20 (−67842), NTSR1 (+19774); chr20:61493016-61493113/TCFL5 (+50); chr20:61493511-61493534/TC
FL5 (−408); chr20:61557000-61557065/DIDO1 (+870); chr20:61560461-61560528/DIDO1 (−2592); chr20:61569
108-61569156/C20orf11 (−309); chr20:61585923-61585989/SLC17A9 (+1957), BHLHE23 (+52431); chr20:6160
4758-61604777/SLC17A9 (+20769), BHLHE23 (+33619); chr20:61610210-61610287/SLC17A9 (+26250), BHL
HE23 (+28138); chr20:61612348-61612479/BHLHE23 (+25973), SLC17A9 (+28415); chr20:61636335-6163638
1/BHLHE23 (+2029), SLC17A9 (+52359); chr20:61636779-61636875/BHLHE23 (+1560), SLC17A9 (+52828); c
hr20:61637091-61637281/BHLHE23 (+1201), SLC17A9 (+53187); chr20:61637649-61637735/BHLHE23 (+695);
chr20:61637957-61638220/BHLHE23 (+298); chr20:61638470-61638534/BHLHE23 (−115); chr20:61638798-6
1639020/BHLHE23 (−522); chr20:61703466-61703642/BIRC7 (−163722), BHLHE23 (−65167); chr20:61703762-6
1703845/BIRC7 (−163472), BHLHE23 (−65417); chr20:61734088-61734101/BIRC7 (−133181), BHLHE23 (−9570
8); chr20:61808381-61808666/BHLHE23 (−170137), BIRC7 (−58752); chr20:61809006-61809218/BHLHE23 (−17
0725), BIRC7 (−58164); chr20:61809632-61809840/BHLHE23 (−171349), BIRC7 (−57540); chr20:61810135-618
10159/BHLHE23 (−171760), BIRC7 (−57129); chr20:61810557-61810632/BHLHE23 (−172208), BIRC7 (−56681);
chr20:61885103-61885195/NKAIN4 (+743); chr20:61885551-61885777/NKAIN4 (+228); chr20:61886204-61886
256/NKAIN4 (−338); chr20:61992271-61992532/CHRNA4 (+293); chr20:61992636-61992674/CHRNA4 (+40); c
hr20:62102664-62102699/CHRNA4 (−109987), KCNQ2 (+1311); chr20:62103626-62103667/KCNQ2 (+346); chr
20:62131025-62131120/EEF1A2 (−568); chr20:62152341-62152366/PPDPF (+221); chr20:62198818-62198930/
SRMS (−20017), PRIC285 (+6718); chr20:62199538-62199644/SRMS (−20734), PRIC285 (+6001); chr20:6220
5754-62205804/PRIC285 (−187); chr20:62259221-62259259/GMEB2 (−859); chr20:62272683-62272797/GMEB
2 (−14359), STMN3 (+12040); chr20:62338742-62338766/ZGPAT (−626), ARFRP1 (+601); chr20:62339118-623
39163/ZGPAT (−239), ARFRP1 (+214); chr20:62369728-62369771/SLC2A4RG (−1461); chr20:62525611-62525
692/DNAJC5 (−803); chr20:62572980-62573066/UCKL1 (+14777), DNAJC5 (+46568); chr20:62573214-625733
44/UCKL1 (+14521), DNAJC5 (+46824); chr20:62579648-62579705/UCKL1 (+8123), DNAJC5 (+53222); chr20:
62673334-62673410/SOX18 (+7607), PRPF6 (+60941); chr20:62680231-62680421/SOX18 (+653); chr20:6269
4426-62694477/TCEA2 (+6013), RGS19 (+16872); chr20:62696907-62696956/TCEA2 (+8493), RGS19 (+1439
2); chr20:62734130-62734172/NPBWR2 (+4033), OPRL1 (+17782); chr20:62947985-62948007/PCMTD2 (+609
48); chr20:62959203-62959409/PCMTD2 (+72258); chr21:9664665-9664710/TEKT4P2 (+303905); chr21:1961
7111-19617175/CHODL (−7); chr21:19617276-19617418/CHODL (+197); chr21:19617966-19618097/CHODL
(+882); chr21:22369362-22369429/NCAM2 (−1237); chr21:22370225-22370236/NCAM2 (−402); chr21:22370476-
22370552/NCAM2 (−119); chr21:27012121-27012148/JAM2 (+546); chr21:27945148-27945252/CYYR1 (+381);
chr21:27945436-27945604/CYYR1 (+61); chr21:28216583-28216633/CYYR1 (−271027), ADAMTS1 (+1120); ch
r21:28337916-28338026/ADAMTS1 (−120243), ADAMTS5 (+1468); chr21:28338336-28338515/ADAMTS1 (−12
0698), ADAMTS5 (+1013); chr21:28340049-28340062/ADAMTS5 (−617); chr21:30445418-30445475/CCT8 (+5
63); chr21:31311312-31311386/GRIK1 (+933); chr21:31311634-31311700/GRIK1 (+615); chr21:31311920-313
12079/GRIK1 (+282); chr21:32546244-32546314/KRTAP19-8 (−135484), TIAM1 (+385011); chr21:33984233-33
984280/C21orf59 (+661); chr21:34100323-34100461/SYNJ1 (−41); chr21:34392168-34392205/C21orf62 (−2061
34), OLIG2 (−6029); chr21:34392404-34392436/C21orf62 (−206367), OLIG2 (−5796); chr21:34399259-34399441/
OLIG1 (−43100), OLIG2 (+1134); chr21:34400105-34400231/OLIG1 (−42282), OLIG2 (+1952); chr21:34444363-
34444444/IFNAR2 (−157827), OLIG1 (+1954); chr21:34602246-34602267/IFNAR2 (+26); chr21:34602738-346
02763/IFNAR2 (+520); chr21:34852117-34852164/DNAJC28 (+11882), IFNGR2 (+76939); chr21:34960582-349
60632/DONSON (+660); chr21:35446551-35446589/MRPS6 (+747), SLC5A3 (+747); chr21:35575010-3557506
4/KCNE2 (−161286), MRPS6 (+129214); chr21:35747581-35747645/FAM165B (−136); chr21:35831912-358320
03/KCNE1 (−3895); chr21:36041360-36041700/CLIC6 (−158); chr21:36042029-36042091/CLIC6 (+372); chr21:3
6419174-36419236/RUNX1-IT1 (−7482), RUNX1 (+2390); chr21:36900927-36900988/CBR1 (−541327), RUNX1
(−479363); chr21:37502795-37502824/CBR3 (−4453); chr21:38064113-38064248/CLDN14 (−225456), SIM2 (−78
10); chr21:38065523-38065799/CLDN14 (−226936), SIM2 (−6330); chr21:38068230-38068646/SIM2 (−3553); ch
r21:38069806-38069853/SIM2 (−2161); chr21:38070217-38070412/SIM2 (−1676); chr21:38073170-38073239/SI
M2 (+1214), HLCS (+280059); chr21:38073526-38073615/SIM2 (+1580), HLCS (+279693); chr21:38076828-38
077129/SIM2 (+4988), HLCS (+276285); chr21:38077431-38077617/SIM2 (+5533), HLCS (+275740); chr21:38
080063-38080174/SIM2 (+8128), HLCS (+273145); chr21:38080407-38080471/SIM2 (+8448), HLCS (+272825);
chr21:38081340-38081536/SIM2 (+9447), HLCS (+271826); chr21:38082640-38082684/SIM2 (+10671), HLC
S (+270602); chr21:38120538-38120553/SIM2 (+48555), HLCS (+232718); chr21:38338608-38338650/HLCS
(+14635), SIM2 (+266638); chr21:38362391-38362412/DSCR6 (−16461), HLCS (−9138); chr21:38378336-38378
464/DSCR6 (−463); chr21:38445715-38445740/PIGP (−625), TTC3 (+157); chr21:38639559-38639615/DSCR3
(+246); chr21:38640142-38640190/DSCR3 (−333); chr21:39288704-39288790/KCNJ6 (−51); chr21:40032321-40
032367/ERG (−161916), ETS2 (−145505); chr21:40105944-40105997/ERG (−235543), ETS2 (−71878); chr21:40
684774-40684842/BRWD1 (+904); chr21:42218515-42218595/DSCAM (+484); chr21:42218931-42218980/DS
CAM (+83); chr21:42539543-42539583/BACE2 (−165); chr21:42880225-42880260/TMPRSS2 (−251); chr21:431
46051-43146129/TMPRSS2 (−266098), RIPK4 (+41159); chr21:43181496-43181626/TMPRSS2 (−301569), RIP
K4 (+5688); chr21:43183619-43183721/TMPRSS2 (−303678), RIPK4 (+3579); chr21:43186434-43186592/RIPK
4 (+736); chr21:43429287-43429338/C2CD2 (−55314), ZNF295 (+1183); chr21:43916158-43916199/SLC37A1
(−3563), RSPH1 (+222); chr21:43953090-43953146/PDE9A (−120744), SLC37A1 (+33376); chr21:44104429-441
04524/PDE9A (+30615), WDR4 (+195201); chr21:44164911-44164949/PDE9A (+91068), WDR4 (+134748); chr
21:44259714-44259802/WDR4 (+39920), PDE9A (+185896); chr21:44494778-44494940/CBS (+1613), PKNOX
1 (+100216); chr21:44496970-44496987/CBS (−507); chr21:44527528-44527576/U2AF1 (+136); chr21:4478229
6-44782503/SIK1 (+64602), CRYAA (+193259); chr21:44803650-44803685/SIK1 (+43334), CRYAA (+214527);
chr21:44819111-44819309/SIK1 (+27792), CRYAA (+230069); chr21:44819972-44820075/SIK1 (+26978), CRY
AA (+230883); chr21:44846340-44846380/SIK1 (+642); chr21:45149027-45149198/PDXK (+10135), CSTB (+4
7146); chr21:45237759-45237827/AGPAT3 (−47323), RRP1 (+28375); chr21:45246277-45246324/AGPAT3 (−3
8815), RRP1 (+36883); chr21:45563266-45563294/C21orf33 (+9786), ICOSLG (+97554); chr21:45577552-455
77591/C21orf33 (+24078), ICOSLG (+83262); chr21:45577752-45577842/C21orf33 (+24303), ICOSLG (+83037);
chr21:45592692-45592949/C21orf33 (+39327), ICOSLG (+68013); chr21:45627276-45627371/ICOSLG (+33
510), C21orf33 (+73830); chr21:45770272-45770304/TRPM2 (−3196); chr21:45770666-45770688/TRPM2 (−280
7); chr21:45924177-45924274/KRTAP10-1 (+35852), LRRC3 (+48833); chr21:46048145-46048203/KRTAP10-1
0 (−9099), KRTAP10-9 (+1134); chr21:46126428-46126566/TSPEAR (+4998), KRTAP10-12 (+9410); chr21:462
92671-46292743/SUMO3 (−54663), PTTG1IP (+1111); chr21:46331566-46331662/PTTG1IP (−37796), ITGB2
(+9351); chr21:46334373-46334459/PTTG1IP (−40598), ITGB2 (+6549); chr21:46360142-46360167/C21orf67 (−3
27); chr21:46438972-46439007/C21orf67 (−79162), ADARB1 (−55503); chr21:46706975-46707016/POFUT2 (+8
15); chr21:46824246-46824277/POFUT2 (−116451), COL18A1 (−51162); chr21:46897110-46897232/COL18A1
(+21747), SLC19A1 (+65214); chr21:46902889-46902922/COL18A1 (+27482), SLC19A1 (+59479); chr21:46955
460-46955496/SLC19A1 (+6907), COL18A1 (+80054); chr21:46962289-46962342/SLC19A1 (+69); chr21:4696
2899-46962915/SLC19A1 (−522); chr21:46975642-46975668/PCBP3 (−294220), SLC19A1 (−13270); chr21:4701
0230-47010262/PCBP3 (−259629), SLC19A1 (−47861); chr21:47062156-47062175/PCBP3 (−207709), SLC19A1
(−99781); chr21:47062367-47062442/PCBP3 (−207470), SLC19A1 (−100020); chr21:47275485-47275610/COL6
A1 (−126115), PCBP3 (+5673); chr21:47392659-47392726/COL6A1 (−8970), PCBP3 (+122818); chr21:4741073
0-47410793/COL6A2 (−107271), COL6A1 (+9099); chr21:47518313-47518349/COL6A2 (+298); chr21:4751886
6-47518887/COL6A2 (+844); chr21:47705695-47705732/YBEY (−553), MCM3AP (−478); chr21:47717387-4771
7433/PCNT (−26626), YBEY (+11143); chr21:47717673-47717748/PCNT (−26325), YBEY (+11444); chr21:4771
7952-47717981/PCNT (−26069), YBEY (+11700); chr21:47878720-47878745/DIP2A (−129); chr21:47971700-47
971831/S100B (+53269), DIP2A (+92904); chr21:48055801-48055968/PRMT2 (+378); chr22:17001401-170014
09/OR11H1 (−551601), CCT8L2 (+72295); chr22:17001535-17001595/OR11H1 (−551761), CCT8L2 (+72135); c
hr22:17081936-17082068/CCT8L2 (−8302), XKR3 (+220582); chr22:17082532-17082758/CCT8L2 (−8945), XKR
3 (+219939); chr22:17082894-17082988/CCT8L2 (−9241), XKR3 (+219643); chr22:17083411-17083524/CCT8L
2 (−9768), XKR3 (+219116); chr22:17083774-17083820/CCT8L2 (−10097), XKR3 (+218787); chr22:17084068-1
7084117/CCT8L2 (−10393), XKR3 (+218491); chr22:17090664-17090840/CCT8L2 (−17052), XKR3 (+211832); c
hr22:17091010-17091036/CCT8L2 (−17323), XKR3 (+211561); chr22:17567050-17567093/IL17RA (+1221), CE
CR5 (+73097); chr22:17601134-17601259/IL17RA (+35346), CECR5 (+38972); chr22:17628264-17628291/CE
CR5 (+11891), IL17RA (+62427); chr22:17652593-17652654/CECR5 (−12455), CECR1 (+38155); chr22:178494
27-17849501/CECR1 (−158685), CECR2 (−107166); chr22:18594073-18594113/TUBA8 (+640); chr22:1863281
0-18632850/USP18 (+72); chr22:18893854-18893877/DGCR6 (+130); chr22:18923603-18923626/PRODH (+45
1); chr22:18958356-18958403/PRODH (−34314), DGCR2 (+151587); chr22:19109138-19109201/DGCR2 (+797);
chr22:19136325-19136557/DGCR14 (−4251); chr22:19136696-19136720/DGCR14 (−4518); chr22:19138322-
19138341/GSC2 (−536); chr22:19138632-19138682/GSC2 (−861); chr22:19419201-19419255/MRPL40 (−808),
HIRA (−9); chr22:19467659-19467702/UFD1L (−943), CDC45 (+267); chr22:19510947-19511141/CLDN5 (+1816),
CDC45 (+43630); chr22:19511456-19511541/CLDN5 (+1361), CDC45 (+44085); chr22:19512005-19512083/
CLDN5 (+816); chr22:19512319-19512376/CLDN5 (+512); chr22:19683303-19683379/CLDN5 (−170481), SEPT
5 (−18646); chr22:19710616-19710698/TBX1 (−33569), GP1BB (+4665); chr22:19711326-19711609/TBX1 (−327
58), GP1BB (+5476); chr22:19711740-19711891/TBX1 (−32410), GP1BB (+5824); chr22:19742758-19742788/T
BX1 (−1453); chr22:19743335-19743352/TBX1 (−882); chr22:19743967-19744034/TBX1 (−225); chr22:1974610
7-19746163/TBX1 (+1909), GNB1L (+96327); chr22:19747011-19747099/TBX1 (+2829), GNB1L (+95407); chr
22:19748909-19748960/TBX1 (+4709), GNB1L (+93527); chr22:19749069-19749084/TBX1 (+4851), GNB1L
(+93385); chr22:19753378-19753516/TBX1 (+9221), GNB1L (+89015); chr22:19753687-19753869/TBX1 (+9552),
GNB1L (+88684); chr22:19754819-19754837/TBX1 (+10602), GNB1L (+87634); chr22:19760403-19760532/T
BX1 (+16242), GNB1L (+81994); chr22:20104374-20104413/RANBP1 (−630), TRMT2A (+374); chr22:20105204-
20105250/TRMT2A (−459), RANBP1 (+203); chr22:20234524-20234617/RTN4R (+21245), ZDHHC8 (+115207);
chr22:20255181-20255234/RTN4R (+608); chr22:20255670-20255700/RTN4R (+131); chr22:20255880-20255
907/RTN4R (−78); chr22:20380069-20380138/TMEM191B (+2435), USP41 (+351438); chr22:20484233-204842
59/TMEM191B (+106577), USP41 (+247296); chr22:20779799-20779941/SCARF2 (+12276), ZNF74 (+31390);
chr22:20780407-20780437/SCARF2 (+11724), ZNF74 (+31942); chr22:20783549-20783647/SCARF2 (+8548),
ZNF74 (+35118); chr22:20784124-20784192/SCARF2 (+7988), ZNF74 (+35678); chr22:20784980-20785060/S
CARF2 (+7126), ZNF74 (+36540); chr22:20785372-20785608/SCARF2 (+6656), ZNF74 (+37010); chr22:20785
897-20786069/SCARF2 (+6163), ZNF74 (+37503); chr22:20792370-20792481/SCARF2 (−280); chr22:2086223
3-20862291/MED15 (+376); chr22:21025335-21025538/SERPIND1 (−102946), MED15 (+163551); chr22:21212
833-21212884/SNAP29 (−433), PI4KA (+241); chr22:21272199-21272239/CRKL (+505); chr22:21355987-21356
050/THAP7 (+385); chr22:21368382-21368435/P2RX6 (−1055); chr22:21386877-21386893/SLC7A4 (−38); chr2
2:21714756-21714880/GGT2 (−151046), HIC2 (−56875); chr22:21748474-21748600/GGT2 (−184765), HIC2 (−23
156); chr22:21774713-21774820/TMEM191C (−46692), HIC2 (+3074); chr22:21823888-21823967/TMEM191C
(+2469), PI4KAP2 (+22455); chr22:22090528-22090836/YPEL1 (−611); chr22:22472319-22472376/TOP3B (−135
201), VPREB1 (−126852); chr22:23413253-23413295/GNAZ (+605); chr22:23524135-23524303/BCR (+1667), I
GLL1 (+398276); chr22:23863737-23863792/IGLL1 (+58730), BCR (+341213); chr22:23864046-23864080/IGL
L1 (+58432), BCR (+341511); chr22:23864871-23864921/IGLL1 (+57599), BCR (+342344); chr22:24109695-24
109731/CHCHD10 (+428); chr22:24109915-24109955/CHCHD10 (+206); chr22:24129201-24129236/SMARCB
1 (+69); chr22:24180994-24181054/DERL3 (+175); chr22:24890963-24890979/UPB1 (+894); chr22:24951586-
24951655/SNRPD3 (+3); chr22:25081894-25081993/PIWIL3 (+88739), GGT1 (+102226); chr22:25466115-254
66179/TMEM211 (−130833), CRYBB3 (−129678); chr22:25507818-25507934/TMEM211 (−172562), CRYBB3 (−8
7949); chr22:25678869-25679042/CRYBB2 (+63344), LRP5L (+98588); chr22:25679250-25679267/CRYBB2
(+63647), LRP5L (+98285); chr22:25800852-25800898/ADRBK2 (−159986), LRP5L (−23331); chr22:25961081-25
961110/ADRBK2 (+235); chr22:25961462-25961536/ADRBK2 (+638); chr22:26565361-26565412/SEZ6L (−53);
chr22:26565651-26565734/SEZ6L (+253); chr22:26908246-26908292/TFIP11 (+168); chr22:26966160-269662
40/TFIP11 (−57763), TPST2 (+19889); chr22:27299726-27299779/CRYBA4 (+281825), MN1 (+897733); chr22:
27520524-27520593/CRYBA4 (+502631), MN1 (+676927); chr22:27719837-27719868/MN1 (+477633), CRYBA
4 (+701925); chr22:28028976-28029062/MN1 (+168467); chr22:28315336-28315374/PITPNB (−100); chr22:283
15562-28315626/PITPNB (−339); chr22:29075313-29075343/TTC28 (+525); chr22:29138266-29138322/CHEK2
(−472), HSCB (+251); chr22:29169014-29169078/XBP1 (+27514), HSCB (+31003); chr22:29469173-29469213/
KREMEN1 (+127); chr22:29469415-29469460/KREMEN1 (+372); chr22:29469633-29469664/KREMEN1 (+583);
chr22:29664509-29664534/RHBDD3 (−608), EWSR1 (+524); chr22:29709633-29709735/RASL10A (+2064),
GAS2L1 (+6687); chr22:29875975-29876004/NEFH (−191); chr22:29876295-29876396/NEFH (+165); chr22:30
117147-30117268/CABP7 (+864); chr22:30234371-30234397/ASCC2 (−91); chr22:30476148-30476196/HORM
AD2 (−281); chr22:30476321-30476413/HORMAD2 (−86); chr22:30642115-30642171/LIF (+653); chr22:306619
99-30662076/OSM (+791); chr22:30662703-30662798/OSM (+78); chr22:30667088-30667110/OSM (−4270); ch
r22:30674032-30674178/OSM (−11276), TBC1D10A (+48850); chr22:30675510-30675606/OSM (−12729), TBC
1D10A (+47397); chr22:30820372-30820431/SEC14L2 (+27472), SEC14L3 (+47632); chr22:30938507-309385
42/SEC14L4 (−36827), SEC14L6 (+4144); chr22:31063822-31063850/DUSP18 (+36); chr22:31364578-3136465
4/MORC2 (−429); chr22:31364775-31364862/MORC2 (−632); chr22:32058304-32058361/PISD (−31523), PRR1
4L (+87787); chr22:32340089-32340155/YWHAH (−357); chr22:32596799-32596917/RFPL2 (+2606), SLC5A1
(+157839); chr22:32870851-32870893/FBXO7 (+165); chr22:33271249-33271345/TIMP3 (+74495), SYN3 (+131
512); chr22:33454075-33454193/SYN3 (−51325), LARGE (+862282); chr22:35696542-35696625/TOM1 (+787);
chr22:35748604-35748696/HMOX1 (−28410), TOM1 (+52853); chr22:35768313-35768357/HMOX1 (−8725), TO
M1 (+72538); chr22:35877018-35877087/RASD2 (−60299), MCM5 (+80937); chr22:36424395-36424418/RBFO
X2 (+178); chr22:36784011-36784043/MYH9 (+36); chr22:36806000-36806060/MYH9 (−21967), TXN2 (+71657);
chr22:37212774-37212813/IFT27 (−40622), PVALB (+2723); chr22:37476126-37476160/TMPRSS6 (+23550),
KCTD17 (+28364); chr22:37637654-37637726/SSTR3 (−29337), RAC2 (+2615); chr22:37641356-37641422/R
AC2 (−1084); chr22:37730870-37730926/CYTH4 (+52403), ELFN2 (+92607); chr22:37731366-37731458/CYTH
4 (+52917), ELFN2 (+92093); chr22:37815754-37815811/ELFN2 (+7722), CYTH4 (+137288); chr22:37816463-
37816501/ELFN2 (+7023), CYTH4 (+137987); chr22:37880253-37880316/ELFN2 (−56780), MFNG (+2193); chr
22:37881146-37881430/ELFN2 (−57783), MFNG (+1190); chr22:37881686-37881787/MFNG (+741); chr22:378
82083-37882195/MFNG (+339); chr22:37957203-37957264/CDC42EP1 (+763); chr22:37962021-37962049/CD
C42EP1 (+5564), LGALS2 (+13989); chr22:37962537-37962581/CDC42EP1 (+6088), LGALS2 (+13465); chr22:
38069084-38069118/LGALS1 (−2512); chr22:38142681-38142778/H1F0 (−58384), TRIOBP (+49735); chr22:38
201231-38201269/GCAT (−2662), H1F0 (+136); chr22:38203613-38203676/GCAT (−267); chr22:38477132-384
77296/SLC16A8 (+1956), PICK1 (+23952); chr22:38478476-38478852/SLC16A8 (+506); chr22:38484991-3848
5056/SLC16A8 (−5854), BAIAP2L2 (+21652); chr22:38572663-38572759/BAIAP2L2 (−66035), PLA2G6 (+5050);
chr22:38793981-38794013/CSNK1E (−79908), KCNJ4 (+46036); chr22:38901857-38901888/DDX17 (+472); ch
r22:39102386-39102404/GTPBP1 (+588); chr22:39268356-39268385/CBX6 (−113); chr22:39540122-39540237/
CBX7 (+8358), APOBEC3H (+46951); chr22:39631410-39631517/CBX7 (−82926), PDGFB (+9493); chr22:3963
1785-39631826/CBX7 (−83268), PDGFB (+9151); chr22:39703024-39703155/PDGFB (−62133), RPL3 (+12580);
chr22:39853657-39853690/MGAT3 (−29555), TAB1 (+57915); chr22:39898153-39898201/SMCR7L (−107); chr
22:39898624-39898684/SMCR7L (+370); chr22:39953800-39953818/RPS19BP1 (−24949), CACNA1I (−12949);
chr22:39954007-39954043/RPS19BP1 (−25165), CACNA1I (−12733); chr22:39954400-39954428/RPS19BP1 (−2
5554), CACNA1I (−12344); chr22:40013624-40013703/GRAP2 (−283422), CACNA1I (+46906); chr22:40058029-
40058103/GRAP2 (−239020), CACNA1I (+91308); chr22:40058370-40058412/GRAP2 (−238695), CACNA1I (+9
1633); chr22:40060766-40060841/GRAP2 (−236282), CACNA1I (+94046); chr22:40081419-40081454/GRAP2
(−215649), CACNA1I (+114679); chr22:40081776-40081791/GRAP2 (−215302), CACNA1I (+115026); chr22:400
82129-40082159/GRAP2 (−214942), CACNA1I (+115386); chr22:40082263-40082282/GRAP2 (−214813), CAC
NA1I (+115515); chr22:41032376-41032405/MKL1 (+299); chr22:41593569-41593634/L3MBTL2 (−7711), EP30
0 (+104988); chr22:41864395-41864430/ACO2 (−716), PHF5A (+295); chr22:41864672-41864702/ACO2 (−442),
PHF5A (+21); chr22:42307040-42307095/SHISA8 (+3603), SREBF2 (+77962); chr22:42316139-42316291/SHI
SA8 (−5544), TNFRSF13C (+6606); chr22:42372574-42372621/SEPT3 (−333); chr22:42373398-42373458/SEP
T3 (+497); chr22:42466273-42466344/FAM109B (−3946), NAGA (+537); chr22:42466528-42466598/FAM109B
(−3692), NAGA (+283); chr22:42470035-42470060/NAGA (−3202), FAM109B (−207); chr22:42736919-42737016/
TCF20 (−125523), NFAM1 (+91433); chr22:42737245-42737293/TCF20 (−125824), NFAM1 (+91132); chr22:428
28275-42828385/NFAM1 (+71); chr22:42929062-42929133/SERHL2 (−20827), RRP7A (−13269); chr22:430581
82-43058216/CYB5R3 (−17559), A4GALT (+58677); chr22:43288234-43288361/ARFGAP3 (−34890), PACSIN2
(+122886); chr22:43410512-43410539/PACSIN2 (+658); chr22:43506692-43506719/BIK (−48); chr22:43524258-
43524355/MCAT (+15096), BIK (+17553); chr22:43582442-43582518/TTLL12 (+657); chr22:43582691-435827
38/TTLL12 (+422); chr22:43659516-43659545/TTLL12 (−76394), SCUBE1 (+79863); chr22:43738706-4373874
3/SCUBE1 (+669); chr22:43738900-43738928/SCUBE1 (+480); chr22:43739519-43739548/SCUBE1 (−140); ch
r22:43739781-43739825/SCUBE1 (−409); chr22:43807517-43807547/MPPED1 (−488); chr22:44258042-442580
78/SULT4A1 (+318); chr22:44319835-44319869/PNPLA3 (+233); chr22:44464852-44464991/PARVG (−112335),
PARVB (+44765); chr22:44726978-44727058/PRR5-ARHGAP8 (−371354), KIAA1644 (−18287); chr22:450544
02-45054467/KIAA1644 (−345704), PRR5-ARHGAP8 (−43937); chr22:45064237-45064286/KIAA1644 (−355531),
PRR5-ARHGAP8 (−34110); chr22:45125565-45125610/PRR5-ARHGAP8 (+27216), PHF21B (+280221); chr22:
45236685-45236697/PRR5-ARHGAP8 (+138319), PHF21B (+169118); chr22:45405011-45405046/PHF21B
(+780); chr22:45405432-45405476/PHF21B (+355); chr22:45405959-45406039/PHF21B (−190); chr22:45665089-
45665122/UPK3A (−15762), NUP50 (+105380); chr22:45899185-45899227/FBLN1 (+487); chr22:46262307-462
62367/WNT7B (+110671), ATXN10 (+194659); chr22:46263052-46263123/WNT7B (+109920), ATXN10 (+1954
10); chr22:46263297-46263511/WNT7B (+109604), ATXN10 (+195726); chr22:46263624-46263743/WNT7B
(+109324), ATXN10 (+196006); chr22:46264633-46264689/WNT7B (+108347), ATXN10 (+196983); chr22:46276
386-46276442/WNT7B (+96594), ATXN10 (+208736); chr22:46307952-46308014/WNT7B (+65025), ATXN10
(+240305); chr22:46362349-46362429/WNT7B (+10619), ATXN10 (+294711); chr22:46366288-46366507/WNT
7B (+6610), ATXN10 (+298720); chr22:46367360-46367532/WNT7B (+5562), ATXN10 (+299768); chr22:46367
987-46368102/WNT7B (+4963), ATXN10 (+300367); chr22:46368256-46368656/WNT7B (+4552), ATXN10 (+3
00778); chr22:46372761-46372769/WNT7B (+243); chr22:46441099-46441131/PPARA (−105384), WNT7B (−68
107); chr22:46449549-46449563/PPARA (−96943), WNT7B (−76548); chr22:46466564-46466611/WNT7B (−935
80), PPARA (−79911); chr22:46546560-46546609/PPARA (+86); chr22:46658904-46658938/TTC38 (−4940), PK
DREJ (+298); chr22:46684197-46684336/GTSE1 (−8371), TTC38 (+20406); chr22:46693149-46693205/GTSE1
(+539); chr22:46861455-46861485/CELSR1 (+71597), TRMU (+130172); chr22:46932504-46932533/CELSR1
(+548); chr22:47070140-47070190/GRAMD4 (+47517), CERK (+63987); chr22:47070594-47070719/GRAMD4
(+48009), CERK (+63495); chr22:47081750-47081788/CERK (+52383), GRAMD4 (+59121); chr22:47170624-47
170666/TBC1D22A (+12127); chr22:47199208-47199299/TBC1D22A (+40736); chr22:47798173-47798287/TB
C1D22A (+639712); chr22:48884897-48884959/FAM19A5 (−360); chr22:48885842-48885886/FAM19A5 (+576);
chr22:48962717-48962779/FAM19A5 (+77460); chr22:48972466-48972486/FAM19A5 (+87188); chr22:489726
04-48972617/FAM19A5 (+87323); chr22:48976420-48976468/FAM19A5 (+91156); chr22:49098636-49098700/
FAM19A5 (+213380); chr22:49856630-49856682/BRD1 (+361796), FAM19A5 (+971368); chr22:50050839-500
50857/BRD1 (+167604); chr22:50064662-50064759/BRD1 (+153741); chr22:50118697-50118722/BRD1 (+997
42); chr22:50120592-50120642/BRD1 (+97835); chr22:50201726-50201802/BRD1 (+16688); chr22:50220453-
50220504/BRD1 (−2027); chr22:50241846-50241887/BRD1 (−23415), ZBED4 (−5630); chr22:50242156-502421
90/BRD1 (−23721), ZBED4 (−5324); chr22:50243523-50243630/ZBED4 (−3920); chr22:50312481-50312502/AL
G12 (−386), CRELD2 (+209); chr22:50354085-50354104/PIM3 (−48); chr22:50422226-50422427/PIM3 (+68184),
MLC1 (+101454); chr22:50438273-50438305/PIM3 (+84146), MLC1 (+85492); chr22:50497183-50497213/ML
C1 (+26583), PIM3 (+143055); chr22:50551101-50551226/PANX2 (−57996), MOV10L1 (+22729); chr22:506233
35-50623414/PANX2 (+14215), TUBGCP6 (+60025); chr22:50623527-50623536/PANX2 (+14372), TUBGCP6
(+59868); chr22:50623715-50623741/PANX2 (+14568), TUBGCP6 (+59672); chr22:50640368-50640412/PANX
2 (+31230), TUBGCP6 (+43010); chr22:50709292-50709348/MAPK11 (−541); chr22:50731831-50731883/MAP
K11 (−23078), PLXNB2 (+14144); chr22:50732030-50732079/MAPK11 (−23276), PLXNB2 (+13946); chr22:5075
2736-50752783/PPP6R2 (−28986), PLXNB2 (−6759); chr22:50945836-50945940/NCAPH2 (−757), LMF2 (+247);
chr22:50946098-50946119/NCAPH2 (−536), LMF2 (+26); chr22:50946720-50946784/LMF2 (−617), NCAPH2
(+107); chr22:50946962-50946998/LMF2 (−845), NCAPH2 (+335); chr22:50986971-50987013/ODF3B (−15984), C
PT1B (+29902); chr22:50987302-50987509/ODF3B (−16398), CPT1B (+29488); chr22:51021410-51021437/CP
T1B (−4530), CHKB (+4); chr22:51042030-51042182/MAPK8IP2 (+544); chr22:51042405-51042457/MAPK8IP2
(+869); chr22:51042566-51042806/MAPK8IP2 (+1124), ARSA (+23915); chr22:51042935-51042973/MAPK8IP2
(+1392), ARSA (+23647); chr22:51110846-51110950/SHANK3 (−2172); chr22:51112010-51112026/SHANK3
(−1052); chr3:239126-239158/CHL1 (+863); chr3:2139976-2140037/CNTN4 (−543); chr3:2140148-2140181/CNT
N4 (−385); chr3:2141450-2141539/CNTN4 (+945); chr3:3221531-3221564/CRBN (−147); chr3:4909827-490990
6/BHLHE40 (−111230), ITPR1 (+374835); chr3:5228847-5228884/EDEM1 (−493); chr3:6902862-6902929/GRM
7 (+94); chr3:6903099-6903188/GRM7 (+342); chr3:6904488-6904532/GRM7 (+1708); chr3:8810076-8810151/
OXTR (+1186), CAV3 (+34628); chr3:9177902-9177995/RAD18 (−172790), SRGAP3 (+113362); chr3:9178130-
9178296/RAD18 (−173054), SRGAP3 (+113098); chr3:9550617-9550664/LHFPL4 (+44845), THUMPD3 (+1459
24); chr3:9595559-9595598/LHFPL4 (−93); chr3:9774197-9774246/BRPF1 (+788); chr3:9791997-9792057/OG
G1 (+399); chr3:9885473-9885516/RPUSD3 (+207); chr3:9993933-9993964/PRRT3 (+129); chr3:10028415-10
028473/TMEM111 (+78); chr3:10068005-10068058/FANCD2 (−81); chr3:10183396-10183460/VHL (+109); chr3:
10184561-10184583/IRAK2 (−21991), VHL (+1253); chr3:10857718-10857735/SLC6A11 (−190); chr3:1103535
3-11035382/SLC6A1 (+948); chr3:11079151-11079297/HRH1 (−116990), SLC6A1 (+44804); chr3:11762053-11
762076/VGLL4 (−76667), TAMM41 (+126287); chr3:12045920-12045978/SYN2 (+87); chr3:12046353-1204637
7/SYN2 (+503); chr3:12330230-12330283/TIMP4 (−129406), PPARG (−62744); chr3:12525721-12525801/TSEN
2 (−249); chr3:12705173-12705265/RAF1 (+481); chr3:12838451-12838507/CAND2 (+308); chr3:12910477-129
10504/RPL32 (−28542), IQSEC1 (+204126); chr3:13008598-13008692/RPL32 (−126696), IQSEC1 (+105972); c
hr3:13009453-13009475/RPL32 (−127515), IQSEC1 (+105153); chr3:13035667-13035681/RPL32 (−153725), IQ
SEC1 (+78943); chr3:13107888-13107921/RPL32 (−225956), IQSEC1 (+6712); chr3:13115122-13115176/IQSE
C1 (−532); chr3:13246005-13246037/IQSEC1 (−131404), NUP210 (+215788); chr3:13324029-13324073/IQSEC
1 (−209434), NUP210 (+137758); chr3:13456169-13456342/IQSEC1 (−341639), NUP210 (+5553); chr3:1359041
4-13590447/FBLN2 (−194); chr3:13590895-13590909/FBLN2 (+277); chr3:13696546-13696627/FBLN2 (+10596
2), WNT7A (+225031); chr3:14597400-14597427/C3orf20 (−119192), GRIP2 (−13826); chr3:14644713-1464474
9/C3orf20 (−71875), GRIP2 (−61143); chr3:14852205-14852212/FGD5 (−8260), C3orf20 (+135603); chr3:149898
21-14989845/NR2C2 (+597); chr3:15373663-15373720/SH3BP5 (+412); chr3:15901213-15901301/ANKRD28
(−204); chr3:16306251-16306326/OXNAD1 (−378), DPH3 (+207); chr3:16925532-16925583/DAZL (−278552), PL
CL2 (−49024); chr3:16926220-16926261/DAZL (−279235), PLCL2 (−48341); chr3:17784397-17784451/TBC1D5
(−184); chr3:19988994-19989023/RAB5A (+437); chr3:22413911-22413959/UBE2E2 (−830849), ZNF385D (−621
119); chr3:22414085-22414308/UBE2E2 (−830587), ZNF385D (−621381); chr3:23244247-23244293/UBE2E2
(−514); chr3:23848756-23848806/UBE2E1 (+1397), NKIRAS1 (+109756); chr3:24535849-24535911/THRB (+433);
chr3:24871177-24871245/RARB (−598623), THRB (−334898); chr3:27410843-27410882/NEK10 (+49); chr3:2
7764472-27764595/EOMES (−749); chr3:27765362-27765400/EOMES (−1596); chr3:27772107-27772184/CMC
1 (−510978), EOMES (−8361); chr3:32279432-32279558/CMTM8 (−676); chr3:32443051-32443152/CMTM7 (+9
939), CMTM6 (+101301); chr3:32501530-32501625/CMTM6 (+42825), CMTM7 (+68415); chr3:32543625-3254
3690/CMTM6 (+745); chr3:32822276-32822312/TRIM71 (−37216), CNOT10 (+95596); chr3:32859027-3285925
5/TRIM71 (−369); chr3:32860306-32860466/TRIM71 (+876); chr3:33156052-33156075/CRTAP (+614); chr3:334
81629-33481683/UBP1 (+241); chr3:33481864-33481890/UBP1 (+20); chr3:35680735-35680814/ARPP21 (−30
74); chr3:36422058-36422076/STAC (−30); chr3:36805537-36805575/DCLK3 (−24204), TRANK1 (+180992); chr
3:36806130-36806178/DCLK3 (−24802), TRANK1 (+180394); chr3:38035701-38035773/VILL (+659); chr3:3806
5892-38065948/PLCD1 (+358); chr3:38070670-38070745/PLCD1 (−4430); chr3:38081009-38081153/DLEC1
(+385); chr3:38081286-38081338/DLEC1 (+616); chr3:38179884-38179984/ACAA1 (−1201), MYD88 (−35); chr3:3
8206874-38206942/OXSR1 (−118); chr3:38495175-38495213/ACVR2B (−596); chr3:38691213-38691468/SCN5
A (−4074); chr3:39222468-39222493/CSRNP1 (−27379), XIRP1 (+11604); chr3:40494421-40494471/RPL14 (−43
55); chr3:40566536-40566563/ZNF621 (+174); chr3:40657189-40657325/CTNNB1 (−583685), ZNF621 (+90881);
chr3:41962687-41962817/ULK4 (+40908), CTNNB1 (+721810); chr3:42632455-42632475/NKTR (−9682), SE
C22C (−8945); chr3:42947809-42947832/ZNF662 (+163); chr3:43663169-43663221/ANO10 (+365); chr3:44040
026-44040056/C3orf23 (−339903), ABHD5 (+307666); chr3:44622682-44622730/ZNF660 (−3750); chr3:457305
63-45730623/SACM1L (−161); chr3:46506189-46506216/LTF (+395); chr3:46940054-46940076/NBEAL2 (−8110
8), PTH1R (+16326); chr3:47021024-47021079/NBEAL2 (−121); chr3:47050892-47051141/NBEAL2 (+29844),
SETD2 (+154450); chr3:47323677-47323729/KIF9 (+634); chr3:47620018-47620058/CSPG5 (+321); chr3:4782
3549-47823588/SMARCC1 (−164); chr3:47830232-47830269/DHX30 (−14148), SMARCC1 (−6846); chr3:483426
91-48342763/NME6 (+121); chr3:48520644-48520747/SHISA5 (−5755), SHISA5 (+20965); chr3:48646851-4864
6941/UQCRC1 (+202); chr3:48694155-48694226/TMEM89 (−35002), CELSR3 (+15790); chr3:48699089-48699
120/TMEM89 (−39916), CELSR3 (+10876); chr3:48723487-48723576/NCKIPSD (−198); chr3:48884753-488847
94/PRKAR2A (+496); chr3:48936247-48936303/SLC25A20 (+151); chr3:49203723-49203742/LAMB2 (−33134),
KLHDC8B (−5285); chr3:49394996-49395066/GPX1 (+760); chr3:49448641-49448690/TCTA (−973), RHOA (+8
60); chr3:49449439-49449454/TCTA (−192), RHOA (+79); chr3:49726560-49726623/RNF123 (−398), MST1 (−39
6); chr3:49727197-49727230/MST1 (−1018), RNF123 (+224); chr3:49967131-49967160/MON1A (+299); chr3:5
0203599-50203666/GNAT1 (−25410), SEMA3F (+10785); chr3:50312233-50312384/C3orf45 (−4209); chr3:5031
2997-50313020/C3orf45 (−3509); chr3:50313541-50313608/C3orf45 (−2943); chr3:50313795-50313931/C3orf45
(−2655); chr3:50314074-50314100/C3orf45 (−2431); chr3:50337069-50337166/HYAL3 (−798); chr3:50358957-5
0358980/HYAL1 (−17965), HYAL2 (+1312); chr3:50378696-50378733/RASSF1 (−348); chr3:50382960-5038301
8/RASSF1 (−4622), ZMYND10 (+167); chr3:50383244-50383263/RASSF1 (−4887), ZMYND10 (−98); chr3:51422
243-51422315/MANF (−413); chr3:51976266-51976338/RRP9 (−380), PARP3 (−59); chr3:52016827-52016853/A
CY1 (−460); chr3:52090184-52090241/DUSP7 (+248); chr3:52232402-52232453/ALAS1 (+329); chr3:52279836-
52279864/PPM1M (+41); chr3:52443863-52443906/PHF7 (−642), BAP1 (+124); chr3:52529184-52529415/STA
B1 (−56); chr3:53195173-53195238/PRKCD (−17); chr3:53915846-53915872/ACTR8 (+370); chr3:54155099-54
155169/CACNA2D3 (−1559); chr3:54155493-54155524/CACNA2D3 (−1184); chr3:54156629-54156654/CACNA
2D3 (−51); chr3:55517291-55517334/LRTM1 (−555241), WNT5A (+4018); chr3:55523927-55524031/WNT5A (−2
648); chr3:56502766-56502800/ERC2 (−392); chr3:56716417-56716515/ERC2 (−214075), ARHGEF3 (+396870);
chr3:56950302-56950373/ERC2 (−447947), ARHGEF3 (+162998); chr3:57678584-57678760/ARF4 (−95457),
SLMAP (−64502); chr3:57994158-57994203/FLNB (+54); chr3:57994507-57994542/FLNB (+398); chr3:5857263
4-58572799/FAM107A (−9226), FAM3D (+79844); chr3:61236910-61236965/FHIT (+195); chr3:61549360-6154
9442/PTPRG (+2158), FEZF2 (+809789); chr3:62304523-62304566/FEZF2 (+54645), PTPRG (+757302); chr3:
62354385-62354445/FEZF2 (+4775), PTPRG (+807172); chr3:62354793-62354899/FEZF2 (+4344), PTPRG
(+807603); chr3:62356220-62356366/FEZF2 (+2897), PTPRG (+809050); chr3:62356645-62356792/FEZF2 (+24
71), PTPRG (+809476); chr3:62357193-62357278/FEZF2 (+1954), PTPRG (+809993); chr3:62357671-623577
35/FEZF2 (+1487), PTPRG (+810460); chr3:62358117-62358336/FEZF2 (+963); chr3:62362737-62362816/FE
ZF2 (−3587); chr3:62362917-62363098/FEZF2 (−3818); chr3:62364177-62364279/FEZF2 (−5038), CADPS (+49
6836); chr3:63263997-63264062/SYNPR (+116); chr3:64253677-64253704/PRICKLE2 (−42560), ADAMTS9 (+4
19674); chr3:65342388-65342522/ADAMTS9 (−669090), MAGI1 (+682054); chr3:65535103-65535138/ADAMT
S9 (−861756), MAGI1 (+489388); chr3:65535320-65535366/ADAMTS9 (−861978), MAGI1 (+489166); chr3:6558
2645-65582716/ADAMTS9 (−909316), MAGI1 (+441828); chr3:65582962-65583074/ADAMTS9 (−909653), MAG
I1 (+441491); chr3:66848772-66848847/LRIG1 (−297965), SUCLG2 (+856228); chr3:67706430-67706499/SUC
LG2 (−1427); chr3:68056775-68056790/FAM19A1 (+16049), FAM19A4 (+924978); chr3:68664546-68664589/F
AM19A4 (+317193), FAM19A1 (+623834); chr3:68981815-68981851/FAM19A4 (−72); chr3:69591127-69591310/
MITF (−197367), FRMD4B (−155789); chr3:69591415-69591779/MITF (−196989), FRMD4B (−156167); chr3:695
91978-69592068/MITF (−196563), FRMD4B (−156593); chr3:71276002-71276112/FOXP1 (+18259); chr3:71803
784-71803826/GPR27 (+604); chr3:71804135-71804161/GPR27 (+947); chr3:72397134-72397248/PROK2 (−56
2834), RYBP (+98583); chr3:72704342-72704381/RYBP (−208588), SHQ1 (+193236); chr3:73673708-7367381
2/PDZRN3 (+312); chr3:74664021-74664045/CNTN3 (−93690); chr3:75451895-75451969/CNTN3 (−881589), F
RG2C (−261549); chr3:75485084-75485152/CNTN3 (−914775), FRG2C (−228363); chr3:75667874-75667944/F
RG2C (−45572); chr3:75719716-75719779/FRG2C (+6267), ZNF717 (+114507); chr3:75955811-75955872/ZNF
717 (−121587); chr3:75955997-75956026/ZNF717 (−121757); chr3:85008726-85008802/CADM2 (+631); chr3:85
010802-85010876/CADM2 (+2706); chr3:87138256-87138509/CHMP2B (−138030), VGLL3 (−98126); chr3:8810
7828-88107877/CGGBP1 (+293); chr3:88198750-88198771/C3orf38 (−132); chr3:97483768-97483833/ARL6
(+206); chr3:97541367-97541433/AK296966 (+516); chr3:99535942-99536014/C3orf26 (−700); chr3:100119377-1
00119448/TOMM70A (+829); chr3:101498453-101498468/FAM55C (+432); chr3:105072767-105072783/ALCA
M (−12782); chr3:105088031-105088087/ALCAM (+2502), CBLB (+499828); chr3:105586963-105587027/CBLB
(+892); chr3:107150969-107151035/BBX (−90781); chr3:109055574-109055591/DPPA4 (+836); chr3:1090559
34-109056037/DPPA4 (+433); chr3:111579531-111579579/PHLDB2 (+987); chr3:112052141-112052251/CD20
0 (+280); chr3:112930437-112930455/BOC (−929); chr3:112930614-112930655/BOC (−740); chr3:115510869-1
15510949/GAP43 (+168758), LSAMP (+653476); chr3:117716124-117716213/NONE; chr3:119041954-119041
987/ARHGAP31 (+28751), TMEM39A (+140500); chr3:119396003-119396052/COX17 (+215); chr3:120004036-
120004079/GPR156 (−40733), FSTL1 (+165860); chr3:120025699-1 20025740/GPR156 (−62395), FSTL1 (+144
198); chr3:120169977-120170066/FSTL1 (−104); chr3:120170241-120170266/FSTL1 (−336); chr3:120461634-1
20461714/RABL3 (−290), GTF2E1 (+116); chr3:121553253-121553299/EAF2 (−758), IQCB1 (+650); chr3:12155
3765-121553827/EAF2 (−238), IQCB1 (+130); chr3:121902950-121902990/CASR (+440); chr3:121903219-121
903323/CASR (+741); chr3:122103018-122103066/CCDC58 (−968), FAM162A (+19); chr3:122296781-1222968
23/PARP15 (+353); chr3:122640753-122640946/SEMA5B (+105726), DIRC2 (+126949); chr3:122641209-1226
41233/SEMA5B (+105355), DIRC2 (+127320); chr3:122745882-122745939/SEMA5B (+665); chr3:122920878-1
22920922/SEC22A (+126); chr3:123167006-123167028/ADCY5 (+375); chr3:123168568-123168581/ADCY5
(−1183); chr3:123603185-123603206/MYLK (−47); chr3:124860569-124860811/HEG1 (−85888), SLC12A8 (+7091
9); chr3:124931263-124931305/SLC12A8 (+325); chr3:125076101-125076148/SLC12A8 (−144516), ZNF148
(+18073); chr3:125076284-125076346/SLC12A8 (−144706), ZNF148 (+17883); chr3:125094160-125094185/ZNF
148 (+25); chr3:125676967-125677121/ALG1L (−21157), ROPN1B (−10984); chr3:125690940-125691029/ROP
N1B (+2957), SLC41A3 (+112149); chr3:125802806-125802854/SLC41A3 (+304); chr3:126108190-126108251/
KLF15 (−31985), ZXDC (+86541); chr3:126113657-126113794/KLF15 (−37490), ZXDC (+81036); chr3:1267016
27-126701698/TXNRD3 (−327696), PLXNA1 (−5774); chr3:126854505-126854536/PLXNA1 (+147084), TPRA1
(+455047); chr3:127143768-127143929/TPRA1 (+165719), PLXNA1 (+436412); chr3:127392263-127392321/A
BTB1 (+511); chr3:127480529-127480590/MGLL (+61491), ABTB1 (+88779); chr3:127633950-127634094/SEC
61A1 (−137190), MGLL (−91971); chr3:127795163-127795229/SEC61A1 (+23984), RUVBL1 (+47475); chr3:127
795754-127796073/SEC61A1 (+24702), RUVBL1 (+46757); chr3:128202293-128202349/DNAJB8 (−16230), GA
TA2 (+9709); chr3:128209346-128209487/DNAJB8 (−23326), GATA2 (+2613); chr3:128210668-128210712/DN
AJB8 (−24599), GATA2 (+1340); chr3:128210983-128211669/GATA2 (+704); chr3:128212054-128212272/GAT
A2 (−133); chr3:128216646-128216698/GATA2 (−4642); chr3:128274069-128274230/GATA2 (−62120), RPN1
(+95569); chr3:128274655-128274701/GATA2 (−62648), RPN1 (+95041); chr3:128307103-128307158/GATA2 (−9
5101), RPN1 (+62588); chr3:128399491-128399546/RAB7A (−45460), RPN1 (−29800); chr3:128445228-128445
322/RAB7A (+296); chr3:128564814-128564891/ACAD9 (−33480), RAB7A (+119874); chr3:128598634-128598
701/ACAD9 (+335); chr3:128720143-128720163/GP9 (−59492), ACAD9 (+121820); chr3:128720347-12872041
8/GP9 (−59262), ACAD9 (+122050); chr3:128720534-128720566/GP9 (−59095), ACAD9 (+122217); chr3:12872
2697-128722739/GP9 (−56927), ACAD9 (+124385); chr3:128746028-128746092/GP9 (−33585), ACAD9 (+1477
27); chr3:128764717-128764778/GP9 (−14897), ACAD9 (+166415); chr3:128765030-128765049/GP9 (−14605),
ACAD9 (+166707); chr3:128765157-128765230/GP9 (−14451), ACAD9 (+166861); chr3:128840286-128840346/
RAB43 (+331); chr3:128902246-128902299/CNBP (+537); chr3:129034100-129034145/H1FX (+997); chr3:129
297146-129297230/PLXND1 (+28394), H1FOO (+35131); chr3:129312876-129312984/PLXND1 (+12652), H1F
OO (+50873); chr3:129345804-1 29345876/PLXND1 (−20258), TMCC1 (+253562); chr3:129693200-129693304/
TRH (+138); chr3:129693573-129693612/TRH (+479); chr3:129693841-129693950/TRH (+782); chr3:1296943
47-129694766/ALG1L2 (−106117), TRH (+1443); chr3:129829971-129829979/COL6A5 (−234384), ALG1L2 (+2
9301); chr3:129830161-129830212/COL6A5 (−234172), ALG1L2 (+29513); chr3:130064610-130064780/COL6A
5 (+336); chr3:130236064-130236173/COL6A6 (−43059), COL6A5 (+171760); chr3:130745247-130745318/NE
K11 (−411), ASTE1 (+363); chr3:131245950-131246019/ACPP (−790226), MRPL3 (−24125); chr3:132757480-13
2757536/TMEM108 (+337); chr3:133393277-133393462/TF (−71430), TOPBP1 (−12633); chr3:133393594-1333
93621/TF (−71192), TOPBP1 (−12871); chr3:133465292-133465369/TF (+531); chr3:133613913-133613942/RA
B6B (+763); chr3:133748366-133748401/SLCO2A1 (+536); chr3:134093552-134093626/AMOTL2 (−183); chr3:
134369519-134369552/KY (+328); chr3:134370128-134370181/KY (−291); chr3:136470826-136470865/STAG1
(+399); chr3:137481873-137481935/SOX14 (−1230); chr3:137483438-137483513/SOX14 (+342); chr3:137484
026-137484084/SOX14 (+921); chr3:137487788-137487850/CLDN18 (−229839), SOX14 (+4685); chr3:137488
004-137488090/CLDN18 (−229611), SOX14 (+4913); chr3:137489699-137489875/CLDN18 (−227871), SOX14
(+6653); chr3:137833848-137833932/DZIP1L (+561); chr3:138067979-138068012/MRAS (+488); chr3:1381542
77-138154497/ESYT3 (+972); chr3:138657495-138657617/PIK3CB (−179371), FOXL2 (+8426); chr3:13865834
4-138658414/PIK3CB (−180194), FOXL2 (+7603); chr3:138658579-138658703/PIK3CB (−180456), FOXL2 (+73
41); chr3:138658864-138659080/PIK3CB (−180787), FOXL2 (+7010); chr3:138662180-138662281/PIK3CB (−18
4046), FOXL2 (+3751); chr3:138663261-138663390/PIK3CB (−185141), FOXL2 (+2656); chr3:138663728-1386
64072/PIK3CB (−185715), FOXL2 (+2082); chr3:138664416-138664457/PIK3CB (−186252), FOXL2 (+1545); chr
3:138664769-138664802/PIK3CB (−186601), FOXL2 (+1196); chr3:138665336-138665396/FOXL2 (+616); chr3:
138665528-138665539/FOXL2 (+448); chr3:138668685-138668757/FOXL2 (−2739); chr3:138669109-13866914
0/FOXL2 (−3143); chr3:139258077-139258159/RBP1 (+553); chr3:140770302-140770407/SPSB4 (−388); chr3:1
40770590-140770643/SPSB4 (−126); chr3:140951182-140951233/ACPL2 (+526); chr3:141030471-141030521/
ZBTB38 (−12559), ACPL2 (+79814); chr3:141867786-141867861/TFDP2 (+562); chr3:141868521-141868591/T
FDP2 (−170); chr3:141944252-141944291/GK5 (+177); chr3:142681995-142682049/PAQR9 (+156); chr3:14283
8319-142838479/CHST2 (−219); chr3:142838647-142838876/CHST2 (+144); chr3:142839073-142839204/CHS
T2 (+521); chr3:142839526-142839562/CHST2 (+926); chr3:142839689-142839783/CHST2 (+1118), SLC9A9
(+727637); chr3:142839902-142839944/CHST2 (+1305), SLC9A9 (+727450); chr3:142840128-142840221/CHS
T2 (+1557), SLC9A9 (+727198); chr3:143048656-143048692/CHST2 (+210056), SLC9A9 (+518699); chr3:143
566706-143566766/SLC9A9 (+637); chr3:147106175-147106207/PLSCR1 (−843563), ZIC4 (+15880); chr3:147
108531-147108586/PLSCR1 (−845931), ZIC4 (+13512); chr3:147109766-147110010/PLSCR1 (−847260), ZIC4
(+12183); chr3:147110115-147110228/PLSCR1 (−847544), ZIC4 (+11899); chr3:147111378-147111398/PLSCR
1 (−848760), ZIC4 (+10683); chr3:147111560-147111693/PLSCR1 (−848999), ZIC4 (+10444); chr3:147113725-
147113789/PLSCR1 (−851129), ZIC4 (+8314); chr3:147114105-147114135/PLSCR1 (−851492), ZIC4 (+7951); c
hr3:147114322-147114368/PLSCR1 (−851717), ZIC4 (+7726); chr3:147115069-147115106/PLSCR1 (−852460),
ZIC4 (+6983); chr3:147122686-147122718/ZIC1 (−4479), ZIC4 (−631); chr3:147125113-147125260/ZIC4 (−3116),
ZIC1 (−1994); chr3:147127565-147127675/ZIC1 (+439); chr3:147127913-147128243/ZIC1 (+897); chr3:14712
8422-147128669/ZIC1 (+1365); chr3:147129732-147129799/ZIC1 (+2585); chr3:147131177-147131220/ZIC1
(+4018); chr3:147137001-147137075/ZIC1 (+9857); chr3:147137371-147137428/ZIC1 (+10219); chr3:14713899
9-147139250/ZIC1 (+11944); chr3:147141261-147141322/ZIC1 (+14111); chr3:147142152-147142382/ZIC1
(+15086); chr3:148415840-148415861/AGTR1 (+193); chr3:150481320-150481343/SIAH2 (−69); chr3:150803000-
150803025/MED12L (−1572); chr3:150804896-150804936/MED12L (+331); chr3:152879802-152879855/RAP2
B (−200); chr3:153839953-153840317/ARHGEF26 (+1343), DHX36 (+202151); chr3:154145565-154145627/DH
X36 (−103310), GPR149 (+1908); chr3:154146395-154146488/DHX36 (−104156), GPR149 (+1062); chr3:15479
7778-154797835/MME (−146); chr3:155588123-155588154/GMPS (−186); chr3:156009300-156009318/KCNAB
1 (+170972), SSR3 (+263664); chr3:156272710-156272763/SSR3 (+236); chr3:156392240-156392266/TIPARP
(−2200); chr3:156534098-156534392/TIPARP (+139792), CCNL1 (+344237); chr3:156892934-156893032/PTX
3 (−261597), CCNL1 (−14501); chr3:157812258-157812436/VEPH1 (−591211), SHOX2 (+11605); chr3:1578136
05-157813669/VEPH1 (−592501), SHOX2 (+10315); chr3:157820869-157821368/VEPH1 (−599983), SHOX2
(+2833); chr3:157822880-157823011/RSRC1 (−4946), SHOX2 (+1006); chr3:157823120-157823138/RSRC1 (−47
63), SHOX2 (+823); chr3:157824147-157824211/RSRC1 (−3713), SHOX2 (−227); chr3:157824909-157825059/
RSRC1 (−2908), SHOX2 (−1032); chr3:158288819-158288886/MLF1 (−100); chr3:158449909-158449969/RARR
ES1 (+336); chr3:160116675-160116718/SMC4 (−733), IFT80 (+623); chr3:160116985-160117031/SMC4 (−422),
IFT80 (+312); chr3:160167989-160168070/TRIM59 (−404); chr3:160283107-160283131/KPNA4 (+257); chr3:1
67813746-167813780/GOLIM4 (−346); chr3:169382162-169382201/MECOM (−619); chr3:169383988-16938403
3/MECOM (−2448); chr3:169899327-169899548/PHC3 (+99); chr3:170075865-170075923/SKIL (−1517); chr3:1
70137345-170137884/CLDN11 (+962); chr3:170303143-170303379/SLC7A14 (+602); chr3:170303495-170303
554/SLC7A14 (+338); chr3:171175873-171175956/SLC2A2 (−431147), TNIK (+2282); chr3:171528549-171528
609/PLD1 (−75); chr3:172165785-172165866/GHSR (+420); chr3:172166513-172166672/GHSR (−347); chr3:17
2166894-172166999/GHSR (−701); chr3:172167474-172167595/GHSR (−1289); chr3:173302669-173302734/N
LGN1 (+186458); chr3:175655182-175655239/NONE; chr3:178865811-178865876/PIK3CA (−467); chr3:17897
9132-178979157/KCNMB3 (+5693), PIK3CA (+112834); chr3:179065937-179065973/MFN1 (+475); chr3:17932
2564-179322607/MRPL47 (−152), NDUFB5 (+11); chr3:179754193-179754238/PEX5L (+301); chr3:179754760-
179754803/PEX5L (−265); chr3:179754989-179755072/PEX5L (−514); chr3:180320055-180320087/TTC14 (+15
3); chr3:181413069-181413278/DNAJC19 (−705612), SOX2 (−16538); chr3:181430367-181430443/SOX2 (+693);
chr3:181444525-181444612/SOX2 (+14857); chr3:181444949-181444988/SOX2 (+15257); chr3:181445725-
181445799/SOX2 (+16050); chr3:182698560-182698584/DCUN1D1 (−246); chr3:182897108-182897157/B3GN
T5 (−73899), LAMP3 (−16466); chr3:183735352-183735380/ABCC5 (+361); chr3:183735543-183735597/ABCC
5 (+157); chr3:184017109-184017124/PSMD2 (+95); chr3:184054379-184054446/FAM131A (−863); chr3:18409
9205-184099331/THPO (−3336), CHRD (+1407); chr3:184286546-184286582/VPS8 (−243367), EPHB3 (+6977);
chr3:184301801-184301859/VPS8 (−228101), EPHB3 (+22243); chr3:184319400-184319469/VPS8 (−210496),
EPHB3 (+39848); chr3:184319843-184319873/VPS8 (−210073), EPHB3 (+40271); chr3:184321874-184322209/
VPS8 (−207889), EPHB3 (+42455); chr3:184405562-184405614/VPS8 (−124343), EPHB3 (+126001); chr3:1844
96482-184496611/VPS8 (−33384), EPHB3 (+216960); chr3:185654572-185654633/IGF2BP2 (−111776), TRA2B
(+1321); chr3:185654988-185655065/TRA2B (+897); chr3:185911588-185911606/ETV5 (−84696), DGKG (+16
8426); chr3:185911722-185911771/ETV5 (−84846), DGKG (+168276); chr3:186079771-186079818/DGKG (+22
8); chr3:186285068-186285079/DNAJB11 (−2878), TBCCD1 (+67); chr3:186490652-186490719/EIF4A2 (−1067
5), KNG1 (+55588); chr3:186542782-186542843/RFC4 (−18523), ADIPOQ (−17650); chr3:186543137-18654324
3/RFC4 (−18900), ADIPOQ (−17273); chr3:186543526-186543567/RFC4 (−19257), ADIPOQ (−16916); chr3:1873
87921-187388006/SST (+237); chr3:187676563-187676643/LPP (−254118), BCL6 (−213090); chr3:192125829-
192126115/FGF12 (+866); chr3:192126711-192126786/FGF12 (+89); chr3:192127373-192127556/FGF12 (−62
7); chr3:192127731-192127936/FGF12 (−996); chr3:192232819-192232849/HRASLS (−726084), FGF12 (−1059
96); chr3:192232952-192233003/HRASLS (−725940), FGF12 (−106140); chr3:192445387-192445421/HRASLS
(−513514), FGF12 (−318566); chr3:193587593-193587652/HES1 (−266308), OPA1 (+276690); chr3:193858882-
193858949/HES1 (+4985), CPN2 (+213141); chr3:193924363-193924403/HES1 (+70452), CPN2 (+147674); c
hr3:193987655-193987699/CPN2 (+84380), HES1 (+133746); chr3:194117484-194117769/LRRC15 (−27155),
GP5 (+2368); chr3:194118180-194118267/LRRC15 (−27752), GP5 (+1771); chr3:194118746-194118917/LRRC
15 (−28360), GP5 (+1163); chr3:194207367-194207382/ATP13A3 (−18407), TMEM44 (+146775); chr3:1942082
58-194208467/ATP13A3 (−19395), TMEM44 (+145787); chr3:194208855-194208926/ATP13A3 (−19923), TME
M44 (+145259); chr3:194406553-194406589/LSG1 (−13365), XXYLT1 (+585324); chr3:194407989-194407996/
LSG1 (−14787), XXYLT1 (+583902); chr3:194408769-194408838/LSG1 (−15598), XXYLT1 (+583091); chr3:194
705949-194706050/LSG1 (−312794), XXYLT1 (+285895); chr3:195270087-195270134/PPP1R2 (+113); chr3:19
5488911-195489066/MUC20 (+41236), MUC4 (+49855); chr3:195583592-195583665/MUC4 (−44785), TNK2
(+38803); chr3:195621979-195622064/TNK2 (+410); chr3:195634142-195634194/TNK2 (−11736), SDHAP1 (+829
82); chr3:195635757-195635785/TNK2 (−13339), SDHAP1 (+81379); chr3:195636295-195636337/TNK2 (−1388
4), SDHAP1 (+80834); chr3:195753067-195753108/SDHAP1 (−35938), TFRC (+55944); chr3:195808391-19580
8435/TFRC (+619); chr3:195886887-195887032/TFRC (−77928), ZDHHC19 (+51340); chr3:195947391-195947
506/OSTalpha (+4066), PCYT1A (+67135); chr3:196014427-196014503/PCYT1A (+119); chr3:196159284-196
159339/UBXN7 (+33); chr3:196255745-196255875/FBXO45 (−39915), C3orf43 (−13573); chr3:196359639-1963
59787/LRRC33 (−6943), FBXO45 (+63988); chr3:196367506-196367631/LRRC33 (+913); chr3:196730268-196
730371/PIGZ (−34616), MFI2 (+26367); chr3:197281858-197281920/BDH1 (+969); chr3:197282619-197282805/
BDH1 (+146); chr3:197463790-197463818/KIAA0226 (−31); chr3:197808349-197808380/LMLN (+121294); chr
3:197840548-197840692/LMLN (+153549); chr4:107281-107296/ZNF718 (−17113), ZNF718 (+54062); chr4:107
509-107875/ZNF718 (−16710), ZNF718 (+54465); chr4:206593-206607/ZNF718 (+82198), ZNF732 (+83344); c
hr4:206742-206813/ZNF718 (+82376), ZNF732 (+83166); chr4:298782-298821/ZNF141 (−32794), ZNF732 (−88
58); chr4:299021-299032/ZNF141 (−32569), ZNF732 (−9083); chr4:568653-568906/ZNF721 (−75338), PDE6B
(−50583); chr4:569116-569274/ZNF721 (−75753), PDE6B (−50168); chr4:569436-569460/ZNF721 (−76006), PDE6
B (−49915); chr4:569733-569933/ZNF721 (−76391), PDE6B (−49530); chr4:570257-570298/ZNF721 (−76836), P
DE6B (−49085); chr4:668655-668707/MYL5 (−3030), ATP5I (−554); chr4:682570-683014/MFSD7 (+181); chr4:77
9086-779189/CPLX1 (+40807), PCGF3 (+79565); chr4:779398-779461/CPLX1 (+40515), PCGF3 (+79857); chr
4:780406-780551/CPLX1 (+39466), PCGF3 (+80906); chr4:789693-789734/CPLX1 (+30231), PCGF3 (+90141);
chr4:820653-820702/CPLX1 (−733); chr4:967385-967415/DGKQ (−56); chr4:967621-967632/DGKQ (−283); chr
4:995539-995875/FGFRL1 (−10053), SLC26A1 (−8483); chr4:995994-996100/FGFRL1 (−9713), SLC26A1 (−882
3); chr4:996298-996495/FGFRL1 (−9363), SLC26A1 (−9173); chr4:996797-996882/SLC26A1 (−9616), FGFRL1
(−8920); chr4:1006166-1006198/FGFRL1 (+422); chr4:1049790-1049908/FGFRL1 (+44089), RNF212 (+57503);
chr4:1107280-1107324/RNF212 (+50); chr4:1164854-1164984/RNF212 (−57567), SPON2 (+1738); chr4:11652
87-1165449/RNF212 (−58016), SPON2 (+1289); chr4:1195837-1196010/SPON2 (−29267), CTBP1 (+46984); ch
r4:1210625-1210918/SPON2 (−44115), CTBP1 (+32136); chr4:1243309-1243334/CTBP1 (−414); chr4:1288919-
1288994/CRIPAK (−96383), MAEA (+5285); chr4:1303356-1303427/CRIPAK (−81948), MAEA (+19720); chr4:13
40250-1340280/CRIPAK (−45075), MAEA (+56593); chr4:1340688-1340718/CRIPAK (−44637), MAEA (+57031);
chr4:1395728-1395811/CRIPAK (+10430), FAM53A (+290218); chr4:1396697-1396779/CRIPAK (+11398), FA
M53A (+289250); chr4:1397026-1397259/CRIPAK (+11803), FAM53A (+288845); chr4:1398247-1398263/CRIP
AK (+12915), FAM53A (+287733); chr4:1398840-1398858/CRIPAK (+13509), FAM53A (+287139); chr4:139963
3-1399665/CRIPAK (+14309), FAM53A (+286339); chr4:1400511-1400671/CRIPAK (+15251), FAM53A (+2853
97); chr4:1400868-1400997/CRIPAK (+15593), FAM53A (+285055); chr4:1401522-1401642/CRIPAK (+16242),
FAM53A (+284406); chr4:1404066-1404120/CRIPAK (+18753), FAM53A (+281895); chr4:1408743-1408885/C
RIPAK (+23474), FAM53A (+277174); chr4:1408996-1409010/CRIPAK (+23663), FAM53A (+276985); chr4:151
4632-1514674/CRIPAK (+129313), FAM53A (+171335); chr4:1724485-1724592/TMEM129 (−1455), TACC3 (+1
322); chr4:1773167-1773254/FGFR3 (−21828), TACC3 (+49994); chr4:1773774-1773831/FGFR3 (−21236), TAC
C3 (+50586); chr4:1794382-1794459/FGFR3 (−618); chr4:1794956-1794972/FGFR3 (−75); chr4:1858688-18587
26/LETM1 (−733); chr4:1864233-1864268/WHSC1 (−8872), LETM1 (−6277); chr4:2042123-2042168/C4orf48 (−1
574); chr4:2042270-2042465/C4orf48 (−1352); chr4:2060155-2060173/NAT8L (−1075); chr4:2292778-2292805/
MXD4 (−29053), ZFYVE28 (+127578); chr4:2420996-2421068/ZFYVE28 (−662); chr4:2538114-2538151/RNF4
(+67338), TNIP2 (+219970); chr4:2765667-2765764/SH3BP2 (−54196), TNIP2 (−7613); chr4:2799751-2799817/T
NIP2 (−41681), SH3BP2 (−20128); chr4:2800771-2800828/TNIP2 (−42697), SH3BP2 (−19112); chr4:3294960-32
95041/RGS12 (−20873), HTT (+218593); chr4:3387376-3387498/HGFAC (−56289), RGS12 (+71563); chr4:3491
424-3491502/DOK7 (+26430), LRPAP1 (+42761); chr4:3533386-3533472/LRPAP1 (+795); chr4:3534128-3534
148/LRPAP1 (+86); chr4:3534547-3534604/LRPAP1 (−352); chr4:3768950-3768994/ADRA2C (+676); chr4:376
9224-3769559/ADRA2C (+1096), OTOP1 (+459229); chr4:3769665-3769695/ADRA2C (+1384), OTOP1 (+4589
41); chr4:3873965-3873974/ADRA2C (+105674), OTOP1 (+354651); chr4:3957127-3957154/ADRA2C (+18884
5), OTOP1 (+271480); chr4:4144584-4144633/OTOP1 (+84012), ADRA2C (+376313); chr4:4144850-4144882/
OTOP1 (+83755), ADRA2C (+376570); chr4:4145331-4145386/OTOP1 (+83262), ADRA2C (+377063); chr4:42
28168-4228188/OTOP1 (+443); chr4:4329357-4329454/D4S234E (−58178), ZBTB49 (+37482); chr4:4389172-4
389207/D4S234E (+1606), STX18 (+154585); chr4:4397982-4398058/D4S234E (+10436), STX18 (+145755); c
hr4:4765263-4765316/STX18 (−221515), MSX1 (−96102); chr4:4855192-4855230/STX18 (−311436), MSX1 (−61
81); chr4:4856557-4856593/MSX1 (−4817); chr4:4857362-4857407/MSX1 (−4007); chr4:4857560-4857620/MSX
1 (−3802); chr4:4858107-4858130/MSX1 (−3273); chr4:4859771-4859810/MSX1 (−1601); chr4:4863318-486339
3/MSX1 (+1964), CYTL1 (+157841); chr4:4866364-4866544/MSX1 (+5062), CYTL1 (+154743); chr4:4866799-4
866929/MSX1 (+5472), CYTL1 (+154333); chr4:4867453-4867482/MSX1 (+6076), CYTL1 (+153729); chr4:486
8384-4868464/MSX1 (+7032), CYTL1 (+152773); chr4:4868793-4868828/MSX1 (+7419), CYTL1 (+152386); ch
r4:4869000-4869131/MSX1 (+7674), CYTL1 (+152131); chr4:4869258-4869481/MSX1 (+7978), CYTL1 (+1518
27); chr4:4872032-4872059/MSX1 (+10654), CYTL1 (+149151); chr4:5710228-5710280/EVC (−2670), EVC2
(+40); chr4:5713232-5713326/EVC2 (−2985), EVC (+355); chr4:5868033-5868176/CRMP1 (+26680), EVC (+1551
81); chr4:5892082-5892109/CRMP1 (+2689), EVC (+179172); chr4:5892251-5892316/CRMP1 (+2501), EVC
(+179360); chr4:5894083-5894130/CRMP1 (+678); chr4:5894277-5894599/CRMP1 (+347); chr4:6201293-62013
49/JAKMIP1 (+997); chr4:6223904-6223950/WFS1 (−47650), JAKMIP1 (−21609); chr4:6381669-6381831/WFS1
(+110173), PPP2R2C (+175655); chr4:6392268-6392279/WFS1 (+120697), PPP2R2C (+165131); chr4:64804
91-6480532/PPP2R2C (+76893), WFS1 (+208935); chr4:6642677-6642696/MRFAP1 (+242); chr4:6665813-66
66008/S100P (−29655), MRFAP1 (+23466); chr4:6666118-6666219/S100P (−29397), MRFAP1 (+23724); chr4:6
783872-6783911/KIAA0232 (−567); chr4:6784167-6784224/KIAA0232 (−263); chr4:7033760-7033861/TADA2B
(−11345), TBC1D14 (+122316); chr4:7072880-7072953/GRPEL1 (−3117); chr4:7195266-7195308/SORCS2 (+91
3); chr4:7444569-7444627/PSAPL1 (−7898), AFAP1 (+497055); chr4:7941092-7941129/AFAP1 (+542); chr4:79
70346-7970431/AFAP1 (−28736), ABLIM2 (+190170); chr4:8073656-8073691/AFAP1 (−132021), ABLIM2 (+868
85); chr4:8116361-8116503/AFAP1 (−174779), ABLIM2 (+44127); chr4:8159491-8159548/AFAP1 (−217867), AB
LIM2 (+1039); chr4:8160355-8160429/ABLIM2 (+167); chr4:8200133-8200210/SH3TC1 (−888); chr4:8201488-8
201550/SH3TC1 (+459); chr4:8281163-8281203/HTRA3 (+9694), ACOX3 (+161269); chr4:8582324-8582394/G
PR78 (+142); chr4:8582619-8582656/GPR78 (+421); chr4:8651788-8651874/CPZ (+57444), HMX1 (+221712);
chr4:8859048-8859381/HMX1 (+14328), CPZ (+264828); chr4:8860146-8860180/HMX1 (+13380), CPZ (+2657
76); chr4:8861546-8861605/HMX1 (+11967), CPZ (+267189); chr4:8861753-8861918/HMX1 (+11707), CPZ (+2
67449); chr4:8863238-8863323/HMX1 (+10262), CPZ (+268894); chr4:8863536-8863568/HMX1 (+9991), CPZ
(+269165); chr4:8864683-8864763/HMX1 (+8820), CPZ (+270336); chr4:8866160-8866264/HMX1 (+7331), CPZ
(+271825); chr4:8868846-8868931/HMX1 (+4654), CPZ (+274502); chr4:8869271-8869368/HMX1 (+4223), CP
Z (+274933); chr4:8873114-8873140/HMX1 (+416); chr4:8874538-8874580/HMX1 (−1016); chr4:8874696-8874
772/HMX1 (−1191); chr4:8876036-8876229/HMX1 (−2590); chr4:8879594-8879647/LOC650293 (−71856), HMX1
(−6078); chr4:8893344-8893391/LOC650293 (−58109), HMX1 (−19825); chr4:8893726-8893815/LOC650293 (−5
7706), HMX1 (−20228); chr4:8895084-8895128/LOC650293 (−56371), HMX1 (−21563); chr4:8896098-8896117/L
OC650293 (−55369), HMX1 (−22565); chr4:8965298-8965357/USP17 (−251803), LOC650293 (+13851); chr4:90
02285-9002329/USP17 (−214824), LOC650293 (+50830); chr4:9002823-9003020/USP17 (−214209), LOC65029
3 (+51445); chr4:9003141-9003231/USP17 (−213945), LOC650293 (+51709); chr4:9154060-9154103/USP17
(−63049), LOC650293 (+202605); chr4:9382952-9382978/DEFB131 (−63295), LOC728405 (+13365); chr4:95345
95-9534650/DRD5 (−248635), DEFB131 (+88363); chr4:9783096-9783125/DRD5 (−147); chr4:9783424-978350
0/DRD5 (+204); chr4:9783620-9783681/DRD5 (+393); chr4:10463048-10463072/ZNF518B (−4028); chr4:10589
431-10589535/ZNF518B (−130451), CLNK (+96903); chr4:11370513-11370562/CLNK (−684152), HS3ST1 (+59
999); chr4:13524252-13524529/RAB28 (−38402), NKX3-2 (+21723); chr4:13524972-13525121/RAB28 (−39058),
NKX3-2 (+21067); chr4:13525428-13525473/RAB28 (−39462), NKX3-2 (+20663); chr4:13525604-13525825/RA
B28 (−39726), NKX3-2 (+20399); chr4:13525942-13526005/RAB28 (−39985), NKX3-2 (+20140); chr4:13526628-
13526768/RAB28 (−40709), NKX3-2 (+19416); chr4:13532587-13532654/RAB28 (−46632), NKX3-2 (+13493); c
hr4:13540903-13540945/RAB28 (−54935), NKX3-2 (+5190); chr4:13541348-13541749/RAB28 (−55560), NKX3-
2 (+4565); chr4:13543581-13543972/RAB28 (−57788), NKX3-2 (+2337); chr4:13544080-13544211/RAB28 (−581
57), NKX3-2 (+1968); chr4:13545386-13545593/NKX3-2 (+624); chr4:13548590-13548634/NKX3-2 (−2498); chr
4:13628913-13628956/BOD1L (+393); chr4:14864562-14864612/CPEB2 (−139711); chr4:15004994-15005113/
CPEB2 (+756); chr4:16084819-16085166/PROM1 (−7252), TAPT1 (+143168); chr4:16085302-16085351/PROM
1 (−7586), TAPT1 (+142834); chr4:16085602-16085674/PROM1 (−7897), TAPT1 (+142523); chr4:16228936-162
28990/TAPT1 (−802), BC010030 (+677); chr4:16229130-16229202/TAPT1 (−1005), BC010030 (+880); chr4:175
13309-17513378/CLRN2 (−3444), QDPR (+513); chr4:17578667-17578690/LAP3 (−248); chr4:17578863-17578
894/LAP3 (−48); chr4:17783481-17783609/DCAF16 (+28836), MED28 (+167272); chr4:18023666-18023721/LC
ORL (−211); chr4:20254774-20255026/SLIT2 (−335); chr4:20255414-20255511/SLIT2 (+228); chr4:20256286-2
0256382/PACRGL (−445702), SLIT2 (+1099); chr4:20256662-20256676/PACRGL (−445367), SLIT2 (+1434); ch
r4:21305873-21305986/KCNIP4 (+393388), PACRGL (+603894); chr4:21950566-21950721/KCNIP4 (−251326),
GPR125 (+567028); chr4:24585643-24585691/DHX15 (+517); chr4:24801325-24801867/SOD3 (+4511), LGI2
(+230818); chr4:24802053-24802319/SOD3 (+5101), LGI2 (+230228); chr4:26030566-26030635/RBPJ (−29182
8), C4orf52 (+114787); chr4:26275398-26275516/RBPJ (−46972), C4orf52 (+359643); chr4:26322308-2632234
3/RBPJ (−103); chr4:26323036-26323080/RBPJ (+629); chr4:26863173-26863190/STIM2 (+869); chr4:3072003
4-30720054/PCDH7 (−1993); chr4:30724063-30724126/PCDH7 (+2058); chr4:30724538-30724656/PCDH7 (+2
560); chr4:36246208-36246257/ARAP2 (−254); chr4:37245350-37245430/RELL1 (+442609), DTHD1 (+959746);
chr4:37246361-37246489/RELL1 (+441574), DTHD1 (+960781); chr4:37247238-37247293/RELL1 (+440733),
DTHD1 (+961622); chr4:37688502-37688517/RELL1 (−511); chr4:37893212-37893243/TBC1D1 (+523); chr4:3
9448646-39448926/RPL9 (+11421), KLB (+40313); chr4:39640050-39640114/C4orf34 (+399); chr4:39979371-3
9979387/PDS5A (+197); chr4:39979520-39979543/PDS5A (+44); chr4:40632470-40632528/RBM47 (−616); chr
4:40859061-40859189/NSUN7 (+107211), APBB2 (+357510); chr4:41258789-41259085/UCHL1 (+39); chr4:41
362763-41362780/LIMCH1 (−32); chr4:41747769-41747888/PHOX2B (+3158), LIMCH1 (+385025); chr4:41748
012-41748143/PHOX2B (+2909), LIMCH1 (+385274); chr4:41748767-41748856/PHOX2B (+2175), LIMCH1 (+3
86008); chr4:41753917-41754030/PHOX2B (−2987); chr4:41867516-41867612/PHOX2B (−116577), TMEM33
(−69573); chr4:41880420-41880554/PHOX2B (−129500), TMEM33 (−56650); chr4:41881118-41881201/PHOX2B
(−130173), TMEM33 (−55977); chr4:41882113-41882162/PHOX2B (−131151), TMEM33 (−54999); chr4:41882533-
41882651/PHOX2B (−131605), TMEM33 (−54545); chr4:42152766-42152961/SHISA3 (−246992), SLC30A9 (+1
60341); chr4:42153473-42153499/SHISA3 (−246370), SLC30A9 (+160963); chr4:42401197-42401268/SHISA3
(+1377), ATP8A1 (+257889); chr4:44449570-44449652/KCTD8 (+1213); chr4:44450347-44450386/KCTD8 (+45
7); chr4:47839623-47839675/CORIN (+410); chr4:48343315-48343332/SLAIN2 (−289); chr4:48492619-484932
27/ZAR1 (+614); chr4:48987806-48987843/CWH43 (−440); chr4:52708986-52709038/DCUN1D4 (−264); chr4:52
904168-52904227/SGCB (+287); chr4:52904419-52904505/SGCB (+23); chr4:52942957-52943214/SPATA18
(+25493), USP46 (+582416); chr4:53525596-53525634/USP46 (−113); chr4:53578649-53578692/USP46 (−5316
9), ERVMER34-1 (+39136); chr4:53588155-53588204/USP46 (−62678), ERVMER34-1 (+29627); chr4:5495802
3-54958072/CHIC2 (−27233), GSX2 (−8200); chr4:54967964-54968025/PDGFRA (−127269), GSX2 (+1747); chr
4:54975392-54975515/PDGFRA (−119810), GSX2 (+9206); chr4:54975932-54975990/PDGFRA (−119303), GS
X2 (+9713); chr4:54976116-54976170/PDGFRA (−119121), GSX2 (+9895); chr4:55093833-55093888/PDGFRA
(−1403); chr4:55098093-55098197/KIT (−425950), PDGFRA (+2881); chr4:55523467-55523514/KIT (−604); chr
4:55991639-55991802/KDR (+41); chr4:55992090-55992152/KDR (−359); chr4:56659867-56659934/NMU (−157
436), EXOC1 (−59915); chr4:56719793-56719823/EXOC1 (−8); chr4:57302172-57302192/PPAT (−380), PAICS
(−87); chr4:57333497-57333548/SRP72 (−239); chr4:57521396-57521505/HOPX (+1237), ARL9 (+150076); chr4:
57521664-57521700/HOPX (+1006), ARL9 (+150307); chr4:57522383-57522419/HOPX (+287); chr4:6206607
1-62066133/LPHN3 (−296737); chr4:62068202-62068237/LPHN3 (−294619); chr4:62321693-62321779/LPHN3
(−41103); chr4:66535315-66535350/EPHA5 (+320); chr4:66535483-66535574/EPHA5 (+124); chr4:68566624-68
566656/UBA6 (+249); chr4:69215398-69215434/TMPRSS11B (−104004), TMPRSS11E (−97751); chr4:7175822
1-71758376/GRSF1 (−52672), MOB1B (−9758); chr4:71768508-71768550/MOB1B (+472); chr4:74735148-7473
5193/CXCL1 (+62); chr4:74864269-74864367/CXCL5 (+98); chr4:76439484-76439548/THAP6 (−138), RCHY1
(+124); chr4:77342211-77342244/SHROOM3 (−14025), STBD1 (+115049); chr4:77901550-77901649/SEPT11
(+30705), CCNI (+95525); chr4:78078131-78078154/CCNG2 (−214); chr4:79472983-79473033/ANXA3 (+266); c
hr4:81188316-81188384/FGF5 (+608); chr4:81189661-81189713/BMP3 (−762432), FGF5 (+1945); chr4:819523
12-81952363/BMP3 (+219); chr4:82135888-82135919/PRKG2 (−9690), RASGEF1B (+257157); chr4:82136205-
82136243/PRKG2 (−10010), RASGEF1B (+256837); chr4:82393087-82393128/RASGEF1B (−47); chr4:8329491
8-83294938/HNRNPD (+221); chr4:83295242-83295288/HNRNPD (−116); chr4:83350955-83351016/ENOPH1
(−740), HNRPDL (+392); chr4:83718924-83718966/TMEM150C (−235819), SCD5 (+1065); chr4:83931598-8393
1737/LIN54 (+314); chr4:83931932-83931963/LIN54 (+34); chr4:84029494-84029519/COPS4 (+73268), COQ2
(+176560); chr4:84406384-84406406/FAM175A (−105); chr4:85403928-85403980/NKX6-1 (+15433), AGPAT9
(+946297); chr4:85404141-85404224/NKX6-1 (+15204), AGPAT9 (+946526); chr4:85404476-85404649/NKX6-1
(+14824), AGPAT9 (+946906); chr4:85414114-85414148/NKX6-1 (+5256), AGPAT9 (+956474); chr4:8541434
4-85414390/NKX6-1 (+5020), AGPAT9 (+956710); chr4:85418223-85418264/NKX6-1 (+1143), AGPAT9 (+9605
87); chr4:85418370-85418521/NKX6-1 (+941); chr4:85418652-85419120/NKX6-1 (+501); chr4:85419641-8541
9661/NKX6-1 (−264); chr4:85421257-85421286/NKX6-1 (−1885); chr4:85422929-85422972/NKX6-1 (−3564); chr
4:85503911-85503985/CDS1 (−109); chr4:87856067-87856109/AFF1 (−66); chr4:89079808-89079844/ABCG2
(+185); chr4:89205873-89205908/PPM1K (−3); chr4:89378767-89378831/HERC5 (+531); chr4:89444834-89444
887/PIGY (−1921), PIGY (+94); chr4:90759056-90759130/SNCA (−743); chr4:93226704-93226728/GRID2 (+116
6); chr4:94755858-94756001/SMARCAD1 (−372829), ATOH1 (+5852); chr4:95373533-95373607/PDLIM5 (+53
2); chr4:100815225-100815264/LAMTOR3 (+458); chr4:100867744-100867774/DNAJB14 (+120); chr4:100867
923-100867945/DNAJB14 (−55); chr4:100871984-100872096/DNAJB14 (−4161), H2AFZ (−528); chr4:10271187
8-102712137/BANK1 (+244); chr4:102712396-102712522/BANK1 (+695); chr4:103681425-103681489/MANBA
(+694); chr4:106395309-106395382/PPA2 (−119); chr4:106816382-106816425/NPNT (−193); chr4:108853438-
108853490/CYP2U1 (+747); chr4:109087826-109087853/LEF1 (+2272), HADH (+176970); chr4:109571893-10
9571956/OSTC (+184); chr4:110223428-110223578/COL25A1 (+296); chr4:110624608-110624637/CASP6 (+6);
chr4:110651003-110651046/PLA2G12A (+217); chr4:111531925-111531943/PITX2 (+31345), ENPEP (+134
705); chr4:111532461-111532704/PITX2 (+30696), ENPEP (+135354); chr4:111533199-111533328/PITX2 (+3
0015), ENPEP (+136035); chr4:111536506-111536561/PITX2 (+26745), ENPEP (+139305); chr4:111536975-1
11537066/PITX2 (+26258), ENPEP (+139792); chr4:111539328-111539478/PITX2 (+23876), ENPEP (+142174);
chr4:111540024-111540186/PITX2 (+23174), ENPEP (+142876); chr4:111540644-111540782/PITX2 (+2256
6), ENPEP (+143484); chr4:111541763-111541842/PITX2 (+21476), ENPEP (+144574); chr4:111542210-1115
42228/PITX2 (+21060), ENPEP (+144990); chr4:111543346-111543403/PITX2 (+19904), ENPEP (+146146); c
hr4:111543696-111543720/PITX2 (+19571), ENPEP (+146479); chr4:111544181-111544298/PITX2 (+19039),
ENPEP (+147011); chr4:111552352-111552419/PITX2 (+10893), ENPEP (+155157); chr4:111554965-1115551
93/PITX2 (+8200), ENPEP (+157850); chr4:111558443-111558498/PITX2 (+4808), ENPEP (+161242); chr4:11
1562377-111562491/PITX2 (+845); chr4:113431855-113431915/NEUROG2 (+5443), ALPK1 (+213386); chr4:1
13432155-113432226/NEUROG2 (+5137), ALPK1 (+213692); chr4:113432504-113432518/NEUROG2 (+4817),
ALPK1 (+214012); chr4:113442186-113442246/NEUROG2 (−4888); chr4:113558407-113558466/C4orf21 (−286),
LARP7 (−175); chr4:113627576-113627681/ANK2 (−343156), LARP7 (+69017); chr4:117847286-117847306/T
RAM1L1 (+159440); chr4:117847459-117847469/TRAM1L1 (+159272); chr4:118006635-118006673/TRAM1L1
(+82); chr4:119606802-119606828/METTL14 (+241); chr4:119757346-119757364/SEC24D (−29); chr4:1197575
08-119757549/SEC24D (−203); chr4:120376029-120376092/AX746903 (−172884), FABP2 (−132745); chr4:1205
48853-120548871/AX746903 (−83); chr4:122685285-122685351/ANXA5 (−67171), TMEM155 (+1022); chr4:122
685891-122686113/TMEM155 (+338); chr4:122744662-122744711/CCNA2 (+401); chr4:123653628-12365367
9/BBS12 (−203); chr4:128554124-128554187/SLC25A31 (−97399); chr4:128700587-128700681/HSPA4L (−2819);
chr4:128886019-128886040/C4orf29 (−431); chr4:134072611-134072676/PCDH10 (+2174); chr4:134073081-
134073127/PCDH10 (+2634); chr4:135122791-135122846/PABPC4L (+84); chr4:140005253-140005291/ELF2
(+55379), CCRN4L (+68329); chr4:140201157-140201192/C4orf49 (+317); chr4:140216129-140216178/C4orf4
9 (−14662), NAA15 (−6522); chr4:140375013-140375054/RAB33B (+73); chr4:140656748-140656857/MGST2
(+69881), MAML3 (+418430); chr4:141294521-141294545/SCOC (+29936), CLGN (+54282); chr4:142142086-1
42142145/ZNF330 (+67); chr4:142558128-142558149/IL15 (+390); chr4:143767385-143767433/INPP4B (+195);
chr4:144107046-144107119/GAB1 (−150900), USP38 (+1013); chr4:144257623-144257671/GAB1 (−336); chr4:
144621440-144621514/FREM3 (+351); chr4:144621897-144621981/FREM3 (−111); chr4:147558382-1475584
76/POU4F2 (−1616); chr4:147560079-147560133/POU4F2 (+61); chr4:147560419-147560459/POU4F2 (+394);
chr4:147561146-147561359/POU4F2 (+1208), TTC29 (+305781); chr4:147561950-147561999/POU4F2 (+193
0), TTC29 (+305059); chr4:147569268-147569278/POU4F2 (+9228), TTC29 (+297761); chr4:147576202-1475
76308/POU4F2 (+16210), TTC29 (+290779); chr4:154709611-154709758/SFRP2 (+543); chr4:154710618-154
710728/SFRP2 (−445); chr4:154711333-154711469/SFRP2 (−1173); chr4:154711696-154711767/SFRP2 (−1504);
chr4:155411211-155411233/DCHS2 (−98773), PLRG1 (+60363); chr4:155411498-155411567/DCHS2 (−9908
4), PLRG1 (+60052); chr4:155663921-155663970/LRAT (−1217); chr4:155664149-155664189/LRAT (−994); chr
4:155666330-155666407/RBM46 (−36058), LRAT (+1206); chr4:156129387-156129443/NPY2R (−366); chr4:15
6297734-156297941/MAP9 (+284); chr4:157997188-157997215/GLRB (−75); chr4:158143466-158143609/GRI
A2 (+1802), FAM198B (+950664); chr4:159091853-159091899/FAM198B (+2326), GRIA2 (+950140); chr4:159
092088-159092154/FAM198B (+2081), GRIA2 (+950385); chr4:159593078-159593172/ETFDH (−152); chr4:159
593324-159593388/ETFDH (+79); chr4:160025211-160025228/RAPGEF2 (−163778), FNIP2 (+335038); chr4:1
65305024-165305059/MARCH1 (−635); chr4:165898706-165898741/TRIM61 (+94); chr4:166795993-16679611
7/TLL1 (+1645); chr4:169753506-169753535/CBR4 (+177947), PALLD (+335304); chr4:169799101-169799248/
CBR4 (+132293), PALLD (+380958); chr4:169799400-169799493/CBR4 (+132021), PALLD (+381230); chr4:1
70695989-170696065/C4orf27 (−16934), MFAP3L (+251402); chr4:170947135-170947173/MFAP3L (+275); chr
4:170948038-170948089/MFAP3L (−635); chr4:171011576-171011647/AADAT (−429); chr4:172734148-172734
188/GALNTL6 (−407); chr4:172734425-172734444/GALNTL6 (−140); chr4:172734860-172735016/GALNTL6
(+363); chr4:173290883-173290935/GALNT7 (−798995), GALNTL6 (+556334); chr4:174422216-174422297/SCR
G1 (−101640), HAND2 (+29121); chr4:174427945-174428046/SCRG1 (−107379), HAND2 (+23382); chr4:17442
8327-174428428/SCRG1 (−107761), HAND2 (+23000); chr4:174429867-174429949/SCRG1 (−109291), HAND2
(+21470); chr4:174430092-174430140/SCRG1 (−109499), HAND2 (+21262); chr4:174432306-174432379/SCR
G1 (−111726), HAND2 (+19035); chr4:174437891-174437937/SCRG1 (−117297), HAND2 (+13464); chr4:17443
9003-174439053/SCRG1 (−118411), HAND2 (+12350); chr4:174442994-174443044/SCRG1 (−122402), HAND2
(+8359); chr4:174444073-174444198/SCRG1 (−123519), HAND2 (+7242); chr4:174445205-174445321/SCRG
1 (−124646), HAND2 (+6115); chr4:174446371-174446507/SCRG1 (−125822), HAND2 (+4939); chr4:17444835
3-174448460/SCRG1 (−127790), HAND2 (+2971); chr4:174450727-174450751/HAND2 (+639); chr4:174452408-
174452460/HAND2 (−1056); chr4:174459375-174459527/CEP44 (−745604), HAND2 (−8073); chr4:176922668-
176922729/GPM6A (+949); chr4:176986786-176986855/GPM6A (−63173), SPATA4 (+130001); chr4:17698718
0-176987206/GPM6A (−63545), SPATA4 (+129629); chr4:183061773-183062010/ODZ3 (−183207); chr4:18306
3973-183063994/ODZ3 (−181115); chr4:183247001-183247110/ODZ3 (+1957), DCTD (+591574); chr4:183369
558-183369579/ODZ3 (+124470), DCTD (+469061); chr4:183370292-183370318/ODZ3 (+125206), DCTD (+46
8325); chr4:183746783-183746880/DCTD (+91798), ODZ3 (+501733); chr4:184827795-184828122/STOX2 (+1
450), ENPP6 (+311155); chr4:184828385-184828423/STOX2 (+1895), ENPP6 (+310710); chr4:185395410-185
395456/IRF2 (+293); chr4:185570395-185570409/CCDC111 (−365), CASP3 (+227); chr4:185942473-18594249
1/SLC25A4 (−121935), HELT (+2399); chr4:186131059-186131101/SNX25 (−204); chr4:186131246-186131284/
SNX25 (−19); chr4:186131636-186131710/SNX25 (+389); chr4:186560307-186560338/PDLIM3 (−103611), SOR
BS2 (+45677); chr4:187113414-187113481/CYP4V2 (+774); chr4:188916495-188916565/ZFP42 (−395); chr4:1
88916749-188916864/ZFP42 (−118); chr4:188917811-188917855/ZFP42 (+908); chr4:188918090-188918127/Z
FP42 (+1184), TRIML2 (+108635); chr4:188953125-188953225/ZFP42 (+36250), TRIML2 (+73569); chr4:1892
03826-189203879/TRIML1 (+143255); chr4:189204177-189204382/TRIML1 (+143682); chr4:190862258-19086
2284/FRG1 (+297); chr4:190935974-190936002/TUBB4Q (−29960), LOC100288255 (+12424); chr4:190938358-
190938429/TUBB4Q (−32366), LOC100288255 (+10018); chr4:190938561-190938675/TUBB4Q (−32590), LOC
100288255 (+9794); chr4:190938980-190939022/TUBB4Q (−32973), LOC100288255 (+9411); chr4:190940206-
190940258/TUBB4Q (−34204), LOC100288255 (+8180); chr4:190940695-190940752/TUBB4Q (−34696), LOC1
00288255 (+7688); chr5:53187-53500/PLEKHG4B (−87029); chr5:53899-54002/PLEKHG4B (−86422); chr5:159
847-160121/SDHA (−58372), PLEKHG4B (+19611); chr5:169287-169412/SDHA (−49006), PLEKHG4B (+28977);
chr5:271500-271535/PDCD6 (−218); chr5:322048-322061/AHRR (+17763), C5orf55 (+121203); chr5:322252-3
22265/AHRR (+17967), C5orf55 (+120999); chr5:322403-322478/AHRR (+18149), C5orf55 (+120817); chr5:34
3893-343915/AHRR (+39612), C5orf55 (+99354); chr5:392741-392815/C5orf55 (+50480), AHRR (+88486); chr
5:403141-403284/C5orf55 (+40045), AHRR (+98921); chr5:442701-442769/EXOC3 (−599), C5orf55 (+523); chr
5:501957-502001/SLC9A3 (+22570), EXOC3 (+58645); chr5:523803-523856/SLC9A3 (+719); chr5:525191-525
232/SLC9A3 (−663); chr5:926927-927008/NKD2 (−82200), TRIP13 (+33999); chr5:1015074-1015189/NKD2 (+5
964), SLC12A7 (+97040); chr5:1060595-1060647/NKD2 (+51453), SLC12A7 (+51551); chr5:1099574-1099610/
SLC12A7 (+12580), NKD2 (+90424); chr5:1108421-1108489/SLC12A7 (+3717), NKD2 (+99287); chr5:1108890-
1108966/SLC12A7 (+3244), NKD2 (+99760); chr5:1246253-1246316/SLC6A18 (+20815), TERT (+48877); chr
5:1295443-1295604/TERT (−362); chr5:1295724-1295758/TERT (−579); chr5:1386677-1386741/CLPTM1L (−41
707), SLC6A3 (+58834); chr5:1444763-1444851/SLC6A3 (+736); chr5:1445256-1445450/SLC6A3 (+190); chr5:
1445760-1445840/SLC6A3 (−257); chr5:1446101-1446236/SLC6A3 (−626); chr5:1446370-1446442/SLC6A3 (−8
63); chr5:1446880-1446927/SLC6A3 (−1361); chr5:1523739-1523788/LPCAT1 (+312); chr5:1523933-1523964/
LPCAT1 (+127); chr5:1633876-1633917/LPCAT1 (−109821), MRPL36 (+166059); chr5:1799461-1799500/NDU
FS6 (−2015), MRPL36 (+475); chr5:1799833-1799910/NDUFS6 (−1624), MRPL36 (+84); chr5:1851448-1851534/
IRX4 (+31389), NDUFS6 (+49995); chr5:1876307-1876637/IRX4 (+6408), NDUFS6 (+74976); chr5:1877103-1
877194/IRX4 (+5731), NDUFS6 (+75653); chr5:1878029-1878223/IRX4 (+4754), NDUFS6 (+76630); chr5:1879
662-1879689/IRX4 (+3204), NDUFS6 (+78180); chr5:1881769-1881828/IRX4 (+1081), NDUFS6 (+80303); chr5:
1882136-1882347/IRX4 (+638); chr5:1882922-1883131/IRX4 (−147); chr5:1883617-1883879/IRX4 (−868); chr5:
1886177-1886227/IRX4 (−3322); chr5:1886842-1886997/IRX4 (−4040); chr5:1887146-1887421/IRX4 (−4404); ch
r5:1887582-1887655/IRX4 (−4739); chr5:1931005-1931064/IRX4 (−48155), IRX2 (+820734); chr5:1931314-1931
352/IRX4 (−48453), IRX2 (+820436); chr5:1931577-1931617/IRX4 (−48717), IRX2 (+820172); chr5:1952639-195
2723/IRX4 (−69801), IRX2 (+799088); chr5:1952861-1952958/IRX4 (−70030), IRX2 (+798859); chr5:2001395-20
01457/IRX4 (−118546), IRX2 (+750343); chr5:2038863-2038947/IRX4 (−156025), IRX2 (+712864); chr5:273913
0-2739210/IRX4 (−856290), IRX2 (+12599); chr5:2739355-2739551/IRX4 (−856573), IRX2 (+12316); chr5:27398
03-2739860/IRX4 (−856952), IRX2 (+11937); chr5:2740301-2740430/IRX4 (−857486), IRX2 (+11403); chr5:2740
665-2741122/IRX4 (−858014), IRX2 (+10875); chr5:2743660-2743698/IRX4 (−860799), IRX2 (+8090); chr5:2748
490-2748549/C5orf38 (−3742), IRX2 (+3249); chr5:2748780-2749065/C5orf38 (−3339), IRX2 (+2846); chr5:2749
407-2749604/C5orf38 (−2756), IRX2 (+2263); chr5:2749777-2749840/C5orf38 (−2453), IRX2 (+1960); chr5:2750
770-2750965/C5orf38 (−1394), IRX2 (+901); chr5:2751404-2751454/C5orf38 (−833), IRX2 (+340); chr5:2752442-
2752508/IRX2 (−706), C5orf38 (+213); chr5:2754807-2754870/IRX2 (−3070), C5orf38 (+2577); chr5:2756603-2
756673/IRX2 (−4869), C5orf38 (+4376); chr5:3118618-3118730/IRX1 (−477494), C5orf38 (+366412); chr5:3197
894-3197991/IRX1 (−398225), C5orf38 (+445681); chr5:3590658-3590803/IRX1 (−5437), C5orf38 (+838469); ch
r5:3591552-3591613/IRX1 (−4585); chr5:3592463-3592642/IRX1 (−3615); chr5:3594520-3594585/IRX1 (−1615);
chr5:3594877-3595055/IRX1 (−1202); chr5:3595369-3595447/IRX1 (−760); chr5:3595587-3595727/IRX1 (−511);
chr5:3596191-3596555/IRX1 (+205); chr5:3596725-3596824/IRX1 (+607); chr5:3599445-3599502/IRX1 (+3306);
chr5:3599608-3600074/IRX1 (+3673); chr5:3600320-3600390/IRX1 (+4187); chr5:3600639-3600732/IRX1
(+4518); chr5:3600866-3600922/IRX1 (+4726); chr5:3602351-3602716/IRX1 (+6366); chr5:3605980-3606058/IRX
1 (+9851); chr5:3606435-3606711/IRX1 (+10405); chr5:5139621-5139753/ADAMTS16 (−756); chr5:5140000-51
40031/ADAMTS16 (−427); chr5:5140456-5140527/ADAMTS16 (+49); chr5:5140758-5140849/ADAMTS16 (+361);
chr5:5887156-5887231/MED10 (+491445), ADAMTS16 (+746751); chr5:5887344-5887387/MED10 (+491273),
ADAMTS16 (+746923); chr5:6632482-6632610/SRD5A1 (−954), NSUN2 (+927); chr5:6714663-6714707/PAP
D7 (−33); chr5:7395079-7395160/ADCY2 (−1223); chr5:7395394-7395433/ADCY2 (−929); chr5:7849835-784994
5/FASTKD3 (+19260), ADCY2 (+453547); chr5:7851384-7851409/FASTKD3 (+17753), ADCY2 (+455054); chr5:
10333158-10333224/CMBL (−25023), MARCH6 (−20637); chr5:10442305-10442372/ROPN1L (+365); chr5:105
64659-10564800/ROPN1L (+122756), DAP (+196657); chr5:10565228-10565262/ROPN1L (+123271), DAP (+1
96142); chr5:11384580-11384964/DAP (−623385), CTNND2 (+519338); chr5:11903725-11903821/CTNND2 (+3
37); chr5:11904174-11904195/CTNND2 (−75); chr5:11904380-11904455/CTNND2 (−308); chr5:14144105-1414
4285/TRIO (+366); chr5:14144958-14144980/TRIO (+1140), ANKH (+726918); chr5:15500689-15500735/FBXL
7 (+407); chr5:16179153-16179266/MARCH11 (+687); chr5:16179407-16179554/MARCH11 (+416); chr5:1617
9800-16179942/MARCH11 (+26); chr5:16180162-16180182/MARCH11 (−275); chr5:16180321-16180371/MAR
CH11 (−449); chr5:17217865-17217991/BASP1 (+178); chr5:17218934-17218985/BASP1 (+1210); chr5:172192
82-17219325/BASP1 (+1554); chr5:31194291-31194311/CDH6 (+539); chr5:31194695-31194760/CDH6 (+966);
chr5:31639088-31639164/PDZD2 (−159870), DROSHA (−106844); chr5:32313142-32313198/MTMR12 (−56); c
hr5:32712102-32712289/NPR3 (+758); chr5:32713577-32713744/TARS (−727237), NPR3 (+2223); chr5:33936
945-33936969/RXFP3 (+466); chr5:34915405-34915469/BRIX1 (−383), RAD1 (+343); chr5:35925899-35925952/
CAPSL (+12955), IL7R (+68935); chr5:36066909-36066939/UGT3A2 (+99); chr5:37834737-37835014/NUP155
(−463679), GDNF (+1053); chr5:37835350-37835441/GDNF (+533); chr5:37839736-37839807/GDNF (−3843);
chr5:37840220-37840267/GDNF (−4315); chr5:37840460-37840479/GDNF (−4541); chr5:38257909-38257944/E
GFLAM (−584); chr5:38467507-38467658/LIFR (+127924), EGFLAM (+209072); chr5:38556361-38556411/LIFR
(+39121), EGFLAM (+297875); chr5:38556674-38556773/LIFR (+38783), EGFLAM (+298213); chr5:38846165-
38846468/OSMR (+216); chr5:38846840-38846966/OSMR (+802); chr5:40681228-40681261/PTGER4 (+1213),
TTC33 (+74827); chr5:40681875-40681954/PTGER4 (+1883), TTC33 (+74157); chr5:41637483-41637613/PL
CXD3 (−126818), OXCT1 (+233243); chr5:41904703-41904762/OXCT1 (−33942), FBXO4 (−20623); chr5:419258
07-41925876/FBXO4 (+486); chr5:42423530-42423573/GHR (−325); chr5:42944317-42944377/SEPP1 (−13232
3), C5orf39 (+96100); chr5:42951312-42951419/SEPP1 (−139342), C5orf39 (+89081); chr5:42952164-4295246
1/SEPP1 (−140289), C5orf39 (+88134); chr5:42952604-42952683/SEPP1 (−140620), C5orf39 (+87803); chr5:42
992242-42992375/SEPP1 (−180285), C5orf39 (+48138); chr5:42992598-42992782/SEPP1 (−180666), C5orf39
(+47757); chr5:42994983-42995072/SEPP1 (−183004), C5orf39 (+45419); chr5:43000639-43000675/SEPP1 (−1
88633), C5orf39 (+39790); chr5:43018177-43018326/SEPP1 (−206228), C5orf39 (+22195); chr5:43039678-430
39708/C5orf39 (+754); chr5:43556777-43556875/PAIP1 (+369); chr5:44389609-44389781/FGF10 (−911); chr5:
45695240-45695313/HCN1 (+943); chr5:45695608-45695648/HCN1 (+592); chr5:45695964-45696137/HCN1
(+169); chr5:49736978-49737098/EMB (+196); chr5:50258952-50259012/ISL1 (−419976), PARP8 (+296210); ch
r5:50265166-50265190/ISL1 (−413780), PARP8 (+302406); chr5:50265672-50265720/ISL1 (−413262), PARP8
(+302924); chr5:50683846-50683913/ISL1 (+4922); chr5:50685355-50685653/ISL1 (+6546); chr5:50685796-506
85846/ISL1 (+6863); chr5:50695298-50695350/ISL1 (+16366); chr5:53606372-53606415/ARL15 (+9); chr5:538
14373-53814453/SNX18 (+820); chr5:54179409-54179429/ESM1 (+101995), SNX18 (+365826); chr5:5417955
8-54179609/ESM1 (+101830), SNX18 (+365991); chr5:54468808-54468870/GPX8 (+12855), IDAS (+54304); c
hr5:54516681-54516831/IDAS (+6387), GPX8 (+60772); chr5:54519289-54519382/IDAS (+3807), GPX8 (+633
52); chr5:54527167-54527322/IDAS (−4102); chr5:54527636-54527844/IDAS (−4597); chr5:54831048-54831085/
PPAP2A (−194); chr5:55117662-55117821/IL31RA (−29465), DDX4 (+83897); chr5:55885079-55885214/IL6ST
(−594326), MAP3K1 (−225753); chr5:56247744-56247777/MIER3 (+193); chr5:59105924-59105973/PDE4D (+8
3672); chr5:59188212-59188275/PDE4D (+1377); chr5:60241477-60241550/ERCC8 (−609), NDUFAF2 (+558);
chr5:60921604-60921662/C5orf64 (−12003), ZSWIM6 (+293533); chr5:60921839-60922050/C5orf64 (−11691),
ZSWIM6 (+293845); chr5:60922295-60922459/C5orf64 (−11259), ZSWIM6 (+294277); chr5:63255243-6325531
5/HTR1A (+2267); chr5:63257019-63257086/HTR1A (+493); chr5:63801781-63801804/RGS7BP (−659); chr5:6
3802305-63802339/RGS7BP (−130); chr5:63986528-63986569/FAM159B (+414); chr5:65891980-65892018/MA
ST4 (−177); chr5:67512205-67512261/PIK3R1 (+649); chr5:68513626-68513675/MRPS36 (+78); chr5:6871114
7-68711333/MARVELD2 (+301); chr5:68711450-68711489/MARVELD2 (+531); chr5:70363417-70363470/GTF
2H2 (+53); chr5:71014979-71015031/CARTPT (+15); chr5:72112362-72112410/ZNF366 (−309137), TNPO1 (−3
1544); chr5:72415923-72416022/TMEM171 (−415); chr5:72511748-72511765/TMEM174 (+42734), FOXD1 (+2
32595); chr5:72526066-72526136/TMEM174 (+57078), FOXD1 (+218251); chr5:72526415-72526491/TMEM17
4 (+57430), FOXD1 (+217899); chr5:72528110-72528189/TMEM174 (+59127), FOXD1 (+216202); chr5:725298
26-72529889/TMEM174 (+60835), FOXD1 (+214494); chr5:72594127-72594267/TMEM174 (+125174), FOXD1
(+150155); chr5:72594382-72594494/TMEM174 (+125415), FOXD1 (+149914); chr5:72594837-72594867/TM
EM174 (+125829), FOXD1 (+149500); chr5:72595017-72595046/TMEM174 (+126009), FOXD1 (+149320); chr
5:72595722-72595773/TMEM174 (+126725), FOXD1 (+148604); chr5:72598983-72599059/TMEM174 (+12999
8), FOXD1 (+145331); chr5:72599442-72599462/TMEM174 (+130429), FOXD1 (+144900); chr5:72677353-726
77649/FOXD1 (+66851), TMEM174 (+208478); chr5:72677826-72677997/FOXD1 (+66440), TMEM174 (+2088
89); chr5:72678281-72678352/FOXD1 (+66035), TMEM174 (+209294); chr5:72715348-72715590/FOXD1 (+28
883), TMEM174 (+246446); chr5:72715696-72715730/FOXD1 (+28639), TMEM174 (+246690); chr5:72731960-
72732016/FOXD1 (+12364), TMEM174 (+262965); chr5:72732713-72732869/FOXD1 (+11561), TMEM174 (+2
63768); chr5:72746684-72746729/FOXD1 (−2355); chr5:73935988-73936032/ENC1 (+1239); chr5:74348895-74
348906/HMGCR (−284092), GCNT4 (−22177); chr5:74632624-74632654/HMGCR (−354); chr5:75377800-75377
859/SV2C (−1475); chr5:76114396-76114443/F2RL1 (−413); chr5:76115773-76115809/F2RL1 (+958); chr5:762
49671-76249695/AGGF1 (−76527), CRHBP (+1003); chr5:76249907-76249944/AGGF1 (−76284), CRHBP (+12
46); chr5:76373468-76373514/ZBED3 (+9539), AGGF1 (+47281); chr5:76924024-76924086/OTP (+10467), PD
E8B (+417349); chr5:76924300-76924416/OTP (+10164), PDE8B (+417652); chr5:76933266-76933464/OTP
(+1157), PDE8B (+426659); chr5:76934654-76934676/OTP (−143); chr5:76937423-76937510/OTP (−2945); chr5:
76939219-76939435/OTP (−4805); chr5:76941498-76941512/OTP (−6983), TBCA (+130680); chr5:77072139-77
072279/TBCA (−24); chr5:77140805-77140867/TBCA (−68651), AP3B1 (+449692); chr5:77253511-77253595/T
BCA (−181368), AP3B1 (+336975); chr5:77253868-77253986/TBCA (−181742), AP3B1 (+336601); chr5:772692
38-77269263/TBCA (−197066), AP3B1 (+321277); chr5:77589859-77589930/AP3B1 (+633); chr5:77657214-77
657286/SCAMP1 (+911); chr5:77805898-77805966/LHFPL2 (+138716), SCAMP1 (+149593); chr5:78279886-7
8279959/LHFPL2 (−335275), ARSB (+2434); chr5:78365254-78365502/BHMT2 (−169), DMGDH (+71); chr5:783
65624-78365661/DMGDH (−194), BHMT2 (+96); chr5:78407951-78408016/BHMT (+380); chr5:78408275-7840
8297/BHMT (+682); chr5:79331337-79331476/THBS4 (+237); chr5:79865445-79865522/ANKRD34B (+820); ch
r5:79865703-79865744/ANKRD34B (+580); chr5:79865955-79865994/ANKRD34B (+329); chr5:79950171-7995
0247/MTRNR2L2 (−3355), MSH3 (−85), DHFR (+591); chr5:79950637-79950703/MTRNR2L2 (−3816), DHFR (+1
30), MSH3 (+376); chr5:80256078-80256086/RASGRF2 (−426); chr5:80256370-80256428/RASGRF2 (−109); ch
r5:80256808-80256887/RASGRF2 (+340); chr5:80529120-80529162/CKMT2 (+2); chr5:80597287-80597322/Z
CCHC9 (−97); chr5:80689795-80689879/ACOT12 (+151); chr5:81045632-81045675/ACOT12 (−355666), SSBP
2 (+1418); chr5:81047476-81047532/SSBP2 (−432); chr5:83680665-83680693/EDIL3 (−68); chr5:87440120-874
40140/CCNH (−731280), TMEM161B (+124535); chr5:87440271-87440313/CCNH (−731442), TMEM161B (+124
373); chr5:87955665-87955839/TMEM161B (−391087), MEF2C (+244170); chr5:87956663-87956679/TMEM16
1B (−392006), MEF2C (+243251); chr5:87968686-87968772/TMEM161B (−404064), MEF2C (+231193); chr5:92
906372-92906455/NR2F1 (−12629); chr5:92906565-92906681/NR2F1 (−12420); chr5:92907994-92908050/NR2
F1 (−11021); chr5:92908189-92908391/NR2F1 (−10753); chr5:92908640-92908770/NR2F1 (−10338); chr5:9291
5047-92915099/NR2F1 (−3970); chr5:92923383-92923469/NR2F1 (+4383), POU5F2 (+153883); chr5:92923716-
92923992/NR2F1 (+4811), POU5F2 (+153455); chr5:92930704-92930781/NR2F1 (+11700), POU5F2 (+14656
6); chr5:92932669-92932926/NR2F1 (+13755), POU5F2 (+144511); chr5:92934430-92934478/NR2F1 (+15411),
POU5F2 (+142855); chr5:92938012-92938365/NR2F1 (+19146), POU5F2 (+139120); chr5:93446909-934469
62/FAM172A (+468); chr5:95297416-95297447/ELL2 (+343); chr5:100239120-100239134/ST8SIA4 (−140); chr5:
100240145-100240232/ST8SIA4 (−1202); chr5:102090549-102090600/SLCO6A1 (−255855), PAM (−110952); c
hr5:107004952-107005046/EFNA5 (+1597); chr5:108085256-108085316/FER (+1763), PJA2 (+660389); chr5:1
10074216-110074272/SLC25A46 (−510); chr5:110406444-110406532/TSLP (−902); chr5:110427862-11042791
8/WDR36 (+20); chr5:110560119-110560138/CAMK4 (+182); chr5:111093425-111093474/STARD4 (−245293),
NREP (+219178); chr5:111496500-111496549/NREP (−183897), EPB41L4A (+258485); chr5:111496958-11149
7007/NREP (−184355), EPB41L4A (+258027); chr5:112043772-112043800/EPB41L4A (−288776), APC (−29770);
chr5:113698264-113698330/KCNN2 (+281); chr5:113698584-113698669/KCNN2 (+611); chr5:113698844-11
3698951/KCNN2 (+882); chr5:114193690-114193762/TRIM36 (+322517), KCNN2 (+495710); chr5:114514825-
114515009/TRIM36 (+1326), KCNN2 (+816901); chr5:114515580-114515610/TRIM36 (+648); chr5:114515728-
114515769/TRIM36 (+494); chr5:114879580-114879637/FEM1C (+982); chr5:115151358-115151649/CDO1
(+901); chr5:115152385-115152491/CDO1 (−33); chr5:115297293-115297376/AQPEP (−816); chr5:115297986-11
5298204/AQPEP (−56); chr5:115298582-115298781/AQPEP (+531); chr5:121297445-121297504/SRFBP1 (−18
1); chr5:122422315-122422508/PRDM6 (−2429); chr5:122423432-122423573/PRDM6 (−1338); chr5:122425641-
122425701/PRDM6 (+830); chr5:122425956-122426004/PRDM6 (+1139), CEP120 (+333306); chr5:12242670
7-122426796/PRDM6 (+1911), CEP120 (+332534); chr5:122433905-122434262/PRDM6 (+9243), CEP120 (+3
25202); chr5:122434528-122434605/PRDM6 (+9726), CEP120 (+324719); chr5:122435163-122435201/PRDM
6 (+10341), CEP120 (+324104); chr5:122435303-122435413/PRDM6 (+10517), CEP120 (+323928); chr5:1224
35535-122435628/PRDM6 (+10741), CEP120 (+323704); chr5:122621183-122621308/CEP120 (+138040), PR
DM6 (+196405); chr5:122759492-122759518/CEP120 (−219); chr5:126408755-126408818/C5orf63 (+397); chr5:
127872738-127872819/FBN2 (+956); chr5:127873180-127873396/FBN2 (+447); chr5:128300695-128300712/
SLC27A6 (−506); chr5:128300874-128300900/SLC27A6 (−323); chr5:129240935-129240962/CHSY3 (+426); chr
5:131131944-131132056/RAPGEF6 (−161071), ACSL6 (+215355); chr5:131563531-131563558/P4HA2 (+11); c
hr5:131593412-131593472/PDLIM4 (+91); chr5:131630561-131630593/SLC22A4 (+432); chr5:131832433-131
832488/IRF1 (−5996), IL5 (+46753); chr5:132083441-132083471/CCNI2 (+319); chr5:132147925-132147995/S
EPT8 (−34893), SHROOM1 (+14042); chr5:132148933-132148947/SEPT8 (−35873), SHROOM1 (+13062); chr5:
132149780-132149912/SEPT8 (−36779), SHROOM1 (+12156); chr5:132150223-132150248/SEPT8 (−37169),
SHROOM1 (+11766); chr5:132158547-132158771/SEPT8 (−45592), SHROOM1 (+3343); chr5:132161118-1321
61483/SHROOM1 (+701); chr5:132161711-132161807/SHROOM1 (+243); chr5:132166250-132166278/SHRO
OM1 (−4262); chr5:132202143-132202173/GDF9 (−1681), UQCRQ (−161); chr5:132362050-132362099/ZCCHC
10 (+165); chr5:133304067-133304123/C5orf15 (+311); chr5:133449335-133449376/TCF7 (−1046); chr5:13351
2118-133512160/SKP1 (+585); chr5:133707557-133707618/CDKL3 (−4823), UBE2B (+718); chr5:133984051-1
33984104/SEC24A (−397); chr5:134094985-134095038/DDX46 (+551); chr5:134363127-134363613/PITX1 (+6
594), CATSPER3 (+59774); chr5:134363827-134364128/PITX1 (+5986), CATSPER3 (+60382); chr5:13436450
4-134364573/PITX1 (+5425), CATSPER3 (+60943); chr5:134364881-134364967/PITX1 (+5040), CATSPER3
(+61328); chr5:134367266-134367284/PITX1 (+2689), CATSPER3 (+63679); chr5:134370453-134370508/PITX
1 (−517); chr5:134373713-134373776/PITX1 (−3781); chr5:134374506-134374791/PITX1 (−4685); chr5:1343765
57-134376731/PITX1 (−6680), H2AFY (+358284); chr5:134386168-134386184/PITX1 (−16212), H2AFY (+34875
2); chr5:134802500-134802534/C5orf20 (−19479), NEUROG1 (+69122); chr5:134825106-134825179/C5orf20
(−42105), NEUROG1 (+46496); chr5:134825817-134825896/C5orf20 (−42819), NEUROG1 (+45782); chr5:13482
6260-134826479/C5orf20 (−43332), NEUROG1 (+45269); chr5:134827285-134827472/C5orf20 (−44341), NEUR
OG1 (+44260); chr5:134870927-134871181/NEUROG1 (+585); chr5:134871325-134871417/NEUROG1 (+268);
chr5:134872609-134872658/NEUROG1 (−995); chr5:134880023-134880048/NEUROG1 (−8397), CXCL14 (+3
4933); chr5:135265860-135265919/IL9 (−34374), LECT2 (+24833); chr5:135266134-135266515/IL9 (−34809), L
ECT2 (+24398); chr5:136834189-136834289/SPOCK1 (+779); chr5:136834664-136834706/SPOCK1 (+333); c
hr5:136834881-136834912/SPOCK1 (+121); chr5:137090010-137090063/HNRNPA0 (+2); chr5:137090648-137
090698/HNRNPA0 (−634); chr5:137225014-137225091/PKD2L2 (−72); chr5:137513960-137513999/KIF20A (−43
7), BRD8 (+378); chr5:137549085-137549135/CDC23 (−78); chr5:137610257-137610301/GFRA3 (−26); chr5:13
7795169-137795219/EGR1 (−5987), REEP2 (+20418); chr5:138727908-138727987/MZB1 (−2343); chr5:138729
681-138729700/MZB1 (−4086); chr5:138729814-138730050/MZB1 (−4327); chr5:138850540-138850605/ECSC
R (−8253), TMEM173 (+11770); chr5:139038107-139038230/PSD2 (−137237), CXXC5 (+9868); chr5:13904973
3-139049781/PSD2 (−125649), CXXC5 (+21456); chr5:139089678-139089811/PSD2 (−85661), CXXC5 (+61444);
chr5:139128153-139128219/PSD2 (−47220), CXXC5 (+99885); chr5:139144057-139144114/PSD2 (−31320),
CXXC5 (+115785); chr5:139145148-139145195/PSD2 (−30234), CXXC5 (+116871); chr5:139175877-13917592
9/PSD2 (+497); chr5:139227624-139227704/PSD2 (+52258), NRG2 (+195220); chr5:139525948-139526053/IG
IP (+20480), PFDN1 (+156688); chr5:140012022-140012125/CD14 (+961); chr5:140012293-140012364/CD14
(+706); chr5:140026762-140026831/IK (−587), NDUFA2 (+573); chr5:140174840-140174937/PCDHAC2 (−17085
8), ZMAT2 (+94857); chr5:140201463-140201495/PCDHAC2 (−144268), ZMAT2 (+121447); chr5:140255093-1
40255158/PCDHAC2 (−90621), ZMAT2 (+175094); chr5:140256762-140256790/PCDHAC2 (−88971), ZMAT2
(+176744); chr5:140306204-140306236/PCDHAC2 (−39527), ZMAT2 (+226188); chr5:140306620-140306674/PC
DHAC2 (−39100), ZMAT2 (+226615); chr5:140306787-140307010/PCDHAC2 (−38848), ZMAT2 (+226867); chr5:
140474300-140474330/PCDHB2 (+78); chr5:140517295-140517346/PCDHB6 (−12518), PCDHB5 (+2521); chr
5:140531504-140531532/PCDHB17 (−4062); chr5:140614329-140614383/PCDHB18 (+418); chr5:140615816-1
40615861/PCDHB15 (−9308), PCDHB18 (+1901); chr5:140625017-140625088/PCDHB15 (−94); chr5:14071035
7-140710436/PCDHGC5 (−158411), TAF7 (−10046); chr5:140718631-140718773/PCDHGC5 (−150106), TAF7
(−18351); chr5:140723683-140723731/PCDHGC5 (−145101), TAF7 (−23356); chr5:140731143-140731239/PCDH
GC5 (−137617), TAF7 (−30840); chr5:140734850-140734930/PCDHGC5 (−133918), TAF7 (−34539); chr5:14073
9801-140739851/PCDHGC5 (−128982), TAF7 (−39475); chr5:140744003-140744075/PCDHGC5 (−124769), TAF
7 (−43688); chr5:140744471-140744520/PCDHGC5 (−124312), TAF7 (−44145); chr5:140762644-140762703/PC
DHGC5 (−106134), TAF7 (−62323); chr5:140773098-140773155/PCDHGC5 (−95681), TAF7 (−72776); chr5:1407
78297-140778362/PCDHGC5 (−90478), TAF7 (−77979); chr5:140782687-140782778/PCDHGC5 (−86075), TAF7
(−82382); chr5:140787473-140787622/TAF7 (−87197), PCDHGC5 (−81260); chr5:140792683-140792786/TAF7
(−92384), PCDHGC5 (−76073); chr5:140792989-140793011/TAF7 (−92649), PCDHGC5 (−75808); chr5:1407934
01-140793467/TAF7 (−93083), PCDHGC5 (−75374); chr5:140797279-140797327/TAF7 (−96952), PCDHGC5 (−7
1505); chr5:140800965-140801034/TAF7 (−100649), PCDHGC5 (−67808); chr5:140810494-140810509/TAF7
(−110151), PCDHGC5 (−58306); chr5:140810984-140811135/TAF7 (−110709), PCDHGC5 (−57748); chr5:140856
459-140856546/TAF7 (−156152), PCDHGC5 (−12305); chr5:140864527-140864611/PCDHGC5 (−4239); chr5:14
0871022-140871437/PCDHGC5 (+2422), DIAPH1 (+127392); chr5:140871987-140872153/PCDHGC5 (+3262),
DIAPH1 (+126552); chr5:140892924-140892995/PCDHGC5 (+24152), DIAPH1 (+105662); chr5:141016096-14
1016113/RELL2 (−412), HDAC3 (+318); chr5:141133242-141133302/ARAP3 (−71472), PCDH1 (+124672); chr5:
141138187-141138346/ARAP3 (−76467), PCDH1 (+119677); chr5:141153728-141153844/ARAP3 (−91986), PC
DH1 (+104158); chr5:141263143-141263260/KIAA0141 (−40183), PCDH1 (−5258); chr5:141931355-141931424/
SPRY4 (−226770), FGF1 (+134263); chr5:142782499-142782625/NR3C1 (+1483), ARHGAP26 (+632270); chr5:
142782749-142782794/NR3C1 (+1273), ARHGAP26 (+632480); chr5:145235973-145236058/SH3RF2 (−80110),
PRELID2 (−21117); chr5:145717197-145717248/POU4F3 (−1364); chr5:145718395-145718419/POU4F3 (−18
0); chr5:145719754-145719834/TCERG1 (−107079), POU4F3 (+1207); chr5:145719937-145720018/TCERG1
(−106895), POU4F3 (+1391); chr5:145722467-145722560/TCERG1 (−104359), POU4F3 (+3927); chr5:14572518
8-145725693/TCERG1 (−101432), POU4F3 (+6854); chr5:145941101-145941258/GPR151 (−45504), PPP2R2B
(+494520); chr5:146889183-146889331/DPYSL3 (+362); chr5:148033793-148033820/ADRB2 (−172349), HTR4
(−17183); chr5:148620095-148620228/AFAP1L1 (−31239), ABLIM3 (+99108); chr5:148802147-148802246/IL17
B (−43359), CSNK1A1 (+128810); chr5:149109746-149109802/PPARGC1B (−41); chr5:149461436-149461530/
TIGD6 (−80753), CSF1R (+31452); chr5:149546388-149546429/CDX1 (+65); chr5:149546603-149546648/CDX
1 (+282); chr5:149589025-149589167/SLC6A7 (+19576), CAMK2A (+80307); chr5:149682792-149682867/ARS
I (−305); chr5:149792111-149792156/CD74 (+365); chr5:149865962-149866009/RPS14 (−36667), NDST1 (−216
88); chr5:150080379-150080559/RBM22 (+200); chr5:150158205-150158234/C5orf62 (+712); chr5:150158781-
150158831/IRGM (−67279), C5orf62 (+1298); chr5:150226251-150226474/IRGM (+278); chr5:150460749-1504
60814/TNIP1 (+5965), GPX3 (+60783); chr5:150537062-150537108/ANXA6 (+358); chr5:150603552-15060358
9/ANXA6 (−66128), GM2A (−29042); chr5:151150228-151150270/G3BP1 (−1227); chr5:151304639-151304708/
GLRA1 (−277); chr5:153853304-153853386/HAND1 (+4479), SAP30L (+27828); chr5:153855411-153855657/H
AND1 (+2290), SAP30L (+30017); chr5:153855927-153856148/HAND1 (+1786), SAP30L (+30521); chr5:15385
7198-153857284/HAND1 (+583); chr5:153858895-153858916/HAND1 (−1082); chr5:153862180-153862218/HA
ND1 (−4375); chr5:153873055-153873085/LARP1 (−219392), HAND1 (−15246); chr5:154291297-154291417/GE
MIN5 (+26419), CNOT8 (+53158); chr5:155108084-155108160/SGCD (−645645), KIF4B (+714862); chr5:15510
8268-155108355/SGCD (−645455), KIF4B (+715052); chr5:155109104-155109188/SGCD (−644621), KIF4B (+7
15886); chr5:157098855-157098904/THG1L (−59443), SOX30 (−19452); chr5:158478466-158478512/EBF1 (+4
8299); chr5:158524532-158524640/EBF1 (+2202); chr5:158531993-158532073/EBF1 (−5245), RNF145 (+1028
01); chr5:158690346-158690379/UBLCP1 (+274); chr5:158690530-158690602/UBLCP1 (+477); chr5:15873761
7-158737662/IL12B (+19841), UBLCP1 (+47551); chr5:159399165-159399375/TTC1 (−36910), ADRA1B (+555
30); chr5:159436043-159436105/TTC1 (−106); chr5:160974514-160974540/GABRB2 (+603); chr5:160975934-1
60975955/GABRB2 (−815); chr5:161275152-161275346/GABRA1 (−2143); chr5:167956267-167956413/FBLL1
(−242); chr5:167956823-167956844/FBLL1 (+252); chr5:168727101-168727164/PANK3 (−720519), SLIT3 (+100
0); chr5:168727928-168727994/SLIT3 (+172); chr5:168728728-168728889/SLIT3 (−676); chr5:169729914-1697
29981/KCNIP1 (−50933), LCP2 (−5126); chr5:169931171-169931223/GABRP (−279526), KCNMB1 (−114559); ch
r5:169931327-169931353/GABRP (−279383), KCNMB1 (−114702); chr5:169931542-169931584/GABRP (−2791
60), KCNMB1 (−114925); chr5:170735321-170735684/TLX3 (−785); chr5:170736164-170736715/TLX3 (+152); c
hr5:170736831-170736897/TLX3 (+576); chr5:170737032-170737168/TLX3 (+812); chr5:170737864-17073793
5/NPM1 (−76808), TLX3 (+1612); chr5:170738690-170738823/NPM1 (−75951), TLX3 (+2469); chr5:170740478-
170740574/NPM1 (−74182), TLX3 (+4238); chr5:170741325-170741358/NPM1 (−73366), TLX3 (+5054); chr5:17
0742276-170742386/NPM1 (−72377), TLX3 (+6043); chr5:170742600-170742672/NPM1 (−72072), TLX3 (+6348);
chr5:170743480-170743646/NPM1 (−71145), TLX3 (+7275); chr5:170847488-170847602/FGF18 (+878); chr5:
170877802-170877839/FGF18 (+31154), FBXW11 (+556056); chr5:170877961-170878093/FGF18 (+31360),
FBXW11 (+555850); chr5:170878233-170878251/FGF18 (+31575), FBXW11 (+555635); chr5:171433895-1714
33957/FBXW11 (−49); chr5:171605336-171605413/FBXW11 (−171498), STK10 (+9971); chr5:171614516-1716
14531/STK10 (+822); chr5:171710003-171710067/UBTD2 (+760); chr5:172068150-172068212/NEURL1B (−95);
chr5:172300081-172300114/RPL26L1 (−86341), ERGIC1 (+38875); chr5:172660026-172660141/NKX2-5 (+22
31), BNIP1 (+88639); chr5:172661001-172661126/NKX2-5 (+1251), BNIP1 (+89619); chr5:172663242-1726633
60/NKX2-5 (−986); chr5:172671163-172671245/NKX2-5 (−8889), STC2 (+85302); chr5:172671482-172671639/
NKX2-5 (−9246), STC2 (+84945); chr5:172672893-172672960/NKX2-5 (−10612), STC2 (+83579); chr5:1727549
63-172754985/NKX2-5 (−92659), STC2 (+1532); chr5:172755378-172755420/NKX2-5 (−93084), STC2 (+1107);
chr5:172755563-172755594/STC2 (+927); chr5:173738744-173738806/MSX2 (−412800), HMP19 (+266051); ch
r5:174035992-174036062/MSX2 (−115548), HMP19 (+563303); chr5:174148765-174148842/MSX2 (−2771); chr
5:174154369-174154455/MSX2 (+2837), DRD1 (+716751); chr5:174158688-174159006/MSX2 (+7272), DRD1
(+712316); chr5:174220817-174220856/MSX2 (+69262), DRD1 (+650326); chr5:175085315-175085524/HRH2
(+380); chr5:175223748-175223835/CPLX2 (−74709), HRH2 (+138752); chr5:175224329-175224347/CPLX2
(−74163), HRH2 (+139298); chr5:175300171-175300216/CPLX2 (+1693), THOC3 (+95351); chr5:175560220-175
560258/THOC3 (−164694), KIAA1191 (+228570); chr5:175792932-175792997/KIAA1191 (−4156), ARL10 (+463);
chr5:175815442-175815514/HIGD2A (−306), NOP16 (+62); chr5:175964851-175964918/RNF44 (−464); chr5:
176024477-176024491/GPRIN1 (+12647), CDHR2 (+48141); chr5:176047319-176047328/GPRIN1 (−10193), S
NCB (+10233); chr5:176107485-176107517/UNC5A (−130059), TSPAN17 (+33113); chr5:176170621-17617070
3/UNC5A (−66898), TSPAN17 (+96274); chr5:176236998-176237148/UNC5A (−487); chr5:176238303-1762383
52/UNC5A (+768); chr5:176543900-176544052/NSD1 (−16857), FGFR4 (+30055); chr5:176778671-176778715/
LMAN2 (+192); chr5:176784964-176785051/RGS14 (+164); chr5:176789472-176789547/SLC34A1 (−21922), R
GS14 (+4666); chr5:176830142-176830181/PFN3 (−2525); chr5:176883428-176883493/PRR7 (+8135), DBN1
(+17233); chr5:176924664-176924681/PDLIM7 (−71); chr5:176935237-176935308/PDLIM7 (−10671), DOK3 (+1
585); chr5:176981390-176981461/TMED9 (−37787), DDX41 (−37459); chr5:177209928-177210044/LOC728554
(−92276), B4GALT7 (+182867); chr5:177210319-177210406/LOC728554 (−91899), B4GALT7 (+183244); chr5:
177366994-177367067/PROP1 (+56212), LOC728554 (+64769); chr5:177411422-177411507/PROP1 (+11778),
LOC728554 (+109203); chr5:177434404-177434456/N4BP3 (−106126), PROP1 (−11187); chr5:177541222-17
7541299/N4BP3 (+705); chr5:177667803-177667864/AGXT2L2 (−8031), COL23A1 (+349722); chr5:177988918-
177988987/AGXT2L2 (−329150), COL23A1 (+28603); chr5:178003662-178003915/AGXT2L2 (−343986), COL2
3A1 (+13767); chr5:178004058-178004285/AGXT2L2 (−344369), COL23A1 (+13384); chr5:178004398-178004
470/AGXT2L2 (−344631), COL23A1 (+13122); chr5:178016571-178016681/COL23A1 (+930); chr5:178017007-
178017519/COL23A1 (+293); chr5:178054158-178054178/CLK4 (−114); chr5:178323139-178323183/ZFP2 (+2
45); chr5:178368122-178368414/ZNF454 (+74); chr5:178420360-178420535/GRM6 (+1676), ZNF454 (+52254);
chr5:178421423-178421676/GRM6 (+574); chr5:178421818-178422014/GRM6 (+208); chr5:178450357-1784
50406/ZNF879 (−394); chr5:178487304-178487341/ZNF354C (−284); chr5:178594053-178594123/ZNF354C
(+106481), ADAMTS2 (+178343); chr5:178770905-178771198/ADAMTS2 (+1379), ZNF354C (+283445); chr5:17
8771631-178771723/ADAMTS2 (+754); chr5:178771860-178771967/ADAMTS2 (+517); chr5:178772730-17877
2778/ADAMTS2 (−323); chr5:178957227-178957273/ADAMTS2 (−184819), RUFY1 (−20321); chr5:178957433-1
78957470/ADAMTS2 (−185021), RUFY1 (−20119); chr5:179050252-179050286/HNRNPH1 (+453); chr5:179105
251-179105316/C5orf60 (−33237), CANX (−20646); chr5:179194241-179194315/LTC4S (−26708), MAML1 (+344
27); chr5:179199279-179199406/LTC4S (−21643), MAML1 (+39492); chr5:179223753-179223813/LTC4S (+279
7), MGAT4B (+10169); chr5:179233398-179233418/MGAT4B (+544); chr5:179243754-179243798/SQSTM1 (−4
066); chr5:179285574-179285622/C5orf45 (+242); chr5:179507555-179507640/RNF130 (−8489), RASGEF1C
(+128532); chr5:179635290-179635359/RASGEF1C (+805); chr5:179635601-179635648/RASGEF1C (+505); c
hr5:179636155-179636200/RASGEF1C (−48); chr5:179779783-179779840/GFPT2 (+503); chr5:179780013-17
9780035/GFPT2 (+291); chr5:180018217-180018262/SCGB3A1 (+247); chr5:180018449-180018483/SCGB3A
1 (+21); chr5:180072441-180072533/SCGB3A1 (−54000), FLT4 (+4137); chr5:180075858-180076053/FLT4 (+6
68); chr5:180076458-180076532/FLT4 (+129); chr5:180100557-180100616/FLT4 (−23963), OR2Y1 (+66471); c
hr5:180100874-180101015/FLT4 (−24321), OR2Y1 (+66113); chr5:180101168-180101251/FLT4 (−24586), OR2
Y1 (+65848); chr5:180229397-180229432/MGAT1 (+633); chr5:180230630-180230689/MGAT1 (−612); chr5:18
0237221-180237257/MGAT1 (−7191), ZFP62 (+51047); chr5:180238052-180238091/MGAT1 (−8024), ZFP62
(+50214); chr5:180486329-180486371/OR2V2 (−95593), BTNL9 (+19125); chr5:180486557-180486582/OR2V2
(−95373), BTNL9 (+19345); chr5:180486782-180486864/OR2V2 (−95120), BTNL9 (+19598); chr5:180511971-180
512015/OR2V2 (−69950), BTNL9 (+44768); chr5:180524093-180524130/OR2V2 (−57831), BTNL9 (+56887); chr
5:180524258-180524395/OR2V2 (−57616), BTNL9 (+57102); chr5:180524885-180525021/OR2V2 (−56990), BT
NL9 (+57728); chr5:180527699-180527793/OR2V2 (−54197), BTNL9 (+60521); chr5:180528371-180528426/O
R2V2 (−53544), BTNL9 (+61174); chr5:180528698-180528764/OR2V2 (−53212), BTNL9 (+61506); chr5:180529
095-180529134/OR2V2 (−52828), BTNL9 (+61890); chr5:180529258-1 80529284/OR2V2 (−52672), BTNL9 (+62
046); chr5:180591785-180591827/OR2V2 (+9863), TRIM7 (+40371); chr5:180597041-180597086/OR2V2 (+15
121), TRIM7 (+35113); chr5:180597641-180597675/OR2V2 (+15715), TRIM7 (+34519); chr5:180610212-18061
0299/TRIM7 (+21921), OR2V2 (+28313); chr5:180687447-180687609/TRIM52 (+591); chr6:219979-220021/DU
SP22 (−72101); chr6:379488-379585/IRF4 (−12202), DUSP22 (+87436); chr6:391985-392152/IRF4 (+330); chr6:
392258-392322/IRF4 (+551); chr6:392434-392587/IRF4 (+772); chr6:393473-393764/IRF4 (+1880), EXOC2
(+299490); chr6:692866-692886/EXOC2 (+233); chr6:1312319-1312342/FOXQ1 (−344); chr6:1326832-1326894/F
OXF2 (−63206), FOXQ1 (+14188); chr6:1377724-1377748/FOXF2 (−12333), FOXQ1 (+65061); chr6:1378657-1
378687/FOXF2 (−11397), FOXQ1 (+65997); chr6:1378881-1379267/FOXF2 (−10995), FOXQ1 (+66399); chr6:1
383727-1383840/FOXF2 (−6285), FOXQ1 (+71109); chr6:1384180-1384625/FOXF2 (−5666), FOXQ1 (+71728);
chr6:1388493-1388556/FOXF2 (−1544); chr6:1389275-1389372/FOXF2 (−745); chr6:1390406-1390423/FOXF2
(+346); chr6:1390759-1390955/FOXF2 (+788); chr6:1393113-1393171/FOXC1 (−217539), FOXF2 (+3073); chr6:
1393384-1393442/FOXC1 (−217268), FOXF2 (+3344); chr6:1393757-1393933/FOXC1 (−216836), FOXF2 (+37
76); chr6:1394168-1394248/FOXC1 (−216473), FOXF2 (+4139); chr6:1601899-1601966/FOXC1 (−8748), FOXF
2 (+211864); chr6:1608666-1608731/FOXC1 (−1982); chr6:1612196-1612300/FOXC1 (+1567), GMDS (+63362
0); chr6:1614200-1614286/FOXC1 (+3562), GMDS (+631625); chr6:1614512-1614564/FOXC1 (+3857), GMDS
(+631330); chr6:1614713-1614933/FOXC1 (+4142), GMDS (+631045); chr6:1615082-1615242/FOXC1 (+4481),
GMDS (+630706); chr6:1615584-1615618/FOXC1 (+4920), GMDS (+630267); chr6:1620104-1620375/FOXC1
(+9559), GMDS (+625628); chr6:1624602-1624812/FOXC1 (+14026), GMDS (+621161); chr6:1904799-19048
83/FOXC1 (+294160), GMDS (+341027); chr6:3428120-3428214/TUBB2B (−200199), SLC22A23 (+28626); chr
6:4021554-4021569/PRPF4B (−7); chr6:4079168-4079225/C6orf201 (−243); chr6:5997389-5997401/NRN1 (+10
238), FARS2 (+735811); chr6:5999183-5999238/NRN1 (+8422), FARS2 (+737627); chr6:5999543-5999609/NR
N1 (+8057), FARS2 (+737992); chr6:6000139-6000350/NRN1 (+7388), FARS2 (+738661); chr6:6002420-6002
542/NRN1 (+5152), FARS2 (+740897); chr6:6003529-6003748/NRN1 (+3994), FARS2 (+742055); chr6:600389
6-6004114/NRN1 (+3628), FARS2 (+742421); chr6:6004239-6004336/NRN1 (+3345), FARS2 (+742704); chr6:
6004744-6004836/NRN1 (+2843), FARS2 (+743206); chr6:7051838-7051873/RREB1 (−56230), LY86 (+462922);
chr6:7141483-7141496/RREB1 (+33404), SSR1 (+172051); chr6:7727769-7728086/BMP6 (+917); chr6:7910
430-7910493/TXNDC5 (+585); chr6:8102675-8102745/EEF1E1 (+118); chr6:9399236-9399347/SLC35B3 (−963
498), OFCC1 (+540290); chr6:10113589-10113619/OFCC1 (−174022), TFAP2A (+301866); chr6:10381593-103
81694/OFCC1 (−442062), TFAP2A (+33826); chr6:10382116-10382284/OFCC1 (−442618), TFAP2A (+33270); c
hr6:10391216-10391324/OFCC1 (−451688), TFAP2A (+24200); chr6:10398493-10398527/OFCC1 (−458928), T
FAP2A (+16960); chr6:10404644-10404725/OFCC1 (−465103), TFAP2A (+10785); chr6:10410133-10410242/O
FCC1 (−470606), TFAP2A (+5282); chr6:10416399-10416507/TFAP2A (−983); chr6:10417530-10417835/TFAP2
A (−2213); chr6:10419440-10419476/TFAP2A (−3988); chr6:10420002-10420118/TFAP2A (−4590); chr6:104207
55-10420876/GCNT2 (−165177), TFAP2A (−5346); chr6:10421452-10421548/GCNT2 (−164493), TFAP2A (−603
0); chr6:10425478-10425629/GCNT2 (−160439), TFAP2A (−10084); chr6:10425790-10425838/GCNT2 (−160179),
TFAP2A (−10344); chr6:10720281-10720350/TMEM14C (−2832); chr6:10883023-10883114/SYCP2L (−3995),
GCM2 (−971); chr6:10883854-10883894/SYCP2L (−3190), GCM2 (−1776); chr6:11537705-11537732/TMEM170
B (−792); chr6:11538272-11538299/TMEM170B (−225); chr6:12749952-12750238/PHACTR1 (+33058), TBC1D
7 (+578692); chr6:13486569-13486607/TBC1D7 (−157801), GFOD1 (+1281); chr6:13814040-13814103/CCDC9
0A (+720); chr6:13860385-13860449/RNF182 (−65007), CCDC90A (−45625); chr6:15248710-15248748/JARID2
(+2202), DTNBP1 (+414560); chr6:16238832-16238860/GMPR (+35); chr6:17016422-17016470/RBM24 (−265
363), ATXN1 (−254725); chr6:17280852-17281118/RBM24 (−824); chr6:17393793-17393824/CAP2 (+73); chr6:
17580941-17581172/FAM8A1 (−19461), CAP2 (+187321); chr6:17600428-17600456/FAM8A1 (−76); chr6:18263
392-18263459/DEK (+1373), KDM1B (+107807); chr6:18277961-18277979/RNF144B (−109611), DEK (−13171);
chr6:19691997-19692142/ID4 (−145547); chr6:19837205-19837269/ID4 (−380); chr6:19837497-19837520/ID4
(−108); chr6:19837943-19837983/ID4 (+346); chr6:20024140-20024215/ID4 (+186561), MBOAT1 (+188492); chr
6:20024446-20024514/ID4 (+186863), MBOAT1 (+188190); chr6:20320030-20320135/MBOAT1 (−107413), E2F
3 (−82054); chr6:24646568-24646614/KIAA0319 (−208); chr6:24936033-24936143/FAM65B (−24893), CMAHP
(+201963); chr6:26020713-26020763/HIST1H1A (−2698), HIST1H4A (−1169), HIST1H3A (+20); chr6:26124617-2
6124642/HIST1H2BC (−498), HIST1H2AC (+257); chr6:26158694-26158729/HIST1H2BD (+363); chr6:2617217
0-26172268/HIST1H2BE (−11805), HIST1H2BD (+13870); chr6:26200075-26200097/HIST1H4E (−4787), HIST1
H3D (−622), HIST1H2AD (−615), HIST1H2BF (+299); chr6:26252099-26252140/HIST1H4G (−4915), HIST1H3F
(−1285), HIST1H2BH (+241); chr6:26273419-26273514/HIST1H3G (−1855), HIST1H2BI (+263); chr6:26501841-2
6501949/BTN1A1 (+437); chr6:26551059-26551083/ABT1 (−46100), HMGN4 (+12499); chr6:26597313-265973
73/ABT1 (+172); chr6:26689803-26689987/ZNF322 (−29915), HIST1H2BJ (+410680); chr6:26721194-2672124
2/ZNF322 (−61238), HIST1H2BJ (+379357); chr6:26722893-26722920/ZNF322 (−62927), HIST1H2BJ (+377668);
chr6:26723048-26723121/ZNF322 (−63105), HIST1H2BJ (+377490); chr6:26730752-26730785/ZNF322 (−707
89), HIST1H2BJ (+369806); chr6:26735118-26735203/ZNF322 (−75181), HIST1H2BJ (+365414); chr6:2675228
3-26752330/ZNF322 (−92327), HIST1H2BJ (+348268); chr6:26757530-26757579/ZNF322 (−97575), HIST1H2B
J (+343020); chr6:26766812-26766870/ZNF322 (−106861), HIST1H2BJ (+333734); chr6:26767395-26767461/Z
NF322 (−107448), HIST1H2BJ (+333147); chr6:26771302-26771344/ZNF322 (−111343), HIST1H2BJ (+329252);
chr6:26779091-26779318/ZNF322 (−119225), HIST1H2BJ (+321370); chr6:26780132-26780223/ZNF322 (−12
0198), HIST1H2BJ (+320397); chr6:26780497-26780565/ZNF322 (−120551), HIST1H2BJ (+320044); chr6:2678
0870-26780898/ZNF322 (−120904), HIST1H2BJ (+319691); chr6:27095050-27095107/ZNF322 (−435099), HIST
1H2BJ (+5496); chr6:27114417-27114459/HIST1H2AH (−470), HIST1H2BK (+199); chr6:27173547-27173632/P
RSS16 (−41912), HIST1H2AH (+58682); chr6:27203337-27203363/PRSS16 (−12152), HIST1H2AH (+88442); ch
r6:27205837-27205913/PRSS16 (−9627), HIST1H2AH (+90967); chr6:27228187-27228289/FKSG83 (−64302),
PRSS16 (+12736); chr6:27251509-27251857/FKSG83 (−40857), PRSS16 (+36181); chr6:27263314-27263398/
FKSG83 (−29184), PRSS16 (+47854); chr6:27463910-27463960/ZNF184 (−23038), HIST1H2BL (+311774); chr
6:27512884-27512994/ZNF184 (−72042), HIST1H2BL (+262770); chr6:27647736-27647890/ZNF184 (−206916),
HIST1H2BL (+127896); chr6:27782252-27782334/HIST1H2BM (−529), HIST1H2AJ (+225); chr6:27783053-277
83114/HIST1H2AJ (−566), HIST1H2BM (+262); chr6:27805907-27805936/HIST1H2BN (−518), HIST1H2AK (+19
5); chr6:27834863-27834904/HIST1H1B (+475); chr6:27835097-27835377/HIST1H1B (+122); chr6:28219650-2
8219698/ZKSCAN4 (+328); chr6:28351122-28351182/ZSCAN12 (+16392), ZKSCAN3 (+33461); chr6:2836734
7-28367490/ZSCAN12 (+125); chr6:28411088-28411151/ZSCAN23 (+159); chr6:28414992-28415091/ZSCAN2
3 (−3763); chr6:28521795-28521836/GPX5 (+28027), SCAND3 (+33296); chr6:28557813-28557842/SCAND3
(−2716); chr6:28583970-28584268/SCAND3 (−29007), TRIM27 (+307649); chr6:28603170-28603274/SCAND3
(−48110), TRIM27 (+288546); chr6:28603662-28603720/SCAND3 (−48579), TRIM27 (+288077); chr6:28741335-2
8741425/SCAND3 (−186268), TRIM27 (+150388); chr6:28743595-28743613/SCAND3 (−188492), TRIM27 (+148
164); chr6:28743745-28743777/SCAND3 (−188649), TRIM27 (+148007); chr6:28747873-28747931/SCAND3
(−192790), TRIM27 (+143866); chr6:28945154-28945232/TRIM27 (−53425), ZNF311 (+27842); chr6:28945445-28
945479/TRIM27 (−53694), ZNF311 (+27573); chr6:28956512-28956659/TRIM27 (−64818), ZNF311 (+16449); ch
r6:29521012-29521075/MAS1L (−65365), UBD (+6658); chr6:29521536-29521687/MAS1L (−65933), UBD (+609
0); chr6:29691244-29691311/HLA-F (+161); chr6:29717135-29717187/LOC554223 (−42522), HLA-F (+26044);
chr6:29759980-29760109/LOC554223 (+362); chr6:29760264-29760328/LOC554223 (+613); chr6:29795870-2
9796067/HLA-A (−114278), HLA-G (+1213); chr6:29796190-29796238/HLA-A (−114033), HLA-G (+1458); chr6:2
9855705-29855774/HLA-A (−54507), HLA-G (+60984); chr6:29855913-29855969/HLA-A (−54306), HLA-G (+611
85); chr6:29894390-29894533/HLA-A (−15785), HLA-G (+99706); chr6:29894907-29894945/HLA-A (−15321), H
LA-G (+100170); chr6:29910993-29911035/HLA-A (+767); chr6:29911294-29911317/HLA-J (−62911), HLA-A
(+1059); chr6:29974344-29974511/HLA-J (+211); chr6:29974862-29974899/HLA-J (+664); chr6:30028942-30028
990/ZNRD1 (−70); chr6:30039201-30039277/PPP1R11 (+4307), RNF39 (+4389); chr6:30071174-30071231/RN
F39 (−27575), TRIM31 (+9664); chr6:30095581-30095705/TRIM31 (−14776), TRIM40 (−8867); chr6:30139870-3
0140135/TRIM10 (−11292), TRIM26 (+41268); chr6:30294904-30295069/TRIM39-RPP21 (−2101); chr6:304195
69-30419594/HLA-E (−37601), TRIM39-RPP21 (+122494); chr6:30420894-30421036/HLA-E (−36218), TRIM39-
RPP21 (+123877); chr6:30431788-30431824/HLA-E (−25377), TRIM39-RPP21 (+134718); chr6:30432081-3043
2151/HLA-E (−25067), TRIM39-RPP21 (+135028); chr6:30457970-30458007/HLA-E (+806); chr6:30538789-305
38835/ABCF1 (−358); chr6:30585007-30585030/MRPS18B (−467), PPP1R10 (+1); chr6:30653700-30653727/D
HX16 (−12884), PPP1R18 (+1379); chr6:30654509-30654538/PPP1R18 (+569); chr6:30655248-30655288/PPP
1R18 (−175); chr6:30689389-30689433/MDC1 (−3953), TUBB (+1254); chr6:30698783-30698904/TUBB (+1068
7), FLOT1 (+11609); chr6:30876071-30876136/GTF2H4 (+127); chr6:30882630-30882707/VARS2 (+561); chr6:
31125929-31125949/CCHCR1 (−373), TCF19 (−364); chr6:31239023-31239062/HLA-C (+870); chr6:31466100-
31466118/MICB (+254); chr6:31509858-31509890/DDX39B (+378); chr6:31525860-31525914/LTA (−13989), N
FKBIL1 (+10534); chr6:31783321-31783481/HSPA1L (−566), HSPA1A (+110); chr6:31795391-31795435/HSPA
1B (−99); chr6:31795740-31795753/HSPA1B (+235); chr6:31870063-31870083/EHMT2 (−4609), ZBTB12 (−304);
chr6:31939844-31939892/DOM3Z (+164), STK19 (+222); chr6:32055369-32055449/TNXB (+21742), CYP21A2
(+49316); chr6:32064371-32064488/TNXB (+12721), CYP21A2 (+58337); chr6:32064638-32064659/TNXB (+1
2502), CYP21A2 (+58556); chr6:32116779-32116857/PPT2 (−4411), PRRT1 (+2902); chr6:32116982-32117028/
PPT2 (−4224), PRRT1 (+2715); chr6:32632741-32632756/HLA-DQB1 (+1717), HLA-DQA1 (+22252); chr6:328
12363-32812458/PSMB8 (−595); chr6:32813440-32813581/PSMB8 (−1695); chr6:32862251-32862318/PSMB9
(+40347), HLA-DMB (+46562); chr6:33085259-33085325/HLA-DPB1 (+41589), COL11A2 (+74953); chr6:33239
847-33239880/VPS52 (−202), RPS18 (+12); chr6:33245914-33245989/B3GALT4 (+1035), WDR46 (+11352); ch
r6:33256964-33257017/PFDN6 (−387), WDR46 (+313); chr6:33267098-33267177/RGL2 (+27); chr6:33290887-
33290933/DAXX (−117); chr6:33679641-33679699/C6orf125 (−166); chr6:33714040-33714120/IP6K3 (+682); ch
r6:34203174-34203203/HMGA1 (−1461); chr6:34203686-34203750/HMGA1 (−932); chr6:34206133-34206174/H
MGA1 (+1504), C6orf1 (+10750); chr6:34360080-34360099/C6orf1 (−143186), RPS10-NUDT3 (+33812); chr6:3
4664072-34664096/C6orf106 (+541); chr6:34664350-34664389/C6orf106 (+255); chr6:35109013-35109085/TC
P11 (+138); chr6:35480045-35480091/TULP1 (+579); chr6:35699486-35699554/FKBP5 (−42801), ARMC12 (−5
339); chr6:35992248-35992355/MAPK14 (−3152), SLC26A8 (+3); chr6:36098533-36098619/MAPK13 (+314); ch
r6:36332706-36332853/ETV7 (+22797), PNPLA1 (+94543); chr6:36355989-36356014/ETV7 (−425); chr6:36391
327-36391390/ETV7 (−35782), KCTD20 (−19185); chr6:36628590-36628769/CDKN1A (−15557), SRSF3 (+6659
0); chr6:36737478-36737591/CPNE5 (+69685), CDKN1A (+93298); chr6:36930543-36930689/PI16 (+8407), M
TCH1 (+23333); chr6:36953611-36953670/MTCH1 (+308); chr6:36954245-36954286/MTCH1 (−317); chr6:3740
1307-37401333/FTSJD2 (+413); chr6:37467672-37467695/CCDC167 (+16); chr6:37504375-37504401/CCDC1
67 (−36688), MDGA1 (+161378); chr6:37616757-37616960/CCDC167 (−149159), MDGA1 (+48907); chr6:37664
459-37664481/CCDC167 (−196770), MDGA1 (+1296); chr6:37666844-37666866/MDGA1 (−1089); chr6:386828
43-38682906/DNAH8 (−242); chr6:38683141-38683211/DNAH8 (+59); chr6:39016325-39016390/GLP1R (−199);
chr6:39016630-39016678/GLP1R (+97); chr6:39016952-39017039/GLP1R (+439); chr6:39082382-39082452/S
AYSD1 (+448); chr6:40995826-40995898/LRFN2 (−440736), UNC5CL (+11076); chr6:41040200-41040300/NFY
A (−457), C6orf130 (−62); chr6:41336735-41336764/FOXP4 (−177414), NCR2 (+33222); chr6:41339559-413396
01/FOXP4 (−174584), NCR2 (+36052); chr6:41341604-41341760/FOXP4 (−172482), NCR2 (+38154); chr6:4147
1078-41471168/FOXP4 (−43041), NCR2 (+167595); chr6:41513312-41513366/FOXP4 (−825); chr6:41515584-4
1515777/MDFI (−90514), FOXP4 (+1517); chr6:41515897-41516279/MDFI (−90107), FOXP4 (+1924); chr6:4152
8306-41528355/MDFI (−77864), FOXP4 (+14167); chr6:41605344-41605439/MDFI (−803); chr6:41605952-4160
6037/MDFI (−200); chr6:41606357-41606527/MDFI (+247); chr6:41607089-41607176/MDFI (+938); chr6:41750
287-41750364/TOMM6 (−4174), FRS3 (−2696); chr6:41863546-41863606/USP49 (−477); chr6:42072051-42072
090/GUCA1A (−51073), TAF8 (+53820); chr6:42072402-42072484/GUCA1A (−50701), TAF8 (+54192); chr6:421
45905-42145975/GUCA1B (+16754), GUCA1A (+22796); chr6:42335129-42335230/MRPS10 (−149547), TRER
F1 (+84603); chr6:42397115-42397190/MRPS10 (−211520), TRERF1 (+22630); chr6:42531868-42531901/UBR
2 (+125); chr6:42694917-42694994/PRPH2 (−4598); chr6:42695143-42695198/PRPH2 (−4813); chr6:42879569-
42879621/PTCRA (−4132); chr6:43027554-43027577/MRPL2 (−324), KLC4 (−63); chr6:43043964-43044000/PT
K7 (−47); chr6:43211462-43211478/TTBK1 (+248); chr6:43244032-43244096/SLC22A7 (−21934), TTBK1 (+328
42); chr6:43252833-43252887/SLC22A7 (−13138), TTBK1 (+41638); chr6:43478020-43478058/YIPF3 (+6689),
TJAP1 (+32778); chr6:43612899-43613052/RSPH9 (+209); chr6:43855215-43855325/VEGFA (+117324), MRP
L14 (+239921); chr6:43970849-43970953/MRPL14 (+124290), VEGFA (+232955); chr6:44095055-44095109/T
MEM63B (−294), MRPL14 (+109); chr6:44187075-44187131/SLC29A1 (−4193); chr6:44205645-44205700/HSP9
0AB1 (−9176), SLC29A1 (+14377); chr6:44205836-44205876/HSP90AB1 (−8993), SLC29A1 (+14560); chr6:442
24923-44224958/SLC35B2 (+342); chr6:44244012-44244119/TMEM151B (+5586), AARS2 (+36997); chr6:443
10390-44310508/CDC5L (−44802), AARS2 (−29386); chr6:44528957-44529119/RUNX2 (−767016), CDC5L (+17
3787); chr6:45390316-45390346/SUPT3H (−44661), CLIC5 (+657754); chr6:45391881-45391990/SUPT3H (−46
266), CLIC5 (+656149); chr6:45631136-45631602/SUPT3H (−285699), CLIC5 (+416716); chr6:46097622-46097
694/ENPP4 (−43); chr6:47445441-47445477/CD2AP (−66); chr6:48036583-48036619/C6orf138 (−176); chr6:506
76232-50676330/TFAP2D (−4976); chr6:50682339-50682448/TFAP2B (−104045), TFAP2D (+1137); chr6:50682
684-50682711/TFAP2B (−103741), TFAP2D (+1441); chr6:50682941-50682991/TFAP2B (−103473), TFAP2D
(+1709); chr6:50692213-50692299/TFAP2B (−94183), TFAP2D (+10999); chr6:50787877-50787949/TFAP2B (+1
474); chr6:50791495-50791550/TFAP2B (+5084); chr6:50810714-50810936/TFAP2B (+24386); chr6:50818185-
50818348/TFAP2B (+31828); chr6:53409520-53409552/GCLC (+391); chr6:53660874-53660908/MLIP (−3153
54), LRRC1 (+1357); chr6:54711492-54711546/HCRTR2 (−327552), TINAG (+538316); chr6:55039231-550393
63/HCRTR2 (+226); chr6:56716300-56716389/DST (−208651), KIAA1586 (−195039); chr6:56954778-56954813/
ZNF451 (−32); chr6:58141836-58141898/PRIM2 (+959445); chr6:58142246-58142289/PRIM2 (+959846); chr6:
58147511-58147739/PRIM2 (+965203); chr6:58148434-58148644/PRIM2 (+966117); chr6:58149247-58149292/
PRIM2 (+966848); chr6:58180257-58180441/PRIM2 (+997927); chr6:62995875-62996077/KHDRBS2 (+124); c
hr6:70992049-70992136/COL9A1 (+20693), COL19A1 (+415645); chr6:71123704-71123746/C6orf57 (−152900),
COL9A1 (−110939); chr6:71666681-71666698/B3GAT2 (+98); chr6:72129850-72129926/RIMS1 (−466762), O
GFRL1 (+131411); chr6:72130045-72130190/RIMS1 (−466532), OGFRL1 (+131641); chr6:72130499-72130646/
RIMS1 (−466077), OGFRL1 (+132096); chr6:73331851-73331875/KCNQ5 (+292); chr6:73839714-73839781/K
HDC1 (+133159), KCNQ5 (+508177); chr6:74024483-74024515/KHDC1 (−51592), DPPA5 (+39500); chr6:7416
1192-74161232/MB21D1 (+831); chr6:74161499-74161684/MB21D1 (+451); chr6:74161798-74161830/MB21D
1 (+229); chr6:74405392-74405407/CD109 (−108); chr6:75911708-75911834/COL12A1 (+3852); chr6:7591470
5-75914738/COL12A1 (+901); chr6:75915121-75915170/COL12A1 (+477); chr6:75915384-75915426/COL12A
1 (+218); chr6:75917462-75917532/COL12A1 (−1874); chr6:75953516-75953638/COX7A2 (+67); chr6:7599377
3-75993870/TMEM30A (+810); chr6:78172276-78172322/HTR1B (+821); chr6:78173726-78173771/HTR1B (−6
29); chr6:78359940-78360072/HTR1B (−186886); chr6:79620877-79620907/IRAK1BP1 (+43703), PHIP (+1671
19); chr6:79786979-79787024/PHIP (+1009), IRAK1BP1 (+209813); chr6:79944328-79944379/HMGN3 (+101);
chr6:80246851-80246889/LCA5 (+277); chr6:80341024-80341054/SH3BGRL2 (+39); chr6:82461689-82461728/
IBTK (+495739); chr6:84417701-84417908/SNAP91 (+1042), PRSS35 (+195611); chr6:84418064-84418260/S
NAP91 (+685); chr6:84418888-84418915/SNAP91 (−55); chr6:84563285-84563416/RIPPLY2 (+366); chr6:8548
3167-85483210/NT5E (−676113), TBX18 (−9290); chr6:85483524-85483759/NT5E (−675660), TBX18 (−9743); c
hr6:85484111-85484298/NT5E (−675097), TBX18 (−10306); chr6:85484626-85484716/NT5E (−674631), TBX18
(−10772); chr6:86352947-86352992/SYNCRIP (+73); chr6:87647309-87647316/HTR1E (+289); chr6:87865161-
87865215/ZNF292 (−81); chr6:88182368-88182441/SLC35A1 (−238); chr6:88875568-88875621/CNR1 (+172); c
hr6:88876238-88876284/CNR1 (−494); chr6:88876549-88876572/CNR1 (−794); chr6:88877422-88877474/CNR
1 (−1681); chr6:89673535-89673565/RNGTT (−202); chr6:89827935-89827990/SRSF12 (−163); chr6:91005179-
91005208/BACH2 (+1433), GJA10 (+401006); chr6:91005631-91005660/BACH2 (+981); chr6:91006753-91006
779/BACH2 (−139); chr6:94126291-94126366/EPHA7 (+2971); chr6:94126613-94126642/EPHA7 (+2672); chr6:
94127066-94127085/EPHA7 (+2224); chr6:97284935-97284998/GPR63 (+386); chr6:99281037-99281067/PO
U3F2 (−1528); chr6:99290473-99290709/POU3F2 (+8011), FBXL4 (+105258); chr6:99291342-99291586/POU3
F2 (+8884), FBXL4 (+104385); chr6:99291697-99291729/POU3F2 (+9133), FBXL4 (+104136); chr6:99295864-
99296061/POU3F2 (+13383), FBXL4 (+99886); chr6:99296365-99296407/POU3F2 (+13806), FBXL4 (+99463);
chr6:100038707-100038785/CCNC (−22056), PRDM13 (−15904); chr6:100039154-100039274/CCNC (−22524),
PRDM13 (−15436); chr6:100050462-100050505/PRDM13 (−4166); chr6:100050811-100050858/PRDM13 (−3815);
chr6:100051112-100051359/PRDM13 (−3414); chr6:100051508-100051771/PRDM13 (−3010); chr6:10005523
2-100055269/PRDM13 (+601); chr6:100067019-100067144/PRDM13 (+12432), MCHR2 (+375032); chr6:10044
1308-100441497/MCHR2 (+711); chr6:100441738-100441761/MCHR2 (+364); chr6:100895637-100895834/MC
HR2 (−453622), SIM1 (+16075); chr6:100897261-100897520/MCHR2 (−455277), SIM1 (+14420); chr6:1009016
17-100901711/MCHR2 (−459550), SIM1 (+10147); chr6:100903405-100903560/MCHR2 (−461369), SIM1 (+832
8); chr6:100903781-100903832/MCHR2 (−461693), SIM1 (+8004); chr6:100906167-100906245/MCHR2 (−4640
92), SIM1 (+5605); chr6:100912446-100912465/SIM1 (−645); chr6:100913051-100913266/SIM1 (−1348); chr6:1
00917310-100917457/SIM1 (−5573), ASCC3 (+411840); chr6:101846790-101846865/GRIK2 (−33); chr6:101850
629-101850725/GRIK2 (+3816); chr6:105388321-105388399/HACE1 (−80566), LIN28B (−16563); chr6:1053887
17-105389493/HACE1 (−81311), LIN28B (−15818); chr6:105584320-105584366/BVES (+200); chr6:106429476-
106429589/PREP (−578534), PRDM1 (−104662); chr6:106434068-106434152/PREP (−583111), PRDM1 (−1000
85); chr6:107436198-107436232/C6orf203 (+86808), PDSS2 (+344564); chr6:107955552-107955819/SOBP
(+144369), SCML4 (+189835); chr6:107955952-107956396/SOBP (+144857), SCML4 (+189347); chr6:10843815
7-108438260/NR2E1 (−49006), OSTM1 (−42268); chr6:108440645-108440744/NR2E1 (−46520), OSTM1 (−4475
4); chr6:108487006-108487077/NR2E1 (−173); chr6:108488598-108488821/NR2E1 (+1495), SNX3 (+93754); c
hr6:108489279-108489661/NR2E1 (+2255), SNX3 (+92994); chr6:108489976-108490034/NR2E1 (+2790), SN
X3 (+92459); chr6:108490246-108490296/NR2E1 (+3056), SNX3 (+92193); chr6:108490515-108490537/NR2E
1 (+3311), SNX3 (+91938); chr6:108491001-108491107/NR2E1 (+3839), SNX3 (+91410); chr6:108495818-108
495915/NR2E1 (+8652), SNX3 (+86597); chr6:108497797-108497826/NR2E1 (+10597), SNX3 (+84652); chr6:
108880358-108880410/FOXO3 (−1685); chr6:108880766-108880789/FOXO3 (−1291); chr6:108882931-108882
975/FOXO3 (+884); chr6:109169765-109169810/ARMC2 (+169); chr6:109331158-109331216/SESN1 (+84521),
ARMC2 (+161568); chr6:109762478-109762527/PPIL6 (−129), SMPD2 (+572); chr6:110012408-110012443/A
KD1 (−11), FIG4 (+2); chr6:110299445-110299484/GPR6 (−833); chr6:110300307-110300364/GPR6 (+38); chr6:
110300542-110300584/GPR6 (+265); chr6:110797874-110797926/SLC22A16 (−56); chr6:111136480-1111365
93/CDK19 (−125); chr6:112364428-112364491/FYN (−323195), WISP3 (−10911); chr6:114291956-114292009/H
DAC2 (+376); chr6:116783104-116783139/FAM26F (+566); chr6:116989950-116989984/ZUFSP (+6); chr6:117
584608-117584675/VGLL2 (−2079); chr6:117586644-117586846/VGLL2 (+24); chr6:117591597-117591683/VG
LL2 (+4919), ROS1 (+155378); chr6:117804434-117804487/DCBLD1 (+641); chr6:117997192-117997256/NUS
1 (+607); chr6:118228232-118228323/SLC35F1 (−411); chr6:118229390-118229416/SLC35F1 (+714); chr6:118
229573-118229616/SLC35F1 (+906); chr6:118241309-118241394/PLN (−628090), SLC35F1 (+12663); chr6:12
1655628-121655675/C6orf170 (−8); chr6:123111136-123111186/CLVS2 (−206421), SMPDL3A (+1190); chr6:12
3317007-123317072/CLVS2 (−542); chr6:125283490-125283600/RNF217 (−20969); chr6:126068022-12606806
8/HEY2 (−2687); chr6:127439273-127439321 /RSPO3 (−751); chr6:127440963-127441030/RSPO3 (+949); chr6:
127442071-127442089/RNF146 (−145747), RSPO3 (+2032); chr6:127839821-127839929/C6orf174 (+625); chr
6:129204304-129204360/LAMA2 (+46); chr6:130341153-130341164/TMEM200A (−417103), L3MBTL3 (+1431);
chr6:130341270-130341286/TMEM200A (−416984), L3MBTL3 (+1550); chr6:130687193-130687224/TMEM200
A (−71053), L3MBTL3 (+347481); chr6:131384220-131384256/EPB41L2 (+224); chr6:132722142-132722157/M
OXD1 (+514); chr6:133562268-133562348/EYA4 (−187); chr6:133563095-133563157/EYA4 (+631); chr6:13415
9302-134159347/TCF21 (−50934), EYA4 (+596830); chr6:134176313-134176376/TCF21 (−33914), EYA4 (+613
850); chr6:134211019-134211111/TCF21 (+806); chr6:134213988-134214076/TBPL1 (−59276), TCF21 (+3773);
chr6:134495559-134495595/SLC2A12 (−121788), SGK1 (+143619); chr6:135376414-135376460/HBS1L (−401);
chr6:137244149-137244235/SLC35D3 (+790); chr6:137244466-137245086/SLC35D3 (+1374), IL20RA (+121
522); chr6:137808894-137809000/IFNGR1 (−268380), OLIG3 (+6584); chr6:137809366-137809445/IFNGR1 (−2
68839), OLIG3 (+6125); chr6:137809980-137810032/IFNGR1 (−269439), OLIG3 (+5525); chr6:137814619-1378
14653/OLIG3 (+895); chr6:137815171-137815224/OLIG3 (+333); chr6:137818599-137818618/OLIG3 (−3078); c
hr6:138482869-138482902/KIAA1244 (−167); chr6:139094495-139094532/ECT2L (−22734), HEBP2 (+369178);
chr6:142409702-142409750/NMBR (+210); chr6:143857701-143857758/PHACTR2 (−141372), FUCA2 (−24710);
chr6:144416024-144416102/SF3B5 (+691); chr6:144471890-144471924/STX11 (+253); chr6:146350409-1463
50452/RAB32 (−514397), GRM1 (+1649); chr6:146350600-146350619/RAB32 (−514218), GRM1 (+1828); chr6:
146350721-146350762/RAB32 (−514086), GRM1 (+1960); chr6:147828394-147828454/STXBP5 (+302930); chr
6:149771896-149771913/ZC3H12D (+34243), TAB2 (+132469); chr6:149772273-149772292/ZC3H12D (+3386
5), TAB2 (+132847); chr6:149803615-149803686/ZC3H12D (+2497), TAB2 (+164215); chr6:150038699-15003
8781/LATS1 (+652); chr6:150067633-150067677/PCMT1 (−3176), NUP43 (+33); chr6:150070625-150070660/N
UP43 (−2955), PCMT1 (−188); chr6:150247166-150247248/RAET1G (−2993); chr6:150262374-150262428/ULB
P2 (−735); chr6:150275488-150275554/ULBP1 (−9622), ULBP2 (+12385); chr6:150276087-150276322/ULBP1
(−8938), ULBP2 (+13069); chr6:150285369-150285544/ULBP1 (+314); chr6:150285886-150286099/ULBP1 (+85
0); chr6:150335570-150335739/RAET1L (+11013), ULBP1 (+50512); chr6:150336182-150336239/RAET1L (+1
0457), ULBP1 (+51068); chr6:150359193-150359438/RAET1L (−12648), ULBP3 (+30967); chr6:150389779-150
389820/ULBP3 (+483); chr6:151186912-151187024/MTHFD1L (+153); chr6:151561341-151561368/AKAP12
(+221); chr6:151561992-151562056/AKAP12 (+890); chr6:152002877-152002939/RMND1 (−229592), ESR1 (−125
546); chr6:152128917-152128956/ESR1 (+483); chr6:152129109-152129155/ESR1 (+678); chr6:152129604-15
2129680/ESR1 (+1188), SYNE1 (+828892); chr6:152957781-152957815/SYNE1 (+736); chr6:153452130-1534
52143/RGS17 (+252); chr6:153452755-153452788/RGS17 (−383); chr6:155316221-155316256/TIAM2 (−95184),
SCAF8 (+261727); chr6:157342583-157342629/ARID1B (+243542), TMEM242 (+402647); chr6:157342960-1
57342977/ARID1B (+243905), TMEM242 (+402284); chr6:157557297-157557711/TMEM242 (+187749), ARID1
B (+458440); chr6:157557827-157557983/TMEM242 (+187348), ARID1B (+458841); chr6:158589110-1585891
38/GTF2H5 (−255), SERAC1 (+188); chr6:158937461-158937510/TMEM181 (−19982), TULP4 (+203794); chr6:
159065777-159065792/DYNLT1 (+19); chr6:159240349-159240380/EZR (−1025); chr6:159360785-159360911/
EZR (−121508), TAGAP (+105336); chr6:159519932-159520019/FNDC1 (−70453), TAGAP (−53792); chr6:1595
90087-159590154/FNDC1 (−308); chr6:159590762-159590971/FNDC1 (+438); chr6:159595461-159595527/FN
DC1 (+5065), SOD2 (+518859); chr6:159654541-159654578/FNDC1 (+64131), SOD2 (+459793); chr6:160390
074-160390092/IGF2R (−48); chr6:160769795-160769827/SLC22A3 (+386); chr6:161100418-161100580/PLG
(−22726), LPA (−13092); chr6:161188084-161188108/MAP3K4 (−224726), PLG (+64871); chr6:161188275-16118
8410/MAP3K4 (−224479), PLG (+65118); chr6:161352146-161352198/MAP3K4 (−60650), PLG (+228947); chr6:
161352308-161352366/MAP3K4 (−60485), PLG (+229112); chr6:163148885-163149077/PARK2 (−147), PACR
G (+817); chr6:163670668-163670721/PARK2 (−521861), QKI (−164980); chr6:163756920-163756951/PARK2
(−608102), QKI (−78739); chr6:163834383-163834405/QKI (−1281); chr6:163834533-163834710/QKI (−1053); chr
6:163834902-163834987/QKI (−730); chr6:164051832-164051877/QKI (+216180); chr6:166073947-166073993/
PDE10A (+1618); chr6:166075125-166075258/PDE10A (+396); chr6:166077633-166077668/PDE10A (−2063);
chr6:166233105-166233192/PDE10A (−157561), T (+348982); chr6:166263716-166263789/PDE10A (−188165),
T (+318378); chr6:166267892-166268182/PDE10A (−192449), T (+314094); chr6:166402651-166402752/PDE1
0A (−327114), T (+179429); chr6:166582827-166582941/T (−753); chr6:166583096-166583152/T (−993); chr6:16
6666860-166666879/T (−84739), PRR18 (+55001); chr6:166667126-166667161/T (−85013), PRR18 (+54727); c
hr6:166755149-166755191/SFT2D1 (+821); chr6:166755841-166755866/SFT2D1 (+137); chr6:166756074-166
756095/SFT2D1 (−94); chr6:167369494-167369552/RNASET2 (+554); chr6:167411904-167411948/FGFR1OP
(−890); chr6:168079131-168079180/TCP10 (−281158), MLLT4 (−148515); chr6:168107202-168107248/TCP10
(−309227), MLLT4 (−120446); chr6:168197262-168197330/TCP10 (−399298), MLLT4 (−30375); chr6:168479586-1
68479630/FRMD1 (+231); chr6:168501905-168502095/FRMD1 (−22161), DACT2 (+218402); chr6:168502314-1
68502653/FRMD1 (−22645), DACT2 (+217918); chr6:168719803-168719889/DACT2 (+556); chr6:168785421-1
68785529/DACT2 (−65073), SMOC2 (−56356); chr6:168841479-168841 502/SMOC2 (−340); chr6:168843061-16
8843080/SMOC2 (+1240), THBS2 (+811066); chr6:169572678-169572759/THBS2 (+81418), SMOC2 (+730888);
chr6:169664497-169664529/C6orf120 (−437744), THBS2 (−10376); chr6:170338560-170338747/C6orf70 (+18
6933), DLL1 (+261043); chr6:170403191-170403263/DLL1 (+196470), C6orf70 (+251506); chr6:170467266-17
0467332/DLL1 (+132398), C6orf70 (+315578); chr6:170468270-170468382/DLL1 (+131371), C6orf70 (+316605);
chr6:170581038-170581095/DLL1 (+18630), C6orf70 (+429346); chr6:170598352-170598458/DLL1 (+1292),
C6orf70 (+446684); chr6:170605876-170605909/FAM120B (−9951), DLL1 (−6196); chr6:170615414-170615448/
FAM120B (−413); chr7:63841-63883/FAM20C (−129107); chr7:150020-150083/FAM20C (−42917); chr7:164217-
164302/FAM20C (−28709); chr7:329231-329271/FAM20C (+136282), PDGFA (+230230); chr7:329694-329764/
FAM20C (+136760), PDGFA (+229752); chr7:330006-330425/FAM20C (+137247), PDGFA (+229265); chr7:33
0678-330720/FAM20C (+137730), PDGFA (+228782); chr7:330828-331176/FAM20C (+138033), PDGFA (+228
479); chr7:391156-391393/PDGFA (+168206), FAM20C (+198306); chr7:391842-391887/PDGFA (+167616), F
AM20C (+198896); chr7:400554-400586/PDGFA (+158911), FAM20C (+207601); chr7:416004-416065/PDGFA
(+143446), FAM20C (+223066); chr7:492224-492270/PDGFA (+67234), FAM20C (+299278); chr7:557660-557
697/PDGFA (+1802), FAM20C (+364710); chr7:557991-558076/PDGFA (+1447), FAM20C (+365065); chr7:560
689-560731/PDGFA (−1229); chr7:630491-630560/HEATR2 (−135812), PDGFA (−71045); chr7:633046-633068/
HEATR2 (−133281), PDGFA (−73576); chr7:641090-641359/HEATR2 (−125113), PDGFA (−81744); chr7:644133-
644331/HEATR2 (−122106), PDGFA (−84751); chr7:752306-752350/PDGFA (−192847), HEATR2 (−14010); chr7:
766893-766929/PRKAR1B (+402), HEATR2 (+573); chr7:852941-853018/PRKAR1B (−85667), SUN1 (−19162);
chr7:948458-948675/GET4 (+32376), ADAP1 (+45739); chr7:949063-949138/GET4 (+32910), ADAP1 (+45205);
chr7:961746-961796/ADAP1 (+32535), GET4 (+45580); chr7:1014866-1014936/COX19 (+334); chr7:1036323-
1036365/GPR146 (−60797), CYP2W1 (+13509); chr7:1051889-1051992/GPR146 (−45200), CYP2W1 (+29106);
chr7:1067812-1067869/GPR146 (−29300), CYP2W1 (+45006); chr7:1068113-1068265/GPR146 (−28952), CYP
2W1 (+45354); chr7:1263580-1263658/ZFAND2A (−63764), UNCX (−9035); chr7:1269464-1269552/UNCX (−314
6); chr7:1273388-1273589/UNCX (+835); chr7:1274925-1275045/UNCX (+2331), MICALL2 (+224124); chr7:12
75196-1275257/UNCX (+2573), MICALL2 (+223882); chr7:1275682-1275733/UNCX (+3054), MICALL2 (+2234
01); chr7:1275917-1276291/UNCX (+3450), MICALL2 (+223005); chr7:1286111-1286444/UNCX (+13624), MIC
ALL2 (+212831); chr7:1286546-1286612/UNCX (+13925), MICALL2 (+212530); chr7:1286754-1286886/UNCX
(+14166), MICALL2 (+212289); chr7:1287371-1287431/UNCX (+14747), MICALL2 (+211708); chr7:1297126-12
97153/UNCX (+24486), MICALL2 (+201969); chr7:1362844-1362890/UNCX (+90213), MICALL2 (+136242); chr
7:1408771-1408874/MICALL2 (+90286), UNCX (+136169); chr7:1409101-1409305/MICALL2 (+89906), UNCX
(+136549); chr7:1443685-1443733/MICALL2 (+55400), UNCX (+171055); chr7:1550489-1550510/MAFK (−1986
8), INTS1 (−6482); chr7:1552820-1552940/MAFK (−17488), INTS1 (−8862); chr7:1570870-1570892/MAFK (+513);
chr7:1610195-1610219/ELFN1 (−138591), TMEM184A (−14141); chr7:1703872-1704118/TMEM184A (−10792
9), ELFN1 (−44803); chr7:1707619-1707646/TMEM184A (−111567), ELFN1 (−41165); chr7:1709562-1709617/T
MEM184A (−113524), ELFN1 (−39208); chr7:1709923-1709955/TMEM184A (−113873), ELFN1 (−38859); chr7:17
10244-1710324/TMEM184A (−114218), ELFN1 (−38514); chr7:1858976-1859004/ELFN1 (+110192), MAD1L1
(+413593); chr7:1888023-1888100/ELFN1 (+139264), MAD1L1 (+384521); chr7:1959793-1959955/ELFN1 (+211
076), MAD1L1 (+312709); chr7:2059345-2059377/MAD1L1 (+213222), ELFN1 (+310563); chr7:2059920-20599
67/MAD1L1 (+212639), ELFN1 (+311146); chr7:2272092-2272137/MAD1L1 (+468); chr7:2289037-2289150/NU
DT1 (+7237), SNX8 (+65005); chr7:2394123-2394183/EIF3B (−321); chr7:2492062-2492106/LFNG (−67395), C
HST12 (+48889); chr7:2511056-2511137/LFNG (−48382), CHST12 (+67902); chr7:2548136-2548205/LFNG (−1
1308), CHST12 (+104976); chr7:2550548-2550612/LFNG (−8899), CHST12 (+107385); chr7:2568431-2568575/
LFNG (+9024), BRAT1 (+26889); chr7:2727815-2727853/AMZ1 (+8671), GNA12 (+156125); chr7:2749637-274
9692/AMZ1 (+30502), GNA12 (+134294); chr7:2753167-2753291/AMZ1 (+34066), GNA12 (+130730); chr7:275
7284-2757339/AMZ1 (+38149), GNA12 (+126647); chr7:2757631-2757663/AMZ1 (+38484), GNA12 (+126312);
chr7:3083288-3083330/CARD11 (+200); chr7:3134519-3134538/SDK1 (−206551), CARD11 (−51020); chr7:334
1409-3341569/SDK1 (+409); chr7:4304924-4304952/FOXK1 (−416992), SDK1 (+963858); chr7:4653441-46535
19/FOXK1 (−68450); chr7:4765311-4765371/KIAA0415 (−49921), FOXK1 (+43411); chr7:4831857-4831895/KIA
A0415 (+16614), PAPOLB (+69749); chr7:4848821-4848977/KIAA0415 (+33637), PAPOLB (+52726); chr7:488
5495-4885531/PAPOLB (+16112), KIAA0415 (+70251); chr7:4923043-4923229/RADIL (+199); chr7:4998587-4
998770/MMD2 (+165); chr7:5013858-5013879/RBAK (−71684), MMD2 (−15025); chr7:5014367-5014425/RBAK
(−71157), MMD2 (−15552); chr7:5085484-5085523/RBAK (−49); chr7:5335676-5335706/SLC29A4 (+13130), TN
RC18 (+127486); chr7:5497509-5497564/TNRC18 (−34360), FBXL18 (+55862); chr7:5526590-5526634/TNRC1
8 (−63435), FBXL18 (+26787); chr7:5633919-5633968/FSCN1 (+1508), RNF216 (+187417); chr7:5938538-5938
571/CCZ1 (+214); chr7:6121016-6121080/USP42 (−23502), EIF2AK1 (−22188); chr7:6121201-6121229/USP42
(−23335), EIF2AK1 (−22355); chr7:6487341-6487366/DAGLB (+289); chr7:6566236-6566246/KDELR2 (−42392),
GRID2IP (+24826); chr7:6570863-6570900/KDELR2 (−47033), GRID2IP (+20185); chr7:6573861-6573988/KDE
LR2 (−50076), GRID2IP (+17142); chr7:6576528-6576557/KDELR2 (−52694), GRID2IP (+14524); chr7:6703870-
6703915/ZNF12 (+42673), ZNF853 (+48366); chr7:8300809-8300874/ICA1 (+840); chr7:8301371-8301429/IC
A1 (+282); chr7:8301544-8301570/ICA1 (+125); chr7:8473456-8473479/NXPH1 (−117); chr7:8474242-8474515/
NXPH1 (+794); chr7:8475440-8475556/NXPH1 (+1913); chr7:8482298-8482669/NXPH1 (+8899); chr7:8482825-
8482884/NXPH1 (+9270); chr7:12151473-12151523/THSD7A (−279674), TMEM106B (−99350); chr7:12610665-
12610726/SCIN (+493); chr7:16793342-16793380/TSPAN13 (+10); chr7:18126062-18126082/HDAC9 (−40981
3), PRPS1L1 (−58586); chr7:18126458-18126465/HDAC9 (−409423), PRPS1L1 (−58976); chr7:19145894-19146
031/TWIST1 (+11332), HDAC9 (+610078); chr7:19146185-19146237/TWIST1 (+11084), HDAC9 (+610326); chr
7:19151883-19152223/TWIST1 (+5242), HDAC9 (+616168); chr7:19156062-19156125/TWIST1 (+1201), HDAC
9 (+620209); chr7:19156247-19156281/TWIST1 (+1031), HDAC9 (+620379); chr7:19156841-19156968/TWIST
1 (+390); chr7:19157567-19157633/TWIST1 (−305); chr7:19184426-19184564/FERD3L (+549); chr7:19184885-
19184960/FERD3L (+121); chr7:19186033-19186067/FERD3L (−1006); chr7:20817957-20817982/SP8 (+8538),
ABCB5 (+162725); chr7:20818619-20818669/SP8 (+7864), ABCB5 (+163399); chr7:20823331-20823382/SP8
(+3151), ABCB5 (+168112); chr7:20823879-20823919/SP8 (+2609), ABCB5 (+168654); chr7:20824144-208244
75/SP8 (+2198), ABCB5 (+169065); chr7:20824820-20824835/SP8 (+1680), ABCB5 (+169583); chr7:20837627-
20837650/SP4 (−630050), SP8 (−11131); chr7:21582641-21582791/DNAH11 (−117); chr7:21583278-21583303/
DNAH11 (+458); chr7:23053793-23053819/FAM126A (−36); chr7:23221437-23221489/NUPL2 (+17); chr7:2324
5819-23245836/GPNMB (−40488), NUPL2 (+24382); chr7:23245947-23245963/GPNMB (−40361), NUPL2 (+24
509); chr7:23287351-23287400/C7orf30 (−51564), GPNMB (+1060); chr7:23338826-23338950/C7orf30 (−52); c
hr7:24323329-24323343/NPY (−471); chr7:24324028-24324063/NPY (+239); chr7:24324185-24324248/NPY
(+410); chr7:24324689-24324754/NPY (+915); chr7:24324965-24325060/MPP6 (−288072), NPY (+1206); chr7:24
797037-24797067/DFNA5 (+31); chr7:25021176-25021241/OSBPL3 (−1449); chr7:25892504-25892540/NPVF
(−624417), NFE2L3 (−299325); chr7:25898205-25898445/NPVF (−630220), NFE2L3 (−293522); chr7:25898622-2
5898707/NPVF (−630560), NFE2L3 (−293182); chr7:25900377-25900433/NPVF (−632300), NFE2L3 (−291442);
chr7:26192647-26192666/NFE2L3 (+810); chr7:26897145-26897198/SKAP2 (+7169), SNX10 (+565657); chr7:
26897664-26897761/SKAP2 (+6628), SNX10 (+566198); chr7:27135771-27135979/HOXA1 (−250); chr7:27141
786-27141917/HOXA2 (+542); chr7:27143181-27143237/HOXA2 (−815); chr7:27187070-27187106/HOXA5 (−38
01), HOXA6 (+305); chr7:27187251-27187319/HOXA5 (−3998), HOXA6 (+108); chr7:27192675-27192758/HOX
A6 (−5324), HOXA7 (+3579); chr7:27195741-27195866/HOXA7 (+492); chr7:27196031-27196152/HOXA7 (+20
4); chr7:27199602-27199725/HOXA7 (−3368); chr7:27204004-27204090/HOXA7 (−7751), HOXA9 (+1102); chr7:
27204770-27205088/HOXA9 (+220); chr7:27205193-27205265/HOXA9 (−80); chr7:27205898-27206077/HOXA
9 (−839); chr7:27208953-27209044/HOXA9 (−3850); chr7:27209789-27209886/HOXA9 (−4689); chr7:27214262-
27214329/HOXA10 (−341); chr7:27223193-27223221/HOXA10 (−9252), HOXA11 (+1628); chr7:27225117-2722
5238/HOXA11 (−343); chr7:27225484-27225581/HOXA11 (−698); chr7:27227629-27227757/HOXA11 (−2858); c
hr7:27229018-27229103/HOXA11 (−4226); chr7:27239173-27239225/HOXA13 (+526); chr7:27244611-2724479
7/HOXA13 (−4979); chr7:27252819-27252895/EVX1 (−29307), HOXA13 (−13132); chr7:27260101-27260412/EV
X1 (−21907), HOXA13 (−20532); chr7:27275414-27275466/HOXA13 (−35715), EVX1 (−6724); chr7:27278994-27
279236/EVX1 (−3049); chr7:27279369-27279453/EVX1 (−2753); chr7:27284670-27284967/EVX1 (+2655), HIBA
DH (+417801); chr7:27285519-27285620/EVX1 (+3406), HIBADH (+417050); chr7:27285914-27285979/EVX1
(+3783), HIBADH (+416673); chr7:27286099-27286170/EVX1 (+3971), HIBADH (+416485); chr7:27289101-272
89169/EVX1 (+6971), HIBADH (+413485); chr7:27291119-27291238/EVX1 (+9015), HIBADH (+411441); chr7:2
8449291-28449658/CREB5 (−2669); chr7:28449782-28449857/CREB5 (−2324); chr7:28997646-28997676/TRIL
(+368); chr7:29233971-29234008/CHN2 (−131); chr7:29605950-29606055/WIPF3 (−240167), PRR15 (+2576); c
hr7:30174168-30174230/ZNRF2 (−149724), PLEKHA8 (+106222); chr7:30175446-30175572/ZNRF2 (−148414),
PLEKHA8 (+107532); chr7:30188691-30188777/ZNRF2 (−135189), PLEKHA8 (+120757); chr7:30634615-3063
4649/GARS (+451); chr7:30721943-30722018/CRHR2 (+17738), GARS (+87800); chr7:31092167-31092241/A
DCYAP1R1 (+128); chr7:31092699-31092713/ADCYAP1R1 (+630); chr7:31092880-31092894/ADCYAP1R1
(+811); chr7:31121821-31121918/ADCYAP1R1 (+29794), NEUROD6 (+258668); chr7:31375622-31375676/NEU
ROD6 (+4889), ADCYAP1R1 (+283573); chr7:31376358-31376385/NEUROD6 (+4166), ADCYAP1R1 (+28429
6); chr7:32110114-32110180/PDE1C (+228236), PPP1R17 (+383516); chr7:32110692-32110703/PDE1C (+227
685), PPP1R17 (+384067); chr7:32337983-32338043/PDE1C (+370); chr7:32338218-32338283/PDE1C (+132);
chr7:32467656-32467946/PDE1C (−129418), LSM5 (+62222); chr7:33101708-33101765/NT5C3 (+672); chr7:3
4360959-34361051/NPSR1 (−336892), BMPER (+416482); chr7:35225834-35226044/DPY19L2P1 (−165); chr7:
35226523-35226556/DPY19L2P1 (−766); chr7:35293087-35293182/TBX20 (+576); chr7:35293501-35293542/T
BX20 (+189); chr7:35293655-35293749/TBX20 (+9); chr7:35293978-35294031/TBX20 (−294); chr7:35295106-3
5295197/TBX20 (−1441); chr7:35297005-35297137/TBX20 (−3360); chr7:35297354-35297470/TBX20 (−3701); c
hr7:35298017-35298121/TBX20 (−4358); chr7:35298347-35298409/TBX20 (−4667); chr7:35298608-35298826/T
BX20 (−5006), HERPUD2 (+436055); chr7:35301009-35301101/TBX20 (−7344), HERPUD2 (+433717); chr7:354
94333-35494469/TBX20 (−200690), HERPUD2 (+240371); chr7:37392952-37393044/AOAH (−628844), ELMO1
(+95557); chr7:37487543-37487609/ELMO1 (+979); chr7:37487966-37487984/ELMO1 (+580); chr7:37960537-
37960562/SFRP4 (−4025), EPDR1 (+387); chr7:38670701-38670956/AMPH (+338); chr7:39649308-39649346/
RALA (−13825), POU6F2 (+631718); chr7:40174829-40174877/C7orf11 (−602), C7orf10 (+278); chr7:42159516-
42159575/INHBA (−416840), GLI3 (+117072); chr7:42267712-42267821/INHBA (−525061), GLI3 (+8851); chr7:
42533081-42533256/GLI3 (−256551), C7orf25 (+418982); chr7:42971730-42971804/MRPL32 (−172), PSMA2
(+38); chr7:43152208-43152413/HECW1 (+113); chr7:43153200-43153229/STK17A (−469477), HECW1 (+1017);
chr7:43945627-43945685/URGCP (+575); chr7:44114009-44114084/PGAM2 (−8861), POLM (+8082); chr7:441
43942-44144029/AEBP1 (+26); chr7:44163151-44163168/POLD2 (−13); chr7:44349607-44349655/CAMK2B
(+15599), YKT6 (+109053); chr7:44364679-44364698/CAMK2B (+541); chr7:44365082-44365117/CAMK2B (+13
0); chr7:44622046-44622092/TMED4 (−242); chr7:45299615-45299683/ADCY1 (−314476), RAMP3 (+102282); c
hr7:45613814-45613857/ADCY1 (−289); chr7:45690791-45690902/IGFBP1 (−237112), ADCY1 (+76722); chr7:4
5808106-45808164/IGFBP1 (−119824), ADCY1 (+194010); chr7:47576672-47576721/TNS3 (+45045); chr7:475
76886-47576916/TNS3 (+44841); chr7:47608342-47608494/TNS3 (+13324); chr7:47611206-47611274/TNS3
(+10502); chr7:47620648-47620718/TNS3 (+1059); chr7:48128299-48128376/UPP1 (−17); chr7:49813018-4981
3040/VWC2 (−228); chr7:49813192-49813267/VWC2 (−27); chr7:49813436-49813600/VWC2 (+261); chr7:4981
4093-49814121/VWC2 (+850); chr7:49814780-49814821/VWC2 (+1544), ZPBP (+318059); chr7:49815020-498
15116/VWC2 (+1811), ZPBP (+317792); chr7:49815364-49815656/VWC2 (+2253), ZPBP (+317350); chr7:5013
2777-50132801/C7orf72 (−2893), ZPBP (+71); chr7:50424630-50424694/IKZF1 (+80284), FIGNL1 (+93426); ch
r7:50861103-50861127/GRB10 (−61050), COBL (+523400); chr7:53287027-53287109/NONE; chr7:54609952-5
4609991/VSTM2A (−47); chr7:55087947-55087982/LANCL2 (−345176), EGFR (+1240); chr7:55517000-555170
36/LANCL2 (+83877), VOPP1 (+123182); chr7:55639355-55639426/VOPP1 (+809); chr7:56183702-56183846/
CHCHD2 (−9587); chr7:56242832-56242852/CHCHD2 (−68655), ZNF479 (+964729); chr7:56242963-56243208/
CHCHD2 (−68899), ZNF479 (+964485); chr7:56297549-56297602/CHCHD2 (−123389), ZNF479 (+909995); chr
7:56355487-56355682/CHCHD2 (−181398), ZNF479 (+851986); chr7:57063797-57063841/CHCHD2 (−889632),
ZNF479 (+143752); chr7:63250196-63250207/LOC643955 (−485768), ZNF727 (−255619); chr7:63250423-6325
0456/LOC643955 (−486006), ZNF727 (−255381); chr7:63361477-63361535/LOC643955 (−597072), ZNF727 (−1
44315); chr7:63560611-63560736/ZNF735 (−106907), ZNF727 (+54853); chr7:63642490-63642553/ZNF735 (−2
5059), ZNF727 (+136701); chr7:64029965-64030217/ZNF107 (−96420), ZNF680 (−6586); chr7:64030592-64030
697/ZNF107 (−95866), ZNF680 (−7140); chr7:64348968-64349041/ZNF273 (−14615), ZNF138 (+94234); chr7:64
349882-64349983/ZNF273 (−13687), ZNF138 (+95162); chr7:64407672-64407780/ZNF117 (+43688), ZNF273
(+44106); chr7:64408247-64408292/ZNF117 (+43144), ZNF273 (+44650); chr7:65878502-65878629/KCTD7 (−2
15302), TPST1 (+208307); chr7:65958470-65958500/KCTD7 (−135383), TPST1 (+288226); chr7:66120093-661
20114/RABGEF1 (−84879), KCTD7 (+26236); chr7:66386062-66386085/C7orf42 (−129); chr7:66767625-667676
77/TYW1 (+305834); chr7:66767963-66768025/TYW1 (+306177); chr7:69064772-69064833/AUTS2 (+898); chr
7:70596416-70596453/WBSCR17 (−1354); chr7:70596800-70596850/WBSCR17 (−964); chr7:70597090-705973
50/WBSCR17 (−569); chr7:70597914-70597990/WBSCR17 (+163); chr7:70598124-70598169/WBSCR17 (+358);
chr7:71800757-71800933/CALN1 (+76515); chr7:71801105-71801131/CALN1 (+76242); chr7:71801306-718
02206/CALN1 (+75604); chr7:71802347-71802389/CALN1 (+74992); chr7:71802523-71802577/CALN1 (+7481
0); chr7:72838573-72838707/FZD9 (−9469), FKBP6 (+96340); chr7:72848393-72848420/FZD9 (+298); chr7:730
21661-73021710/TBL2 (−28673), MLXIPL (+17184); chr7:73085335-73085412/VPS37D (+3200), DNAJC30 (+1
2407); chr7:73118155-73118233/STX1A (+15823), WBSCR22 (+20296); chr7:73121598-73121701/STX1A (+1
2367), WBSCR22 (+23752); chr7:73130446-73130520/STX1A (+3534), WBSCR22 (+32585); chr7:73183240-7
3183299/ABHD11 (−30080), CLDN3 (+1330); chr7:73508272-73508421/EIF4H (−80359), LIMK1 (+10240); chr7:
73508993-73509072/EIF4H (−79673), LIMK1 (+10926); chr7:73589073-73589126/EIF4H (+394); chr7:73621109-
73621148/LAT2 (−2958); chr7:73720763-73720869/GTF2IRD1 (−147304), CLIP2 (+17011); chr7:73721259-737
21314/GTF2IRD1 (−146833), CLIP2 (+17482); chr7:73721512-73721623/GTF2IRD1 (−146552), CLIP2 (+17763);
chr7:73727026-73727107/GTF2IRD1 (−141053), CLIP2 (+23262); chr7:73894830-73894881/GTF2I (−177174),
GTF2IRD1 (+26736); chr7:74489091-74489136/WBSCR16 (+585); chr7:74572962-74573002/NCF1C (+14834),
GTF2IRD2B (+64635); chr7:74988406-74988459/STAG3L1 (−2809), PMS2L2 (−204); chr7:75039556-75039597/
TRIM73 (+14674), POM121C (+75991); chr7:75115314-75115365/POM121C (+228); chr7:75156795-7515692
3/POM121C (−41291), HIP1 (+211424); chr7:75508417-75508482/RHBDD2 (+133); chr7:75623792-75623857/T
MEM120A (+167); chr7:75677242-75677311/MDH2 (−116), STYXL1 (+44); chr7:75796280-75796315/HSPB1
(−135577), MDH2 (+118905); chr7:75889217-75889292/HSPB1 (−42620), MDH2 (+211862); chr7:75896686-7589
6723/HSPB1 (−35170), MDH2 (+219312); chr7:79083035-79083190/MAGI2 (−223); chr7:82072562-82072646/C
ACNA2D1 (+427); chr7:82073101-82073162/CACNA2D1 (−101); chr7:84814932-84814985/SEMA3D (−63712);
chr7:84815227-84815396/SEMA3D (−64065); chr7:86274117-86274257/GRM3 (+957); chr7:86849087-868491
61/C7orf23 (−93); chr7:87505883-87505904/SLC25A40 (−202), DBF4 (+350); chr7:88388168-88388246/ZNF804
B (−546); chr7:88388902-88389046/ZNF804B (+221); chr7:89748673-89748718/STEAP1 (−34993); chr7:897839
28-89784042/STEAP1 (+296); chr7:89840890-89841066/STEAP2 (−22); chr7:89841172-89841213/STEAP2 (+1
93); chr7:91763244-91763301/CYP51A1 (+567); chr7:92219703-92219724/RBM48 (+61627), CDK6 (+246227);
chr7:92672830-92672879/CDK6 (−206914), SAMD9 (+74481); chr7:93204232-93204298/CALCR (−223); chr7:9
3519766-93519854/TFPI2 (+255); chr7:94023699-94023821/COL1A2 (−113); chr7:95026065-95026115/PON1
(−72206), PON2 (+38294); chr7:96627049-96627151/SHFM1 (−287897), DLX6 (−8190); chr7:96635452-9663548
6/DLX6 (+179); chr7:96635590-96635619/DLX6 (+315); chr7:96635972-96636033/DLX6 (+713); chr7:9665009
5-96650275/DLX5 (+3958), DLX6 (+14895); chr7:96650437-96650751/DLX5 (+3549), DLX6 (+15304); chr7:966
51077-96651136/DLX5 (+3036), DLX6 (+15817); chr7:96651963-96652017/DLX5 (+2153), DLX6 (+16700); chr
7:96653820-96653862/DLX5 (+302); chr7:97361423-97361520/TAC1 (+201); chr7:97601189-97601265/ASNS
(−99373), OCM2 (+18189); chr7:97601409-97601419/ASNS (−99560), OCM2 (+18002); chr7:97736966-9773702
9/LMTK2 (+801); chr7:98100069-98100100/NPTX2 (−146512), BAIAP2L1 (−69658); chr7:98245732-98245856/N
PTX2 (−803); chr7:98246079-98246304/NPTX2 (−405); chr7:98246508-98246533/NPTX2 (−76); chr7:98248995-
98249073/NPTX2 (+2437), TMEM130 (+218639); chr7:98467415-98467669/TMEM130 (+131); chr7:99007150-
99007197/PDAP1 (−869), BUD31 (+573); chr7:99097194-99097277/ZNF394 (+641); chr7:99097870-99097908/
ZKSCAN5 (−4384), ZNF394 (−12); chr7:99155814-99155931/ZNF655 (−575); chr7:99177663-99177694/ZNF498
(−36892), ZNF655 (+21231); chr7:99214144-99214185/ZNF498 (−406); chr7:99595336-99595462/ZKSCAN1 (−1
7820), AZGP1P1 (+17014); chr7:99647766-99647824/ZSCAN21 (+378); chr7:99678846-99678879/ZNF3 (+508);
chr7:99698537-99698621/AP4M1 (−551), MCM7 (+848); chr7:99869451-99869511/PILRB (−86145), PVRIG
(+52609); chr7:99869935-99869970/PILRB (−85673), PVRIG (+53081); chr7:99933700-99933760/PILRB (−21896),
PVRIG (+116858); chr7:100025885-100025919/MEPCE (−1352), ZCWPW1 (+400); chr7:100028353-1000284
24/ZCWPW1 (−2087), MEPCE (+1135); chr7:100203471-100203505/MOSPD3 (−6626), PCOLCE (+3606); chr7:
100210560-100210615/MOSPD3 (+474); chr7:100253842-100253997/ACTL6B (+164); chr7:100273159-10027
3178/GNB2 (+1806), GIGYF1 (+13701); chr7:100318165-100318208/EPO (−236); chr7:100426264-100426321/
EPHB4 (−1150); chr7:100426908-100427000/EPHB4 (−1811); chr7:100463780-100463824/TRIP6 (−1148); chr7:
100463954-100463995/TRIP6 (−975); chr7:100817767-100817962/VGF (−9013), NAT16 (+5692); chr7:1008233
51-100823380/NAT16 (+191); chr7:100845201-100845248/MOGAT3 (−923); chr7:100845427-100845444/MOG
AT3 (−1134); chr7:100882612-100882698/CLDN15 (−1428); chr7:100945932-100946009/FIS1 (−57600), RABL5
(+19122); chr7:100946150-100946299/FIS1 (−57854), RABL5 (+18868); chr7:100946666-100946718/FIS1 (−58
321), RABL5 (+18401); chr7:101007361-101007396/EMID2 (+1257), MYL10 (+265197); chr7:101459514-1014
59543/CUX1 (−1353); chr7:101928419-101928559/SH2B2 (+84); chr7:102066305-102066429/ORAI2 (−7629), P
RKRIP1 (+29563); chr7:102937787-102937865/PMPCB (−47); chr7:102987935-102988020/DNAJC2 (−2658), P
SMC2 (+7); chr7:103629795-103630053/RELN (+39); chr7:103630184-103630270/RELN (−264); chr7:1039687
31-103968915/LHFPL3 (−281); chr7:103969522-103969562/LHFPL3 (+438); chr7:103969679-103969693/LHFP
L3 (+582); chr7:104653187-104653230/MLL5 (−1428); chr7:104654057-104654083/MLL5 (−567); chr7:1053194
45-105319476/CDHR3 (−284196), EFCAB10 (−97485); chr7:106808481-106808524/HBP1 (−903); chr7:1068091
66-106809218/HBP1 (−214); chr7:107220642-107220679/BCAP29 (−543); chr7:108095578-108095780/NRCAM
(−215065), PNPLA8 (+70959); chr7:108210111-108210172/DNAJB9 (−47), THAP5 (+25); chr7:112429912-1124
30003/TMEM168 (+520); chr7:112430185-112430224/TMEM168 (+273); chr7:112725919-112725945/GPR85
(+610); chr7:113723284-113723338/FOXP2 (−331741), PPP1R3A (−164229); chr7:114562277-114562326/MDFI
C (+93); chr7:116165107-116165155/CAV1 (+292); chr7:116593242-116593274/ST7 (−123); chr7:116963147-1
16963330/WNT2 (+104); chr7:116964721-116964798/WNT2 (−1417); chr7:119913385-119913416/KCND2 (−32
1); chr7:120590708-120590732/ING3 (−97); chr7:120968912-120968996/WNT16 (−136); chr7:120970703-12097
0733/WNT16 (+1628), FAM3C (+65704); chr7:121036286-121036310/FAM3C (+124); chr7:121940245-121940
433/AASS (−155995), FEZF1 (+4226); chr7:121943989-121944176/FEZF1 (+482); chr7:121946983-121947097/
FEZF1 (−2475); chr7:121950265-121950428/FEZF1 (−5782), RNF133 (+388861); chr7:121950553-121950994/F
EZF1 (−6209), RNF133 (+388434); chr7:121952011-121952043/FEZF1 (−7462), RNF133 (+387181); chr7:1219
56582-121956954/FEZF1 (−12203), RNF133 (+382440); chr7:121957077-121957253/FEZF1 (−12600), RNF133
(+382043); chr7:123197660-123197705/NDUFA5 (+275); chr7:124404207-124404299/TMEM229A (−730730),
GPR37 (+1428); chr7:124404498-124404543/TMEM229A (−730998), GPR37 (+1160); chr7:124569785-124569
848/POT1 (+220); chr7:127031779-127031863/ZNF800 (+946); chr7:127176902-127177061/ZNF800 (−144215),
GCC1 (+48672); chr7:127291954-127292077/SND1 (−186); chr7:127292265-127292290/SND1 (+76); chr7:12
7671173-127671260/LRRC4 (−215); chr7:127671719-127671742/LRRC4 (−729); chr7:127671916-127672078/L
RRC4 (−995); chr7:127672382-127672576/LRRC4 (−1477); chr7:127841273-127841338/LRRC4 (−170304), LEP
(−40025); chr7:127841497-127841625/LRRC4 (−170559), LEP (−39770); chr7:127841926-127841995/LRRC4
(−170959), LEP (−39370); chr7:127881207-127881292/LEP (−81); chr7:127991574-127991607/RBM28 (−7629), P
RRT4 (+10148); chr7:127991743-127991787/RBM28 (−7803), PRRT4 (+9974); chr7:127991923-127992044/RB
M28 (−8022), PRRT4 (+9755); chr7:127992319-127992371/RBM28 (−8383), PRRT4 (+9394); chr7:128000952-1
28001006/PRRT4 (+760); chr7:128001317-128001327/PRRT4 (+417); chr7:128171222-128171234/CALU (−20
8118), METTL2B (+54445); chr7:128337595-128337787/CALU (−41655), METTL2B (+220908); chr7:12838019
1-128380234/CALU (+867); chr7:128430882-128430922/CCDC136 (−1148); chr7:128471057-128471125/FLNC
(+608); chr7:128530932-128531156/KCP (+19729), ATP6V1F (+28187); chr7:128550363-128550659/KCP (+2
62); chr7:128555706-128555724/KCP (−4942); chr7:128555996-128556139/IRF5 (−21926), KCP (−5295); chr7:1
28578269-128578324/IRF5 (+303); chr7:128580152-128580229/IRF5 (+2197), TNPO3 (+115036); chr7:128580
708-128580791/IRF5 (+2756), TNPO3 (+114477); chr7:128694922-128694980/TNPO3 (+276); chr7:128809071-
128809124/SMO (−19615), TSPAN33 (+24386); chr7:129142001-129142044/NRF1 (−109532), FAM40B (+677
49); chr7:129251911-129251952/NRF1 (+377); chr7:129418616-129418755/NRF1 (+167131), UBE2H (+17411
4); chr7:129419286-129419320/NRF1 (+167748), UBE2H (+173497); chr7:129423013-129423034/UBE2H (+16
9776), NRF1 (+171469); chr7:129424072-129424132/UBE2H (+168698), NRF1 (+172547); chr7:129691108-12
9691179/ZC3HC1 (+89); chr7:129781009-129781096/ZC3HC1 (−89820), TMEM209 (+64285); chr7:129781221-
129781458/ZC3HC1 (−90107), TMEM209 (+63998); chr7:130125975-130126111/CEP41 (−45189), MEST (−585
6); chr7:130417938-130417976/KLF14 (+931); chr7:130418415-130418452/KLF14 (+454); chr7:130418938-13
0419012/KLF14 (−87); chr7:130790809-130790864/KLF14 (−371949), MKLN1 (−221758); chr7:131449201-1314
49339/PODXL (−207894), PLXNA4 (+812053); chr7:131594418-131594430/PODXL (−353048), PLXNA4 (+6668
99); chr7:132261208-132261256/PLXNA4 (+91); chr7:132261505-132261534/PLXNA4 (−197); chr7:132937810-
132937849/EXOC4 (+7); chr7:134143587-134143700/AKR1B1 (+244); chr7:134331238-134331322/BPGM (−25
1); chr7:134851452-134851581/C7orf49 (+4061), TMEM140 (+18751); chr7:135347477-135347496/C7orf73
(+266); chr7:136554026-136554103/CHRM2 (+233); chr7:136554581-136554625/CHRM2 (+771); chr7:13655500
1-136555078/CHRM2 (+1208), PTN (+473506); chr7:136555842-136556012/CHRM2 (+2095), PTN (+472619);
chr7:137531212-137531262/DGKI (+372); chr7:137531889-137531908/DGKI (−290); chr7:137532188-1375323
73/DGKI (−672); chr7:138144836-138144901/TRIM24 (−210); chr7:139168614-139168658/KLRG2 (−179); chr7:1
39208138-139208186/CLEC2L (−512); chr7:139208570-139208614/CLEC2L (−82); chr7:139875886-139875926/
JHDM1D (+835); chr7:140097863-140097920/SLC37A3 (+419); chr7:140098254-140098296/SLC37A3 (+36);
chr7:140178831-140178857/MKRN1 (+525); chr7:140339868-140339951/MKRN1 (−160541), ADCK2 (−33043);
chr7:140340233-140340368/MKRN1 (−160932), ADCK2 (−32652); chr7:140373597-140373662/ADCK2 (+677);
chr7:140772676-140772716/AGK (−478382), MRPS33 (−57915); chr7:141400881-141400939/WEE2 (−7243), A
GK (+149832); chr7:142494834-142495033/EPHB6 (−57858), TRY6 (+16177); chr7:142495162-142495198/EP
HB6 (−57612), TRY6 (+16423); chr7:143058758-143058840/ZYX (−19561), CLCN1 (+45580); chr7:143105627-1
43105688/EPHA1 (+327); chr7:143105827-143105844/EPHA1 (+149); chr7:145813391-145813403/CNTNAP2
(−56); chr7:145813721-145813945/CNTNAP2 (+380); chr7:148580759-148580784/EZH2 (+669); chr7:1485811
58-148581192/EZH2 (+266); chr7:148762583-148762640/PDIA4 (−36830), ZNF786 (+25257); chr7:148822432-
148822493/ZNF425 (+975); chr7:148843497-148843554/ZNF398 (−1034); chr7:149112249-149112420/ZNF777
(+45718), AF035281 (+121045); chr7:149120124-149120164/ZNF777 (+37909), AF035281 (+128854); chr7:14
9120274-149120378/ZNF777 (+37727), AF035281 (+129036); chr7:149194611-149194634/ZNF746 (+275); chr
7:149321825-149321871/ZNF746 (−126950), KRBA1 (−90300); chr7:149389723-149389934/ZNF746 (−194931),
KRBA1 (−22319); chr7:149411728-149411834/KRBA1 (−367); chr7:149744402-149744461/ATP6V0E2 (+17437
5), ACTR3C (+276326); chr7:149744799-149744950/ATP6V0E2 (+174818), ACTR3C (+275883); chr7:1497450
87-149745123/ATP6V0E2 (+175048), ACTR3C (+275653); chr7:149745590-149745672/ATP6V0E2 (+175574),
ACTR3C (+275127); chr7:149746098-149746261/ATP6V0E2 (+176123), ACTR3C (+274578); chr7:149746380-
149746404/ATP6V0E2 (+176335), ACTR3C (+274366); chr7:149916903-149916914/ACTR3C (+103849), ATP
6V0E2 (+346852); chr7:149917147-149917321/ACTR3C (+103524), ATP6V0E2 (+347177); chr7:149917469-14
9917628/ACTR3C (+103209), ATP6V0E2 (+347492); chr7:149917824-149917888/ACTR3C (+102902), ATP6V
0E2 (+347799); chr7:149918191-149918262/ACTR3C (+102531), ATP6V0E2 (+348170); chr7:150020205-1500
20224/ACTR3C (+543); chr7:150020654-150020688/ACTR3C (+87); chr7:150038066-150038171/RARRES2
(+644); chr7:150038343-150038597/RARRES2 (+293); chr7:150066866-150067012/ZNF775 (−9467), REPIN1
(+1060); chr7:150621704-150621763/KCNH2 (+53668), ABP1 (+72161); chr7:150644526-150644639/KCNH2 (+3
0819), ABP1 (+95010); chr7:150652973-150653033/KCNH2 (+22399), ABP1 (+103430); chr7:150655182-1506
55624/KCNH2 (+19999), ABP1 (+105830); chr7:150657089-150657247/KCNH2 (+18234), ABP1 (+107595); chr
7:150710632-150710694/ATG9B (+10923), NOS3 (+22519); chr7:150756396-150756434/CDK5 (−1363), SLC4
A2 (−242); chr7:150783976-150784035/TMUB1 (−3386), AGAP3 (+180); chr7:150786298-150786371/AGAP3
(+2509), GBX1 (+78532); chr7:150812783-150812802/AGAP3 (+28967), GBX1 (+52074); chr7:150864397-15086
4610/GBX1 (+363); chr7:150924255-150924302/ABCF2 (+38); chr7:150942862-150942899/SMARCD3 (+2868),
CHPF2 (+13296); chr7:150949040-150949068/SMARCD3 (−3305); chr7:151106566-151106589/NUB1 (+6773
1), RHEB (+110432); chr7:151106989-151107395/NUB1 (+68345), RHEB (+109818); chr7:151145381-1511454
11/RHEB (+71614), NUB1 (+106549); chr7:151216199-151216261/RHEB (+780); chr7:151723144-151723167/
GALNT11 (+378); chr7:152132588-152132607/MLL3 (+492); chr7:152132972-152133002/MLL3 (+103); chr7:1
52133842-152133859/MLL3 (−761); chr7:152622514-152622545/ACTR3B (+165679); chr7:152623161-152623
174/ACTR3B (+166317); chr7:153445180-153445315/DPP6 (−304529), ACTR3B (+988397); chr7:153445588-1
53446175/DPP6 (−303895), ACTR3B (+989031); chr7:153446329-153446374/DPP6 (−303425), ACTR3B (+989
501); chr7:153446650-153446725/DPP6 (−303089), ACTR3B (+989837); chr7:153583530-153583631/DPP6 (−1
66196); chr7:153585250-153585293/DPP6 (−164505); chr7:153585396-153585601/DPP6 (−164278); chr7:1537
48430-153748451/DPP6 (−1336); chr7:153748649-153748846/DPP6 (−1029); chr7:153749132-153749164/DPP
6 (−629); chr7:153750043-153750249/DPP6 (+369); chr7:154002010-154002083/DPP6 (+252270), PAXIP1 (+7
92635); chr7:154705826-154705874/PAXIP1 (+88832), DPP6 (+956073); chr7:155137148-155137202/EN2 (−1
13649), INSIG1 (+47689); chr7:155167090-155167174/EN2 (−83692), INSIG1 (+77646); chr7:155167661-15516
7833/EN2 (−83077), INSIG1 (+78261); chr7:155175154-155175246/EN2 (−75624), INSIG1 (+85714); chr7:1552
42309-155242379/EN2 (−8480), INSIG1 (+152858); chr7:155242519-155242699/EN2 (−8215), INSIG1 (+15312
3); chr7:155243534-155243755/EN2 (−7179), INSIG1 (+154159); chr7:155246744-155246858/EN2 (−4023); chr
7:155247480-155247650/EN2 (−3259); chr7:155250304-155250323/EN2 (−510); chr7:155251259-155251346/E
N2 (+479); chr7:155251550-155251661/EN2 (+782); chr7:155251855-155251890/RBM33 (−185330), EN2 (+10
49); chr7:155252262-155252316/RBM33 (−184914), EN2 (+1465); chr7:155256233-155256268/RBM33 (−18095
2), EN2 (+5427); chr7:155258864-155258914/RBM33 (−178314), EN2 (+8065); chr7:155259078-155259119/RB
M33 (−178104), EN2 (+8275); chr7:155259623-155259833/RBM33 (−177475), EN2 (+8904); chr7:155259958-15
5260038/RBM33 (−177205), EN2 (+9174); chr7:155260891-155261070/RBM33 (−176222), EN2 (+10157); chr7:
155284202-155284261/RBM33 (−152971), EN2 (+33408); chr7:155302896-155302963/RBM33 (−134273), EN2
(+52106); chr7:155556566-155556651/SHH (+48358), RBM33 (+119406); chr7:155579929-155579984/SHH
(+25010), RBM33 (+142754); chr7:155595588-155595991/SHH (+9177), RBM33 (+158587); chr7:155596657-155
596712/SHH (+8282), RBM33 (+159482); chr7:155597718-155597775/SHH (+7220), RBM33 (+160544); chr7:1
55598257-155598353/SHH (+6662), RBM33 (+161102); chr7:155598827-155598940/SHH (+6083), RBM33 (+1
61681); chr7:155599312-155599362/SHH (+5630), RBM33 (+162134); chr7:155606727-155606913/SHH (−185
3); chr7:156400778-156400857/SHH (−795851), C7orf13 (+32530); chr7:156400984-156401043/SHH (−796047),
C7orf13 (+32334); chr7:156409513-156409727/SHH (−804653), C7orf13 (+23728); chr7:156701662-15670179
3/NOM1 (−40689), LMBR1 (−15826); chr7:156795355-156795401/MNX1 (+7969), NOM1 (+52961); chr7:156795
636-156795899/MNX1 (+7579), NOM1 (+53351); chr7:156796835-156797005/MNX1 (+6427), NOM1 (+54503);
chr7:156798435-156798526/MNX1 (+4866), NOM1 (+56064); chr7:156799147-156799290/MNX1 (+4128), NO
M1 (+56802); chr7:156801872-156801943/MNX1 (+1439), NOM1 (+59491); chr7:156809513-156809643/UBE3
C (−122077), MNX1 (−6231); chr7:156810522-156810597/UBE3C (−121095), MNX1 (−7213); chr7:156810801-15
6811302/UBE3C (−120603), MNX1 (−7705); chr7:156813826-156813986/UBE3C (−117749), MNX1 (−10559); chr
7:156814708-156814783/UBE3C (−116909), MNX1 (−11399); chr7:156871153-156871282/MNX1 (−67871), UBE
3C (−60437); chr7:156931564-156931622/UBE3C (−62); chr7:157295737-157295768/DNAJB6 (+166043); chr7:
157361653-157361751/DNAJB6 (+231992); chr7:157378194-157378268/DNAJB6 (+248521); chr7:157461396-
157461465/DNAJB6 (+331721), PTPRN2 (+919051); chr7:157476974-157476994/DNAJB6 (+347274), PTPRN
2 (+903498); chr7:157477555-157477604/DNAJB6 (+347870), PTPRN2 (+902902); chr7:157478019-15747840
7/DNAJB6 (+348503), PTPRN2 (+902269); chr7:157478586-157478638/DNAJB6 (+348902), PTPRN2 (+90187
0); chr7:157481250-157481533/DNAJB6 (+351682), PTPRN2 (+899090); chr7:157481657-157481741/DNAJB6
(+351989), PTPRN2 (+898783); chr7:157482074-157482200/DNAJB6 (+352427), PTPRN2 (+898345); chr7:15
7484486-157484507/DNAJB6 (+354787), PTPRN2 (+895985); chr7:157485602-157485649/DNAJB6 (+355916),
PTPRN2 (+894856); chr7:157486082-157486204/DNAJB6 (+356433), PTPRN2 (+894339); chr7:157486415-1
57486475/DNAJB6 (+356735), PTPRN2 (+894037); chr7:157486603-157486656/DNAJB6 (+356920), PTPRN2
(+893852); chr7:158110682-158110727/PTPRN2 (+269777), DNAJB6 (+980995); chr7:158203537-158203631/
PTPRN2 (+176898); chr7:158362648-158362944/PTPRN2 (+17686); chr7:158380489-158380510/PTPRN2 (−1
8); chr7:158497157-158497187/NCAPG2 (+348); chr7:158505106-158505231/NCAPG2 (−7649), ESYT2 (+117
150); chr7:158589457-158589519/NCAPG2 (−91968), ESYT2 (+32831); chr7:158620154-158620202/NCAPG2
(−122658), ESYT2 (+2141); chr7:158620858-158620911/NCAPG2 (−123365), ESYT2 (+1434); chr7:158765341-
158765441/ESYT2 (−143072), VIPR2 (+172258); chr7:158937091-158937202/VIPR2 (+502); chr7:158937375-1
58937527/VIPR2 (+198); chr7:158938046-158938131/VIPR2 (−440); chr8:357052-357083/FBXO25 (+260); chr8:
494270-494636/C8orf42 (+878); chr8:494828-494853/C8orf42 (+490); chr8:495165-495223/C8orf42 (+137); ch
r8:495523-495635/C8orf42 (−248); chr8:495878-495952/C8orf42 (−584); chr8:638495-638542/DLGAP2 (−81105
0), C8orf42 (−143188); chr8:686980-687070/DLGAP2 (−762544), C8orf42 (−191694); chr8:687218-687318/DLG
AP2 (−762301), C8orf42 (−191937); chr8:687734-687773/DLGAP2 (−761815), C8orf42 (−192423); chr8:687976-6
88026/DLGAP2 (−761568), C8orf42 (−192670); chr8:688385-688498/DLGAP2 (−761127), C8orf42 (−193111); chr
8:1950369-1950480/MYOM2 (−42733), ARHGEF10 (+178276); chr8:1972039-1972132/MYOM2 (−21072), ARH
GEF10 (+199937); chr8:2212437-2212457/MYOM2 (+219289); chr8:2418942-2418986/MYOM2 (+425806); chr
8:3573196-3573310/NONE; chr8:4754189-4754312/CSMD1 (+98077); chr8:4849263-4849296/CSMD1 (+3048);
chr8:4850323-4850418/CSMD1 (+1957); chr8:4851750-4851780/CSMD1 (+563); chr8:6264109-6264135/MCP
H1 (+9); chr8:6748046-6748180/DEFB1 (−12584), DEFA6 (+35485); chr8:6949700-6949751/FAM90A13 (−1647
00), DEFA5 (−35467); chr8:6950023-6950078/FAM90A13 (−164375), DEFA5 (−35792); chr8:7923176-7923208/L
OC100132396 (+139333), SGK223 (+316065); chr8:8559519-8559542/CLDN23 (−135); chr8:8559844-8559899/
CLDN23 (+206); chr8:8654846-8654935/ERI1 (−205423), CLDN23 (+95225); chr8:9009011-9009104/PPP1R3B
(−838); chr8:9755803-9755972/MSRA (−155942), TNKS (+342443); chr8:9756284-9756486/MSRA (−155445), T
NKS (+342940); chr8:9762690-9762751/MSRA (−149109), TNKS (+349276); chr8:9763762-9763787/MSRA (−1
48055), TNKS (+350330); chr8:10192379-10192409/PRSS55 (−190662), MSRA (+280564); chr8:10574224-105
74356/RP1L1 (−61673), SOX7 (+13732); chr8:10586829-10586922/RP1L1 (−74259), SOX7 (+1146); chr8:10588
311-10588350/SOX7 (−309); chr8:10588596-10588640/SOX7 (−596); chr8:10588950-10589008/SOX7 (−957); c
hr8:10590108-10590176/SOX7 (−2120); chr8:10591034-10591057/SOX7 (−3024); chr8:10697151-10697188/SO
X7 (−109148), XKR6 (+361705); chr8:10708311-10708327/SOX7 (−120297), XKR6 (+350556); chr8:11058167-1
1058198/XKR6 (+692); chr8:11059175-11059210/XKR6 (−318); chr8:11205422-11205445/BLK (−146087), SLC3
5G5 (+16939); chr8:11324523-11324585/BLK (−26967), SLC35G5 (+136059); chr8:11470848-11470946/GATA
4 (−90820), BLK (+119376); chr8:11471148-11471176/GATA4 (−90555), BLK (+119641); chr8:11537024-11537
156/GATA4 (−24627), BLK (+185569); chr8:11537589-11537704/GATA4 (−24070), BLK (+186126); chr8:115378
54-11538169/GATA4 (−23705), BLK (+186491); chr8:11538629-11538785/GATA4 (−23010), BLK (+187186); chr
8:11540730-11540851/GATA4 (−20926), BLK (+189270); chr8:11555167-11555473/GATA4 (−6397), BLK (+203
799); chr8:11558138-11558196/GATA4 (−3550); chr8:11558927-11558989/GATA4 (−2759); chr8:11559665-115
59706/GATA4 (−2031); chr8:11562196-11562235/GATA4 (+499); chr8:11565422-11565504/C8orf49 (−53302),
GATA4 (+3746); chr8:11565889-11566005/C8orf49 (−52818), GATA4 (+4230); chr8:11566417-11566713/C8orf
49 (−52200), GATA4 (+4848); chr8:11627452-11627472/NEIL2 (+290); chr8:11660236-11660278/FDFT1 (+67);
chr8:11872600-11872660/DEFB134 (−18870), LOC100133267 (+56626); chr8:12517074-12517105/LONRF1
(+95902), ZNF705A (+308949); chr8:12612625-12612679/LONRF1 (+340); chr8:13424253-13424344/C8orf48 (−5
3); chr8:15094277-15094366/SGCZ (+1470); chr8:15094567-15094645/SGCZ (+1186); chr8:15095084-150951
36/SGCZ (+682); chr8:15397664-15397685/TUSC3 (+79); chr8:15893837-15893888/MSR1 (+156437), TUSC3
(+496267); chr8:17014621-17014634/ZDHHC2 (+792); chr8:17103754-17103791/VPS37A (−628), CNOT7 (+61
4); chr8:17270676-17270878/MTMR7 (+263); chr8:17270997-17271029/MTMR7 (+27); chr8:17509194-175093
05/PDGFRL (+75308), MTUS1 (+149176); chr8:17780478-17780528/PCM1 (+137); chr8:17941981-17942067/
ASAH1 (−145); chr8:18245116-18245164/NAT2 (−3615); chr8:18871974-18872035/PSD3 (−809); chr8:19614781-
19614842/CSGALNACT1 (−74718), INTS10 (−60106); chr8:19674267-19674312/INTS10 (−628); chr8:19797609-
19797677/LPL (+1061), SLC18A1 (+243074); chr8:20092455-20092509/LZTS1 (+20321), ATP6V1B2 (+37778);
chr8:20160710-20160990/LZTS1 (−48047); chr8:20161651-20161666/LZTS1 (−48856); chr8:21067589-210676
59/LZTS1 (−954821), GFRA2 (+578722); chr8:21549817-21549914/GFRA2 (+96480); chr8:21646517-21646557/
GFRA2 (−191); chr8:21647584-21647675/GFRA2 (−1284); chr8:21777844-21777878/XPO7 (+681); chr8:21905
541-21905571/EPB49 (−6772), FGF17 (+5128); chr8:21905672-21905708/EPB49 (−6638), FGF17 (+5262); chr8:
21924612-21924666/EPB49 (+12311), NUDT18 (+42817); chr8:21990917-21990970/HR (−2379); chr8:220216
44-22021665/BMP1 (−998); chr8:22409218-22409253/SORBS3 (−15); chr8:22422687-22422738/PDLIM2 (−1354
1), SORBS3 (+13462); chr8:22436828-22436855/PDLIM2 (+588); chr8:22547751-22547867/BIN3 (−21148), EG
R3 (+3006); chr8:22548315-22548335/BIN3 (−21664), EGR3 (+2490); chr8:22548665-22548720/BIN3 (−22032),
EGR3 (+2122); chr8:22548824-22548881/BIN3 (−22192), EGR3 (+1962); chr8:22561217-22561395/EGR3 (−10
491), PEBP4 (+224115); chr8:23020906-23020932/TNFRSF10D (+621); chr8:23021131-23021192/TNFRSF10
D (+378); chr8:23082657-23082729/TNFRSF10A (−13); chr8:23103785-23103817/R3HCC1 (−41811), CHMP7
(+2651); chr8:23261247-23261271/LOXL2 (+463); chr8:23540437-23540467/NKX3-1 (−2); chr8:23559602-2355
9665/NKX3-1 (−19184), NKX2-6 (+4477); chr8:23563053-23563265/NKX2-6 (+952); chr8:23564024-23564192/
NKX2-6 (+3); chr8:23564669-23564702/NKX2-6 (−575); chr8:23566855-23566900/NKX2-6 (−2767); chr8:235672
14-23567311/NKX2-6 (−3152); chr8:23567583-23567657/NKX2-6 (−3509); chr8:23584017-23584093/NKX2-6
(−19944), STC1 (+128265); chr8:24770362-24770413/NEFM (−886); chr8:24771272-24771337/NEFM (+31); chr8:
24771575-24771632/NEFM (+330); chr8:24799454-24799514/NEFL (+14647), NEFM (+28210); chr8:24813464-
24813618/NEFL (+590); chr8:24813894-24814010/NEFL (+179); chr8:24816606-24816694/NEFL (−2519); chr8:
25897314-25897560/EBF2 (+5203), CDCA2 (+580924); chr8:25898123-25898395/EBF2 (+4381), CDCA2 (+58
1746); chr8:25899046-25899186/EBF2 (+3524), CDCA2 (+582603); chr8:25900693-25900780/EBF2 (+1903), C
DCA2 (+584224); chr8:25905629-25905695/EBF2 (−3022); chr8:25905993-25906080/EBF2 (−3397); chr8:25906
974-25907061/EBF2 (−4378); chr8:25907751-25908063/BNIP3L (−332616), EBF2 (−5267); chr8:25908425-2590
8502/BNIP3L (−332059), EBF2 (−5824); chr8:26240429-26240445/BNIP3L (−86); chr8:26241035-26241050/BNI
P3L (+520); chr8:26298728-26298836/BNIP3L (+58259), PNMA2 (+72701); chr8:26721765-26721919/ADRA1A
(+1080), DPYSL2 (+350133); chr8:26722581-26722717/ADRA1A (+273); chr8:26724440-26724484/ADRA1A
(−1540); chr8:27221338-27221422/PTK2B (+38384), CHRNA2 (+115433); chr8:27695407-27695447/PBK (−78); c
hr8:27758917-27758956/PBK (−63588), SCARA5 (+91432); chr8:28269963-28269978/FZD3 (−81751), ZNF395
(−25994); chr8:28479673-28479739/EXTL3 (−79447), FZD3 (+127984); chr8:29120598-29120620/KIF13B (+1);
chr8:30769783-30769822/PPP2CB (−99451), PURG (+120514); chr8:30770110-30770157/PPP2CB (−99782), P
URG (+120183); chr8:31498015-31498153/NRG1 (−907644), WRN (+607306); chr8:32406237-32406351/NRG1
(+566); chr8:33342426-33342459/MAK16 (−242); chr8:33424714-33424756/RNF122 (−92); chr8:35092703-350
92993/UNC5D (−127); chr8:35093160-35093225/UNC5D (+218); chr8:35093543-35093597/UNC5D (+595); chr
8:35093974-35094035/UNC5D (+1030); chr8:37555740-37555815/ERLIN2 (−38319), ZNF703 (+2477); chr8:37
556119-37556142/ERLIN2 (−37966), ZNF703 (+2830); chr8:37557258-37557330/ERLIN2 (−36803), ZNF703 (+3
993); chr8:37655376-37655475/GPR124 (+1025), BRF2 (+52005); chr8:37655991-37656049/GPR124 (+1619),
BRF2 (+51411); chr8:37756575-37756610/RAB11FIP1 (+422); chr8:37822529-37822569/GOT1L1 (−24902), A
DRB3 (+1635); chr8:37823532-37823789/ADRB3 (+523); chr8:37887398-37887456/EIF4EBP1 (−593); chr8:379
62789-37962832/ASH2L (−200); chr8:37963427-37963451/ASH2L (+428); chr8:38323735-38323755/FGFR1
(+1618), LETM2 (+79786); chr8:38324749-38324811/FGFR1 (+583); chr8:38412004-38412143/FGFR1 (−86711),
RNF5P1 (+46701); chr8:38508556-38508657/TACC1 (−136115), RNF5P1 (−49832); chr8:38757676-38757771/P
LEKHA2 (−1029); chr8:38759230-38759277/PLEKHA2 (+501); chr8:38831597-38832135/HTRA4 (+198); chr8:3
8853645-38853713/ADAM9 (−826), TM2D2 (+362); chr8:41165919-41166000/ZMAT4 (−410617), SFRP1 (+103
0); chr8:41166152-41166266/SFRP1 (+781); chr8:41166680-41166745/SFRP1 (+277); chr8:41166989-411670
25/SFRP1 (−17); chr8:41219115-41219207/GOLGA7 (−128920), SFRP1 (−52171); chr8:41347851-41347909/GO
LGA7 (−201); chr8:41424466-41424534/AGPAT6 (−11207), GINS4 (+37775); chr8:41425130-41425243/AGPAT
6 (−10520), GINS4 (+38462); chr8:41511107-41511147/NKX6-3 (−6252), ANK1 (+243153); chr8:41624376-4162
4412/NKX6-3 (−119519), ANK1 (+129886); chr8:41624561-41624633/NKX6-3 (−119722), ANK1 (+129683); chr8:
41655058-41655301/NKX6-3 (−150305), ANK1 (+99100); chr8:41733654-41733684/NKX6-3 (−228794), ANK1
(+20611); chr8:41753753-41753770/ANK1 (+518); chr8:42052369-42052510/PLAT (+12754), AP3M2 (+41976);
chr8:42195985-42196025/POLB (+32); chr8:42911980-42912019/FNTA (+558); chr8:43101742-43101777/HGS
NAT (+106168); chr8:43101979-43102074/HGSNAT (+106435); chr8:43102407-43102486/HGSNAT (+106855);
chr8:43102596-43102666/HGSNAT (+107039); chr8:47529111-47529145/NONE; chr8:48100757-48100826/C
EBPD (+549934); chr8:49293525-49293580/EFCAB1 (+354317), UBE2V2 (+372558); chr8:49309093-4930919
8/EFCAB1 (+338724), UBE2V2 (+388151); chr8:49309757-49309797/EFCAB1 (+338093), UBE2V2 (+388782);
chr8:49343030-49343136/EFCAB1 (+304787), UBE2V2 (+422088); chr8:49427121-49427274/EFCAB1 (+2206
72), UBE2V2 (+506203); chr8:49782886-49782922/EFCAB1 (−135034), SNAI2 (+51095); chr8:50822358-50822
494/SNTG1 (−2171); chr8:50824308-50824352/SNTG1 (−267); chr8:52812091-52812151/PCMTD1 (−386); chr8:
53477737-53477944/FAM150A (+180); chr8:53852343-53852434/NPBWR1 (−79); chr8:53852914-53853111/N
PBWR1 (+545); chr8:53853514-53853579/NPBWR1 (+1079), OPRK1 (+310647); chr8:54163350-54163405/OP
RK1 (+816); chr8:54163569-54163648/OPRK1 (+585); chr8:54164127-54164390/OPRK1 (−65); chr8:54789845-
54789885/RGS20 (+25497), TCEA1 (+145143); chr8:54791782-54791897/RGS20 (+27472), TCEA1 (+143168);
chr8:54792022-54792084/RGS20 (+27685), TCEA1 (+142955); chr8:54792671-54792701/RGS20 (+28318), T
CEA1 (+142322); chr8:54794781-54794826/RGS20 (+30436), TCEA1 (+140204); chr8:54794950-54795139/R
GS20 (+30677), TCEA1 (+139963); chr8:54933978-54934035/TCEA1 (+1001), RGS20 (+169639); chr8:549345
96-54934624/TCEA1 (+398); chr8:55366368-55366951/SOX17 (−3835); chr8:55370433-55370567/SOX17 (+5);
chr8:55370714-55370835/SOX17 (+280); chr8:55371033-55371172/SOX17 (+608); chr8:55371376-55371439/
SOX17 (+913); chr8:55371725-55371993/RP1 (−156768), SOX17 (+1364); chr8:55372172-55372416/RP1 (−156
333), SOX17 (+1799); chr8:55379231-55379295/RP1 (−149364), SOX17 (+8768); chr8:55379472-55379721/RP
1 (−149030), SOX17 (+9102); chr8:55379831-55380036/RP1 (−148693), SOX17 (+9439); chr8:55380144-55380
175/RP1 (−148467), SOX17 (+9665); chr8:55382673-55383153/RP1 (−145714), SOX17 (+12418); chr8:5601389
3-56014011/XKR4 (−1065); chr8:56014185-56014376/XKR4 (−736); chr8:56015053-56015185/XKR4 (+102); chr
8:56015662-56015749/XKR4 (+689); chr8:56986598-56986641/RPS20 (+520); chr8:57025620-57025775/MOS
(+843); chr8:57026322-57026501/MOS (+129); chr8:57069738-57069850/MOS (−43253), PLAG1 (+54065); chr
8:57358147-57358261/PENK (+389); chr8:57358392-57358448/PENK (+173); chr8:57359092-57359259/PENK
(−583); chr8:57360626-57360769/PENK (−2105); chr8:58055164-58055187/FAM110B (−851937), IMPAD1 (−148
746); chr8:58907115-58907135/FAM110B (+12); chr8:58907419-58907483/FAM110B (+338); chr8:59058261-5
9058282/UBXN2B (−265551), FAM110B (+151159); chr8:61764726-61764738/CLVS1 (−435793), CHD7 (+1734
08); chr8:61821427-61821458/CLVS1 (−379082), CHD7 (+230119); chr8:65282005-65282332/BHLHE22 (−2106
26); chr8:65282441-65282640/BHLHE22 (−210254); chr8:65282945-65283041/BHLHE22 (−209802); chr8:65283
469-65283506/BHLHE22 (−209307); chr8:65286067-65286370/BHLHE22 (−206576); chr8:65290682-65290897/
BHLHE22 (−202005); chr8:65293194-65293265/BHLHE22 (−199565); chr8:65489583-65489646/BHLHE22 (−31
80); chr8:65490709-65490762/BHLHE22 (−2059); chr8:65491101-65491294/BHLHE22 (−1597); chr8:65493955-
65494076/BHLHE22 (+1221), CYP7B1 (+217332); chr8:65711084-65711221/CYP7B1 (+195); chr8:66557337-6
6557366/MTFR1 (+464); chr8:67090453-67090473/CRH (+383); chr8:67344702-67344809/ADHFE1 (+38); chr8:
67424553-67424623/ADHFE1 (+79870), MYBL1 (+100892); chr8:67525189-67525238/MYBL1 (+266); chr8:67
525403-67525435/MYBL1 (+61); chr8:67873493-67873798/TCF24 (+1179), C8orf45 (+90662); chr8:67874051-
67874164/TCF24 (+717); chr8:67874673-67874755/TCF24 (+111); chr8:67974212-67974225/CSPP1 (−2384),
COPS5 (+343); chr8:68864471-68864545/PREX2 (−95); chr8:68864907-68864942/PREX2 (+322); chr8:692430
67-69243284/C8orf34 (+219); chr8:69243903-69243963/C8orf34 (+976); chr8:70623421-70623536/SULF1 (+83
472), SLCO5A1 (+123820); chr8:70907054-70907109/SLCO5A1 (−159783), PRDM14 (+76480); chr8:70981934-
70982262/SLCO5A1 (−234799), PRDM14 (+1464); chr8:70982567-70982850/PRDM14 (+853); chr8:70983870-
70984016/PRDM14 (−381); chr8:70984293-70984343/PRDM14 (−756); chr8:70984662-70984744/PRDM14 (−11
41); chr8:71314385-71314426/PRDM14 (−330844), NCOA2 (+1614); chr8:71519882-71519912/TRAM1 (+797);
chr8:71520638-71520665/TRAM1 (+42); chr8:72459571-72459759/EYA1 (−190686), MSC (+297066); chr8:724
59897-72459960/EYA1 (−190950), MSC (+296802); chr8:72460298-72460433/EYA1 (−191387), MSC (+296365);
chr8:72470206-72470259/EYA1 (−201254), MSC (+286498); chr8:72519460-72519475/EYA1 (−250489), MSC
(+237263); chr8:72754361-72754490/EYA1 (−485447), MSC (+2305); chr8:72755815-72756655/MSC (+496); c
hr8:72917429-72917515/MSC (−160741), TRPA1 (+70347); chr8:73163661-73163859/KCNB2 (−285866), TRPA
1 (−175941); chr8:73449955-73450026/KCNB2 (+365); chr8:74005661-74005798/C8orf84 (−223); chr8:7465909
1-74659124/STAU2 (+54); chr8:74790762-74790822/UBE2W (+318); chr8:74884260-74884301/TMEM70 (−409
6), TCEB1 (+241); chr8:76316556-76316614/HNF4G (−135618), CRISPLD1 (+419877); chr8:77594675-775947
57/ZFHX4 (+1201), PEX2 (+318564); chr8:80696058-80696101/HEY1 (−15982), MRPS28 (+246426); chr8:8080
4117-80804178/HEY1 (−124050), MRPS28 (+138358); chr8:80804290-80804306/HEY1 (−124200), MRPS28
(+138208); chr8:80941978-80942045/MRPS28 (+494); chr8:82753897-82753939/SNX16 (+603); chr8:85094841-
85094922/RALYL (−2218); chr8:85096773-85096852/RALYL (−287); chr8:85097003-85097062/RALYL (−67); chr
8:86350567-86350926/CA3 (−309); chr8:87081678-87081763/PSKH2 (+130); chr8:87520972-87521042/FAM82
B (+2); chr8:87526737-87526780/CPNE3 (+103); chr8:89340053-89340081/MMP16 (−350); chr8:89340237-893
40273/MMP16 (−538); chr8:90913978-90914036/OSGIN2 (−755); chr8:91095084-91095141/CALB1 (−6); chr8:91
803822-91803909/NECAB1 (−55); chr8:91997509-91997527/TMEM55A (+55685), NECAB1 (+193597); chr8:93
114242-93114306/RUNX1T1 (−6392), C8orf83 (+864098); chr8:94508197-94508294/C8orf83 (−529874), RBM1
2B (+244978); chr8:94752511-94752589/RBM12B (+674); chr8:94929541-94929574/PDP1 (−434); chr8:949297
59-94929775/PDP1 (−225); chr8:95651269-95651324/ESRP1 (−2067); chr8:95653240-95653290/ESRP1 (−99);
chr8:95908075-95908097/CCNE2 (−604); chr8:96037649-96037701/C8orf38 (+461); chr8:96193484-96193591/
PLEKHF2 (+47589), GDF6 (+979482); chr8:96281737-96281794/PLEKHF2 (+135817), GDF6 (+891254); chr8:
97156692-97156897/GDF6 (+16225); chr8:97157243-97157666/GDF6 (+15565); chr8:97157898-97158017/GD
F6 (+15062); chr8:97168078-97168213/GDF6 (+4874); chr8:97169620-97169708/GDF6 (+3356); chr8:9716995
6-97170053/GDF6 (+3015); chr8:97170338-97170377/GDF6 (+2662); chr8:97171265-97171317/GDF6 (+1729);
chr8:97171959-97172018/GDF6 (+1031); chr8:97172740-97172821/GDF6 (+239); chr8:97173864-97173920/
GDF6 (−872); chr8:97505763-97505801/SDC2 (−100); chr8:97506049-97506177/SDC2 (+231); chr8:97506408-
97506447/SDC2 (+546); chr8:97657088-97657174/PGCP (−368); chr8:98289868-98289922/TSPYL5 (+281); ch
r8:99058508-99058585/RPL30 (−729); chr8:99076683-99076759/RPL30 (−18903), HRSP12 (+52697); chr8:994
38719-99438739/KCNS2 (−521); chr8:99838387-99838487/STK3 (−528); chr8:99952144-99952198/OSR2 (−446
0); chr8:99952350-99952511/OSR2 (−4200); chr8:99952933-99952968/OSR2 (−3680); chr8:99954563-9995467
8/OSR2 (−2010); chr8:99959933-99959960/VPS13B (−65547), OSR2 (+3316); chr8:99960657-99960752/VPS13
B (−64789), OSR2 (+4074); chr8:99961174-99961568/VPS13B (−64123), OSR2 (+4740); chr8:99984871-99984
923/VPS13B (−40597), OSR2 (+28266); chr8:99986117-99986199/VPS13B (−39336), OSR2 (+29527); chr8:999
86527-99986791/VPS13B (−38835), OSR2 (+30028); chr8:100025727-100025796/VPS13B (+268); chr8:10002
5932-100025965/VPS13B (+455); chr8:101118491-101118658/RGS22 (−231); chr8:101571575-101571603/AN
KRD46 (+423); chr8:101733712-101733741/PABPC1 (+588); chr8:101733960-101734092/PABPC1 (+289); chr
8:102236352-102236442/GRHL2 (−268271), ZNF706 (−18437); chr8:103541174-103541252/UBR5 (−116296), O
DF1 (−22635); chr8:103750820-103750885/KLF10 (−82870), AZIN1 (+125544); chr8:103807463-103807542/KL
F10 (−139520), AZIN1 (+68894); chr8:104153562-104153681/BAALC (+701); chr8:104311026-104311076/FZD
6 (+390); chr8:105235502-105235643/TM7SF4 (−116481), RIMS2 (+722597); chr8:105235804-105235863/TM7
SF4 (−116220), RIMS2 (+722858); chr8:105478780-105479247/DPYS (+263); chr8:106331074-106331083/ZFP
M2 (−68); chr8:106331257-106331443/ZFPM2 (+203); chr8:106331974-106332003/ZFPM2 (+842); chr8:109095
437-109095505/RSPO2 (+442); chr8:109095974-109096033/RSPO2 (−91); chr8:109799740-109799888/TMEM
74 (−44); chr8:110656828-110656890/SYBU (+192); chr8:110657173-110657196/SYBU (−134); chr8:110986264-
110986328/SYBU (−329245), KCNV1 (+1125); chr8:114444394-114444481/CSMD3 (+4804); chr8:114802671-
114802717/CSMD3 (−353452); chr8:116660572-116660615/TRPS1 (+20634); chr8:116679743-116679829/TR
PS1 (+1442); chr8:117886963-117887066/RAD21 (+90); chr8:118532869-118532906/MED30 (−77); chr8:11912
3400-119123459/EXT1 (+628); chr8:119633489-119633554/EXT1 (−509464), TNFRSF11B (+330861); chr8:119
633778-119633870/EXT1 (−509766), TNFRSF11B (+330559); chr8:120428923-120428985/NOV (+402); chr8:1
20868394-120868459/DSCC1 (−257); chr8:123793264-123793324/ZHX2 (−607); chr8:124169050-124169099/W
DR67 (+84155), C8orf76 (+84563); chr8:124285963-124286033/ZHX1 (+729); chr8:124428992-124429023/WD
YHV1 (+43); chr8:124553797-124553828/FBXO32 (−320); chr8:126442040-126442109/TRIB1 (−488); chr8:1265
88124-126588227/TRIB1 (+145613), FAM84B (+982535); chr8:131058381-131058460/GSDMC (−259287), ASA
P1 (+397485); chr8:132052300-132052398/ADCY8 (+486); chr8:132052516-132052589/ADCY8 (+282); chr8:1
32054584-132054593/ADCY8 (−1754); chr8:133492111-133492127/KCNQ3 (+885); chr8:133492631-13349271
2/KCNQ3 (+332); chr8:133493752-133493782/KCNQ3 (−763); chr8:139275425-139275495/COL22A1 (+650776);
chr8:139508947-139509549/COL22A1 (+416988); chr8:139509671-139509693/COL22A1 (+416554); chr8:14
0715095-140715219/KCNK9 (+142); chr8:140715588-140715699/KCNK9 (−345); chr8:140716119-140716137/
KCNK9 (−829); chr8:140716310-140716339/KCNK9 (−1026); chr8:141404985-141405049/KCNK9 (−689718), TR
APPC9 (+63661); chr8:141467793-141467845/TRAPPC9 (+859); chr8:141521933-141521946/CHRAC1 (+543);
chr8:141552182-141552246/CHRAC1 (+30817), EIF2C2 (+93432); chr8:141608979-141609047/EIF2C2 (+366
33), CHRAC1 (+87616); chr8:141646269-141646330/EIF2C2 (−654); chr8:141849213-141849295/EIF2C2 (−203
608), PTK2 (+162158); chr8:142011063-142011091/PTK2 (+335); chr8:142046287-142046339/PTK2 (−34901),
SLC45A4 (+192360); chr8:142099939-142100059/PTK2 (−88587), SLC45A4 (+138674); chr8:142105795-14210
5835/PTK2 (−94403), SLC45A4 (+132858); chr8:142160080-142160270/PTK2 (−148763), SLC45A4 (+78498); c
hr8:142216529-142216598/PTK2 (−205152), SLC45A4 (+22109); chr8:142216898-142217015/PTK2 (−205545),
SLC45A4 (+21716); chr8:142276277-142276532/SLC45A4 (−37732), GPR20 (+100960); chr8:142297852-1422
97913/SLC45A4 (−59210), GPR20 (+79482); chr8:142411558-142411675/GPR20 (−34252), PTP4A3 (−20390); c
hr8:142412512-142412666/GPR20 (−35224), PTP4A3 (−19418); chr8:142427219-142427304/PTP4A3 (−4745); c
hr8:142428292-142428316/PTP4A3 (−3703); chr8:142528141-142528198/FLJ43860 (−10840), TSNARE1 (+956
373); chr8:142528403-142528454/FLJ43860 (−11099), TSNARE1 (+956114); chr8:142528607-142528670/FLJ4
3860 (−11309), TSNARE1 (+955904); chr8:142528782-142528834/FLJ43860 (−11478), TSNARE1 (+955735); c
hr8:142528962-142529027/FLJ43860 (−11665), TSNARE1 (+955548); chr8:143020715-143020769/FLJ43860
(−503412), TSNARE1 (+463801); chr8:143498274-143498386/BAI1 (−47047), TSNARE1 (−13787); chr8:1435096
07-143509628/BAI1 (−35759), TSNARE1 (−25075); chr8:143532510-143532541/TSNARE1 (−47983), BAI1 (−128
51); chr8:143533641-143533708/TSNARE1 (−49132), BAI1 (−11702); chr8:143545588-143545634/BAI1 (+234);
chr8:143545788-143546251/BAI1 (+643); chr8:143553067-143553170/BAI1 (+7742), ARC (+142714); chr8:143
591807-143591888/BAI1 (+46471), ARC (+103985); chr8:143592044-143592101/BAI1 (+46696), ARC (+10376
0); chr8:143592279-143592358/BAI1 (+46942), ARC (+103514); chr8:143592579-143592663/BAI1 (+47244), A
RC (+103212); chr8:143695906-143695943/ARC (−92); chr8:143762027-143762146/PSCA (+213); chr8:143809
370-143809517/C8orf55 (+823); chr8:143858820-143858864/LYNX1 (−7596), LY6D (+9166); chr8:143859020-1
43859071/LYNX1 (−7800), LY6D (+8962); chr8:143859281-143859337/LYNX1 (−8063), LY6D (+8699); chr8:144
105788-144105841/LY6E (+5913), LY6H (+135940); chr8:144161962-144162002/LY6E (+62080), LY6H (+7977
3); chr8:144241522-144241583/LY6H (+202); chr8:144241703-144241757/LY6H (+25); chr8:144349675-14434
9708/GLI4 (+85); chr8:144503225-144503284/MAFA (+9347), RHPN1 (+52230); chr8:144598828-144598953/
MAFA (−86289), ZC3H3 (+24729); chr8:144651659-144651706/C8orf73 (+3245), GSDMD (+11206); chr8:14465
9987-144660143/NAPRT1 (+448); chr8:144660548-144660589/NAPRT1 (−56); chr8:144664561-144664600/NA
PRT1 (−4068); chr8:144717844-144717871/TSTA3 (−18126), ZNF623 (−14096); chr8:144789812-144789822/M
APK15 (−8690), ZNF707 (+23195); chr8:144798641-144798676/MAPK15 (+152); chr8:144815966-144815984/F
AM83H (−61); chr8:144853523-144853582/FAM83H (−37639), SCRIB (+43996); chr8:144922907-144922959/N
RBP2 (+213); chr8:144965156-144965253/EPPK1 (−17771), PLEC (+59839); chr8:145049262-145049284/PLE
C (−24229), PARP10 (+11362); chr8:145103611-145103715/OPLAH (+11921), SPATC1 (+17081); chr8:145103
905-145103950/OPLAH (+11656), SPATC1 (+17346); chr8:145104391-145104420/OPLAH (+11178), SPATC1
(+17824); chr8:145104664-145104766/OPLAH (+10869), SPATC1 (+18133); chr8:145105890-145105948/OPL
AH (+9665), SPATC1 (+19337); chr8:145106074-145106173/OPLAH (+9460), SPATC1 (+19542); chr8:145106
348-145106415/OPLAH (+9202), SPATC1 (+19800); chr8:145106833-145107011/OPLAH (+8662), SPATC1
(+20340); chr8:145129242-145129294/EXOSC4 (−4254); chr8:145239579-145239641/HEATR7A (+36691), BOP1
(+91570); chr8:145557228-145557292/DGAT1 (−6693), SCRT1 (+2683); chr8:145557516-145557599/DGAT1
(−6991), SCRT1 (+2385); chr8:145559887-145560005/SCRT1 (−3); chr8:145582682-145582713/C8ORFK29 (−41
24), FBXL6 (−515), GPR172A (+422); chr8:145653887-145653907/VPS28 (+30); chr8:145691793-145691818/C
YHR1 (−1327), KIFC2 (+68); chr8:145691938-145692043/CYHR1 (−1512), KIFC2 (+253); chr8:145697983-1456
98378/FOXH1 (+3537), KIFC2 (+6443); chr8:145703038-145703068/FOXH1 (−1335); chr8:145725852-1457260
06/GPT (−3536); chr8:145734620-145734656/MFSD3 (+86); chr8:145735246-145735280/MFSD3 (+711); chr8:1
45747509-145747523/RECQL4 (−4306), LRRC14 (+4153); chr8:145747743-145748173/RECQL4 (−4748), LRR
C14 (+4595); chr8:145748382-145748486/LRRC24 (+3982), LRRC14 (+5071); chr8:145755318-145755365/LR
RC24 (−2926); chr8:145806625-145806709/LRRC24 (−54251), ARHGAP39 (+32221); chr8:145900764-1459007
80/ARHGAP39 (−61884), ZNF251 (+80198); chr8:145901141-145901189/ARHGAP39 (−62277), ZNF251 (+7980
5); chr8:145910352-145910371/ARHGAP39 (−71474), ZNF251 (+70608); chr8:145910827-145910849/ARHGA
P39 (−71950), ZNF251 (+70132); chr8:145911757-145911813/ARHGAP39 (−72897), ZNF251 (+69185); chr8:14
5925429-145925458/ARHGAP39 (−86556), ZNF251 (+55526); chr8:145926069-145926099/ARHGAP39 (−8719
6), ZNF251 (+54886); chr8:145938498-145938528/ARHGAP39 (−99625), ZNF251 (+42457); chr8:145938693-1
45938722/ARHGAP39 (−99820), ZNF251 (+42262); chr8:145938866-145938960/ARHGAP39 (−100025), ZNF25
1 (+42057); chr8:145955580-145955702/ARHGAP39 (−116753), ZNF251 (+25329); chr8:145980676-14598071
9/ZNF251 (+272); chr8:146017556-146017579/ZNF34 (−4843), RPL8 (+237); chr8:146126850-146126867/ZNF
250 (−13); chr8:146127827-146127853/ZNF250 (−994); chr8:146276473-146276556/ZNF252 (−48230); chr9:117
960-118139/FOXD4 (+367); chr9:707111-707165/DMRT1 (−134552), KANK1 (+202435); chr9:841406-841449/
DMRT1 (−262); chr9:842032-842207/DMRT1 (+430); chr9:842533-842610/DMRT1 (+882); chr9:842748-842779/
DMRT3 (−134200), DMRT1 (+1074); chr9:969615-969667/DMRT3 (−7323), DMRT1 (+127951); chr9:970105-97
0185/DMRT3 (−6819), DMRT1 (+128455); chr9:970912-970992/DMRT3 (−6012), DMRT1 (+129262); chr9:9713
39-971638/DMRT3 (−5475), DMRT1 (+129799); chr9:972169-972189/DMRT3 (−4785); chr9:973021-973183/DM
RT3 (−3862); chr9:1042501-1042615/DMRT2 (−8062), DMRT3 (+65594); chr9:1045772-1045840/DMRT2 (−4814);
chr9:1046043-1046097/DMRT2 (−4550); chr9:1046219-1046379/DMRT2 (−4321); chr9:2621036-2621093/VLD
LR (−728); chr9:4489679-4489712/SLC1A1 (−731); chr9:4680366-4680418/CDC37L1 (+826); chr9:4792648-479
2677/RCL1 (−171); chr9:5628513-5628586/KIAA1432 (−569); chr9:6007416-6007439/RANBP6 (+8212), MLANA
(+116519); chr9:6644368-6644539/GLDC (+1238), UHRF2 (+231303); chr9:8857347-8857406/NONE; chr9:10
612991-10613046/PTPRD (−296); chr9:13278420-13278571/MPDZ (−28125); chr9:14346066-14346184/NFIB
(−32080), ZDHHC21 (+347355); chr9:14350012-14350046/NFIB (−35984), ZDHHC21 (+343451); chr9:14693316-
14693379/ZDHHC21 (+132); chr9:14993255-14993662/FREM1 (−82466), TTC39B (+313899); chr9:15306946-1
5306998/TTC39B (+386); chr9:16154671-16154745/PSIP1 (−643705), BNC2 (+716078); chr9:17294995-17295
056/SH3GL2 (−283927), CNTLN (+159988); chr9:17906433-17906460/ADAMTSL1 (−567632), SH3GL2 (+32749
4); chr9:17907371-17907450/ADAMTSL1 (−566668), SH3GL2 (+328458); chr9:18438719-18438768/ADAMTSL
1 (−35335), SH3GL2 (+859791); chr9:19788902-19788934/SLC24A2 (−1901); chr9:19934444-19934502/SLC24
A2 (−147456), MLLT3 (+688041); chr9:21335631-21335700/KLHL9 (−237); chr9:21965425-21965569/CDKN2A
(+9635), MTAP (+162862); chr9:21968434-21968456/CDKN2A (+6687), MTAP (+165810); chr9:21969688-2196
9747/CDKN2A (+5414), MTAP (+167083); chr9:21971155-21971184/CDKN2A (+3962), MTAP (+168535); chr9:
21974077-21974181/CDKN2A (+1003), MTAP (+171494); chr9:21975117-21975190/CDKN2A (−22); chr9:2200
6153-22006227/CDKN2A (−11700), CDKN2B (+3122); chr9:23820842-23821051/ELAVL2 (+5116); chr9:238313
71-23831450/ELAVL2 (−5348); chr9:27529104-27529157/MOB3B (+719); chr9:29212171-29212210/LINGO2
(−492888); chr9:32551244-32551271/DDX58 (−24936), TOPORS (+1368); chr9:32572966-32573026/NDUFB6
(+186); chr9:32782936-32783083/TMEM215 (−487); chr9:32783221-32783259/TMEM215 (−257); chr9:32783420-
32783590/TMEM215 (+8); chr9:33024949-33025024/DNAJA1 (−222); chr9:33076435-33076517/SMU1 (+238);
chr9:33167597-33167644/B4GALT1 (−265); chr9:33290318-33290342/NFX1 (−88); chr9:34381366-34381416/KI
AA1161 (−4497); chr9:34577992-34578082/ENHO (−55000), CNTFR (+11698); chr9:34578256-34578408/ENHO
(−55295), CNTFR (+11403); chr9:34591912-34591969/CNTFR (−2206); chr9:34593053-34593243/CNTFR (−34
13); chr9:34622918-34622970/DCTN3 (−2448); chr9:34624586-34624646/DCTN3 (−4120); chr9:34629077-3462
9094/ARID3C (−1075); chr9:34629292-34629341/ARID3C (−1306); chr9:34809559-34809876/KIAA1045 (−14847
4), FAM205A (−80183); chr9:35490323-35490469/RUSC2 (+389); chr9:35649720-35649823/CD72 (−31348), SI
T1 (+1175); chr9:35665028-35665066/C9orf100 (+231); chr9:35690141-35690163/TPM2 (−99); chr9:35731936-
35731976/CREB3 (−361), TLN1 (+436); chr9:35791707-35791745/NPR2 (−680); chr9:35815153-35815218/SPA
G8 (−2927), HINT2 (−144); chr9:35846432-35846468/TMEM8B (+17221), OR13J1 (+23948); chr9:35972182-35
972320/RECK (−64659), OR2S2 (−14100); chr9:36258646-36258697/GNE (+18381), CLTA (+67819); chr9:3645
8538-36458603/MELK (−114334), RNF38 (−58275); chr9:36739598-36739730/MELK (+166759), PAX5 (+29481
2); chr9:36986097-36986116/PAX5 (+48369), MELK (+413202); chr9:36986787-36986847/PAX5 (+47659), ME
LK (+413912); chr9:37002602-37002818/PAX5 (+31766), MELK (+429805); chr9:37026352-37026433/PAX5
(+8083), MELK (+453488); chr9:37027272-37027324/PAX5 (+7178), MELK (+454393); chr9:37465034-37465217/
ZBTB5 (+281); chr9:37800567-37800633/DCAF10 (−190); chr9:38392941-38393000/ALDH1B1 (+310); chr9:38
621259-38621329/IGFBPL1 (−196850), CNTNAP3 (+667006); chr9:40332461-40332543/FAM75A3 (−367789), F
AM75A2 (+447527); chr9:42718286-42718298/FOXD4L4 (+1058), FAM75A6 (+912438); chr9:43134565-43134
611/FOXD4L4 (+417354), FAM75A6 (+496142); chr9:43134940-43134982/FOXD4L4 (+417727), FAM75A6 (+4
95769); chr9:43685188-43685276/CNTNAP3B (+347); chr9:68377662-68377694/ANKRD20A3 (+450917), FOX
D4L6 (+824526); chr9:68410945-68410969/ANKRD20A3 (+484196), FOXD4L6 (+791247); chr9:68411138-684
11152/ANKRD20A3 (+484384), FOXD4L6 (+791059); chr9:68413176-68413225/ANKRD20A3 (+486440), FOX
D4L6 (+789003); chr9:69196800-69196930/FOXD4L6 (+5339); chr9:69197259-69197435/FOXD4L6 (+4857); c
hr9:69201441-69201530/FOXD4L6 (+718); chr9:70922116-70922353/PGM5 (−49580), FOXD4L3 (+4452); chr9:
70922512-70922806/PGM5 (−49156), FOXD4L3 (+4876); chr9:70923053-70923114/PGM5 (−48731), FOXD4L3
(+5301); chr9:70972154-70972339/PGM5 (+432); chr9:71735730-71735786/TJP2 (−84320), FXN (+85279); chr
9:71789261-71789452/TJP2 (−30721), FXN (+138878); chr9:71940216-71940263/FAM189A2 (+752); chr9:7228
7591-72287646/APBA1 (−344); chr9:74061759-74061787/TRPM3 (−325259), TMEM2 (+322027); chr9:7497882
4-74978858/ZFAND5 (+1322), GDA (+214574); chr9:77113341-77113558/RORB (+1198), TRPM6 (+389186); c
hr9:77113709-77113805/RORB (+1505), TRPM6 (+388879); chr9:77642574-77642635/TRPM6 (−139969), C9or
f95 (+60528); chr9:77643022-77643040/TRPM6 (−140395), C9orf95 (+60102); chr9:79628941-79629207/PRUN
E2 (−108071), FOXB2 (−5497); chr9:79630491-79630544/FOXB2 (−4053); chr9:79635675-79635835/VPS13A
(−156606), FOXB2 (+1184); chr9:79637900-79637977/VPS13A (−154422), FOXB2 (+3368); chr9:80262552-8026
2682/GNA14 (+615); chr9:80850855-80850907/CEP78 (−110); chr9:84303166-84303267/TLE1 (+379); chr9:843
03940-84304028/TLE1 (−388); chr9:84304747-84304776/TLE1 (−1166); chr9:86152372-86152381/FRMD3 (+97
1); chr9:86322953-86322998/AK300656 (−260), UBQLN1 (+192); chr9:86535969-86536018/KIF27 (+348); chr9:
86536415-86536450/KIF27 (−91); chr9:86571709-86571751/C9orf64 (−67); chr9:87285196-87285272/NTRK2
(+1768); chr9:87285533-87285554/NTRK2 (+2078); chr9:87905281-87905324/AGTPBP1 (+451641), NTRK2 (+6
21837); chr9:88137726-88137874/AGTPBP1 (+219144), NTRK2 (+854334); chr9:88357102-88357128/AGTPB
P1 (−171); chr9:89517655-89517860/ZCCHC6 (−548356), GAS1 (+44346); chr9:90559100-90559173/CDK20
(+30558), C9orf79 (+61365); chr9:90691989-90692049/SPIN1 (−311278), CDK20 (−102324); chr9:90748552-9074
8602/SPIN1 (−254720), CDK20 (−158882); chr9:91150765-91150902/C9orf47 (−454944), SPIN1 (+147537); chr9:
91605694-91605781/S1PR3 (−624), C9orf47 (−40); chr9:91925704-91925737/CKS2 (−392); chr9:92099018-920
99113/SEMA4D (−4455); chr9:92218225-92218298/GADD45G (−1665); chr9:92255461-92255525/GADD45G
(+35566); chr9:93601604-93601739/SYK (+37660), AUH (+522534); chr9:93605351-93605452/SYK (+41390), A
UH (+518804); chr9:93925316-93925466/AUH (+198815), SYK (+361379); chr9:94123828-94123862/AUH (+36
1); chr9:94185187-94185224/NFIL3 (+938); chr9:94186190-94186208/NFIL3 (−55); chr9:94868318-94868369/R
OR2 (−155900), SPTLC1 (+9346); chr9:95820962-95821006/SUSD3 (−5); chr9:95821847-95821940/SUSD3 (+9
05); chr9:95845510-95845599/C9orf89 (−12895), SUSD3 (+24566); chr9:95947472-95947484/WNK2 (+266); ch
r9:95947587-95947609/WNK2 (+386); chr9:96030944-96031042/FAM120A (−183180), WNK2 (+83781); chr9:9
6108543-96108600/FAM120A (−105601), WNK2 (+161360); chr9:96588740-96588857/BARX1 (+128809), PHF
2 (+249890); chr9:96671413-96671440/BARX1 (+46181), PHF2 (+332518); chr9:96708116-96708199/BARX1
(+9450), PHF2 (+369249); chr9:96711447-96711534/BARX1 (+6117), PHF2 (+372582); chr9:96714034-967140
68/BARX1 (+3557), PHF2 (+375142); chr9:96714224-96714465/BARX1 (+3263), PHF2 (+375436); chr9:96715
373-96715580/BARX1 (+2131), PHF2 (+376568); chr9:96716331-96716426/BARX1 (+1229), PHF2 (+377470);
chr9:96716824-96716904/BARX1 (+744); chr9:96717428-96717449/BARX1 (+169); chr9:96717807-96717840/
BARX1 (−216); chr9:96721623-96721680/BARX1 (−4044); chr9:96722548-96722727/PTPDC1 (−124073), BARX
1 (−5030); chr9:96723160-96723170/PTPDC1 (−123546), BARX1 (−5557); chr9:96792972-96792999/BARX1 (−7
5378), PTPDC1 (−53725); chr9:97489076-97489090/FBP1 (−87160), C9orf3 (−32848); chr9:97767000-97767073/
C9orf3 (+245106), FANCC (+312954); chr9:98079639-98079695/FANCC (+324); chr9:98111561-98111894/FA
NCC (−31737), PTCH1 (+159103); chr9:98112158-98112343/FANCC (−32260), PTCH1 (+158580); chr9:983154
49-98315536/C9orf102 (−322407), PTCH1 (−44662); chr9:98332508-98332577/C9orf102 (−305357), PTCH1 (−61
712); chr9:98790013-98790083/C9orf102 (+152148), HSD17B3 (+274386); chr9:99181473-99181499/ZNF367
(−817); chr9:99482424-99482468/C9orf21 (−64847), ZNF510 (+57882); chr9:99616671-99616725/ZNF782 (−309);
chr9:99839150-99839218/CTSL2 (−37592), ZNF322 (+122668); chr9:99984058-99984107/C9orf174 (−85827),
ZNF322 (−22231); chr9:100610135-100610200/XPA (−150477), FOXE1 (−5369); chr9:100610817-100611021/F
OXE1 (−4618); chr9:100614000-100614192/FOXE1 (−1441); chr9:100614422-100614441/FOXE1 (−1105); chr9:
100616036-100616158/FOXE1 (+560); chr9:100616883-100616914/FOXE1 (+1362), C9orf156 (+67953); chr9:
100617079-100617251/FOXE1 (+1628), C9orf156 (+67687); chr9:100684498-100684527/C9orf156 (+339); chr
9:100746987-100747056/NANS (−71937), ANP32B (+1533); chr9:100954795-100954854/CORO2A (+131); chr
9:101338126-101338240/TBC1D2 (−320180), GABBR2 (+133296); chr9:101706196-101706312/COL15A1 (+11
6); chr9:101984595-101984672/ALG2 (−388), SEC61B (+64); chr9:102590760-102590859/STX17 (−78105), NR
4A3 (+6673); chr9:102668853-102668892/STX17 (−42); chr9:103235258-103235307/MURC (−105053), C9orf30-
TMEFF1 (+31095); chr9:104248650-104248697/C9orf125 (+801); chr9:104500182-104500207/GRIN3A (+667);
chr9:107689044-107689101/ABCA1 (+1454), NIPSNAP3B (+162622); chr9:108320045-108320100/FKTN (−33
8); chr9:110045476-110045505/RAD23B (−26); chr9:110249659-110249748/KLF4 (+2343), RAD23B (+204187);
chr9:110249939-110250473/KLF4 (+1841), RAD23B (+204689); chr9:110250619-110250630/KLF4 (+1422), R
AD23B (+205108); chr9:110253827-110253904/KLF4 (−1819); chr9:110947154-110947259/KLF4 (−695160), AC
TL7B (+671068); chr9:111929023-111929077/C9orf4 (+521); chr9:111929318-111929339/C9orf4 (+242); chr9:
111929528-111929585/C9orf4 (+14); chr9:112172501-112172594/EPB41L4B (−89527), PTPN3 (+88045); chr9:
112810629-112810659/TXN (+208276), PALM2-AKAP2 (+268067); chr9:113017971-113018040/TXN (+914); c
hr9:113341622-113341679/SVEP1 (+509); chr9:114245430-114245457/OR2K2 (−154731), KIAA0368 (+1581);
chr9:114423605-114423654/UGCG (−235576), DNAJC25 (+29998); chr9:115480459-115480497/C9orf80 (−91);
chr9:115512653-115512715/SNX30 (−450); chr9:116037377-116037422/PRPF4 (−514), CDC26 (+469); chr9:11
6172762-116172926/POLE3 (+185); chr9:116173071-116173088/POLE3 (−51); chr9:116350943-116351042/ZN
F618 (−287569), RGS3 (+143982); chr9:116450232-116450327/ZNF618 (−188282), RGS3 (+243269); chr9:117
266845-117267009/DFNB31 (+809); chr9:119722189-119722227/TRIM32 (+272627), ASTN2 (+455109); chr9:
120507299-120507309/TLR4 (+40844); chr9:122131439-122131496/DBC1 (+271); chr9:122132026-12213205
1/DBC1 (−300); chr9:123639542-123639588/PHF19 (+41); chr9:123690524-123690585/TRAF1 (+896); chr9:12
4062126-124062156/GSN (+62); chr9:124132907-124132982/STOM (−400); chr9:124291875-124292047/DAB2
IP (−37438), GGTA1P (−29655); chr9:124313124-124313185/GGTA1P (−50849), DAB2IP (−16244); chr9:124461
423-124461448/DAB2IP (+132037), TTLL11 (+394449); chr9:124498632-124498691/DAB2IP (+169263), TTLL
11 (+357223); chr9:124981634-124981877/NDUFA8 (−59658), LHX6 (+9335); chr9:124982176-124982244/ND
UFA8 (−60112), LHX6 (+8881); chr9:124990786-124990828/LHX6 (+284); chr9:125675559-125675612/ZBTB6
(+23); chr9:126030352-126030391/STRBP (+483); chr9:126101783-126101846/STRBP (−70960), CRB2 (−16633);
chr9:126135972-126136049/CRB2 (+17563), DENND1A (+556406); chr9:126183104-126183193/CRB2 (+64
701), DENND1A (+509268); chr9:126692085-126692094/DENND1A (+327); chr9:126762209-126762231/DEN
ND1A (−69803), LHX2 (−11669); chr9:126774278-126774459/LHX2 (+480); chr9:126774620-126775014/LHX2
(+928); chr9:126775183-126775262/NEK6 (−244973), LHX2 (+1334); chr9:126776099-126776270/NEK6 (−2440
11), LHX2 (+2296); chr9:126777816-126777973/NEK6 (−242301), LHX2 (+4006); chr9:126778239-126778358/
NEK6 (−241897), LHX2 (+4410); chr9:126779162-126779292/NEK6 (−240969), LHX2 (+5338); chr9:126780185-
126780281/NEK6 (−239963), LHX2 (+6344); chr9:127215590-127215678/GPR144 (+2211), NR5A1 (+54065); c
hr9:127258042-127258187/NR5A1 (+11584), GPR144 (+44692); chr9:127265625-127265692/NR5A1 (+4040),
GPR144 (+52236); chr9:127266588-127266628/NR5A1 (+3091), GPR144 (+53185); chr9:127266847-1272668
65/NR5A1 (+2843), GPR144 (+53433); chr9:128003043-128003087/HSPA5 (+601); chr9:128003311-12800335
0/HSPA5 (+335); chr9:128469279-128469311/MAPKAP1 (+218); chr9:128509666-128509705/PBX3 (+69); chr9:
128651564-128651649/FAM125B (−437516), PBX3 (+141990); chr9:128985404-128985465/FAM125B (−10368
8), PBX3 (+475818); chr9:129262179-129262308/LMX1B (−114478), FAM125B (+173121); chr9:129263022-12
9263128/LMX1B (−113647), FAM125B (+173952); chr9:129276933-129276961/LMX1B (−99775), FAM125B (+1
87824); chr9:129373038-129373250/LMX1B (−3578); chr9:129377363-129377452/LMX1B (+686); chr9:129377
636-129377772/LMX1B (+982); chr9:129377960-129378024/ZBTB43 (−189293), LMX1B (+1270); chr9:1293810
84-129381298/ZBTB43 (−186094), LMX1B (+4469); chr9:129381529-129381591/ZBTB43 (−185725), LMX1B
(+4838); chr9:129381937-129382041/ZBTB43 (−185296), LMX1B (+5267); chr9:129386197-129386429/ZBTB43
(−180972), LMX1B (+9591); chr9:129387749-129387847/ZBTB43 (−179487), LMX1B (+11076); chr9:129388754-
129388816/ZBTB43 (−178500), LMX1B (+12063); chr9:129412866-129412923/ZBTB43 (−154390), LMX1B (+36
173); chr9:129485772-129486023/ZBTB43 (−81387), LMX1B (+109176); chr9:130330605-130330630/FAM129B
(+737); chr9:130370668-130370712/STXBP1 (−3796); chr9:130497262-130497310/TOR2A (+342); chr9:13054
5434-130545531/SH2D3C (−4435), CDK9 (−2822); chr9:130564950-130565008/FPGS (−175); chr9:130616753-
130616856/ENG (+242); chr9:130689668-130689741/ST6GALNAC4 (−10400), PIP5KL1 (+3371); chr9:1306901
67-130690197/ST6GALNAC4 (−10877), PIP5KL1 (+2894); chr9:130768366-130768475/DPM2 (−67658), NAIF1
(+61178); chr9:130890801-130890882/PTGES2 (−368), LOC389791 (+34); chr9:131013068-131013198/GOLG
A2 (+25135), DNM1 (+47470); chr9:131013355-131013399/GOLGA2 (+24891), DNM1 (+47714); chr9:1312672
26-131267275/GLE1 (+280); chr9:131451341-131451386/SET (−145); chr9:131451914-131451950/SET (+423);
chr9:131465725-131465810/PKN3 (+966); chr9:131592046-131592075/C9orf114 (+39); chr9:131709800-1317
09841/NUP188 (−156), DOLK (+191); chr9:131863108-131863235/CRAT (+9898), DOLPP1 (+19789); chr9:131
872845-131872856/PPP2R4 (−377), CRAT (+219); chr9:131940546-131940585/METTL11A (−447869), PPP2R4
(+67338); chr9:132099434-132099658/METTL11A (−288889), PPP2R4 (+226318); chr9:132113495-13211354
8/METTL11A (−274913), PPP2R4 (+240294); chr9:132175579-132175603/METTL11A (−212844), PPP2R4 (+30
2363); chr9:132200804-132200890/METTL11A (−187588), PPP2R4 (+327619); chr9:132312568-132312620/M
ETTL11A (−75841), PPP2R4 (+439366); chr9:132372653-132372704/METTL11A (−15756), PPP2R4 (+499451);
chr9:132382363-132382432/METTL11A (−6037), PPP2R4 (+509170); chr9:132650763-132650997/USP20 (+5
3184), FNBP1 (+154593); chr9:132804662-132804693/FNBP1 (+795); chr9:132804979-132805028/FNBP1 (+4
69); chr9:133027814-133027870/AK074396 (−277827), NCS1 (+92985); chr9:133028170-133028194/AK07439
6 (−277487), NCS1 (+93325); chr9:133308747-133308769/ASS1 (−11336), AK074396 (+3089); chr9:133320834-
133320884/ASS1 (+765); chr9:133534534-133534580/PRDM12 (−5424), FUBP3 (+79597); chr9:133535561-13
3535706/PRDM12 (−4347); chr9:133535934-133535971/PRDM12 (−4028); chr9:133536120-133536149/PRDM1
2 (−3846); chr9:133539424-133539510/PRDM12 (−514); chr9:133815477-133815528/FIBCD1 (−1264); chr9:133
815671-133815730/FIBCD1 (−1462); chr9:134114159-134114279/PPAPDC3 (−50862), NUP214 (+113238); chr
9:134128101-134128239/PPAPDC3 (−36911), NUP214 (+127189); chr9:134131738-134131797/PPAPDC3 (−33
313), NUP214 (+130787); chr9:134148647-134148713/PPAPDC3 (−16401), NUP214 (+147699); chr9:1341513
72-134151449/PPAPDC3 (−13670), NUP214 (+150430); chr9:134158196-134158229/PPAPDC3 (−6868), NUP2
14 (+157232); chr9:134406368-134406406/UCK1 (+275); chr9:134420710-134420759/UCK1 (−14073), RAPGE
F1 (+164494); chr9:134430007-134430124/UCK1 (−23404), RAPGEF1 (+155163); chr9:134430259-134430482/
UCK1 (−23709), RAPGEF1 (+154858); chr9:134465863-134465963/UCK1 (−59251), RAPGEF1 (+119316); chr9:
134609056-134609145/RAPGEF1 (−23872), MED27 (+346173); chr9:134955018-134955069/MED27 (+230); c
hr9:135037288-135037333/NTNG2 (−23); chr9:135038264-135038352/NTNG2 (+974); chr9:135039740-135039
939/NTNG2 (+2506), SETX (+190532); chr9:135230423-135230447/SETX (−63); chr9:135285865-135285880/T
TF1 (−3635); chr9:135406874-135406975/TTF1 (−124687), BARHL1 (−51068); chr9:135455244-135455287/BAR
HL1 (−2727); chr9:135456024-135456079/BARHL1 (−1941); chr9:135456205-135456234/BARHL1 (−1773); chr9:
135458440-135458456/BARHL1 (+455); chr9:135462078-135462647/GTF3C4 (−83365), BARHL1 (+4370); chr9:
135466065-135466117/GTF3C4 (−79637), BARHL1 (+8098); chr9:135545627-135545644/GTF3C4 (−92), DDX
31 (+152); chr9:135545824-135545857/DDX31 (−53), GTF3C4 (+113); chr9:136024969-136025000/RALGDS
(−28424), OBP2B (+59643); chr9:136150135-136150292/ABO (+416); chr9:136214785-136214803/RPL7A (−275),
MED22 (+178); chr9:136243049-136243087/C9orf96 (−216), SURF4 (−98); chr9:136294774-136294864/C9orf7
(−30268), ADAMTS13 (+7699); chr9:136294968-136294999/C9orf7 (−30103), ADAMTS13 (+7864); chr9:13635
3075-136353125/SLC2A6 (−8824), TMEM8C (+36968); chr9:136399390-136399431/ADAMTSL2 (−564); chr9:13
6444239-136444418/FAM163B (+1039), ADAMTSL2 (+44354); chr9:136451015-136451046/DBH (−50454), FA
M163B (−5663); chr9:136474224-136474260/FAM163B (−28874), DBH (−27243); chr9:136474391-136474431/F
AM163B (−29043), DBH (−27074); chr9:136474592-136474724/FAM163B (−29290), DBH (−26827); chr9:136567
554-136567711/SARDH (+35846), DBH (+66148); chr9:136710244-136710348/SARDH (−106817), VAV2 (+147
150); chr9:136771536-136771563/SARDH (−168071), VAV2 (+85896); chr9:136857480-136857626/VAV2 (−107);
chr9:136930623-136930696/VAV2 (−73214), BRD3 (+2481); chr9:137028717-137028754/RXRA (−189580), W
DR5 (+27526); chr9:137125004-137125088/RXRA (−93270), WDR5 (+123836); chr9:137156002-137156049/RX
RA (−62290), WDR5 (+154816); chr9:137185845-137185895/RXRA (−32446), WDR5 (+184660); chr9:13721875
7-137218787/RXRA (+456); chr9:137230495-137230543/COL5A1 (−303133), RXRA (+12203); chr9:137288499-
137288599/COL5A1 (−245103), RXRA (+70233); chr9:137297212-137297335/COL5A1 (−236378), RXRA (+78
958); chr9:137347952-137348022/COL5A1 (−185665), RXRA (+129671); chr9:137378545-137378637/COL5A1
(−155061), RXRA (+160275); chr9:137533967-137534065/COL5A1 (+364); chr9:137662852-137663009/FCN2
(−109727), COL5A1 (+129279); chr9:137820780-137820912/OLFM1 (−146243), FCN1 (−11040); chr9:137936301-
137936393/FCN1 (−126541), OLFM1 (−30742); chr9:137967113-137967132/OLFM1 (+34); chr9:137967369-13
7967465/OLFM1 (+328); chr9:137967651-137967688/OLFM1 (+581); chr9:137980144-137980216/PPP1R26
(−391468), OLFM1 (+13091); chr9:137990396-137990442/PPP1R26 (−381229), OLFM1 (+23330); chr9:1381360
69-138136084/PPP1R26 (−235571), OLFM1 (+168988); chr9:138391511-138391563/MRPS2 (−946); chr9:1384
76818-138476901/PAEP (+23256), GLT6D1 (+54526); chr9:138606461-138606510/KCNT1 (+12455), CAMSA
P1 (+192519); chr9:138606774-138606820/KCNT1 (+12766), CAMSAP1 (+192208); chr9:138799677-1387997
40/CAMSAP1 (−704); chr9:138818910-138819032/CAMSAP1 (−19966), UBAC1 (+34255); chr9:138852883-138
852950/UBAC1 (+309); chr9:138987157-138987167/NACC2 (−31); chr9:138990657-138990811/NACC2 (−3603);
chr9:138999194-138999263/NACC2 (−12098), LHX3 (+95775); chr9:139002564-139002798/NACC2 (−15550),
LHX3 (+92323); chr9:139010698-139010718/NACC2 (−23577), LHX3 (+84296); chr9:139014599-139014645/N
ACC2 (−27491), LHX3 (+80382); chr9:139085351-139085372/NACC2 (−98231), LHX3 (+9642); chr9:139085828-
139085985/NACC2 (−98776), LHX3 (+9097); chr9:139090552-139090586/NACC2 (−103438), LHX3 (+4435); ch
r9:139090742-139091071/NACC2 (−103776), LHX3 (+4097); chr9:139091525-139091706/NACC2 (−104485), L
HX3 (+3388); chr9:139093719-139093734/NACC2 (−106596), LHX3 (+1277); chr9:139093923-139094021/NAC
C2 (−106841), LHX3 (+1032); chr9:139095131-139095165/LHX3 (−144), LHX3 (−144); chr9:139137703-1391377
31/QSOX2 (−30); chr9:139221115-139221229/GPSM1 (−760); chr9:139258023-139258110/DNLZ (+174); chr9:1
39305433-139305474/SDCCAG3 (−400), PMPCA (+338); chr9:139424551-139424589/SEC16A (−47063), NOT
CH1 (+15668); chr9:139425228-139425355/SEC16A (−47785), NOTCH1 (+14946); chr9:139427226-139427308/
SEC16A (−49760), NOTCH1 (+12971); chr9:139428489-139428569/SEC16A (−51022), NOTCH1 (+11709); chr
9:139440038-139440052/NOTCH1 (+193); chr9:139472838-139472898/EGFL7 (−87356), NOTCH1 (−32630); c
hr9:139481576-139481634/EGFL7 (−78619), NOTCH1 (−41367); chr9:139482422-139482443/EGFL7 (−77791),
NOTCH1 (−42195); chr9:139482731-139482817/EGFL7 (−77450), NOTCH1 (−42536); chr9:139491049-1394911
07/EGFL7 (−69146), NOTCH1 (−50840); chr9:139494626-139494723/EGFL7 (−65549), NOTCH1 (−54437); chr9:
139498556-139498630/EGFL7 (−61631), NOTCH1 (−58355); chr9:139514305-139514341/NOTCH1 (−74085), E
GFL7 (−45901); chr9:139522722-139522765/NOTCH1 (−82506), EGFL7 (−37480); chr9:139522914-139522979/
NOTCH1 (−82709), EGFL7 (−37277); chr9:139539834-139539967/NOTCH1 (−99663), EGFL7 (−20323); chr9:13
9540411-139540462/NOTCH1 (−100199), EGFL7 (−19787); chr9:139540800-139540894/NOTCH1 (−100609), E
GFL7 (−19377); chr9:139541503-139541552/NOTCH1 (−101290), EGFL7 (−18696); chr9:139548352-139548395/
NOTCH1 (−108136), EGFL7 (−11850); chr9:139552909-139552982/NOTCH1 (−112708), EGFL7 (−7278); chr9:1
39553136-139553722/NOTCH1 (−113191), EGFL7 (−6795); chr9:139553876-139553911/NOTCH1 (−113656), E
GFL7 (−6330); chr9:139555090-139555133/NOTCH1 (−114874), EGFL7 (−5112); chr9:139559649-139559749/E
GFL7 (−525); chr9:139560192-139560220/EGFL7 (−18); chr9:139560801-139560828/EGFL7 (+591); chr9:1395
81917-139581927/AGPAT2 (−11); chr9:139685772-139685800/TMEM141 (+9); chr9:139694764-139694827/C9
orf86 (−7396), TMEM141 (+9019); chr9:139716076-139716246/PHPT1 (−27095), C9orf86 (+13969); chr9:13973
8482-139738522/PHPT1 (−4754); chr9:139738991-139739024/PHPT1 (−4248); chr9:139739133-139739447/PH
PT1 (−3966); chr9:139740229-139740338/PHPT1 (−2972); chr9:139740650-139740674/PHPT1 (−2594); chr9:13
9741016-139741046/PHPT1 (−2225); chr9:139741377-139741690/PHPT1 (−1722); chr9:139743886-139743923/
MAMDC4 (−2914), PHPT1 (+649); chr9:139879796-139879955/LCNL1 (+2431), CLIC3 (+11148); chr9:1398803
38-139880471/LCNL1 (+2960), CLIC3 (+10619); chr9:139892470-139892512/CLIC3 (−1467); chr9:140023870-1
40023901/DPP7 (−14691), GRIN1 (−9723); chr9:140024771-140024786/DPP7 (−15584), GRIN1 (−8830); chr9:14
0024919-140024956/DPP7 (−15743), GRIN1 (−8671); chr9:140033491-140033618/GRIN1 (−54); chr9:14005109
7-140051219/LRRC26 (+13333), GRIN1 (+17549); chr9:140057963-140058016/LRRC26 (+6501), GRIN1 (+243
81); chr9:140095037-140095052/TPRN (+118); chr9:140100042-140100069/TPRN (−4893), NDOR1 (−63), TME
M203 (+34); chr9:140117157-140117171/RNF224 (−4854), RNF208 (−1389); chr9:140117326-140117401/RNF2
24 (−4654), RNF208 (−1589); chr9:140172066-140172152/C9orf167 (−171); chr9:140172574-140172697/C9orf1
67 (+356); chr9:140172806-140172875/C9orf167 (+561); chr9:140173051-140173096/C9orf167 (+794); chr9:14
0189360-140189390/NRARP (+7328), C9orf167 (+17095); chr9:140210909-140210943/NRARP (−14223), EXD
3 (+106788); chr9:140329181-140329283/ENTPD8 (+6669), NOXA1 (+11385); chr9:140356218-140356341/NE
LF (−2494); chr9:140356817-140356849/NELF (−3047); chr9:140513092-140513120/EHMT1 (−338); chr9:14077
1908-140771965/CACNA1B (−304); chr9:140772594-140772756/CACNA1B (+434); chrX:3263826-3263918/MX
RA5 (+812); chrX:3265013-3265150/MXRA5 (−398); chrX:6145720-6145835/NLGN4X (+928); chrX:7895049-78
95084/PNPLA4 (+713); chrX:8699058-8699110/KAL1 (+1143), VCX3B (+266213); chrX:8699762-8699940/KAL
1 (+376); chrX:9733189-9733221/GPR143 (+800); chrX:9733437-9733845/GPR143 (+364); chrX:9753825-975
3862/SHROOM2 (−652); chrX:9983733-9983762/CLCN4 (−141237), SHROOM2 (+229252); chrX:10126796-101
26824/CLCN4 (+1825), MID1 (+724999); chrX:11445723-11445829/AMELX (+134243), ARHGAP6 (+238045);
chrX:11682697-11682722/ARHGAP6 (+1111), AMELX (+371177); chrX:11683545-11683583/ARHGAP6 (+257);
chrX:11683790-11683837/ARHGAP6 (+7); chrX:11776799-11776820/MSL3 (+532); chrX:12156658-12156679/
FRMPD4 (+84); chrX:12156794-12156857/FRMPD4 (+241); chrX:14891612-14891625/FANCB (−435), MOSPD
2 (+92); chrX:16730140-16730152/CTPS2 (+913); chrX:16889167-16889207/RBBP7 (−1209); chrX:17393425-1
7393450/NHS (−105); chrX:17395538-17395567/SCML1 (−360039), NHS (+2010); chrX:17673759-17673820/S
CML1 (−81802), NHS (+280247); chrX:17674307-17674403/SCML1 (−81237), NHS (+280812); chrX:17756494-
17756567/SCML1 (+939); chrX:18372695-18372723/SCML2 (+135); chrX:18373396-18373543/SCML2 (−626);
chrX:18904855-18904959/PHKA2 (+97573), PPEF1 (+195862); chrX:19140060-19140082/GPR64 (+684); chrX:
20284812-20284827/RPS6KA3 (−70); chrX:21676579-21676606/SMPX (+99685), CNKSR2 (+284057); chrX:21
959621-21959676/SMS (+934); chrX:23353086-23353124/PTCHD1 (+120); chrX:23685674-23685694/PRDX4
(+39); chrX:25021580-25021610/ARX (+12470), POLA1 (+309531); chrX:25022843-25022892/ARX (+11197), P
OLA1 (+310804); chrX:25025271-25025306/ARX (+8776), POLA1 (+313225); chrX:25025518-25025586/ARX
(+8513), POLA1 (+313488); chrX:25030116-25030212/ARX (+3901), POLA1 (+318100); chrX:25033674-250338
20/ARX (+318); chrX:25034013-25034132/ARX (−8); chrX:25041080-25041121/ARX (−7036); chrX:30327000-30
327058/NR0B1 (+466); chrX:30327629-30327658/NR0B1 (−149); chrX:30671055-30671152/GK (−372); chrX:30
907511-30907521/TAB3 (−5); chrX:34675056-34675111/TMEM47 (+321); chrX:36975654-36975696/PRRG1
(−232853), CXorf59 (+910622); chrX:36976018-36976074/PRRG1 (−232482), CXorf59 (+910993); chrX:37431393-
37431414/LANCL3 (+582); chrX:37706679-37706706/DYNLT3 (+196); chrX:38079898-38080125/SRPX (+165);
chrX:38080411-38080468/SRPX (−263); chrX:38421178-38421231/TSPAN7 (+474); chrX:38663094-3866312
3/MID1IP1 (+2424); chrX:38663725-38663772/MID1IP1 (+3064); chrX:38663915-38663969/MID1IP1 (+3257); c
hrX:39548471-39548640/BCOR (+408163), MID1IP1 (+887871); chrX:39680564-39680630/BCOR (+276122); c
hrX:39864973-39865285/BCOR (+91590); chrX:39868924-39869045/BCOR (+87734); chrX:39871760-3987177
8/BCOR (+84950); chrX:39943293-39943368/BCOR (+13388); chrX:39949467-39949535/BCOR (+7218); chrX:
39949799-39949948/BCOR (+6845); chrX:39953560-39953710/BCOR (+3084); chrX:39956856-39957026/BC
OR (−222); chrX:39957904-39958064/BCOR (−1265); chrX:39959507-39959550/BCOR (−2810); chrX:39967941-
39967970/ATP6AP2 (−472260), BCOR (−11237); chrX:40005220-40005243/ATP6AP2 (−434984), BCOR (−4851
3); chrX:40027370-40027644/ATP6AP2 (−412709), BCOR (−70788); chrX:40029077-40029114/ATP6AP2 (−411
120), BCOR (−72377); chrX:40031061-40031175/ATP6AP2 (−409098), BCOR (−74399); chrX:40033419-400335
32/ATP6AP2 (−406740), BCOR (−76757); chrX:40035739-40035852/ATP6AP2 (−404420), BCOR (−79077); chrX:
41782385-41782420/CASK (−116); chrX:41782552-41782573/CASK (−276); chrX:41782895-41782917/CASK
(−619); chrX:43514557-43514583/MAOA (−839); chrX:45711218-45711231/CXorf36 (−651079), ZNF673 (−595399);
chrX:46618307-46618326/SLC9A7 (+155); chrX:46618686-46618721/SLC9A7 (−232); chrX:46697006-46697
035/RP2 (+674); chrX:47092632-47092722/USP11 (+363); chrX:47230642-47230689/ZNF157 (+667); chrX:473
42460-47342497/ZNF41 (−134); chrX:47372725-47372743/ARAF (−47782), ZNF41 (−30389); chrX:47433504-47
433528/TIMP1 (−8174), ARAF (+13000); chrX:47433807-47433827/TIMP1 (−7873), ARAF (+13301); chrX:47433
972-47434030/TIMP1 (−7689), ARAF (+13485); chrX:48367202-48367222/PORCN (−960); chrX:48367450-4836
7473/PORCN (−710); chrX:48690515-48690671/ERAS (+3310), PCSK1N (+3367); chrX:48748805-48748923/P
CSK1N (−54904), PQBP1 (−6331); chrX:48749176-48749266/PCSK1N (−55261), PQBP1 (−5974); chrX:4881460
7-48814634/OTUD5 (+272); chrX:48911157-48911189/TFE3 (−10183), WDR45 (+26391); chrX:48931468-4893
1483/TFE3 (−30486), WDR45 (+6088); chrX:48979986-48979998/GPKOW (+87); chrX:49022631-49022667/GP
KOW (−42570), PLP2 (−5535); chrX:50212431-50212475/DGKK (+1268), CCNB3 (+184911); chrX:50557041-50
557074/SHROOM4 (−14); chrX:51138757-51138798/NUDT10 (+63695), NUDT11 (+100681); chrX:51139418-5
1139480/NUDT10 (+64366), NUDT11 (+100010); chrX:51239177-51239210/NUDT11 (+265); chrX:51453590-5
1453627/NUDT11 (−214150), GSPT2 (−32872); chrX:51454008-51454028/NUDT11 (−214559), GSPT2 (−32463);
chrX:51454190-51454236/NUDT11 (−214754), GSPT2 (−32268); chrX:51486665-51486869/GSPT2 (+286); chr
X:53461354-53461371/HSD17B10 (−40); chrX:53710771-53710827/HSD17B10 (−249476), HUWE1 (+2874); chr
X:54555996-54556126/GNL3L (−583); chrX:55187440-55187469/ALAS2 (−129958), MTRNR2L10 (+21489); chr
X:55516223-55516315/USP51 (−638); chrX:64626522-64626597/ZC4H2 (−430147), ZC3H12B (−82055); chrX:6
4627144-64627282/ZC4H2 (−430800), ZC3H12B (−81402); chrX:64914349-64914480/MSN (+26904), VSIG4
(+345552); chrX:65858672-65858788/AR (−905144), EDA2R (−22858); chrX:66766143-66766178/AR (+2287), OP
HN1 (+887138); chrX:67718683-67718715/YIPF6 (+75); chrX:67906221-67906253/EFNB1 (−142603), STARD8
(+38726); chrX:68348499-68348639/PJA1 (+36796), EFNB1 (+299729); chrX:68724743-68724763/FAM155B
(−325); chrX:68758315-68758592/EDA (−77457), FAM155B (+33376); chrX:69353384-69353400/IGBP1 (+74); ch
rX:69654438-69654477/DLG3 (−10247), GDPD2 (+11577); chrX:69665133-69665158/DLG3 (+441); chrX:69675
020-69675045/DLG3 (+10328), TEX11 (+453534); chrX:70473966-70473999/ZMYM3 (+55); chrX:71131379-71
131398/CXCR3 (−293022), PIN4 (−270137); chrX:71351658-71351726/CXCR3 (−513325), PIN4 (−49834); chrX:7
1524855-71524952/CITED1 (+860); chrX:71996959-71996981/DMRTC1 (−67906), PHKA1 (−62941); chrX:7258
1694-72581822/NAP1L2 (−147048), CDX4 (−85332); chrX:73641988-73642120/SLC16A2 (+969); chrX:7364263
2-73642691/SLC16A2 (+1577), RLIM (+191799); chrX:73756115-73756192/RLIM (+78307), SLC16A2 (+11506
9); chrX:74742806-74742851/ZDHHC15 (+508); chrX:82763684-82763735/POU3F4 (+441); chrX:82765601-82
765733/CYLC1 (−350503), POU3F4 (+2398); chrX:90689625-90689701/PABPC5 (+66); chrX:90689856-906900
63/PABPC5 (+363); chrX:92928395-92928507/NAP1L3 (+231); chrX:95940292-95940353/DIAPH2 (+661); chrX:
99663489-99663511/PCDH19 (+1771); chrX:99663743-99663751/PCDH19 (+1524); chrX:99663910-99663923/
PCDH19 (+1354); chrX:99664067-99664158/PCDH19 (+1158); chrX:99664897-99664907/PCDH19 (+369); chr
X:99665390-99665418/PCDH19 (−133); chrX:99665939-99665964/PCDH19 (−681); chrX:100446451-10044648
7/DRP2 (−28464), CENPI (+91671); chrX:101397327-101397372/TCEAL6 (+38); chrX:101906121-101906180/G
PRASP1 (−143); chrX:101906926-101906974/GPRASP1 (+656); chrX:102470018-102470054/BEX4 (+16); chrX:
102565927-102565962/BEX2 (−62); chrX:102862751-102862801/TCEAL3 (−58); chrX:103216979-103217122/T
MSB15B (+457); chrX:103217346-103217372/TMSB15B (+765); chrX:103217537-103217589/TMSB15B (+969);
chrX:103401637-103401656/MCART6 (+61); chrX:105066260-105066291/NRK (−260); chrX:105066413-1050
66435/NRK (−112); chrX:105066911-105066939/NRK (+389); chrX:106242935-106243004/MORC4 (+504); chr
X:106693124-106693243/NUP62CL (−243514), KIAA1817 (−150451); chrX:106750341-106750412/NUP62CL
(−300707), KIAA1817 (−93258); chrX:106960078-106960138/TSC22D3 (+58909), PRPS1 (+88454); chrX:107018
421-107018462/TSC22D3 (+575); chrX:107978667-107978899/IRS4 (+824); chrX:108868110-108868220/KCN
E1L (+228); chrX:108868406-108868440/KCNE1L (−30); chrX:109560954-109561100/AMMECR1 (+353); chrX:
109561464-109561484/AMMECR1 (−94); chrX:110038953-110039006/CHRDL1 (+96); chrX:110039255-110039
268/CHRDL1 (−186); chrX:110342372-110342425/CHRDL1 (−303323), PAK3 (−23906); chrX:110924420-110924
466/ALG13 (+97); chrX:113818984-113819094/HTR2C (+488); chrX:114468409-114468427/LUZP4 (−55874), R
BMXL3 (+44455); chrX:117250913-117250942/WDR44 (−229108), KLHL13 (−131552); chrX:117480176-117480
245/WDR44 (+175); chrX:118107618-118107646/LONRF3 (−1081); chrX:118369944-118369983/PGRMC1 (−24
7); chrX:118370379-118370460/PGRMC1 (+209); chrX:119444866-119445000/FAM70A (+458); chrX:1223176
50-122317699/GRIA3 (−421); chrX:122318423-122318507/GRIA3 (+369); chrX:123094177-123094335/STAG2
(−219); chrX:124337875-124337916/ODZ1 (−240230); chrX:124338094-124338138/ODZ1 (−240450); chrX:1243
39910-124339938/ODZ1 (−242258); chrX:125686650-125686665/DCAF12L1 (+184); chrX:128788806-1287888
19/APLN (+120); chrX:128977540-128977563/ZDHHC9 (+358); chrX:129087399-129087439/BCORL1 (−51745),
UTP14A (+47322); chrX:129114786-129114835/BCORL1 (−24353), UTP14A (+74714); chrX:129300015-1293
00060/AIFM1 (−177); chrX:129402413-129402467/ZNF280C (+482); chrX:129473604-129473636/SLC25A14
(−427); chrX:130192341-130192438/ARHGAP36 (+174); chrX:130202015-130202098/ARHGAP36 (+9841), IGSF
1 (+221346); chrX:130216173-130216204/ARHGAP36 (+23973), IGSF1 (+207214); chrX:131623698-13162371
8/MBNL3 (−49366), HS6ST2 (+471715); chrX:132091719-132091736/MBNL3 (−517386), HS6ST2 (+3695); chrX:
132547835-132547884/TFDP3 (−195484), GPC4 (+1345); chrX:132548635-132548690/GPC4 (+542); chrX:133
119761-133119817/GPC3 (−116); chrX:133306212-133306237/PHF6 (−201117), GPC3 (−186552); chrX:133306
583-133306620/PHF6 (−200740), GPC3 (−186929); chrX:133306856-133306878/PHF6 (−200475), GPC3 (−1871
94); chrX:133684957-133685022/HPRT1 (+90815), PLAC1 (+107523); chrX:135229044-135229083/FHL1 (−495);
chrX:135332989-135333101/MAP7D3 (+693); chrX:136115948-136116139/GPR101 (−2211); chrX:13611632
7-136116386/GPR101 (−2524); chrX:136509390-136509450/GPR101 (−395587), ZIC3 (−138926); chrX:1366278
97-136628107/GPR101 (−514169), ZIC3 (−20344); chrX:136631467-136631558/GPR101 (−517680), ZIC3 (−168
33); chrX:136632079-136632148/GPR101 (−518281), ZIC3 (−16232); chrX:136632957-136632994/GPR101 (−51
9143), ZIC3 (−15370); chrX:136645916-136646040/ZIC3 (−2368); chrX:136648090-136648166/ZIC3 (−218); chr
X:136648820-136648867/ZIC3 (+498); chrX:136649338-136649414/ZIC3 (+1030); chrX:136649557-136649579/
ZIC3 (+1222); chrX:136650718-136650779/ZIC3 (+2403); chrX:136651276-136651332/ZIC3 (+2958); chrX:136
656246-136656522/ZIC3 (+8038); chrX:136657262-136657339/ZIC3 (+8955); chrX:137793294-137793405/FG
F13 (+493835); chrX:138285260-138285344/FGF13 (+1883); chrX:138287200-138287355/FGF13 (−93); chrX:1
38774357-138774473/MCF2 (+15966), F9 (+161520); chrX:139014609-139014762/ATP11C (−100239), CXorf6
6 (+32991); chrX:139015385-139015429/ATP11C (−100960), CXorf66 (+32270); chrX:139521645-139521696/C
Xorf66 (−473994), SOX3 (+65554); chrX:139586417-139586439/SOX3 (+797); chrX:139587051-139587162/SO
X3 (+118); chrX:139846939-139846982/SOX3 (−259736), SPANXB2 (−237795); chrX:140270914-140270964/L
DOC1 (+371); chrX:141102956-141103026/MAGEC1 (+111349), MAGEC2 (+190085); chrX:142721540-14272
1812/SLITRK4 (+2250); chrX:142722143-142722190/SLITRK4 (+1759); chrX:142722403-142722426/SLITRK4
(+1511); chrX:144899618-144899683/SLITRK2 (−3215); chrX:144899907-144899957/SLITRK2 (−2934); chrX:14
4903097-144903114/SLITRK2 (+240); chrX:147581935-147582070/AFF2 (−136); chrX:147583219-147583272/
AFF2 (+1107); chrX:149736752-149736963/MTM1 (−189); chrX:149737608-149737645/MTM1 (+580); chrX:149
861809-149861833/MTMR1 (−48); chrX:149878992-149879083/MTMR1 (+17169), CD99L2 (+188251); chrX:15
0067686-150067724/CD99L2 (−416); chrX:150345878-150345898/GPR50 (+832); chrX:150565506-150565572/
VMA21 (−118); chrX:150863505-150863564/PRRG3 (−195); chrX:150864007-150864055/PRRG3 (+301); chrX:1
51142689-151142721/GABRE (+446); chrX:151143347-151143366/GABRE (−206); chrX:151806299-15180635
1/GABRQ (−312); chrX:151806618-151806637/GABRQ (−9); chrX:151999273-151999305/NSDHL (−222), CETN
2 (+12); chrX:151999444-151999482/CETN2 (−162), NSDHL (−48); chrX:152224659-152224688/PNMA3 (−92); c
hrX:152586915-152586939/MAGEA1 (−100811), ZNF275 (−12686); chrX:152677013-152677047/ZFP92 (−6751),
ZNF275 (+77417); chrX:152677234-152677257/ZFP92 (−6535), ZNF275 (+77633); chrX:152735875-15273594
5/HAUS7 (+693); chrX:152740380-152740448/HAUS7 (−3811); chrX:152783261-152783291/ATP2B3 (−18304),
BGN (+22929); chrX:152783454-152783578/ATP2B3 (−18064), BGN (+23169); chrX:152783791-152783820/AT
P2B3 (−17774), BGN (+23459); chrX:152864406-152864438/FAM58A (+210); chrX:152908187-152908196/DU
SP9 (+295); chrX:152928030-152928053/PNCK (+11774), DUSP9 (+20145); chrX:152939271-152939323/PNC
K (+519); chrX:152990140-152990172/BCAP31 (−607), ABCD1 (−167); chrX:153059574-153059616/SSR4 (−35),
IDH3G (+372); chrX:153059829-153059996/IDH3G (+54), SSR4 (+283); chrX:153060545-153060619/IDH3G
(−615), SSR4 (+952); chrX:153084393-153084409/IDH3G (−24434), PDZD4 (+11602); chrX:153094523-1530945
63/IDH3G (−34576), PDZD4 (+1460); chrX:153094952-153095012/IDH3G (−35015), PDZD4 (+1021); chrX:1530
96352-153096414/PDZD4 (−380); chrX:153096582-153096660/PDZD4 (−618); chrX:153144833-153144922/L1C
AM (−3479); chrX:153151733-153151765/AVPR2 (−18679), L1CAM (−10350); chrX:153152113-153152173/AVP
R2 (−18285), L1CAM (−10744); chrX:153171727-153171793/AVPR2 (+1332), ARHGAP4 (+19954); chrX:153207
031-153207052/NAA10 (−6574), RENBP (+3190); chrX:153236213-153236278/TMEM187 (−1745), HCFC1 (+57
3); chrX:153237598-153237630/HCFC1 (−795), TMEM187 (−377); chrX:153238385-153238445/HCFC1 (−1596),
TMEM187 (+424); chrX:153285077-153285127/IRAK1 (+240); chrX:153362964-153362976/MECP2 (+218); chr
X:153598874-153598901/FLNA (+4118), TKTL1 (+74861); chrX:153600042-153600152/FLNA (+2909), TKTL1
(+76070); chrX:153607708-153607763/FLNA (−4730), EMD (+139); chrX:153608083-153608128/EMD (+509); c
hrX:153665764-153665781/GDI1 (+514); chrX:153718738-153718750/UBL4A (−3812), SLC10A3 (+258); chrX:1
54033868-154033915/MPP1 (−90); chrY:9116739-9116821/TSPY4 (−58339); chrY:14532761-14532794/USP9Y
(−280382); chrY:14533210-14533221/USP9Y (−279944); chrY:21239718-21239798/CD24 (−85053), KDM5D (+6
67067); chrY:27242332-27242362/CDY1 (−525917), BPY2 (−44096)

1. A method for detecting the presence of a target nucleic acid in a mixture of nucleic acids comprising:
performing methylation haplotype analysis on a sample comprising a plurality of nucleic acids; and
determining whether said sample includes a methylation haplotype indicative of the presence of said target nucleic acid.

2. The method of claim 1, wherein said methylation haplotype analysis comprises determining the combinatorial methylation status of a plurality of methylation sites in a nucleic acid molecule.

3. The method of claim 2, wherein said plurality of methylation sites comprises at least 2, at least 3, at least 4, at least 5, at least 10, at least 20, at least 40, at least 50, at least 100, at least 200, at least 300, at least 400, at least 500 or more than 500 methylation sites indicative of the presence of the target nucleic acid in the sample.

4. (canceled)

5. (canceled)

6. (canceled)

7. (canceled)

8. (canceled)

9. The method of claim 2, wherein said plurality of methylation sites are located within a region on a single nucleic acid molecule.

10. The method of claim 9, wherein said region is about 5 bp to about 500 bp.

11. (canceled)

12. (canceled)

13. (canceled)

14. (canceled)

15. (canceled)

16. (canceled)

17. (canceled)

18. (canceled)

19. The method of claim 1, comprising determining whether said sample includes at least 2 methylation haplotypes indicative of the presence of said target nucleic acid.

20. (canceled)

21. (canceled)

22. (canceled)

23. (canceled)

24. The method of claim 1, comprising enriching the plurality of nucleic acids using selector probes.

25. The method of claim 1, comprising enriching the plurality of nucleic acids using MeDiP.

26. The method of claim 1, wherein the sample comprises genomic DNA.

27. The method of claim 26, wherein said genomic DNA is bisulfite converted.

28. (canceled)

29. The method of claim 1, wherein determining the methylation status of a plurality of methylation sites comprises contacting said plurality of nucleic acids with a plurality of probes to form hybridization complexes.

30. The method of claim 29, comprising modifying said plurality of probes of said hybridization complexes to form a plurality of modified probes.

31. The method of claim 30, wherein modifying said plurality of probes comprises extending and/or ligating said plurality of probes.

32. The method of claim 29, wherein each of said plurality of probes comprises a target-specific domain.

33. The method of claim 30, comprising amplifying said plurality of modified probes.

34. The method of claim 33, wherein each of said plurality of modified probes comprises a unique molecule identifier (UMI).

35. The method of claim 33, wherein each of said plurality of modified probes comprises a primer complementary domain.

36. The method of claim 33, wherein each of said plurality of modified probes comprises two primer complementary domains.

37. The method of claim 29, wherein said plurality of probes are padlock probes.

38. The method of claim 37, wherein said padlock probes comprise two target-specific domains and a linker sequence.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The method of claim 1, wherein said methylation haplotype analysis comprises determining the methylation status of a plurality of methylation sites which are in linkage disequilibrium with one another.

51. A method for detecting a tumor in a subject, comprising:
detecting the presence of one or more target nucleic acids in a sample comprising a plurality of nucleic acids from said subject using the method of claim 1,
wherein the presence of said one or more target nucleic acids is indicative of the presence of a tumor in said subject.

52. The method of claim 51, wherein said sample is a plasma sample isolated from peripheral blood of said subject.

53. The method of claim 51, comprising detecting the presence of at least 2 target nucleic acids in a sample comprising a plurality of nucleic acids.

54. (canceled)

55. (canceled)

56. (canceled)

57. The method of claim 51, wherein each of said one or more target nucleic acids comprises one or more methylation haplotypes that are tumor cell-indicative.

58. (canceled)

59. A method for prenatal detection of fetal chromosomal abnormality, comprising:
detecting the presence of two or more target nucleic acids in a sample comprising a plurality of nucleic acids using the method of claim 1;
calculating the amount of said two or more target nucleic acids; and
determining the presence or absence of fetal chromosomal abnormality by comparing the amount of said two or more target nucleic acids.

60. The method of claim 59, wherein said sample is a plasma sample isolated from peripheral blood of a pregnant woman.

61. (canceled)

62. (canceled)

63. The method of claim 59, comprising detecting the presence of at least 100 target nucleic acids in a sample comprising a plurality of nucleic acids.

64. (canceled)

65. (canceled)

66. The method of claim 59, wherein each of said two or more target nucleic acids comprises one or more methylation haplotypes that are fetal cell-indicative.

67. The method of claim 59, wherein said fetal chromosomal abnormality is trisomy 21 (Down syndrome), trisomy 18 (Edwards syndrome), trisomy 22 (cat eye syndrome), trisomy 13 (Patau syndrome), XO, XXX, XYY, XXY, or a combination thereof.

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)
